(19) United States
(12) Patent Application Publication
(10) Pub. No.: US 2026/0285859 A1
(43) Pub. Date: Sep. 24, 2026

(54) BI-CYCLIC PYRIMIDINE COMPOUNDS, METHODS AND USES THEREOF

(71) Applicant: Zeno Management, Inc., San Diego, CA (US)

(72) Inventors: Sophie M. Perrin, San Diego, CA (US); Brant Clayton Boren, San Diego, CA (US); Christian Cho-Hua Lee, San Diego, CA (US); Martin W. Rowbottom, San Diego, CA (US); Mohammad Al-Amin, San Diego, CA (US); Quyen-Quyen Thuy Do, San Diego, CA (US); Jin Kwang Kim, San Diego, CA (US); Suvi Tuula Marjukka Orr, San Diego, CA (US); Peter Qinhua Huang, San Diego, CA (US)

(21) Appl. No.: 19/669,470

(22) Filed: May 6, 2026

Related U.S. Application Data
(63) Continuation of application No. PCT/US2024/054713, filed on Nov. 6, 2024.
(60) Provisional application No. 63/596,844, filed on Nov. 7, 2023.
(60) Provisional application No. 63/564,861, filed on Mar. 13, 2024.
(60) Provisional application No. 63/665,814, filed on Jun. 28, 2024.

(51) Int. Cl. C07D 471/04; A61K 31/519; A61K 31/5377; C07D 491/107

(52) U.S. Cl. CPC C07D 471/04; A61K 31/519; A61K 31/5377; C07D 491/107

(57) ABSTRACT

Compounds of general Formula (I) (including pharmaceutically acceptable salts thereof) are provided herein. Such compounds, as well as pharmaceutically acceptable salts and compositions thereof, are useful for treating diseases or conditions, including conditions characterized by excessive cellular proliferation, such as cancer.

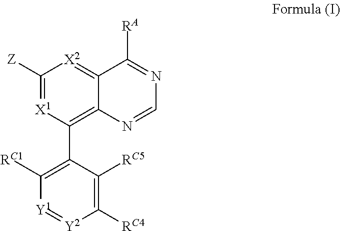

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application Nos. 63/596,844, filed Nov. 7, 2023, 63/564,861, filed Mar. 13, 2024 and 63/665,814, filed Jun. 28, 2024, each of which is incorporated by reference in their entireties. The present application is a continuation of PCT Application No. PCT/US2024/054713, filed Nov. 6, 2024, which claims priority to U.S. Provisional Application Nos. 63/596,844, filed Nov. 7, 2023, 63/564,861, filed Mar. 13, 2024, and 63/665,814, filed Jun. 28, 2024, each of which are incorporated by reference in their entireties including any drawings.

### BACKGROUND

### Field

The present application relates generally to therapeutic compounds for use in oncology, more specifically compounds that target DNA damage response mechanisms for the treatment of a broad range of cancers.

### Description

DNA is constantly damaged from the environment. Light, chemicals, stress, and cellular replication lead to single- or double-stranded breakage along DNA's backbone. Typically, organisms defend against DNA damage by repair proteins that either re-connect or re-synthesize damaged DNA. The correct functioning of these proteins are essential for life. The incorrect replacement of nucleotides into DNA can cause mutations (and other genetic alterations including but not limited to insertions, deletions, and frameshifts), genetic disease, and/or loss of protein function. The altogether loss of DNA repair can cause cell death, tumor progression, and/or cancer.

Cell cycle checkpoints are important for proper DNA repair, ensuring that cells do not progress with cellular replication until their genomic integrity is restored. CDC2 (also referred to as CDK1) promotes multiple biological processes that are critical for cell survival, including G2/M transition, checkpoint activation, DNA repair, and DNA replication. The membrane-associated tyrosine- and threonine-specific CDC2-inhibitory kinase is a regulator of CDK1 phosphorylation and is a compelling therapeutic target for the treatment of certain types of DNA damage response cancers due to its established synthetic lethal relationship with CCNE1 amplification. To date, no selective inhibitors have been reported for this kinase that would allow for investigation of the pharmacological role of the kinase.

### SUMMARY

Various embodiments provide a compound represented by the following

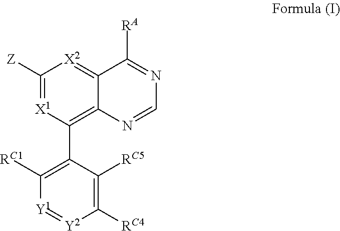

or a pharmaceutically acceptable salt thereof, wherein [symbol] and the variables X¹, X², Y¹, Y², Z, R^{A}, R^{B1}, R^{B2}, R^{C1} through R^{C5}, R¹ through R⁴⁰, p, q, and r are as defined herein.

Another embodiment provides a compound, or a pharmaceutically acceptable salt thereof, wherein the compound is represented by Formula (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or the compound has a structure selected from those of Compound Nos. 1-A to 76-C as described herein (including pharmaceutically acceptable salts thereof).

Another embodiment provides a pharmaceutical composition comprising an effective amount of a compound represented by Formula (Ia), (Ib), (Ic), (II), (II-1), (IIIa), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C as described herein, or a pharmaceutically acceptable salt of any of the foregoing, and a pharmaceutically acceptable carrier, diluent, excipient or combination thereof.

Another embodiment provides a method of ameliorating or treating a cancer in a subject comprising administering to the subject an effective amount of a compound represented by Formula (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C as described herein, or a pharmaceutically acceptable salt of any of the foregoing, or a pharmaceutical composition thereof.

Another embodiment provides a method of inhibiting replication of a malignant growth or a tumor in a subject comprising administering to the subject an effective amount of a compound represented by Formula (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C as described herein, or a pharmaceutically acceptable salt of any of the foregoing, or a pharmaceutical composition thereof, wherein the malignant growth or tumor is due to a cancer selected from the list of cancers described herein.

Another embodiment provides a method of ameliorating or treating a malignant growth or a tumor in a subject comprising administering to the subject an effective amount of a compound represented by Formula (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C as described herein, or a pharmaceutically acceptable salt of any of the foregoing, or a pharmaceutical composition thereof, wherein the malignant growth or tumor is due to a cancer selected from the list of cancers described herein.

### DETAILED DESCRIPTION

The membrane-associated tyrosine- and threonine-specific CDC2-inhibitory kinase (PKMYT1) is a member of the WEE1 family and was first reported as a kinase capable of phosphorylated CDC2 efficiently on both threonine-14 and tyrosine-15 in Xenopus. PKMYT1 has been reported to inhibit cell cycle progression by inhibiting the activities of cell cycle-associated proteins, such as Cyclin A, CDK1, and CDK2. However, in recent reports, PKMYT1 was also found to drive the progression of a variety of tumors.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “a substituted or an unsubstituted” or “a substituted or an unsubstituted,” that group may be unsubstituted or substituted with one or more of the specified substituents. If no substituents are specified, it is meant that the group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, 0-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, hydroxyalkyl, haloalkoxy, an amino, a mono-substituted amine group, a di-substituted amine group and an amine(C₁-C₆ alkyl).

As used herein, “Cₐ to C_{b}” and “C_{a-b}” in which “a” and “b” are integers refer to the number of carbon atoms in a group. The indicated group can contain from “a” to “b”, inclusive, carbon atoms. Thus, for example, a “C₁ to C₄ alkyl” and “C₁-C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

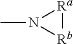

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted. As a non-limiting example, a substituted alkyl group includes haloalkyls and deuterated alkyls. Deuteration (hydrogen to deuterium switch) may improve the solubility as well as pharmacokinetic and/or toxicity profile of drugs which can translate in improvements in efficacy and safety compared with the non-deuterated counterparts. Such efficacy and safety improvements may be observed in, for example, hepatocyte data (e.g., comparing clearance values) and comparing permeability.

The term “heteroalkyl” used herein refers to an alkyl as defined herein, whereby one or more CH₂ groups each can be replaced with a heteroatom, such as, but not limited to, O, S, P(═O)₂ and NH. A heteroalkyl group may be substituted or unsubstituted. As a non-limiting example, a substituted heteroalkyl group includes haloheteroalkyls.

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted. As a non-limiting example, a substituted alkenyl group includes haloalkenyls.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted. As a non-limiting example, a substituted alkynyl group includes haloalkynyls.

As used herein, “cycloalkyl” refers to a completely saturated (no double or triple bonds) mono- or multi-cyclic hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. As a non-limiting example, a substituted cycloalkyl group includes halocycloalkyls. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted. As a non-limiting example, a substituted cycloalkenyl group includes halocycloalkenyls.

As used herein, “carbocyclyl” refers to a non-aromatic a mono- or multi-cyclic hydrocarbon ring system. When composed of two or more rings, the rings may be joined together in a fused, bridged or spiro fashion, as described herein. Carbocyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A carbocyclyl group may be unsubstituted or substituted. As a non-limiting example, a substituted carbocyclyl group includes halocarbocyclyls. Examples of carbocyclyl groups include, but are in no way limited to, cycloalkyl groups and cycloalkenyl groups, as defined herein, and the non-aromatic portions of 1,2,3,4-tetrahydronaphthalene, 2,3-dihydro-1H-indene, 5,6,7,8-tetrahydroquinoline and 6,7-dihydro-5H-cyclopenta[b]pyridine.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group such as phenyl may be substituted or unsubstituted. As a non-limiting example, a substituted aryl group includes haloaryls.

As used herein, “heteroaryl” refers to a monocyclic or multicyclic aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two or more rings, such as at least one aryl ring and at least one heteroaryl ring or at least two heteroaryl rings in a bicyclic system, share at least one chemical bond. The term “heteroaryl” also includes spiro ring systems where two or more rings, such as at least one aryl ring and at least one heteroaryl ring or at least two heteroaryl rings in a bicyclic system, share at least one atom. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted. As a non-limiting example, a substituted heteroaryl group includes haloheteroaryls.

As used herein, “heterocyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common, such as 3-oxabicyclo[3.1.0]hexane. As used herein, the term “bridged heterocyclyl” refers to compounds wherein the heterocyclyl contains a linkage of one or more atoms connecting non-adjacent atoms. As used herein, the term “spiro” refers to two rings which have one atom in common and the two rings are not linked by a bridge. Heterocyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heterocyclyl may be quaternized. Heterocyclyl groups may be unsubstituted or substituted. As a non-limiting example, a substituted heterocyclyl group includes haloheterocyclyls. Examples of “heterocyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isooxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “alkylene groups” are straight-chained alkyl groups that are —CH₂— tethering, forming bonds to connect molecular fragments via their terminal carbon atoms. Embodiments of alkylene groups may be represented herein by —(CH₂)ₙ— where n is an integer in the range of 1 to 30. “Lower” alkylene groups are alkylene groups that contain 1 to 6 carbon atoms. Examples of lower alkylene groups include but are not limited to methylene (—CH₂—), ethylene (—CH₂CH₂—), propylene (—CH₂CH₂CH₂—) and butylene (—CH₂CH₂CH₂CH₂—). An alkylene group can be substituted by replacing one or more hydrogen of the alkylene group and/or by substituting both hydrogens on the same carbon with a cycloalkyl group (e.g.,

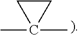

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (iso-propoxy), n-butoxy, iso-butoxy, sec-butoxy, tert-butoxy, phenoxy and benzoxy. An alkoxy may be substituted or unsubstituted. As a non-limiting example, a substituted alkoxy group includes haloalkoxys.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

A “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine. When the name of a group such as alkyl and alkoxy is preceded by “halo” (e.g., haloalkyl, haloalkoxy, etc.), it means that one or more hydrogen atoms of the group is substituted with a halogen (e.g., CF₃, CHF₂, etc.)

A “thiocarbonyl” group refers to a “—C(═S)R” group in which R can be the same as defined with respect to O-carboxy. A thiocarbonyl may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy.

The term “amino” as used herein refers to a —NH₂ group.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. Examples of mono-substituted amino groups include, but are not limited to, —NH(methyl), —NH(phenyl) and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. Examples of di-substituted amino groups include, but are not limited to, —N(methyl)₂, —N(phenyl)(methyl), —N(ethyl)(methyl) and the like.

As used herein, “amine(alkyl)” group refers to an -(alkylene)-NR′R″ radical where R′ and R″ are independently hydrogen or alkyl as defined herein. An amine(alkyl) may be substituted or unsubstituted. Examples of amine(alkyl) groups include, but are not limited to, —CH₂NH(methyl), —CH₂NH(phenyl), —CH₂CH₂NH(methyl), —CH₂CH₂NH(phenyl), —CH₂N(methyl)₂, —CH₂N(phenyl)(methyl), —NCH₂(ethyl)(methyl), —CH₂CH₂N(methyl)₂, —CH₂CH₂N(phenyl)(methyl), —NCH₂CH₂(ethyl)(methyl) and the like.

Where the number of substituents is not specified (e.g., haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For compounds of Formula (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C as described herein, or a pharmaceutically acceptable salt thereof, those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

The terms “PKMYT1 inhibition”, “PKMYT1 inhibitor” and similar terms as used herein refer to inhibiting the activity or function of a PKMYT1 tyrosine- and threonine-specific kinase, e.g., by reducing the activity of PKMYT1 tyrosine- and threonine-specific kinase with regard to mediating phosphorylation of CDK1.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium), hydrogen-2 (deuterium), and hydrogen-3 (tritium). Compounds, conjugates, and immunoconjugates described herein can also include all isotopes of atoms occurring in the intermediates or final products. Isotopes include those atoms having the same atomic number but different mass numbers. For example, isotopes of hydrogen include tritium, deuterium, and tritium.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound, conjugate, or immunoconjugate encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ and the term ‘example’ is used to provide exemplary instances of the item in discussion, not an exhaustive or limiting list thereof. In addition, the term “comprising” is to be interpreted synonymously with the phrases “having at least” or “including at least”. When used in the context of a compound, composition or device, the term “comprising” means that the compound, composition or device includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

In a first embodiment, this disclosure relates to a compound represented by the following Formula (I):

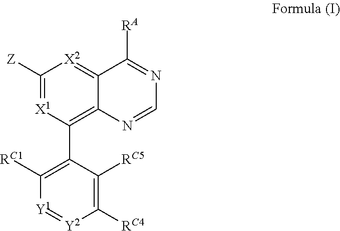

or a pharmaceutically acceptable salt thereof, wherein:
X¹ is —CR^{B1} or N (nitrogen);
X² is —CR^{B2} or N (nitrogen);
Y¹ is —CR^{C2} or N (nitrogen);
Y² is —CR^{C3} or N (nitrogen), provided that Y¹ and Y² are not both N (nitrogen);
Z is

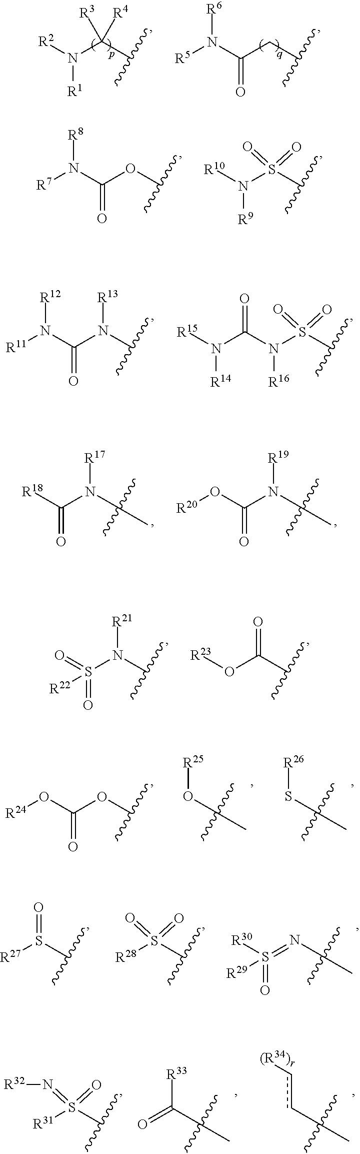

halogen, —CN, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl, wherein:
   [symbol] is a double bond or a triple bond;
   p and q are each independently 0 or 1;
   r is 1 or 2;
   R¹, R², R⁵, R⁶, R⁷, R⁸, R⁹, R¹⁰, R¹¹, R¹², R¹⁴, and R¹⁵ are each independently hydrogen, a substituted or an unsubstituted C₁-C₆ alkyl, a substituted or an unsubstituted C₁-C₆ heteroalkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 4- to 10-membered heterocyclyl, or a substituted or an unsubstituted 5- to 10-membered heteroaryl; or alternatively
   R¹ and R², R⁵ and R⁶, R⁷ and R⁸, R⁹ and R¹⁰, R¹¹ and R¹², or R¹⁴ and R¹⁵, together with the respective nitrogen atom connected thereto, each independently form a substituted or an unsubstituted 4- to 10-membered heterocyclyl, or a substituted or an unsubstituted 5- to 10-membered heteroaryl;
   R³ and R⁴ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; or alternatively
   R³ and R⁴, together with the carbon atom connected thereto, each independently form a substituted or an unsubstituted C₃-C₆ cycloalkyl or a substituted or an unsubstituted 4- to 6-membered heterocyclyl;
   R¹³, R¹⁶, R¹⁷, R¹⁹, and R²¹ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, or a substituted or an unsubstituted C₃-C₆ cycloalkyl;
   R¹⁸, R²⁰, R²³, R²⁴, R²⁵, and R²⁶ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl;
   R²², R²⁷, R²⁸, R²⁹, R³⁰, and R³¹ are each independently a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl;
   R³² is hydrogen, —CN, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl;
   R³³ and R³⁴ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; and
R^{A} is —NR³⁵R³⁶, wherein:
   R³⁵ and R³⁶ are each independently hydrogen, a substituted or an unsubstituted C₁-C₆ alkyl, a substituted or an unsubstituted C₃-C₆ cycloalkyl, or a substituted or an unsubstituted C₄-C₆ heterocyclyl; or alternatively
   one of R³⁵ and R³⁶ is hydrogen or a substituted or an unsubstituted C₁-C₆ alkyl, and the other is a substituted or an unsubstituted 6- or 10-membered aryl or a substituted or an unsubstituted 5- to 10-membered heteroaryl;
R^{B1} and R^{B2} are each independently hydrogen, halogen, —CN, —OH, —OR³⁷, —SR³⁸, a substituted or an unsubstituted C₁-C₆ alkyl, a substituted or an unsubstituted C₁-C₆ heteroalkyl, a substituted or an unsubstituted C₂-C₆ alkenyl, a substituted or an unsubstituted C₂-C₆ alkynyl, a substituted or an unsubstituted C₃-C₆ cycloalkyl, or a substituted or an unsubstituted 5- to 10-membered heteroaryl, wherein:
   R³⁷ and R³⁸, for each instance, are each independently a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, or a substituted or an unsubstituted C₄-C₆ heterocyclyl;
R^{C1}, R^{C2}, and R^{C3} are each independently hydrogen, halogen, —CN, —OH, —OR³⁹, —SR⁴⁰, a substituted or an unsubstituted C₁-C₆ alkyl, a substituted or an unsubstituted C₂-C₆ alkenyl, a substituted or an unsubstituted C₂-C₆ alkynyl, or a substituted or an unsubstituted C₃-C₆ cycloalkyl;
R^{C4} and R^{C5} are each independently hydrogen, halogen, —CN, —OH, —OR³⁹, —SR⁴⁰, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, or a substituted or an unsubstituted C₃-C₆ cycloalkyl; or alternatively
R^{C4} and R^{C5}, together with the carbon atom that each of R^{C4} and R^{C5} is respectively connected thereto, form a 5-membered a substituted or an unsubstituted heteroaryl, wherein:
   R³⁹ and R⁴⁰, for each instance, are each independently a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, or a substituted or an unsubstituted C₄-C₆ heterocyclyl;
provided that R^{C1}, R^{C2}, R^{C3}, R^{C4} and R^{C5} are not all hydrogen (for example, one of R^{C1}, R^{C2}, R^{C3}, R^{C4} and R^{C5} is not hydrogen, two of R^{C1}, R^{C2}, R^{C3}, R^{C4} and R^{C5}, three of R^{C1}, R^{C2}, R^{C3}, R^{C4} and R^{C5} are not hydrogen, four of R^{C1}, R^{C2}, R^{C3}, R^{C4} and R^{C5} or all of R^{A}, R^{B} and R^{C} are not hydrogen); and
with the proviso that the compound represented by Formula (I) is not N-butyl-6-chloro-N-ethyl-8-(2,4,6-trimethylphenyl)-4-quinazolinamine.

In a second embodiment, in a compound represented by Formula (I), or a pharmaceutically acceptable salt thereof, Y¹ is —CR^{C2} and Y² is —CR^{C3}; and all other variables are as defined in any one of the first through hundred-and-fourth embodiments.

In a third embodiment, in a compound represented by Formula (I), or a pharmaceutically acceptable salt thereof, Y¹ is —CR^{C2} and Y² is N; and all other variables are as defined in any one of the first through hundred-and-fourth embodiments.

In a fourth embodiment, in a compound represented by Formula (I), or a pharmaceutically acceptable salt thereof, Y¹ is N and Y² is —CR^{C3}; and all other variables are as defined in any one of the first through hundred-and-fourth embodiments.

In a fifth embodiment, in a compound represented by Formula (I), or a pharmaceutically acceptable salt thereof, the 5-membered heteroaryl formed by R^{C4} and R^{C5}, together with the carbon atom that each of R^{C4} and R^{C5} is respectively connected thereto, is a substituted or an unsubstituted 5-membered heteroaryl containing 1 or 2 nitrogen atoms; and all other variables are as defined in any one of the first through hundred-and-fourth embodiments.

In a sixth embodiment, a compound of this disclosure is represented by the following Formula (Ia), (Ib), (Ic), or (Id):

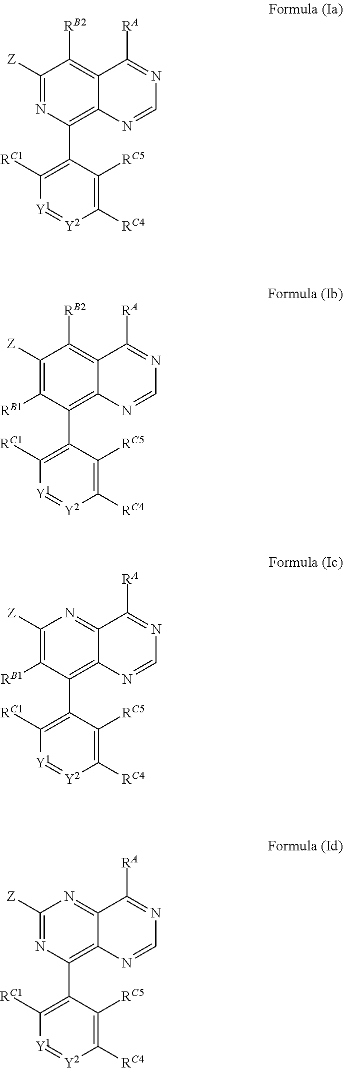

or a pharmaceutically acceptable salt thereof; and all other variables are as defined in any one of the first through hundred-and-fourth embodiments.

In a seventh embodiment, in a compound represented by Formula (I), (Ia), (Ib), (Ic), or (Id), or a pharmaceutically acceptable salt thereof, the substituted or unsubstituted 5-membered heteroaryl formed by R^{C4} and R^{C5}, together with the carbon atom that each of R^{C4} and R^{C5} is respectively connected thereto, is a substituted or an unsubstituted 5-membered heteroaryl containing 1 nitrogen atom; and all other variables are as defined in any one of the first through hundred-and-fourth embodiments.

In an eighth embodiment, in a compound represented by Formula (I), (Ia), (Ib), (Ic), or (Id), or a pharmaceutically acceptable salt thereof, the substituted or unsubstituted 5-membered heteroaryl formed by R^{C4} and R^{C5}, together with the carbon atom that each of R^{C4} and R^{C5} is respectively connected thereto, is a substituted or an unsubstituted 5-membered heteroaryl containing 2 nitrogen atoms; and all other variables are as defined in any one of the first through hundred-and-fourth embodiments.

In a ninth embodiment, in a compound represented by Formula (I), (Ia), (Ib), (Ic), or (Id), or a pharmaceutically acceptable salt thereof, R^{C1}, R^{C2}, R^{C3}, R^{C4} and R^{C5} are each independently hydrogen, halogen, —CN, —OH, —OR³⁹, —SR⁴⁰, a substituted or an unsubstituted C₁-C₆ alkyl, a substituted or an unsubstituted C₂-C₆ alkenyl, a substituted or an unsubstituted C₂-C₆ alkynyl, or a substituted or an unsubstituted C₃-C₆ cycloalkyl; and all other variables are as defined in any one of the first through hundred-and-fourth embodiments.

In a tenth embodiment, in a compound represented by Formula (I), or a pharmaceutically acceptable salt thereof, where X¹ is —CR^{B1}; X² is —CR^{B2}; Y¹ is —CR^{C2}; and Y² is —CR^{C3}, in a compound represented by Formula (Ib), or a pharmaceutically acceptable salt thereof, where Y¹ is —CR^{C2}; Y² is —CR^{C3} and Z is

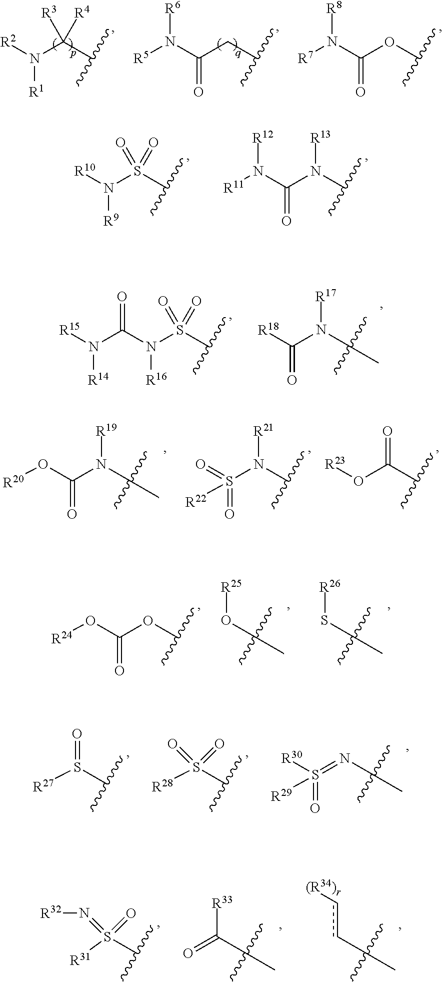

—CN, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; and all other variables are as defined in any one of the first through hundred-and-fourth embodiments.

In an eleventh embodiment, a compound of this disclosure is represented by the following Formula (II) or (II-1):

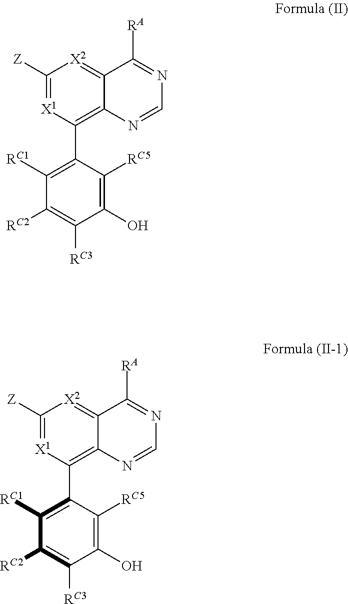

or a pharmaceutically acceptable salt thereof; and all other variables are as defined in any one of the first through hundred-and-fourth embodiments.

In a twelfth embodiment, in a compound represented by Formula (I), (Ia), (Ib), (Ic), (Id), (II), or (II-1), or a pharmaceutically acceptable salt thereof, R³⁵ and R³⁶ are each independently hydrogen, a substituted or an unsubstituted C₁-C₆ alkyl, a substituted or an unsubstituted C₃-C₆ cycloalkyl, or a substituted or an unsubstituted C₄-C₆ heterocyclyl; and all other variables are as defined any one of the first through hundred-and-fourth embodiments.

In a thirteenth embodiment, in a compound represented by Formula (I), (Ia), (Ib), (Ic), (Id), (II), or (II-1), or a pharmaceutically acceptable salt thereof, R³⁵ and R³⁶ are each independently hydrogen, a substituted or an unsubstituted C₁-C₆ alkyl, or a substituted or an unsubstituted C₃-C₆ cycloalkyl; and all other variables are as defined any one of the first through hundred-and-fourth embodiments.

In a fourteenth embodiment, in a compound represented by Formula (I), (Ia), (Ib), (Ic), (Id), (II), or (II-1), or a pharmaceutically acceptable salt thereof, R³⁵ and R³⁶ are each independently hydrogen or a substituted or an unsubstituted C₁-C₆ alkyl; or R³⁵ and R³⁶ are each independently hydrogen or a substituted or an unsubstituted C₁-C₄ alkyl; or R³⁵ and R³⁶ are each independently hydrogen or a substituted or an unsubstituted C₁-C₃ alkyl; or R³⁵ and R³⁶ are each independently hydrogen or a substituted or an unsubstituted C₁-C₂ alkyl; and all other variables are as defined any one of the first through hundred-and-fourth embodiments.

In a fifteenth embodiment, a compound of this disclosure is represented by the following Formula (IIIa), (IIIb), (IIIc), (IIId), (IIIa-1), (IIIb-1), (IIIc-1), or (IIId-1):

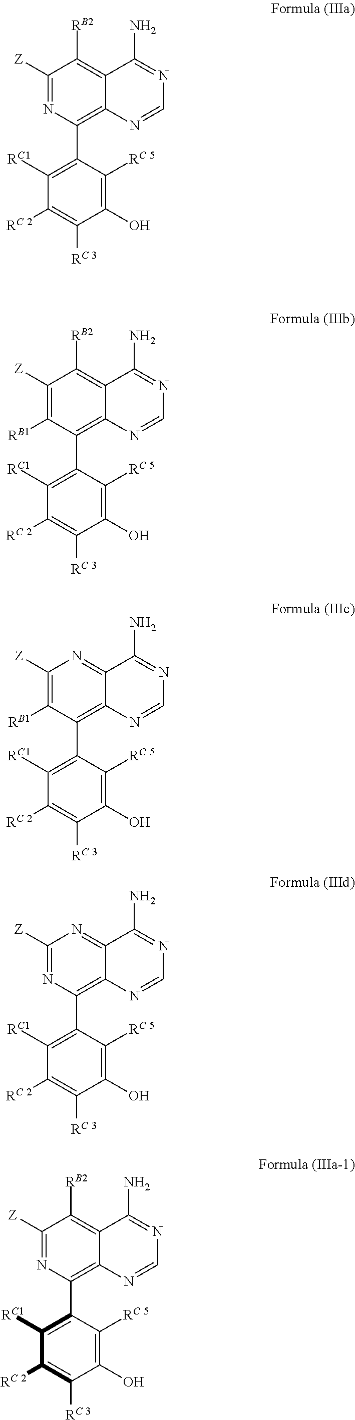

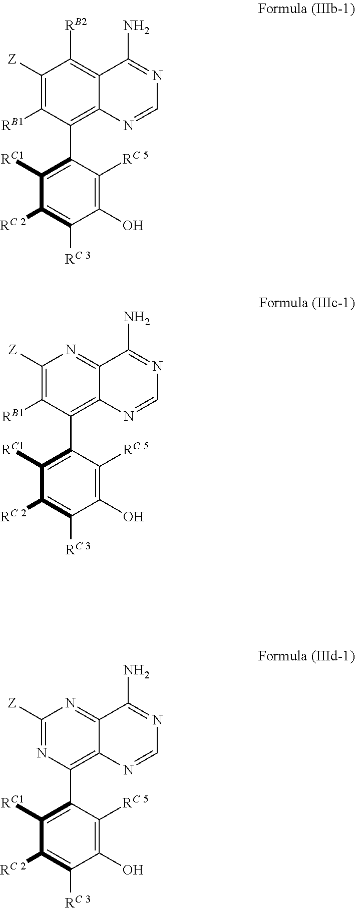

or a pharmaceutically acceptable salt thereof, and all other variables are as defined in any one of the first through hundred-and-fourth embodiments.

In a sixteenth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1) or a pharmaceutically acceptable salt thereof, R^{C1}, R^{C2}, R^{C3} and R^{C5} are each independently hydrogen, halogen, —CN, —OH, —OR³⁹, a substituted or an unsubstituted C₁-C₆ alkyl, or a substituted or an unsubstituted C₃-C₆ cycloalkyl; wherein R³⁹ is a substituted or an unsubstituted C₁-C₆ alkyl or a substituted or an unsubstituted C₃-C₆ cycloalkyl; and all other variables are as defined in any one of the first through hundred-and-fourth embodiments.

In a seventeenth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R^{C1}, R^{C2}, R^{C3} and R^{C5} are each independently hydrogen, halogen, —CN, —OH, —OR³⁹, a substituted or an unsubstituted C₁-C₆ alkyl or a substituted or an unsubstituted C₃-C₆ cycloalkyl; wherein R³⁹ is a substituted or an unsubstituted C₁-C₆ alkyl (e.g., a substituted or an unsubstituted C₁-C₆ alkyl, a substituted or an unsubstituted C₁-C₄ alkyl, a substituted or an unsubstituted C₁-C₃ alkyl, or a substituted or an unsubstituted C₁-C₂ alkyl, etc.); and all other variables are as defined in any one of the first through hundred-and-fourth embodiments.

In an eighteenth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R^{C1}, R^{C2}, R^{C3} and R^{C5} are each independently hydrogen, F, Cl, Br, —CN, or a substituted or an unsubstituted C₁-C₆ alkyl; and all other variables are as defined in any one of the first through hundred-and-fourth embodiments.

In a nineteenth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R^{C1}, R^{C2}, R^{C3} and R^{C5} are each independently hydrogen, Cl, methyl, —CF₃, or —CHF₂; and all other variables are as defined in any one of the first through hundred-and-fourth embodiments.

In a twentieth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), (IIId-1), (IVa), (IVa-1), (Va), or (Va-1), or a pharmaceutically acceptable salt thereof, at least one of R^{C1}, R^{C2}, R^{C3} and R^{C5} is not hydrogen; and all other variables are as defined in any one of the first through hundred-and-fourth embodiments.

In a twenty-first embodiment, in a compound represented by Formula (I), (Ia), (Ib), (Ic), (Id), (II), (II-1) or a pharmaceutically acceptable salt thereof, at least two of R^{C1}, R^{C2}, R^{C3} and R^{C5} are not hydrogen; and all other variables are as defined in any one of the first through hundred-and-fourth embodiments.

In a twenty-second embodiment, in a compound represented by Formula (I), (Ia), (Ib), (Ic), (Id), (II), or (II-1), or a pharmaceutically acceptable salt thereof, R^{B1} and R^{B2} are each independently hydrogen, halogen, —CN, —OH, —OR³⁷, a substituted or an unsubstituted C₁-C₆ alkyl, a substituted or an unsubstituted C₁-C₆ heteroalkyl, or a substituted or an unsubstituted C₃-C₆ cycloalkyl; wherein R³⁷ is a substituted or an unsubstituted C₁-C₆ alkyl or a substituted or an unsubstituted C₃-C₆ cycloalkyl; and all other variables are as defined in any one of the first through hundred-and-fourth embodiments.

In a twenty-third embodiment, in a compound represented by Formula (I), (Ia), (Ib), (Ic), (Id), (II), or (II-1), or a pharmaceutically acceptable salt thereof, R^{B1} and R^{B2} are each independently hydrogen, F, Cl, Br, —CN, —OR³⁷, or a substituted or an unsubstituted C₁-C₆ alkyl; wherein R³⁷ is a substituted or an unsubstituted C₁-C₆ alkyl; and all other variables are as defined in any one of the first through hundred-and-fourth embodiments.

In a twenty-fourth embodiment, in a compound represented by Formula (I), (Ia), (Ib), (Ic), (Id), (II), or (II-1), or a pharmaceutically acceptable salt thereof, R^{B1} is each independently hydrogen, F, Cl, —CN, —OR³⁷, or a substituted or an unsubstituted C₁-C₆ alkyl; wherein R³⁷ is a substituted or an unsubstituted C₁-C₄ alkyl; and all other variables are as defined in any one of the first through hundred-and-fourth embodiments.

In a twenty-fifth embodiment, in a compound represented by Formula (I), (Ia), (Ib), (Ic), (Id), (II), or (II-1), or a pharmaceutically acceptable salt thereof, R^{B2} is hydrogen, F, methyl, or methoxy; and all other variables are as defined in any one of the first through hundred-and-fourth embodiments.

In a twenty-sixth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1) or a pharmaceutically acceptable salt thereof, Z is

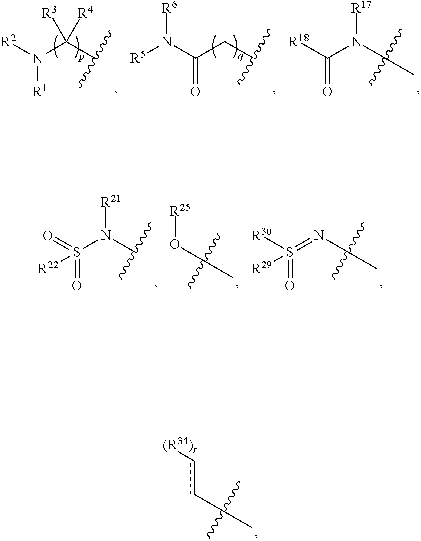

—CN, a substituted or an unsubstituted C₁-C₆ alkyl, a substituted or an unsubstituted C₂-C₆ alkenyl, a substituted or an unsubstituted C₃-C₆ cycloalkyl, a substituted or an unsubstituted phenyl, a substituted or an unsubstituted 4- to 10-membered heterocyclyl, or a substituted or an unsubstituted 5- to 10-membered heteroaryl; and all other variables are as defined in any one of the first through hundred-and-fourth embodiments.

In a twenty-seventh embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IId-1) or a pharmaceutically acceptable salt thereof, Z is

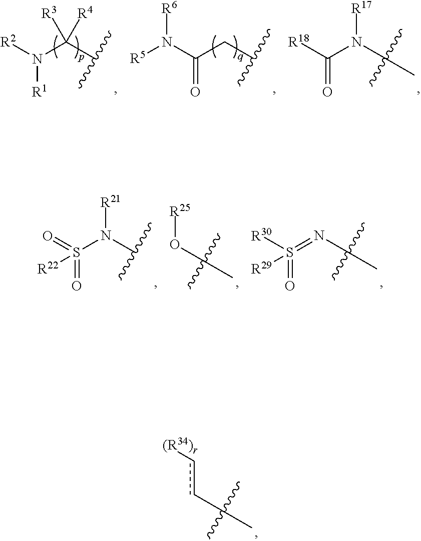

—CN, a substituted or an unsubstituted C₁-C₆ alkyl, a substituted or an unsubstituted C₃-C₆ cycloalkyl, a substituted or an unsubstituted phenyl, or a substituted or an unsubstituted 5- to 10-membered heteroaryl; and all other variables are as defined in any one of the first through hundred-and-fourth embodiments.

In a twenty-eighth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

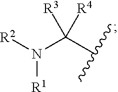

and all other variables are as defined in any one of the first through hundred-and-fourth embodiments.

In a twenty-ninth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R³ and R⁴ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; and all other variables are as defined in any one of the first through hundred-and-fourth embodiments.

In a thirtieth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R³ and R⁴ are each independently hydrogen, a substituted or an unsubstituted C₁-C₆ alkyl, or a substituted or an unsubstituted C₃-C₆ cycloalkyl; and all other variables are as defined in any one of the first through hundred-and-fourth embodiments.

In a thirty-first embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R³ and R⁴ are each independently hydrogen or a substituted or an unsubstituted C₁-C₄ alkyl; and all other variables are as defined in any one of the first through hundred-and-fourth embodiments.

In a thirty-second embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R¹, R², R⁵, R⁶, R⁷, R⁸, R⁹, R¹⁰, R¹¹, R¹², R¹⁴, and R¹⁵ are each independently hydrogen, a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁-C₂ alkyl, —CD₃, etc.), a substituted or an unsubstituted C₁-C₆ heteroalkyl (a substituted or an unsubstituted C₁-C₆ heteroalkyl, a substituted or an unsubstituted C₁-C₄ heteroalkyl, a substituted or an unsubstituted C₁-C₃ heteroalkyl, a substituted or an unsubstituted C₁-C₂ heteroalkyl, etc.), a substituted or an unsubstituted C₁-C₆ alkoxy, a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.), a substituted or an unsubstituted 4- to 10-membered heterocyclyl (a substituted or an unsubstituted 4- to 10-membered heterocyclyl, a substituted or an unsubstituted 4- to 8-membered heterocyclyl, a substituted or an unsubstituted 5- to 8-membered heterocyclyl, a substituted or an unsubstituted 4- to 6-membered heterocyclyl, etc.), or a substituted or an unsubstituted 5- to 10-membered heteroaryl (a substituted or an unsubstituted 5- to 10-membered heteroaryl, a substituted or an unsubstituted 5- to 8-membered heteroaryl, a substituted or an unsubstituted 5- or 6-membered heteroaryl, a substituted or an unsubstituted 5-membered heteroaryl, a substituted or an unsubstituted 6-membered heteroaryl, etc.), wherein:
when a C₁-C₆ alkyl or a C₁-C₆ heteroalkyl represented by R¹, R², R⁵, R⁶, R⁷, R⁸, R⁹, R¹⁰, R¹¹, R¹², R¹⁴, or R¹⁵ is substituted, the group is substituted with one or more groups each independently selected from halogen, —CN, —OH, C₁-C₆ alkyl, C₁-C₆ haloalkyl, C₁-C₆ alkoxy, and C₃-C₆ cycloalkyl;
when a C₃-C₆ cycloalkyl, a 4- to 10-membered heterocyclyl, or a 5- to 10-membered heteroaryl represented by R¹, R², R⁵, R⁶, R⁷, R⁸, R⁹, R⁰, R¹¹, R¹², R¹⁴, or R¹⁵ is substituted, the group is substituted with one or more groups each independently selected from halogen, —CN, —OH, C₁-C₆ alkyl, C₁-C₆ alkyl substituted with one or more groups each independently selected from C₁-C₄ alkoxy, C₁-C₆ haloalkyl, and C₁-C₆ alkoxy; or alternatively
R¹ and R², R⁵ and R⁶, R⁷ and R⁸, R⁹ and R¹⁰, R¹¹ and R¹², or R¹⁴ and R¹⁵, together with the respective nitrogen atom connected thereto, each independently form a substituted or an unsubstituted 4- to 10-membered heterocyclyl or a substituted or an unsubstituted 5- to 10-membered heteroaryl, wherein:
   when a 4- to 10-membered heterocyclyl or a 5- to 10-membered heteroaryl formed by R¹ or R², R⁵ and R⁶, R⁷ and R⁸, R⁹ and R¹⁰, R¹¹ and R¹², or R¹⁴ and R¹⁵, together with the respective nitrogen atom connected thereto, is substituted, the group is substituted with one or more groups each independently selected from halogen, —CN, —OH, C₁-C₆ alkyl, C₁-C₆ alkyl substituted with one or more groups each independently selected from —OH, C₁-C₄ alkoxy, C₁-C₆ haloalkyl, and C₁-C₆ alkoxy;
and all other variables are as defined in any one of the first through hundred-and-fourth embodiments.

In a thirty-third embodiment, a compound of this disclosure is represented by the following Formula (IVa) or (IVa-1):

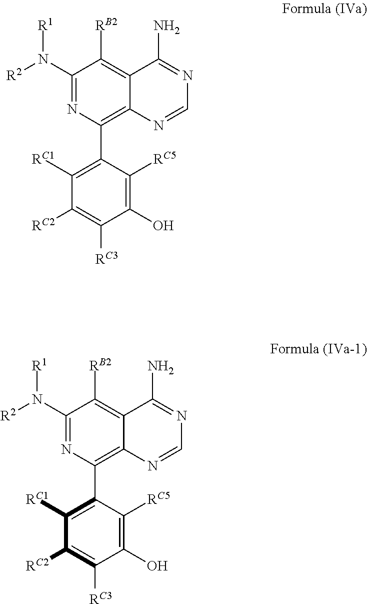

or a pharmaceutically acceptable salt thereof, and all other variables are as defined in any one of the first through hundred-and-fourth embodiments.

In a thirty-fourth embodiment, a compound of this disclosure is represented by the following Formula (Va) or (Va-1):

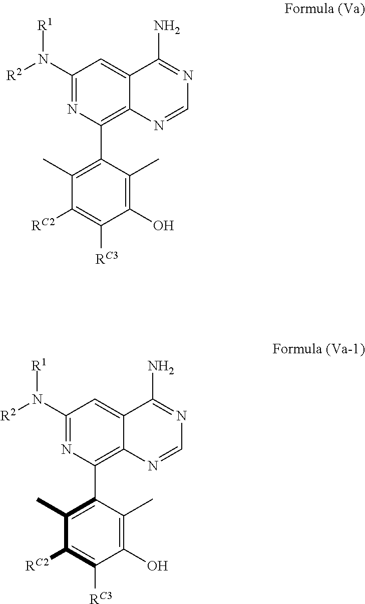

or a pharmaceutically acceptable salt thereof, and all other variables are as defined in any one of the first through hundred-and-fourth embodiments.

In a thirty-fifth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), (IIId-1), (IVa), (IVa-1), (Va), or (Va-1), or a pharmaceutically acceptable salt thereof, R¹ and R² are each independently hydrogen, a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁-C₂ alkyl, —CD₃, etc.), a substituted or an unsubstituted C₁-C₆ heteroalkyl (a substituted or an unsubstituted C₁-C₆ heteroalkyl, a substituted or an unsubstituted C₁-C₄ heteroalkyl, a substituted or an unsubstituted C₁-C₃ heteroalkyl, a substituted or an unsubstituted C₁-C₂ heteroalkyl, etc.), a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.), a substituted or an unsubstituted 4- to 10-membered heterocyclyl (a substituted or an unsubstituted 4- to 10-membered heterocyclyl, a substituted or an unsubstituted 4- to 8-membered heterocyclyl, a substituted or an unsubstituted 5- to 8-membered heterocyclyl, a substituted or an unsubstituted 4- to 6-membered heterocyclyl, etc., including a substituted or an unsubstituted fused bicyclic 6-membered heterocyclyl containing at least one oxygen atom), or a substituted or an unsubstituted 5- to 10-membered heteroaryl (a substituted or an unsubstituted 5- to 10-membered heteroaryl, a substituted or an unsubstituted 5- to 8-membered heteroaryl, a substituted or an unsubstituted 5- or 6-membered heteroaryl, a substituted or an unsubstituted 5-membered heteroaryl, a substituted or an unsubstituted 6-membered heteroaryl, etc.), wherein:
when a C₁-C₆ alkyl or a C₁-C₆ heteroalkyl represented by R¹ or R² is substituted, the C₁-C₆ alkyl or the C₁-C₆ heteroalkyl is substituted with one or more groups each independently selected from halogen, —CN, —OH, C₁-C₄ alkyl, C₁-C₄ haloalkyl, C₁-C₄ alkoxy, and C₃-C₆ cycloalkyl;
when a C₃-C₆ cycloalkyl, a 4- to 10-membered heterocyclyl, or a 5- to 10-membered heteroaryl represented by R¹ or R² is substituted, the C₃-C₆ cycloalkyl, the 4- to 10-membered heterocyclyl, or the 5- to 10-membered heteroaryl is substituted with one or more groups each independently selected from halogen, —CN, —OH, C₁-C₄ alkyl, C₁-C₄ alkyl substituted with one or more groups each independently selected from C₁-C₂ alkoxy, C₁-C₄ haloalkyl, and C₁-C₄ alkoxy; or alternatively
R¹ and R², together with the nitrogen atom connected thereto, form a substituted or an unsubstituted 4- to 8-membered heterocyclyl (a substituted or an unsubstituted 4- to 8-membered heterocyclyl, a substituted or an unsubstituted 5- to 8-membered heterocyclyl, a substituted or an unsubstituted 4- to 6-membered heterocyclyl, etc.) or a substituted or an unsubstituted 5- to 8-membered heteroaryl (a substituted or an unsubstituted 5- to 8-membered heteroaryl, a substituted or an unsubstituted 5- or 6-membered heteroaryl, a substituted or an unsubstituted 5-membered heteroaryl, or a substituted or an unsubstituted 6-membered heteroaryl, etc.), wherein:
when a 4- to 8-membered heterocyclyl or a 5- to 8-membered heteroaryl formed by R¹ or R², together with the nitrogen atom connected thereto, is substituted, the 4- to 8-membered heterocyclyl or the 5- to 8-membered heteroaryl is substituted with one or more groups each independently selected from halogen, —CN, —OH, C₁-C₄ alkyl, C₁-C₄ alkyl substituted with one or more groups each independently selected from —OH, C₁-C₂ alkoxy, C₁-C₄ haloalkyl, and C₁-C₄ alkoxy;
and all other variables are as defined in any one of the first through hundred-and-fourth embodiments.

In a thirty-sixth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), (IIId-1), (IVa), (IVa-1), (Va), or (Va-1), or a pharmaceutically acceptable salt thereof, R¹ and R² are each independently hydrogen, a substituted or an unsubstituted C₁-C₄ alkyl, a substituted or an unsubstituted C₃-C₅ cycloalkyl, a substituted or an unsubstituted 4- to 8-membered heterocyclyl (e.g., a substituted or an unsubstituted fused bicyclic 6-membered heterocyclyl containing at least one oxygen atom), or a substituted or an unsubstituted 5- to 8-membered heteroaryl, wherein:
when a C₁-C₄ alkyl represented by R¹ or R² is substituted, the C₁-C₄ alkyl is substituted with one or more groups each independently selected from F, Cl, —CN, —OH, C₁-C₂ alkyl, C₁-C₂ haloalkyl, C₁-C₂ alkoxy, and C₃-C₄ cycloalkyl;
when a C₃-C₅ cycloalkyl, a 4- to 8-membered heterocyclyl, or a 5- to 8-membered heteroaryl represented by R¹ or R² is substituted, the C₃-C₅ cycloalkyl, the 4- to 8-membered heterocyclyl, or the 5- to 8-membered heteroaryl is substituted with one or more groups each independently selected from F, Cl, —CN, —OH, C₁-C₂ alkyl, C₁-C₂ alkyl substituted one or more groups each independently selected from with C₁-C₂ alkoxy, C₁-C₂ haloalkyl, and C₁-C₂ alkoxy; or alternatively
R¹ and R², together with the nitrogen atom connected thereto, form a substituted or an unsubstituted 4- to 8-membered heterocyclyl or a substituted or an unsubstituted 5- or 6-membered heteroaryl, wherein:
when a 4- to 8-membered heterocyclyl or a 5- or 6-membered heteroaryl formed by R¹ or R², together with the nitrogen atom connected thereto, is substituted, the 4- to 8-membered heterocyclyl or the 5- or 6-membered heteroaryl is substituted with one or more groups each independently selected from F, Cl, —CN, —OH, C₁-C₂ alkyl, C₁-C₂ alkyl substituted with one or more groups each independently selected from —OH, C₁-C₂ alkoxy, C₁-C₂ haloalkyl, and C₁-C₂ alkoxy;
and all other variables are as defined in any one of the first through hundred-and-fourth embodiments. In some embodiments of the thirty-sixth embodiment, R¹ can be H or an unsubstituted C₁-C₄ alkyl; and R can be an unsubstituted C₁-C₄ alkyl or an unsubstituted C₃-C₅ cycloalkyl. In some embodiments of the thirty-sixth embodiment, R¹ can be H or an unsubstituted C₁-C₄ alkyl; and R² can be an substituted C₁-C₄ alkyl substituted with one or more (e.g., 1, 2 or 3) groups each independently selected from F, —CN, —OH and C₃-C₄ cycloalkyl, a substituted C₃-C₅ cycloalkyl substituted with one or more (e.g., 1, 2 or 3) groups each independently selected from —OH, F and C₁-C₂ alkoxy, a substituted or an unsubstituted 4- to 8-membered heterocyclyl (e.g., a substituted or an unsubstituted fused bicyclic 6-membered heterocyclyl containing at least one oxygen atom such as 3-oxabicyclo[3.1.0]hexane) or a substituted or an unsubstituted 5- to 6-membered monocyclic heteroaryl (such as a substituted or an unsubstituted pyrrolyl, including a C₁-C₂ alkyl substituted pyrrolyl). In some embodiments of the thirty-sixth embodiment, R¹ and R², together with the nitrogen atom connected thereto, form a substituted or an unsubstituted 4- to 8-membered heterocyclyl (e.g., a substituted or an unsubstituted 4- to 8-membered spiro-connected heterocyclyl including a substituted or an unsubstituted 4- to 8-membered spiro-connected heterocyclyl containing at least one oxygen atom such as 1-oxa-6-azaspiro[3.4]octane).

In a thirty-seventh embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), (IIId-1), (IVa), (IVa-1), (Va), or (Va-1), or a pharmaceutically acceptable salt thereof, R¹ and R², together with the nitrogen atom connected thereto, form a substituted or an unsubstituted 5- to 6-membered heterocyclyl (e.g., a substituted or an unsubstituted 5-membered heterocyclyl containing at least one nitrogen atom or a substituted or an unsubstituted 6-membered heterocyclyl containing at least one nitrogen atom and/or at least one oxygen atom and optionally at least one double bond in the heterocyclyl) or a substituted or an unsubstituted 5- or 6-membered heteroaryl, wherein:
when a 5- to 6-membered heterocyclyl or a 5- or 6-membered heteroaryl formed by R¹ or R², together with the nitrogen atom connected thereto, is substituted, the 5- to 6-membered heterocyclyl or the 5- or 6-membered heteroaryl is substituted with one or more groups each independently selected from F, Cl, —CN, —OH, C₁-C₂ alkyl, C₁-C₂ alkyl substituted with one or more groups each independently selected from —OH, C₁-C₂ alkoxy, C₁-C₂ haloalkyl, and C₁-C₂ alkoxy;
and all other variables are as defined in any one of the first through hundred-and-fourth embodiments. In some embodiments of the thirty-seventh embodiment, R¹ and R², together with the nitrogen atom connected thereto, form a substituted or an unsubstituted 5- to 6-membered monocyclic heterocyclyl (such as azetidinyl, pyrrolidinyl, pyrrolidin-2-one, piperidinyl and morpholinyl), wherein the substituted 5- to 6-membered monocyclic heterocyclyl is substituted with one or more groups (e.g., 1, 2 or 3) each independently selected from F, —CN, —OH, C₁-C₂ alkyl, C₁-C₂ alkyl substituted with one or more groups each independently selected from —OH, C₁-C₂ alkoxy, C₁-C₂ haloalkyl, and C₁-C₂ alkoxy.

In a thirty-eighth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

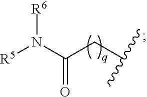

and all other variables are as defined in any one of the first through hundred-and-fourth embodiments.

In a thirty-ninth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

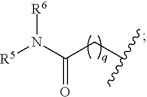

and all other variables are as defined in any one of the first through hundred-and-fourth embodiments. In some embodiments of the forty-second embodiment, R⁵ and R⁶, together with the nitrogen atom connected thereto, form a substituted or an unsubstituted 4- to 6-membered monocyclic heterocyclyl (for example, a substituted or an unsubstituted pyrrolidinyl and a substituted or an unsubstituted morpholinyl). In some embodiments of the forty-second embodiment, R⁵ and R⁶, together with the nitrogen atom connected thereto, form an unsubstituted 4- to 6-membered monocyclic heterocyclyl (for example, an unsubstituted pyrrolidinyl). In some embodiments of the forty-second embodiment, q is 1.

In a fortieth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

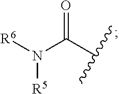

and all other variables are as defined in any one of the first through hundred-and-fourth embodiments. In some embodiments of the forty-second embodiment, R⁵ and R⁶, together with the nitrogen atom connected thereto, form a substituted or an unsubstituted 4- to 6-membered monocyclic heterocyclyl (for example, a substituted or an unsubstituted pyrrolidinyl and a substituted or an unsubstituted morpholinyl). In some embodiments of the forty-second embodiment, R⁵ and R⁶, together with the nitrogen atom connected thereto, form an unsubstituted 4- to 6-membered monocyclic heterocyclyl (for example, an unsubstituted pyrrolidinyl).

In a forty-first embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R⁵ and R⁶ are each independently hydrogen or a substituted or an unsubstituted C₁-C₆ alkyl, wherein:
when a C₁-C₆ alkyl represented by R⁵ or R⁶ is substituted, the C₁-C₆ alkyl is substituted with one or more groups each independently selected from halogen, —CN, —OH, and C₁-C₄ alkoxy; or alternatively
R⁵ and R⁶, together with the nitrogen atom connected thereto, form a substituted or an unsubstituted 4- to 8-membered heterocyclyl, wherein:
when a 4- to 8-membered heterocyclyl formed by R⁵ or R⁶, together with the nitrogen atom connected thereto, is substituted, the 4- to 8-membered heterocyclyl is substituted with one or more groups each independently selected from halogen, —CN, —OH, C₁-C₄ alkyl, C₁-C₄ alkyl substituted with one or more groups each independently selected from —OH, C₁-C₂ alkoxy, C₁-C₄ haloalkyl, and C₁-C₄ alkoxy;
and all other variables are as defined in any one of the first through hundred-and-fourth embodiments.

In a forty-second embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R⁵ and R⁶ are each independently hydrogen or a substituted or an unsubstituted C₁-C₄ alkyl, wherein:
when a C₁-C₄ alkyl represented by R⁵ or R⁶ is substituted, the C₁-C₄ alkyl is substituted with one or more groups each independently selected from F, Cl, —CN, —OH, and C₁-C₂ alkoxy; or alternatively
R⁵ and R⁶, together with the nitrogen atom connected thereto, form a substituted or an unsubstituted 4- to 6-membered heterocyclyl, wherein:
when a 4- to 6-membered heterocyclyl formed by R⁵ or R⁶, together with the nitrogen atom connected thereto, is substituted, the 4- to 6-membered heterocyclyl is substituted with one or more groups each independently selected from F, Cl, —CN, —OH, C₁-C₂ alkyl, C₁-C₂ alkyl substituted with one or more groups each independently selected from —OH, C₁-C₂ alkoxy, C₁-C₂ haloalkyl, and C₁-C₂ alkoxy;
and all other variables are as defined in any one of the first through hundred-and-fourth embodiments.

In a forty-third embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

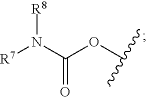

and all other variables are as defined in any one of the first through hundred-and-fourth embodiments.

In a forty-fourth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

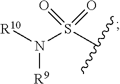

and all other variables are as defined in any one of the first through hundred-and-fourth embodiments.

In a forty-fifth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R¹⁵, R¹⁶, R¹⁷, R¹⁹, and R²¹ are each independently hydrogen, a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁-C₂ alkyl, —CD₃, etc.), a substituted or an unsubstituted C₁-C₆ alkoxy (a substituted or an unsubstituted C₁-C₆ alkoxy, a substituted or an unsubstituted C₁-C₄ alkoxy, a substituted or an unsubstituted C₁-C₃ alkoxy, a substituted or an unsubstituted C₁-C₂ alkoxy, etc.), or a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.); and all other variables are as defined in any one of the first through hundred-and-fourth embodiments.

In a forty-sixth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

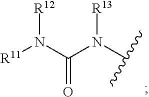

and all other variables are as defined in any one of the first through hundred-and-fourth embodiments.

In a forty-seventh embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

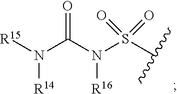

and all other variables are as defined in any one of the first through hundred-and-fourth embodiments.

In a forty-eighth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R¹⁸, R²⁰, R²³, R²⁴, R²⁵, and R²⁶ are each independently hydrogen, a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁-C₂ alkyl, —CD₃, etc.), or a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.); and all other variables are as defined in any one of the first through hundred-and-fourth embodiments.

In a forty-ninth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

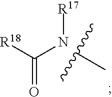

and all other variables are as defined in any one of the first through hundred-and-fourth embodiments.

In a fiftieth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R¹⁷ is hydrogen or a substituted or an unsubstituted C₁-C₆ alkyl; and R¹⁸ is a substituted or an unsubstituted C₁-C₆ alkyl, wherein: when a C₁-C₆ alkyl represented by R¹⁷ or R¹⁸ is substituted, the C₁-C₆ alkyl is substituted with one or more groups each independently selected from halogen, —CN, —OH, and C₁-C₄ alkoxy;
and all other variables are as defined in any one of the first through hundred-and-fourth embodiments.

In a fifty-first embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R¹⁷ is hydrogen or a substituted or an unsubstituted C₁-C₄ alkyl; and R¹⁸ is a substituted or an unsubstituted C₁-C₄ alkyl, wherein: when a C₁-C₄ alkyl represented by R¹⁷ or R¹⁸ is substituted, the C₁-C₄ alkyl is substituted with one or more groups each independently selected from F, Cl, —CN, —OH, and C₁-C₂ alkoxy;
and all other variables are as defined in any one of the first through hundred-and-fourth embodiments.

In a fifty-second embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

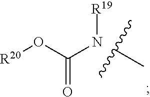

and all other variables are as defined in any one of the first through hundred-and-fourth embodiments.

In a fifty-third embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R²², R²⁷, R²⁸, R²⁹, R³⁰, and R³¹ are each independently a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁-C₂ alkyl, —CD₃, etc.), a substituted or an unsubstituted C₁-C₆ alkoxy (a substituted or an unsubstituted C₁-C₆ alkoxy, a substituted or an unsubstituted C₁-C₄ alkoxy, a substituted or an unsubstituted C₁-C₃ alkoxy, a substituted or an unsubstituted C₁-C₂ alkoxy, etc.), or a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.); and all other variables are as defined in any one of the first through hundred-and-fourth embodiments.

In a fifty-fourth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

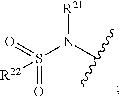

and all other variables are as defined in any one of the first through hundred-and-fourth embodiments.

In a fifty-fifth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R²¹ is hydrogen or a substituted or an unsubstituted C₁-C₆ alkyl; and R²² is a substituted or an unsubstituted C₁-C₆ alkyl, wherein: when a C₁-C₆ alkyl represented by R²¹ or R²² is substituted, the C₁-C₆ alkyl is substituted with one or more groups each independently selected from halogen, —CN, —OH, and C₁-C₄ alkoxy;
and all other variables are as defined in any one of the first through hundred-and-fourth embodiments.

In a fifty-sixth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R²¹ is hydrogen or a substituted or an unsubstituted C₁-C₄ alkyl; and R²² is a substituted or an unsubstituted C₁-C₄ alkyl, wherein: when a C₁-C₄ alkyl represented by R²¹ or R²² is substituted, the C₁-C₄ alkyl is substituted with one or more groups each independently selected from F, Cl, —CN, —OH, and C₁-C₂ alkoxy;
and all other variables are as defined in any one of the first through hundred-and-fourth embodiments. In some embodiments of the fifty-sixth embodiment, R²¹ is hydrogen; and R²² is an unsubstituted C₁-C₄ alkyl (e.g., methyl).

In a fifty-seventh embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

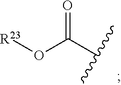

and all other variables are as defined in any one of the first through hundred-and-fourth embodiments.

In a fifty-eighth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

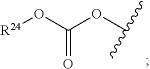

and all other variables are as defined in any one of the first through hundred-and-fourth embodiments.

In a fifty-ninth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

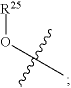

and all other variables are as defined in any one of the first through hundred-and-fourth embodiments.

In a sixtieth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R²⁵ is a substituted or an unsubstituted C₁-C₆ alkyl, wherein:
when a C₁-C₆ alkyl represented by R²⁵ is substituted, the C₁-C₆ alkyl is substituted with one or more groups each independently selected from halogen, —CN, —OH, and C₁-C₄ alkoxy;
and all other variables are as defined in any one of the first through hundred-and-fourth embodiments.

In a sixty-first embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R²⁵ is a substituted or an unsubstituted C₁-C₄ alkyl, wherein:
when a C₁-C₄ alkyl represented by R²⁵ is substituted, the C₁-C₄ alkyl is substituted with one or more groups each independently selected from F, Cl, —CN, —OH, and C₁-C₂ alkoxy;
and all other variables are as defined in any one of the first through hundred-and-fourth embodiments. In some embodiments of the sixty-first embodiment, R² is an unsubstituted C₁-C₄ alkyl (e.g., methyl).

In a sixty-second embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

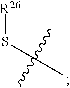

and all other variables are as defined in any one of the first through hundred-and-fourth embodiments.

In a sixty-third embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

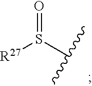

and all other variables are as defined in any one of the first through hundred-and-fourth embodiments.

In a sixty-fourth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

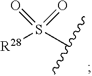

and all other variables are as defined in any one of the first through hundred-and-fourth embodiments.

In a sixty-fifth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

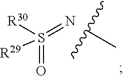

and all other variables are as defined in any one of the first through hundred-and-fourth embodiments.

In a sixty-sixth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R²⁹ and R³⁰ are each a substituted or an unsubstituted C₁-C₆ alkyl, wherein:
when a C₁-C₆ alkyl represented by R²⁹ or R³⁰ is substituted, the C₁-C₆ alkyl is substituted with one or more groups each independently selected from halogen, —CN, —OH, and C₁-C₄ alkoxy;
and all other variables are as defined in any one of the first through hundred-and-fourth embodiments.

In a sixty-seventh embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R²⁹ and R³⁰ are each a substituted or an unsubstituted C₁-C₄ alkyl, wherein:
when a C₁-C₄ alkyl represented by R²⁹ or R³⁰ is substituted, the C₁-C₄ alkyl is substituted with one or more groups each independently selected from F, Cl, —CN, —OH, and C₁-C₂ alkoxy;
and all other variables are as defined in any one of the first through hundred-and-fourth embodiments. In some embodiments of the sixty-seventh embodiment, R²⁹ and R³⁰ are each an unsubstituted C₁-C₄ alkyl (e.g., methyl).

In a sixty-eighth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R³² is hydrogen, —CN, a substituted or an unsubstituted C₁-C₆ alkyl, a substituted or an unsubstituted C₁-C₆ alkoxy, or a substituted or an unsubstituted C₃-C₆ cycloalkyl; and all other variables are as defined in any one of the first through hundred-and-fourth embodiments.

In a sixty-ninth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R³³ and R³⁴ are each independently hydrogen, a substituted or an unsubstituted C₁-C₆ alkyl, a substituted or an unsubstituted C₁-C₆ alkoxy, or a substituted or an unsubstituted C₃-C₆ cycloalkyl; and all other variables are as defined in any one of the first through hundred-and-fourth embodiments.

In a seventieth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

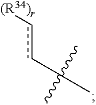

and all other variables are as defined in any one of the first through hundred-and-fourth embodiments.

In a seventy-first embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

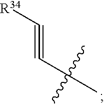

and all other variables are as defined in any one of the first through hundred-and-fourth embodiments.

In a seventy-second embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

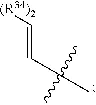

and all other variables are as defined in any one of the first through hundred-and-fourth embodiments.

In a seventy-third embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R³⁴ is hydrogen, a substituted or an unsubstituted C₁-C₆ alkyl, or C₁-C₆ alkoxy, wherein:
when a C₁-C₆ alkyl represented by R³⁴ is substituted, the C₁-C₆ alkyl is substituted with one or more groups each independently selected from halogen, —CN, —OH, and C₁-C₄ alkoxy;
and all other variables are as defined in any one of the first through hundred-and-fourth embodiments.

In a seventy-fourth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, R³⁴ is hydrogen, a substituted or an unsubstituted C₁-C₄ alkyl, or C₁-C₄ alkoxy, wherein:
when a C₁-C₄ alkyl represented by R³⁴ is substituted, the C₁-C₄ alkyl is substituted with one or more groups each independently selected from F, Cl, —CN, —OH, and C₁-C₂ alkoxy; and all other variables are as defined in any one of the first through hundred-and-fourth embodiments. In some embodiments of the seventy-fourth embodiment, R³⁴ is hydrogen.

In a seventy-fifth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is halogen, and all other variables are as defined in any one of the first through hundred-and-fourth embodiments.

In a seventy-sixth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is F, and all other variables are as defined in any one of the first through hundred-and-fourth embodiments.

In a seventy-seventh embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is Cl, and all other variables are as defined in any one of the first through hundred-and-fourth embodiments.

In a seventy-eighth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, with the exception of Formula (I) where X¹ is —CR^{B1}; X² is —CR^{B2}; Y¹ is —CR^{C2}; and Y² is —CR^{C3}, and Formula (Ib) where Y¹ is —CR^{C2}; Y² is —CR^{C3}, and Z is halogen, and all other variables are as defined in any one of the first through hundred-and-fourth embodiments.

In a seventy-ninth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, with the exception of Formula (I) where X¹ is —CR^{B1}; X² is —CR^{B2}; Y¹ is —CR^{C2}; and Y² is —CR^{C3}, and Formula (Ib) where Y¹ is —CR^{C2}; Y² is —CR^{C3}, and Z is F, and all other variables are as defined in any one of the first through hundred-and-fourth embodiments.

In an eightieth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, with the exception of Formula (I) where X¹ is —CR^{B1}; X² is —CR^{B2}; Y¹ is —CR^{C2}; and Y² is —CR^{C3}, and Formula (Ib) where Y¹ is —CR^{C2}; Y² is —CR^{C3}, and Z is Cl, and all other variables are as defined in any one of the first through hundred-and-fourth embodiments.

In an eighty-first embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is —CN, and all other variables are as defined in any one of the first through hundred-and-fourth embodiments.

In an eighty-second embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted C₁-C₆ alkyl; and all other variables are as defined in any one of the first through hundred-and-fourth embodiments.

In an eighty-third embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted C₁-C₆ alkyl, wherein:
when a C₁-C₆ alkyl represented by Z is substituted, the C₁-C₆ alkyl is substituted with one or more groups each independently selected from halogen, —CN, —OH, C₁-C₄ alkoxy, a C₃-C₆ cycloalkyl, and a 4- to 8-membered heterocyclyl;
and all other variables are as defined in any one of the first through hundred-and-fourth embodiments.

In an eighty-fourth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted C₁-C₄ alkyl, wherein:
when a C₁-C₄ alkyl represented by Z is substituted, the C₁-C₄ alkyl is substituted with one or more groups each independently selected from F, Cl, —CN, —OH, C₁-C₂ alkoxy, a C₃-C₅ cycloalkyl, and a 5- or 6-membered heterocyclyl;
and all other variables are as defined in any one of the first through hundred-and-fourth embodiments. In some embodiments of the eighty-fourth embodiment, Z is an unsubstituted C₁-C₄ alkyl (e.g., methyl, ethyl, n-propyl, iso-propyl, n-butyl, iso-butyl, sec-butyl and tert-butyl). In some embodiments of the eighty-fourth embodiment, Z is a substituted C₁-C₄ alkyl substituted with one or more (e.g., 1, 2, 3, 4 or 5) F. For example, Z is —CH₂F, —CHF₂, —CF₃, —CH₂CH₂F, —CH₂CHF₂, —CH₂CF₃.

In an eighty-fifth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted C₂-C₆ alkenyl (e.g., a C₂-C₆ alkenyl optionally substituted with one or more groups selected from halogen, —CN, —OH, and C₁-C₂ alkoxy, etc.); and all other variables are as defined in any one of the first through hundred-and-fourth embodiments.

In an eighty-sixth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted C₂-C₆ alkenyl (e.g., C₂-C₆ alkenyl, C₂-C₄ alkenyl), wherein:
when a C₂-C₆ alkenyl (e.g., C₂-C₆ alkenyl, C₂-C₄ alkenyl) represented by Z is substituted, the C₂-C₆ alkenyl (e.g., C₂-C₆ alkenyl, C₂-C₄ alkenyl) is substituted with one or more groups each independently selected from halogen, —CN, —OH, C₁-C₄ alkoxy, a C₃-C₆ cycloalkyl, and a 4- to 8-membered heterocyclyl;
and all other variables are as defined in any one of the first through hundred-and-fourth embodiments. In some embodiments of the eighty-sixth embodiment, Z is an unsubstituted C₂-C₆ alkenyl (e.g., C₂-C₆ alkenyl, C₂-C₄ alkenyl).

In an eighty-seventh embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted C₂-C₆ alkynyl (e.g., a C₂-C₆ alkynyl optionally substituted with one or more groups selected from halogen, —CN, —OH, and C₁-C₂ alkoxy, etc.); and all other variables are as defined in any one of the first through hundred-and-fourth embodiments. In some embodiments of the eighty-seventh embodiment, Z is an unsubstituted C₂-C₆ alkynyl.

In an eighty-eighth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted C₃-C₆ cycloalkyl; and all other variables are as defined in any one of the first through hundred-and-fourth embodiments.

In a eighty-ninth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted C₃-C₆ cycloalkyl, wherein:
when a C₃-C₆ cycloalkyl represented by Z is substituted, the C₃-C₆ cycloalkyl is substituted with one or more groups each independently selected from halogen, —CN, —OH, C₁-C₆ alkyl, C₁-C₆ alkyl substituted with one or more groups each independently selected from —OH, C₁-C₄ alkoxy, C₁-C₆ haloalkyl, and C₁-C₆ alkoxy;
and all other variables are as defined in any one of the first through hundred-and-fourth embodiments.

In a ninetieth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted C₃-C₅ cycloalkyl, wherein:
when a C₃-C₅ cycloalkyl represented by Z is substituted, the C₃-C₅ cycloalkyl is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₄haloalkyl, and C₁-C₄ alkoxy;
and all other variables are as defined in any one of the first through hundred-and-fourth embodiments. In some embodiments of the ninetieth embodiment, Z is a substituted or an unsubstituted monocyclic C₃-C₅ cycloalkyl (e.g., cyclopropyl, cyclobutyl and cyclopentyl). In some embodiments of the ninetieth embodiment, Z is an unsubstituted monocyclic C₃-C₅ cycloalkyl.

In a ninety-first embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted phenyl; and all other variables are as defined in any one of the first through hundred-and-fourth embodiments.

In a ninety-second embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted phenyl, wherein:
when a phenyl represented by Z is substituted, the phenyl is substituted with one or more groups each independently selected from halogen, —CN, —OH, C₁-C₆ alkyl, C₁-C₆ alkyl substituted with one or more groups each independently selected from —OH, C₁-C₄ alkoxy, C₁-C₆ haloalkyl, and C₁-C₆ alkoxy;
and all other variables are as defined in any one of the first through hundred-and-fourth embodiments.

In a ninety-third embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted phenyl, wherein:
when a phenyl represented by Z is substituted, the phenyl is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₆ haloalkyl, and C₁-C₄ alkoxy;
and all other variables are as defined in any one of the first through hundred-and-fourth embodiments. In some embodiments of the ninety-third embodiment, Z is a substituted phenyl such as a phenyl substituted 1, 2, 3, 4 or 5 times with halogen (e.g., F).

In a ninety-fourth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted 4- to 10-membered heterocyclyl; and all other variables are as defined in any one of the first through hundred-and-fourth embodiments.

In a ninety-fifth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted 4- to 10-membered heterocyclyl, wherein:
when a 4- to 10-membered heterocyclyl represented by Z is substituted, the 4- to 10-membered heterocyclyl is substituted with one or more groups each independently selected from halogen, —CN, —OH, C₁-C₆ alkyl, C₁-C₆ alkyl substituted with one or more groups each independently selected from —OH, C₁-C₄ alkoxy, C₁-C₆ haloalkyl, and C₁-C₆ alkoxy;
and all other variables are as defined in any one of the first through hundred-and-fourth embodiments.

In a ninety-sixth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted 4- to 10-membered heterocyclyl, or a substituted or an unsubstituted 4- to 8-membered heterocyclyl, or a substituted or an unsubstituted 5- to 6-membered heterocyclyl, or a substituted or an unsubstituted 5-membered heterocyclyl, or a substituted or an unsubstituted 6-membered heterocyclyl (e.g., a substituted or an unsubstituted 6-membered heterocyclyl containing a double bond and/or at least one oxygen atom), wherein:
when a 4- to 10-membered heterocyclyl, or a substituted or an unsubstituted 4- to 8-membered heterocyclyl, or a substituted or an unsubstituted 5- to 6-membered heterocyclyl, or a substituted or an unsubstituted 5-membered heterocyclyl, or a substituted or an unsubstituted 6-membered heterocyclyl (e.g., a substituted or an unsubstituted 6-membered heterocyclyl containing a double bond and/or at least one oxygen atom) represented by Z is substituted, the 4- to 10-membered heterocyclyl, or the substituted or an unsubstituted 4- to 8-membered heterocyclyl, or the substituted or an unsubstituted 5- to 6-membered heterocyclyl, or the substituted or an unsubstituted 5-membered heterocyclyl, or the substituted or an unsubstituted 6-membered heterocyclyl (e.g., a substituted or an unsubstituted 6-membered heterocyclyl containing a double bond and/or at least one oxygen atom) is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₄haloalkyl (e.g., —CH₂F, —CHF₂, —CF₃, —CH₂CH₂F, —CH₂CHF₂, —CH₂CF₃), and C₁-C₄ alkoxy;
and all other variables are as defined in any one of the first through hundred-and-fourth embodiments. For example, Z can be substituted azetidinyl, unsubstituted azetidinyl, substituted tetrahydrofuranyl, unsubstituted tetrahydrofuranyl, substituted pyrrolidinyl and unsubstituted pyrrolidinyl, substituted piperidinyl, unsubstituted piperidinyl, substituted morpholinyl, unsubstituted morpholinyl, substituted 3,6-dihydro-2H-pyranyl, unsubstituted 3,6-dihydro-2H-pyranyl, substituted 2,5-dihydrofuranyl, unsubstituted 2,5-dihydrofuranyl, substituted 3-oxabicyclo[3.1.0]hexane, unsubstituted 3-oxabicyclo[3.1.0]hexane, substituted 1-oxa-6-azaspiro[3.4]octane or unsubstituted 1-oxa-6-azaspiro[3.4]octane.

In a ninety-seventh embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted 4- to 10-membered heterocyclyl, wherein:
when a 4- to 10-membered heterocyclyl represented by Z is substituted, the 4- to 10-membered heterocyclyl is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₄ haloalkyl (e.g., —CH₂F, —CHF₂, —CF₃, —CH₂CH₂F, —CH₂CHF₂, —CH₂CF₃), and C₁-C₄ alkoxy;
and all other variables are as defined in any one of the first through hundred-and-fourth embodiments.

In a ninety-eighth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted 5- to 10-membered heteroaryl; and all other variables are as defined in any one of the first through hundred-and-fourth embodiments. For example, Z can be substituted or unsubstituted pyridinyl, substituted or unsubstituted pyrazolyl, substituted or unsubstituted thiazolyl, substituted or unsubstituted isothiazolyl, substituted or unsubstituted imidazolyl, etc.).

In a ninety-ninth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted 5- to 10-membered heteroaryl, wherein:
when a 5- to 10-membered heteroaryl represented by Z is substituted, the 5- to 10-membered heteroaryl is substituted with one or more groups each independently selected from halogen (e.g., F, Cl), —CN, —OH, C₁-C₆ alkyl, C₁-C₆ alkyl substituted with one or more groups each independently selected from —OH, C₁-C₄ alkoxy, C₁-C₆ haloalkyl (e.g., —CH₂F, —CHF₂, —CF₃, —CH₂CH₂F, —CH₂CHF₂, —CH₂CF₃), and C₁-C₆ alkoxy;
and all other variables are as defined in any one of the first through hundred-and-fourth embodiments.

In a hundredth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is a substituted or an unsubstituted 5- to 10-membered heteroaryl, wherein:
when a 5- to 10-membered heteroaryl represented by Z is substituted, the 5- to 10-membered heteroaryl is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₄ haloalkyl (e.g., —CH₂F, —CHF₂, —CF₃, —CH₂CH₂F, —CH₂CHF₂, —CH₂CF₃), and C₁-C₄ alkoxy;
and all other variables are as defined in any one of the first through hundred-and-fourth embodiments. In some embodiments of the hundredth embodiment, Z is a substituted or an unsubstituted 5- to 10-membered heteroaryl (e.g., a substituted or an unsubstituted 5- to 10-membered heteroaryl, a substituted or an unsubstituted 5- to 8-membered heteroaryl, a substituted or an unsubstituted 5- to 6-membered heteroaryl and a substituted or an unsubstituted 5- to 6-membered monocyclic heteroaryl such as a substituted or an unsubstituted pyrrolyl or a substituted or an unsubstituted pyridinyl), wherein the substituted heteroaryl is substituted with C₁-C₄ alkyl (e.g., methyl).

In a hundred-and-first embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), (IIId-1), (IVa), (IVa-1), (Va), or (Va-1), or a pharmaceutically acceptable salt thereof, and unless specifically defined differently:
any C₁-C₆ alkyl includes C₁-C₆ alkyl, C₁-C₄ alkyl, C₁-C₃ alkyl, C₁-C₂ alkyl, methyl, ethyl, etc.;
any C₁-C₄ alkyl includes C₁-C₄ alkyl, C₁-C₃ alkyl, C₁-C₂ alkyl, methyl, ethyl, etc.; any C₁-C₃ alkyl includes C₁-C₃ alkyl, C₁-C₂ alkyl, methyl, ethyl, etc.;
any C₁-C₂ alkyl includes C₁-C₂ alkyl, methyl, ethyl, etc.;
any C₂-C₆ alkenyl includes C₂-C₆ alkenyl, C₂-C₄ alkenyl, C₂-C₃ alkenyl, etc.; any C₂-C₄ alkenyl includes C₂-C₄ alkenyl, C₂-C₃ alkenyl, etc.;
any C₂-C₆ alkynyl includes C₂-C₆ alkynyl, C₂-C₄ alkynyl, C₂-C₃ alkynyl, etc.; any C₂-C₄ alkynyl includes C₂-C₄ alkynyl, C₂-C₃ alkynyl, etc.;
any C₁-C₆ alkoxy includes C₁-C₆ alkoxy, C₁-C₄ alkoxy, C₁-C₃ alkoxy, C₁-C₂ alkoxy, methoxy, ethoxy, etc.;
any C₁-C₄ alkoxy includes C₁-C₄ alkoxy, C₁-C₃ alkoxy, C₁-C₂ alkoxy, methoxy, ethoxy, etc.;
any C₁-C₃ alkoxy includes C₁-C₃ alkoxy, C₁-C₂ alkoxy, methoxy, ethoxy, etc.; any C₁-C₂ alkoxy includes C₁-C₂ alkoxy, methoxy, ethoxy, etc.;
any C₁-C₆ heteroalkyl (e.g., —(CH₂)ₘO(CH₂)ₙCH₃, —(CH₂)ₘNH(CH₂)ₙCH₃, —(CH₂)ₘS(CH₂)ₙCH₃, where m and n are each an integer) includes C₁-C₆ heteroalkyl, C₁-C₄ heteroalkyl, C₁-C₃ heteroalkyl, C₁-C₂ heteroalkyl, etc.;
any C₁-C₄ heteroalkyl includes C₁-C₄ heteroalkyl, C₁-C₃ heteroalkyl, C₁-C₂ heteroalkyl, etc.;
any C₁-C₃ heteroalkyl includes C₁-C₃ heteroalkyl, C₁-C₂ heteroalkyl, etc.; any C₃-C₆ cycloalkyl includes C₃-C₆ cycloalkyl, C₄-C₆ cycloalkyl, C₃-C₅ cycloalkyl, C₃-C₄ cycloalkyl, cyclopropyl, cyclobutyl, cyclopentyl, cyclohexyl, etc.;
any C₄-C₆ cycloalkyl includes C₄-C₆ cycloalkyl, cyclobutyl, cyclopentyl, cyclohexyl, etc.;
any C₃-C₅ cycloalkyl includes C₃-C₅ cycloalkyl, C₃-C₄ cycloalkyl, cyclopropyl, cyclobutyl, cyclopentyl, etc.;
any C₃-C₄ cycloalkyl includes cyclopropyl or cyclobutyl;
any 6- or 10-membered aryl includes 6- or 10-membered aryl, phenyl, etc.;
any 4- to 10-membered heterocyclyl includes 4- to 10-membered heterocyclyl, 4- to 9-membered heterocyclyl, 4- to 8-membered heterocyclyl, 5- to 10-membered heterocyclyl, 5- to 9-membered heterocyclyl, 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 4- to 9-membered heterocyclyl includes 4- to 9-membered heterocyclyl, 4- to 8-membered heterocyclyl, 5- to 9-membered heterocyclyl, 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 5- to 10-membered heterocyclyl includes 5- to 10-membered heterocyclyl, 4- to 9-membered heterocyclyl, 4- to 8-membered heterocyclyl, 5- to 9-membered heterocyclyl, 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 5- to 9-membered heterocyclyl includes 5- to 9-membered heterocyclyl, 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 5- to 8-membered heterocyclyl includes 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 7- to 9-membered heterocyclyl includes 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 7- or 8-membered heterocyclyl includes 7- or 8-membered heterocyclyl, 7- or 8-membered spiro heterocyclyl, 7-membered spiro heterocyclyl, 8-membered spiro heterocyclyl, etc., each of which containing, for example, 1, 2, 3, or 4 nitrogen atoms;
any 5- to 10-membered heteroaryl includes 5- to 10-membered heteroaryl, 5- to 8-membered heteroaryl, 5- or 6-membered heteroaryl, 5-membered heteroaryl, 6-heteroaryl, etc., each of which containing, for example, 1, 2, 3, or 4 nitrogen atoms;
any 5- to 8-membered heteroaryl includes 5- to 8-membered heteroaryl, 5- or 6-membered heteroaryl, 5-membered heteroaryl, 6-heteroaryl, etc., each of which containing, for example, 1, 2, 3, or 4 nitrogen atoms; and
any 5- or 6-membered heteroaryl includes 5- or 6-membered heteroaryl, 5-membered heteroaryl, 6-heteroaryl, each of which containing, for example, 1, 2, 3, or 4 nitrogen atoms; and all other variables are as defined in any one of the first through hundred-and-fourth embodiments.

In a hundred-and-second embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), (IIId-1), (IVa), (IVa-1), (Va), or (Va-1), or a pharmaceutically acceptable salt thereof, and unless specifically defined differently, any group including any of the groups described in the hundred-and-second embodiment that is substituted can be substituted with one or more groups selected from:
halogen (including e.g., F, Cl, Br, etc.);
—CN;
—OH;
C₁-C₆ alkyl (including e.g., C₁-C₆ alkyl, C₁-C₄ alkyl, C₁-C₂ alkyl, methyl, ethyl, etc.);
C₁-C₆ heteroalkyl (including e.g., C₁-C₆ heteroalkyl, C₁-C₄ heteroalkyl, C₁-C₂ alkyl, etc.);
C₁-C₆ haloalkyl (including e.g., C₁-C₆ haloalkyl, C₁-C₄ haloalkyl, C₁-C₂ haloalkyl, halomethyl, haloethyl, etc.);
C₁-C₆ alkyl (including e.g., C₁-C₆ alkyl, C₁-C₄ alkyl, C₁-C₂ alkyl, methyl, ethyl, etc.) substituted with C₁-C₆ alkoxy (including e.g., C₁-C₆ alkoxy, C₁-C₄ alkoxy, C₁-C₂ alkoxy, methoxy, ethoxy, etc.);
C₁-C₆ alkoxy (including e.g., C₁-C₆ alkoxy, C₁-C₄ alkoxy, C₁-C₂ alkoxy, methoxy, ethoxy, etc.);
C₁-C₆ haloalkoxy (including e.g., C₁-C₆ haloalkoxy, C₁-C₄ haloalkoxy, C₁-C₂ haloalkoxy, halomethoxy, haloethoxy, etc.); and
C₃-C₆ cycloalkyl (including e.g., C₃-C₆ cycloalkyl, C₄-C₆ cycloalkyl, C₃-C₅ cycloalkyl, C₃-C₄ cycloalkyl, cyclopropyl, cyclobutyl, cyclopentyl, cyclohexyl, etc.);
and all other variables are as defined in any one of the first through hundred-and-fourth embodiments.

In a hundred-and-third embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), (IIId-1), (IVa), (IVa-1), (Va), or (Va-1), or a pharmaceutically acceptable salt thereof, and unless specifically defined differently, any group including any of the groups described in the hundred-and-second embodiment that is substituted can be substituted with one or more groups selected from:
halogen (including e.g., F, Cl, Br, etc.);
—CN;
—OH;
C₁-C₆ alkyl (including e.g., C₁-C₆ alkyl, C₁-C₄ alkyl, C₁-C₂ alkyl, methyl, ethyl, etc.);
C₁-C₆ haloalkyl (including e.g., C₁-C₆ haloalkyl, C₁-C₄ haloalkyl, C₁-C₂ haloalkyl, halomethyl, haloethyl, etc.);
C₁-C₆ alkyl (including e.g., C₁-C₆ alkyl, C₁-C₄ alkyl, C₁-C₂ alkyl, methyl, ethyl, etc.) substituted with C₁-C₆ alkoxy (including e.g., C₁-C₆ alkoxy, C₁-C₄ alkoxy, C₁-C₂ alkoxy, methoxy, ethoxy, etc.); and
C₁-C₆ alkoxy (including e.g., C₁-C₆ alkoxy, C₁-C₄ alkoxy, C₁-C₂ alkoxy, methoxy, ethoxy, etc.);
and all other variables are as defined in any one of the first through hundred-and-fourth embodiments.

In a hundredth-and-fourth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), (IIId-1), (IVa), (IVa-1), (Va), or (Va-1), or a pharmaceutically acceptable salt thereof, and unless specifically defined differently, any group including any of the groups described in the hundred-and-second embodiment that is substituted can be substituted with one or more groups selected from:
halogen (including e.g., F, Cl, Br, etc.);
—CN;
—OH;
C₁-C₆ alkyl (including e.g., C₁-C₆ alkyl, C₁-C₄ alkyl, C₁-C₂ alkyl, methyl, ethyl, etc.);
C₁-C₆ haloalkyl (including e.g., C₁-C₆ haloalkyl, C₁-C₄ haloalkyl, C₁-C₂ haloalkyl, halomethyl, haloethyl, etc.); and
C₁-C₆ alkoxy (including e.g., C₁-C₆ alkoxy, C₁-C₄ alkoxy, C₁-C₂ alkoxy, methoxy, ethoxy, etc.)
and all other variables are as defined in any one of the first through hundred-and-fourth embodiments.

Embodiments whereby one or more hydrogen atoms of a compound represented by Formula (I), (Ia), (Ib), (Ic), (Id), (II), (II-1), (IIIa), (IIIb), (IIIc), (IIId), (IIIa-1), (IIIb-1), (IIIc-1), (IIId-1), (IVa), (IVa-1), (Va), or (Va-1), or a pharmaceutically acceptable salt thereof, are replaced by or substituted with deuterium, are contemplated, unless specifically defined differently, including embodiments whereby one or more hydrogen atoms or all hydrogen atoms in any substituted alkyl group (any substituted C₁-C₆ alkyl, substituted C₁-C₄ alkyl, substituted C₁-C₃ alkyl, substituted C₁-C₂ alkyl, substituted methyl, substituted ethyl, etc.) in a compound represented by Formula (I), (Ia), (Ib), (Ic), (Id), (II), (II-1), (IIIa), (IIb), (IIIc), (IId), (IIIa-1), (IIIb-1), (IIIc-1), (IIId-1), (IVa), (IVa-1), (Va), or (Va-1), or a pharmaceutically acceptable salt thereof, are replaced by or substituted with deuterium. As an example, a compound of Formula (I) or (I′) as defined herein, or a pharmaceutically acceptable salt of any of the foregoing, may be deuterated to form a compound represented by the structural formula:

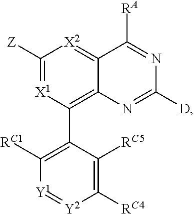

or a pharmaceutically acceptable thereof. In these instances, these deuterated analogs may be prepared using the procedures described in at least General Schemes 1 and 2 provided herein, by replacing the reagent HC(OEt)₃ or HC(OMe)₃ in the third reaction step in either one of General Schemes 1 and 2 (i.e., the reaction step between amidation and N-protection), with DC(OEt)₃ or DC(OMe)₃, respectively.

In a hundredth-and-fifth embodiment, this disclosure relates to a compound represented by the following Formula (I′):

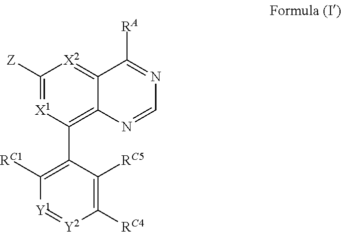

or a pharmaceutically acceptable salt thereof, wherein:
X¹ is —CR^{B1} or N (nitrogen);
X² is —CR^{B2} or N (nitrogen);
Y¹ is —CR^{C2} or N (nitrogen);
Y² is —CR^{C3} or N (nitrogen), provided that Y¹ and Y² are not both N (nitrogen);
Z is

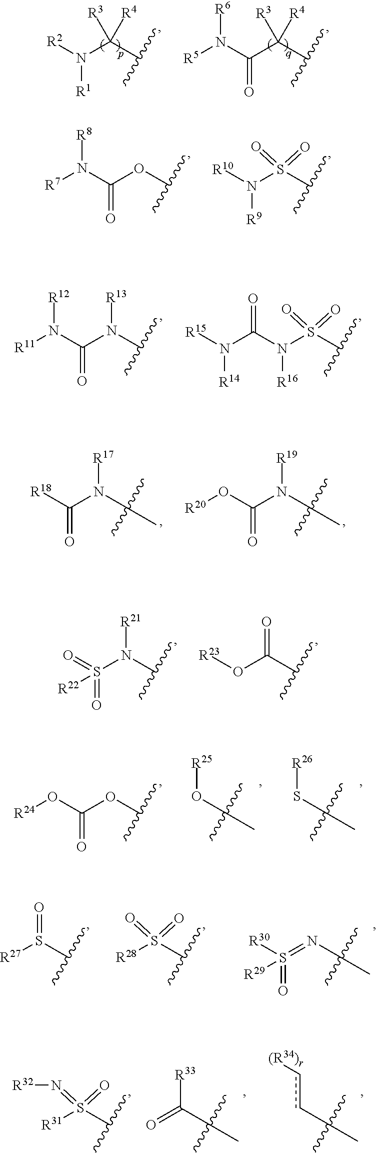

halogen, —CN, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl, wherein:
   [symbol] is a double bond or a triple bond;
   p and q are each independently 0 or 1;
   r is 1 or 2;
   R¹, R², R⁵, R⁶, R⁷, R⁸, R⁹, R¹⁰, R¹¹, R¹², R¹⁴, and R¹⁵ are each independently hydrogen, a substituted or an unsubstituted C₁-C₆ alkyl, a substituted or an unsubstituted C₁-C₆ heteroalkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 4- to 10-membered heterocyclyl, or a substituted or an unsubstituted 5- to 10-membered heteroaryl; or alternatively
   R¹ and R², R⁵ and R⁶, R⁷ and R⁸, R⁹ and R¹⁰, R¹¹ and R¹², or R¹⁴ and R¹⁵, together with the respective nitrogen atom connected thereto, each independently form a substituted or an unsubstituted 4- to 10-membered heterocyclyl, or a substituted or an unsubstituted 5- to 10-membered heteroaryl;
   R³ and R⁴ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; or alternatively
   R³ and R⁴, together with the carbon atom connected thereto, each independently form a substituted or an unsubstituted C₃-C₆ cycloalkyl or a substituted or an unsubstituted 4- to 6-membered heterocyclyl;
   R¹³, R¹⁶, R¹⁷, R¹⁹, and R²¹ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, or a substituted or an unsubstituted C₃-C₆ cycloalkyl;
   R¹⁸, R²⁰, R²³, R²⁴, R²⁵, and R²⁶ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl;
   R²², R²⁷, R²⁸, R²⁹, R³⁰, and R³¹ are each independently a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl;
   R³² is hydrogen, —CN, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl;
   R³³ and R³⁴ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; and
R^{A} is —NR³⁵R³⁶, wherein:
   R³⁵ and R³⁶ are each independently hydrogen, a substituted or an unsubstituted C₁-C₆ alkyl, a substituted or an unsubstituted C₃-C₆ cycloalkyl, or a substituted or an unsubstituted C₄-C₆ heterocyclyl; or alternatively
   one of R³⁵ and R³⁶ is hydrogen or a substituted or an unsubstituted C₁-C₆ alkyl, and the other is a substituted or an unsubstituted 6- or 10-membered aryl or a substituted or an unsubstituted 5- to 10-membered heteroaryl;
R^{B1} and R^{B2} are each independently hydrogen, halogen, —CN, —OH, —OR³⁷, —SR³⁸, a substituted or an unsubstituted C₁-C₆ alkyl, a substituted or an unsubstituted C₁-C₆ heteroalkyl, a substituted or an unsubstituted C₂-C₆ alkenyl, a substituted or an unsubstituted C₂-C₆ alkynyl, a substituted or an unsubstituted C₃-C₆ cycloalkyl, or a substituted or an unsubstituted 5- to 10-membered heteroaryl, wherein:
   R³⁷ and R³⁸, for each instance, are each independently a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, or a substituted or an unsubstituted C₄-C₆ heterocyclyl;
R^{C1}, R^{C2}, and R^{C3} are each independently hydrogen, halogen, —CN, —OH, —OR³⁹, —SR⁴⁰, a substituted or an unsubstituted C₁-C₆ alkyl, a substituted or an unsubstituted C₂-C₆ alkenyl, a substituted or an unsubstituted C₂-C₆ alkynyl, or a substituted or an unsubstituted C₃-C₆ cycloalkyl;
R^{C4} and R^{C5} are each independently hydrogen, halogen, —CN, —OH, —OD, —OR³⁹, —SR⁴⁰, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, or a substituted or an unsubstituted C₃-C₆ cycloalkyl; or alternatively
R^{C4} and R^{C5}, together with the carbon atom that each of R^{C4} and R^{C5} is respectively connected thereto, form a 5-membered a substituted or an unsubstituted heteroaryl, wherein:
   R³⁹ and R⁴⁰, for each instance, are each independently a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, or a substituted or an unsubstituted C₄-C₆ heterocyclyl;
provided that R^{C1}, R^{C2}, R^{C3}, R^{C4} and R^{C5} are not all hydrogen (for example, one of R^{C1}, R^{C2}, R^{C3}, R^{C4} and R^{C5} is not hydrogen, two of R^{C1}, R^{C2}, R^{C3}, R^{C4} and R^{C5}, three of R^{C1}, R^{C2}, R^{C3}, R^{C4} and R^{C5} are not hydrogen, four of R^{C1}, R^{C2}, R^{C3}, R^{C4} and R^{C5} or all of R^{A}, R^{B} and R^{C} are not hydrogen); and
with the proviso that the compound represented by Formula (I′) is not N-butyl-6-chloro-N-ethyl-8-(2,4,6-trimethylphenyl)-4-quinazolinamine.

In a hundredth-and-sixth embodiment, in a compound represented by Formula (I′), or a pharmaceutically acceptable salt thereof, Y¹ is —CR^{C2} and Y² is —CR^{C3} and all other variables are as defined in any one of the hundredth-and-fifth through two-hundred-and-tenth embodiments.

In a hundredth-and-seventh embodiment, in a compound represented by Formula (I′), or a pharmaceutically acceptable salt thereof, Y¹ is —CR^{C2} and Y² is N; and all other variables are as defined in any one of the hundredth-and-fifth through two-hundred-and-tenth embodiments.

In a hundredth-and-eighth embodiment, in a compound represented by Formula (I′), or a pharmaceutically acceptable salt thereof, Y¹ is N and Y² is —CR^{C3}; and all other variables are as defined in any one of the hundredth-and-fifth through two-hundred-and-tenth embodiments.

In a hundredth-and-nineth embodiment, in a compound represented by Formula (I), or a pharmaceutically acceptable salt thereof, the 5-membered heteroaryl formed by R^{C4} and R^{C5}, together with the carbon atom that each of R^{C4} and R^{C5} is respectively connected thereto, is a substituted or an unsubstituted 5-membered heteroaryl containing 1 or 2 nitrogen atoms; and all other variables are as defined in any one of the hundredth-and-fifth through two-hundred-and-tenth embodiments.

In a hundredth-and-tenth embodiment, a compound of this disclosure is represented by the following Formula (I′a), (I′b), (I′c), or (I′d):

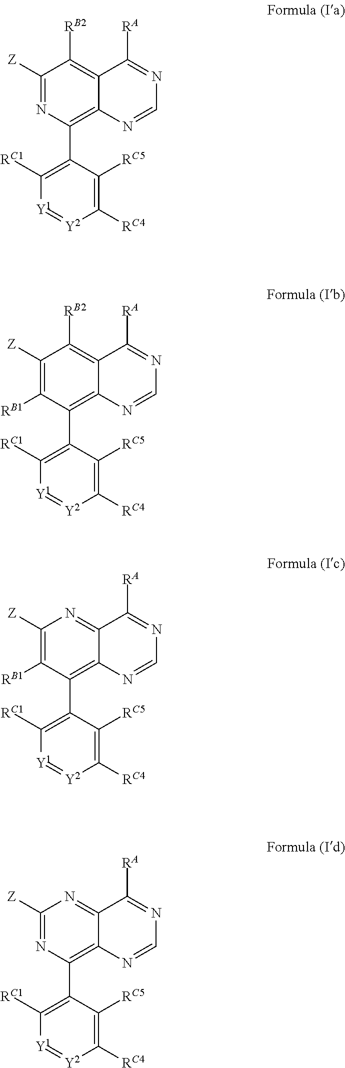

or a pharmaceutically acceptable salt thereof; and all other variables are as defined in any one of the hundredth-and-fifth through two-hundred-and-tenth embodiments.

In a hundredth-and-eleventh embodiment, in a compound represented by Formula (I′), (I′a), (I′b), (I′c), or (I′d), or a pharmaceutically acceptable salt thereof, the substituted or unsubstituted 5-membered heteroaryl formed by R^{C4} and R^{C5}, together with the carbon atom that each of R^{C4} and R^{C5} is respectively connected thereto, is a substituted or an unsubstituted 5-membered heteroaryl containing 1 nitrogen atom; and all other variables are as defined in any one of the hundredth-and-fifth through two-hundred-and-tenth embodiments.

In a hundredth-and-twelfth embodiment, in a compound represented by Formula (I′), (I′a), (I′b), (I′c), or (I′d), or a pharmaceutically acceptable salt thereof, the substituted or unsubstituted 5-membered heteroaryl formed by R^{C4} and R^{C5}, together with the carbon atom that each of R^{C4} and R^{C5} is respectively connected thereto, is a substituted or an unsubstituted 5-membered heteroaryl containing 2 nitrogen atoms; and all other variables are as defined in any one of the hundredth-and-fifth through two-hundred-and-tenth embodiments.

In a hundredth-and-thirteenth embodiment, in a compound represented by Formula (I′), (I′a), (I′b), (I′c), or (I′d), or a pharmaceutically acceptable salt thereof, R^{C1}, R^{C2}, R^{C3}, R^{C4} and R^{C5} are each independently hydrogen, halogen, —CN, —OH, —OR³⁹, —SR⁴⁰, a substituted or an unsubstituted C₁-C₆ alkyl, a substituted or an unsubstituted C₂-C₆ alkenyl, a substituted or an unsubstituted C₂-C₆ alkynyl, or a substituted or an unsubstituted C₃-C₆ cycloalkyl; and all other variables are as defined in any one of the hundredth-and-fifth through two-hundred-and-tenth embodiments.

In a hundredth-and-fourteenth embodiment, in a compound represented by Formula (I′), or a pharmaceutically acceptable salt thereof, where X is —CR^{B1}; X² is —CR^{B2}; Y¹ is —CR^{C2}; and Y² is —CR^{C3}, in a compound represented by Formula (I′b), or a pharmaceutically acceptable salt thereof, where Y¹ is —CR^{C2}; Y² is —CR^{C3}; and Z is

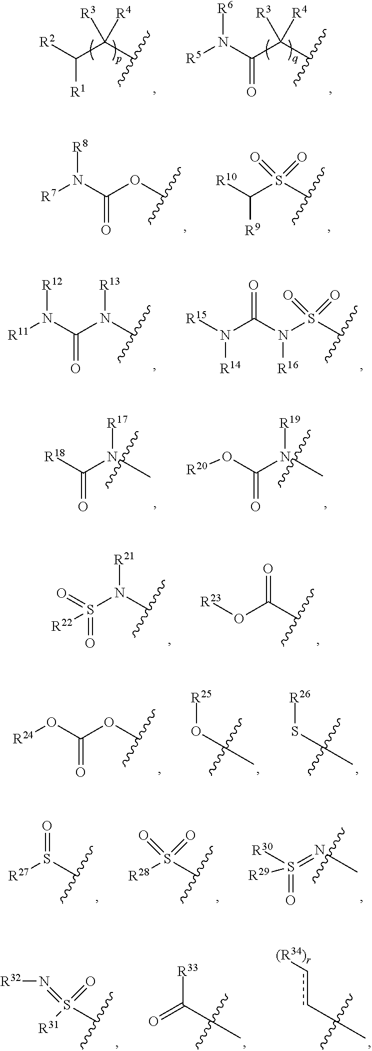

—CN, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; and all other variables are as defined in any one of the hundredth-and-fifth through two-hundred-and-tenth embodiments.

In a hundredth-and-fifteenth embodiment, a compound of this disclosure is represented by the following Formula (II′) or (II′-1):

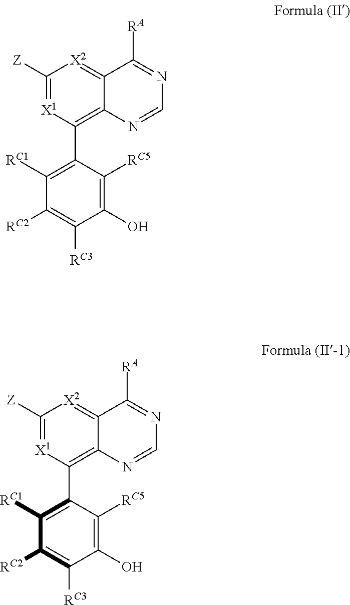

or a pharmaceutically acceptable salt thereof, and all other variables are as defined in any one of the hundredth-and-fifth through two-hundred-and-tenth embodiments.

In a hundredth-and-sixteenth embodiment, in a compound represented by Formula (I′), (I′a), (I′b), (I′c), (I′d), (II′), or (II′-1), or a pharmaceutically acceptable salt thereof, R³⁵ and R³⁶ are each independently hydrogen, a substituted or an unsubstituted C₁-C₆ alkyl, a substituted or an unsubstituted C₃-C₆ cycloalkyl, or a substituted or an unsubstituted C₄-C₆ heterocyclyl; and all other variables are as defined any one of the hundredth-and-fifth through two-hundred-and-tenth embodiments.

In a hundredth-and-seventeenth embodiment, in a compound represented by Formula (I′), (I′a), (I′b), (I′c), (I′d), (II′), or (II′-1), or a pharmaceutically acceptable salt thereof, R³⁵ and R³⁶ are each independently hydrogen, a substituted or an unsubstituted C₁-C₆ alkyl, or a substituted or an unsubstituted C₃-C₆ cycloalkyl; and all other variables are as defined any one of the hundredth-and-fifth through two-hundred-and-tenth embodiments.

In a hundredth-and-eighteenth embodiment, in a compound represented by Formula (I′), (I′a), (I′b), (I′c), (I′d), (II′), or (II′-1), or a pharmaceutically acceptable salt thereof, R³⁵ and R³⁶ are each independently hydrogen or a substituted or an unsubstituted C₁-C₆ alkyl; or R³⁵ and R³⁶ are each independently hydrogen or a substituted or an unsubstituted C₁-C₄ alkyl; or R³⁵ and R³⁶ are each independently hydrogen or a substituted or an unsubstituted C₁-C₃ alkyl; or R³⁵ and R³⁶ are each independently hydrogen or a substituted or an unsubstituted C₁-C₂ alkyl; and all other variables are as defined any one of the hundredth-and-fifth through two-hundred-and-tenth embodiments.

In a hundredth-and-nineteenth embodiment, a compound of this disclosure is represented by the following Formula (III′a), (III′b), (III′c), (III′d), (III′a-1), (III′b-1), (III′c-1), or (III′d-1):

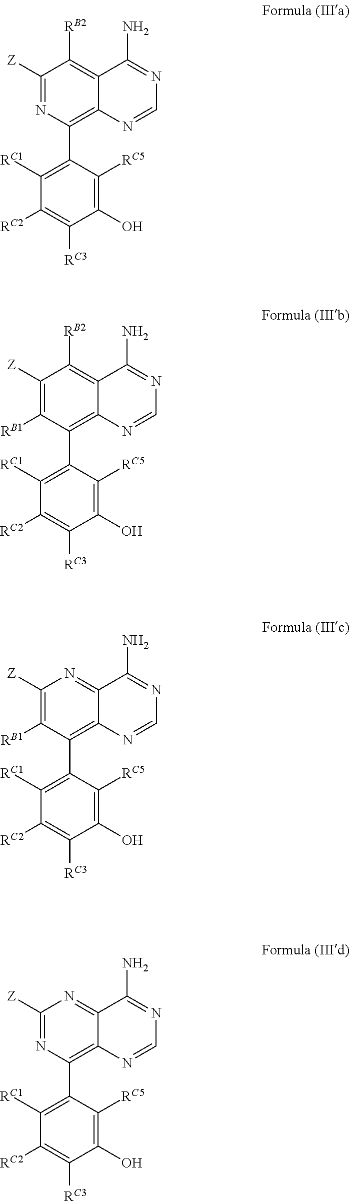

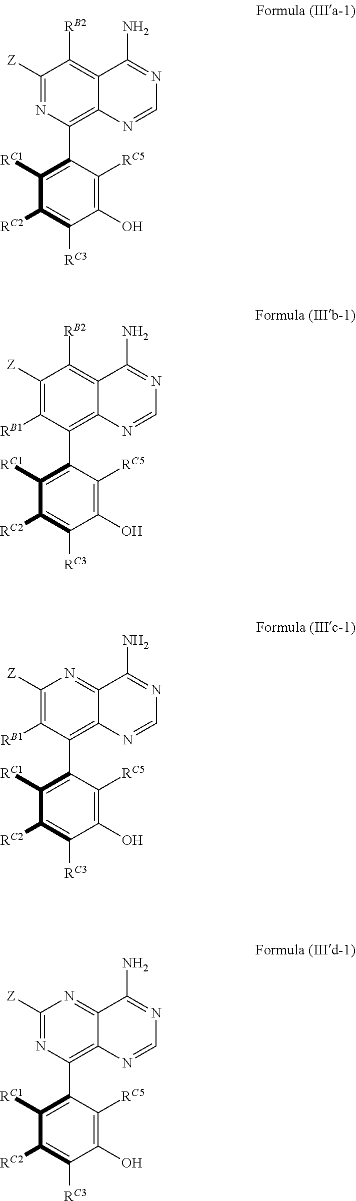

or a pharmaceutically acceptable salt thereof, and all other variables are as defined in any one of the hundredth-and-fifth through two-hundred-and-tenth embodiments.

In a hundredth-and-twentieth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1) or a pharmaceutically acceptable salt thereof, R^{C1}, R^{C2}, R^{C3} and R^{C5} are each independently hydrogen, halogen, —CN, —OH, —OR³⁹, a substituted or an unsubstituted C₁-C₆ alkyl, or a substituted or an unsubstituted C₃-C₆ cycloalkyl; wherein R³⁹ is a substituted or an unsubstituted C₁-C₆ alkyl or a substituted or an unsubstituted C₃-C₆ cycloalkyl; and all other variables are as defined in any one of the hundredth-and-fifth through two-hundred-and-tenth embodiments.

In a hundredth-and-twenty-first embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R^{C1}, R^{C2}, R^{C3} and R^{C5} are each independently hydrogen, halogen, —CN, —OH, —OR³⁹, a substituted or an unsubstituted C₁-C₆ alkyl or a substituted or an unsubstituted C₃-C₆ cycloalkyl; wherein R³⁹ is a substituted or an unsubstituted C₁-C₆ alkyl (e.g., a substituted or an unsubstituted C₁-C₆ alkyl, a substituted or an unsubstituted C₁-C₄ alkyl, a substituted or an unsubstituted C₁-C₃ alkyl, or a substituted or an unsubstituted C₁-C₂ alkyl, etc.); and all other variables are as defined in any one of the hundredth-and-fifth through two-hundred-and-tenth embodiments.

In a hundredth-and-twenty-second embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R^{C1}, R^{C2}, R^{C3} and R^{C5} are each independently hydrogen, F, Cl, Br, —CN, or a substituted or an unsubstituted C₁-C₆ alkyl; and all other variables are as defined in any one of the hundredth-and-fifth through two-hundred-and-tenth embodiments.

In a hundredth-and-twenty-third embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R^{C1}, R^{C2}, R^{C3} and R^{C5} are each independently hydrogen, Cl, methyl, —CD₃, —CF₃, or —CHF₂; and all other variables are as defined in any one of the hundredth-and-fifth through two-hundred-and-tenth embodiments.

In a hundredth-and-twenty-fourth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), (III′d-1), (IV′a), (IV′a-1), (V′a), or (V′a-1), or a pharmaceutically acceptable salt thereof, at least one of R^{C1}, R^{C2}, R^{C3} and R^{C5} is not hydrogen; and all other variables are as defined in any one of the hundredth-and-fifth through two-hundred-and-tenth embodiments.

In a hundredth-and-twenty-fifth embodiment, in a compound represented by Formula (I′), (I′a), (I′b), (I′c), (I′d), (II′), (II′-1) or a pharmaceutically acceptable salt thereof, at least two of R^{C1}, R^{C2}, R^{C3} and R^{C5} are not hydrogen; and all other variables are as defined in any one of the hundredth-and-fifth through two-hundred-and-tenth embodiments.

In a hundredth-and-twenty-sixth embodiment, in a compound represented by Formula (I′), (I′a), (I′b), (I′c), (I′d), (II′), or (II′-1), or a pharmaceutically acceptable salt thereof, R^{B1} and R^{B2} are each independently hydrogen, halogen, —CN, —OH, —OR³⁷, a substituted or an unsubstituted C₁-C₆ alkyl, a substituted or an unsubstituted C₁-C₆ heteroalkyl, or a substituted or an unsubstituted C₃-C₆ cycloalkyl; wherein R³⁷ is a substituted or an unsubstituted C₁-C₆ alkyl or a substituted or an unsubstituted C₃-C₆ cycloalkyl; and all other variables are as defined in any one of the hundredth-and-fifth through two-hundred-and-tenth embodiments.

In a hundredth-and-twenty-seventh embodiment, in a compound represented by Formula (I′), (I′a), (I′b), (I′c), (I′d), (II′), or (II′-1), or a pharmaceutically acceptable salt thereof, R^{B1} and R^{B2} are each independently hydrogen, F, Cl, Br, —CN, —OR³⁷, or a substituted or an unsubstituted C₁-C₆ alkyl; wherein R³⁷ is a substituted or an unsubstituted C₁-C₆ alkyl; and all other variables are as defined in any one of the hundredth-and-fifth through two-hundred-and-tenth embodiments.

In a hundredth-and-twenty-eighth embodiment, in a compound represented by Formula (I′), (I′a), (I′b), (I′c), (I′d), (II′), or (II′-1), or a pharmaceutically acceptable salt thereof, R^{B1} is each independently hydrogen, F, Cl, —CN, —OR³⁷, or a substituted or an unsubstituted C₁-C₆ alkyl; wherein R³⁷ is a substituted or an unsubstituted C₁-C₄ alkyl; and all other variables are as defined in any one of the hundredth-and-fifth through two-hundred-and-tenth embodiments.

In a hundredth-and-twenty-nineth embodiment, in a compound represented by Formula (I′), (I′a), (I′b), (I′c), (I′d), (II′), or (II′-1), or a pharmaceutically acceptable salt thereof, R^{B2} is hydrogen, F, methyl, or methoxy; and all other variables are as defined in any one of the hundredth-and-fifth through two-hundred-and-tenth embodiments.

In a hundredth-and-thirty embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1) or a pharmaceutically acceptable salt thereof, Z is

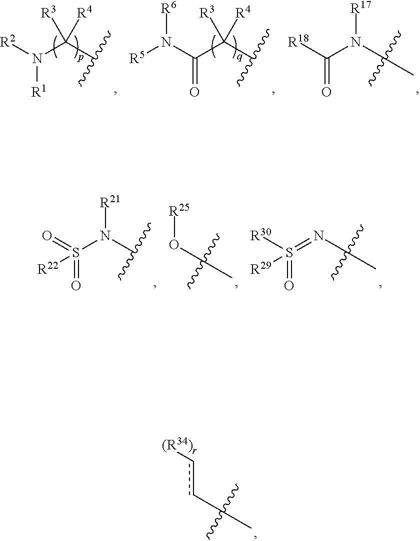

—CN, a substituted or an unsubstituted C₁-C₆ alkyl, a substituted or an unsubstituted C₂-C₆ alkenyl, a substituted or an unsubstituted C₃-C₆ cycloalkyl, a substituted or an unsubstituted phenyl, a substituted or an unsubstituted 4- to 10-membered heterocyclyl, or a substituted or an unsubstituted 5- to 10-membered heteroaryl; and all other variables are as defined in any one of the hundredth-and-fifth through two-hundred-and-tenth embodiments.

In a hundredth-and-thirty-first embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1) or a pharmaceutically acceptable salt thereof, Z is

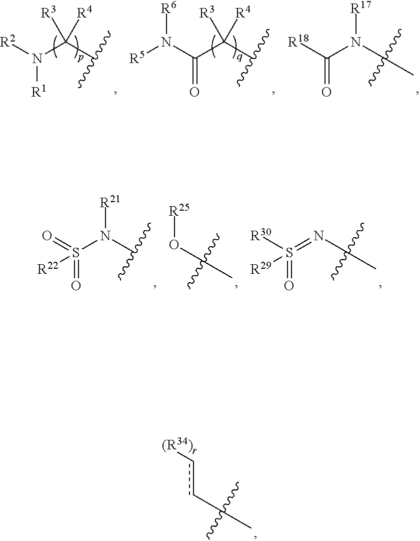

substituted or an unsubstituted C₁-C₆ alkyl, a substituted or an unsubstituted C₃-C₆ cycloalkyl, a substituted or an unsubstituted phenyl, or a substituted or an unsubstituted 5- to 10-membered heteroaryl; and all other variables are as defined in any one of the hundredth-and-fifth through two-hundred-and-tenth embodiments.

In a hundredth-and-thirty-second embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

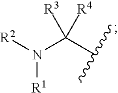

and all other variables are as defined in any one of the hundredth-and-fifth through two-hundred-and-tenth embodiments.

In a hundredth-and-thirty-third embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (IIId-1), or a pharmaceutically acceptable salt thereof, R³ and R⁴ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; and all other variables are as defined in any one of the hundredth-and-fifth through two-hundred-and-tenth embodiments.

In a hundredth-and-thirty-fourth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R³ and R⁴ are each independently hydrogen, a substituted or an unsubstituted C₁-C₆ alkyl, or a substituted or an unsubstituted C₃-C₆ cycloalkyl; and all other variables are as defined in any one of the hundredth-and-fifth through two-hundred-and-tenth embodiments.

In a hundredth-and-thirty-fifth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R³ and R⁴ are each independently hydrogen or a substituted or an unsubstituted C₁-C₄ alkyl; and all other variables are as defined in any one of the hundredth-and-fifth through two-hundred-and-tenth embodiments.

In a hundredth-and-thirty-sixth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R, R², R⁵, R⁶, R⁷, R⁸, R⁹, R¹⁰, R¹¹, R¹², R¹⁴, and R¹⁵ are each independently hydrogen, a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁-C₂ alkyl, —CD₃, etc.), a substituted or an unsubstituted C₁-C₆ heteroalkyl (a substituted or an unsubstituted C₁-C₆ heteroalkyl, a substituted or an unsubstituted C₁-C₄ heteroalkyl, a substituted or an unsubstituted C₁-C₃ heteroalkyl, a substituted or an unsubstituted C₁-C₂ heteroalkyl, etc.), a substituted or an unsubstituted C₁-C₆ alkoxy, a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.), a substituted or an unsubstituted 4- to 10-membered heterocyclyl (a substituted or an unsubstituted 4- to 10-membered heterocyclyl, a substituted or an unsubstituted 4- to 8-membered heterocyclyl, a substituted or an unsubstituted 5- to 8-membered heterocyclyl, a substituted or an unsubstituted 4- to 6-membered heterocyclyl, etc.), or a substituted or an unsubstituted 5- to 10-membered heteroaryl (a substituted or an unsubstituted 5- to 10-membered heteroaryl, a substituted or an unsubstituted 5- to 8-membered heteroaryl, a substituted or an unsubstituted 5- or 6-membered heteroaryl, a substituted or an unsubstituted 5-membered heteroaryl, a substituted or an unsubstituted 6-membered heteroaryl, etc.), wherein:
when a C₁-C₆ alkyl or a C₁-C₆ heteroalkyl represented by R¹, R², R⁵, R⁶, R⁷, R⁸, R⁹, R¹⁰, R¹¹, R¹², R¹⁴, or R¹⁵ is substituted, the group is substituted with one or more groups each independently selected from deuterium, halogen, —CN, —OH, C₁-C₆ alkyl, C₁-C₆ haloalkyl, C₁-C₆ alkoxy, and C₃-C₆ cycloalkyl;
when a C₃-C₆ cycloalkyl, a 4- to 10-membered heterocyclyl, or a 5- to 10-membered heteroaryl represented by R¹, R², R⁵, R⁶, R⁷, R⁸, R⁹, R¹⁰, R¹¹, R¹², R¹⁴, or R¹⁵ is substituted, the group is substituted with one or more groups each independently selected from halogen, —CN, —OH, C₁-C₆ alkyl, C₁-C₆ alkyl substituted with one or more groups each independently selected from C₁-C₄ alkoxy, C₁-C₆ haloalkyl, and C₁-C₆ alkoxy; or alternatively
R¹ and R², R⁵ and R⁶, R⁷ and R⁸, R⁹ and R¹⁰, R¹¹ and R¹², or R¹⁴ and R¹⁵, together with the respective nitrogen atom connected thereto, each independently form a substituted or an unsubstituted 4- to 10-membered heterocyclyl or a substituted or an unsubstituted 5- to 10-membered heteroaryl, wherein:
when a 4- to 10-membered heterocyclyl or a 5- to 10-membered heteroaryl formed by R¹ or R², R⁵ and R⁶, R⁷ and R⁸, R⁹ and R¹⁰, R¹¹ and R¹², or R¹⁴ and R¹⁵, together with the respective nitrogen atom connected thereto, is substituted, the group is substituted with one or more groups each independently selected from halogen, —CN, —OH, C₁-C₆ alkyl, C₁-C₆ alkyl substituted with one or more groups each independently selected from —OH, C₁-C₄ alkoxy, C₁-C₆ haloalkyl, and C₁-C₆ alkoxy;
and all other variables are as defined in any one of the hundredth-and-fifth through two-hundred-and-tenth embodiments.

In a hundredth-and-thirty-seventh embodiment, a compound of this disclosure is represented by the following Formula (IVa) or (IVa-1):

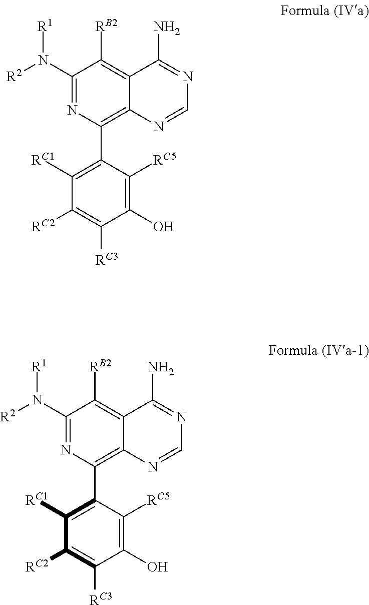

or a pharmaceutically acceptable salt thereof, and all other variables are as defined in any one of the hundredth-and-fifth through two-hundred-and-tenth embodiments.

In a hundredth-and-thirty-eighth embodiment, a compound of this disclosure is represented by the following Formula (V′a) or (V′a-1):

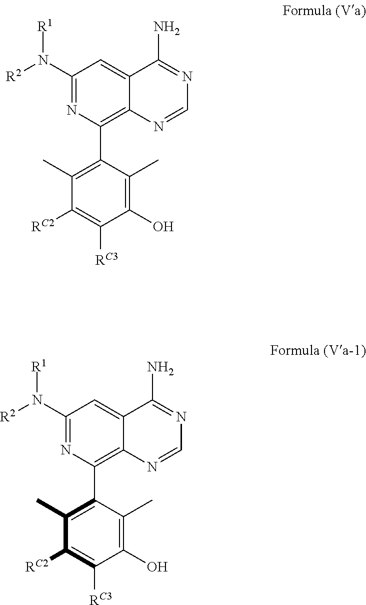

or a pharmaceutically acceptable salt thereof, and all other variables are as defined in any one of the hundredth-and-fifth through two-hundred-and-tenth embodiments.

In a hundredth-and-thirty-nineth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), (III′d-1), (IV′a), (IV′a-1), (V′a), or (V′a-1), or a pharmaceutically acceptable salt thereof, R¹ and R² are each independently hydrogen, a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁—C₂ alkyl, —CD₃, etc.), a substituted or an unsubstituted C₁-C₆ heteroalkyl (a substituted or an unsubstituted C₁-C₆ heteroalkyl, a substituted or an unsubstituted C₁-C₄ heteroalkyl, a substituted or an unsubstituted C₁-C₃ heteroalkyl, a substituted or an unsubstituted C₁-C₂ heteroalkyl, etc.), a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.), a substituted or an unsubstituted 4- to 10-membered heterocyclyl (a substituted or an unsubstituted 4- to 10-membered heterocyclyl, a substituted or an unsubstituted 4- to 8-membered heterocyclyl, a substituted or an unsubstituted 5- to 8-membered heterocyclyl, a substituted or an unsubstituted 4- to 6-membered heterocyclyl, etc., including a substituted or an unsubstituted fused bicyclic 6-membered heterocyclyl containing at least one oxygen atom), or a substituted or an unsubstituted 5- to 10-membered heteroaryl (a substituted or an unsubstituted 5- to 10-membered heteroaryl, a substituted or an unsubstituted 5- to 8-membered heteroaryl, a substituted or an unsubstituted 5- or 6-membered heteroaryl, a substituted or an unsubstituted 5-membered heteroaryl, a substituted or an unsubstituted 6-membered heteroaryl, etc.), wherein:
when a C₁-C₆ alkyl or a C₁-C₆ heteroalkyl represented by R or R² is substituted, the C₁-C₆ alkyl or the C₁-C₆ heteroalkyl is substituted with one or more groups each independently selected from deuterium, halogen, —CN, —OH, C₁-C₄ alkyl, C₁-C₄ haloalkyl, C₁-C₄ alkoxy, and C₃-C₆ cycloalkyl;
when a C₃-C₆ cycloalkyl, a 4- to 10-membered heterocyclyl, or a 5- to 10-membered heteroaryl represented by R¹ or R² is substituted, the C₃-C₆ cycloalkyl, the 4- to 10-membered heterocyclyl, or the 5- to 10-membered heteroaryl is substituted with one or more groups each independently selected from halogen, —CN, —OH, C₁-C₄ alkyl, C₁-C₄ alkyl substituted with one or more groups each independently selected from C₁-C₂ alkoxy, C₁-C₄ haloalkyl, and C₁-C₄ alkoxy; or alternatively
R¹ and R², together with the nitrogen atom connected thereto, form a substituted or an unsubstituted 4- to 8-membered heterocyclyl (a substituted or an unsubstituted 4- to 8-membered heterocyclyl, a substituted or an unsubstituted 5- to 8-membered heterocyclyl, a substituted or an unsubstituted 4- to 6-membered heterocyclyl, etc.) or a substituted or an unsubstituted 5- to 8-membered heteroaryl (a substituted or an unsubstituted 5- to 8-membered heteroaryl, a substituted or an unsubstituted 5- or 6-membered heteroaryl, a substituted or an unsubstituted 5-membered heteroaryl, or a substituted or an unsubstituted 6-membered heteroaryl, etc.), wherein:
when a 4- to 8-membered heterocyclyl or a 5- to 8-membered heteroaryl formed by R¹ or R², together with the nitrogen atom connected thereto, is substituted, the 4- to 8-membered heterocyclyl or the 5- to 8-membered heteroaryl is substituted with one or more groups each independently selected from halogen, —CN, —OH, C₁-C₄ alkyl, C₁-C₄ alkyl substituted with one or more groups each independently selected from —OH, C₁-C₂ alkoxy, C₁-C₄ haloalkyl, and C₁-C₄ alkoxy;
and all other variables are as defined in any one of the hundredth-and-fifth through two-hundred-and-tenth embodiments.

In a hundredth-and-forty embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), (III′d-1), (IV′a), (IV′a-1), (V′a), or (V′a-1), or a pharmaceutically acceptable salt thereof, R¹ and R² are each independently hydrogen, a substituted or an unsubstituted C₁-C₄ alkyl, a substituted or an unsubstituted C₃-C₅ cycloalkyl, a substituted or an unsubstituted 4- to 8-membered heterocyclyl (e.g., a substituted or an unsubstituted fused bicyclic 6-membered heterocyclyl containing at least one oxygen atom), or a substituted or an unsubstituted 5- to 8-membered heteroaryl, wherein:
when a C₁-C₄ alkyl represented by R¹ or R² is substituted, the C₁-C₄ alkyl is substituted with one or more groups each independently selected from D (deuterium), F, Cl, —CN, —OH, C₁-C₂ alkyl, C₁-C₂ haloalkyl, C₁-C₂ alkoxy, and C₃-C₄ cycloalkyl;
when a C₃-C₅ cycloalkyl, a 4- to 8-membered heterocyclyl, or a 5- to 8-membered heteroaryl represented by R¹ or R² is substituted, the C₃-C₅ cycloalkyl, the 4- to 8-membered heterocyclyl, or the 5- to 8-membered heteroaryl is substituted with one or more groups each independently selected from F, Cl, —CN, —OH, C₁-C₂ alkyl, C₁-C₂ alkyl substituted with one or more groups each independently selected from C₁-C₂ alkoxy, C₁-C₂ haloalkyl, and C₁-C₂ alkoxy; or alternatively
R¹ and R², together with the nitrogen atom connected thereto, form a substituted or an unsubstituted 4- to 8-membered heterocyclyl or a substituted or an unsubstituted 5- or 6-membered heteroaryl, wherein:
when a 4- to 8-membered heterocyclyl or a 5- or 6-membered heteroaryl formed by R¹ or R², together with the nitrogen atom connected thereto, is substituted, the 4- to 8-membered heterocyclyl or the 5- or 6-membered heteroaryl is substituted with one or more groups each independently selected from F, Cl, —CN, —OH, C₁-C₂ alkyl, C₁-C₂ alkyl substituted with one or more groups each independently selected from —OH, C₁-C₂ alkoxy, C₁-C₂ haloalkyl, and C₁-C₂ alkoxy;
and all other variables are as defined in any one of the hundredth-and-fifth through two-hundred-and-tenth embodiments. In some embodiments of the hundredth-and-forty embodiment, R¹ can be H or an unsubstituted C₁-C₄ alkyl; and R² can be an unsubstituted C₁-C₄ alkyl or an unsubstituted C₃-C₅ cycloalkyl. In some embodiments of the hundredth-and-forty embodiment, R¹ can be H or an unsubstituted C₁-C₄ alkyl; and R² can be an substituted C₁-C₄ alkyl substituted with one or more (e.g., 1, 2 or 3) groups each independently selected from F, —CN, —OH and C₃-C₄ cycloalkyl, a substituted C₃-C₅ cycloalkyl substituted with one or more (e.g., 1, 2 or 3) groups each independently selected from —OH, F and C₁-C₂ alkoxy, a substituted or an unsubstituted 4- to 8-membered heterocyclyl (e.g., a substituted or an unsubstituted fused bicyclic 6-membered heterocyclyl containing at least one oxygen atom such as 3-oxabicyclo[3.1.0]hexane) or a substituted or an unsubstituted 5- to 6-membered monocyclic heteroaryl (such as a substituted or an unsubstituted pyrrolyl, including a C₁-C₂ alkyl substituted pyrrolyl). In some embodiments of the hundredth-and-forty embodiment, R¹ and R², together with the nitrogen atom connected thereto, form a substituted or an unsubstituted 4- to 8-membered heterocyclyl (e.g., a substituted or an unsubstituted 4- to 8-membered spiro-connected heterocyclyl including a substituted or an unsubstituted 4- to 8-membered spiro-connected heterocyclyl containing at least one oxygen atom such as 1-oxa-6-azaspiro[3.4]octane).

In a hundredth-and-forty-first embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), (III′d-1), (IV′a), (IV′a-1), (V′a), or (V′a-1), or a pharmaceutically acceptable salt thereof, R¹ and R², together with the nitrogen atom connected thereto, form a substituted or an unsubstituted 5- to 6-membered heterocyclyl (e.g., a substituted or an unsubstituted 5-membered heterocyclyl containing at least one nitrogen atom or a substituted or an unsubstituted 6-membered heterocyclyl containing at least one nitrogen atom and/or at least one oxygen atom and optionally at least one double bond in the heterocyclyl) or a substituted or an unsubstituted 5- or 6-membered heteroaryl, wherein:
when a 5- to 6-membered heterocyclyl or a 5- or 6-membered heteroaryl formed by R¹ or R², together with the nitrogen atom connected thereto, is substituted, the 5- to 6-membered heterocyclyl or the 5- or 6-membered heteroaryl is substituted with one or more groups each independently selected from F, Cl, —CN, —OH, C₁-C₂ alkyl, C₁-C₂ alkyl substituted with one or more groups each independently selected from —OH, C₁-C₂ alkoxy, C₁-C₂ haloalkyl, and C₁-C₂ alkoxy;
and all other variables are as defined in any one of the hundredth-and-fifth through two-hundred-and-tenth embodiments. In some embodiments of the hundredth-and-forty-first embodiment, R¹ and R², together with the nitrogen atom connected thereto, form a substituted or an unsubstituted 5- to 6-membered monocyclic heterocyclyl (such as azetidinyl, pyrrolidinyl, pyrrolidin-2-one, piperidinyl and morpholinyl), wherein the substituted 5- to 6-membered monocyclic heterocyclyl is substituted with one or more groups (e.g., 1, 2 or 3) each independently selected from F, —CN, —OH, C₁-C₂ alkyl, C₁-C₂ alkyl substituted with one or more groups each independently selected from —OH, C₁-C₂ alkoxy, C₁-C₂ haloalkyl, and C₁-C₂ alkoxy.

In a hundredth-and-forty-second embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

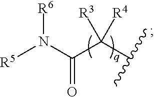

and all other variables are as defined in any one of the hundredth-and-fifth through two-hundred-and-tenth embodiments.

In a hundredth-and-forty-third embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

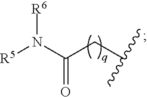

and all other variables are as defined in any one of the hundredth-and-fifth through two-hundred-and-tenth embodiments. In some embodiments of the hundredth-and-forty-third embodiment, R⁵ and R⁶, together with the nitrogen atom connected thereto, form a substituted or an unsubstituted 4- to 6-membered monocyclic heterocyclyl (for example, a substituted or an unsubstituted pyrrolidinyl and a substituted or an unsubstituted morpholinyl). In some embodiments of the hundredth-and-forty-third embodiment, R⁵ and R⁶, together with the nitrogen atom connected thereto, form an unsubstituted 4- to 6-membered monocyclic heterocyclyl (for example, an unsubstituted pyrrolidinyl). In some embodiments of the forty-second embodiment, q is 1.

In a hundredth-and-forty-forth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

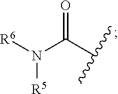

and all other variables are as defined in any one of the hundredth-and-fifth through two-hundred-and-tenth embodiments. In some embodiments of the hundredth-and-forty-forth embodiment, R⁵ and R⁶, together with the nitrogen atom connected thereto, form a substituted or an unsubstituted 4- to 6-membered monocyclic heterocyclyl (for example, a substituted or an unsubstituted pyrrolidinyl and a substituted or an unsubstituted morpholinyl). In some embodiments of the hundredth-and-forty-forth embodiment, R⁵ and R⁶, together with the nitrogen atom connected thereto, form an unsubstituted 4- to 6-membered monocyclic heterocyclyl (for example, an unsubstituted pyrrolidinyl).

In a hundredth-and-forty-fifth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R⁵ and R⁶ are each independently hydrogen or a substituted or an unsubstituted C₁-C₆ alkyl, wherein:
when a C₁-C₆ alkyl represented by R⁵ or R⁶ is substituted, the C₁-C₆ alkyl is substituted with one or more groups each independently selected from deuterium, halogen, —CN, —OH, and C₁-C₄ alkoxy; or alternatively
R⁵ and R⁶, together with the nitrogen atom connected thereto, form a substituted or an unsubstituted 4- to 8-membered heterocyclyl, wherein:
when a 4- to 8-membered heterocyclyl formed by R⁵ or R⁶, together with the nitrogen atom connected thereto, is substituted, the 4- to 8-membered heterocyclyl is substituted with one or more groups each independently selected from halogen, —CN, —OH, C₁-C₄ alkyl, C₁-C₄ alkyl substituted with one or more groups each independently selected from —OH, C₁-C₂ alkoxy, C₁-C₄ haloalkyl, and C₁-C₄ alkoxy;
and all other variables are as defined in any one of the hundredth-and-fifth through two-hundred-and-tenth embodiments.

In a hundredth-and-forty-sixth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R⁵ and R⁶ are each independently hydrogen or a substituted or an unsubstituted C₁-C₄ alkyl, wherein:
when a C₁-C₄ alkyl represented by R⁵ or R⁶ is substituted, the C₁-C₄ alkyl is substituted with one or more groups each independently selected from D (deuterium), F, Cl, —CN, —OH, and C₁-C₂ alkoxy; or alternatively
R⁵ and R⁶, together with the nitrogen atom connected thereto, form a substituted or an unsubstituted 4- to 6-membered heterocyclyl, wherein:
when a 4- to 6-membered heterocyclyl formed by R⁵ or R⁶, together with the nitrogen atom connected thereto, is substituted, the 4- to 6-membered heterocyclyl is substituted with one or more groups each independently selected from F, Cl, —CN, —OH, C₁-C₂ alkyl, C₁-C₂ alkyl substituted with one or more groups each independently selected from —OH, C₁-C₂ alkoxy, C₁-C₂ haloalkyl, and C₁-C₂ alkoxy;
and all other variables are as defined in any one of the hundredth-and-fifth through two-hundred-and-tenth embodiments.

In a hundredth-and-forty-seventh embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

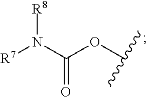

and all other variables are as defined in any one of the hundredth-and-fifth through two-hundred-and-tenth embodiments.

In a hundredth-and-forty-eighth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

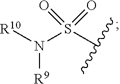

and all other variables are as defined in any one of the hundredth-and-fifth through two-hundred-and-tenth embodiments.

In a hundredth-and-forty-ninth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R¹⁵, R¹⁶, R¹⁷, R¹⁹, and R²¹ are each independently hydrogen, a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁-C₂ alkyl, —CD₃, etc.), a substituted or an unsubstituted C₁-C₆ alkoxy (a substituted or an unsubstituted C₁-C₆ alkoxy, a substituted or an unsubstituted C₁-C₄ alkoxy, a substituted or an unsubstituted C₁-C₃ alkoxy, a substituted or an unsubstituted C₁-C₂ alkoxy, etc.), or a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.); and all other variables are as defined in any one of the hundredth-and-fifth through two-hundred-and-tenth embodiments.

In a hundredth-and-fifty embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

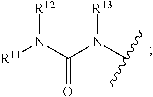

and all other variables are as defined in any one of the hundredth-and-fifth through two-hundred-and-tenth embodiments.

In a hundredth-and-fifty-first embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

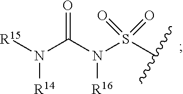

and all other variables are as defined in any one of the hundredth-and-fifth through two-hundred-and-tenth embodiments.

In a hundredth-and-fifty-second embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R¹⁸, R²⁰, R²³, R²⁴, R²⁵, and R²⁶ are each independently hydrogen, a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁-C₂ alkyl, —CD₃, etc.), or a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.); and all other variables are as defined in any one of the hundredth-and-fifth through two-hundred-and-tenth embodiments.

In a hundredth-and-fifty-third embodiment, in a compound represented by Formula (I′), (I′a), (I′b), (I′c), (I′d), (II′), (II′-1), (III′a), (III′b), (III′c), (III′d), (II′a-1), (III′b-1), (III′c-1), or (III′d-1), or a pharmaceutically acceptable salt thereof, Z is

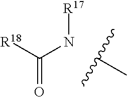

and all other variables are as defined in any one of the hundredth-and-fifth through two-hundred-and-tenth embodiments.

In a hundredth-and-fifty-forth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R¹⁷ is hydrogen or a substituted or an unsubstituted C₁-C₆ alkyl; and R¹⁸ is a substituted or an unsubstituted C₁-C₆ alkyl, wherein:
when a C₁-C₆ alkyl represented by R¹⁷ or R¹⁸ is substituted, the C₁-C₆ alkyl is substituted with one or more groups each independently selected from deuterium, halogen, —CN, —OH, and C₁-C₄ alkoxy;
and all other variables are as defined in any one of the hundredth-and-fifth through two-hundred-and-tenth embodiments.

In a hundredth-and-fifty-fifth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R¹⁷ is hydrogen or a substituted or an unsubstituted C₁-C₄ alkyl; and R¹⁸ is a substituted or an unsubstituted C₁-C₄ alkyl, wherein:
when a C₁-C₄ alkyl represented by R¹⁷ or R¹⁸ is substituted, the C₁-C₄ alkyl is substituted with one or more groups each independently selected from D (deuterium), F, Cl, —CN, —OH, and C₁-C₂ alkoxy;
and all other variables are as defined in any one of the hundredth-and-fifth through two-hundred-and-tenth embodiments.

In a hundredth-and-fifty-sixth embodiment, in a compound represented by Formula (I), (Ia), (Ib), (Ic), (Id), (II), (II-1), (IIIa), (IIIb), (IIIc), (IIId), (IIIa-1), (IIIb-1), (IIIc-1), or (IIId-1), or a pharmaceutically acceptable salt thereof, Z is

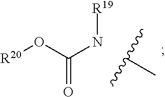

and all other variables are as defined in any one of the hundredth-and-fifth through two-hundred-and-tenth embodiments.

In a hundredth-and-fifty-seventh embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R²², R²⁷, R²⁸, R²⁹, R³⁰, and R³¹ are each independently a substituted or an unsubstituted C₁-C₆ alkyl (a substituted or an unsubstituted C₁-C₆ alkyl, a substituted or an unsubstituted C₁-C₄ alkyl, a substituted or an unsubstituted C₁-C₃ alkyl, a substituted or an unsubstituted C₁-C₂ alkyl, etc., including deuterated C₁-C₆ alkyl, C₁-C₄ alkyl, C₁-C₃ alkyl, C₁-C₂ alkyl, —CD₃, etc.), a substituted or an unsubstituted C₁-C₆ alkoxy (a substituted or an unsubstituted C₁-C₆ alkoxy, a substituted or an unsubstituted C₁-C₄ alkoxy, a substituted or an unsubstituted C₁-C₃ alkoxy, a substituted or an unsubstituted C₁-C₂ alkoxy, etc.), or a substituted or an unsubstituted C₃-C₆ cycloalkyl (a substituted or an unsubstituted C₃-C₆ cycloalkyl, a substituted or an unsubstituted C₃-C₅ cycloalkyl, a substituted or an unsubstituted C₃-C₄ cycloalkyl, etc.); and all other variables are as defined in any one of the hundredth-and-fifth through two-hundred-and-tenth embodiments.

In a hundredth-and-fifty-eighth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

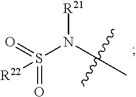

and all other variables are as defined in any one of the hundredth-and-fifth through two-hundred-and-tenth embodiments.

In a hundredth-and-fifty-nineth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R²¹ is hydrogen or a substituted or an unsubstituted C₁-C₆ alkyl; and R²² is a substituted or an unsubstituted C₁-C₆ alkyl, wherein:
when a C₁-C₆ alkyl represented by R²¹ or R²² is substituted, the C₁-C₆ alkyl is substituted with one or more groups each independently selected from deuterium, halogen, —CN, —OH, and C₁-C₄ alkoxy;
and all other variables are as defined in any one of the hundredth-and-fifth through two-hundred-and-tenth embodiments.

In a hundredth-and-sixty embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R²¹ is hydrogen or a substituted or an unsubstituted C₁-C₄ alkyl; and R²² is a substituted or an unsubstituted C₁-C₄ alkyl, wherein:
when a C₁-C₄ alkyl represented by R²¹ or R²² is substituted, the C₁-C₄ alkyl is substituted with one or more groups each independently selected from D (deuterium), F, Cl, —CN, —OH, and C₁-C₂ alkoxy;
and all other variables are as defined in any one of the hundredth-and-fifth through two-hundred-and-tenth embodiments. In some embodiments of the hundredth-and-sixty embodiment, R²¹ is hydrogen; and R²² is an unsubstituted C₁-C₄ alkyl (e.g., methyl).

In a hundredth-and-sixty-first embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

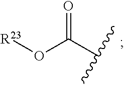

and all other variables are as defined in any one of the hundredth-and-fifth through two-hundred-and-tenth embodiments.

In a hundredth-and-sixty-second embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

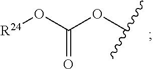

and all other variables are as defined in any one of the hundredth-and-fifth through two-hundred-and-tenth embodiments.

In a hundredth-and-sixty-third embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

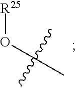

and all other variables are as defined in any one of the hundredth-and-fifth through two-hundred-and-tenth embodiments.

In a hundredth-and-sixty-forth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R²⁵ is a substituted or an unsubstituted C₁-C₆ alkyl, wherein:
when a C₁-C₆ alkyl represented by R²⁵ is substituted, the C₁-C₆ alkyl is substituted with one or more groups each independently selected from deuterium, halogen, —CN, —OH, and C₁-C₄ alkoxy;
and all other variables are as defined in any one of the hundredth-and-fifth through two-hundred-and-tenth embodiments.

In a hundredth-and-sixty-fifth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R²⁵ is a substituted or an unsubstituted C₁-C₄ alkyl, wherein:
when a C₁-C₄ alkyl represented by R²⁵ is substituted, the C₁-C₄ alkyl is substituted with one or more groups each independently selected from D (deuterium), F, Cl, —CN, —OH, and C₁-C₂ alkoxy;
and all other variables are as defined in any one of the hundredth-and-fifth through two-hundred-and-tenth embodiments. In some embodiments of the hundredth-and-sixty-fifth embodiment, R²⁵ is an unsubstituted C₁-C₄ alkyl (e.g., methyl).

In a hundredth-and-sixty-sixth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

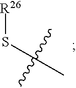

and all other variables are as defined in any one of the hundredth-and-fifth through two-hundred-and-tenth embodiments.

In a hundredth-and-sixty-seventh embodiment, in a compound represented by Formula (I′), (I′a), (I′b), (I′c), (I′d), (II′), (II′-1), (III′a), (III′b), (III′c), (III′d), (II′a-1), (III′b-1), (III′c-1), or (III′d-1), or a pharmaceutically acceptable salt thereof, Z is

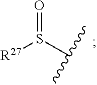

and all other variables are as defined in any one of the hundredth-and-fifth through two-hundred-and-tenth embodiments.

In a hundredth-and-sixty-eighth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

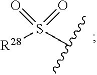

and all other variables are as defined in any one of the hundredth-and-fifth through two-hundred-and-tenth embodiments.

In a hundredth-and-sixty-nineth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

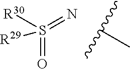

and all other variables are as defined in any one of the hundredth-and-fifth through two-hundred-and-tenth embodiments.

In a hundredth-and-seventy embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R²⁹ and R³⁰ are each a substituted or an unsubstituted C₁-C₆ alkyl, wherein:
when a C₁-C₆ alkyl represented by R²⁹ or R³⁰ is substituted, the C₁-C₆ alkyl is substituted with one or more groups each independently selected from deuterium, halogen, —CN, —OH, and C₁-C₄ alkoxy;
and all other variables are as defined in any one of the hundredth-and-fifth through two-hundred-and-tenth embodiments.

In a hundredth-and-seventy-first embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R²⁹ and R³⁰ are each a substituted or an unsubstituted C₁-C₄ alkyl, wherein:
when a C₁-C₄ alkyl represented by R²⁹ or R³⁰ is substituted, the C₁-C₄ alkyl is substituted with one or more groups each independently selected from D (deuterium), F, Cl, —CN, —OH, and C₁-C₂ alkoxy;
and all other variables are as defined in any one of the hundredth-and-fifth through two-hundred-and-tenth embodiments. In some embodiments of the sixty-seventh embodiment, R²⁹ and R³⁰ are each an unsubstituted C₁-C₄ alkyl (e.g., methyl).

In a hundredth-and-seventy-second embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R³² is hydrogen, —CN, a substituted or an unsubstituted C₁-C₆ alkyl, a substituted or an unsubstituted C₁-C₆ alkoxy, or a substituted or an unsubstituted C₃-C₆ cycloalkyl; and all other variables are as defined in any one of the hundredth-and-fifth through two-hundred-and-tenth embodiments.

In a hundredth-and-seventy-third embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R³³ and R³⁴ are each independently hydrogen, a substituted or an unsubstituted C₁-C₆ alkyl, a substituted or an unsubstituted C₁-C₆ alkoxy, or a substituted or an unsubstituted C₃-C₆ cycloalkyl; and all other variables are as defined in any one of the hundredth-and-fifth through two-hundred-and-tenth embodiments.

In a hundredth-and-seventy-forth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

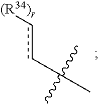

and all other variables are as defined in any one of the hundredth-and-fifth through two-hundred-and-tenth embodiments.

In a hundredth-and-seventy-fifth embodiment, in a compound represented by Formula (I′), (I′a), (I′b), (I′c), (I′d), (II′), (II′-1), (III′a), (III′b), (III′c), (III′d), (II′a-1), (III′b-1), (III′c-1), or (III′d-1), or a pharmaceutically acceptable salt thereof, Z is

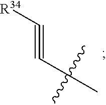

and all other variables are as defined in any one of the hundredth-and-fifth through two-hundred-and-tenth embodiments.

In a hundredth-and-seventy-sixth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is

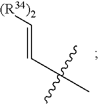

and all other variables are as defined in any one of the hundredth-and-fifth through two-hundred-and-tenth embodiments.

In a hundredth-and-seventy-seventh embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R³⁴ is hydrogen, a substituted or an unsubstituted C₁-C₆ alkyl, or C₁-C₆ alkoxy, wherein: when a C₁-C₆ alkyl represented by R³⁴ is substituted, the C₁-C₆ alkyl is substituted with one or more groups each independently selected from deuterium, halogen, —CN, —OH, and C₁-C₄ alkoxy;
and all other variables are as defined in any one of the hundredth-and-fifth through two-hundred-and-tenth embodiments.

In a hundredth-and-seventy-eighth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, R³⁴ is hydrogen, a substituted or an unsubstituted C₁-C₄ alkyl, or C₁-C₄ alkoxy, wherein: when a C₁-C₄ alkyl represented by R³⁴ is substituted, the C₁-C₄ alkyl is substituted with one or more groups each independently selected from D (deuterium), F, Cl, —CN, —OH, and C₁-C₂ alkoxy; and all other variables are as defined in any one of the hundredth-and-fifth through two-hundred-and-tenth embodiments. In some embodiments of the hundredth-and-seventy-eighth embodiment, R³⁴ is hydrogen.

In a hundredth-and-seventy-nineth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is halogen, and all other variables are as defined in any one of the hundredth-and-fifth through two-hundred-and-tenth embodiments.

In a hundredth-and-eighty embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is F, and all other variables are as defined in any one of the hundredth-and-fifth through two-hundred-and-tenth embodiments.

In a hundredth-and-eighty-first embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is Cl, and all other variables are as defined in any one of the hundredth-and-fifth through two-hundred-and-tenth embodiments.

In a hundredth-and-eighty-second embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, with the exception of Formula (I′) where X is —CR^{B1}; X² is —CR^{B2}; Y¹ is —CR^{C2}; and Y² is —CR^{C3}, and Formula (Ib) where Y¹ is —CR^{C2}; Y² is —CR^{C3}, and Z is halogen, and all other variables are as defined in any one of the hundredth-and-fifth through two-hundred-and-tenth embodiments.

In a hundredth-and-eighty-third embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, with the exception of Formula (I′) where X is —CR^{B1}; X² is —CR^{B2}; Y¹ is —CR^{C2}; and Y² is —CR^{C3}, and Formula (I′b) where Y¹ is —CR^{C2}; Y² is —CR^{C3}, and Z is F, and all other variables are as defined in any one of the hundredth-and-fifth through two-hundred-and-tenth embodiments.

In a hundredth-and-eighty-forth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, with the exception of Formula (I′) where X¹ is —CR^{B1}; X² is —CR^{B2}; Y¹ is —CR^{C2}; and Y² is —CR^{C3}, and Formula (I′b) where Y¹ is —CR^{C2}; Y² are each —CR^{C3}, and Z is Cl, and all other variables are as defined in any one of the hundredth-and-fifth through two-hundred-and-tenth embodiments.

In a hundredth-and-eighty-fifth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is —CN, and all other variables are as defined in any one of the hundredth-and-fifth through two-hundred-and-tenth embodiments.

In a hundredth-and-eighty-sixth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted C₁-C₆ alkyl; and all other variables are as defined in any one of the hundredth-and-fifth through two-hundred-and-tenth embodiments.

In a hundredth-and-eighty-seventh embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted C₁-C₆ alkyl, wherein:
when a C₁-C₆ alkyl represented by Z is substituted, the C₁-C₆ alkyl is substituted with one or more groups each independently selected from deuterium, halogen, —CN, —OH, C₁-C₄ alkoxy, a C₃-C₆ cycloalkyl, and a 4- to 8-membered heterocyclyl;
and all other variables are as defined in any one of the hundredth-and-fifth through two-hundred-and-tenth embodiments.

In a hundredth-and-eighty-eighth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted C₁-C₄ alkyl, wherein:
when a C₁-C₄ alkyl represented by Z is substituted, the C₁-C₄ alkyl is substituted with one or more groups each independently selected from D (deuterium), F, Cl, —CN, —OH, C₁-C₂ alkoxy, a C₃-C₅ cycloalkyl, and a 5- or 6-membered heterocyclyl;
and all other variables are as defined in any one of the hundredth-and-fifth through two-hundred-and-tenth embodiments. In some embodiments of the hundredth-and-eighty-eighth embodiment, Z is an unsubstituted C₁-C₄ alkyl (e.g., methyl, ethyl, n-propyl, iso-propyl, n-butyl, iso-butyl, sec-butyl and tert-butyl). In some embodiments of the hundredth-and-eighty-eighth embodiment, Z is a substituted C₁-C₄ alkyl substituted with one or more (e.g., 1, 2, 3, 4 or 5) F. For example, Z is —CH₂F, —CHF₂, —CF₃, —CH₂CH₂F, —CH₂CHF₂, —CH₂CF₃.

In a hundredth-and-eighty-nineth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted C₂-C₆ alkenyl (e.g., a C₂-C₆ alkenyl optionally substituted with one or more groups selected from halogen, —CN, —OH, and C₁-C₂ alkoxy, etc.); and all other variables are as defined in any one of the hundredth-and-fifth through two-hundred-and-tenth embodiments.

In a hundredth-and-ninety embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted C₂-C₆ alkenyl (e.g., C₂-C₆ alkenyl, C₂-C₄ alkenyl), wherein:
when a C₂-C₆ alkenyl (e.g., C₂-C₆ alkenyl, C₂-C₄ alkenyl) represented by Z is substituted, the C₂-C₆ alkenyl (e.g., C₂-C₆ alkenyl, C₂-C₄ alkenyl) is substituted with one or more groups each independently selected from halogen, —CN, —OH, C₁-C₄ alkoxy, a C₃-C₆ cycloalkyl, and a 4- to 8-membered heterocyclyl;
and all other variables are as defined in any one of the hundredth-and-fifth through two-hundred-and-tenth embodiments. In some embodiments of the hundredth-and-ninety embodiment, Z is an unsubstituted C₂-C₆ alkenyl (e.g., C₂-C₆ alkenyl, C₂-C₄ alkenyl).

In a hundredth-and-ninety-first embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted C₂-C₆ alkynyl (e.g., a C₂-C₆ alkynyl optionally substituted with one or more groups selected from halogen, —CN, —OH, and C₁-C₂ alkoxy, etc.); and all other variables are as defined in any one of the hundredth-and-fifth through two-hundred-and-tenth embodiments. In some embodiments of the hundredth-and-ninety-first embodiment, Z is an unsubstituted C₂-C₆ alkynyl.

In a hundredth-and-ninety-second embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted C₃-C₆ cycloalkyl; and all other variables are as defined in any one of the hundredth-and-fifth through two-hundred-and-tenth embodiments.

In a hundredth-and-ninety-third embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted C₃-C₆ cycloalkyl, wherein:
when a C₃-C₆ cycloalkyl represented by Z is substituted, the C₃-C₆ cycloalkyl is substituted with one or more groups each independently selected from halogen, —CN, —OH, C₁-C₆ alkyl, C₁-C₆ alkyl substituted with one or more groups each independently selected from —OH, C₁-C₄ alkoxy, C₁-C₆ haloalkyl, and C₁-C₆ alkoxy;
and all other variables are as defined in any one of the hundredth-and-fifth through two-hundred-and-tenth embodiments.

In a hundredth-and-ninety-forth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted C₃-C₅ cycloalkyl, wherein:
when a C₃-C₅ cycloalkyl represented by Z is substituted, the C₃-C₅ cycloalkyl is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₄haloalkyl, and C₁-C₄ alkoxy;
and all other variables are as defined in any one of the hundredth-and-fifth through two-hundred-and-tenth embodiments. In some embodiments of the hundredth-and-ninety-forth embodiment, Z is a substituted or an unsubstituted monocyclic C₃-C₅ cycloalkyl (e.g., cyclopropyl, cyclobutyl and cyclopentyl). In some embodiments of the hundredth-and-ninety-forth embodiment, Z is an unsubstituted monocyclic C₃-C₅ cycloalkyl.

In a hundredth-and-ninety-fifth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted phenyl; and all other variables are as defined in any one of the hundredth-and-fifth through two-hundred-and-tenth embodiments.

In a hundredth-and-ninety-sixth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted phenyl, wherein:
when a phenyl represented by Z is substituted, the phenyl is substituted with one or more groups each independently selected from halogen, —CN, —OH, C₁-C₆ alkyl, C₁-C₆ alkyl substituted with one or more groups each independently selected from —OH, C₁-C₄ alkoxy, C₁-C₆ haloalkyl, and C₁-C₆ alkoxy;
and all other variables are as defined in any one of the hundredth-and-fifth through two-hundred-and-tenth embodiments.

In a hundredth-and-ninety-seventh embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted phenyl, wherein:
when a phenyl represented by Z is substituted, the phenyl is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₆ haloalkyl, and C₁-C₄ alkoxy;
and all other variables are as defined in any one of the hundredth-and-fifth through two-hundred-and-tenth embodiments. In some embodiments of the hundredth-and-ninety-seventh embodiment, Z is a substituted phenyl such as a phenyl substituted 1, 2, 3, 4 or 5 times with halogen (e.g., F).

In a hundredth-and-ninety-eighth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted 4- to 10-membered heterocyclyl; and all other variables are as defined in any one of the hundredth-and-fifth through two-hundred-and-tenth embodiments.

In a hundredth-and-ninety-nineth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted 4- to 10-membered heterocyclyl, wherein:
when a 4- to 10-membered heterocyclyl represented by Z is substituted, the 4- to 10-membered heterocyclyl is substituted with one or more groups each independently selected from halogen, —CN, —OH, C₁-C₆ alkyl, C₁-C₆ alkyl substituted with one or more groups each independently selected from —OH, C₁-C₄ alkoxy, C₁-C₆ haloalkyl, and C₁-C₆ alkoxy;
and all other variables are as defined in any one of the hundredth-and-fifth through two-hundred-and-tenth embodiments.

In a two-hundredth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted 4- to 10-membered heterocyclyl, or a substituted or an unsubstituted 4- to 8-membered heterocyclyl, or a substituted or an unsubstituted 5- to 6-membered heterocyclyl, or a substituted or an unsubstituted 5-membered heterocyclyl, or a substituted or an unsubstituted 6-membered heterocyclyl (e.g., a substituted or an unsubstituted 6-membered heterocyclyl containing a double bond and/or at least one oxygen atom), wherein: when a 4- to 10-membered heterocyclyl, or a substituted or an unsubstituted 4- to 8-membered heterocyclyl, or a substituted or an unsubstituted 5- to 6-membered heterocyclyl, or a substituted or an unsubstituted 5-membered heterocyclyl, or a substituted or an unsubstituted 6-membered heterocyclyl (e.g., a substituted or an unsubstituted 6-membered heterocyclyl containing a double bond and/or at least one oxygen atom) represented by Z is substituted, the 4- to 10-membered heterocyclyl, or the substituted or an unsubstituted 4- to 8-membered heterocyclyl, or the substituted or an unsubstituted 5- to 6-membered heterocyclyl, or the substituted or an unsubstituted 5-membered heterocyclyl, or the substituted or an unsubstituted 6-membered heterocyclyl (e.g., a substituted or an unsubstituted 6-membered heterocyclyl containing a double bond and/or at least one oxygen atom) is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₄haloalkyl (e.g., —CH₂F, —CHF₂, —CF₃, —CH₂CH₂F, —CH₂CHF₂, —CH₂CF₃), and C₁-C₄ alkoxy;
and all other variables are as defined in any one of the hundredth-and-fifth through two-hundred-and-tenth embodiments. For example, Z can be substituted azetidinyl, unsubstituted azetidinyl, substituted tetrahydrofuranyl, unsubstituted tetrahydrofuranyl, substituted pyrrolidinyl and unsubstituted pyrrolidinyl, substituted piperidinyl, unsubstituted piperidinyl, substituted morpholinyl, unsubstituted morpholinyl, substituted 3,6-dihydro-2H-pyranyl, unsubstituted 3,6-dihydro-2H-pyranyl, substituted 2,5-dihydrofuranyl, unsubstituted 2,5-dihydrofuranyl, substituted 3-oxabicyclo[3.1.0]hexane, unsubstituted 3-oxabicyclo[3.1.0]hexane, substituted 1-oxa-6-azaspiro[3.4]octane or unsubstituted 1-oxa-6-azaspiro[3.4]octane.

In a two-hundred-and-first embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted 4- to 10-membered heterocyclyl, wherein:
when a 4- to 10-membered heterocyclyl represented by Z is substituted, the 4- to 10-membered heterocyclyl is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₄ haloalkyl (e.g., —CH₂F, —CHF₂, —CF₃, —CH₂CH₂F, —CH₂CHF₂, —CH₂CF₃), and C₁-C₄ alkoxy;
and all other variables are as defined in any one of the hundredth-and-fifth through two-hundred-and-tenth embodiments.

In a two-hundred-and-second embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted 5- to 10-membered heteroaryl; and all other variables are as defined in any one of the hundredth-and-fifth through two-hundred-and-tenth embodiments. For example, Z can be substituted or unsubstituted pyridinyl, substituted or unsubstituted pyrazolyl, substituted or unsubstituted thiazolyl, substituted or unsubstituted isothiazolyl, substituted or unsubstituted imidazolyl, etc.).

In a two-hundred-and-third embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted 5- to 10-membered heteroaryl, wherein:
when a 5- to 10-membered heteroaryl represented by Z is substituted, the 5- to 10-membered heteroaryl is substituted with one or more groups each independently selected from halogen (e.g., F, Cl), —CN, —OH, C₁-C₆ alkyl, C₁-C₆ alkyl substituted with one or more groups each independently selected from —OH, C₁-C₄ alkoxy, C₁-C₆ haloalkyl (e.g., —CH₂F, —CHF₂, —CF₃, —CH₂CH₂F, —CH₂CHF₂, —CH₂CF₃), and C₁-C₆ alkoxy;
and all other variables are as defined in any one of the hundredth-and-fifth through two-hundred-and-tenth embodiments.

In a two-hundred-and-forth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), or (III′d-1), or a pharmaceutically acceptable salt thereof, Z is a substituted or an unsubstituted 5- to 10-membered heteroaryl, wherein:
when a 5- to 10-membered heteroaryl represented by Z is substituted, the 5- to 10-membered heteroaryl is substituted with one or more groups each independently selected from halogen (e.g., F, Cl), —CN, —OH, C₁-C₄ alkyl, C₁-C₄ alkyl substituted with one or more groups each independently selected from —OH, C₁-C₂ alkoxy, C₁-C₄ haloalkyl (e.g., —CH₂F, —CHF₂, —CF₃, —CH₂CH₂F, —CH₂CHF₂, —CH₂CF₃), and C₁-C₄ alkoxy;
and all other variables are as defined in any one of the hundredth-and-fifth through two-hundred-and-tenth embodiments. In some embodiments of the two-hundred-and-forth embodiment, Z is a substituted or an unsubstituted 5- to 10-membered heteroaryl (e.g., a substituted or an unsubstituted 5- to 10-membered heteroaryl, a substituted or an unsubstituted 5- to 8-membered heteroaryl, a substituted or an unsubstituted 5- to 6-membered heteroaryl and a substituted or an unsubstituted 5- to 6-membered monocyclic heteroaryl such as a substituted or an unsubstituted pyrrolyl or a substituted or an unsubstituted pyridinyl), wherein the substituted heteroaryl is substituted with C₁-C₄ alkyl (e.g., methyl).

In a two-hundred-and-fifth embodiment, in a compound represented by Formula (I′), (I′a), (I′b), (I′c), (I′d), (II′), (II′-1), (III′a), (III′b), (III′c), (III′d), (II′a-1), (III′b-1), (III′c-1), (III′d-1), (IV′a), (IV′a-1), (V′a), or (V′a-1), or a pharmaceutically acceptable salt thereof, and unless specifically defined differently:
any C₁-C₆ alkyl includes C₁-C₆ alkyl, C₁-C₄ alkyl, C₁-C₃ alkyl, C₁-C₂ alkyl, methyl, ethyl, etc.;
any C₁-C₄ alkyl includes C₁-C₄ alkyl, C₁-C₃ alkyl, C₁-C₂ alkyl, methyl, ethyl, etc.;
any C₁-C₃ alkyl includes C₁-C₃ alkyl, C₁-C₂ alkyl, methyl, ethyl, etc.;
any C₁-C₂ alkyl includes C₁-C₂ alkyl, methyl, ethyl, etc.;
any C₂-C₆ alkenyl includes C₂-C₆ alkenyl, C₂-C₄ alkenyl, C₂-C₃ alkenyl, etc.;
any C₂-C₄ alkenyl includes C₂-C₄ alkenyl, C₂-C₃ alkenyl, etc.;
any C₂-C₆ alkynyl includes C₂-C₆ alkynyl, C₂-C₄ alkynyl, C₂-C₃ alkynyl, etc.;
any C₂-C₄ alkynyl includes C₂-C₄ alkynyl, C₂-C₃ alkynyl, etc.;
any C₁-C₆ alkoxy includes C₁-C₆ alkoxy, C₁-C₄ alkoxy, C₁-C₃ alkoxy, C₁-C₂ alkoxy, methoxy, ethoxy, etc.;
any C₁-C₄ alkoxy includes C₁-C₄ alkoxy, C₁-C₃ alkoxy, C₁-C₂ alkoxy, methoxy, ethoxy, etc.;
any C₁-C₃ alkoxy includes C₁-C₃ alkoxy, C₁-C₂ alkoxy, methoxy, ethoxy, etc.;
any C₁-C₂ alkoxy includes C₁-C₂ alkoxy, methoxy, ethoxy, etc.;
any C₁-C₆ heteroalkyl (e.g., —(CH₂)ₘO(CH₂)ₙCH₃, —(CH₂)ₘNH(CH₂)ₙCH₃, —(CH₂)ₘS(CH₂)ₙCH₃, where m and n are each an integer) includes C₁-C₆ heteroalkyl, C₁-C₄ heteroalkyl, C₁-C₃ heteroalkyl, C₁-C₂ heteroalkyl, etc.;
any C₁-C₄ heteroalkyl includes C₁-C₄ heteroalkyl, C₁-C₃ heteroalkyl, C₁-C₂ heteroalkyl, etc.;
any C₁-C₃ heteroalkyl includes C₁-C₃ heteroalkyl, C₁-C₂ heteroalkyl, etc.;
any C₃-C₆ cycloalkyl includes C₃-C₆ cycloalkyl, C₄-C₆ cycloalkyl, C₃-C₅ cycloalkyl, C₃-C₄ cycloalkyl, cyclopropyl, cyclobutyl, cyclopentyl, cyclohexyl, etc.;
any C₄-C₆ cycloalkyl includes C₄-C₆ cycloalkyl, cyclobutyl, cyclopentyl, cyclohexyl, etc.;
any C₃-C₅ cycloalkyl includes C₃-C₅ cycloalkyl, C₃-C₄ cycloalkyl, cyclopropyl, cyclobutyl, cyclopentyl, etc.;
any C₃-C₄ cycloalkyl includes cyclopropyl or cyclobutyl;
any 6- or 10-membered aryl includes 6- or 10-membered aryl, phenyl, etc.;
any 4- to 10-membered heterocyclyl includes 4- to 10-membered heterocyclyl, 4- to 9-membered heterocyclyl, 4- to 8-membered heterocyclyl, 5- to 10-membered heterocyclyl, 5- to 9-membered heterocyclyl, 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 4- to 9-membered heterocyclyl includes 4- to 9-membered heterocyclyl, 4- to 8-membered heterocyclyl, 5- to 9-membered heterocyclyl, 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 5- to 10-membered heterocyclyl includes 5- to 10-membered heterocyclyl, 4- to 9-membered heterocyclyl, 4- to 8-membered heterocyclyl, 5- to 9-membered heterocyclyl, 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 5- to 9-membered heterocyclyl includes 5- to 9-membered heterocyclyl, 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 5- to 8-membered heterocyclyl includes 5- to 8-membered heterocyclyl, 5- or 6-membered heterocyclyl, 5-membered heterocyclyl, 6-membered heterocyclyl, 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 7- to 9-membered heterocyclyl includes 7- to 9-membered heterocyclyl, 7- to 9-membered spiro heterocyclyl, 7- or 8-membered heterocyclyl, 7- or 8-membered spiro heterocyclyl, 7-membered spiro heterocyclyl, 8-membered spiro heterocyclyl, 9-membered spiro heterocyclyl, etc., each of which containing, for example, 1, 2, 3, or 4 nitrogen atoms;
any 7- or 8-membered heterocyclyl includes 7- or 8-membered heterocyclyl, 7- or 8-membered spiro heterocyclyl, 7-membered spiro heterocyclyl, 8-membered spiro heterocyclyl, etc., each of which containing, for example, 1, 2, 3, or 4 nitrogen atoms;
any 5- to 10-membered heteroaryl includes 5- to 10-membered heteroaryl, 5- to 8-membered heteroaryl, 5- or 6-membered heteroaryl, 5-membered heteroaryl, 6-heteroaryl, etc., each of which containing, for example, 1, 2, 3, or 4 nitrogen atoms;
any 5- to 8-membered heteroaryl includes 5- to 8-membered heteroaryl, 5- or 6-membered heteroaryl, 5-membered heteroaryl, 6-heteroaryl, etc., each of which containing, for example, 1, 2, 3, or 4 nitrogen atoms; and
any 5- or 6-membered heteroaryl includes 5- or 6-membered heteroaryl, 5-membered heteroaryl, 6-heteroaryl, each of which containing, for example, 1, 2, 3, or 4 nitrogen atoms; and all other variables are as defined in any one of the hundredth-and-fifth through two-hundred-and-tenth embodiments.

In a two-hundred-and-sixth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), (III′d-1), (IV′a), (IV′a-1), (V′a), or (V′a-1), or a pharmaceutically acceptable salt thereof, and unless specifically defined differently, any group including any of the groups described in the two-hundred-and-fifth embodiment that is substituted can be substituted with one or more groups selected from:
halogen (including e.g., F, Cl, Br, etc.);
—CN;
—OH;
C₁-C₆ alkyl (including e.g., C₁-C₆ alkyl, C₁-C₄ alkyl, C₁-C₂ alkyl, methyl, ethyl, etc.);
C₁-C₆ heteroalkyl (including e.g., C₁-C₆ heteroalkyl, C₁-C₄ heteroalkyl, C₁-C₂ alkyl, etc.);
C₁-C₆ haloalkyl (including e.g., C₁-C₆ haloalkyl, C₁-C₄ haloalkyl, C₁-C₂ haloalkyl, halomethyl, haloethyl, etc.);
C₁-C₆ alkyl (including e.g., C₁-C₆ alkyl, C₁-C₄ alkyl, C₁-C₂ alkyl, methyl, ethyl, etc.) substituted with C₁-C₆ alkoxy (including e.g., C₁-C₆ alkoxy, C₁-C₄ alkoxy, C₁-C₂ alkoxy, methoxy, ethoxy, etc.);
C₁-C₆ alkoxy (including e.g., C₁-C₆ alkoxy, C₁-C₄ alkoxy, C₁-C₂ alkoxy, methoxy, ethoxy, etc.);
C₁-C₆ haloalkoxy (including e.g., C₁-C₆ haloalkoxy, C₁-C₄ haloalkoxy, C₁-C₂ haloalkoxy, halomethoxy, haloethoxy, etc.); and
C₃-C₆ cycloalkyl (including e.g., C₃-C₆ cycloalkyl, C₄-C₆ cycloalkyl, C₃-C₅ cycloalkyl, C₃-C₄ cycloalkyl, cyclopropyl, cyclobutyl, cyclopentyl, cyclohexyl, etc.); and all other variables are as defined in any one of the hundredth-and-fifth through two-hundred-and-tenth embodiments.

In a two-hundred-and-seventh embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), (III′d-1), (IV′a), (IV′a-1), (V′a), or (V′a-1), or a pharmaceutically acceptable salt thereof, and unless specifically defined differently, any group including any of the groups described in the two-hundred-and-fifth embodiment that is substituted can be substituted with one or more groups selected from:
halogen (including e.g., F, Cl, Br, etc.);
—CN;
—OH;
C₁-C₆ alkyl (including e.g., C₁-C₆ alkyl, C₁-C₄ alkyl, C₁-C₂ alkyl, methyl, ethyl, etc.);
C₁-C₆ haloalkyl (including e.g., C₁-C₆ haloalkyl, C₁-C₄ haloalkyl, C₁-C₂ haloalkyl, halomethyl, haloethyl, etc.);
C₁-C₆ alkyl (including e.g., C₁-C₆ alkyl, C₁-C₄ alkyl, C₁-C₂ alkyl, methyl, ethyl, etc.) substituted with C₁-C₆ alkoxy (including e.g., C₁-C₆ alkoxy, C₁-C₄ alkoxy, C₁-C₂ alkoxy, methoxy, ethoxy, etc.); and
C₁-C₆ alkoxy (including e.g., C₁-C₆ alkoxy, C₁-C₄ alkoxy, C₁-C₂ alkoxy, methoxy, ethoxy, etc.);
and all other variables are as defined in any one of the hundredth-and-fifth through two-hundred-and-tenth embodiments.

In a two-hundred-and-eighth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), (III′d-1), (IV′a), (IV′a-1), (V′a), or (V′a-1), or a pharmaceutically acceptable salt thereof, and unless specifically defined differently, any group including any of the groups described in the two-hundred-and-fifth embodiment that is substituted can be substituted with one or more groups selected from:
halogen (including e.g., F, Cl, Br, etc.);
—CN;
—OH;
C₁-C₆ alkyl (including e.g., C₁-C₆ alkyl, C₁-C₄ alkyl, C₁-C₂ alkyl, methyl, ethyl, etc.);
C₁-C₆ haloalkyl (including e.g., C₁-C₆ haloalkyl, C₁-C₄ haloalkyl, C₁-C₂ haloalkyl, halomethyl, haloethyl, etc.); and
C₁-C₆ alkoxy (including e.g., C₁-C₆ alkoxy, C₁-C₄ alkoxy, C₁-C₂ alkoxy, methoxy, ethoxy, etc.);
and all other variables are as defined in any one of the hundredth-and-fifth through two-hundred-and-tenth embodiments.

In a two-hundred-and-nineth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), (III′d-1), (IV′a), (IV′a-1), (V′a), or (V′a-1), or a pharmaceutically acceptable salt thereof, and unless specifically defined differently, any substituted alkyl group, including any substituted C₁-C₆ alkyl, substituted C₁-C₄ alkyl, substituted C₁-C₃ alkyl, substituted C₁-C₂ alkyl, substituted methyl, substituted ethyl, etc. can be substituted with one or more deuteriums.

In a two-hundred-and-tenth embodiment, in a compound represented by Formula (I′), (I′a), (I′b), (I′c), (I′d), (II′), (II′-1), (III′a), (III′b), (III′c), (III′d), (III′a-1), (III′b-1), (III′c-1), (III′d-1), (IV′a), (IV′a-1), (V′a), or (V′a-1), or a pharmaceutically acceptable salt thereof, and unless specifically defined differently, all hydrogen atoms of any substituted alkyl group including substituted C₁-C₆ alkyl, substituted C₁-C₄ alkyl, substituted C₁-C₃ alkyl, substituted C₁-C₂ alkyl, substituted methyl, substituted ethyl, etc. are each substituted with deuterium.

Various embodiments provide a compound (e.g., a compound of Formula (I) or (I′)), wherein the compound has a structure selected from those of Compound Nos. 1-A to 76-C, as listed in the following Table 1, or a pharmaceutically acceptable salt thereof:

TABLE 1
Exemplary Compound Nos. 1-A to 76-C.

| No. | Structure | Structure | Structure |
|---|---|---|---|
| 1-A 1-B 1-C | 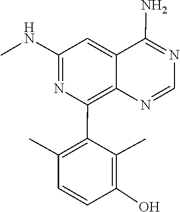 [SMILES: CNc1cc2c(N)ncnc2c(-c2c(C)ccc(O)c2C)n1] | 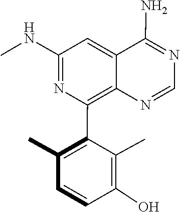 | 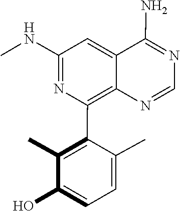 |
| | 1-A | 1-B | 1-C |
| 2-A 2-B 2-C | 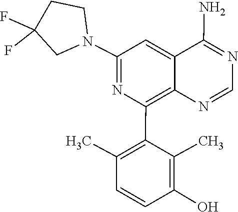 [SMILES: Cc1ccc(O)c(C)c1-c1nc(N2CCC(F)(F)C2)cc2c(N)ncnc12] | 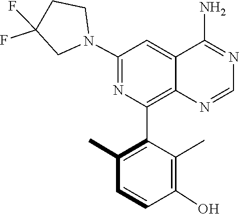 | 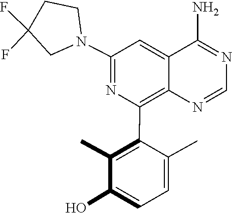 |
| | 2-A | 2-B | 2-C |
| 3-A 3-B 3-C | 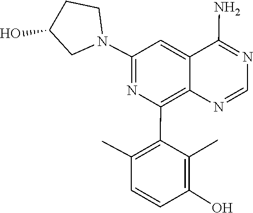 [SMILES: Cc1ccc(O)c(C)c1-c1nc(N2CC[C@@H](O)C2)cc2c(N)ncnc12] | 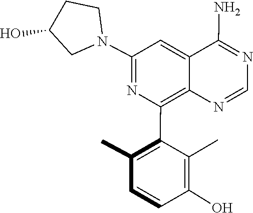 | 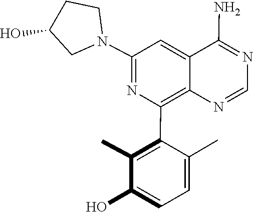 |
| | 3-A | 3-B | 3-C |
| 4-A 4-B 4-C | 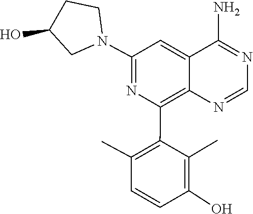 [SMILES: Cc1ccc(O)c(C)c1-c1nc(N2CC[C@H](O)C2)cc2c(N)ncnc12] | 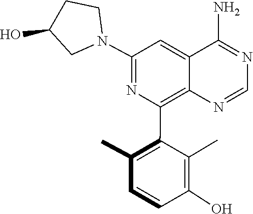 | 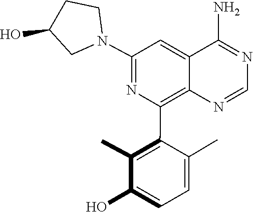 |
| | 4-A | 4-B | 4-C |
| 5-A 5-B 5-C | 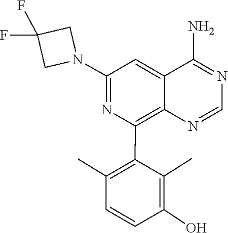 [SMILES: Cc1ccc(O)c(C)c1-c1nc(N2CC(F)(F)C2)cc2c(N)ncnc12] | 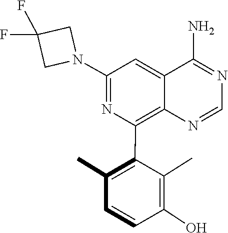 | 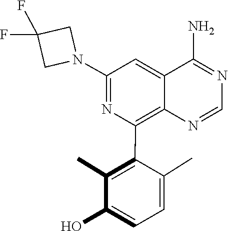 |
| | 5-A | 5-B | 5-C |
| 6-A 6-B 6-C | 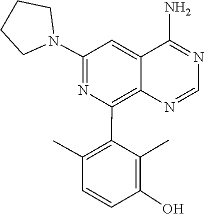 [SMILES: Cc1ccc(O)c(C)c1-c1nc(N2CCCC2)cc2c(N)ncnc12] | 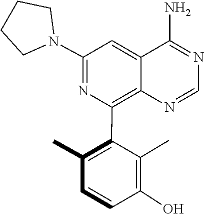 | 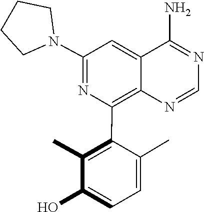 |
| | 6-A | 6-B | 6-C |
| 7-A 7-B 7-C | 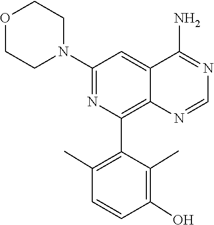 [SMILES: Cc1ccc(O)c(C)c1-c1nc(N2CCOCC2)cc2c(N)ncnc12] | 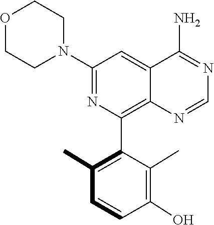 | 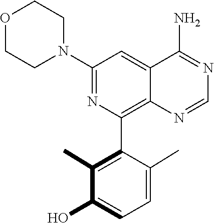 |
| | 7-A | 7-B | 7-C |
| 8-A 8-B 8-C | 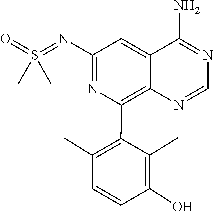 [SMILES: Cc1ccc(O)c(C)c1-c1nc(N=S(C)(C)=O)cc2c(N)ncnc12] | 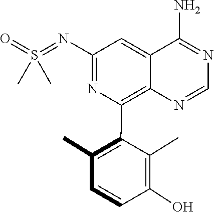 | 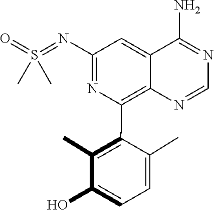 |
| | 8-A | 8-B | 8-C |
| 9-A 9-B 9-C | 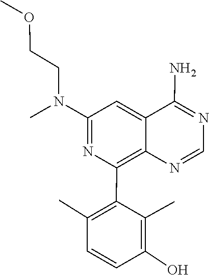 [SMILES: COCCN(C)c1cc2c(N)ncnc2c(-c2c(C)ccc(O)c2C)n1] | 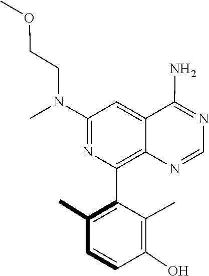 | 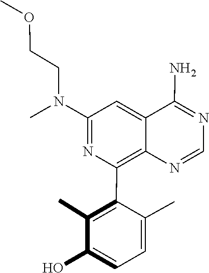 |
| | 9-A | 9-B | 9-C |
| 10-A 10-B 10-C | 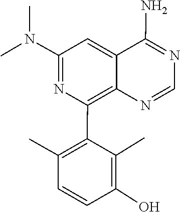 [SMILES: Cc1ccc(O)c(C)c1-c1nc(N(C)C)cc2c(N)ncnc12] | 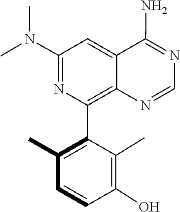 | 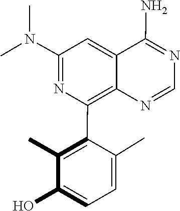 |
| | 10-A | 10-B | 10-C |
| 11-A 11-B 11-C | 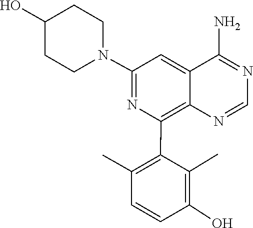 [SMILES: Cc1ccc(O)c(C)c1-c1nc(N2CCC(O)CC2)cc2c(N)ncnc12] | 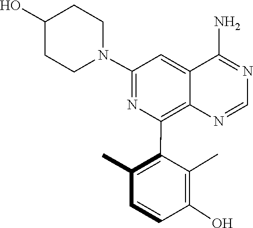 | 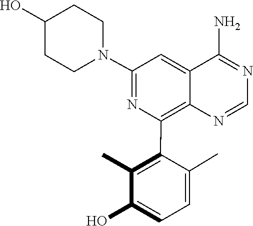 |
| | 11-A | 11-B | 11-C |
| 12-A 12-B 12-C | 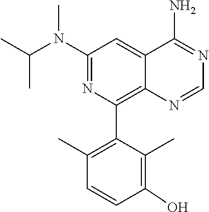 [SMILES: Cc1ccc(O)c(C)c1-c1nc(N(C)C(C)C)cc2c(N)ncnc12] | 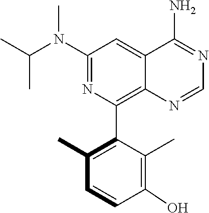 | 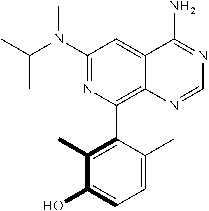 |
| | 12-A | 12-B | 12-C |
| 13-A 13-B 13-C | 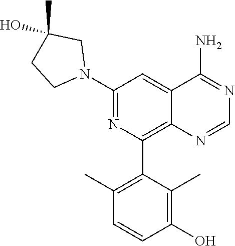 [SMILES: Cc1ccc(O)c(C)c1-c1nc(N2CC[C@](C)(O)C2)cc2c(N)ncnc12] | 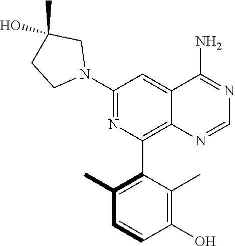 | 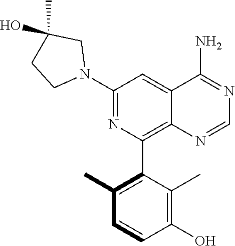 |
| | 13-A | 13-B | 13-C |
| 13-D 13-E 13-F | 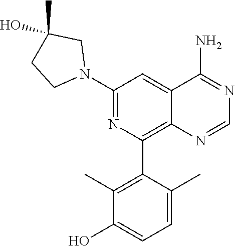 [SMILES: Cc1ccc(O)c(C)c1-c1nc(N2CC[C@](C)(O)C2)cc2c(N)ncnc12] | 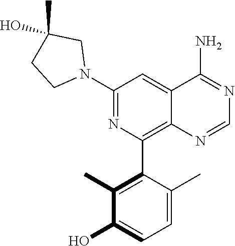 | 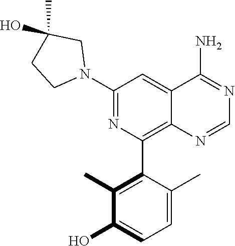 |
| | 13-D | 13-E | 13-F |
| 14-A 14-B 14-C | 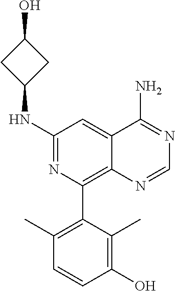 [SMILES: Cc1ccc(O)c(C)c1-c1nc(N[C@H]2C[C@@H](O)C2)cc2c(N)ncnc12] | 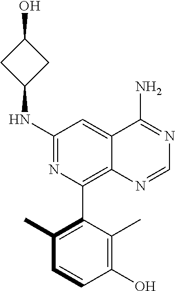 | 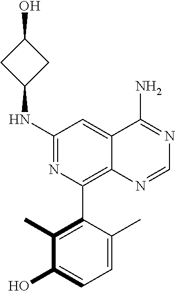 |
| | 14-A | 14-B | 14-C |
| 15-A 15-B 15-C | 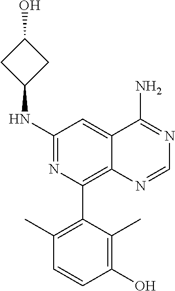 [SMILES: Cc1ccc(O)c(C)c1-c1nc(N[C@H]2C[C@H](O)C2)cc2c(N)ncnc12] | 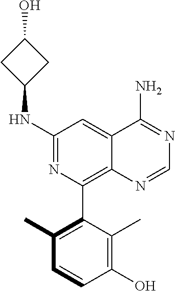 | 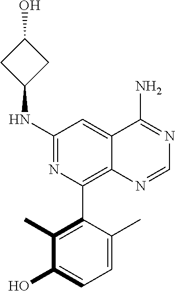 |
| | 15-A | 15-B | 15-C |
| 16-A 16-B 16-C | 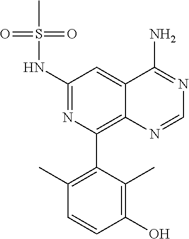 [SMILES: Cc1ccc(O)c(C)c1-c1nc(NS(C)(=O)=O)cc2c(N)ncnc12] | 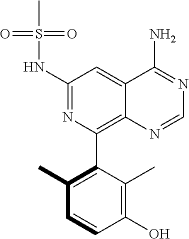 | 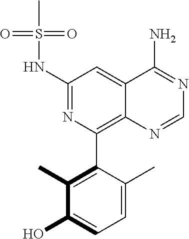 |
| | 16-A | 16-B | 16-C |
| 17-A 17-B 17-C | 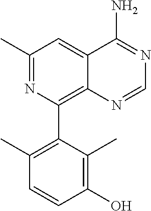 [SMILES: Cc1cc2c(N)ncnc2c(-c2c(C)ccc(O)c2C)n1] | 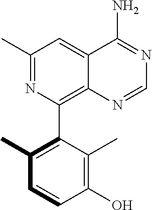 | 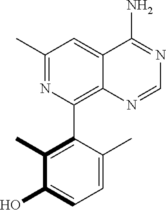 |
| | 17-A | 17-B | 17-C |
| 18-A 18-B 18-C | 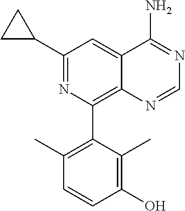 [SMILES: Cc1ccc(O)c(C)c1-c1nc(C2CC2)cc2c(N)ncnc12] | 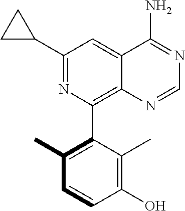 | 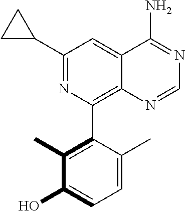 |
| | 18-A | 18-B | 18-C |
| 19-A 19-B 19-C | 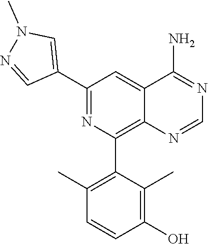 [SMILES: Cc1ccc(O)c(C)c1-c1nc(-c2cnn(C)c2)cc2c(N)ncnc12] | 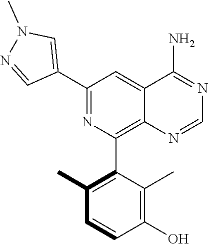 | 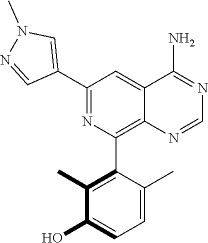 |
| | 19-A | 19-B | 19-C |
| 20-A 20-B 20-C | 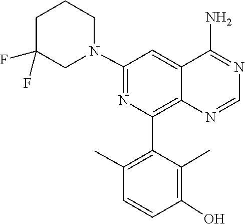 [SMILES: Cc1ccc(O)c(C)c1-c1nc(N2CCCC(F)(F)C2)cc2c(N)ncnc12] | 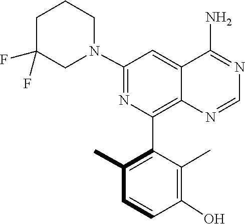 | 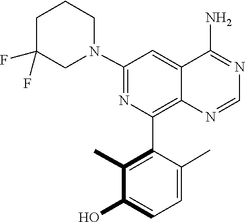 |
| | 20-A | 20-B | 20-C |
| 21-A 21-B 21-C | 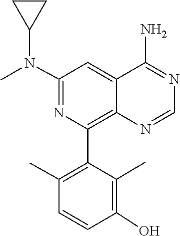 [SMILES: Cc1ccc(O)c(C)c1-c1nc(N(C)C2CC2)cc2c(N)ncnc12] | 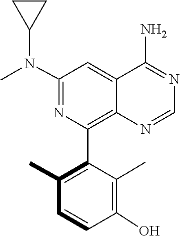 | 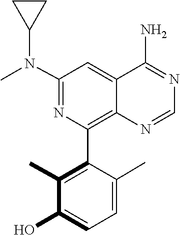 |
| | 21-A | 21-B | 21-C |
| 22-A 22-B 22-C | 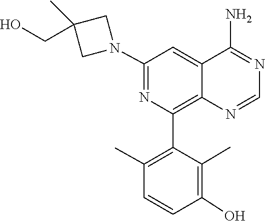 [SMILES: Cc1ccc(O)c(C)c1-c1nc(N2CC(C)(CO)C2)cc2c(N)ncnc12] | 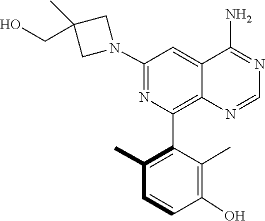 | 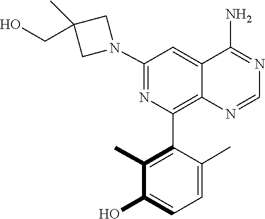 |
| | 22-A | 22-B | 22-C |
| 23-A 23-B 23-C | 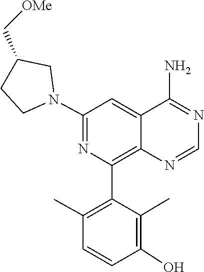 [SMILES: COC[C@H]1CCN(c2cc3c(N)ncnc3c(-c3c(C)ccc(O)c3C)n2)C1] | 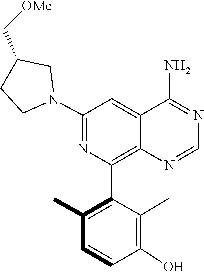 | 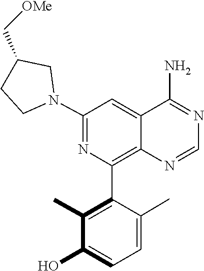 |
| | 23-A | 23-B | 23-C |
| 24-A 24-B 24-C | 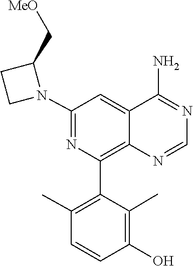 [SMILES: COC[C@@H]1CCN1c1cc2c(N)ncnc2c(-c2c(C)ccc(O)c2C)n1] | 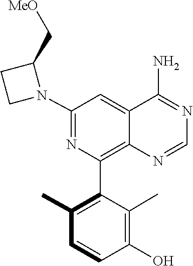 | 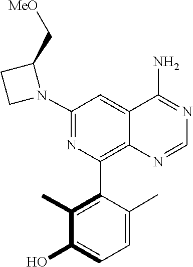 |
| | 24-A | 24-B | 24-C |
| 25-A 25-B 25-C | 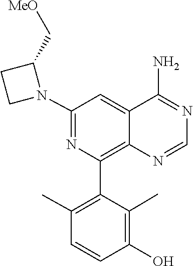 [SMILES: COC[C@H]1CCN1c1cc2c(N)ncnc2c(-c2c(C)ccc(O)c2C)n1] | 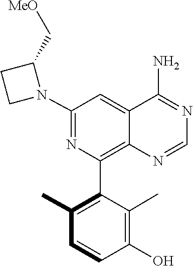 | 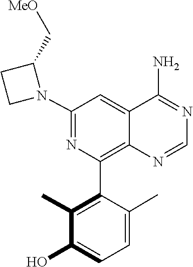 |
| | 25-A | 25-B | 25-C |
| 26-A 26-B 26-C | 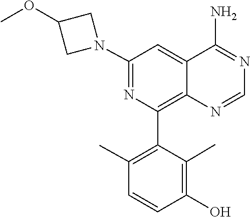 [SMILES: COC1CN(c2cc3c(N)ncnc3c(-c3c(C)ccc(O)c3C)n2)C1] | 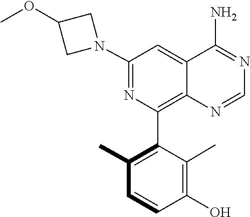 | 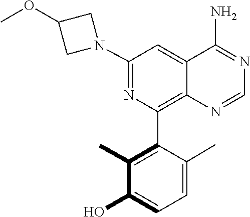 |
| | 26-A | 26-B | 26-C |
| 27-A 27-B 27-C | 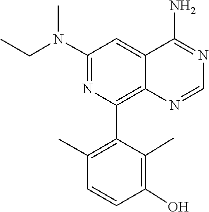 [SMILES: CCN(C)c1cc2c(N)ncnc2c(-c2c(C)ccc(O)c2C)n1] | 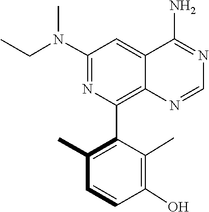 | 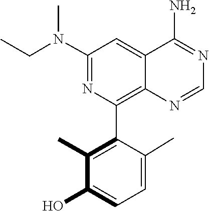 |
| | 27-A | 27-B | 27-C |
| 28-A 28-B 28-C | 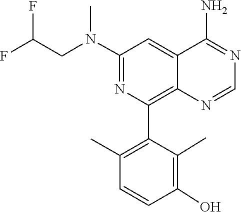 [SMILES: Cc1ccc(O)c(C)c1-c1nc(N(C)CC(F)F)cc2c(N)ncnc12] | 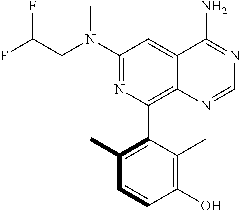 | 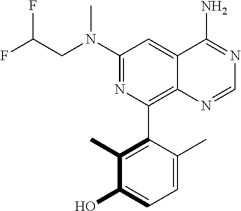 |
| | 28-A | 28-B | 28-C |
| 29-A 29-B 29-C | 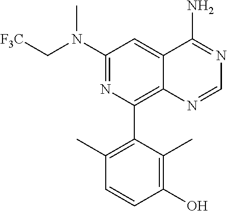 [SMILES: Cc1ccc(O)c(C)c1-c1nc(N(C)CC(F)(F)F)cc2c(N)ncnc12] | 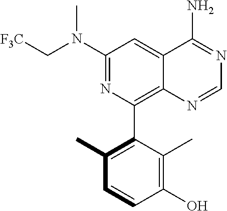 | 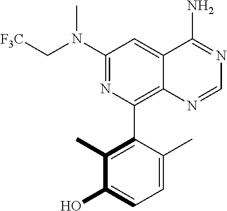 |
| | 29-A | 29-B | 29-C |
| 30-A 30-B 30-C | 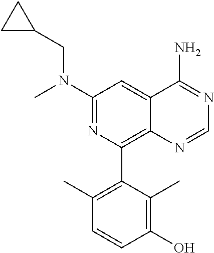 [SMILES: Cc1ccc(O)c(C)c1-c1nc(N(C)CC2CC2)cc2c(N)ncnc12] | 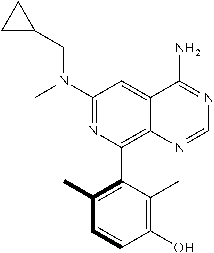 | 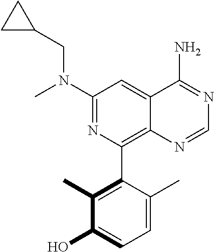 |
| | 30-A | 30-B | 30-C |
| 31-A 31-B 31-C | 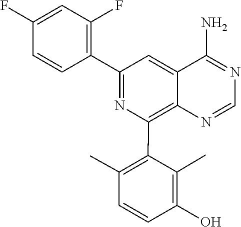 [SMILES: Cc1ccc(O)c(C)c1-c1nc(-c2ccc(F)cc2F)cc2c(N)ncnc12] | 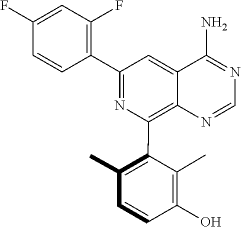 | 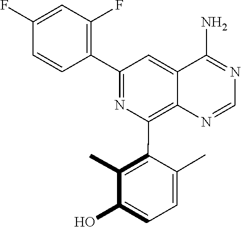 |
| | 31-A | 31-B | 31-C |
| 32-A 32-B 32-C | 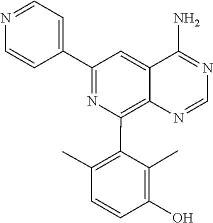 [SMILES: Cc1ccc(O)c(C)c1-c1nc(-c2ccncc2)cc2c(N)ncnc12] | 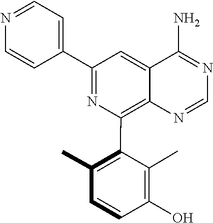 | 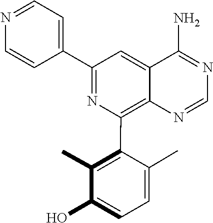 |
| | 32-A | 32-B | 32-C |
| 33-A 33-B 33-C | 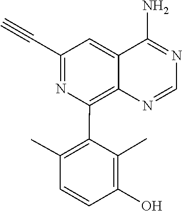 [SMILES: C#Cc1cc2c(N)ncnc2c(-c2c(C)ccc(O)c2C)n1] | 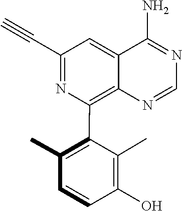 | 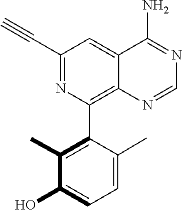 |
| | 33-A | 33-B | 33-C |
| 34-A 34-B 34-C | 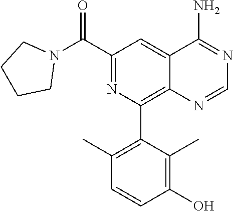 [SMILES: Cc1ccc(O)c(C)c1-c1nc(C(=O)N2CCCC2)cc2c(N)ncnc12] | 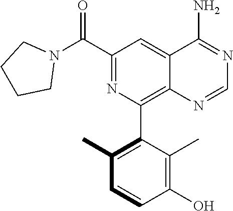 | 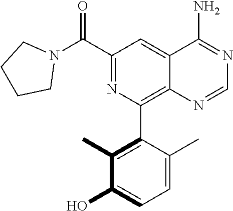 |
| | 34-A | 34-B | 34-C |
| 35-A 35-B 35-C | 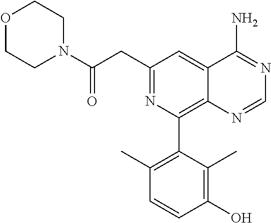 [SMILES: Cc1ccc(O)c(C)c1-c1nc(CC(=O)N2CCOCC2)cc2c(N)ncnc12] | 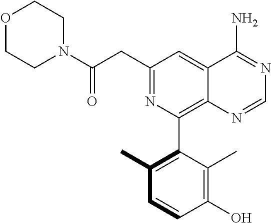 | 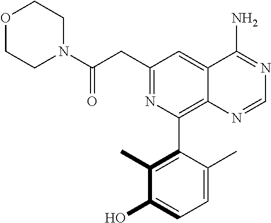 |
| | 35-A | 35-B | 35-C |
| 36-A 36-B 36-C | 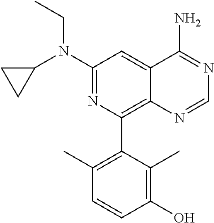 [SMILES: CCN(c1cc2c(N)ncnc2c(-c2c(C)ccc(O)c2C)n1)C1CC1] | 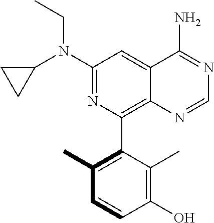 | 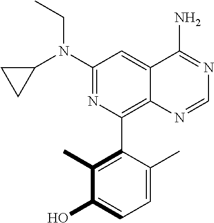 |
| | 36-A | 36-B | 36-C |
| 37-A 37-B 37-C | 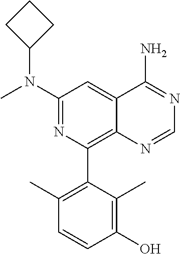 [SMILES: Cc1ccc(O)c(C)c1-c1nc(N(C)C2CCC2)cc2c(N)ncnc12] | 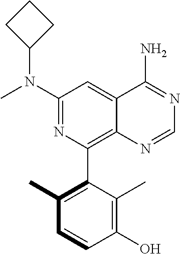 | 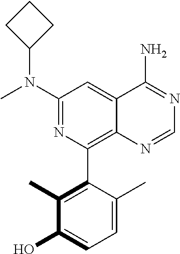 |
| | 37-A | 37-B | 37-C |
| 38-A 38-B 38-C | 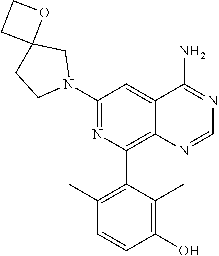 [SMILES: Cc1ccc(O)c(C)c1-c1nc(N2CCC3(CCO3)C2)cc2c(N)ncnc12] | 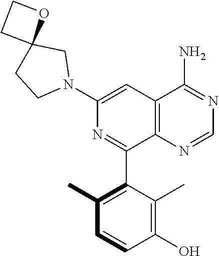 | 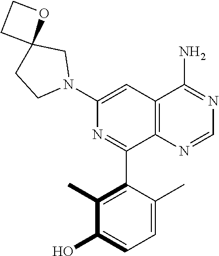 |
| | 38-A | 38-B | 38-C |
| 38-D 38-E | 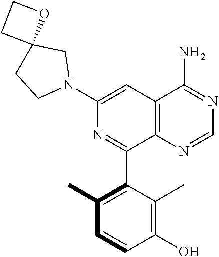 | 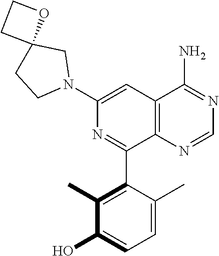 | |
| | 38-D | 38-E | |
| 39-A 39-B 39-C | 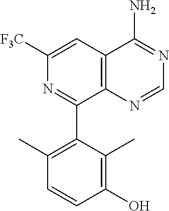 [SMILES: Cc1ccc(O)c(C)c1-c1nc(C(F)(F)F)cc2c(N)ncnc12] | 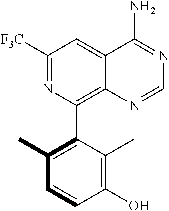 | 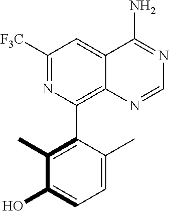 |
| | 39-A | 39-B | 39-C |
| 40-A 40-B 40-C | 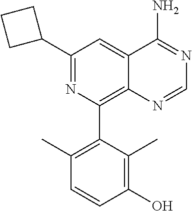 [SMILES: Cc1ccc(O)c(C)c1-c1nc(C2CCC2)cc2c(N)ncnc12] | 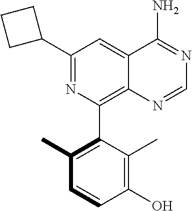 | 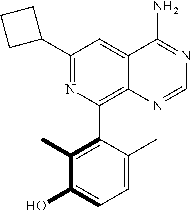 |
| | 40-A | 40-B | 40-C |
| 41-A 41-B 41-C | 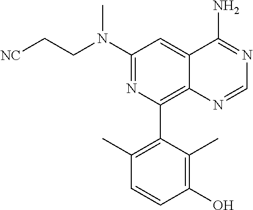 [SMILES: Cc1ccc(O)c(C)c1-c1nc(N(C)CCC#N)cc2c(N)ncnc12] | 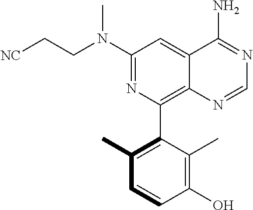 | 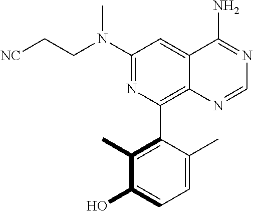 |
| | 41-A | 41-B | 41-C |
| 42-A 42-B 42-C | 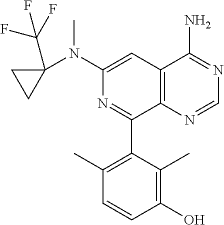 [SMILES: Cc1ccc(O)c(C)c1-c1nc(N(C)C2(C(F)(F)F)CC2)cc2c(N)ncnc12] | 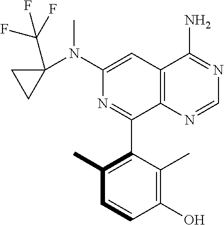 | 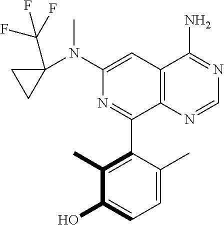 |
| | 42-A | 42-B | 42-C |
| 43-A 43-B 43-C | 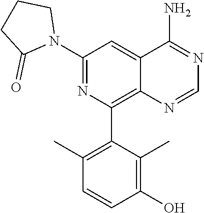 [SMILES: Cc1ccc(O)c(C)c1-c1nc(N2CCCC2=O)cc2c(N)ncnc12] | 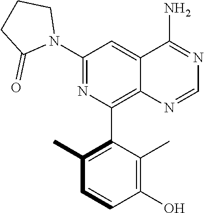 | 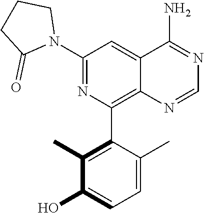 |
| | 43-A | 43-B | 43-C |
| 44-A 44-B 44-C | 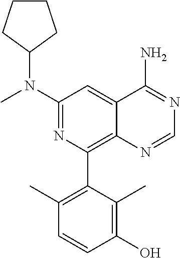 [SMILES: Cc1ccc(O)c(C)c1-c1nc(N(C)C2CCCC2)cc2c(N)ncnc12] | 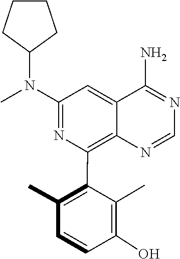 | 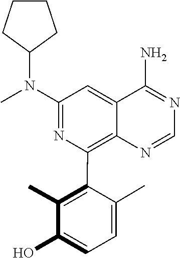 |
| | 44-A | 44-B | 44-C |
| 45-A 45-B 45-C | 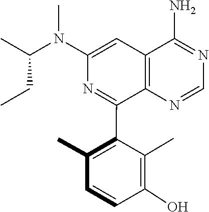 | 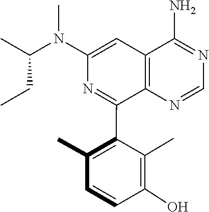 | 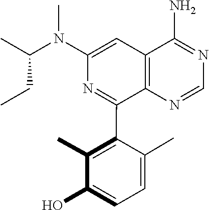 |
| | 45-A | 45-B | 45-C |
| 46-A 46-B 46-C | 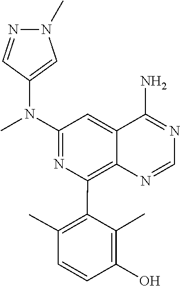 [SMILES: Cc1ccc(O)c(C)c1-c1nc(N(C)c2cnn(C)c2)cc2c(N)ncnc12] | 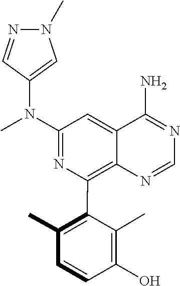 | 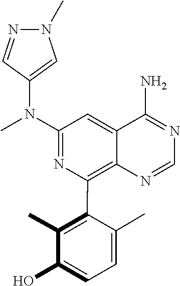 |
| | 46-A | 46-B | 46-C |
| 47-A 47-B 47-C | 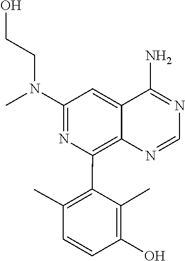 [SMILES: Cc1ccc(O)c(C)c1-c1nc(N(C)CCO)cc2c(N)ncnc12] | 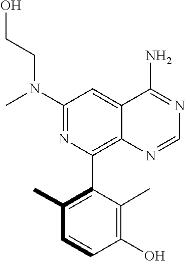 | 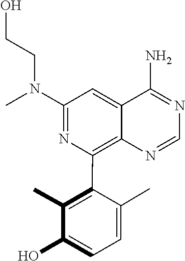 |
| | 47-A | 47-B | 47-C |
| 48-A 48-B 48-C | 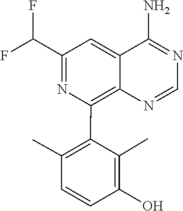 [SMILES: Cc1ccc(O)c(C)c1-c1nc(C(F)F)cc2c(N)ncnc12] | 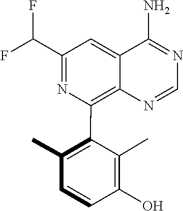 | 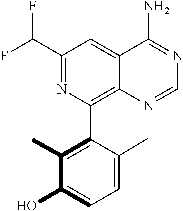 |
| | 48-A | 48-B | 48-C |
| 49-A 49-B 49-C | 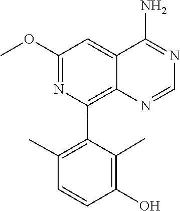 [SMILES: COc1cc2c(N)ncnc2c(-c2c(C)ccc(O)c2C)n1] | 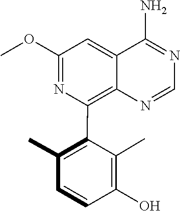 | 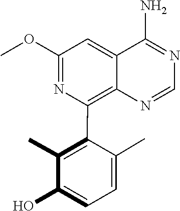 |
| | 49-A | 49-B | 49-C |
| 50-A 50-B 50-C | 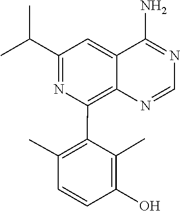 [SMILES: Cc1ccc(O)c(C)c1-c1nc(C(C)C)cc2c(N)ncnc12] | 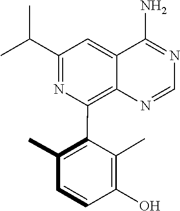 | 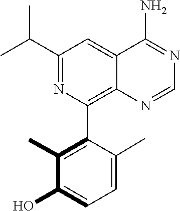 |
| | 50-A | 50-B | 50-C |
| 51-A 51-B 51-C | 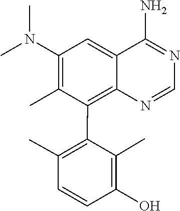 [SMILES: Cc1ccc(O)c(C)c1-c1c(C)c(N(C)C)cc2c(N)ncnc12] | 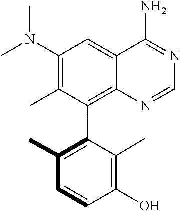 | 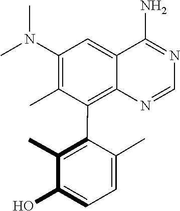 |
| | 51-A | 51-B | 51-C |
| 52-A 52-B 52-C | 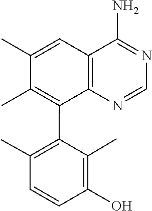 [SMILES: Cc1cc2c(N)ncnc2c(-c2c(C)ccc(O)c2C)c1C] | 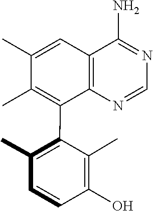 | 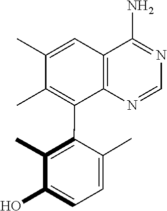 |
| | 52-A | 52-B | 52-C |
| 53-A 53-B 53-C | 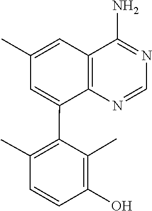 [SMILES: Cc1cc(-c2c(C)ccc(O)c2C)c2ncnc(N)c2c1] | 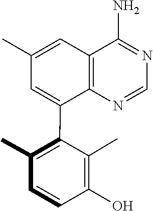 | 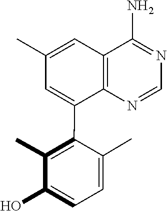 |
| | 53-A | 53-B | 53-C |
| 54-A 54-B 54-C | 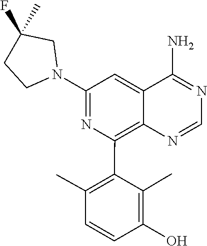 [SMILES: Cc1ccc(O)c(C)c1-c1nc(N2CC[C@@](C)(F)C2)cc2c(N)ncnc12] | 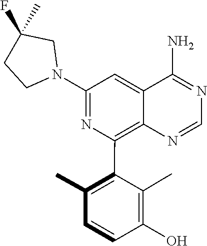 | 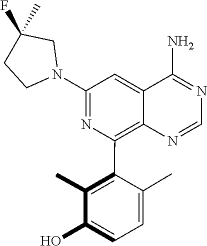 |
| | 54-A | 54-B | 54-C |
| 55-A 55-B 55-C | 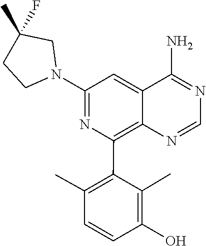 [SMILES: Cc1ccc(O)c(C)c1-c1nc(N2CC[C@](C)(F)C2)cc2c(N)ncnc12] | 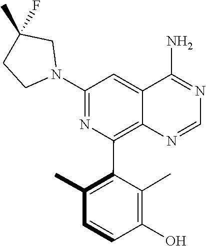 | 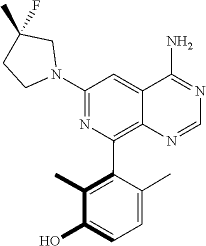 |
| | 55-A | 55-B | 55-C |
| 56-A 56-B 56-C | 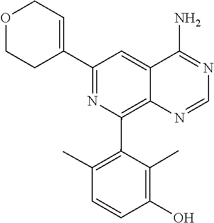 [SMILES: Cc1ccc(O)c(C)c1-c1nc(C2=CCOCC2)cc2c(N)ncnc12] | 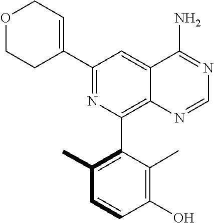 | 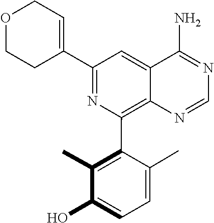 |
| | 56-A | 56-B | 56-C |
| 57-A 57-B 57-C | 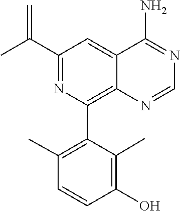 [SMILES: C=C(C)c1cc2c(N)ncnc2c(-c2c(C)ccc(O)c2C)n1] | 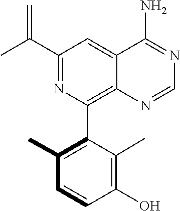 | 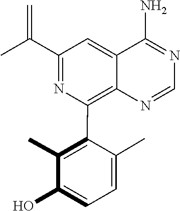 |
| | 57-A | 57-B | 57-C |
| 58-A 58-B 58-C | 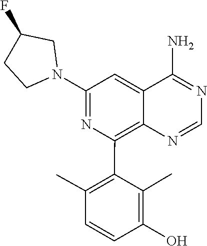 [SMILES: Cc1ccc(O)c(C)c1-c1nc(N2CC[C@@H](F)C2)cc2c(N)ncnc12] | 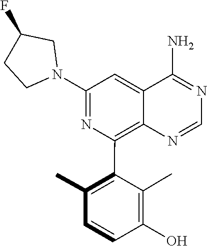 | 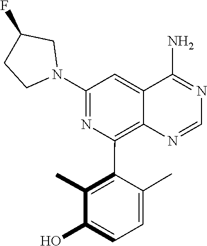 |
| | 58-A | 58-B | 58-C |
| 59-A 59-B 59-C | 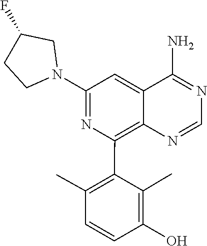 [SMILES: Cc1ccc(O)c(C)c1-c1nc(N2CC[C@H](F)C2)cc2c(N)ncnc12] | 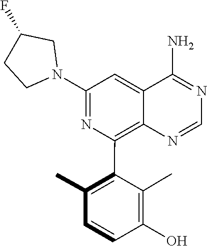 | 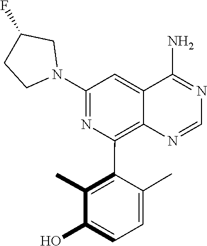 |
| | 59-A | 59-B | 59-C |
| 60-A 60-B 60-C | 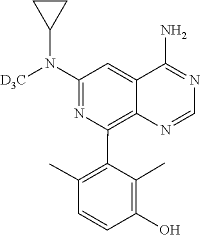 [SMILES: [2H]C([2H])([2H])N(c1cc2c(N)ncnc2c(-c2c(C)ccc(O)c2C)n1)C1CC1] | 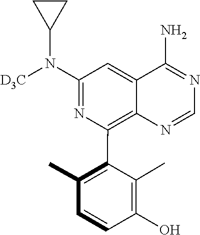 | 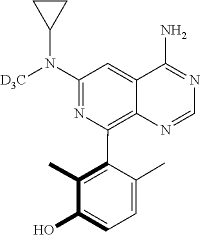 |
| | 60-A | 60-B | 60-C |
| 61-A 61-B 61-C | 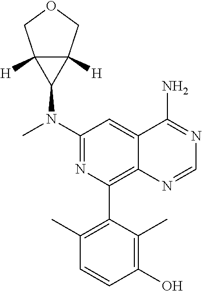 [SMILES: Cc1ccc(O)c(C)c1-c1nc(N(C)[C@H]2[C@@H]3COC[C@@H]32)cc2c(N)ncnc12] | 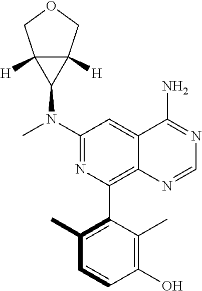 | 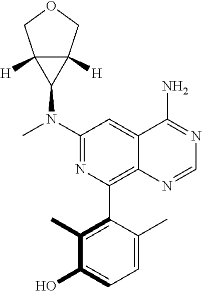 |
| | 61-A | 61-B | 61-C |
| 62-A 62-B 62-C | 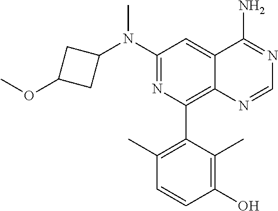 [SMILES: COC1CC(N(C)c2cc3c(N)ncnc3c(-c3c(C)ccc(O)c3C)n2)C1] | 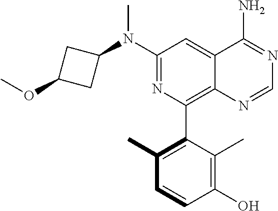 | 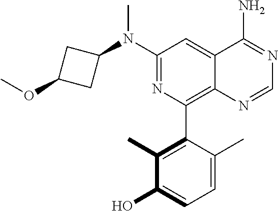 |
| | 62-A | 62-B | 62-C |
| 62-D 62-E | 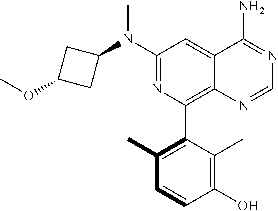 | 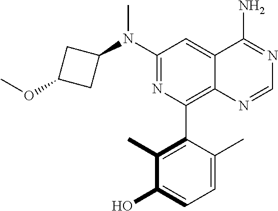 | |
| | 62-D | 62-E | |
| 63-A 63-B 63-C | 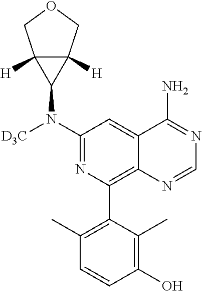 [SMILES: [2H]C([2H])([2H])N(c1cc2c(N)ncnc2c(-c2c(C)ccc(O)c2C)n1)[C@H]1[C@@H]2COC[C@@H]21] | 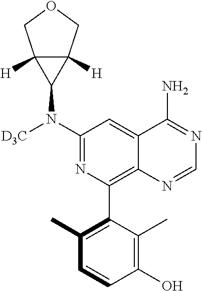 | 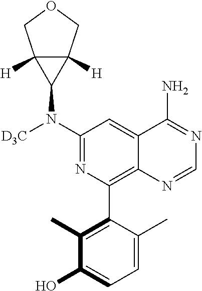 |
| | 63-A | 63-B | 63-C |
| 64-A 64-B 64-C | 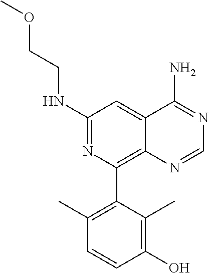 [SMILES: COCCNc1cc2c(N)ncnc2c(-c2c(C)ccc(O)c2C)n1] | 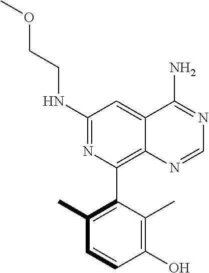 | 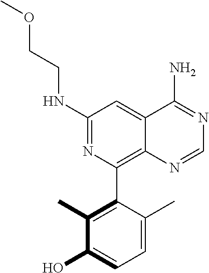 |
| | 64-A | 64-B | 64-C |
| 65-A 65-B 65-C | 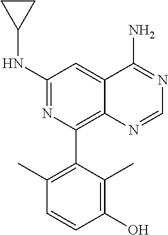 [SMILES: Cc1ccc(O)c(C)c1-c1nc(NC2CC2)cc2c(N)ncnc12] | 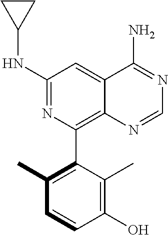 | 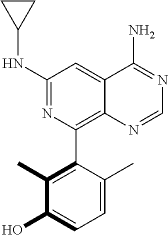 |
| | 65-A | 65-B | 65-C |
| 66-A 66-B 66-C | 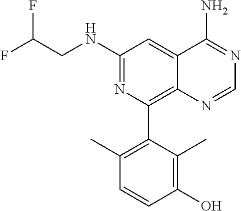 [SMILES: Cc1ccc(O)c(C)c1-c1nc(NCC(F)F)cc2c(N)ncnc12] | 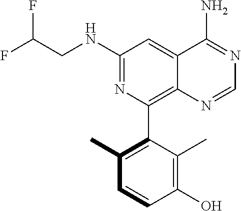 | 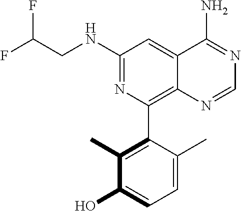 |
| | 66-A | 66-B | 66-C |
| 67-A 67-B 67-C | 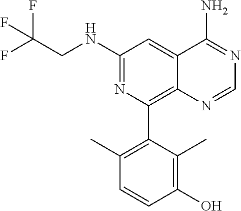 [SMILES: Cc1ccc(O)c(C)c1-c1nc(NCC(F)(F)F)cc2c(N)ncnc12] | 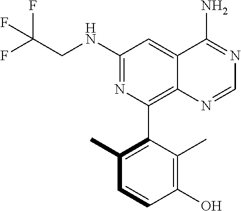 | 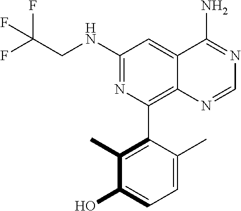 |
| | 67-A | 67-B | 67-C |
| 68-A 68-B 68-C | 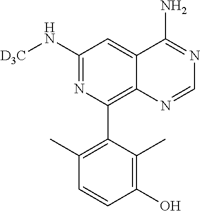 [SMILES: [2H]C([2H])([2H])Nc1cc2c(N)ncnc2c(-c2c(C)ccc(O)c2C)n1] | 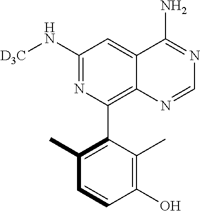 | 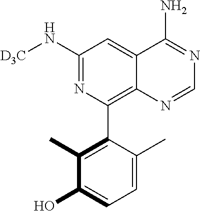 |
| | 68-A | 68-B | 68-C |
| 69-A 69-B 69-C | 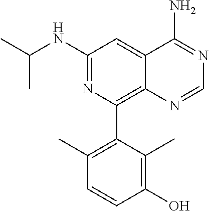 [SMILES: Cc1ccc(O)c(C)c1-c1nc(NC(C)C)cc2c(N)ncnc12] | 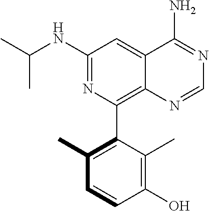 | 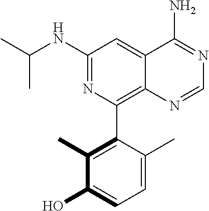 |
| | 69-A | 69-B | 69-C |
| 70-A 70-B 70-C | 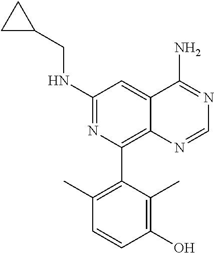 [SMILES: Cc1ccc(O)c(C)c1-c1nc(NCC2CC2)cc2c(N)ncnc12] | 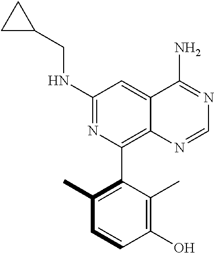 | 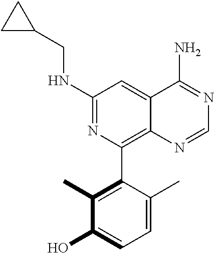 |
| | 70-A | 70-B | 70-C |
| 71-A 71-B 71-C | 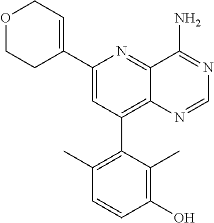 [SMILES: Cc1ccc(O)c(C)c1-c1cc(C2=CCOCC2)nc2c(N)ncnc12] | 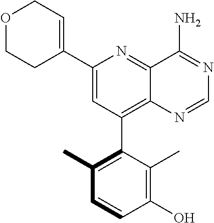 | 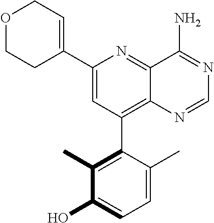 |
| | 71-A | 71-B | 71-C |
| 72-A 72-B 72-C | 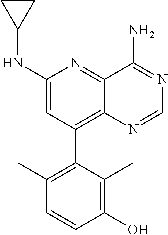 [SMILES: Cc1ccc(O)c(C)c1-c1cc(NC2CC2)nc2c(N)ncnc12] | 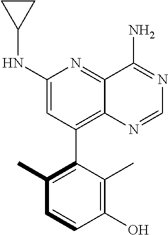 | 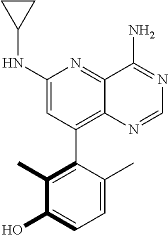 |
| | 72-A | 72-B | 72-C |
| 73-A 73-B 73-C | 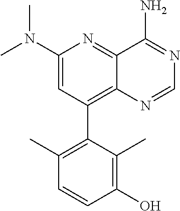 [SMILES: Cc1ccc(O)c(C)c1-c1cc(N(C)C)nc2c(N)ncnc12] | 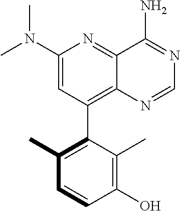 | 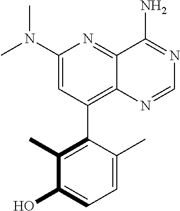 |
| | 73-A | 73-B | 73-C |
| 74-A 74-B 74-C | 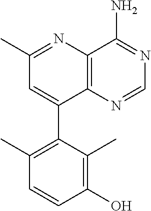 [SMILES: Cc1cc(-c2c(C)ccc(O)c2C)c2ncnc(N)c2n1] | 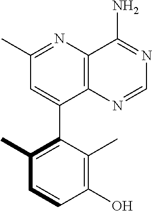 | 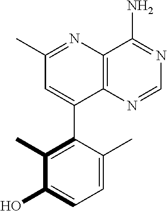 |
| | 74-A | 74-B | 74-C |
| 75-A 75-B 75-C | 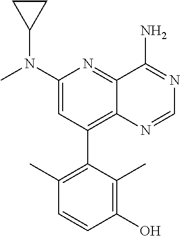 [SMILES: Cc1ccc(O)c(C)c1-c1cc(N(C)C2CC2)nc2c(N)ncnc12] | 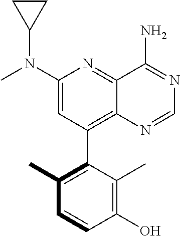 | 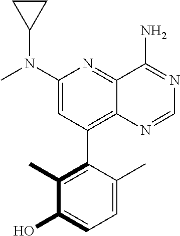 |
| | 75-A | 75-B | 75-C |
| 76-A 76-B 76-C | 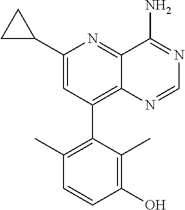 [SMILES: Cc1ccc(O)c(C)c1-c1cc(C2CC2)nc2c(N)ncnc12] | 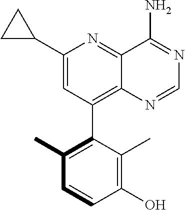 | 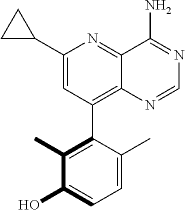 |
| | 76-A | 76-B | 76-C |

### Synthesis

Compounds of the Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or pharmaceutically acceptable salts of any of the foregoing, can be made in various ways by those skilled using known techniques as guided by the detailed teachings provided herein, including the Examples provided below. For example, in an embodiment, compounds of the Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, (including pharmaceutically acceptable salts of any of the foregoing) are prepared in accordance with the procedures illustrated in General Schemes 1 and 2, Intermediate Schemes 1-8 and Schemes 1-21 provided herein. Any preliminary reaction steps required to form starting compounds or other precursors can be carried out by those skilled in the art, for example by appropriate adjustment of the reagents and conditions described in the Examples. In General Schemes 1 and 2, the variables including Z, R^{A}, X¹, X², Y¹, Y², R^{B1}, R^{B2}, R^{C1}, R^{C4}, R^{C5}, R¹, and R² are as described elsewhere herein, taking into consideration the synthetic conversions involved as understood by those of skill in the art. Furthermore, in General Schemes 1 and 2, X is halogen.

### Pharmaceutical Compositions

Some embodiments described herein relate to a pharmaceutical composition, that can include an effective amount of one or more compounds described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) and a pharmaceutically acceptable carrier, diluent, excipient or combination thereof.

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

Some embodiments described herein relate to a method for ameliorating and/or treating a cancer described herein that can include administering an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) to a subject having a cancer described herein. Other embodiments described herein relate to the use of an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) in the manufacture of a medicament for ameliorating and/or treating a cancer described herein. Still other embodiments described herein relate to an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) for ameliorating and/or treating a cancer described herein.

Some embodiments described herein relate to a method for inhibiting replication of a malignant growth or a tumor that can include contacting the growth or the tumor with an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing), wherein the malignant growth or tumor is due to a cancer described herein. Other embodiments described herein relate to the use of an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) in the manufacture of a medicament for inhibiting replication of a malignant growth or a tumor, wherein the malignant growth or tumor is due to a cancer described herein. Still other embodiments described herein relate to an effective amount of a compound described herein (for example, a compound of Formula (I), (Ia), (Ib), (Ic), (II), (II-1), (IIIa), (IIIb), (IIIc), (IIId), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) for inhibiting replication of a malignant growth or a tumor, wherein the malignant growth or tumor is due to a cancer described herein.

Some embodiments described herein relate to a method for ameliorating or treating a cancer described herein that can include contacting a malignant growth or a tumor with an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) to a subject having a cancer described herein. Other embodiments described herein relate to the use of an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II—(I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) in the manufacture of a medicament for ameliorating or treating a cancer that can include contacting a malignant growth or a tumor, wherein the malignant growth or tumor is due to a cancer described herein. Still other embodiments described herein relate to an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) for ameliorating or treating a cancer that can include contacting a malignant growth or a tumor, wherein the malignant growth or tumor is due to a cancer described herein.

Some embodiments described herein relate to a method for inhibiting the activity of PKMYT1 (for example, inhibiting the activity in PKMYT1 CCNE1-overexpressing or CCNE1-amplified cells and/or decreasing the overexpression of PKMYT1 in cells) that can include providing an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) to a cancer cell from a cancer described herein. Other embodiments described herein relate to the use of an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt thereof) in the manufacture of a medicament for inhibiting the activity of PKMYT1 (for example, inhibiting the activity in PKMYT1 CCNE1-overexpressing or CCNE1-amplified cells and/or decreasing the overexpression of PKMYT1 in cells). Still other embodiments described herein relate to an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) for inhibiting the activity of PKMYT1 (for example, inhibiting the activity in PKMYT1 CCNE1-overexpressing or CCNE1-amplified cells and/or decreasing the overexpression of PKMYT1 in cells). Some embodiments described herein relate to a method for inhibiting the activity of PKMYT1 (for example, inhibiting the activity in PKMYT1 CCNE1-overexpressing or CCNE1-amplified cells and/or decreasing the overexpression of PKMYT1 in cells) that can include providing an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) to a cancer cell from a cancer described herein. Other embodiments described herein relate to a method for inhibiting the activity of PKMYT1 (for example, inhibiting the activity in PKMYT1 CCNE1-overexpressing or CCNE1-amplified cells and/or decreasing the overexpression of PKMYT1 in cells) that can include contacting a cancer cell from a cancer described herein with an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing), and thereby inhibiting the activity of PKMYT1.

Some embodiments described herein relate to a method for ameliorating or treating a cancer described herein that can include inhibiting the activity of PKMYT1 (for example, inhibiting the activity in PKMYT1 CCNE1-overexpressing or CCNE1-amplified cells and/or decreasing the overexpression of PKMYT1 in cells) using an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing). Other embodiments described herein relate to the use of an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) in the manufacture of a medicament for ameliorating or treating a cancer described herein by inhibiting the activity of PKMYT1 (for example, inhibiting the activity in PKMYT1 CCNE1-overexpressing or CCNE1-amplified cells and/or decreasing the overexpression of PKMYT1 in cells). Still other embodiments described herein relate to an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′—1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II—(I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) for ameliorating or treating a cancer described herein by inhibiting the activity of PKMYT1 (for example, inhibiting the activity in PKMYT1 CCNE1-overexpressing or CCNE1-amplified cells and/or decreasing the overexpression of PKMYT1 in cells). Some embodiments described herein relate to a method for ameliorating or treating a cancer described herein that can include contacting a cancer cell with an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′—1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing), wherein the compound inhibits the activity of PKMYT1 (for example, inhibiting the activity in PKMYT1 CCNE1-overexpressing or CCNE1-amplified cells and/or decreasing the overexpression of PKMYT1 in cells).

Some embodiments disclosed herein relate to a method for inhibiting the activity of PKMYT1 that can include providing an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) to a subject having a cancer described herein or a cancer cell from a cancer described herein. Other embodiments disclosed herein relate to the use of an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing, or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) in the manufacture of a medicament for inhibiting the activity of PKMYT1. Still other embodiments disclosed herein relate to a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt thereof) for inhibiting the activity of PKMYT1.

Examples of suitable cancers include, but are not limited to: brain cancers, cervicocerebral cancers, esophageal cancers, thyroid cancers, small cell cancers, non-small cell cancers, breast cancers, lung cancers (for example non-small cell lung cancer and small cell lung cancer), stomach cancers, gallbladder/bile duct cancers, liver cancers, pancreatic cancers, colon cancers, rectal cancers, ovarian cancers, choriocarcinomas, uterus body cancers, uterocervical cancers, renal pelvis/ureter cancers, bladder cancers, prostate cancers, penis cancers, testicular cancers, fetal cancers, Wilms' cancer, skin cancers, malignant melanoma, neuroblastomas, osteosarcomas, Ewing's tumors, soft part sarcomas, acute leukemia, chronic lymphatic leukemias, chronic myelocytic leukemias, polycythemia vera, malignant lymphomas, multiple myeloma, Hodgkin's lymphomas, and non-Hodgkin's lymphomas.

As described herein, a cancer can become resistant to one or more anti-cancer agents. In some embodiments, a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) or a pharmaceutical composition that includes an effective amount of a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) can be used to treat and/or ameliorate a cancer that has become resistant to one or more anti-cancer agents (such as one or more PKMYT1 inhibitors). Examples of anti-cancer agents that a subject may have developed resistance to include, but are not limited to, PKMYT1 inhibitors (such as RP-6306 or lunresertib, including pharmaceutically acceptable salts of any of the foregoing). In some embodiments, the cancer that has become resistant to one or more anti-cancer agents can be a cancer described herein.

Several known PKMYT1 inhibitors can cause one or more undesirable side effects in the subject being treated. Examples of undesirable side effects include, but are not limited to, thrombocytopenia, neutropenia, anemia, diarrhea, vomiting, nausea, abdominal pain, and constipation. In some embodiments, a compound described herein (for example, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing) can decrease the number and/or severity of one or more side effects associated with a known PKMYT1 inhibitor. In some embodiments, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing, can result in a severity of a side effect (such as one of those described herein) that is 25% less than compared to the severity of the same side effect experienced by a subject receiving a known PKMYT1 inhibitor (such as RP-6306, 1H-Pyrrolo[2,3-b]pyridine-3-carboxamide, 2-amino-1-(3-hydroxy-2,6-dimethylphenyl)-5,6-dimethyl-, (1S) or (1S)-2-Amino-1-(3-hydroxy-2,6-dimethylphenyl)-5,6-dimethyl-1H-pyrrolo[2,3-b]pyridine-3-carboxamide (including pharmaceutically acceptable salts of any of the foregoing)). In some embodiments, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), or Compound Nos. 1-A to 76-C, or a pharmaceutically acceptable salt of any of the foregoing, results in a number of side effects that is 25% less than compared to the number of side effects experienced by a subject receiving a known PKMYT1 inhibitor (for example, RP-6306). In some embodiments, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing, results in a severity of a side effect (such as one of those described herein) that is less in the range of about 10% to about 30% compared to the severity of the same side effect experienced by a subject receiving a known PKMYT1 inhibitor (such as RP-6306). In some embodiments, a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing, results in a number of side effects that is in the range of about 10% to about 30% less than compared to the number of side effects experienced by a subject receiving a known PKMYT1 inhibitor (for example, RP-6306, or a pharmaceutically acceptable salt thereof).

The one or more compounds of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′—1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing, that can be used to treat, ameliorate and/or inhibit the growth of a cancer wherein inhibiting the activity of PKMYT1 is beneficial is provided in any of the embodiments described under the heading titled “Compounds” above.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical agent, which elicits the biological or medicinal response indicated. For example, a therapeutically effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. In the treatment of lung cancer (such as non-small cell lung cancer) a therapeutically effective amount is that amount that alleviates or eliminates cough, shortness of breath and/or pain. As another example, an effective amount, or a therapeutically effective amount of an PKMYT1 inhibitor and/or degrader is the amount which results in the reduction in PKMYT1 activity and/or phosphorylation (such as phosphorylation of CDC2). The reduction in PKMYT1 activity is known to those skilled in the art and can be determined by the analysis of PKMYT1 intrinsic kinase activity and downstream substrate phosphorylation.

The amount of the compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or a pharmaceutically acceptable salt of any of the foregoing, required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, which is the dosage levels necessary to achieve the desired result, can be accomplished by one skilled in the art using routine methods, for example, human clinical trials, in vivo studies and in vitro studies. For example, useful dosages of a compound of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C, or pharmaceutically acceptable salts of any of the foregoing, can be determined by comparing their in vitro activity, and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

The compounds of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C (including pharmaceutically acceptable salts of any of the foregoing) as described herein are prepared using General Schemes 1 and 2 below.

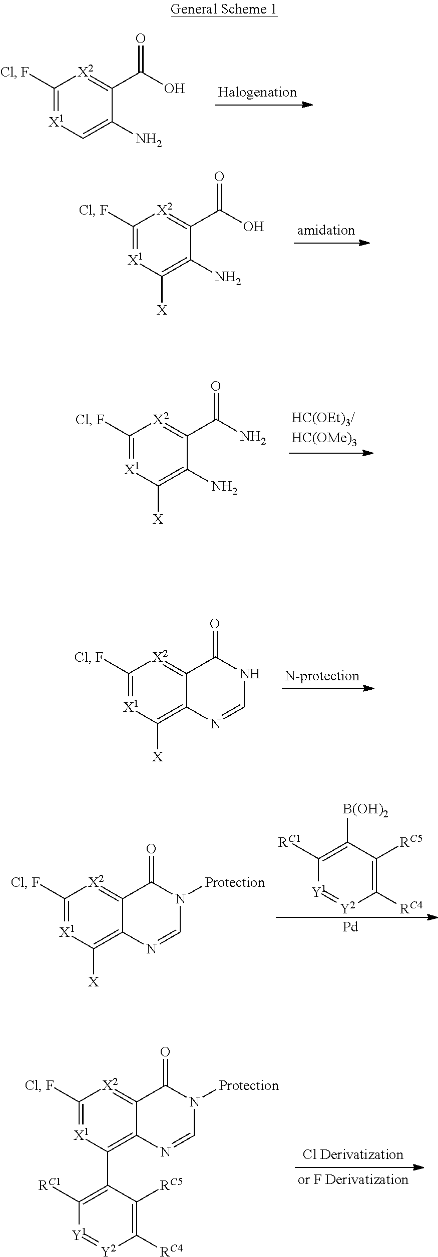

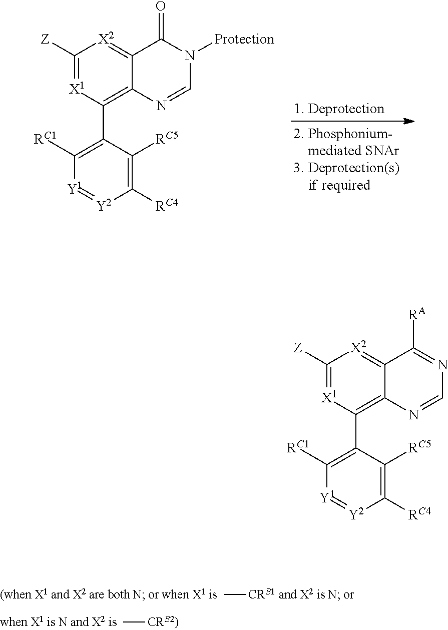

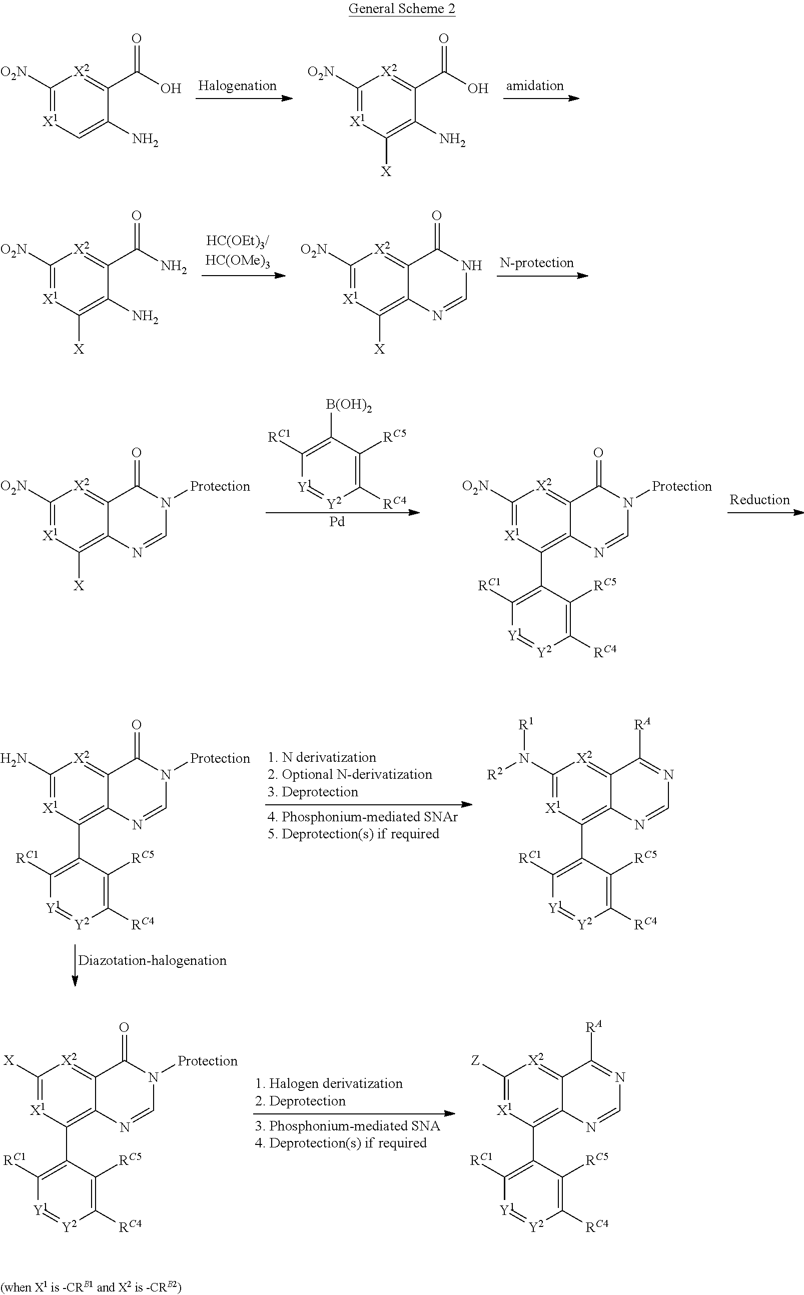

### Intermediate 1

### 6-Chloro-8-(3-methoxy-2,6-dimethylphenyl)-3-((2-(trimethylsilyl)ethoxy)methyl)pyrido[3,4-d]pyrimidin-4 (3H)-one (INT-1)

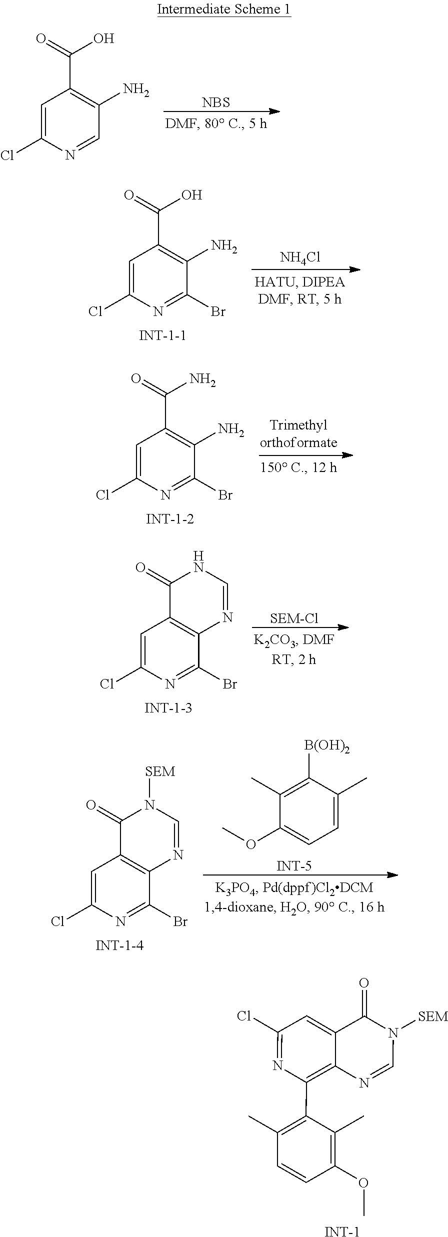

### 3-amino-2-bromo-6-chloroisonicotinic acid (INT-1-1)

To a solution of 5-amino-2-chloroisonicotinic acid (50.0 g, 289.74 mmol) in DMF (500 mL) was added NBS (61.89 g, 347.69 mmol.) at room temperature (rt) then heated at 80° C. for 5 h. After completion and cooling down to rt, the mixture was diluted with ice cold water (1 L). The resulting product was collected by filtration and dried under vacuum for 1 h then triturated in pentane (2×500 mL) to afford 3-amino-2-bromo-6-chloroisonicotinic acid (INT-1-1) (60 g, 82%). The material was used for the next step without further purification. MS (ESI) m/z: 253.12 [M+H]⁺.

### 3-amino-2-bromo-6-chloroisonicotinamide (INT-1-2)

To a solution of 3-amino-2-bromo-6-chloroisonicotinic acid (INT-1-1) (60.0 g, 238.60 mmol) and NH₄Cl (63.81 g, 1193.01 mmol) in DMF (600 mL) were added HATU (108.87 g, 286.32 mmol) and DIPEA (213.75 mL, 1193.01 mmol) at rt then stirred for 5 h. After completion, the reaction was quenched with cold water (3×3 L) and extracted with ethyl acetate (EA) (2×2 L). The combined organic layers were washed with brine (2 L), dried over MgSO₄, filtered, and concentrated to afford 3-amino-2-bromo-6-chloroisonicotinamide (INT-1-2) (55 g, 92%). The material was taken to the next step without further purification. MS (ESI) m/z: 250.1 [M]⁺.

### 8-bromo-6-chloropyrido[3,4-d]pyrimidin-4 (3H)-one (INT-1-3)

To a solution of 3-amino-2-bromo-6-chloroisonicotinamide (INT-1-2) (25.0 g, 99.81 mmol) in trimethyl orthoformate (750 mL) was added TsOH (0.17 g, 1.0 mmol) at rt. The mixture was stirred at 150° C. for 12 h. After completion, the mixture was concentrated to afford 8-bromo-6-chloropyrido[3,4-d]pyrimidin-4 (3H)-one (INT-1-3) (22 g, 84%). The material was taken to the next step without further purification. MS (ESI) m/z: 262.1 [M+2H]⁺.

### 8-bromo-6-chloro-3-((2-(trimethylsilyl)ethoxy)methyl)pyrido[3,4-d]pyrimidin-4 (3H)-one (INT-1-4)

To a solution of 8-bromo-6-chloropyrido[3,4-d]pyrimidin-4 (3H)-one (INT-1-3) (12.0 g, 46.07 mmol) in DMF (120 mL) was added K₂CO₃ (19.10 g, 138.21 mmol). The mixture was stirred at rt for 15 min then SEM-Cl (24.46 mL, 138.21 mmol) was added dropwise and stirred at rt for 2 h. After completion, the reaction was quenched with water (100 mL) and extracted with EtOAc (2×100 mL). The combined organic layers were washed with brine (150 mL), dried over MgSO₄, filtered, and concentrated. The residue was purified by column chromatography (SiO₂, EtOAc/petroleum ether (PET)) to afford 8-bromo-6-chloro-3-((2-(trimethylsilyl)ethoxy)methyl)pyrido[3,4-d]pyrimidin-4 (3H)-one (INT-1-4) (6.0 g, 33%). ¹H NMR (400 MHz, DMSO-d₆): δ ppm 8.66 (s, 1H), 8.09 (s, 1H), 5.37 (s, 2H), 3.64 (t, J=8.2 Hz, 2H), 0.89 (t, J=8.2 Hz, 2H), −0.03 (s, 9H). MS (ESI) m/z: 392.2 [M+2H]⁺.

### 6-chloro-8-(3-methoxy-2,6-dimethylphenyl)-3-((2-(trimethylsilyl)ethoxy)methyl)pyrido[3,4-d]pyrimidin-4 (3H)-one (INT-1)

To a stirred solution of 8-bromo-6-chloro-3-((2-(trimethylsilyl)ethoxy)methyl)pyrido[3,4-d]pyrimidin-4 (3H)-one (INT-1-4) (1.0 g, 2.56 mmol) and (3-methoxy-2,6-dimethylphenyl)boronic acid (INT-3) (0.46 g, 2.56 mmol) in 1,4-dioxane:water (3:1, 40 mL) was added K₃PO₄ (1.63 g, 7.68 mmol). The mixture was degassed with N₂ for 5 min followed by addition of Pd(dppf)Cl₂·CH₂Cl₂ (0.21 g, 0.26 mmol) then degassed again for 3 min and heated at 90° C. for 16 h. After completion, water (50 mL) was added and extracted with EtOAc (3×50 mL). The combined organic layers were washed with brine (50 mL), dried over Na₂SO₄, filtered, and concentrated. The residue was purified by column chromatography (SiO₂, 20% EtOAc/PE) to afford 6-chloro-8-(3-methoxy-2,6-dimethylphenyl)-3-((2-(trimethylsilyl)ethoxy)methyl)pyrido[3,4-d]pyrimidin-4 (3H)-one (INT-1) (0.70 g, 61%). MS (ESI) m/z: 446.4 [M+H]⁺.

### Intermediate 2

### 6-Chloro-8-(3-methoxy-2,6-dimethylphenyl)pyrido[3,4-d]pyrimidin-4-amine (INT-2)

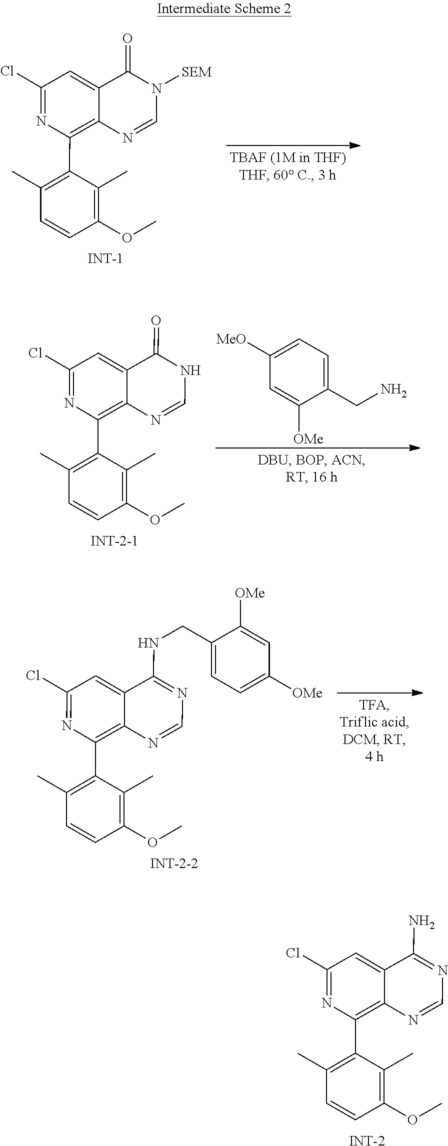

### 6-chloro-8-(3-methoxy-2,6-dimethylphenyl)pyrido[3,4-d]pyrimidin-4 (3H)-one (INT-2-1)

To a stirred solution of 6-chloro-8-(3-methoxy-2,6-dimethylphenyl)-3-((2-(trimethylsilyl)ethoxy)methyl)pyrido[3,4-d]pyrimidin-4 (3H)-one (INT-1) (6.0 g, 13.45 mmol) in THF (60 mL) was added TBAF (1M in THF, 25.56 mL, 40.36 mmol) at rt. The mixture was stirred at 60° C. for 3 h. After completion, the reaction was diluted with water (100 mL) and extracted with EtOAc (2×200 mL). The organic layers were separated, dried over MgSO₄, filtered, and concentrated. The residue was purified by column chromatography (SiO₂, EtOAc/PET) to afford 6-chloro-8-(3-methoxy-2,6-dimethylphenyl)pyrido[3,4-d]pyrimidin-4 (3H)-one (INT-2-1) (4.2 g, 96%). MS (ESI) m/z: 316.5 [M+H]⁺.

### 6-chloro-N-(2,4-dimethoxybenzyl)-8-(3-methoxy-2,6-dimethylphenyl)pyrido[3,4-d]pyrimidin-4-amine (INT-2-2)

To a stirred solution of 6-chloro-8-(3-methoxy-2,6-dimethylphenyl)pyrido[3,4-d]pyrimidin-4 (3H)-one (INT-2-1) (4.20 g, 13.30 mmol) and (2,4-dimethoxyphenyl)methanamine (4.45 g, 26.60 mmol) in ACN (42 mL) were added BOP (8.83 g, 19.95 mmol), Et₃N (2.64 mL, 26.60 mmol) at rt then stirred for 16 h. After completion, water (100 mL) was added then extracted with EtOAc (2×100 mL). The combined organic layers were washed with brine (100 mL), dried over Na₂SO₄, filtered, and concentrated. The residue was purified by column chromatography (SiO₂, EtOAc/PET) to afford 6-chloro-N-(2,4-dimethoxybenzyl)-8-(3-methoxy-2,6-dimethylphenyl)pyrido[3,4-d]pyrimidin-4-amine (INT-2-2) (4 g, 64%). MS (ESI) m/z: 465.7 [M+H]⁺.

### 6-chloro-8-(3-methoxy-2,6-dimethylphenyl)pyrido[3,4-d]pyrimidin-4-amine (INT-2)

To a stirred solution of 6-chloro-N-(2,4-dimethoxybenzyl)-8-(3-methoxy-2,6-dimethylphenyl)pyrido[3,4-d]pyrimidin-4-amine (INT-2-2) (4.0 g, 8.60 mmol) in DCM (40 mL) were added TFA (40 mL), TfOH (20 mL) at rt. The mixture was stirred at rt for 4 h. After completion, the reaction mixture was quenched with sat. NaHCO₃ (100 mL) and extracted with EtOAc (2×100 mL). The combined organic layers were washed with brine (100 mL), dried over MgSO₄, filtered, and concentrated. The residue was purified by prep-HPLC (C18, ACN/H₂O) to afford 6-chloro-8-(3-methoxy-2,6-dimethylphenyl)pyrido[3,4-d]pyrimidin-4-amine (INT-2) (1.2 g, 44%). ¹H NMR (400 MHz, DMSO-d₆): δ ppm 8.37 (d, J=6.8 Hz, 2H), 8.29 (br. s, 2H), 7.08 (d, J=8.4 Hz, 1H), 6.95 (d, J=8.4 Hz, 1H), 3.81 (s, 3H), 1.75 (s, 3H), 1.66 (s, 3H). MS (ESI) 315.2 [M+H]⁺.

### Intermediate 3

### 3-(4-Amino-6-fluoropyrido[3,4-d]pyrimidin-8-yl)-2,4-dimethylphenol (INT-3)

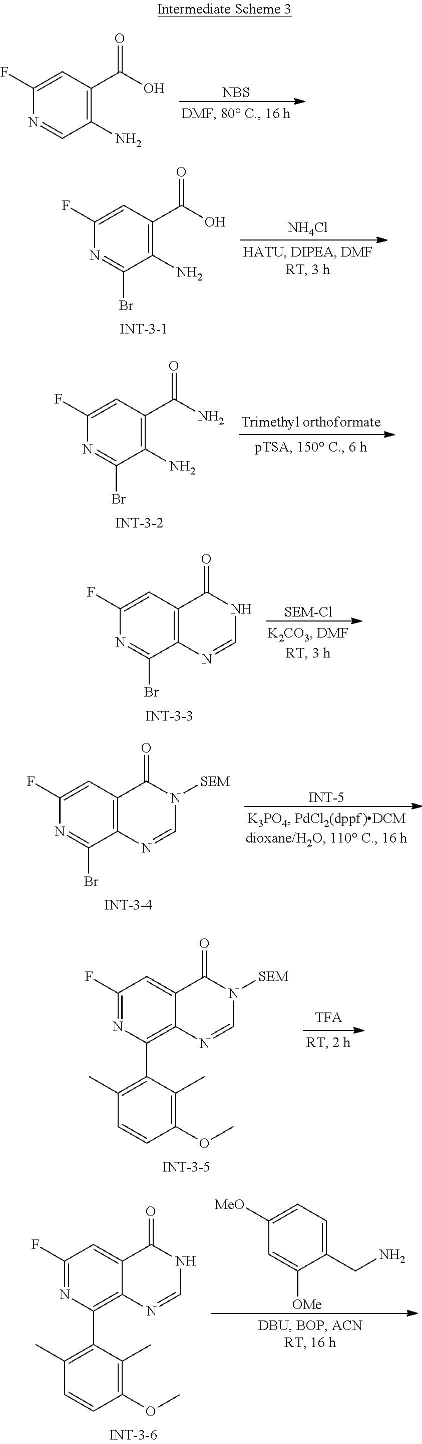

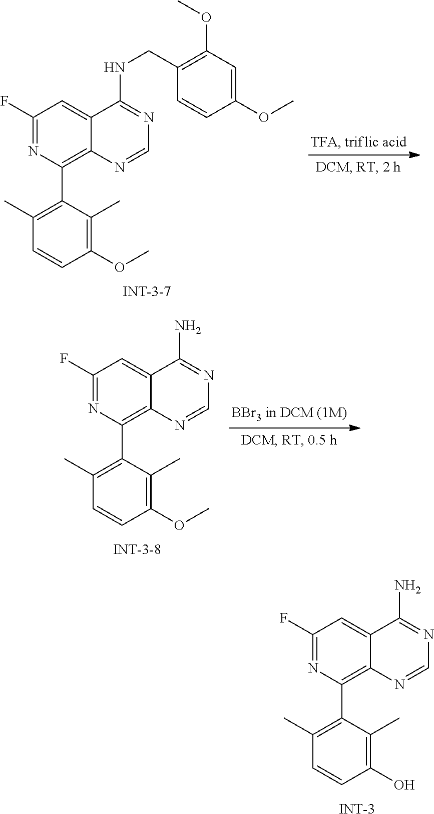

### 3-amino-2-bromo-6-fluoroisonicotinic acid (INT-3-1)

To a solution of 5-amino-2-fluoroisonicotinic acid (10.0 g, 64.06 mmol) in DMF (100 mL) was added NBS (13.68 g, 76.87 mmol) at rt. The mixture was heated at 80° C. for 16 h. After completion, water (500 mL) was added and extracted with EtOAc (2×500 mL). The combined organic layers were washed with brine (400 mL), dried over Na₂SO₄, filtered, and concentrated to afford 3-amino-2-bromo-6-fluoroisonicotinic acid (INT-3-1) (17.0 g, quantitative). The material was taken to the next step without further purification. MS (ESI) m/z: 237.1 [M+H]⁺.

### 3-amino-2-bromo-6-fluoroisonicotinamide (INT-3-2)

To a solution of 3-amino-2-bromo-6-fluoroisonicotinic acid (INT-3-1) (17.0 g, 72.65 mmol) and NH₄Cl (19.43 g, 363.25 mmol) in DMF (170 mL) were added HATU (41.44 g, 108.97 mmol) and DIPEA (65.08 mL, 363.24 mmol) at rt then stirred for 3 h. After completion, the reaction was quenched with water (600 mL) and extracted with EtOAc (2×600 mL). The combined organic layers were washed with brine (300 mL), dried over MgSO₄, filtered, and concentrated. The residue was purified by column chromatography (SiO₂, 50% EtOAc/PET) to afford 3-amino-2-bromo-6-fluoroisonicotinamide (INT-3-2) (12 g, quantitative). MS (ESI) m/z: 236.3 [M+H]⁺.

### 8-bromo-6-fluoropyrido[3,4-d]pyrimidin-4 (3H)-one (INT-3-3)

To a solution of 3-amino-2-bromo-6-fluoroisonicotinamide (INT-3-2) (12.0 g, 51.51 mmol) in trimethyl orthoformate (360 mL) was added p-TsOH (0.89 g, 5.15 mmol) at rt and heated in autoclave at 150° C. for 6 h. After completion, the mixture was concentrated to afford 8-bromo-6-fluoropyrido[3,4-d]pyrimidin-4 (3H)-one (INT-3-3) (11.0 g, quantitative). MS (ESI) m/z: 246.3 [M+H]⁺. The material was taken to the next step without further purification.

### 8-bromo-6-fluoro-3-((2-(trimethylsilyl)ethoxy)methyl)pyrido[3,4-d]pyrimidin-4 (3H)-one (INT-3-4)

To a solution of 8-bromo-6-fluoropyrido[3,4-d]pyrimidin-4 (3H)-one (INT-3-3) (11.0 g, 45.27 mmol) in DMF (110 mL) was added K₂CO₃ (18.77 g, 135.80 mmol). The mixture was stirred at rt for 15 min. then SEM-Cl (9.61 mL, 54.32 mmol) was added dropwise then stirred at rt for 3 h. After completion, the mixture was quenched with water (600 mL) and extracted with EtOAc (2×400 mL). The combined organic layers were washed with brine (200 mL), dried over MgSO₄, filtered, and concentrated. The residue was purified by column chromatography (SiO₂, 30% EtOAc/PET) to afford 8-bromo-6-fluoro-3-((2-(trimethylsilyl)ethoxy)methyl)pyrido[3,4-d]pyrimidin-4 (3H)-one (INT-3-4) (13.0 g, 77%). MS (ESI) m/z: 376.4 [M+H]⁺.

### 6-fluoro-8-(3-methoxy-2,6-dimethylphenyl)-3-((2-(trimethylsilyl)ethoxy)methyl)pyrido[3,4-d]pyrimidin-4 (3H)-one (INT-3-5)

To a solution of 8-bromo-6-chloro-3-((2-(trimethylsilyl)ethoxy)methyl)pyrido[3,4-d]pyrimidin-4 (3H)-one (INT-3-4) (5.0 g, 12.80 mmol), (3-methoxy-2,6-dimethylphenyl)boronic acid (2.34 g, 14.08 mmol) in 1,4-dioxane:water (4:1, 50 mL) was added K₃PO₄ (8.15 g, 38.39 mmol) and degassed with N₂ for 5 min. followed by addition of Pd(dppf)Cl₂-DCM (1.05 g, 1.28 mmol). The mixture was degassed again for 10 min. and heated at 110° C. for 16 h. After completion, water (200 mL) was added and extracted with EtOAc (2×200 mL). The combined organic layers were washed with brine (100 mL), dried over Na₂SO₄, filtered, and concentrated. The residue was purified by column chromatography (SiO₂, 25% EtOAc/PET) to afford 6-fluoro-8-(3-methoxy-2,6-dimethylphenyl)-3-((2-(trimethylsilyl)ethoxy)methyl)pyrido[3,4-d]pyrimidin-4 (3H)-one (INT-3-5) (3.0 g, 44%). MS (ESI) m/z: 430.7 [M+H]⁺.

### 6-fluoro-8-(3-methoxy-2,6-dimethylphenyl)pyrido[3,4-d]pyrimidin-4 (3H)-one (INT-3-6)

A solution of 6-fluoro-8-(3-methoxy-2,6-dimethylphenyl)-3-((2-(trimethylsilyl)ethoxy)methyl)pyrido[3,4-d]pyrimidin-4 (3H)-one (INT-3-5) (1.2 g, 2.79 mmol) in TFA (12 mL) was stirred at rt for 2 h. The reaction was quenched with sat. NaHCO₃ (20 mL) and extracted with EtOAc (2×100 mL). The combined organic layers were dried over Na₂SO₄, filtered, and concentrated. The residue was triturated with PET to afford 6-fluoro-8-(3-methoxy-2,6-dimethylphenyl)pyrido[3,4-d]pyrimidin-4 (3H)-one (INT-3-6) (0.83 g, 99%). This material was taken to the next step without further purification. MS (ESI) m/z: 300.5 [M+H]⁺.

### N-(2,4-dimethoxybenzyl)-6-fluoro-8-(3-methoxy-2,6-dimethylphenyl)pyrido[3,4-d]pyrimidin-4-amine (INT-3-7)

To a solution of 6-fluoro-8-(3-methoxy-2,6-dimethylphenyl)pyrido[3,4-d]pyrimidin-4 (3H)-one (INT-3-6) (0.83 g, 2.77 mmol) and (2,4-dimethoxyphenyl)methanamine (0.83 mL, 5.55 mmol) in ACN (8.3 mL) were added BOP (2.45 g, 5.55 mmol) and DBU (1.24 mL, 8.32 mmol) at rt then stirred for 16 h. The mixture was concentrated then diluted with water (50 mL) and extracted with EtOAc (100 mL). The organic layer was dried over Na₂SO₄, filtered, and concentrated. The residue was purified by column chromatography (SiO₂, 40% EtOAc/PET) to afford N-(2,4-dimethoxybenzyl)-6-fluoro-8-(3-methoxy-2,6-dimethylphenyl)pyrido[3,4-d]pyrimidin-4-amine (INT-3-7) (1.11 g, 89%). MS (ESI) m/z: 449.5 [M+H]⁺.

### 6-fluoro-8-(3-methoxy-2,6-dimethylphenyl)pyrido[3,4-d]pyrimidin-4-amine (INT-3-8)

To a solution of N-(2,4-dimethoxybenzyl)-6-fluoro-8-(3-methoxy-2,6-dimethylphenyl)pyrido[3,4-d]pyrimidin-4-amine (INT-3-7) (1.11 g, 2.48 mmol) in DCM (11.1 mL) were added TFA (3.79 mL, 49.499 mmol) and TfOH (2.18 mL, 24.75 mmol) at rt then stirred for 2 h. The reaction was quenched with sat. NaHCO₃ (50 mL) and extracted with EtOAc (2×100 mL). The combined organic layers were dried over Na₂SO₄, filtered, and concentrated to afford 6-fluoro-8-(3-methoxy-2,6-dimethylphenyl)pyrido[3,4-d]pyrimidin-4-amine (INT-3-8) (0.69 g, 93%). MS (ESI) m/z: 299.5 [M+H]⁺. The material was taken to the next step without further purification.

### 3-(4-amino-6-fluoropyrido[3,4-d]pyrimidin-8-yl)-2,4-dimethylphenol (INT-3)

To 6-fluoro-8-(3-methoxy-2,6-dimethylphenyl)pyrido[3,4-d]pyrimidin-4-amine (INT-3-8) (3.8 g, 12.74 mmol) in DCM (38 mL) was added BBr₃ (1M in DCM, 16 mL, 63.67 mmol) at 0° C. then stirred at rt for 1 h. The reaction was quenched with sat. NaHCO₃ (100 mL) and extracted with EtOAc (2×250 mL). The combined organic layers were dried over Na₂SO₄, filtered, and concentrated. The residue was purified by column chromatography (SiO₂, 60% EtOAc/PET) to afford 3-(4-amino-6-fluoropyrido[3,4-d]pyrimidin-8-yl)-2,4-dimethylphenol (INT-3) (1.50 g, 41%). ¹H NMR (400 MHz, DMSO-d₆): δ ppm 9.17 (s, 1H), 8.34 (s, 1H), 8.22 (br. s, 2H), 7.94 (d, J=2.0 Hz, 1H), 6.90 (d, J=8.0 Hz, 1H), 6.79 (d, J=8.0 Hz, 1H), 1.71 (s, 3H), 1.63 (s, 3H). MS (ESI) m/z: 285.5 [M+H]⁺.

### Intermediate 4

### tert-Butyl (8-(3-(benzyloxy)-2,6-dimethylphenyl)-6-chloropyrido[3,4-d]pyrimidin-4-yl)(2,4-dimethoxybenzyl)carbamate (INT-4)

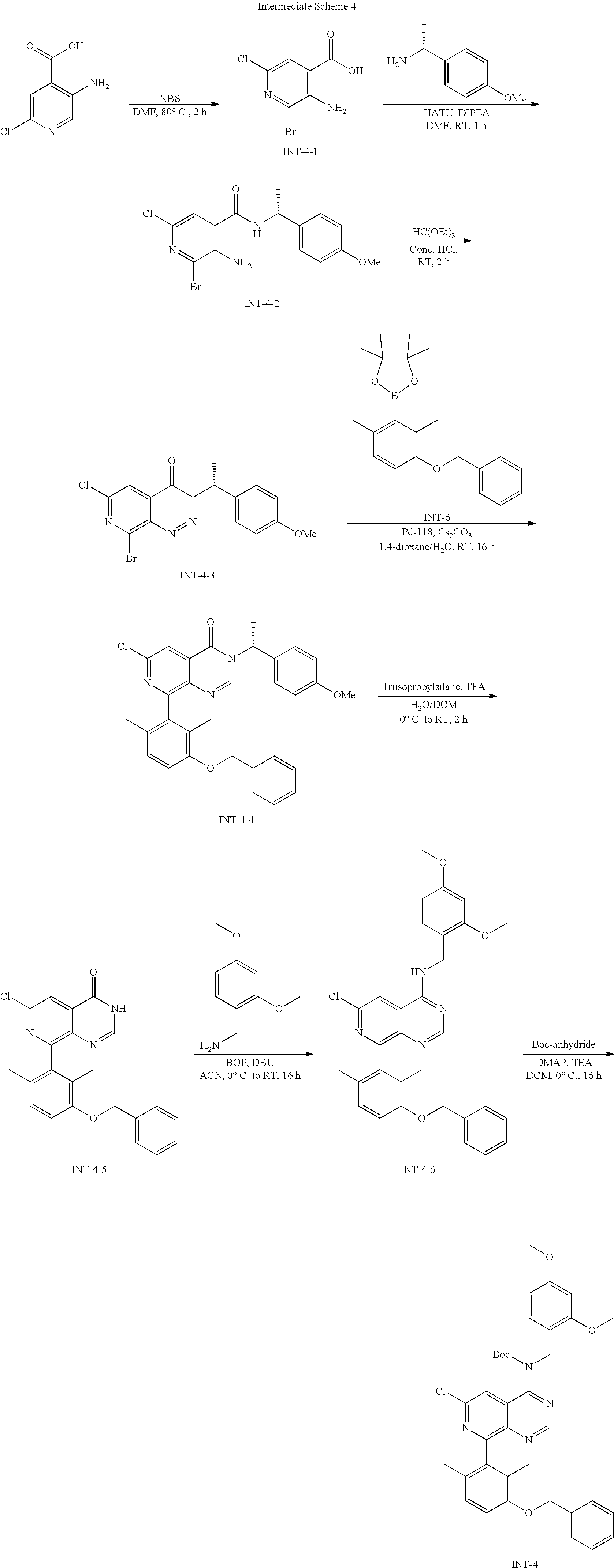

### 3-amino-2-bromo-6-chloroisonicotinic acid (INT-4-1)

To a solution of 5-amino-2-chloroisonicotinic acid (25 g, 144.87 mmol) in DMF (250 mL) was added N-bromosuccinimide (30.94 g, 173.85 mmol) at rt. After addition, the reaction mixture was stirred at 80° C. for 2 h. After completion, the reaction mixture was cooled to rt and diluted with ice cold water (500 mL). A product formed and the resulting product was filtered. The product was dried under vacuum for 1 h. The crude was triturated with pentane (2×250 mL) to get methyl 3-amino-2-bromo-6-chloroisonicotinate (INT-4-1) (29 g, 80%), which was used in the next step without further purification. MS (ESI) m/z: 251.03 [M+H]⁺.

### (R)-3-amino-2-bromo-6-chloro-N-(1-(4-methoxyphenyl)ethyl)isonicotinamide (INT-4-2)

To a solution of 3-amino-2-bromo-6-chloroisonicotinic acid (INT-4-1) (80 g, 318.14 mmol) in DMF (800 mL) were sequentially added HATU (120.97 g, 318.14 mmol), DIPEA (277.08 mL, 1590.69 mmol), and (R)-1-(4-methoxyphenyl)ethan-1-amine (52.92 g, 349.95 mmol) at 0° C. After addition, the reaction mixture was stirred at rt for 1 h. After completion, the reaction mixture was diluted with water (1000 mL). A product formed and the resulting product was filtered. The product was dried under vacuum to afford (R)-3-amino-2-bromo-6-chloro-N-(1-(4-methoxyphenyl)ethyl)isonicotinamide (INT-4-2) (85 g, 69%). MS (ESI) m/z: 382.3 [M−H]⁻.

### (R)-8-bromo-6-chloro-3-(1-(4-methoxyphenyl)ethyl)pyrido[3,4-d]pyrimidin-4 (3H)-one (INT-4-3)

To a solution of (R)-3-amino-2-bromo-6-chloro-N-(1-(4-methoxyphenyl)ethyl)isonicotinamide (INT-4-2) (14 g, 36.39 mmol) in triethyl orthoformate (280 mL) was added concentrated HCl (4.46 mL, 145.58 mmol) at 0° C. After addition, the reaction mixture was stirred at rt for 2 h. After completion, the low volatiles were concentrated. To the residue EtOAc (100 mL) was added, washed with sat. NaHCO₃ (3×150 mL) and water (2×150 mL). The organic layer was dried over Na₂SO₄, filtered, and concentrated to afford (R)-8-bromo-6-chloro-3-(1-(4-methoxyphenyl)ethyl)pyrido[3,4-d]pyrimidin-4 (3H)-one (INT-4-3) (20 g, 78%). MS (ESI) m/z: 392.1 [M−H]⁻.

### 8-(3-(benzyloxy)-2,6-dimethylphenyl)-6-chloro-3-((R)-1-(4-methoxyphenyl)ethyl) pyrido[3,4-d]pyrimidin-4 (3H)-one (INT-4-4)

To a solution of (R)-8-bromo-6-chloro-3-(1-(4-methoxyphenyl)ethyl)pyrido[3,4-d]pyrimidin-4 (3H)-one (INT-4-3) (4.0 g, 10.14 mmol) and 2-(3-(benzyloxy)-2,6-dimethylphenyl)-4,4,5,5-tetramethyl-1,3,2-dioxaborolane (INT-6) (3.94 g, 11.66 mmol) in 1,4 dioxane (64 mL) and water (16 mL) was added Cs₂CO₃ (9.91 g, 30.41 mmol) at rt. The mixture was purged with nitrogen for 10 min. To this mixture, Pd(dppf)Cl₂ (0.66 g, 1.01 mmol) was added and the mixture purged again with nitrogen for 10 min. The reaction mixture was stirred at 90° C. for 16 h. After completion, water (50 mL) was added to the reaction mixture and extracted with EtOAc (2×100 mL). The combined organic layer was washed with brine (100 mL), dried over Na₂SO₄, filtered, and concentrated. The residue was purified by preparative HPLC (ACN/H₂O with 0.1% formic acid) to afford 8-(3-(benzyloxy)-2,6-dimethylphenyl)-6-chloro-3-((R)-1-(4-methoxyphenyl)ethyl)pyrido[3,4-d]pyrimidin-4 (3H)-one (INT-4-4) (1.5 g, 28%). MS (ESI) m/z: 526.4 [M+H]⁺.

### 8-(3-(benzyloxy)-2,6-dimethylphenyl)-6-chloropyrido[3,4-d]pyrimidin-4 (3H)-one (INT-4-5)

To a solution of 8-(3-(benzyloxy)-2,6-dimethylphenyl)-6-chloro-3-((R)-1-(4-methoxyphenyl)ethyl)pyrido[3,4-d]pyrimidin-4 (3H)-one (INT-4-4) (4.0 g, 7.60 mmol) in DCM (80 mL) and water (4 mL) were added triisopropylsilane (1.45 g, 9.13 mmol) and TFA (40 mL) at 0° C. After addition, the reaction mixture was stirred at rt for 2 h. After completion, the reaction mixture was concentrated. The residue was diluted with DCM (50 mL), washed with water (50 mL), sat. NaHCO₃ (50 mL), and brine (50 mL). The combined organic layers were dried over Na₂SO₄, filtered, and concentrated. The residue was purified by column chromatography (SiO₂, EtOAc/pet ether) to afford 8-(3-(benzyloxy)-2,6-dimethylphenyl)-6-chloropyrido[3,4-d]pyrimidin-4 (3H)-one (INT-4-5) (1.9 g, 57%). MS (ESI) m/z: 392.4 [M+H]⁺.

### 8-(3-(benzyloxy)-2,6-dimethylphenyl)-6-chloro-N-(2,4-dimethoxybenzyl)pyrido[3,4-d]pyrimidin-4-amine (INT-4-6)

To a solution of 8-(3-(benzyloxy)-2,6-dimethylphenyl)-6-fluoropyrido[3,4-d]pyrimidin-4 (3H)-one (INT-4-5) (1.9 g, 4.85 mmol) in ACN (19 mL) were added (2,4-dimethoxyphenyl)methanamine (1.62 g, 9.70 mmol), BOP reagent (6.43 g, 14.55 mmol) and DBU (3.69 g, 24.24 mmol) at rt. After addition, the reaction mixture was stirred at rt for 16 h. After completion, the reaction mixture was carefully quenched with water (50 mL) and extracted with EtOAc (2×50 mL). The combined organic layers were washed with brine (50 mL), dried over Na₂SO₄, filtered, and concentrated. The residue was purified by column chromatography (SiO₂, EtOAc/pet ether) to afford 8-(3-(benzyloxy)-2,6-dimethylphenyl)-N-(2,4-dimethoxybenzyl)-6-fluoropyrido[3,4-d]pyrimidin-4-amine (INT-4-6) (1.9 g, 72%). MS (ESI) m/z: 541.46 [M+H]⁺.

### tertbutyl (8-(3-(benzyloxy)-2,6-dimethylphenyl)-6-chloropyrido[3,4-d]pyrimidin-4-yl)(2,4-dimethoxybenzyl)carbamate (INT-4)

To a solution of 8-(3-(benzyloxy)-2,6-dimethylphenyl)-6-chloro-N-(2,4-dimethoxybenzyl)pyrido[3,4-d]pyrimidin-4-amine (INT-4-6) (2.0 g, 3.69 mmol) in DCM (20 mL) were added DMAP (0.09 g, 0.74 mmol), Et₃N (0.75 g, 7.39 mmol), and di-tert-butyl dicarbonate (1.61 g, 7.39 mmol) at 0° C. After addition, the reaction mixture was stirred at rt for 16 h. After completion, the reaction was carefully quenched with water (50 mL) and extracted with EtOAc (2×50 mL). The combined organic layers were washed with brine (50 mL), dried over Na₂SO₄, filtered, and concentrated. The residue was purified by column chromatography (SiO₂, EtOAc/pet ether) to afford tert-butyl (8-(3-(benzyloxy)-2,6-dimethylphenyl)-6-chloropyrido[3,4-d]pyrimidin-4-yl)(2,4-dimethoxybenzyl)carbamate (INT-4) (2 g, 84%). ¹H NMR (400 MHz, CDCl₃): δ ppm 9.19 (s, 1H), 7.51 (s, 1H), 7.45 (d, J=7.2 Hz, 2H), 7.38 (t, J=7.2 Hz, 2H), 7.31 (t, J=8.4 Hz, 2H), 7.10 (d, J=8.4 Hz, 1H), 6.95 (d, J=8.4 Hz, 1H), 6.43 (dd, J=8.4, 2.0 Hz, 1H), 6.30 (d, J=2.0 Hz, 1H), 5.09 (s, 4H), 3.78 (s, 3H), 3.27 (s, 3H), 1.85 (s, 3H), 1.84 (s, 3H), 1.37 (s, 9H); MS (ESI) m/z: 641.6 [M+H]⁺.

### Intermediate 5

### (3-Methoxy-2,6-dimethylphenyl)boronic acid (INT-5)

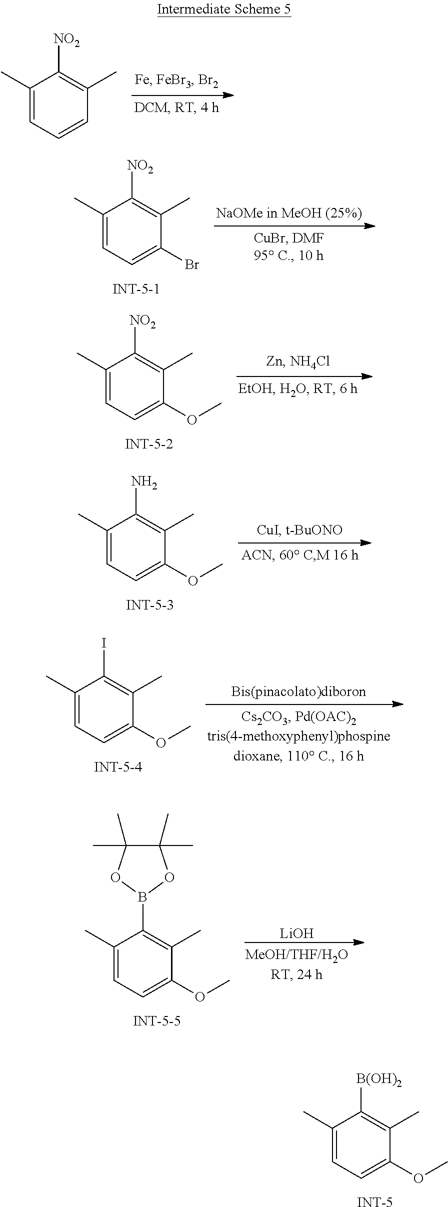

### 1-bromo-2,4-dimethyl-3-nitrobenzene (INT-5-1)

To a solution of 1,3-dimethyl-2-nitrobenzene (100 g, 661.53 mmol) in DCM (1000 mL) were added Fe (9.23 g, 165.38 mmol) and FeBr₃ (3.910 g, 13.23 mmol) at rt and stirred for 5 min. The mixture was cooled to −15° C. and Br₂ (36.34 mL, 727.68 mmol) was added dropwise over 45 min. After addition, the mixture was stirred at rt for 3 h. After completion, the mixture was diluted with cold water (500 mL) and extracted with Et₂O (2×500 mL). The combined organic layer was washed with saturated Na₂S₂O₃ (500 mL), brine (500 mL), dried over Na₂SO₄, filtered over a silica gel bed (230-400), and concentrated to obtain 1-bromo-2,4-dimethyl-3-nitrobenzene (INT-5-1) (140 g, 92%). The product was used for the next step without further purification. ¹H NMR (400 MHz, CDCl₃): δ ppm 7.55 (d, J=8.4 Hz, 1H), 7.01 (d, J=8.4 Hz, 1H), 2.34 (s, 3H), 2.25 (s, 3H).

### 1-methoxy-2,4-dimethyl-3-nitrobenzene (INT-5-2)

To a solution of 1-bromo-2,4-dimethyl-3-nitrobenzene (INT-5-1) (100 g, 434.67 mmol) in DMF (400 mL) was added CuBr (12.471 g, 86.933 mmol) followed by addition of 25% NaOMe in MeOH (300 mL, 1304 mmol). The mixture was stirred at rt for 10 min and then at 100° C. for 16 h. After completion, the mixture was diluted with water (300 mL) and extracted with EtOAc (3×300 mL). The combined organic layer was washed with brine (300 mL), dried over Na₂SO₄, filtered, and concentrated to obtain the 1-methoxy-2,4-dimethyl-3-nitrobenzene (INT-5-2) (70.0 g, 88%). The product was used for the next step without further purification. ¹H NMR (400 MHz, CDCl₃): δ ppm 7.08 (d, J=8.40 Hz, 1H), 6.86 (d, J=8.40 Hz, 1H), 3.87 (s, 3H), 2.25 (s, 3H), 2.16 (s, 3H).

### 3-methoxy-2,6-dimethylaniline (INT-5-3)

To a solution of 1-methoxy-2,4-dimethyl-3-nitrobenzene (INT-5-2) (100 g, 549.72 mmol) in a mixture of EtOH (600 mL) and water (200 mL) were added Zn (215.64 g, 3298.334 mmol) and NH₄Cl (176.428 g, 3298.334 mmol) at rt. The mixture was stirred at rt for 6 h. After completion, the mixture was diluted with water (1000 mL) and extracted with DCM (3×1000 mL). The combined organic layer was washed with brine (1000 mL), dried over MgSO₄, filtered, and concentrated. The residue was purified by column chromatography (SiO₂, EtOAc/PET) to afford 3-methoxy-2,6-dimethylaniline (INT-5-3) (72 g, 86%). ¹H NMR (400 MHz, DMSO-d₆): δ ppm 6.73 (d, J=8.40 Hz, 1H), 6.14 (d, J=8.00 Hz, 1H), 4.48 (s, 2H), 3.66 (s, 3H), 2.01 (s, 3H), 1.92 (s, 3H); MS (ESI) m/z: 152.01 [M+H]⁺.

### 2-iodo-4-methoxy-1,3-dimethylbenzene (INT-5-4)

To a solution of 3-methoxy-2,6-dimethylaniline (INT-5-3) (50.0 g, 330.68 mmol) in ACN (1000 mL) were added CuI (157.44 g, 826.69 mmol) and ^{t}BuONO (78.66 mL, 661.351 mmol) at rt. The mixture was stirred at 60° C. for 16 h. After completion, the mixture was diluted with water (1000 mL) and extracted with EtOAc (4×500 mL). The combined organic layer was washed with saturated Na₂S₂O₃ (4×1000 mL), dried over MgSO₄, filtered, and concentrated. The residue was purified by column chromatography (SiO₂, EtOAc/PET) to afford 2-iodo-4-methoxy-1,3-dimethylbenzene (INT-5-4) (38.0 g, 43%). ¹H NMR (400 MHz, DMSO-d₆): δ ppm 7.16 (d, J=8.4 Hz, 1H), 6.90 (d, J=8.4 Hz, 1H), 3.77 (s, 3H), 2.34 (s, 3H), 2.26 (s, 3H).

### 2-(3-methoxy-2,6-dimethylphenyl)-4,4,5,5-tetramethyl-1,3,2-dioxaborolane (INT-5-5)

To a solution of 2-iodo-4-methoxy-1,3-dimethylbenzene (INT-5-4) (5.0 g, 19.08 mmol) in 1,4-dioxane (150 mL) were added Cs₂CO₃ (9.324 g, 28.62 mmol), Pd(OAc)₂ (0.086 g, 0.382 mmol), bis(pinacolato)diboron (7.267 g, 28.62 mmol), and tris(4-methoxyphenyl)phosphine (0.134 g, 0.382 mmol) at rt. The mixture was degassed with N₂ for 5 min and stirred at 90° C. for 16 h. After completion, the mixture was diluted with DCM (100 mL) and filtered through a celite pad, washed with DCM (2×100 mL). The filtrate was concentrated. The residue was purified by column chromatography (SiO₂, EtOAc/PET) to afford 2-(3-methoxy-2,6-dimethylphenyl)-4,4,5,5-tetramethyl-1,3,2-dioxaborolane (INT-5-5) (4.1 g, 81%). ¹H NMR (400 MHz, DMSO-d₆): δ ppm 6.92 (d, J=8.0 Hz, 1H), 6.81 (d, J=8.4 Hz, 1H), 3.71 (s, 3H), 2.22 (s, 3H), 2.13 (s, 3H), 1.32 (s, 12H); MS (ESI) m/z: 263.32 [M+H]⁺.

### (3-methoxy-2,6-dimethylphenyl)boronic acid (INT-5)

To a solution of 2-(3-methoxy-2,6-dimethylphenyl)-4,4,5,5-tetramethyl-1,3,2-dioxaborolane (INT-5-5) (27.0 g, 102.99 mmol) in a mixture of MeOH (135 mL), water (54 mL) and THF (135 mL) was added LiOH (43.215 g, 1029.9 mmol) at rt. The mixture was stirred at rt for 24 h. After completion, the mixture was acidified with 2N HCl (500 mL) and extracted with EtOAc (2×500 mL). The combined organic layer was washed with brine (500 mL), dried over MgSO₄, filtered, and concentrated. The residue was purified by column chromatography (SiO₂, EtOAc/PET) to afford (3-methoxy-2,6-dimethylphenyl)boronic acid (INT-5) (14 g, 75%). ¹H NMR (400 MHz, DMSO-d₆): δ ppm 8.10 (s, 2H), 6.89 (d, J=8.0 Hz, 1H), 6.73 (d, J=8.4 Hz, 1H), 3.71 (s, 3H), 2.18 (s, 3H), 2.09 (s, 3H); MS (ESI) m/z: 179.04 [M−H]⁺.

### Intermediate 6

### 2-(3-(benzyloxy)-2,6-dimethylphenyl)-4,4,5,5-tetramethyl-1,3,2-dioxaborolane (INT-6)

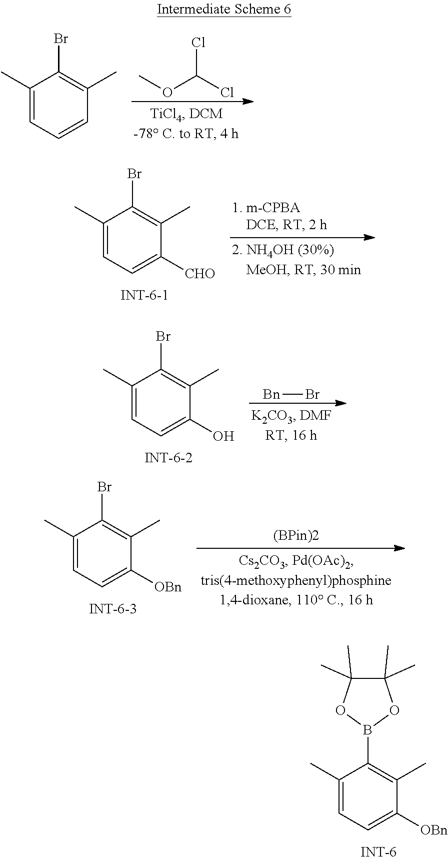

### 3-bromo-2,4-dimethylbenzaldehyde (INT-6-1)

To a solution of 2-bromo-1,3-dimethylbenzene (100 g, 540.35 mmol) in DCM (200 mL) was added TiCl₄ (120.60 mL, 1080.71 mmol) at −78° C. To this mixture, a solution of dichloro(methoxy)methane (53.76 mL, 594.39 mmol) in DCM (300 mL) was added dropwise. After addition, the reaction mixture was stirred at rt for 16 h. After completion, the reaction mixture was quenched with ice cold water (500 mL) and extracted with DCM (2×2 L). The combined organic layer was dried over Na₂SO₄, filtered, and concentrated to afford 3-bromo-2,4-dimethylbenzaldehyde (INT-6-1) (115 g, 99.88%). The crude material was used for next step without further purification. ¹H NMR (400 MHz, CDCl₃): δ ppm 10.22 (s, 1H), 7.67 (d, J=8.0 Hz, 1H), 7.07 (dd, J=7.2, 4.4 Hz, 1H), 2.50 (s, 3H), 2.41 (s, 3H).

### 3-bromo-2,4-dimethylphenol (INT-6-2)

To a solution of 3-bromo-2,4-dimethylbenzaldehyde (INT-6-1) (115 g, 539.72 mmol) in DCE (400 mL) was added m-CPBA (139.709 g, 809.578 mmol) at 0° C. The reaction mixture was stirred at 45° C. for 16 h. After completion, the reaction mixture was diluted with water (500 mL) and quenched with saturated NaHCO₃ (500 mL). The crude was extracted with DCM (2×2 L). The combined organic layer was washed with brine (2 L), dried over Na₂SO₄, filtered, and concentrated. The crude was diluted with MeOH (300 mL) and aq. NH₄OH (25%, 100 mL) was added at 0° C. The reaction mixture was stirred at 0° C. for 30 min and quenched with ice cold water (1 L). The crudes were extracted with DCM (2×2 L). The combined organic layer was concentrated to afford 3-bromo-2,4-dimethylphenol (INT-6-2) (85 g, 78%). The crude material was used for the next step without further purification. ¹H NMR (400 MHz, CDCl₃): δ ppm 9.55 (s, 1H), 6.97 (d, J=8.4 Hz, 1H), 6.74 (d, J 8.0 Hz, 1H), 2.25 (s, 3H), 2.22 (s, 3H).

### 1-(benzyloxy)-3-bromo-2,4-dimethylbenzene (INT-6-3)

To a solution of 3-bromo-2,4-dimethylphenol (INT-6-2) (80 g, 397.812 mmol) in DMF (160 mL) was added K₂CO₃ (164.933 g, 1193.436 mmol) at rt. The mixture was cooled to 0° C. and benzyl bromide (94.5 mL, 795.62 mmol) was added. After addition, the reaction mixture was stirred at rt for 16 h. After completion, the reaction mixture was quenched with ice cold water (500 mL) and extracted with EtOAc (3×500 mL). The combined organic layer was dried over Na₂SO₄, filtered, and concentrated. The residue was purified by column chromatography (SiO₂, pet ether) to afford 1-(benzyloxy)-3-bromo-2,4-dimethylbenzene (INT-6-3) (72 g, 62%). ¹H NMR (400 MHz, CDCl₃): δ ppm 7.51-7.35 (m, 5H), 7.01 (d, J=8.4 Hz, 1H), 6.76 (d, J=8.0 Hz, 1H), 5.04 (s, 2H), 2.40 (s, 3H), 2.36 (d, J=5.6 Hz, 3H).

### 2-(3-(benzyloxy)-2,6-dimethylphenyl)-4,4,5,5-tetramethyl-1,3,2-dioxaborolane (INT-6)

To a solution of 1-(benzyloxy)-3-bromo-2,4-dimethylbenzene (INT-6-3) (20 g, 68.684 mmol) in 1,4-dioxane (225 mL) were added bis(pinacolato)diboron (26.162 g, 103.026 mmol) and Cs₂CO₃ (33.568 g, 103.026 mmol) at rt. The mixture was degassed with nitrogen for 30 min. To this mixture, Pd(OAc)₂ (2.313 g, 3.434 mmol) and tris(4-methoxyphenyl)phosphine (1.210 g, 3.434 mmol) were added. The reaction mixture was degassed again with nitrogen for 10 minutes and stirred at 110° C. for 16 h. After completion, the reaction mixture was quenched with water (300 mL) and extracted with EtOAc (2×400 mL). The combined organic layer was washed with brine (200 mL), dried over MgSO₄, filtered, and concentrated. The residue was purified by column chromatography (SiO₂, EtOAc/pet ether) to afford 2-(3-(benzyloxy)-2,6-dimethylphenyl)-4,4,5,5-tetramethyl-1,3,2-dioxaborolane (INT-6) (34.0 g, 83%). ¹H NMR (400 MHz, CDCl₃): δ ppm 7.44-7.36 (m, 4H), 7.31 (d, J=7.2 Hz, 1H), 6.89 (s, 1H), 5.07 (s, 2H), 2.21 (d, J=9.2 Hz, 6H), 1.33 (s, 12H); MS (ESI) 339.43 [M+H]⁺.

### Intermediate 7

### 6-fluoro-8-(3-methoxy-2,6-dimethylphenyl)-3-((R)-1-(4-methoxyphenyl)ethyl)pyrido[3,4-d]pyrimidin-3 (3H)-one (INT-7)

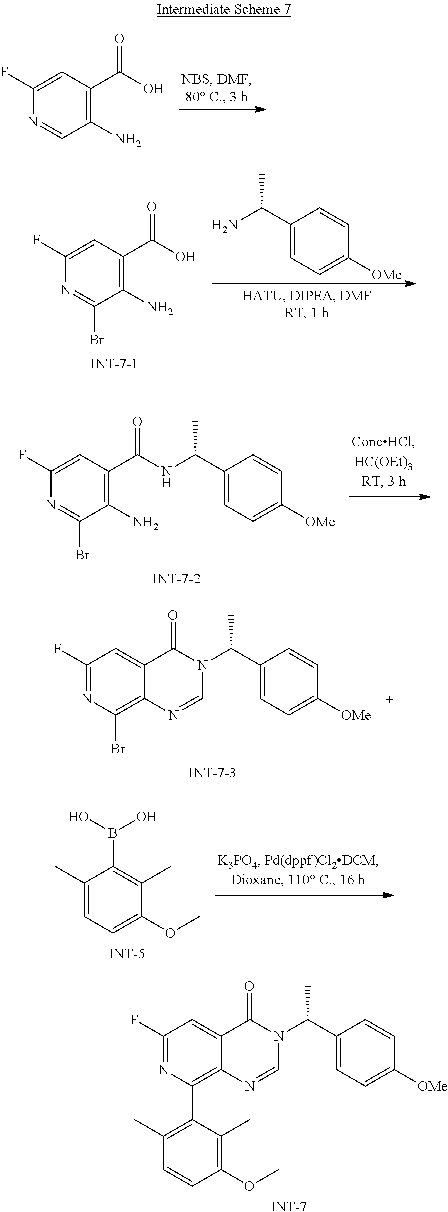

### 3-amino-2-bromo-6-fluoroisonicotinic acid (INT-7-1)

To a stirred solution of 5-amino-2-chloroisonicotinic acid (38.0 g, 243 mmol) in DMF (380 mL) was added NBS (52.0 g, 292 mmol) at rt and the mixture was heated at 80° C. for 3 h. The mixture was poured into ice-cold water (1000 mL). The precipitated product was collected by filtration and dried under vacuum to afford 3-amino-2-bromo-6-fluoroisonicotinic acid (INT-7-1) (26.0 g, 74%), which was used directly for the next step without further purification. ¹H NMR (400 MHz, CDCl₃) δ ppm 13.59 (s, 1H), 8.50-5.80 (brs, 2H), 7.39 (d, J=2.4 Hz, 1H); MS (ESI) 235.1 [M+H]⁺.

### (R)-3-amino-2-bromo-6-fluoro-N-(1-(4-methoxyphenyl)ethyl)isonicotinamide (INT-7-2)

To a stirred solution of 3-amino-2-bromo-6-fluoroisonicotinic acid (INT-7-1) (26.0 g, 111 mmol) in DMF (260 mL) were added HATU (42.1 g, 111 mmol), N,N-diisopropylethylamine (96.4 mL, 553 mmol) and (R)-1-(4-methoxyphenyl)ethan-1-amine (18.4 g, 122 mmol) sequentially at 0° C. The mixture warmed up to rt and stirred at rt for 1 h. The mixture was diluted with ice cold water (750 mL). The precipitated product was collected by filtration and dried under vacuum to afford (R)-3-amino-2-bromo-6-fluoro-N-(1-(4-methoxyphenyl)ethyl)isonicotinamide (INT-7-2) (36.0 g, 88%), which was used directly for the next step without further purification. ¹H NMR (400 MHz, CDCl₃) δ ppm 9.06 (d, J=7.6 Hz, 1H), 7.42 (s, 1H), 7.30 (d, J=8.4 Hz, 2H), 6.89 (d, J=8.8 Hz, 2H), 6.17 (s, 2H), 5.09-5.05 (m, 1H), 3.73 (s, 3H), 1.44 (d, J=6.8 Hz, 3H); MS (ESI) 368.17 [M+H]⁺.

### (R)-8-bromo-6-fluoro-3-(1-(4-methoxyphenyl)ethyl)pyrido[3,4-d]pyrimidin-4 (3H)-one (INT-7-3)

To a stirred solution of (R)-3-amino-2-bromo-6-fluoro-N-(1-(4-methoxyphenyl)ethyl)isonicotinamide (INT-7-2) (36.0 g, 97.8 mmol) in triethyl orthoformate (360 mL) was added hydrochloric acid (16.2 mL, 196 mmol) at 0° C. The mixture was warmed to rt and stirred at rt for 3 h. The mixture was concentrated, diluted with EtOAc (750 mL), washed with water (2×300 mL) and saturated aq. NaHCO₃ solution (2×400 mL), dried over anhydrous MgSO₄, filtered and concentrated. The residue was purified by flash column chromatography (SiO₂, EtOAc/pet ether gradient) to afford (R)-8-bromo-6-fluoro-3-(1-(4-methoxyphenyl)ethyl)pyrido[3,4-d]pyrimidin-4 (3H)-one (INT-7-3) (30.0 g, 81%). ¹H NMR (400 MHz, CDCl₃) δ ppm 8.59 (s, 1H), 7.76 (d, J=2.8 Hz, 1H), 7.39 (d, J=8.8 Hz, 2H), 6.93 (d, J=8.8 Hz, 2H), 5.99 (q, J=7.2 Hz, 1H), 3.74 (s, 3H), 1.83 (d, J=7.2 Hz, 3H). MS (ESI) m/z 378.5 [M+H]⁺.

### 6-fluoro-8-(3-methoxy-2,6-dimethylphenyl)-3-((R)-1-(4-methoxyphenyl)ethyl)pyrido[3,4-d]pyrimidin-4 (3H)-one (INT-7)

To a stirred solution of (R)-8-bromo-6-fluoro-3-(1-(4-methoxyphenyl)ethyl)pyrido[3,4-d]pyrimidin-4 (3H)-one (INT-7-3) (1.00 g, 2.64 mmol) and (3-methoxy-2,6-dimethylphenyl)boronic acid (INT-5) (0.476 g, 2.64 mmol) in dioxane (20.0 mL) and water (3.0 mL), was added potassium phosphate (1.68 g, 7.93 mmol), purged with nitrogen for 5 minutes followed by addition of Pd(dppf)Cl₂·DCM (0.24-62 g, 0.264 mmol). After the mixture purged again for 3 minutes and stirred at 110° C. for 16 h, it was quenched with water (100 mL), extracted with EA (2×100 mL), combined organic layers washed with brine (50 mL), dried over sodium sulphate, filtered, concentrated and the residue purified by flash column chromatography (SiO₂, EA/PET gradient) to afford 6-fluoro-8-(3-methoxy-2,6-dimethylphenyl)-3-((R)-1-(4-methoxyphenyl)ethyl)pyrido[3,4-d]pyrimidin-4 (3H)-one (INT-7) (0.84 g, 73%). MS (ESI) 434.20 [M+H]⁺.

### Intermediate 8

### tert-butyl (8-(3-(benzyloxy)-2,6-dimethylphenyl)-6-fluoropyrido[3,4-d]pyrimidin-4-yl)(2,4-dimethoxybenzyl)carbamate (INT-8)

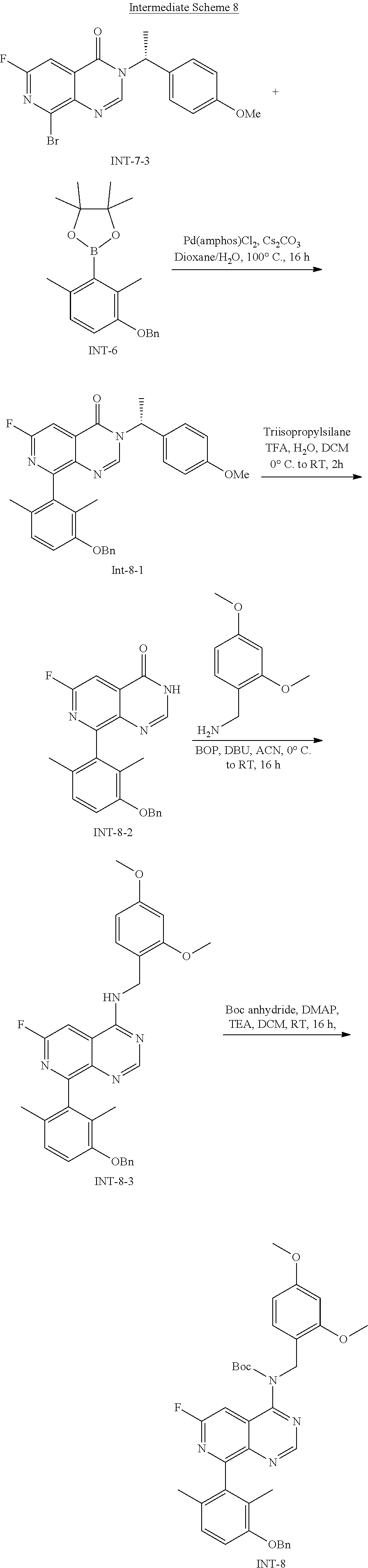

### 8-(3-(benzyloxy)-2,6-dimethylphenyl)-6-fluoro-3-((R)-1-(4-methoxyphenyl)ethyl)pyrido[3,4-d]pyrimidin-4 (3H)-one (INT-8-1)

To a stirred solution of (R)-8-bromo-6-fluoro-3-(1-(4-methoxyphenyl)ethyl)pyrido[3,4-d]pyrimidin-4 (3H)-one (INT-7-3) (18.0 g, 47.6 mmol) and 2-(3-(benzyloxy)-2,6-dimethylphenyl)-4,4,5,5-tetramethyl-1,3,2-dioxaborolane (INT-6) (24.1 g, 71.3 mmol) in 1,4-dioxane/water (4:1, 180 mL) was added cesium carbonate (31.0 g, 95.2 mmol). The mixture was purged with nitrogen for 5 min, followed by addition of bis(di-tert-butyl(4-dimethylaminophenyl) phosphine)dichloropalladium(II) (5.05 g, 7.14 mmol). After degassing again for 3 min and heating at 100° C. for 16 h, the mixture was diluted with water (300 mL) and extracted with EtOAc (2×300 mL). The combined organic layers washed with brine (300 mL), dried over anhydrous sodium sulphate and concentrated. The residue purified by flash column chromatography (SiO₂, EA/PET gradient) to afford 8-(3-(benzyloxy)-2,6-dimethylphenyl)-6-fluoro-3-((R)-1-(4-methoxyphenyl)ethyl)pyrido[3,4-d]pyrimidin-4 (3H)-one (INT-8-1) (13.8 g, 56%). ¹H NMR (400 MHz, CDCl₃) δ ppm 7.89 (s, 1H), 7.76-7.75 (m, 1H), 7.44-7.41 (m, 2H), 7.39-7.31 (m, 2H), 7.29-7.26 (m, 3H), 7.04 (t, J=8.00 Hz, 1H), 6.91-6.87 (m, 3H), 6.23 (q, J=7.2 Hz, 1H), 5.07 (d, J=6.4 Hz, 2H), 3.79 (s, 3H), 1.90 (s, 3H), 1.85 (s, 3H), 1.81 (d, J=7.20 Hz, 3H); MS (ESI) 510.31 [M+H]⁺.

### 8-(3-(benzyloxy)-2,6-dimethylphenyl)-6-fluoropyrido[3,4-d]pyrimidin-4 (3H)-one (INT-8-2)

To a stirred solution of 8-(3-(benzyloxy)-2,6-dimethylphenyl)-6-fluoro-3-((R)-1-(4-methoxyphenyl)ethyl)pyrido[3,4-d]pyrimidin-4 (3H)-one (INT-8-1) (13.8 g, 27.1 mmol) in DCM (270 mL) were added trifluoroacetic acid (128 mL), water (13.8 mL) and triisopropylsilane (6.66 mL, 32.5 mmol) at RT and the mixture stirred at RT for 2 h. The reaction was quenched with saturated sodium bicarbonate solution (200 mL) and extracted with EA (3×150 mL). The combined organic layers washed with brine (200 mL), dried over magnesium sulfate, filtered and concentrated. The residue purified by flash column chromatography (SiO₂, EA/PET gradient) to afford 8-(3-(benzyloxy)-2,6-dimethylphenyl)-6-fluoropyrido[3,4-d]pyrimidin-4 (3H)-one (INT-8-2) (6.45 g, 63%). ¹H NMR (400 MHz, CDCl₃) δ ppm 10.26 (bs, 1H), 7.97 (s, 1H), 7.74-7.73 (m, 1H), 7.45-7.433 (m, 2H), 7.40-7.36 (m, 2H), 7.33-7.31 (m, 1H), 7.09 (d, J=8.40 Hz, 1H), 6.93 (d, J=8.40 Hz, 1H), 5.09 (s, 2H), 1.91 (s, 3H), 1.90 (s, 3H); MS (ESI) 376.19 [M+H]⁺.

### 8-(3-(benzyloxy)-2,6-dimethylphenyl)-N-(2,4-dimethoxybenzyl)-6-fluoropyrido[3,4-d]pyrimidin-4-amine (INT-8-3)

To a stirred solution of 8-(3-(benzyloxy)-2,6-dimethylphenyl)-6-fluoropyrido[3,4-d]pyrimidin-4 (3H)-one (INT-8-2) (6.45 g, 14.2 mmol) and (2,4-dimethoxyphenyl)methanamine (4.74 g, 28.4 mmol) in acetonitrile (130 mL) was added BOP reagent (18.8 g, 42.6 mmol) followed by DBU (10.6 mL, 71.0 mmol) at rt. After stirring at rt for 16 h, the mixture was diluted with water (100 mL) and extracted with EtOAc (2×300 mL). The combined organic layers washed with brine, dried over MgSO₄, filtered and concentrated. The residue purified by flash column chromatography (SiO₂, EA/PET gradient) to afford 8-(3-(benzyloxy)-2,6-dimethylphenyl)-N-(2,4-dimethoxybenzyl)-6-fluoropyrido[3,4-d]pyrimidin-4-amine (INT-8-3) (5.5 g, 61%). ¹H NMR (400 MHz, CDCl₃) δ ppm 8.69 (s, 1H), 7.46-7.44 (m, 2H), 7.39-7.28 (m, 4H), 7.08-7.05 (m, 2H), 6.92-6.89 (m, 1H), 6.55-6.48 (m, 2H), 6.26-6.24 (m, 1H), 5.09 (s, 2H), 4.81 (d, J=5.2 Hz, 2H), 3.93 (s, 3H), 3.82 (s, 3H), 1.87 (s, 6H); MS (ESI) 525.34 [M+H]⁺.

### tert-butyl (8-(3-(benzyloxy)-2,6-dimethylphenyl)-6-fluoropyrido[3,4-d]pyrimidin-4-yl)(2,4-dimethoxybenzyl)carbamate (INT-8)

To a stirred solution of 8-(3-(benzyloxy)-2,6-dimethylphenyl)-N-(2,4-dimethoxybenzyl)-6-fluoropyrido[3,4-d]pyrimidin-4-amine (INT-8-3) (5.5 g, 10.5 mmol) in DCM (55 mL) were added 4-Dimethylaminopyridine (0.26 g, 2.10 mmol) and triethylamine (2.92 mL, 21.0 mmol) followed by di-tert-butyl dicarbonate (4.81 mL, 21.0 mmol) at 0° C. After stirring at rt for 16 h, the mixture was diluted with water (100 mL) and extracted with EtOAc (2×100 mL). The combined organic layers washed with brine, dried over MgSO₄, filtered and concentrated. The residue purified by flash column chromatography (SiO₂, EA/PET gradient) to afford tert-butyl (8-(3-(benzyloxy)-2,6-dimethylphenyl)-6-fluoropyrido[3,4-d]pyrimidin-4-yl)(2,4-dimethoxybenzyl)carbamate (INT-8) (5.1 g, 77%). ¹H NMR (400 MHz, CDCl₃) δ ppm 9.18 (s, 1H), 7.46-7.44 (m, 2H), 7.40-7.30 (m, 4H), 7.12-7.10 (m, 1H), 7.05-7.04 (m, 1H), 6.97-6.95 (m, 1H), 6.44 (dd, J=2.40 Hz, J=8.40 Hz, 1H), 6.28 (d, J=2.0 Hz, 1H), 5.11 (s, 2H), 5.10 (s, 2H), 3.78 (s, 3H), 3.21 (s, 3H), 1.85 (s, 3H), 1.84 (s, 3H), 1.38 (s, 9H); MS (ESI) 625.39 [M+H]⁺.

### Intermediate 9 and Intermediate 10

### 8-(3-(Benzyloxy)-2,6-dimethylphenyl)-6-chloro-N-(2,4-dimethoxybenzyl)pyrido[3,2-d]pyrimidin-4-amine (INT-9) and tert-butyl (8-(3-(benzyloxy)-2,6-dimethylphenyl)-6-chloropyrido[3,2-d]pyrimidin-4-yl)(2,4-dimethoxybenzyl)carbamate (INT-10)

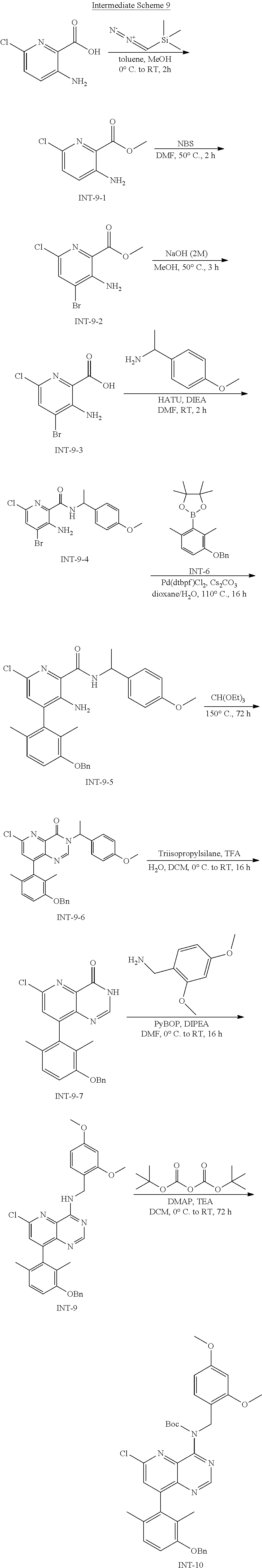

### Methyl 3-amino-6-chloropicolinate (INT-9-1)

At 0° C., to a stirred solution of 3-amino-6-chloropicolinic acid (10.0 g, 57.9 mmol) in a mixture of MeOH (30 mL) and toluene (90 mL) was added (diazomethyl)trimethylsilane (2.0 M in hexanes, 57.95 mL, 115.89 mmol). The resulting reaction mixture was stirred at rt for 2 h before it was treated with ice cold water (250 mL) and extracted with EtOAc (3×100 mL). The combined organic layers were dried over Na₂SO₄, filtered, and concentrated to afford methyl 3-amino-6-chloropicolinate (INT-9-1) (10.9 g, 81%), which was used for the next step without further purification. MS (ESI) m/z: 187.10 [M+H]⁺.

### Methyl 3-amino-4-bromo-6-chloropicolinate (INT-9-2)

At rt, to a stirred solution of methyl 3-amino-6-chloropicolinate (INT-9-1) (10.0 g, 53.6 mmol) in DMF (120 mL) was added NBS (14.3 g, 80.4 mmol). The resulting reaction mixture was stirred at 50° C. for 2 h, cooled to rt and quenched with ice cold water (700 mL). The product that was formed was collected and washed with ice cold water (200 mL) to afford methyl 3-amino-4-bromo-6-chloropicolinate (INT-9-2) (13.2 g, 86%), which was used for the next step without further purification. MS (ESI) m/z: 267.00 [M+H]⁺.

### 3-Amino-4-bromo-6-chloropicolinic acid (INT-9-3)

At 0° C., to a stirred solution of methyl 3-amino-4-bromo-6-chloropicolinate (INT-9-2) (13.0 g, 48.9 mmol) in MeOH (200 mL) was added aq. 2 N NaOH (100 mL). The resulting reaction mixture was stirred at 50° C. for 3 h, before being cooled to rt and concentrated. The residue was diluted with water (600 mL) and acidified with aq. 2 N HCl to adjust pH to 3.0. The product that formed were collected and washed with ice cold water (200 mL) to afford 3-amino-4-bromo-6-chloropicolinic acid (INT-9-3) (9.4 g, 85%), which was used for the next step without further purification. MS (ESI) m/z: 252.95 [M+H]⁺.

### 3-Amino-4-bromo-6-chloro-N-(1-(4-methoxyphenyl)ethyl)picolinamide (INT-9-4)

At 0° C., to a stirred solution of 3-amino-4-bromo-6-chloropicolinic acid (7.2 g, 28.6 mmol) in DMF (100 mL) were added HATU (10.9 g, 28.6 mmol), DIPEA (24.9 mL, 143.2 mmol) and 1-(4-methoxyphenyl)ethan-1-amine (INT-9-3) (4.8 g, 31.5 mmol). The resulting reaction mixture was stirred at rt for 2 h before being diluted with ice cold water (400 mL) and extracted with EtOAc (3×100 mL). The combined organic layers were washed with brine (3×50 mL), dried over Na₂SO₄, filtered and concentrated. The residue was purified by column chromatography (SiO₂, EtOAc/pet ether) to afford 3-amino-4-bromo-6-chloro-N-(1-(4-methoxyphenyl)ethyl)picolinamide (INT-9-4) (10.2 g, 92%). MS (ESI) m/z: 383.95 [M+H]⁻.

### 3-Amino-4-(3-(benzyloxy)-2,6-dimethylphenyl)-6-chloro-N-(1-(4-methoxyphenyl)ethyl)picolinamide (INT-9-5)

At rt, to a stirred solution of 3-amino-4-bromo-6-chloro-N-(1-(4-methoxyphenyl)ethyl) picolinamide (INT-9-4) (8.2 g, 21.3 mmol) in a mixture of 1,4-dioxane (200 mL) and water (50 mL) were added 2-(3-(benzyloxy)-2,6-dimethylphenyl)-4,4,5,5-tetramethyl-1,3,2-dioxaborolane (8.6 g, 25.6 mmol) and Cs₂CO₃ (13.9 g, 42.6 mmol). The mixture was purged with N₂ for 10 min followed by addition of [1,1′-bis(di-tert-butylphosphino)ferrocene]dichloropalladium(II) (0.97 g, 1.5 mmol) and stirred at 110° C. for 16 h. After completion, the reaction mixture was cooled to rt, diluted with EtOAc (300 mL) and washed with water (2×100 mL). The organic layers were dried over Na₂SO₄, filtered, and concentrated. The residue was purified by column chromatography (SiO₂, EtOAc/pet ether) to afford 3-amino-4-(3-(benzyloxy)-2,6-dimethylphenyl)-6-chloro-N-(1-(4-methoxyphenyl)ethyl)picolinamide (INT-9-5) (6.2 g, 56%). MS (ESI) m/z: 514.10 [M+H]⁻.

### 8-(3-(Benzyloxy)-2,6-dimethylphenyl)-6-chloro-3-(1-(4-methoxyphenyl)ethyl)pyrido[3,2-d]pyrimidin-4 (3H)-one (INT-9-6)

A solution of 3-amino-4-(3-(benzyloxy)-2,6-dimethylphenyl)-6-chloro-N-(1-(4-methoxyphenyl)ethyl)picolinamide (INT-9-5) (6.2 g, 12.0 mmol) in triethylorthoformate (120 mL) was stirred at 150° C. for 72 h. After completion, the reaction mixture was cooled to rt and diluted with EtOAc (150 mL). The organic layers were washed with water (2×50 mL) and brine (2×50 mL), dried over Na₂SO₄, filtered, and concentrated. The residue was purified by column chromatography (SiO₂, EtOAc/pet ether) to afford 8-(3-(benzyloxy)-2,6-dimethylphenyl)-6-chloro-3-(1-(4-methoxyphenyl)ethyl)pyrido[3,2-d]pyrimidin-4 (3H)-one (INT-9-6) (4.3 g, 68%). MS (ESI) m/z: 526.20 [M+H]⁺.

### 8-(3-(Benzyloxy)-2,6-dimethylphenyl)-6-chloropyrido[3,2-d]pyrimidin-4 (3H)-one (INT-9-7)

At 0° C., to a stirred solution of 8-(3-(benzyloxy)-2,6-dimethylphenyl)-6-chloro-3-(1-(4-methoxyphenyl)ethyl)pyrido[3,2-d]pyrimidin-4 (3H)-one (INT-9-6) (4.3 g, 8.2 mmol) in a mixture of DCM and (80 mL) and water (17.2 mL) were added TFA (80 mL) and triisopropylsilane (5.02 mL, 24.52 mmol). The resulting reaction mixture was stirred at rt for 16 h, quenched with saturated NaHCO₃ solution (100 mL) and extracted with EtOAc (3×100 mL). The combined organic layers were dried over Na₂SO₄, filtered and concentrated. The residue was purified by column chromatography (SiO₂, MeOH/DCM) to afford 8-(3-(benzyloxy)-2,6-dimethylphenyl)-6-chloropyrido[3,2-d]pyrimidin-4 (3H)-one (INT-9-7) (2.6 g, 81%). MS (ESI) m/z: 392.20 [M+H]⁺.

### 8-(3-(Benzyloxy)-2,6-dimethylphenyl)-6-chloro-N-(2,4-dimethoxybenzyl)pyrido[3,2-d]pyrimidin-4-amine (INT-9)

At 0° C., to a stirred solution of 8-(3-(benzyloxy)-2,6-dimethylphenyl)-6-chloropyrido[3,2-d]pyrimidin-4 (3H)-one (INT-9-7) (1.0 g, 2.6 mmol) and (2,4-dimethoxyphenyl)methanamine (0.8 g, 5.1 mmol) in DMF (40 mL) were added PyBOP (2.0 g, 3.8 mmol) and DIPEA (1.3 mL, 7.7 mmol). The resulting reaction mixture was stirred at rt for 16 h, treated with ice cold water (50 mL) and extracted with EtOAc (3×50 mL). The combined organic layers were washed with brine (3×20 mL), dried over Na₂SO₄, filtered, and concentrated. The residue was purified by column chromatography (SiO₂, EtOAc/pet ether) to afford 8-(3-(benzyloxy)-2,6-dimethylphenyl)-6-chloro-N-(2,4-dimethoxybenzyl)pyrido[3,2-d]pyrimidin-4-amine (INT-9) (1.3 g, 94%). MS (ESI) m/z: 541.35 [M+H]⁺.

### tert-butyl (8-(3-(benzyloxy)-2,6-dimethylphenyl)-6-chloropyrido[3,2-d]pyrimidin-4-yl)(2,4-dimethoxybenzyl)carbamate (INT-10)

At 0° C., to a stirred solution of 8-(3-(benzyloxy)-2,6-dimethylphenyl)-6-chloro-N-(2,4-dimethoxybenzyl)pyrido[3,2-d]pyrimidin-4-amine (INT-9) (1.0 g, 1.85 mmol) in DCM (20 mL) were added DMAP (0.045 g, 0.37 mmol), TEA (0.52 mL, 3.7 mmol) and di-tert-butyl dicarbonate (0.85 mL, 3.7 mmol). After addition, the reaction mixture was stirred at RT for 72 h. After completion, the reaction mixture was diluted with DCM (50 mL), washed with saturated Na₂CO₃ (2×20 mL) and water (2×20 mL). The organic layers were dried over Na₂SO₄, filtered, and concentrated. The residue was purified by column chromatography (SiO₂, EtOAc/pet ether) to afford tert-butyl (8-(3-(benzyloxy)-2,6-dimethylphenyl)-6-chloropyrido[3,2-d]pyrimidin-4-yl)(2,4-dimethoxybenzyl)carbamate (INT-10) (0.805 g, 67%). ¹H NMR (400 MHz, DMSO-d₆): δ ppm 9.04 (s, 1H), 7.97 (s, 1H), 7.48 (d, J=7.2 Hz, 2H), 7.40 (t, J=7.2 Hz, 2H), 7.36-7.21 (m, 2H), 7.14 (d, J=8.4 Hz, 1H), 7.09 (d, J=8.4 Hz, 1H), 6.49-6.45 (m, 2H), 5.15 (s, 2H), 5.05 (s, 2H), 3.72 (s, 3H), 3.41 (s, 3H), 1.78 (s, 3H), 1.75 (s, 3H), 1.25 (s, 9H); MS (ESI) m/z: 641.22 [M+H]⁺.

### Example 1

### (S)-3-(4-Amino-6-(methylamino)pyrido[3,4-d]pyrimidin-8-yl)-2,4-dimethylphenol (1-B) and (R)-3-(4-amino-6-(methylamino)pyrido[3,4-d]pyrimidin-8-yl)-2,4-dimethylphenol (1-C)

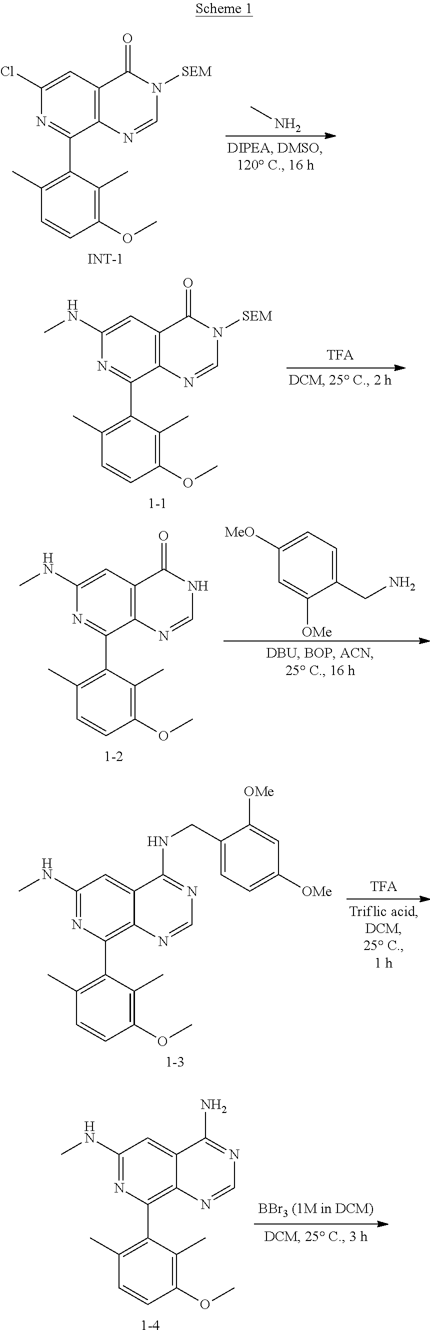

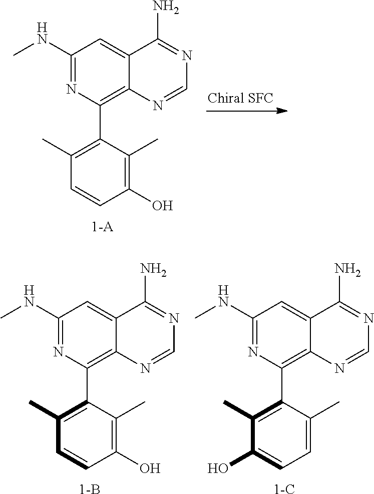

### 8-(3-methoxy-2,6-dimethylphenyl)-6-(methylamino)-3-((2-(trimethylsilyl)ethoxy) methyl)pyrido[3,4-d]pyrimidin-4 (3H)-one (1-1)

To a solution of 6-chloro-8-(3-methoxy-2,6-dimethylphenyl)-3-((2-(trimethylsilyl)ethoxy)methyl)pyrido[3,4-d]pyrimidin-4 (3H)-one (INT-1) (0.50 g, 1.12 mmol) in DMSO (5 mL) were added methanamine hydrochloride (2.27 g, 33.63 mmol), DIPEA (12.42 mL, 67.26 mmol) at rt. The mixture was stirred at 120° C. for 16 h. After completion, the reaction was quenched with water (50 mL) and extracted with EtOAc (3×50 mL). The combined organic layers were washed with brine (50 mL), dried over Na₂SO₄, filtered, and concentrated. The residue was purified by column chromatography (SiO₂, EtOAc/PET) to afford 8-(3-methoxy-2,6-dimethylphenyl)-6-(methylamino)-3-((2-(trimethylsilyl)ethoxy)methyl)pyrido[3,4-d]pyrimidin-4 (3H)-one (1-1) (0.20 g, 40%). MS (ESI) 441.3 [M+H]⁺.

### 8-(3-methoxy-2,6-dimethylphenyl)-6-(methylamino)pyrido[3,4-d]pyrimidin-4 (3H)-one (1-2)

To a solution of 8-(3-methoxy-2,6-dimethylphenyl)-6-(methylamino)-3-((2-(trimethylsilyl)ethoxy)methyl)pyrido[3,4-d]pyrimidin-4 (3H)-one (1-1) (0.52 g, 1.18 mmol) in CH₂Cl₂ (10.4 mL) was added TFA (5.20 mL, 0.09 mmol) at rt then stirred for 16 h. After completion, the reaction was quenched with sat. NaHCO₃ (25 mL) and extracted with EtOAc (2×25 mL). The combined organic layers were washed with brine (25 mL), dried over Na₂SO₄, filtered, and concentrated. The residue was purified by column chromatography (SiO₂, EtOAc/PET) to afford 8-(3-methoxy-2,6-dimethylphenyl)-6-(methylamino)pyrido[3,4-d]pyrimidin-4 (3H)-one (1-2) (0.72 g, quantitative). MS (ESI) m/z: 311.4 [M+H]⁺.

### N⁴-(2,4-dimethoxybenzyl)-8-(3-methoxy-2,6-dimethylphenyl)-N⁶-methylpyrido[3,4-d]pyrimidine-4,6-diamine (1-3)

To a solution of 8-(3-methoxy-2,6-dimethylphenyl)-6-(methylamino)pyrido[3,4-d]pyrimidin-4 (3H)-one (1-2) (0.72 g, 2.32 mmol) and (2,4-dimethoxyphenyl)methanamine (0.77 g, 4.64 mmol) in ACN (14.4 mL)) was degassed with N₂ for 15 min. followed by addition of DBU (0.71 g, 4.64 mmol) and BOP (1.54 g, 3.48 mmol). The mixture was stirred at rt for 16 h. After completion, water (25 mL) was added then extracted with EtOAc (3×25 mL). The combined organic layers were washed with brine (25 mL), dried over Na₂SO₄, filtered, and concentrated. The residue was purified by column chromatography (SiO₂, EtOAc/PET) to afford N⁴-(2,4-dimethoxybenzyl)-8-(3-methoxy-2,6-dimethylphenyl)-N⁶-methylpyrido[3,4-d]pyrimidine-4,6-diamine (1-3) (0.38 g, 35%). MS (ESI) m/z: 460.7 [M+H]⁺.

### 8-(3-methoxy-2,6-dimethylphenyl)-N⁶-methylpyrido[3,4-d]pyrimidine-4,6-diamine (1-4)

To a solution of N⁴-(2,4-dimethoxybenzyl)-8-(3-methoxy-2,6-dimethylphenyl)-N⁶-methylpyrido[3,4-d]pyrimidine-4,6-diamine (1-3) (0.37 g, 0.81 mmol) in CH₂Cl₂ (3.70 mL) were added TFA (0.92 mL, 12.08 mmol), and TfOH (0.71 mL, 8.05 mmol) at rt. The mixture was stirred at rt for 1 h. After completion, the reaction was quenched with sat. NaHCO₃ (20 mL) and extracted with EtOAc (2×30 mL). The combined organic layers were washed with brine (30 mL), dried over Na₂SO₄, filtered, and concentrated. The residue was purified by column chromatography (SiO₂, EtOAc/PET) to afford 8-(3-methoxy-2,6-dimethylphenyl)-N⁶-methylpyrido[3,4-d]pyrimidine-4,6-diamine (1-4) (0.18 g, 72%). MS (ESI) m/z: 310.3 [M+H]⁺.

### 3-(4-amino-6-(methylamino)pyrido[3,4-d]pyrimidin-8-yl)-2,4-dimethylphenol (1-5)

To a stirred solution of 8-(3-methoxy-2,6-dimethylphenyl)-N6-methylpyrido[3,4-d]pyrimidine-4,6-diamine (1-4) (0.18 g, 0.58 mmol) in CH₂Cl₂ (1.8 mL) was added BBr₃ (1M in DCM, 2.91 mL, 2.91 mmol) at 0° C. The mixture was stirred at rt for 30 min. After completion, the reaction was quenched with sat. NaHCO₃ (30 mL) and extracted with EtOAc (2×30 mL). The combined organic layers were washed with brine (30 mL), dried over Na₂SO₄, filtered, and concentrated. The residue was purified by Preparative HPLC (C18, ACN/H₂O with 10 mM NH₄HCO₃) to afford a racemic 3-(4-amino-6-(methylamino)pyrido[3,4-d]pyrimidin-8-yl)-2,4-dimethylphenol (1-A) (0.057 g, 33%). MS (ESI) 296.32 [M+H]⁺.

### Isolation of (S)-3-(4-amino-6-(methylamino)pyrido[3,4-d]pyrimidin-8-yl)-2,4-dimethylphenol (1-B) & (R)-3-(4-amino-6-(methylamino)pyrido[3,4-d]pyrimidin-8-yl)-2,4-dimethylphenol (1-C)

The racemic mixture of 3-(4-amino-6-(methylamino)pyrido[3,4-d]pyrimidin-8-yl)-2,4-dimethylphenol was subjected to chiral SFC purification (Chiralpak IJ, 30×250 mm column), 20% MeOH to afford the title compounds. From Peak 1: (S)-3-(4-amino-6-(methylamino)pyrido[3,4-d]pyrimidin-8-yl)-2,4-dimethylphenol (1-B) (19 mg, 33%). Chiral purity 99.7% (Chiralpak IJ (4.6×250 mm column), 20% MeOH with 0.5% DEA); ¹H NMR (400 MHz, DMSO-d₆): δ ppm 9.00 (s, 1H), 8.00 (s, 1H), 7.67 (br s, 2H), 6.84 (d, J=8.0 Hz, 1H), 6.78 (s, 1H), 6.71 (d, J=8.0 Hz, 1H), 6.58-6.55 (m, 1H), 2.81 (d, J=5.2 Hz, 3H), 1.71 (s, 3H), 1.63 (s, 3H). MS (ESI) m/z: 296.2 [M+H]⁺. From Peak 2: (R)-3-(4-amino-6-(methylamino)pyrido[3,4-d]pyrimidin-8-yl)-2,4-dimethylphenol (1-C) (21 mg, 36%). Chiral purity 99.5% (Chiralpak IJ (4.6×250 mm column), 20% MeOH with 0.5% DEA); ¹H NMR (400 MHz, DMSO-d₆): δ ppm 9.00 (s, 1H), 8.00 (s, 1H), 7.66 (br s, 2H), 6.84 (d, J=8.0 Hz, 1H), 6.78 (s, 1H), 6.71 (d, J=8.0 Hz, 1H), 6.57-6.55 (m, 1H), 2.81 (d, J=4.8 Hz, 3H), 1.71 (s, 3H), 1.63 (s, 3H). MS (ESI) m/z: 296.3 [M+H]⁺. Compounds 1-B and 1-C are atropisomers, and the shown stereochemistry may be the opposite of that depicted.

### Example 2

### (S)-3-(4-amino-6-(3,3-difluoropyrrolidin-1-yl)pyrido[3,4-d]pyrimidin-8-yl)-2,4-dimethylphenol (2-B) and (R)-3-(4-amino-6-(3,3-difluoropyrrolidin-1-yl)pyrido[3,4-d]pyrimidin-8-yl)-2,4-dimethylphenol (2-C)

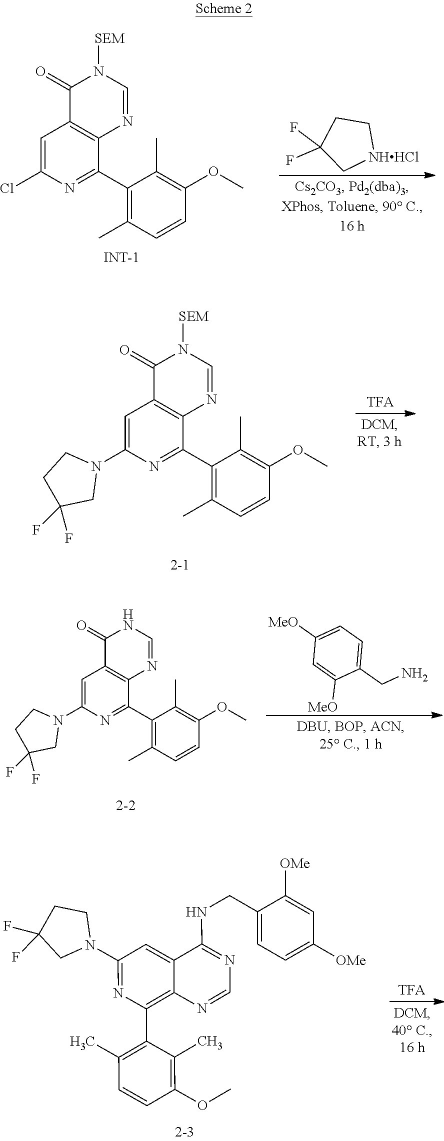

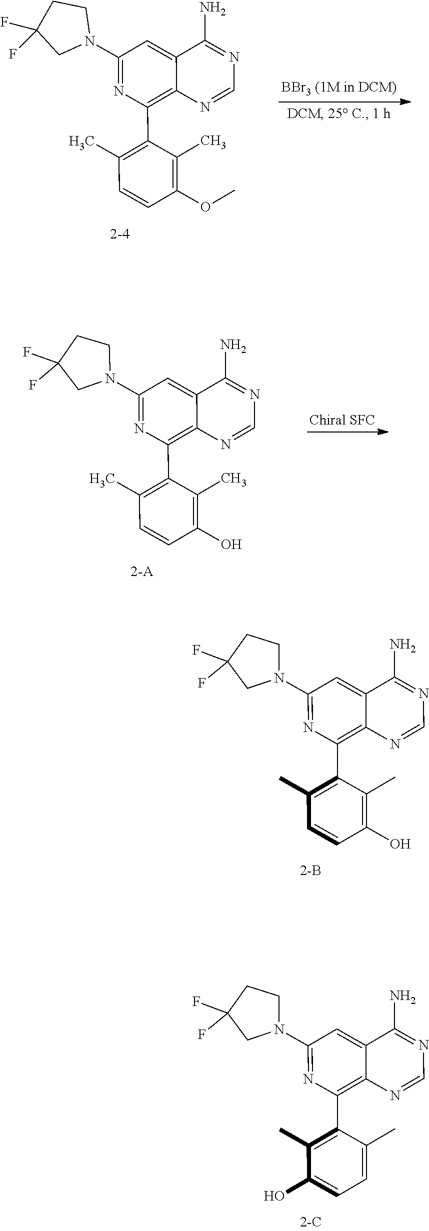

### 6-(3,3-difluoropyrrolidin-1-yl)-8-(3-methoxy-2,6-dimethylphenyl)-3-((2-(trimethylsilyl)ethoxy)methyl)pyrido[3,4-d]pyrimidin-4 (3H)-one (2-1)

To a solution of 6-chloro-8-(3-methoxy-2,6-dimethylphenyl)-3-((2-(trimethylsilyl)ethoxy)methyl)pyrido[3,4-d]pyrimidin-4 (3H)-one (INT-1) (0.50 g, 1.12 mmol) and 3,3-difluoropyrrolidine hydrochloride (0.12 g, 1.12 mmol) in toluene (5.0 mL) was added Cs₂CO₃ (1.83 g, 5.61 mmol) then degassed with N₂ for 15 min followed by addition of Pd₂dba₃ (0.10 g, 0.11 mmol) and Xphos (0.048 g, 0.10 mmol). The mixture was degassed again for another 10 min and heated at 90° C. for 16 h. After completion, water (30 mL) was added and extracted with EtOAc (2×30 mL). The combined organic layers were washed with brine (40 mL), dried over Na₂SO₄, filtered and concentrated to afford 6-(3,3-difluoropyrrolidin-1-yl)-8-(3-methoxy-2,6-dimethylphenyl)-3-((2-(trimethylsilyl)ethoxy)methyl)pyrido[3,4-d]pyrimidin-4 (3H)-one (2-1) (0.5 g, 87%). This material was taken on to the next step without further purification. MS (ESI) m/z: 517.5 [M+H]⁺.

### 6-(3,3-difluoropyrrolidin-1-yl)-8-(3-methoxy-2,6-dimethylphenyl)pyrido[3,4-d]pyrimidin-4 (3H)-one (2-2)

To a stirred solution of 6-(3,3-difluoropyrrolidin-1-yl)-8-(3-methoxy-2,6-dimethylphenyl)-3-((2-(trimethylsilyl)ethoxy)methyl)pyrido[3,4-d]pyrimidin-4 (3H)-one (2-1) (0.5 g, 0.97 mmol) in DCM (10.0 mL) was added TFA (1.58 mL, 19.36 mmol) at rt. The mixture was stirred at rt for 3 h. After completion, the reaction was quenched with sat. NaHCO₃ (10 mL) and extracted with EtOAc (3×30 mL). The combined organic layers were washed with brine (30 mL), dried over Na₂SO₄, filtered, and concentrated. The residue was purified by column chromatography (SiO₂, EtOAc/PET) to afford 6-(3,3-difluoropyrrolidin-1-yl)-8-(3-methoxy-2,6-dimethylphenyl)pyrido[3,4-d]pyrimidin-4 (3H)-one (2-2) (0.3 g, 80%). MS (ESI) m/z: 387.54 [M+H]⁺.

### 6-(3,3-difluoropyrrolidin-1-yl)-N-(2,4-dimethoxybenzyl)-8-(3-methoxy-2,6-dimethylphenyl)pyrido[3,4-d]pyrimidin-4-amine (2-3)

To a stirred solution of 6-(3,3-difluoropyrrolidin-1-yl)-8-(3-methoxy-2,6-dimethylphenyl)pyrido[3,4-d]pyrimidin-4 (3H)-one (2-2) (0.30 g, 0.78 mmol) and (2,4-dimethoxyphenyl)methanamine (0.26 g, 1.55 mmol) in ACN (6.00 mL) were added BOP (0.45 g, 1.01 mmol), DBU (0.17 mL, 1.17 mmol) at rt. The mixture was stirred at rt for 1 h. After completion, the reaction was quenched with water (30 mL) and extracted with EtOAc (3×30 mL). The combined organic layers were washed with brine (30 mL), dried over Na₂SO₄, filtered, and concentrated. The residue was purified by column chromatography (SiO₂, EtOAc/PET) to afford 6-(3,3-difluoropyrrolidin-1-yl)-N-(2,4-dimethoxybenzyl)-8-(3-methoxy-2,6-dimethylphenyl)pyrido[3,4-d]pyrimidin-4-amine (2-3) (0.3 g, 72%). MS (ESI) m/z: 536.7 [M+H]⁺.

### 6-(3,3-difluoropyrrolidin-1-yl)-8-(3-methoxy-2,6-dimethylphenyl)pyrido[3,4-d]pyrimidin-4-amine (2-4)

To a stirred solution of 6-(3,3-difluoropyrrolidin-1-yl)-N-(2,4-dimethoxybenzyl)-8-(3-methoxy-2,6-dimethylphenyl)pyrido[3,4-d]pyrimidin-4-amine (2-3) (0.30 g, 0.56 mmol,) in DCM (9.0 mL) was added TFA (0.43 mL, 5.60 mmol) at rt. The mixture was stirred at 40° C. for 16 h. After completion, the reaction was quenched with sat. NaHCO₃ (10 mL) and water (10 mL) was added, then extracted with EtOAc (2×20 mL). The combined organic layers were washed with brine (20 mL), dried over Na₂SO₄, filtered, and concentrated. The residue was purified by column chromatography (SiO₂, EtOAc/PET) to afford 6-(3,3-difluoropyrrolidin-1-yl)-8-(3-methoxy-2,6-dimethylphenyl)pyrido[3,4-d]pyrimidin-4-amine (2-4) (0.1 g, 46%). MS (ESI) m/z: 386.24 [M+H]⁺.

### 3-(4-amino-6-(3,3-difluoropyrrolidin-1-yl)pyrido[3,4-d]pyrimidin-8-yl)-2,4-dimethylphenol (2-5)

To a stirred solution of 6-(3,3-difluoropyrrolidin-1-yl)-8-(3-methoxy-2,6-dimethylphenyl)pyrido[3,4-d]pyrimidin-4-amine (2-4) (0.10 g, 0.26 mmol) in DCM (1.0 mL) was added BBr₃ (1M in DCM, 1.04 mL, 1.04 mmol) at 0° C. and the mixture was stirred at rt for 1 h. After completion, the mixture was evaporated under vacuum. Ice water was added to the mixture, stirred for 30 min., and then neutralized with aqueous NaHCO₃ (~5 mL). The solution was extracted with EtOAc (2×20 mL). The combined organic layers were washed with brine (10 mL), dried over Na₂SO₄, filtered, and concentrated. The residue was purified by column chromatography (SiO₂, EtOAc/PET) to afford 3-(4-amino-6-(3,3-difluoropyrrolidin-1-yl)pyrido[3,4-d]pyrimidin-8-yl)-2,4-dimethylphenol (2-A) (0.04 g, 41%). MS (ESI) m/z: 372.5 [M+H]⁺.

### Isolation of (S)-3-(4-amino-6-(3,3-difluoropyrrolidin-1-yl)pyrido[3,4-d]pyrimidin-8-yl)-2,4-dimethylphenol (2-B) and (R)-3-(4-amino-6-(3,3-difluoropyrrolidin-1-yl)pyrido[3,4-d]pyrimidin-8-yl)-2,4-dimethylphenol (2-C)

The racemic mixture of 3-(4-amino-6-(3,3-difluoropyrrolidin-1-yl)pyrido[3,4-d]pyrimidin-8-yl)-2,4-dimethylphenol was subjected to chiral SFC purification (ChiralCEL-OX—H, 30×250 mm column, 20% MeOH with 0.1% DEA) to afford the title compounds. From Peak 1: (S)-3-(4-amino-6-(3,3-difluoropyrrolidin-1-yl)pyrido[3,4-d]pyrimidin-8-yl)-2,4-dimethylphenol (2-B) (60 mg, 15%). Chiral purity 99.92% (Chiralpak OX—H, 4.6×250 mm column), 20% MeOH); ¹H-NMR (400 MHz, DMSO-d₆): δ ppm 9.05 (s, 1H), 8.07 (s, 1H), 7.79 (br. s, 2H), 7.04 (s, 1H), 6.86 (d, J=8.4 Hz, 1H), 6.74 (d, J=8 Hz, 1H), 3.85 (t, J=13.2 Hz, 2H), 3.66 (t, J=7.2 Hz, 2H), 2.63-2.55 (m, 2H), 1.72 (s, 3H), 1.63 (s, 3H). MS (ESI) m/z: 372.4 [M+H]⁺. From Peak 2: (R)-3-(4-amino-6-(3,3-difluoropyrrolidin-1-yl)pyrido[3,4-d]pyrimidin-8-yl)-2,4-dimethylphenol (2-C) (63 mg, 15%). Chiral purity 98.0% (Chiralpak OX—H, (4.6×250 mm column), 20% MeOH); ¹H-NMR (400 MHz, DMSO-d₆): δ ppm 9.05 (s, 1H), 8.07 (s, 1H), 7.79 (br. s, 2H), 7.04 (s, 1H), 6.86 (d, J=8.4 Hz, 1H), 6.74 (d, J=8 Hz, 1H), 3.85 (t, J=13.2 Hz, 2H), 3.66 (t, J=7.4 Hz, 2H), 2.63-2.55 (m, 2H), 1.72 (s, 3H), 1.63 (s, 3H). MS (ESI) m/z: 372.4 [M+H]⁺. Compounds 2-B and 2-C are atropisomers, and the shown stereochemistry may be the opposite of that depicted.

### Example 3

### (R)-1-((S)-4-amino-8-(3-hydroxy-2,6-dimethylphenyl)pyrido[3,4-d]pyrimidin-6-yl)pyrrolidin-3-ol (3-B) and (R)-1-((R)-4-amino-8-(3-hydroxy-2,6-dimethylphenyl)pyrido[3,4-d]pyrimidin-6-yl)pyrrolidin-3-ol (3-C)

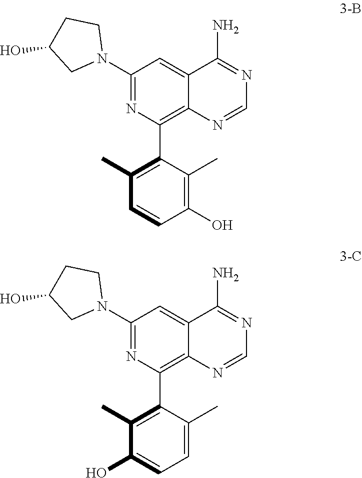

The racemic mixture (3R)-1-(4-amino-8-(3-hydroxy-2,6-dimethylphenyl)pyrido[3,4-d]pyrimidin-6-yl)pyrrolidin-3-ol (3-A) (0.09 g) was prepared following the procedure described in Example 1 and was subjected to chiral SFC purification (Chiralpak IJ, 30×250 mm column, 20% MeOH with 0.2% DEA) to afford the title compounds. From Peak 1: (R)-1-((S)-4-amino-8-(3-hydroxy-2,6-dimethylphenyl)pyrido[3,4-d]pyrimidin-6-yl)pyrrolidin-3-ol (3-B) (27 mg, 30%). Chiral purity 99.9% (Chiralpak IJ, 4.6×250 mm column, 20% MeOH with 0.5% DEA); ¹H NMR (400 MHz, DMSO-d₆): δ ppm 9.02 (s, 1H), 8.00 (s, 1H), 7.70 (br s, 2H), 6.86-6.84 (m, 2H), 6.73 (d, J=8.0 Hz, 1H), 4.98 (d, J=4.0 Hz, 1H), 4.44-4.35 (m, 1H), 3.53-3.47 (m, 3H), 3.35-3.34 (m, 1H), 2.09-2.03 (m, 1H), 1.93-1.91 (m, 1H), 1.73 (s, 3H), 1.64 (s, 3H). MS (ESI) m/z: 352.2 [M+H]⁺. From Peak 2: (R)-1-((R)-4-amino-8-(3-hydroxy-2,6-dimethylphenyl)pyrido[3,4-d]pyrimidin-6-yl)pyrrolidin-3-ol (3-C) (37 mg, 41%). Chiral purity 97.8% (Chiralpak IJ, 4.6×250 mm column, 20% MeOH with 0.5% DEA); ¹H NMR (400 MHz, DMSO-d₆): δ ppm 9.02 (s, 1H), 8.00 (s, 1H), 7.70 (br s, 2H), 6.86-6.84 (m, 2H), 6.73 (d, J=8.4 Hz, 1H), 4.98 (d, J=3.6 Hz, 1H), 4.45-4.35 (m, 1H), 3.53-3.47 (m, 3H), 3.35-3.32 (m, 1H), 2.11-2.02 (m, 1H), 1.94-1.92 (m, 1H), 1.73 (s, 3H), 1.64 (s, 3H). MS (ESI) m/z: 352.2 [M+H]⁺. Compounds 3-B and 3-C are atropisomers, and the shown stereochemistry may be the opposite of that depicted.

### Example 4

### (S)-1-((S)-4-amino-8-(3-hydroxy-2,6-dimethylphenyl)pyrido[3,4-d]pyrimidin-6-yl)pyrrolidin-3-ol (4-B) and (S)-1-((R)-4-amino-8-(3-hydroxy-2,6-dimethylphenyl) pyrido[3,4-d]pyrimidin-6-yl)pyrrolidin-3-ol (4-C)

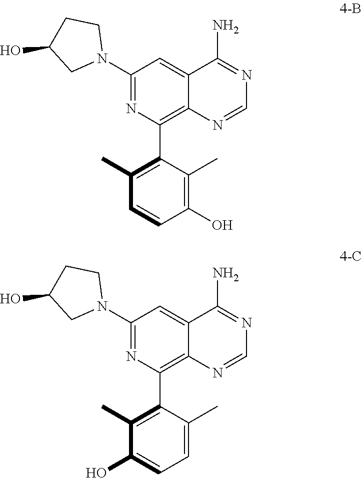

The racemic mixture of (3S)-1-(4-amino-8-(3-hydroxy-2,6-dimethylphenyl)pyrido[3,4-d]pyrimidin-6-yl)pyrrolidin-3-ol (4-A) (0.08 g) was prepared following the procedure described in Example 1 and was subjected to chiral SFC purification (Lux i-Amylose-3, 30×250 mm column, 23% ACN/MeOH (1:1) with 0.1% 7N NH₃ in MeOH) to afford the title compounds. From Peak 1: (S)-1-((S)-4-amino-8-(3-hydroxy-2,6-dimethylphenyl)pyrido[3,4-d]pyrimidin-6-yl)pyrrolidin-3-ol (4-B) (12.05 mg, 15.0%). Chiral purity 99.8% (Lux-i-Amylose-3, 4.6×250 mm column, 25% of ACN/MeOH (1:1) with 0.2% 7N NH₃ in MeOH). ¹H NMR (400 MHz, DMSO-d₆): δ ppm 9.04 (s, 1H), 8.01 (s, 1H), 7.74 (br. s, 2H), 6.87-6.84 (m, 2H), 6.74 (d, J=8.0 Hz, 1H), 4.98 (d, J=3.6 Hz, 1H), 4.47-4.43 (m, 1H), 3.54-3.47 (m, 3H), 3.34-3.31 (m, 1H), 2.12-2.05 (m, 1H), 1.96-1.87 (m, 1H), 1.73 (s, 3H), 1.64 (s, 3H). MS (ESI) m/z: 352.2 [M+H]⁺. From Peak 2: (S)-1-((R)-4-amino-8-(3-hydroxy-2,6-dimethylphenyl)pyrido[3,4-d]pyrimidin-6-yl)pyrrolidin-3-ol (4-C) (21.62 mg, 26.3%). Chiral purity 99.0% (Lux-i-Amylose-3, 4.6×250 mm column, 25% of ACN/MeOH (1:1) with 0.2% 7N NH₃ in MeOH); ¹H NMR (400 MHz, DMSO-d₆): δ ppm 9.02 (s, 1H), 8.00 (s, 1H), 7.71 (br. s, 2H), 6.86-6.83 (m, 2H), 6.73 (d, J=8.0 Hz, 1H), 4.98 (d, J=3.6 Hz, 1H), 4.46-4.40 (m, 1H), 3.53-3.46 (m, 3H), 3.33 (s, 1H), 2.12-2.05 (m, 1H), 1.96-1.87 (m, 1H), 1.73 (s, 3H), 1.64 (s, 3H). MS (ESI) m/z: 352.4 [M+H]⁺. Compounds 4-B and 4-C are atropisomers, and the shown stereochemistry may be the opposite of that depicted.

### Example 5

### (S)-3-(4-Amino-6-(3,3-difluoroazetidin-1-yl)pyrido[3,4-d]pyrimidin-8-yl)-2,4-dimethylphenol (5-B) and (R)-3-(4-amino-6-(3,3-difluoroazetidin-1-yl)pyrido[3,4-d]pyrimidin-8-yl)-2,4-dimethylphenol (5-C)

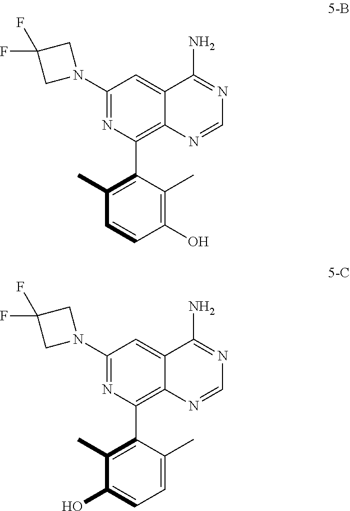

The racemic mixture of 3-(4-amino-6-(3,3-difluoroazetidin-1-yl)pyrido[3,4-d]pyrimidin-8-yl)-2,4-dimethylphenol (5-A) (80 mg) was prepared following the procedure described in Example 2 (replacing Cs₂CO₃ with NaO^{t}Bu and Xphos with Xantphos in 1,4-dioxane in the first step) and was subjected to chiral SFC purification (Chiralpak IJ, 30×250 mm column, 20% MeOH) to afford the title compounds. From Peak 1: (S)-3-(4-amino-6-(3,3-difluoroazetidin-1-yl)pyrido[3,4-d]pyrimidin-8-yl)-2,4-dimethylphenol (5-B) (19.97 mg, 33%). Chiral purity 99.8% (Chiralpak IJ (4.6×250 mm column), 20% MeOH); ¹H NMR (400 MHz, DMSO-d₆): δ ppm 9.11 (s, 1H), 8.13 (s, 1H), 7.86 (br. s, 2H), 7.19 (s, 1H), 6.85 (d, J=8.4 Hz, 1H), 6.74 (d, J=8.0 Hz, 1H), 4.40 (t, J=12.2 Hz, 4H), 1.71 (s, 3H), 1.62 (s, 3H). MS (ESI) m/z: 358.3 [M+H]⁺. From Peak 2: (R)-3-(4-amino-6-(3,3-difluoroazetidin-1-yl)pyrido[3,4-d]pyrimidin-8-yl)-2,4-dimethylphenol (5-C) (26.42 mg, 44%). Chiral purity 99.5% (Chiralpak IJ (4.6×250 mm column), 20% MeOH); ¹H NMR (400 MHz, DMSO-d₆): δ ppm 9.12 (s, 1H), 8.17 (br. s, 3H), 7.23 (s, 1H), 6.88 (d, J=8.4 Hz, 1H), 6.77 (d, J=8.0 Hz, 1H), 4.42 (t, J=12.2 Hz, 4H), 1.72 (s, 3H), 1.64 (s, 3H). MS (ESI) m/z: 358.3 [M+H]⁺. Compounds 5-B and 5-C are atropisomers, and the shown stereochemistry may be the opposite of that depicted.

### Example 6

### (S)-3-(4-Amino-6-(pyrrolidin-1-yl)pyrido[3,4-d]pyrimidin-8-yl)-2,4-dimethylphenol (6-B) and (R)-3-(4-amino-6-(pyrrolidin-1-yl)pyrido[3,4-d]pyrimidin-8-yl)-2,4-dimethylphenol (6-C)

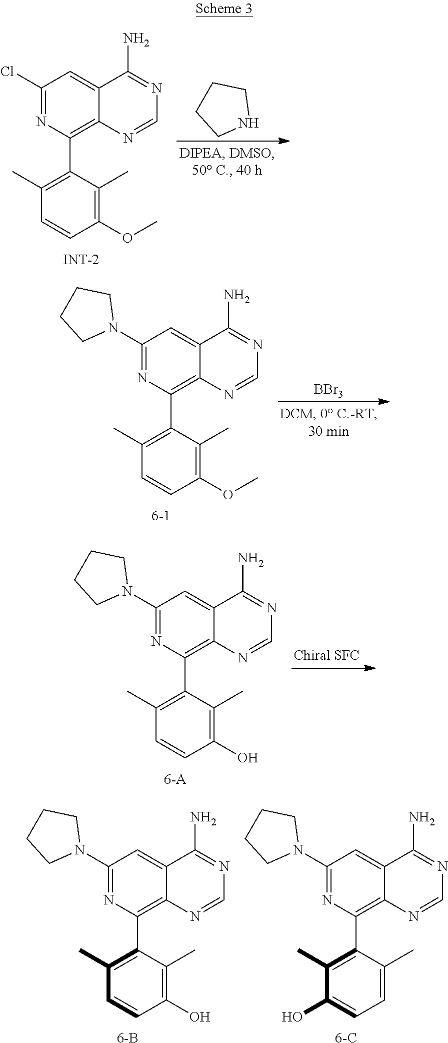

### 8-(3-methoxy-2,6-dimethylphenyl)-6-(pyrrolidin-1-yl)pyrido[3,4-d]pyrimidin-4-amine (6-1)

To a stirred solution of 6-chloro-8-(3-methoxy-2,6-dimethylphenyl)pyrido[3,4-d]pyrimidin-4-amine (INT-2) (0.15 g, 0.48 mmol) in DMSO (1.50 mL) were added pyrrolidine (0.10 g, 1.43 mmol) and DIPEA (0.19 g, 1.43 mmol) at rt. The mixture was heated at 50° C. for 40 h. The mixture was diluted with ice cold water (80 mL). The product was collected and dried under vacuum for 1 h to afford 8-(3-methoxy-2,6-dimethylphenyl)-6-(pyrrolidin-1-yl)pyrido[3,4-d]pyrimidin-4-amine (6-1) (0.15 g, 89.5%). MS (ESI) m/z: 350.2 [M+H]⁺.

### 3-(4-amino-6-(pyrrolidin-1-yl)pyrido[3,4-d]pyrimidin-8-yl)-2,4-dimethylphenol (6-2)

To a stirred solution of 8-(3-methoxy-2,6-dimethylphenyl)-6-(pyrrolidin-1-yl)pyrido[3,4-d]pyrimidin-4-amine (6-1) (0.14 g, 0.40 mmol) in CH₂Cl₂ (1.4 mL) at 0° C. was added BBr₃ (1M CH₂Cl₂, 1.20 mL, 1.20 mmol). The mixture was stirred at rt for 15 min., then diluted with CH₂Cl₂ (100 mL) and basified with sat. NaHCO₃ to pH~8. The organic layer with water (2×50 mL), dried over MgSO₄, filtered, and concentrated. The residue was purified by prep-HPLC (XSELECT C18, ACN/H₂O with 10 mM NH₄HCO₃) to afford a racemic 3-(4-amino-6-(pyrrolidin-1-yl)pyrido[3,4-d]pyrimidin-8-yl)-2,4-dimethylphenol (6-2) (0.06 g, 46%). MS (ESI) m/z: 336.40 [M+H]⁺.

### (S)-3-(4-amino-6-(pyrrolidin-1-yl)pyrido[3,4-d]pyrimidin-8-yl)-2,4-dimethylphenol (6-3) and (R)-3-(4-amino-6-(pyrrolidin-1-yl)pyrido[3,4-d]pyrimidin-8-yl)-2,4-dimethylphenol (6-4)

The racemic mixture of 3-(4-amino-6-(pyrrolidin-1-yl)pyrido[3,4-d]pyrimidin-8-yl)-2,4-dimethylphenol (6-A) (60 mg) was subjected to chiral SFC purification (Chiralpak IC, 30×250 mm column, 30% ⁱPrOH with 0.2% PrNH₂) then repurified by achiral SFC (DCPAK P4VP, 30×250 mm column, 30% MeOH) to afford the title compounds. From Peak 1: (S)-3-(4-amino-6-(pyrrolidin-1-yl)pyrido[3,4-d]pyrimidin-8-yl)-2,4-dimethylphenol (6-B) (20 mg, 30%). Chiral purity 99.4% (Lux i-Amylose-3 (4.6×250 mm column), 25% ACN/IPA (1:1) with 0.2% DEA); ¹H NMR (400 MHz, DMSO-d₆): δ ppm 12.94 (br. s, 1H), 9.81 (br. s, 1H), 9.36 (s, 1H), 8.23 (s, 1H), 7.23-6.96 (m, 2H), 6.86 (d, J=8.0 Hz, 1H), 3.49-3.42 (m, 4H), 2.06-1.95 (m, 4H), 1.79 (s, 3H), 1.70 (s, 3H). MS (ESI) m/z: 336.4 [M+H]⁺. From Peak 2: (R)-3-(4-amino-6-(pyrrolidin-1-yl)pyrido[3,4-d]pyrimidin-8-yl)-2,4-dimethylphenol (6-C) (18 mg, 30%). Chiral purity 98.0% (Lux i-Amylose (4.6×250 mm column), 25% ACN/ⁱPrOH (1:1) with 0.2% PrNH₂); ¹H NMR (400 MHz, DMSO-d₆): δ ppm 9.18-8.10 (m, 3H), 7.00 (br. s, 1H), 6.91 (d, J=8.0 Hz, 1H), 6.80 (d, J=8.0 Hz, 1H), 3.46-3.40 (m, 4H), 2.03-1.96 (m, 4H), 1.76 (s, 3H), 1.67 (s, 3H). MS (ESI) m/z: 336.4 [M+H]⁺. Compounds 6-B and 6-C are atropisomers, and the shown stereochemistry may be the opposite of that depicted.

### Example 7

### (S)-3-(4-Amino-6-morpholinopyrido[3,4-d]pyrimidin-8-yl)-2,4-dimethylphenol (7-B) and (R)-3-(4-amino-6-morpholinopyrido[3,4-d]pyrimidin-8-yl)-2,4-dimethylphenol (7-C)

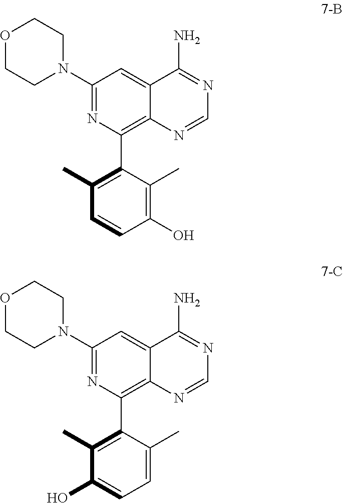

The racemic mixture of 3-(4-amino-6-morpholinopyrido[3,4-d]pyrimidin-8-yl)-2,4-dimethylphenol (7-A) (0.14 g) was prepared following the procedure described in Example 1 and was subjected to chiral SFC purification (DCPAK P4VP, 30×250 mm column, 25% MeOH) to afford the title compounds. From Peak 1: (S)-3-(4-amino-6-morpholinopyrido[3,4-d]pyrimidin-8-yl)-2,4-dimethylphenol (7-B) (60 mg, 33%). Chiral purity 98.9% (Lux Cellulose-4 (4.6×150 mm column), 20% MeOH with 0.5% DEA); ¹H NMR (400 MHz, DMSO-d₆): δ ppm 9.04 (br. s, 1H), 8.09 (s, 1H), 7.81 (br. s, 2H), 7.30 (s, 1H), 6.85 (d, J=8 Hz, 1H), 6.73 (d, J=8 Hz, 1H), 3.78-3.73 (m, 4H), 3.50-3.44 (m, 4H), 1.72 (s, 3H), 1.63 (s, 3H); MS (ESI) m/z: 352.5 [M+H]⁺. From Peak 2: (R)-3-(4-amino-6-morpholinopyrido[3,4-d]pyrimidin-8-yl)-2,4-dimethylphenol (7-C) (72 mg, 40%). Chiral purity 97.2% (Lux Cellulose-4 (4.6×150 mm column), 20% MeOH with 0.5% DEA); ¹H NMR (400 MHz, DMSO-d₆): δ ppm 9.04 (s, 1H), 8.09 (s, 1H), 7.81 (br. s, 2H), 7.30 (s, 1H), 6.85 (d, J=8 Hz, 1H), 6.73 (d, J=8 Hz, 1H), 3.78-3.73 (m, 4H), 3.50-3.44 (m, 4H), 1.72 (s, 3H), 1.63 (s, 3H); MS (ESI) m/z: 352.5 [M+H]⁺. Compounds 7-B and 7-C are atropisomers, and the shown stereochemistry may be the opposite of that depicted.

### Example 8

### (S)-((4-amino-8-(3-hydroxy-2,6-dimethylphenyl)pyrido[3,4-d]pyrimidin-6-yl)imino)dimethyl-λ⁶-sulfanone (8-B) and (R)-((4-amino-8-(3-hydroxy-2,6-dimethylphenyl)pyrido[3,4-d]pyrimidin-6-yl)imino)dimethyl-λ⁶-sulfanone (8-C)

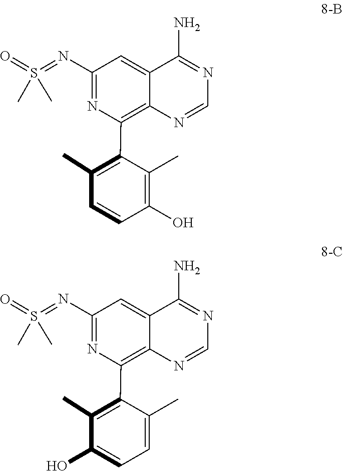

The racemic mixture of ((4-amino-8-(3-hydroxy-2,6-dimethylphenyl)pyrido[3,4-d]pyrimidin-6-yl)imino)dimethyl-X⁶-sulfanone (8-A) (0.08 g) was prepared following the procedure in Example 2 (replacing Cs₂CO₃ with NaOtBu in 1,4-dioxane/toluene in the first step) and was subjected to chiral SFC purification (Chiralpak IJ, 30×250 mm column, 20% MeOH) to afford the title compounds. From Peak 1: (S)-((4-amino-8-(3-hydroxy-2,6-dimethylphenyl)pyrido[3,4-d]pyrimidin-6-yl)imino)dimethyl-λ⁶-sulfanone (8-B) (11 mg, 13%). Chiral purity 99.4% (Chiralpak IJ (4.6×250 mm column), 20% MeOH with 0.5% DEA); ¹H NMR (400 MHz, DMSO-d₆): δ ppm 9.07 (br. s, 1H), 8.11 (s, 1H), 7.80 (br s, 2H), 7.34 (s, 1H), 6.87 (d, J=8.0 Hz, 1H), 6.74 (d, J=8.0 Hz, 1H), 3.33 (s, 6H), 1.73 (s, 3H), 1.64 (s, 3H). MS (ESI) m/z: 358.2 [M+H]⁺. From Peak 2, (R)-((4-amino-8-(3-hydroxy-2,6-dimethylphenyl)pyrido[3,4-d]pyrimidin-6-yl)imino)dimethyl-λ⁶-sulfanone, (8-C) (12 mg, 15%). Chiral purity 98.7% (Chiralpak IJ (4.6×250 mm column), 20% MeOH with 0.5% DEA); ¹H NMR (400 MHz, DMSO-d₆): δ ppm 9.07 (br. s, 1H), 8.11 (s, 1H), 7.80 (br s, 2H), 7.34 (s, 1H), 6.87 (d, J=8.4 Hz, 1H), 6.74 (d, J=8.0 Hz, 1H), 3.32 (s, 6H), 1.73 (s, 3H), 1.64 (s, 3H); MS (ESI) m/z: 358.3 [M+H]⁺. Compounds 8-B and 8-C are atropisomers, and the shown stereochemistry may be the opposite of that depicted.

### Example 9

### (S)-3-(4-Amino-6-((2-methoxyethyl)(methyl)amino)pyrido[3,4-d]pyrimidin-8-yl)-2,4-dimethylphenol (9-B) and (R)-3-(4-amino-6-((2-methoxyethyl)(methyl)amino)pyrido[3,4-d]pyrimidin-8-yl)-2,4-dimethylphenol (9-C)

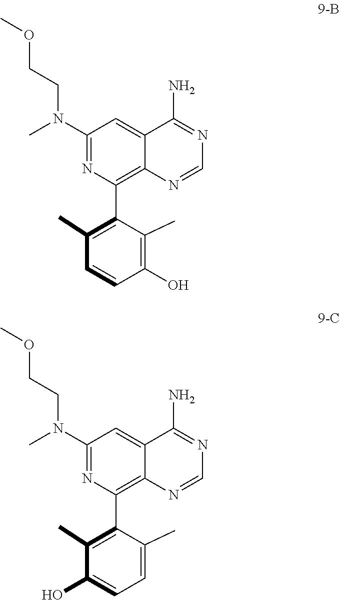

The racemic mixture of 3-(4-amino-6-((2-methoxyethyl)(methyl)amino)pyrido[3,4-d]pyrimidin-8-yl)-2,4-dimethylphenol (9-A) (0.06 g) was prepared following the procedure described in Example 1 and was subjected to chiral SFC purification (Chiralpak ADH (30×250 mm column, 20% ⁱPrOH with 0.1% ⁱPrNH₂) to afford the title compounds: From Peak 1: (S)-3-(4-amino-6-((2-methoxyethyl)(methyl)amino)pyrido[3,4-d]pyrimidin-8-yl)-2,4-dimethylphenol (9-B) (11 mg, 14%). Chiral purity 99.2% (Chiralpak AD-H (4.6×250 mm column), 20% PrOH with 0.5% PrNH₂); ¹H NMR (400 MHz, DMSO-d₆): δ ppm 9.03 (br. s, 1H), 8.02 (s, 1H), 7.74 (br. s, 2H), 7.00 (s, 1H), 6.85 (d, J=8.4 Hz, 1H), 6.73 (d, J=8.0 Hz, 1H), 3.75 (t, J=5.8 Hz, 2H), 3.49 (t, J=5.8 Hz, 2H), 3.22 (s, 3H), 3.08 (s, 3H), 1.72 (s, 3H), 1.63 (s, 3H). MS (ESI) m/z: 354.4 [M+H]⁺. From Peak 2: (R)-3-(4-amino-6-((2-methoxyethyl) (methyl)amino)pyrido[3,4-d]pyrimidin-8-yl)-2,4-dimethylphenol (9-C) (11 mg, 14%). Chiral purity 98.8% (Chiralpak AD-H (4.6×250 mm column), 20% ⁱPrOH with 0.5% PrNH₂); ¹H NMR (400 MHz, DMSO-d₆): δ ppm 9.03 (br. s, 1H), 8.02 (s, 1H), 7.74 (br. s, 2H), 7.00 (s, 1H), 6.85 (d, J=8.0 Hz, 1H), 6.73 (d, J=8.4 Hz, 1H), 3.75 (t, J=5.8 Hz, 2H), 3.49 (t, J=5.8 Hz, 2H), 3.22 (s, 3H), 3.08 (s, 3H), 1.72 (s, 3H), 1.63 (s, 3H); MS (ESI) m/z: 354.4 [M+H]⁺. Compounds 9-B and 9-C are atropisomers, and the shown stereochemistry may be the opposite of that depicted.

### Example 10

### (S)-3-(4-Amino-6-(dimethylamino)pyrido[3,4-d]pyrimidin-8-yl)-2,4-dimethylphenol (10-B) and (R)-3-(4-amino-6-(dimethylamino)pyrido[3,4-d]pyrimidin-8-yl)-2,4-dimethylphenol (10-C)

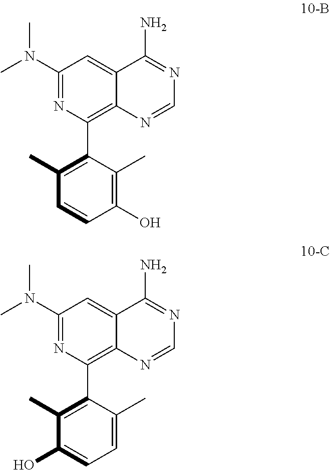

The racemic mixture of 3-(4-amino-6-(dimethylamino)pyrido[3,4-d]pyrimidin-8-yl)-2,4-dimethylphenol (10-A) (0.05 g) was prepared following the procedure described in Example 1 and was subjected to chiral SFC (Chiralpak IJ, 30×250 mm column, 20% MeOH with 0.1% DEA in MeOH) to afford the title compounds. From Peak 1: (S)-3-(4-amino-6-(dimethylamino)pyrido[3,4-d]pyrimidin-8-yl)-2,4-dimethylphenol (10-B) (18 mg, 15%). Chiral purity: 99.90% (Chiralpak IJ, 4.6×250 mm column, 20% MeOH with 0.5% DEA). ¹H-NMR (400 MHz, DMSO-d₆): δ 9.02 ppm (s, 1H), 8.02 (s, 1H), 7.74 (br. s, 2H), 7.04 (s, 1H), 6.85 (d, J=8 Hz, 1H), 6.73 (d, J=8.4 Hz, 1H), 3.07 (s, 6H), 1.72 (s, 3H), 1.63 (s, 3H). MS (ESI): 310.4 [M+H]⁺. From Peak 2: (R)-3-(4-amino-6-(dimethylamino)pyrido[3,4-d]pyrimidin-8-yl)-2,4-dimethylphenol (10-C) (21 mg, 17%). Chiral purity: 96.84% (Chiralpak IJ, 4.6×250 mm column, 20% MeOH with 0.5% DEA). ¹H-NMR (400 MHz, DMSO-d₆): δ ppm 9.02 (s, 1H), 8.02 (br. s, 1H), 7.74 (br. s, 2H), 7.04 (s, 1H), 6.85 (d, J=8 Hz, 1H), 6.73 (d, J=8.0 Hz, 1H), 3.07 (s, 6H), 1.72 (s, 3H), 1.63 (s, 3H). MS (ESI): 310.4 [M+H]⁺. Compounds 10-B and 10-C are atropisomers, and the shown stereochemistry may be the opposite of that depicted.

### Example 11

### (S)-1-(4-Amino-8-(3-hydroxy-2,6-dimethylphenyl)pyrido[3,4-d]pyrimidin-6-yl)piperidin-4-ol (11-B) and (R)-1-(4-amino-8-(3-hydroxy-2,6-dimethylphenyl)pyrido[3,4-d]pyrimidin-6-yl) piperidin-4-ol (11-C)

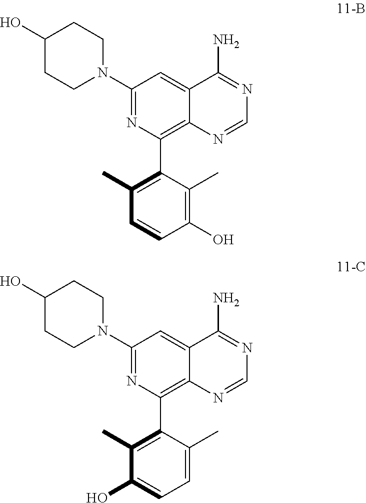

The racemic mixture of 1-(4-amino-8-(3-hydroxy-2,6-dimethylphenyl)pyrido[3,4-d]pyrimidin-6-yl)piperidin-4-ol (11-A) (0.03 g) was prepared following the procedure described in Example 1 and was subjected to chiral SFC purification (LUX CELLULOSE-4, 330×250 mm column, 30% ACN/H₂O (1:1) with 10 mM NH₄HCO₃) to afford the title compounds. From Peak 1: (S)-1-(4-amino-8-(3-hydroxy-2,6-dimethylphenyl)pyrido[3,4-d]pyrimidin-6-yl)piperidin-4-ol (11-B) (7.2 mg, 14%). Chiral purity 99.3% (Chiralcel OX-3 (4.6×150 mm column), 30% ACN/H₂O (1:1) with 0.2% NH₃ in MeOH); ¹H NMR (400 MHz, DMSO-d₆): δ ppm 9.06 (br. s, 1H), 8.07 (s, 1H), 7.83 (br. s, 2H), 7.30 (s, 1H), 6.86 (d, J=8.0 Hz, 1H), 6.75 (d, J=8.0 Hz, 1H), 4.69 (s 1H), 4.06-4.02 (m, 2H), 3.72 (br. s, 1H), 3.16-3.10 (m, 2H), 1.81-1.79 (m, 2H), 1.73 (s, 3H), 1.64 (s, 3H), 1.43-1.35 (m, 2H); MS (ESI) m/z: 366.5 [M+H]⁺. From Peak 2: (R)-1-(4-amino-8-(3-hydroxy-2,6-dimethylphenyl)pyrido[3,4-d]pyrimidin-6-yl)piperidin-4-ol (11-C) (10 mg, 20%). Chiral purity 94.1% (Chiralcel OX-3 (4.6×150 mm column), 30% ACN/H₂O (1:1) with 0.2% NH₃ in MeOH); ¹H NMR (400 MHz, DMSO-d₆): δ ppm 9.04 (br. s, 1H), 8.05 (s, 1H), 7.75 (br. s, 2H), 7.27 (s, 1H), 6.85 (d, J=8.0 Hz, 1H), 6.73 (d, J=8.0 Hz, 1H), 4.69 (br. s, 1H), 4.06-4.02 (m, 2H), 3.71 (br. s, 1H), 3.15-3.09 (m, 2H), 1.81-1.78 (m, 2H), 1.72 (s, 3H), 1.63 (s, 3H), 1.43-1.36 (m, 2H). MS (ESI) m/z: 366.4 [M+H]⁺. Compounds 11-B and 11-C are atropisomers, and the shown stereochemistry may be the opposite of that depicted.

### Example 12

### (S)-3-(4-Amino-6-(isopropyl(methyl)amino)pyrido[3,4-d]pyrimidin-8-yl)-2,4-dimethylphenol (12-B) and (R)-3-(4-amino-6-(isopropyl(methyl)amino)pyrido[3,4-d]pyrimidin-8-yl)-2,4-dimethylphenol (12-C)

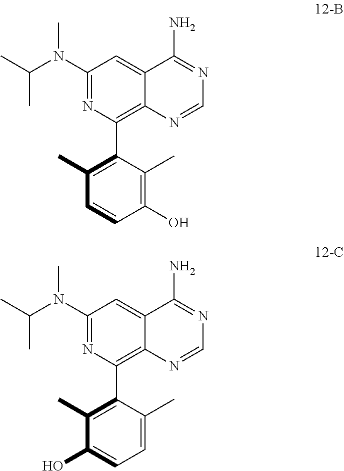

The racemic mixture of 3-(4-amino-6-(isopropyl(methyl)amino)pyrido[3,4-d]pyrimidin-8-yl)-2,4-dimethylphenol (12-A) (0.10 g) was prepared following the procedure described in Example 1 and was subjected to chiral SFC purification (Chiralpak IC, 30×250 mm column, 20% IPA with 0.1% PrNH₂) to afford the title compounds. From Peak 1: (S)-3-(4-amino-6-(isopropyl(methyl)amino)pyrido[3,4-d]pyrimidin-8-yl)-2,4-dimethylphenol (12-B) (21 mg, 21%). Chiral purity 98.6% (Chiralpak IC (4.6×250 mm column), 20% IPA with 0.2% PrNH₂); ¹H NMR (400 MHz, DMSO-d₆): δ ppm 9.02 (s, 1H), 8.01 (s, 1H), 7.72 (br. s, 2H), 7.00 (s, 1H), 6.85 (d, J=8.0 Hz, 1H), 6.73 (d, J=8.4 Hz, 1H), 4.97-4.88 (m, 1H), 2.86 (s, 3H), 1.73 (s, 3H), 1.64 (s, 3H), 1.12 (d, J=6.4 Hz, 6H); MS (ESI) m/z: 338.3 [M+H]⁺. From Peak 2: (R)-3-(4-amino-6-(isopropyl(methyl)amino)pyrido[3,4-d]pyrimidin-8-yl)-2,4-dimethylphenol (12-C) (25 mg, 25%). Chiral purity 98.4% (Chiralpak IC (4.6×250 mm column), 20% IPA with 0.2% ⁱPrNH₂); ¹H NMR (400 MHz, DMSO-d₆): δ ppm 9.03 (s, 1H), 8.02 (s, 1H), 7.73 (br. s, 2H), 7.00 (s, 1H), 6.85 (d, J=8.0 Hz, 1H), 6.73 (d, J=8.0 Hz, 1H), 4.98-4.88 (m, 1H), 2.85 (s, 3H), 1.73 (s, 3H), 1.64 (s, 3H), 1.11 (d, J=6.8 Hz, 6H). MS (ESI) m/z: 338.3 [M+H]⁺. Compounds 12-B and 12-C are atropisomers, and the shown stereochemistry may be the opposite of that depicted.

### Example 13

### (S)-1-((S)-4-Amino-8-(3-hydroxy-2,6-dimethylphenyl)pyrido[3,4-d]pyrimidin-6-yl)-3-methylpyrrolidin-3-ol (13-B), (R)-1-((S)-4-amino-8-(3-hydroxy-2,6-dimethylphenyl)pyrido[3,4-d]pyrimidin-6-yl)-3-methylpyrrolidin-3-ol (13-C), (S)-1-((R)-4-amino-8-(3-hydroxy-2,6-dimethylphenyl)pyrido[3,4-d]pyrimidin-6-yl)-3-methylpyrrolidin-3-ol (13-E) and (R)-1-((R)-4-amino-8-(3-hydroxy-2,6-dimethylphenyl)pyrido[3,4-d]pyrimidin-6-yl)-3-methylpyrrolidin-3-ol (13-F)

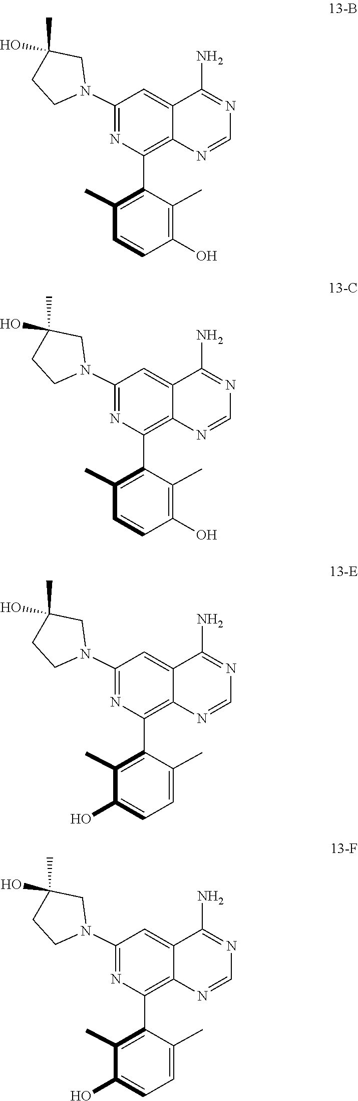

The racemic mixture of 1-(4-amino-8-(3-hydroxy-2,6-dimethylphenyl)pyrido[3,4-d]pyrimidin-6-yl)-3-methylpyrrolidin-3-ol (0.08 g) was prepared following the procedure described in Example 6 and was subjected to chiral SFC purification (Chiralpak-IH, 30×250 mm column, 45% ACN with 0.1% DEA) to afford Peak-1 (33 mg, 43%) and Peak-2 (29 mg, 38%).

Peak-1 (13-A) was further subjected to chiral SFC purification (Chiralpak IC, 30×250 mm column, 25% IPA with 0.1% ⁱPrNH₂) to afford the title compounds. From Peak 1A: (S)-1-((S)-4-amino-8-(3-hydroxy-2,6-dimethylphenyl)pyrido[3,4-d]pyrimidin-6-yl)-3-methylpyrrolidin-3-ol (13-B) (13 mg, 39%). Chiral purity 99.2% (Chiralpak IC (4.6×250 mm column), 25% IPA with 0.2% PrNH₂); ¹H NMR (400 MHz, DMSO-d₆): δ ppm 9.02 (s, 1H), 7.99 (s, 1H), 7.69 (br. s, 2H), 6.89-6.83 (m, 2H), 6.73 (d, J=8.0 Hz, 1H), 4.81 (s, 1H), 3.57-3.49 (m, 2H), 3.43-3.40 (m, 1H), 3.32-3.27 (m, 1H), 1.99-1.90 (m, 2H), 1.73 (s, 3H), 1.64 (s, 3H), 1.37 (s, 3H). MS (ESI) m/z: 366.3 [M+H]⁺. From Peak 1B: (R)-1-((S)-4-amino-8-(3-hydroxy-2,6-dimethylphenyl)pyrido[3,4-d]pyrimidin-6-yl)-3-methylpyrrolidin-3-ol (13-C) (12 mg, 36%). Chiral purity 96.2% (Chiralpak IC (4.6×250 mm column), 25% IPA with 0.2% PrNH₂); ¹H NMR (400 MHz, DMSO-d₆): δ ppm 9.02 (s, 1H), 7.99 (s, 1H), 7.69 (br. s, 2H), 6.86-6.81 (m, 2H), 6.73 (d, J=8.0 Hz, 1H), 4.81 (s, 1H), 3.53-3.50 (m, 2H), 3.43-3.40 (m, 1H), 3.30-3.27 (m, 1H), 1.96-1.90 (m, 2H), 1.73 (s, 3H), 1.64 (s, 3H), 1.37 (s, 3H). MS (ESI) m/z: 366.4 [M+H]⁺.

Peak-2 (13-D) (29 mg) was further subjected to chiral SFC purification (Chiralpak IC, 30×250 mm, 25% IPA with 0.2% PrNH₂) to afford the title compounds. From Peak 2A: (S)-1-((R)-4-amino-8-(3-hydroxy-2,6-dimethylphenyl)pyrido[3,4-d]pyrimidin-6-yl)-3-methylpyrrolidin-3-ol (13-E) (8 mg, 28%). Chiral purity 98.0% (Chiralpak IC (4.6×250 mm column), 25% IPA with 0.2% PrNH₂); ¹H NMR (400 MHz, DMSO-d₆): δ ppm 9.01 (s, 1H), 7.99 (s, 1H), 7.69 (br. s, 2H), 6.86-6.83 (m, 2H), 6.73 (d, J=8.0 Hz, 1H), 4.81 (s, 1H), 3.53-3.49 (m, 2H), 3.43-3.40 (m, 1H), 3.27-3.17 (m, 1H), 1.97-1.90 (m, 2H), 1.73 (s, 3H), 1.64 (s, 3H), 1.37 (s, 3H). MS (ESI) m/z: 366.3 [M+H]⁺. From Peak 2B: (R)-1-((R)-4-amino-8-(3-hydroxy-2,6-dimethylphenyl)pyrido[3,4-d]pyrimidin-6-yl)-3-methylpyrrolidin-3-ol (13-F) (8 mg, 28%). Chiral purity 99.2% (Chiralpak IC (4.6×250 mm column), 25% IPA with 0.2% PrNH₂); ¹H NMR (400 MHz, DMSO-d₆): δ ppm 9.01 (s, 1H), 7.99 (s, 1H), 7.69 (br. s, 2H), 6.86-6.83 (m, 2H), 6.73 (d, J=8.4 Hz, 1H), 4.81 (s, 1H), 3.53-3.50 (m, 2H), 3.43-3.40 (m, 1H), 3.30-3.27 (m, 1H), 1.98-1.90 (m, 2H), 1.73 (s, 3H), 1.64 (s, 3H), 1.37 (s, 3H). MS (ESI) m/z: 366.4 [M+H]⁺. Compounds 13-B, 13-C, 13-E, and 13-F are diastereomers, and the shown stereochemistry was arbitrarily assigned.

### Example 14

### 3-((S)-4-amino-6-(((1s,3R)-3-hydroxycyclobutyl)amino)pyrido[3,4-d]pyrimidin-8-yl)-2,4-dimethylphenol (14-B) and 3-((R)-4-amino-6-(((1s,3S)-3-hydroxycyclobutyl)amino)pyrido[3,4-d]pyrimidin-8-yl)-2,4-dimethylphenol (14-C)

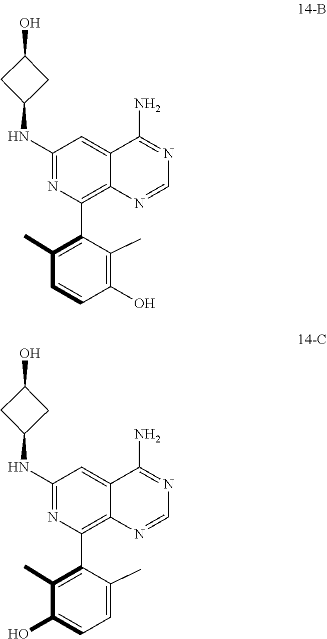

The racemic mixture of 3-(4-amino-6-(((1s,3s)-3-hydroxycyclobutyl)amino)pyrido[3,4-d]pyrimidin-8-yl)-2,4-dimethylphenol (14-A) (0.045 g) was prepared following the procedure described in Example 1 (but replacing DIPEA with K₂CO₃ in NMP in the first step) and was subjected to chiral SFC purification (Chiralpak IC, 55% IPA with 0.2% ⁱPrNH₂) to afford the title compounds. From Peak 1: 3-((S)-4-amino-6-(((1s,3R)-3-hydroxycyclobutyl)amino)pyrido[3,4-d]pyrimidin-8-yl)-2,4-dimethylphenol (14-B) (6.5 mg, 14%). Chiral purity 99.9% (Chiralpak IC (4.6×250 mm column), 30% IPA with 0.2% PrNH₂); ¹H NMR (400 MHz, DMSO-d₆): δ ppm 8.99 (s, 1H), 7.99 (s, 1H), 7.66 (br. s, 2H), 6.88-6.82 (m, 2H), 6.72-6.68 (m, 2H), 5.05 (d, J=6 Hz, 1H), 3.89-3.84 (m, 1H), 3.50-3.44 (m, 1H), 2.77-2.75 (m, 2H), 1.81-1.74 (m, 2H), 1.71 (s, 3H), 1.63 (s, 3H). MS (ESI) m/z: 352.5 [M+H]⁺. From Peak 2: 3-((R)-4-amino-6-(((1s,3S)-3-hydroxycyclobutyl)amino)pyrido[3,4-d]pyrimidin-8-yl)-2,4-dimethylphenol (14-C) (6.5 mg, 14%). Chiral purity 99.7% (Chiralpak IC (4.6×250 mm column), 30% IPA with 0.2% ⁱPrNH₂); ¹H NMR (400 MHz, DMSO-d₆): δ ppm 8.99 (s, 1H), 7.99 (s, 1H), 7.66 (br. s, 2H), 6.88-6.82 (m, 2H), 6.72-6.68 (m, 2H), 5.05 (d, J=6 Hz, 1H), 3.89-3.84 (m, 1H), 3.52-3.44 (m, 1H), 2.79-2.73 (m, 2H), 1.81-1.75 (m, 2H), 1.74 (s, 3H), 1.71 (s, 3H); MS (ESI) m/z: 352.5 [M+H]⁺. Compounds 14-B and 14-C are atropisomers, and the shown stereochemistry may be the opposite of that depicted.

### Example 15

### 3-((S)-4-Amino-6-(((1r,3S)-3-hydroxycyclobutyl)amino)pyrido[3,4-d]pyrimidin-8-yl)-2,4-dimethylphenol (15-B) and 3-((R)-4-amino-6-(((1r,3R)-3-hydroxycyclobutyl)amino) pyrido[3,4-d]pyrimidin-8-yl)-2,4-dimethylphenol (15-C)

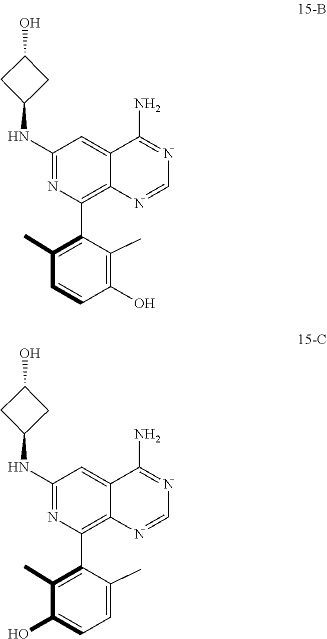

The racemic mixture of 3-(4-amino-6-(((1r,3r)-3-hydroxycyclobutyl)amino)pyrido[3,4-d]pyrimidin-8-yl)-2,4-dimethylphenol (15-A) (0.014 g) was prepared following the procedure described in Example 14 and was subjected to chiral SFC purification (DCPAK P4VP (4.6×250 mm column), 35% MeOH) to afford the title compounds. From Peak 1: 3-((S)-4-amino-6-(((1r,3S)-3-hydroxycyclobutyl)amino)pyrido[3,4-d]pyrimidin-8-yl)-2,4-dimethylphenol (15-B) (35 mg, 21%). Chiral purity 99.9% (Chiralpak IJ (4.6×250 mm column), 15% MeOH with 0.5% DEA); ¹H NMR (400 MHz, DMSO-d₆): δ ppm 8.89 (br. s, 1H), 7.99 (s, 1H), 7.68 (br. s, 2H), 6.93 (d, J=6.4 Hz, 1H), 6.83 (d, J=8.0 Hz, 1H), 6.71 (d, J=8.0 Hz, 1H), 6.63 (s, 1H), 5.02 (br. s, 1H), 4.47-4.28 (m, 1H), 4.09-4.00 (m, 1H), 2.30-2.16 (m, 4H), 1.71 (s, 3H), 1.63 (s, 3H). MS (ESI) m/z: 352.3 [M+H]⁺. From Peak 2: 3-((R)-4-amino-6-(((1r,3R)-3-hydroxycyclobutyl)amino)pyrido[3,4-d]pyrimidin-8-yl)-2,4-dimethylphenol (15-C) (37.4 mg, 21%). Chiral purity 98.6% (Chiralpak IJ (4.6×250 mm column), 15% MeOH with 0.5% DEA); ¹H NMR (400 MHz, DMSO-d₆): δ ppm 9.00 (s, 1H), 7.99 (s, 1H), 7.69 (br. s, 2H), 6.93 (d, J=6.4 Hz, 1H), 6.83 (d, J=8.0 Hz, 1H), 6.71 (d, J=8.4 Hz, 1H), 6.63 (s, 1H), 5.01 (d, J=5.2 Hz, 1H), 4.37-4.28 (m, 1H), 4.10-4.00 (m, 1H), 2.30-2.16 (m, 4H), 1.71 (s, 3H), 1.63 (s, 3H). MS (ESI) m/z: 352.3 [M+H]⁺. Compounds 15-B and 15-C are atropisomers, and the shown stereochemistry may be the opposite of that depicted.

### Example 16

### (S)—N-(4-amino-8-(3-hydroxy-2,6-dimethylphenyl)pyrido[3,4-d]pyrimidin-6-yl)methanesulfonamide (16-B) and (R)—N-(4-amino-8-(3-hydroxy-2,6-dimethylphenyl)pyrido[3,4-d]pyrimidin-6-yl)methanesulfonamide (16-C)

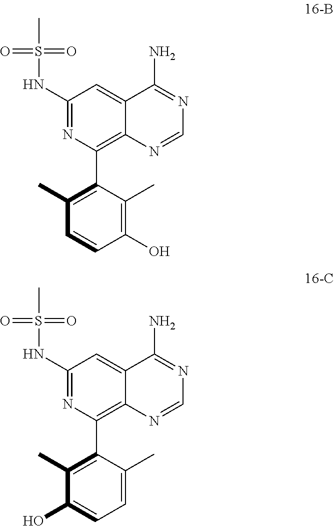

The racemic mixture N-(4-amino-8-(3-hydroxy-2,6-dimethylphenyl)pyrido[3,4-d]pyrimidin-6-yl)methanesulfonamide (0.07 g) was prepared according to Example 8 and was subjected to chiral SFC purification (ChiralCel-OD-3, 30×250 mm column, 25% MeOH with 10 mM NH₄HCO₃) to afford the title compounds. From Peak 1: (S)—N-(4-amino-8-(3-hydroxy-2,6-dimethylphenyl)pyrido[3,4-d]pyrimidin-6-yl)methanesulfonamide (16-B) (7.17 mg, 10%). Chiral purity 99.8%, (Chiralcel OD-3 (4.6×150 mm column), 25% MeOH); ¹H-NMR (400 MHz, DMSO-d₆): δ ppm 10.64 (s, 1H), 9.11 (s, 1H), 8.26 (s, 1H), 8.10 (br. s, 2H), 7.65 (s, 1H), 6.88 (d, J=8 Hz, 1H), 6.76 (d, J=8.4 Hz, 1H), 3.23 (s, 3H), 1.72 (s, 3H), 1.63 (s, 3H). MS (ESI) m/z: 360.4 [M+H]⁺. From Peak 2: (R)—N-(4-amino-8-(3-hydroxy-2,6-dimethylphenyl)pyrido[3,4-d]pyrimidin-6-yl)methanesulfonamide (16-C) (5.05 mg, 7%). Chiral purity 95.7% (Chiralcel OD-3 (4.6×150 mm column), 25% MeOH); ¹H-NMR (400 MHz, DMSO-d₆): δ ppm 8.90 (s, 1H), 7.91 (s, 1H), 7.46 (br. s, 2H), 6.86 (br. s, 1H), 6.81 (d, J=8 Hz, 1H), 6.69 (d, J=8.4 Hz, 1H), 2.77 (s, 3H), 1.73 (s, 3H), 1.65 (s, 3H). MS (ESI) m/z: 360.4 [M+H]⁺. Compounds 16-B and 16-C are atropisomers, and the shown stereochemistry may be the opposite of that depicted.

### Example 17

### (S)-3-(4-Amino-6-methylpyrido[3,4-d]pyrimidin-8-yl)-2,4-dimethylphenol (17-B) and (R)-3-(4-amino-6-methylpyrido[3,4-d]pyrimidin-8-yl)-2,4-dimethylphenol (17-C)

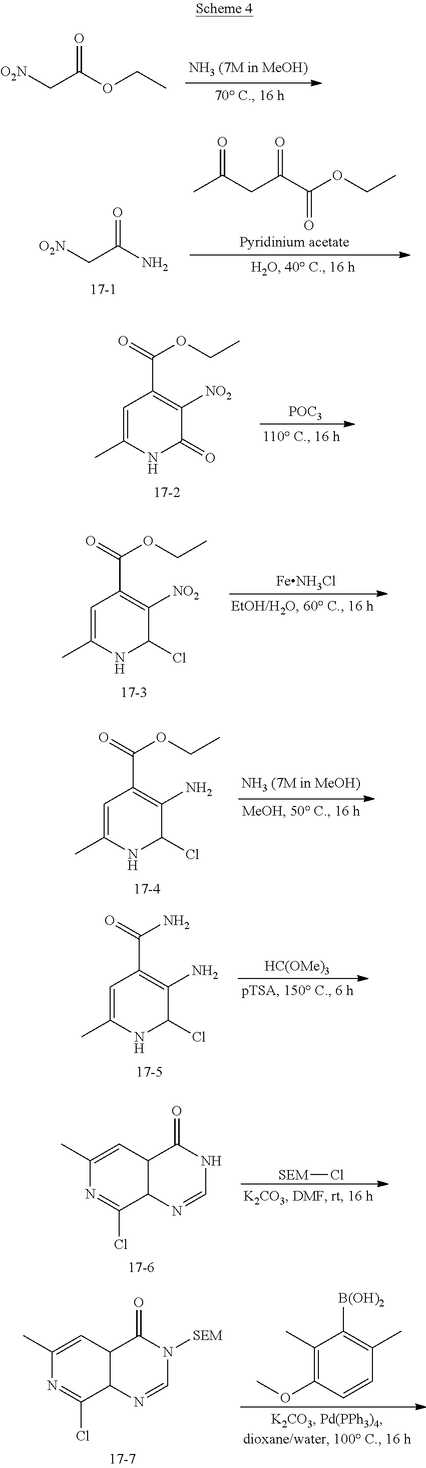

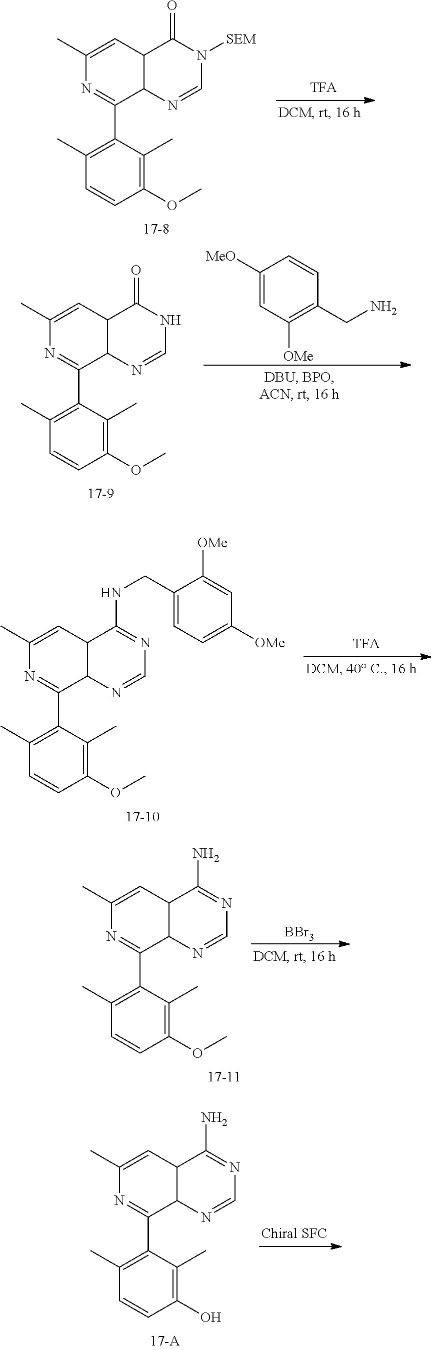

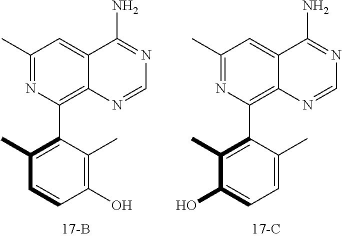

### 2-nitroacetamide (17-1)

A solution of ethyl 2-nitroacetate (25 g, 187.82 mmol) in NH₃ solution (7M in MeOH, 401 mL) was stirred at 70° C. for 16 h. The reaction was performed in autoclave. After completion, the mixture was concentrated under reduced pressure to afford 2-nitroacetamide (17-1) (22.00 g, quantitative). The material was taken to the next step without further purification. MS (ESI): m/z 105.11 [M+H]⁺.

### ethyl 6-methyl-3-nitro-2-oxo-1,2-dihydropyridine-4-carboxylate (17-2)

To a solution of 2-nitroacetamide (17-1) (22.00 g, 211.41 mmol) in water (440 mL) were added ethyl 2,4-dioxopentanoate (40.12 g, 253.69 mmol) followed by a solution of pyridinium acetate (92.09 mL, 634.22 mmol) prepared by adding pyridine (13 mL) to 6.5 mL of AcOH (6.5 mL) in water (26 mL). The mixture was stirred at 40° C. for 16 h. After completion, the mixture was cooled in an ice bath. The precipitated product was filtered and washed with cold water (50 mL) to afford ethyl 6-methyl-3-nitro-2-oxo-1,2-dihydropyridine-4-carboxylate (17-2) (11 g, 23%). The material was taken to the next step without further purification. MS (ESI): m/z 227.28 [M+H]⁺.

### ethyl 2-chloro-6-methyl-3-nitroisonicotinate (17-3)

A solution of ethyl 6-methyl-3-nitro-2-oxo-1,2-dihydropyridine-4-carboxylate (17-2) (11.00 g, 48.63 mmol) in P(O)Cl₃ (110 mL) was stirred at 110° C. for 16 h. The reaction mixture was concentrated under reduced pressure. The residue was diluted with water and basified with sat. NaHCO₃ solution and then extracted with EtOAc (3×300 mL). The combined organic layers were washed with brine (100 mL), dried over MgSO₄, filtered, and concentrated. The residue was purified by column chromatography (SiO₂, 9% EtOAc/PET) to afford ethyl 2-chloro-6-methyl-3-nitroisonicotinate (17-3) (9.20 g, 77%). ¹H-NMR (400 MHz, DMSO-d₆) δ 7.94 (s, 1H), 4.36 (q, J=7.1 Hz, 2H), 2.64 (s, 3H), 1.29 (t, J=7.0 Hz, 3H). MS (ESI): m/z 244.92 [M+H]⁺.

### ethyl 3-amino-2-chloro-6-methylisonicotinate (17-4)

To a solution of ethyl 2-chloro-6-methyl-3-nitroisonicotinate (17-3) (9.20 g, 37.61 mmol) in EtOH (92 mL) and water (27.6 mL) was added iron powder (14.70 g, 263.25 mmol) and NH₄Cl (14.08 g, 263.25 mmol). The solution was stirred at 60° C. for 16 h. The mixture was filtered over Celite and washed with EtOAc (100 mL). The filtrate was concentrated under reduced pressure. The residue was purified by column chromatography (SiO₂, Devisil silica, 8% EtOAc/PET) to afford ethyl 3-amino-2-chloro-6-methylisonicotinate (17-4) (5.40 g, 66%); ¹H-NMR (400 MHz, DMSO-d₆) δ ppm 7.47 (s, 1H), 6.53 (br, s, 2H), 4.32 (q, J=7.1 Hz, 2H), 2.32 (s, 3H), 1.33 (t, J=7.2 Hz, 3H). MS (ESI): m/z 214.73[M+H]⁺.

### 3-amino-2-chloro-6-methylisonicotinamide (17-5)

Ethyl 3-amino-2-chloro-6-methylisonicotinate (17-4) (5.40 g, 25.16 mmol) was dissolved in NH₃ (7N in MeOH, 68 mL), and the mixture was stirred at 50° C. for 6 h. After completion, the mixture was concentrated under reduced pressure to afford 3-amino-2-chloro-6-methylisonicotinamide (17-5) (4.50 g, 97%). MS (ESI): m/z 186.09 [M+H]⁺. This material was taken to the next step without further purification.

### 8-chloro-6-methylpyrido[3,4-d]pyrimidin-4 (3H)-one (17-6)

To a solution of 3-amino-2-chloro-6-methylisonicotinamide (17-5) (4.50 g, 24.24 mmol) in trimethyl orthoformate (135 mL) was added p-TSA (0.21 g, 1.21 mmol) in autoclave at rt. The mixture was stirred at 150° C. for 8 h. After completion, the mixture was concentrated under reduced pressure to afford 8-chloro-6-methylpyrido[3,4-d]pyrimidin-4 (3H)-one (17-6) (6.0 g, quantitative). MS (ESI): m/z 196.31 [M+3H]⁺. This material was taken to the next step without further purification.

### 8-chloro-6-methyl-3-((2-(trimethylsilyl)ethoxy)methyl)pyrido[3,4-d]pyrimidin-4 (3H)-one (17-7)

To a solution of 8-chloro-6-methylpyrido[3,4-d]pyrimidin-4 (3H)-one (17-6) (6.00 g, 30.67 mmol) in DMF (60 mL) was added anhydrous K₂CO₃ (12.72 g, 92.02 mmol). The mixture was stirred at 60° C. for 15 min., and then SEM-Cl (16.29 mL, 92.02 mmol) was added dropwise. The mixture was stirred at same temperature for 16 h. After completion, the mixture was poured into ice water, precipitation was filtered and washed with water (50 mL), and then dried under vacuum to afford 8-chloro-6-methyl-3-((2-(trimethylsilyl)ethoxy)methyl)pyrido-[3,4-d]pyrimidin-4 (3H)-one (17-7) (10.00 g, 100%). ¹H-NMR (400 MHz, DMSO-d₆) δ ppm 8.57 (s, 1H), 7.90 (s, 1H), 5.37 (s, 2H), 3.61 (t, J=8.0 Hz, 2H), 2.59 (s, 3H), 0.86 (t, J=4.0 Hz, 2H), −0.04 (s, 9H). MS (ESI): m/z 326.39 [M+H]⁺.

### 8-(3-methoxy-2,6-dimethylphenyl)-6-methyl-3-((2-(trimethylsilyl)ethoxy)methyl)pyrido[3,4-d]pyrimidin-4 (3H)-one (17-8)

To a solution of 8-chloro-6-methyl-3-((2-(trimethylsilyl)ethoxy)methyl)pyrido[3,4-d]pyrimidin-4 (3H)-one (17-7) (0.80 g, 2.45 mmol) in a mixture of 1,4-dioxane and water (20 mL, 8:2) were added (3-methoxy-2,6-dimethylphenyl)boronic acid (0.80 g, 4.42 mmol) and K₂CO₃ (1.36 g, 9.82 mmol). The mixture was degassed with N₂ for 5 min. followed by addition of Pd(PPh₃)₄ (0.28 g, 0.25 mmol). The mixture was degassed again with N₂ for 3 min. and stirred at 100° C. for 16 h. After completion, the reaction was quenched with water (50 mL) and extracted with EtOAc (2×50 mL). The combined organic layers were washed with brine (50 mL), dried over MgSO₄, filtered, and concentrated under reduced pressure. The residue was purified by column chromatography (SiO₂, 20% EtOAc/PET) to afford 8-(3-methoxy-2,6-dimethylphenyl)-6-methyl-3-((2-(trimethylsilyl)ethoxy)methyl)pyrido[3,4-d]pyrimidin-4 (3H)-one (17-8) (0.80 g, 76%). MS (ESI): m/z 426.5 [M+H]⁺.

### 8-(3-methoxy-2,6-dimethylphenyl)-6-methylpyrido[3,4-d]pyrimidin-4 (3H)-one (17-9)

To a solution of 8-(3-methoxy-2,6-dimethylphenyl)-6-methyl-3-((2-(trimethylsilyl)ethoxy)methyl)pyrido[3,4-d]pyrimidin-4 (3H)-one (17-8) (0.80 g, 1.88 mmol) in CH₂Cl₂ (16 mL) was added TFA (1.53 mL, 18.80 mmol). The mixture was stirred at rt for 16 h. After completion, the reaction was carefully quenched with sat. NaHCO₃ (30 mL) and extracted with EtOAc (3×50 mL). The combined organic layers were washed with brine (50 mL), dried over Na₂SO₄, and filtered, and concentrated. The residue was triturated in pentane (2×5 mL) to afford 8-(3-methoxy-2,6-dimethylphenyl)-6-methylpyrido[3,4-d]pyrimidin-4 (3H)-one (17-9) (0.45 g, 81%). MS (ESI): m/z 296.3 [M+H]⁺.

### N-(2,4-dimethoxybenzyl)-8-(3-methoxy-2,6-dimethylphenyl)-6-methylpyrido[3,4-d]pyrimidin-4-amine (17-10)

To a solution of 8-(3-methoxy-2,6-dimethylphenyl)-6-methylpyrido[3,4-d]pyrimidin-4 (3H)-one (17-9) (0.45 g, 1.52 mmol) and (2,4-dimethoxyphenyl)methanamine (0.51 g, 3.05 mmol) in ACN (9 mL) were added BOP (0.88 g, 1.98 mmol) and DBU (0.35 g, 2.29 mmol) at rt. The mixture was stirred at rt for 16 h. After completion, the mixture was poured into an ice-cold water (30 mL). The resulting product was filtered and then triturated with pentane (2×5 mL) to afford N-(2,4-dimethoxybenzyl)-8-(3-methoxy-2,6-dimethylphenyl)-6-methylpyrido[3,4-d]pyrimidin-4-amine (17-10) (0.60 g, 88%). MS (ESI): m/z 445.53 [M+H]⁺.

### 8-(3-methoxy-2,6-dimethylphenyl)-6-methylpyrido[3,4-d]pyrimidin-4-amine (17-11)

To a solution of N-(2,4-dimethoxybenzyl)-8-(3-methoxy-2,6-dimethylphenyl)-6-methylpyrido[3,4-d]pyrimidin-4-amine (17-10) (0.60 g, 1.35 mmol) in CH₂Cl₂ (12 mL) was added TFA (2.07 mL, 26.99 mmol) at rt. The mixture was stirred at 40° C. for 16 h. After completion, the reaction was carefully quenched with aqueous NaHCO₃ (50 mL) and extracted with EtOAc (3×50 mL). The combined organic layers were washed with brine (50 mL), dried over Na₂SO₄, filtered, and concentrated. The residue was purified by column chromatography (SiO₂, 80% EtOAc/PET) to afford 8-(3-methoxy-2,6-dimethylphenyl)-6-methylpyrido[3,4-d]pyrimidin-4-amine (17-11) (0.26 g, 65%). MS (ESI): m/z 295.2 [M+H]⁺.

### 3-(4-amino-6-methylpyrido[3,4-d]pyrimidin-8-yl)-2,4-dimethylphenol (17-A)

To a solution of 8-(3-methoxy-2,6-dimethylphenyl)-6-methylpyrido[3,4-d]pyrimidin-4-amine (17-11) (0.25 g, 0.85 mmol) in DCM (5 mL) was added BBr₃ (1M in CH₂Cl₂, 4.2 mL, 4.25 mmol). The mixture was stirred at rt for 16 h. After completion, the reaction mixture was concentrated to dryness, cooled in an ice bath, and diluted with sat. NaHCO₃ (50 mL). The mixture was extracted with EtOAc (3×50 mL). The combined organic layers were washed with brine (30 mL), dried over Na₂SO₄, filtered, and concentrated. The residue was purified by prep-HPLC (C18, ACN/H₂O with 10 mM NH₄HCO₃) to get 3-(4-amino-6-methylpyrido[3,4-d]pyrimidin-8-yl)-2,4-dimethylphenol (17-A) (55 mg, 23%). MS (ESI): m/z 281.3 [M+H]⁺.

### Isolation of (S)-3-(4-amino-6-methylpyrido[3,4-d]pyrimidin-8-yl)-2,4-dimethylphenol (17-B) and (R)-3-(4-amino-6-methylpyrido[3,4-d]pyrimidin-8-yl)-2,4-dimethylphenol (17-C)

The racemic 3-(4-amino-6-methylpyrido[3,4-d]pyrimidin-8-yl)-2,4-dimethylphenol (17-A) (0.053 g) was subjected to chiral SFC purification to afford the title compounds. From Peak 1: (S)-3-(4-amino-6-methylpyrido[3,4-d]pyrimidin-8-yl)-2,4-dimethylphenol (17-B): (22 mg, 41%). Chiral purity 99.9% (Chiralpak AD-H (4.6×250 mm column), 15% MeOH with 0.5% DEA); ¹H NMR (400 MHz, DMSO-d₆): δ ppm 9.05 (s, 1H), 8.30 (s, 1H), 8.01-7.95 (m, 3H), 6.87 (d, J=8.0 Hz, 1H), 6.75 (d, J=8.0 Hz, 1H), 2.61 (s, 3H), 1.67 (s, 3H), 1.59 (s, 3H). MS (ESI): m/z 281.1 [M+H]⁺. From Peak 2: (R)-3-(4-amino-6-methylpyrido[3,4-d]pyrimidin-8-yl)-2,4-dimethylphenol (17-C): (23 mg, 43%). Chiral purity 98.3% (Chiralpak AD-H (4.6×250 mm column), 15% MeOH with 0.5% DEA. ¹H NMR (400 MHz, DMSO-d₆): δ ppm 9.05 (s, 1H), 8.30 (s, 1H), 8.01-7.95 (m, 3H), 6.87 (d, J=8.0 Hz, 1H), 6.75 (d, J=8.0 Hz, 1H), 2.61 (s, 3H), 1.67 (s, 3H), 1.59 (s, 3H). MS (ESI): m/z 281.11 [M+H]⁺. Compounds 17-B and 17-C are atropisomers, and the shown stereochemistry may be the opposite of that depicted.

### Example 18

### (S)-3-(4-Amino-6-cyclopropylpyrido[3,4-d]pyrimidin-8-yl)-2,4-dimethylphenol (18-B) and (R)-3-(4-amino-6-cyclopropylpyrido[3,4-d]pyrimidin-8-yl)-2,4-dimethylphenol (18-C)

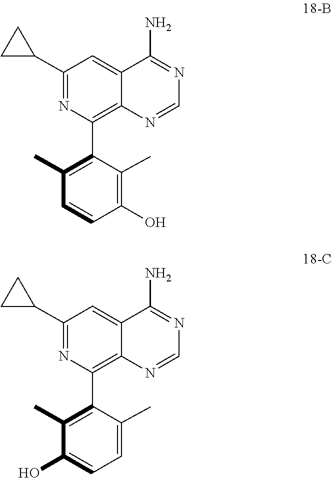

The racemic mixture of 3-(4-amino-6-cyclopropylpyrido[3,4-d]pyrimidin-8-yl)-2,4-dimethylphenol was prepared according to Example 1, with the following variation regarding the first step: to a solution of 6-chloro-8-(3-methoxy-2,6-dimethylphenyl)-3-((2-(trimethylsilyl)ethoxy)methyl)pyrido[3,4-d]pyrimidin-4 (3H)-one (0.80 g, 1.79 mmol) and cyclopropylboronic acid (0.46 g, 5.38 mmol) in 1,4-dioxane:water (3:1, 4 mL) was added K₂CO₃ (0.99 g, 7.18 mmol). The mixture was degassed with N₂ for 5 min. followed by addition of Pd(dppf)Cl₂·CH₂Cl₂ (0.15 g, 0.18 mmol). The mixture was degassed again for 5 min., then heated at 100° C. for 16 h.

The racemic mixture of 3-(4-amino-6-cyclopropylpyrido[3,4-d]pyrimidin-8-yl)-2,4-dimethylphenol (18-A) (0.07 g) was subjected to chiral SFC (LUX-i-Amylose-3, 30×250 mm column, 20% MeOH) to afford the title compounds. From Peak 1: (S)-3-(4-amino-6-cyclopropylpyrido[3,4-d]pyrimidin-8-yl)-2,4-dimethylphenol (18-B) (35 mg, 50%). Chiral purity 99.97% (Lux i-Amylose-3 (4.6×250 mm column), 20% MeOH); ¹H NMR (400 MHz, DMSO-d₆): δ ppm 9.06 (s, 1H), 8.26 (s, 1H), 7.99 (br. s, 2H), 7.94 (s, 1H), 6.86 (d, J=8.4 Hz, 1H), 6.74 (d, J=8.4 Hz, 1H), 2.20-2.14 (m, 1H), 1.66 (s, 3H), 1.57 (s, 3H), 1.03-0.99 (m, 2H), 0.93-0.87 (m, 2H). MS (ESI) 307.1 [M+H]⁺. From Peak 2: (R)-3-(4-amino-6-cyclopropylpyrido[3,4-d]pyrimidin-8-yl)-2,4-dimethylphenol (18-C) (24 mg, 34% yield). Chiral purity 99.43% (Lux i-Amylose-3 (4.6×250 mm column), 20% MeOH); ¹H NMR (400 MHz, DMSO-d₆): δ ppm 9.06 (s, 1H), 8.26 (s, 1H), 7.99 (br. s, 2H), 7.94 (s, 1H), 6.86 (d, J=8.0 Hz, 1H), 6.74 (d, J=8.0 Hz, 1H), 2.20-2.14 (m, 1H), 1.66 (s, 3H), 1.57 (s, 3H), 1.04-0.97 (m, 2H), 0.92-0.87 (m, 2H); MS (ESI) 307.4 [M+H]⁺. Compounds 18-B and 18-C are atropisomers, and the shown stereochemistry may be the opposite of that depicted.

### Example 19

### (S)-8-(3-Methoxy-2,6-dimethylphenyl)-6-(1-methyl-1H-pyrazol-4-yl)pyrido[3,4-d]pyrimidin-4-amine (19-B) and (R)-8-(3-methoxy-2,6-dimethylphenyl)-6-(1-methyl-1H-pyrazol-4-yl)pyrido[3,4-d]pyrimidin-4-amine (19-C)

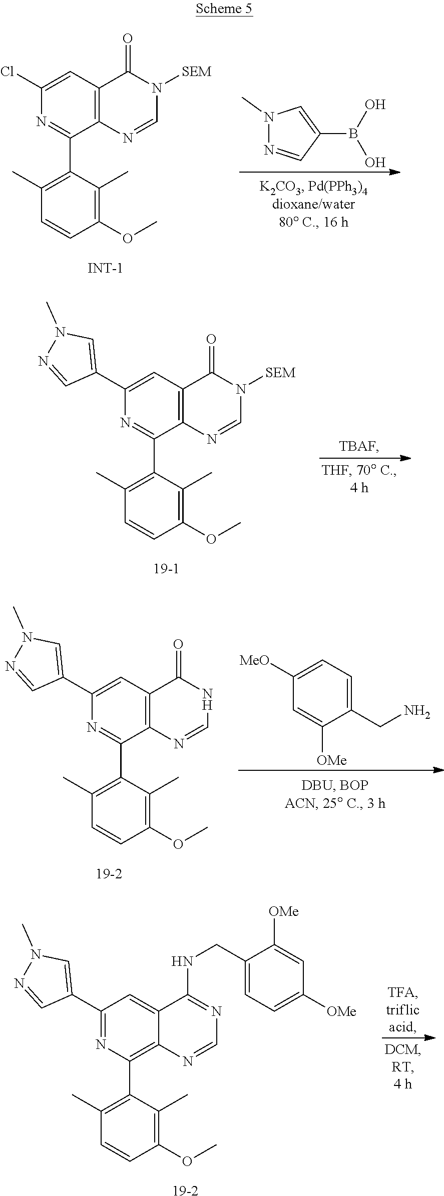

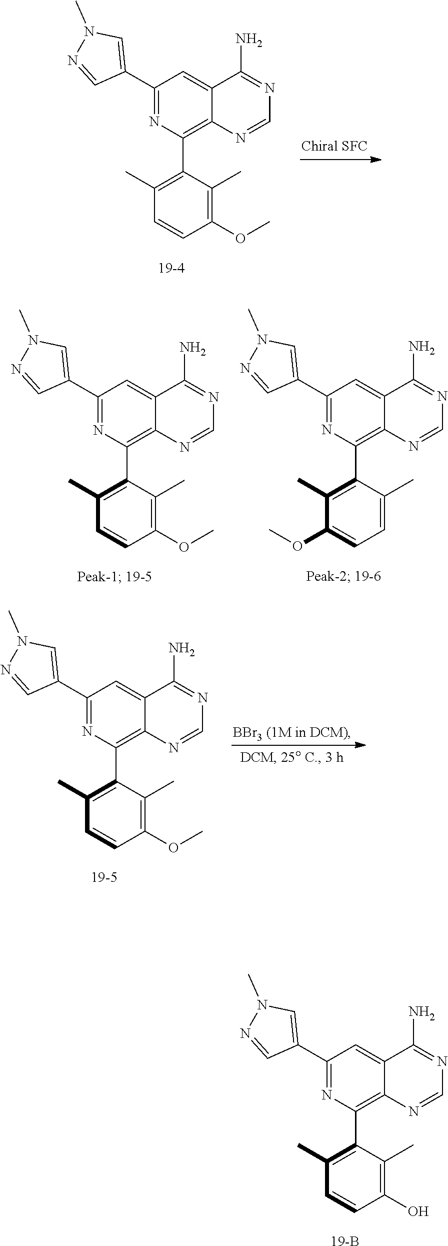

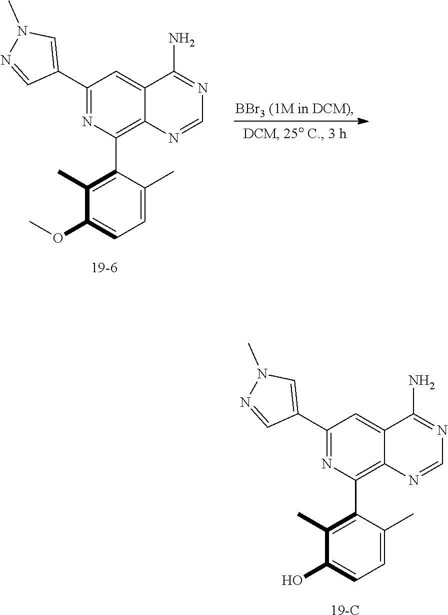

### 8-(3-methoxy-2,6-dimethylphenyl)-6-(1-methyl-1H-pyrazol-4-yl)-3-((2-(trimethylsilyl)ethoxy)methyl)pyrido[3,4-d]pyrimidin-4 (3H)-one (19-1)

To a solution of 6-chloro-8-(3-methoxy-2,6-dimethylphenyl)-3-((2-(trimethylsilyl)ethoxy)methyl)pyrido[3,4-d]pyrimidin-4 (3H)-one (INT-1) (1.00 g, 2.24 mmol) and (1-methyl-1H-pyrazol-4-yl)boronic acid (0.51 mg, 4.04 mmol) in 1,4-dioxane:water (4.7:1, 40 mL) was added K₂CO₃ (0.93 g, 6.73 mmol) and degassed mixture with N₂ for 5 min. followed by addition of Pd(PPh₃)₄ (0.60 g, 0.22 mmol). The mixture was degassed again for 3 min. and heated at 80° C. for 16 h. After completion, the mixture was diluted with water (30 mL) and extracted with EtOAc (2×30 mL). The organic layer was dried over MgSO₄, filtered, and concentrated. The residue was purified by column chromatography (SiO₂, EtOAc/PET) to afford 8-(3-methoxy-2,6-dimethylphenyl)-6-(1-methyl-1H-pyrazol-4-yl)-3-((2-(trimethylsilyl)ethoxy)methyl)pyrido[3,4-d]pyrimidin-4 (3H)-one (19-1) (1.06 g, 96%). MS (ESI) 492.4 [M+H]⁺.

### 8-(3-methoxy-2,6-dimethylphenyl)-6-(1-methyl-1H-pyrazol-4-yl)pyrido[3,4-d]pyrimidin-4 (3H)-one (19-2)

To a solution of 8-(3-methoxy-2,6-dimethylphenyl)-6-(1-methyl-1H-pyrazol-4-yl)-3-((2-(trimethylsilyl)ethoxy)methyl)pyrido[3,4-d]pyrimidin-4 (3H)-one (19-1) (1.00 g, 2.03 mmol) in THF (20 mL) was added TBAF (1M in THF, 8.14 mL, 8.14 mmol) at rt, then the mixture was heated at 80° C. for 3 h. After completion, the mixture was diluted with water (30 mL) and extracted with EtOAc (2×30 mL). The organic layers were dried over MgSO₄, filtered, and concentrated to afford 8-(3-methoxy-2,6-dimethylphenyl)-6-(1-methyl-1H-pyrazol-4-yl)pyrido[3,4-d]pyrimidin-4 (3H)-one (19-2) (0.91 g, quantitative). MS (ESI) 362.2 [M+H]⁺. The material was taken to the next step without further purification.

### N-(2,4-dimethoxybenzyl)-8-(3-methoxy-2,6-dimethylphenyl)-6-(1-methyl-1H-pyrazol-4-yl)pyrido[3,4-d]pyrimidin-4-amine (19-3)

To a solution of 8-(3-methoxy-2,6-dimethylphenyl)-6-(1-methyl-1H-pyrazol-4-yl)pyrido[3,4-d]pyrimidin-4 (3H)-one (19-2) (0.90 g, 2.49 mmol) and (2,4-dimethoxyphenyl)methanamine (0.75 mL, 4.98 mmol) in ACN (18 mL) were added BOP (1.43 g, 3.24 mmol) and DBU (0.56 mL, 3.74 mmol) at rt. The mixture was stirred at rt for 16 h. After completion, the reaction was quenched with H₂O (30 mL) and extracted with EtOAc (3×30 mL). The combined organic layers were washed with brine (30 mL), dried over Na₂SO₄, filtered, and concentrated. The residue was purified by column chromatography (SiO₂, EtOAc/PET) to afford N-(2,4-dimethoxybenzyl)-8-(3-methoxy-2,6-dimethylphenyl)-6-(1-methyl-1H-pyrazol-4-yl)pyrido[3,4-d]pyrimidin-4-amine (19-3) (1.4 g, quantitative). MS (ESI) 511.7 [M+H]⁺. The material was taken to the next step without further purification.

### 8-(3-methoxy-2,6-dimethylphenyl)-6-(1-methyl-1H-pyrazol-4-yl)pyrido[3,4-d]pyrimidin-4-amine (19-4)

To a solution of N-(2,4-dimethoxybenzyl)-8-(3-methoxy-2,6-dimethylphenyl)-6-(1-methyl-1H-pyrazol-4-yl)pyrido[3,4-d]pyrimidin-4-amine (19-3) (1.40 g, 2.74 mmol) in CH₂Cl₂ (14 mL) were added TFA (2.10 mL, 27.42 mmol) followed by TfOH (3.63 mL, 41.13 mmol) at rt. The mixture was stirred at rt for 1.5 h. After completion, the mixture was diluted with ice water (20 mL), basified with sat. NaHCO₃ and extracted CH₂C₁₂ (3×20 mL). The organic layers were separated, dried over MgSO₄, and filtered, then concentrated under reduced pressure. The residue was purified by column chromatography (SiO₂, EtOAc/PET) to afford 8-(3-methoxy-2,6-dimethylphenyl)-6-(1-methyl-1H-pyrazol-4-yl)pyrido[3,4-d]pyrimidin-4-amine (19-4) (0.40 g, 40%). MS (ESI) 361.5 [M+H]⁺.

### (S)-8-(3-methoxy-2,6-dimethylphenyl)-6-(1-methyl-1H-pyrazol-4-yl)pyrido[3,4-d]pyrimidin-4-amine (19-5) and (R)-8-(3-methoxy-2,6-dimethylphenyl)-6-(1-methyl-1H-pyrazol-4-yl)pyrido[3,4-d]pyrimidin-4-amine (19-6)

The racemic mixture of 8-(3-methoxy-2,6-dimethylphenyl)-6-(1-methyl-1H-pyrazol-4-yl)pyrido[3,4-d]pyrimidin-4-amine (19-4) (0.40 g) was subjected to chiral SFC (Chiralpak ADH, 30% MeOH) to afford the title compounds. From Peak 1: (S)-8-(3-methoxy-2,6-dimethylphenyl)-6-(1-methyl-1H-pyrazol-4-yl)pyrido[3,4-d]pyrimidin-4-amine (19-5) (11 mg, 27%). MS (ESI) 361.2 [M+H]⁺. From Peak 2: (R)-8-(3-methoxy-2,6-dimethylphenyl)-6-(1-methyl-1H-pyrazol-4-yl)pyrido[3,4-d]pyrimidin-4-amine (19-6) (11 mg, 26%). MS (ESI) 361.1 [M+H]⁺. Compounds 19-5 and 19-6 are atropisomers, and the shown stereochemistry may be the opposite of that depicted.

### (S)-3-(4-amino-6-(1-methyl-1H-pyrazol-4-yl)pyrido[3,4-d]pyrimidin-8-yl)-2,4-dimethylphenol (19-7)

To a stirred solution of (S)-8-(3-methoxy-2,6-dimethylphenyl)-6-(1-methyl-1H-pyrazol-4-yl)pyrido[3,4-d]pyrimidin-4-amine (19-5) (0.10 g, 0.28 mmol) in CH₂Cl₂ (1.00 mL) was added BBr₃ (1M in CH₂Cl₂, 1.39 mL, 1.39 mmol) at 0° C. and stirred the reaction mixture at rt for 2 h. After completion, the mixture was diluted with CH₂Cl₂ (10 mL) and basified with sat. NaHCO₃ to pH~8. The organic layer was washed with H₂O (2×10 mL), separated, dried over MgSO₄, filtered, and concentrated. The residue was purified by preparative HPLC (X-bridge C18, 19×250 mm column, Water/ACN/0.2% FA) to afford (S)-3-(4-amino-6-(1-methyl-1H-pyrazol-4-yl)pyrido[3,4-d]pyrimidin-8-yl)-2,4-dimethylphenol (19-7) (9.2 mg, 9%). Chiral purity 99.5% (Lux i-Amylose-3 (4.6×250 mm column), 40% iPrOH with 0.5% ⁱPrNH₂); ¹H NMR (400 MHz, DMSO-d₆): δ ppm 9.08 (s, 1H), 8.38 (s, 1H), 8.29 (s, 1H), 8.21 (s, 1H), 8.05 (br. s, 2H), 7.97 (s, 1H), 6.88 (d, J=8.4 Hz, 1H), 6.77 (d, J=8.4 Hz, 1H), 3.88 (s, 3H), 1.72 (s, 3H), 1.64 (s, 3H). MS (ESI) 347.4 [M+H]⁺.

### (R)-3-(4-amino-6-(1-methyl-1H-pyrazol-4-yl)pyrido[3,4-d]pyrimidin-8-yl)-2,4-dimethylphenol (19-8)

To a solution of (R)-8-(3-methoxy-2,6-dimethylphenyl)-6-(1-methyl-1H-pyrazol-4-yl)pyrido[3,4-d]pyrimidin-4-amine (19-6) (0.10 g, 0.28 mmol) in CH₂Cl₂ (1 mL) was added BBr₃ (1M in CH₂Cl₂, 1.39 mL, 1.39 mmol) at 0° C. then stirred at rt for 2 h. After completion, the mixture was diluted with CH₂Cl₂ (10 mL), and then basified with sat. NaHCO₃ to pH~8. The organic layer was washed with H₂O (2×10 mL), separated, dried over MgSO₄, filtered, and concentrated. The residue was purified by prep-HPLC (X-bridge C18, 19×250 mm column, H₂O/ACN/0.20% FA) to afford (R)-3-(4-amino-6-(1-methyl-1H-pyrazol-4-yl)pyrido[3,4-d]pyrimidin-8-yl)-2,4-dimethylphenol (19-8) (16.64 mg, 17%). Chiral purity 99.4% (Lux i-Amylose-3, 4.6×250 mm column, 40% iPrOH with 0.5% ⁱPrNH₂); ¹H NMR (400 MHz, DMSO-d₆): δ ppm 9.08 (s, 1H), 8.38 (s, 1H), 8.29 (s, 1H), 8.21 (s, 1H), 8.05 (s, 2H), 7.97 (s, 1H), 6.88 (d, J=8.4 Hz, 1H), 6.77 (d, J=8.4 Hz, 1H), 3.88 (s, 3H), 1.72 (s, 3H), 1.64 (s, 3H). MS (ESI) 347.4 [M+H]⁺.

### Example 20

### (S)-3-(4-Amino-6-(3,3-difluoropiperidin-1-yl)pyrido[3,4-d]pyrimidin-8-yl)-2,4-dimethylphenol (20-B) and (R)-3-(4-amino-6-(3,3-difluoropiperidin-1-yl)pyrido[3,4-d]pyrimidin-8-yl)-2,4-dimethylphenol (20-C)

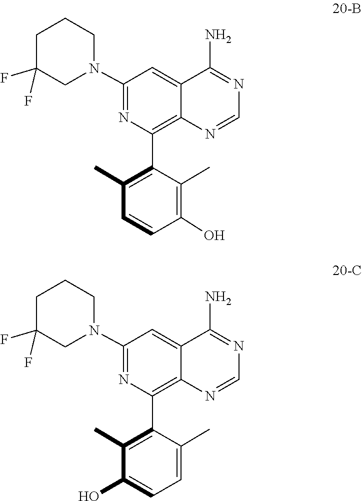

The racemic mixture of 3-(4-amino-6-(3,3-difluoropiperidin-1-yl)pyrido[3,4-d]pyrimidin-8-yl)-2,4-dimethylphenol (20-A) (0.16 g) was prepared following the procedure described in Example 2 (replacing Xphos with Xantphos in 1,4-dioxane in the first step) was subjected to chiral SFC purification (Chiralcel OJ-H, 30×250 mm column, 15% ACN:MeOH (1:1) with 0.2% NH₃ in MeOH) to afford the title compounds. From Peak 1: (S)-3-(4-amino-6-(3,3-difluoropiperidin-1-yl)pyrido[3,4-d]pyrimidin-8-yl)-2,4-dimethylphenol (20-B) (69.4 mg, 27%). Chiral purity 99.3% (Chiralcel OJ-H (4.6×250 mm column), 15% ACN/H2O (1:1) with 0.2% 7N NH₃ in MeOH); ¹H NMR (400 MHz, DMSO-d₆): δ ppm 9.06 (s, 1H), 8.09 (s, 1H), 7.83 (br. s, 2H), 7.37 (s, 1H), 6.86 (d, J=8.0 Hz, 1H), 6.74 (d, J=8.0 Hz, 1H), 3.98-3.92 (m, 2H), 3.69-3.60 (m, 2H), 2.17-2.03 (m, 2H), 1.83-1.73 (m, 2H), 1.71 (s, 3H), 1.63 (s, 3H); MS (ESI) m/z: 386.4 [M+H]⁺. From Peak 2: (R)-3-(4-amino-6-(3,3-difluoropiperidin-1-yl)pyrido[3,4-d]pyrimidin-8-yl)-2,4-dimethylphenol (20-C): (67 mg, 26%). Chiral purity 98.2% (Chiralcel OJ-H (4.6×250 nm column (15% ACN/H₂O (1:1) with 0.2% 7N NH₃ in MeOH); ¹H NMR (400 MHz, DMSO-d₆): δ ppm 9.07 (s, 1H), 8.09 (s, 1H), 7.84 (br. s, 2H), 7.38 (s, 1H), 6.87 (d, J=8.0 Hz, 1H), 6.75 (d, J=8.0 Hz, 1H), 4.00-3.90 (m, 2H), 3.70-3.60 (m, 2H), 2.18-2.06 (m, 2H), 1.82-1.73 (m, 2H), 1.72 (s, 3H), 1.63 (s, 3H); MS (ESI) m/z: 386.4 [M+H]⁺. Compounds 20-B and 20-C are atropisomers, and the shown stereochemistry may be the opposite of that depicted.

### Example 21

### (S)-3-(4-Amino-6-(cyclopropyl(methyl)amino)pyrido[3,4-d]pyrimidin-8-yl)-2,4-dimethylphenol (21-B) and (R)-3-(4-amino-6-(cyclopropyl(methyl)amino)pyrido[3,4-d]pyrimidin-8-yl)-2,4-dimethylphenol (21-C)

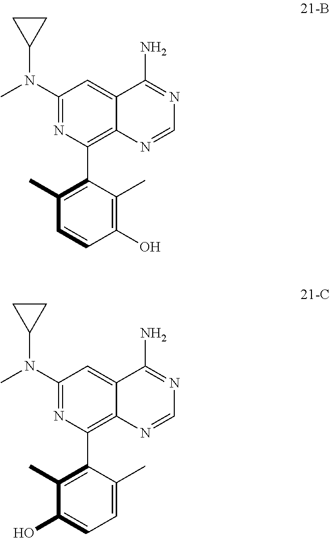

The racemic mixture 3-(4-amino-6-(cyclopropyl(methyl)amino)pyrido[3,4-d]pyrimidin-8-yl)-2,4-dimethylphenol (21-A) (0.15 g) was prepared following the procedure described in Example 1 and was subjected to chiral SFC purification (Chiralpak IH, 30×250 mm column, 35% IPA with 0.1% PrNH₂) to afford the title compounds. From Peak 1: (S)-3-(4-amino-6-(cyclopropyl(methyl)amino)pyrido[3,4-d]pyrimidin-8-yl)-2,4-dimethylphenol (21-B) (30.04 mg, 21%). Chiral purity 99.9% (Chiralpak IH, (4.6×250 mm column), 30% IPA with 0.2% ⁱPrNH₂); ¹H-NMR (400 MHz, DMSO-d₆): δ ppm 9.02 (s, 1H), 8.06 (s, 1H), 7.81 (br. s, 2H), 7.32 (s, 1H), 6.85 (d, J=8.0 Hz, 1H), 6.73 (d, J=8.0 Hz, 1H), 3.08 (s, 3H), 2.56-2.53 (m, 1H), 1.72 (s, 3H), 1.63 (s, 3H), 0.99-0.97 (m, 2H), 0.63-0.61 (m, 2H); MS (ESI) m/z 336.2 [M+H]⁺. From Peak 2: (R)-3-(4-amino-6-(cyclopropyl(methyl)amino)pyrido[3,4-d]pyrimidin-8-yl)-2,4-dimethylphenol (21-C) (27.52 mg, 19%). Chiral purity 98.5% (Chiralpak IH (4.6×250 mm column), 30% IPA with 0.2% PrNH₂); ¹H-NMR (400 MHz, DMSO-d₆): δ ppm 9.03 (s, 1H), 8.06 (s, 1H), 7.81 (br. s, 2H), 7.32 (s, 1H), 6.85 (d, J=8.4 Hz, 1H), 6.73 (d, J=8.0 Hz, 1H), 3.08 (s, 3H), 2.56-2.53 (m, 1H), 1.72 (s, 3H), 1.63 (s, 3H), 1.00-0.96 (m, 2H), 0.63-0.62 (m, 2H); MS (ESI) m/z: 336.2 [M+H]⁺. Compounds 21-B and 21-C are atropisomers, and the shown stereochemistry may be the opposite of that depicted.

### Example 22

### (S)-3-(4-amino-6-(3-(hydroxymethyl)-3-methylazetidin-1-yl) pyrido[3,4-d]pyrimidin-8-yl)-2,4-dimethylphenol (22-B) and (R)-3-(4-amino-6-(3-(hydroxymethyl)-3-methylazetidin-1-yl) pyrido[3,4-d]pyrimidin-8-yl)-2,4-dimethylphenol (22-C)

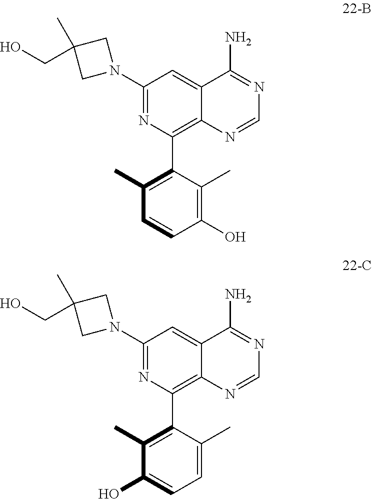

The racemic mixture of 3-(4-amino-6-(3-(hydroxymethyl)-3-methylazetidin-1-yl) pyrido[3,4-d]pyrimidin-8-yl)-2,4-dimethylphenol (22-A) (0.10 g) was prepared following the procedure described in Example 1 and was subjected to chiral SFC purification (Chiralcel-OJ-H, 30×250 mm column, 25% MeOH) to afford the title compounds. From Peak 1: (S)-3-(4-amino-6-(3-(hydroxymethyl)-3-methylazetidin-1-yl)pyrido[3,4-d]pyrimidin-8-yl)-2,4-dimethylphenol (22-B) (24.9 mg, 25%). Chiral purity 98.7% (Chiralcel OJ-H (4.6×250 mm column), 25% MeOH); ¹H NMR (400 MHz, DMSO-d₆): δ ppm 9.03 (s, 1H), 8.03 (s, 1H), 7.73 (br. s, 2H), 6.87 (s, 1H), 6.84 (d, J=8.0 Hz, 1H), 6.73 (d, J=8.0 Hz, 1H), 4.90 (t, J=5.4 Hz, 1H), 3.84-3.78 (m, 2H), 3.53 (d, J=7.6 Hz, 2H), 3.44 (d, J=5.2 Hz, 2H), 1.71 (s, 3H), 1.62 (s, 3H), 1.27 (s, 3H); MS (ESI) m/z: 366.4 [M+H]⁺. From Peak 2: (R)-3-(4-amino-6-(3-(hydroxymethyl)-3-methylazetidin-1-yl)pyrido[3,4-d]pyrimidin-8-yl)-2,4-dimethylphenol (22-C): (28.55 mg, 29%). Chiral purity 96.4% (Chiralcel OJ-H (4.6×250 mm column), 25% MeOH); ¹H NMR (400 MHz, DMSO-d₆): δ ppm 9.02 (s, 1H), 8.03 (s, 1H), 7.73 (br. s, 2H), 6.87 (s, 1H), 6.84 (d, J=8.0 Hz, 1H), 6.73 (d, J=8.0 Hz, 1H), 4.90 (t, J=5.4 Hz, 1H), 3.83-3.79 (m, 2H), 3.53 (d, J=7.2 Hz, 2H), 3.44 (d, J=5.2 Hz, 2H), 1.71 (s, 3H), 1.62 (s, 3H), 1.27 (s, 3H); MS (ESI) m/z: 366.4 [M+H]⁺. Compounds 22-B and 22-C are atropisomers, and the shown stereochemistry may be the opposite of that depicted.

### Example 23

### 3-((S)-4-amino-6-((S)-3-(methoxymethyl)pyrrolidin-1-yl)pyrido[3,4-d]pyrimidin-8-yl)-2,4-dimethylphenol (23-B) and 3-((R)-4-amino-6-((S)-3-(methoxymethyl)pyrrolidin-1-yl)pyrido[3,4-d]pyrimidin-8-yl)-2,4-dimethylphenol (23-C)

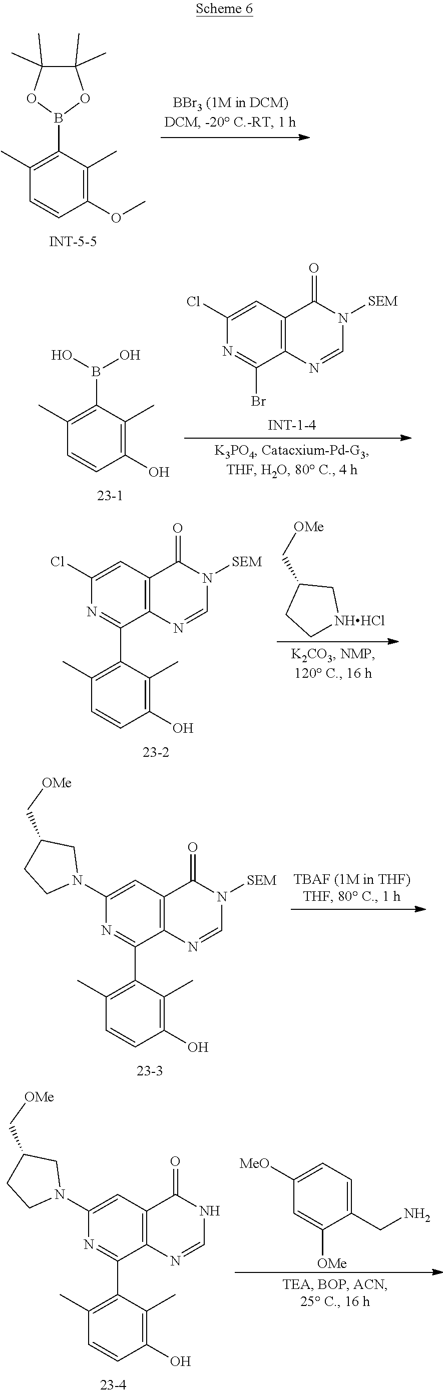

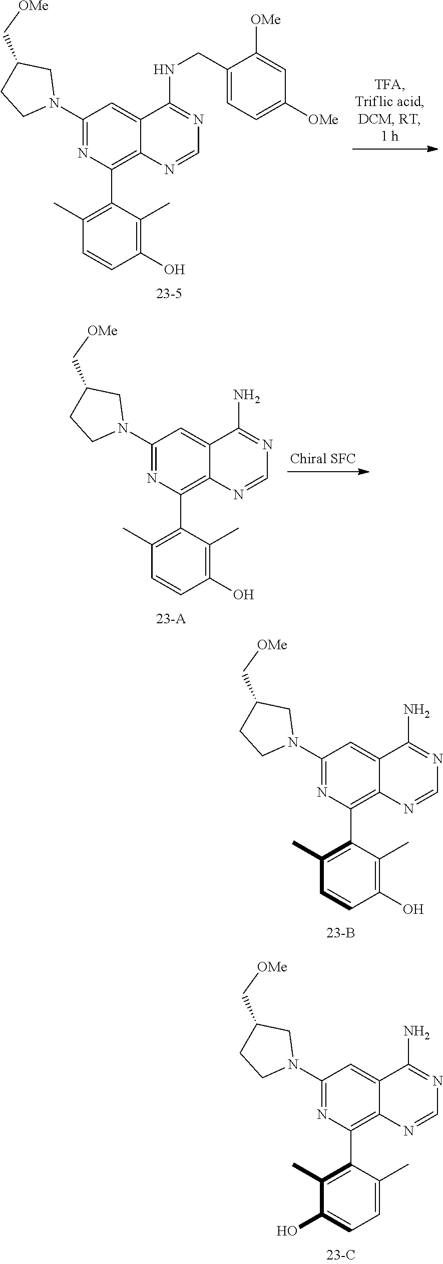

### (3-hydroxy-2,6-dimethylphenyl)boronic acid (23-1)

To a solution of 2-(3-methoxy-2,6-dimethylphenyl)-4,4,5,5-tetramethyl-1,3,2-dioxaborolane (1.20 g, 4.58 mmol) in DCM (12 mL) was added BBr₃ (1M in DCM, 13.73 mL, 13.73 mmol) dropwise at −20° C. and the mixture was stirred at rt for 1 h. After completion, the reaction was quenched with sat. NaHCO₃ (~30 mL) and extracted with EtOAc (2×30 mL). The combined organic layers were washed with brine (30 mL), dried over Na₂SO₄, filtered, and concentrated. The residue was purified by column chromatography (SiO₂, EtOAc/PET) to afford (3-hydroxy-2,6-dimethylphenyl)boronic acid (23-1) (0.45 g, 59%). MS (ESI) m/z: 167.2 [M+H]⁺.

### 6-chloro-8-(3-hydroxy-2,6-dimethylphenyl)-3-((2-(trimethylsilyl)ethoxy)methyl)pyrido[3,4-d]pyrimidin-4 (3H)-one (23-2)

To a solution of 8-bromo-6-chloro-3-((2-(trimethylsilyl)ethoxy)methyl)pyrido[3,4-d]pyrimidin-4 (3H)-one (1.0 g, 2.56 mmol), (3-hydroxy-2,6-dimethylphenyl)boronic acid (23-1) (0.43 g, 2.56 mmol) in THF (9 mL) and water (1 mL) was added K₃PO₄ (1.09 g, 5.12 mmol) and degassed with Argon for 3 min. followed by addition of cataCXium-A-Pd-G₃ (0.19 g, 0.26 mmol). The mixture was stirred at 80° C. for 4 h. After completion, water (30 mL) was added and extracted with EtOAc (2×30 mL). The combined organic layers were washed with brine (20 mL), dried over Na₂SO₄, filtered, and concentrated. The residue was purified by column chromatography (SiO₂, EtOAc/PET) to afford 6-chloro-8-(3-hydroxy-2,6-dimethylphenyl)-3-((2-(trimethylsilyl)ethoxy)methyl)pyrido[3,4-d]pyrimidin-4 (3H)-one (23-2) (0.50 g, 45%). MS (ESI) m/z: 432.7 [M+H]⁺.

### 8-(3-hydroxy-2,6-dimethylphenyl)-6-((S)-3-(methoxymethyl)pyrrolidin-1-yl)-3-((2-(trimethylsilyl)ethoxy)methyl)pyrido[3,4-d]pyrimidin-4 (3H)-one (23-3)

To a solution of 6-chloro-8-(3-hydroxy-2,6-dimethylphenyl)-3-((2-(trimethylsilyl)ethoxy)methyl)pyrido[3,4-d]pyrimidin-4 (3H)-one (23-2) (0.40 g, 0.93 mmol) in NMP (2 mL) was added K₂CO₃ (1.02 g, 7.41 mmol) and (S)-3-(methoxymethyl)pyrrolidine (0.32 g, 2.78 mmol) at rt then heated at 120° C. for 16 h. After completion, water (20 mL) was added and extracted with EtOAc (2×30 mL). The combined organic layers were washed with brine (30 mL), dried over Na₂SO₄, filtered, and concentrated. The residue was purified by column chromatography (SiO₂, EtOAc/PET) to afford 8-(3-hydroxy-2,6-dimethylphenyl)-6-((S)-3-(methoxymethyl)pyrrolidin-1-yl)-3-((2-(trimethylsilyl)ethoxy)methyl)pyrido[3,4-d]pyrimidin-4 (3H)-one (23-3) (0.40 g, 84%). MS (ESI) m/z: 511.5 [M+H]⁺.

### 8-(3-hydroxy-2,6-dimethylphenyl)-6-((S)-3-(methoxymethyl)pyrrolidin-1-yl)pyrido[3,4-d]pyrimidin-4 (3H)-one (23-4)

To a solution of 8-(3-hydroxy-2,6-dimethylphenyl)-6-((S)-3-(methoxymethyl)pyrrolidin-1-yl)-3-((2-(trimethylsilyl)ethoxy)methyl)pyrido[3,4-d]pyrimidin-4 (3H)-one (23-3) (0.40 g, 0.78 mmol) in THF (8 mL) was added TBAF (1M in THF, 3.92 mL, 3.92 mmol) at rt. The solution was heated at 80° C. for 1 h. After completion, water (20 mL) was added and extracted with EtOAc (2×30 mL). The combined organic layers were washed with brine (20 mL), dried over Na₂SO₄, filtered, and concentrated. The residue was purified by column chromatography (SiO₂, EtOAc/PET) to afford 8-(3-hydroxy-2,6-dimethylphenyl)-6-((S)-3-(methoxymethyl)pyrrolidin-1-yl)pyrido[3,4-d]pyrimidin-4 (3H)-one (23-4) (0.22 g, 73%). MS (ESI) m/z: 381.4 [M+H]⁺.

### 3-(4-((2,4-dimethoxybenzyl)amino)-6-((S)-3-(methoxymethyl)pyrrolidin-1-yl)pyrido[3,4-d]pyrimidin-8-yl)-2,4-dimethylphenol (23-5)

To a solution of 8-(3-hydroxy-2,6-dimethylphenyl)-6-((S)-3-(methoxymethyl)pyrrolidin-1-yl)pyrido[3,4-d]pyrimidin-4 (3H)-one (23-4) (0.21 g, 0.55 mmol) in ACN (4.2 mL) were added (2,4-dimethoxyphenyl)methanamine (0.14 g, 0.83 mmol), Et₃N (0.12 mL, 0.83 mmol), BOP (0.32 g, 0.72 mmol) at rt then stirred for 16 h. After completion, water (20 mL) was added and extracted with EtOAc (2×30 mL). The combined organic layers were washed with brine (20 mL), dried over Na₂SO₄, filtered, and concentrated. The residue was purified by column chromatography (SiO₂, EtOAc/PET) to afford 3-(4-((2,4-dimethoxybenzyl)amino)-6-((S)-3-(methoxymethyl)pyrrolidin-1-yl)pyrido[3,4-d]pyrimidin-8-yl)-2,4-dimethylphenol (23-5) (0.12 g, 41%). MS (ESI) m/z: 530.9 [M+H]⁺.

### 3-(4-amino-6-((S)-3-(methoxymethyl)pyrrolidin-1-yl)pyrido[3,4-d]pyrimidin-8-yl)-2,4-dimethylphenol (23-A)

To a stirred solution of 3-(4-((2,4-dimethoxybenzyl)amino)-6-((S)-3-(methoxymethyl)pyrrolidin-1-yl)pyrido[3,4-d]pyrimidin-8-yl)-2,4-dimethylphenol (23-5) (0.11 g, 0.21 mmol) in DCM (1.1 mL) were added TFA (0.238 mL, 3.115 mmol), TfOH (0.18 mL, 2.08 mmol) at rt then stirred for 1 h. After completion, the reaction was quenched with sat. NaHCO₃ (~20 mL) and extracted with EtOAc (3×20 mL). The combined organic layers were washed with brine (30 mL), dried over Na₂SO₄, filtered, and concentrated. The residue was purified by prep-HPLC (C18, ACN/H₂O with 10 mM NH₄HCO₃) to afford a racemic 3-(4-amino-6-((S)-3-(methoxymethyl)pyrrolidin-1-yl)pyrido[3,4-d]pyrimidin-8-yl)-2,4-dimethylphenol (23-A) (0.12 g, 62%). MS (ESI) m/z: 380.3 [M+H]⁺.

### Isolation of 3-((S)-4-amino-6-((S)-3-(methoxymethyl)pyrrolidin-1-yl)pyrido[3,4-d]pyrimidin-8-yl)-2,4-dimethylphenol (23-B) and 3-((R)-4-amino-6-((S)-3-(methoxymethyl)pyrrolidin-1-yl)pyrido[3,4-d]pyrimidin-8-yl)-2,4-dimethylphenol (23-C)

The racemic mixture 3-(4-amino-6-((S)-3-(methoxymethyl)pyrrolidin-1-yl)pyrido[3,4-d]pyrimidin-8-yl)-2,4-dimethylphenol (23-A) (25 mg) was subjected to chiral SFC purification (Lux Cellulose-4, 30×250 mm column, 25% MeOH) to afford the title compounds. From Peak 1: 3-((S)-4-amino-6-((S)-3-(methoxymethyl)pyrrolidin-1-yl)pyrido[3,4-d]pyrimidin-8-yl)-2,4-dimethylphenol (23-B) (9.3 mg, 48%). Chiral purity 99.8% (Lux Cellulose-4 (4.6×150 mm column) 25% MeOH); ¹H NMR (400 MHz, DMSO-d₆): δ ppm 9.03 (s, 1H), 8.00 (s, 1H), 7.71 (br. s, 2H), 6.89 (s, 1H), 6.84 (d, J=8.0 Hz, 1H), 6.73 (d, J=8.0 Hz, 1H), 3.60-3.50 (m, 2H), 3.47-3.41 (m, 3H), 3.28 (s, 3H), 3.19-3.13 (m, 1H), 2.65-2.57 (m, 1H), 2.13-2.04 (m, 1H), 1.79-1.70 (m, 4H), 1.63 (s, 3H). MS (ESI) m/z: 380.3 [M+H]⁺. From Peak 2: 3-((R)-4-amino-6-((S)-3-(methoxymethyl)pyrrolidin-1-yl)pyrido[3,4-d]pyrimidin-8-yl)-2,4-dimethylphenol (23-C) (9.9 mg, 44%). Chiral purity 96.7% (Lux Cellulose-4 (4.6×150 mm column), 25% MeOH); ¹H-NMR (400 MHz, DMSO-d₆): δ ppm 9.06 (s, 1H), 8.00 (s, 1H), 7.71 (br. s, 2H), 6.89 (s, 1H), 6.84 (d, J=8.4 Hz, 1H), 6.73 (d, J=8.0 Hz, 1H), 3.60-3.50 (m, 2H), 3.46-3.41 (m, 3H), 3.28 (s, 3H), 3.19-3.13 (m, 1H), 2.64-2.57 (m, 1H), 2.13-2.04 (m, 1H), 1.79-1.70 (m, 4H), 1.63 (s, 3H). MS (ESI) m/z: 380.3 [M+H]⁺. Compounds 23-B and 23-C are atropisomers, and the shown stereochemistry may be the opposite of that depicted.

### Example 24

### 3-((S)-4-Amino-6-((S)-2-(methoxymethyl)azetidin-1-yl)pyrido[3,4-d]pyrimidin-8-yl)-2,4-dimethylphenol (24-B) and 3-((R)-4-amino-6-((S)-2-(methoxymethyl)azetidin-1-yl)pyrido[3,4-d]pyrimidin-8-yl)-2,4-dimethylphenol (24-C)

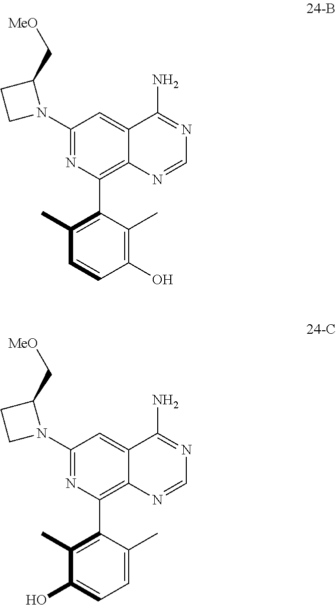

The racemic mixture 3-(4-amino-6-((S)-2-(methoxymethyl)azetidin-1-yl)pyrido[3,4-d]pyrimidin-8-yl)-2,4-dimethylphenol (24-A) (0.07 g) was prepared following the procedure described in Example 23 (replacing K₂CO₃ with DIPEA in DMSO in step 3) and was separated by chiral SFC (Lux Cellulose-4, 30×250 mm column, 30% MeOH with 0.1% 7N NH₃ in MeOH) to afford the title compounds. From Peak 1: 3-((S)-4-amino-6-((S)-2-(methoxymethyl)azetidin-1-yl)pyrido[3,4-d]pyrimidin-8-yl)-2,4-dimethylphenol (24-B) (7.17 mg, 10%). Chiral purity 99.9% (Lux Cellulose-4 (4.6×250 mm column), 30% MeOH with 0.5% DEA); ¹H-NMR (400 MHz, DMSO-d₆): δ ppm 9.04 (s, 1H), 8.05 (s, 1H), 7.76 (br. s, 2H), 6.89 (s, 1H), 6.84 (d, J=8.0 Hz, 1H), 6.73 (d, J=8.4 Hz, 1H), 4.38-4.30 (m, 1H), 3.95-3.88 (m, 1H), 3.83-3.77 (m, 1H), 3.72-3.68 (m, 2H), 3.30 (s, 3H), 2.43-2.27 (m, 2H), 1.70 (s, 3H), 1.64 (s, 3H). MS (ESI) m/z: 366.4 [M+H]⁺. From Peak 2: 3-((R)-4-amino-6-((S)-2-(methoxymethyl)azetidin-1-yl)pyrido[3,4-d]pyrimidin-8-yl)-2,4-dimethylphenol (24-C): (5.05 mg, 7%). Chiral purity 99.0% (Lux Cellulose-4 (4.6×250 mm column), 30% MeOH with 0.5% DEA); ¹H-NMR (400 MHz, DMSO-d₆): δ ppm 9.04 (s, 1H), 8.05 (s, 1H), 7.76 (br. s, 2H), 6.89 (s, 1H), 6.86 (d, J=8.0 Hz, 1H), 6.73 (d, J=8.0 Hz, 1H), 4.38-4.30 (m, 1H), 3.96-3.88 (m, 1H), 3.84-3.77 (m, 1H), 3.69 (d, J=4.8 Hz, 2H), 3.30 (s, 3H), 2.44-2.27 (m, 2H), 1.73 (s, 3H), 1.61 (s, 3H). MS (ESI) m/z: 366.4 [M+H]⁺. Compounds 24-B and 24-C are atropisomers, and the shown stereochemistry may be the opposite of that depicted.

### Example 25

### 3-((S)-4-Amino-6-((R)-2-(methoxymethyl)azetidin-1-yl)pyrido[3,4-d]pyrimidin-8-yl)-2,4-dimethylphenol (25-B) and 3-((R)-4-amino-6-((R)-2-(methoxymethyl)azetidin-1-yl)pyrido[3,4-d]pyrimidin-8-yl)-2,4-dimethylphenol (25-C)

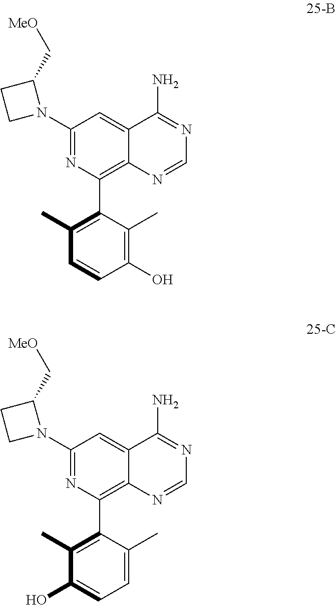

The racemic mixture of 3-(4-amino-6-((R)-2-(methoxymethyl)azetidin-1-yl)pyrido[3,4-d]pyrimidin-8-yl)-2,4-dimethylphenol (25-A) (34 mg) was prepared following the procedure described in Example 24 and was subjected to chiral SFC purification (Chiralpak IC, 30×250 mm column, 30% IPA) to afford the title compounds. From Peak 1: 3-((S)-4-amino-6-((R)-2-(methoxymethyl)azetidin-1-yl)pyrido[3,4-d]pyrimidin-8-yl)-2,4-dimethylphenol (25-1) (10 mg, 4%). Chiral purity 99.9% (Chiralpak IC (4.6×250 mm column), 30% IPA with 0.2% PrNH₂); ¹H-NMR (400 MHz, DMSO-d₆) δ ppm 9.03 (s, 1H), 8.05 (s, 1H), 7.75 (br. s, 2H), 6.89 (s, 1H), 6.84 (d, J=8.0 Hz, 1H), 6.73 (d, J=8.0 Hz, 1H), 4.40-4.30 (m, 1H), 3.97-3.87 (m, 1H), 3.85-3.77 (m, 1H), 3.76-3.65 (m, 2H), 3.30 (s, 3H), 2.40-2.27 (m, 2H), 1.70 (s, 3H), 1.64 (s, 3H); MS (ESI) m/z: 366.4 [M+H]⁺. From Peak 2: 3-((R)-4-amino-6-((R)-2-(methoxymethyl)azetidin-1-yl)pyrido[3,4-d]pyrimidin-8-yl)-2,4-dimethylphenol (25-2): (13 mg, 5%). Chiral purity 95.1% (Chiralpak IC (4.6×250 mm column), 30% IPA with 0.2% PrNH₂); ¹H-NMR (400 MHz, DMSO-d₆) δ ppm 9.02 (s, 1H), 8.04 (s, 1H), 7.75 (br. s, 2H), 6.89 (s, 1H), 6.85 (d, J=8.0 Hz, 1H), 6.73 (d, J=8.0 Hz, 1H), 4.40-4.30 (m, 1H), 3.98-3.89 (m, 1H), 3.87-3.78 (m, 1H), 3.69 (d, J=4.4 Hz, 2H), 3.30 (s, 3H), 2.45-2.25 (m, 2H), 1.73 (s, 3H), 1.61 (s, 3H); MS (ESI) m/z: 366.4 [M+H]⁺. Compounds 25-1 and 25-2 are atropisomers, and the shown stereochemistry may be the opposite of that depicted.

### Example 26

### (S)-3-(4-Amino-6-(3-methoxyazetidin-1-yl)pyrido[3,4-d]pyrimidin-8-yl)-2,4-dimethylphenol (26-B) and (R)-3-(4-amino-6-(3-methoxyazetidin-1-yl)pyrido[3,4-d]pyrimidin-8-yl)-2,4-dimethylphenol (26-C)

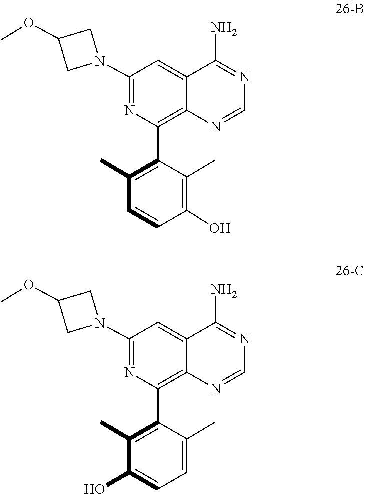

The racemic mixture of 3-(4-amino-6-(3-methoxyazetidin-1-yl)pyrido[3,4-d]pyrimidin-8-yl)-2,4-dimethylphenol (22 mg) was prepared following the procedure described in Example 2 (replacing Cs₂CO₃ with NaOtBu and Xphos with BINAP in 1,4-dioxane in the first step) and was subjected to chiral SFC purification (Chiralcel OJ-H, 30×250 mm column, 20% MeOH with 0.1% 7N NH₃ in MeOH) to afford the title compounds. From Peak 1: (S)-3-(4-amino-6-(3-methoxyazetidin-1-yl)pyrido[3,4-d]pyrimidin-8-yl)-2,4-dimethylphenol (26-1) (3.07 mg, 14%). Chiral purity 99.4% (Chiralcel OJ-H (4.6×250 mm column), 20% MeOH with 0.5% DEA); ¹H NMR (400 MHz, DMSO-d₆): δ ppm 9.07 (s, 1H), 8.08 (s, 1H), 7.83 (br. s, 2H), 6.98 (s, 1H), 6.86 (d, J=8.0 Hz, 1H), 6.75 (d, J=8.0 Hz, 1H), 4.40-4.33 (m, 1H), 4.21-4.15 (m, 2H), 3.80-3.74 (m, 2H), 3.26 (s, 3H), 1.71 (s, 3H), 1.62 (s, 3H). MS (ESI) m/z: 352.3 [M+H]⁺. From Peak 2: (R)-3-(4-amino-6-(3-methoxyazetidin-1-yl)pyrido[3,4-d]pyrimidin-8-yl)-2,4-dimethylphenol (26-2) (2 mg, 9%). Chiral purity 99.2% (Chiralcel OJ-H (4.6×250 mm column), 20% MeOH with 0.5% DEA); ¹H NMR (400 MHz, DMSO-d₆): δ ppm 9.11 (s, 1H), 8.11 (s, 1H), 7.88 (br. s, 2H), 7.01 (s, 1H), 6.88 (d, J=8.0 Hz, 1H), 6.76 (d, J=8.0 Hz, 1H), 4.40-4.33 (m, 1H), 4.24-4.15 (m, 2H), 3.83-3.73 (m, 2H), 3.26 (s, 3H), 1.72 (s, 3H), 1.63 (s, 3H). MS (ESI) m/z: 352.3 [M+H]⁺. Compounds 26-B and 26-C are atropisomers, and the shown stereochemistry may be the opposite of that depicted.

### Example 27

### (S)-3-(4-Amino-6-(ethyl(methyl)amino)pyrido[3,4-d]pyrimidin-8-yl)-2,4-dimethylphenol (27-B) and (R)-3-(4-amino-6-(ethyl(methyl)amino)pyrido[3,4-d]pyrimidin-8-yl)-2,4-dimethylphenol (27-C)

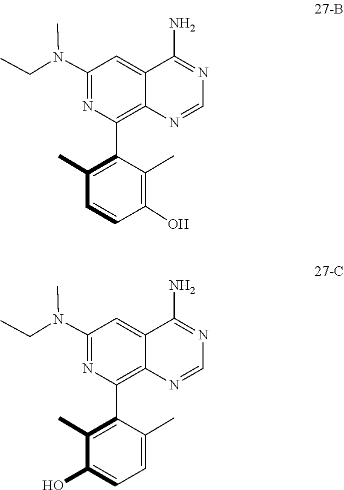

The racemic mixture of 3-(4-amino-6-(ethyl(methyl)amino)pyrido[3,4-d]pyrimidin-8-yl)-2,4-dimethylphenol (27-A) (0.13 g) was prepared following the procedure described in Example 1 and was subjected to chiral SFC purification (Chiralpak IC, 15% ACN/MeOH (1:1) with 0.2% 7N NH₃ in MeOH) to afford the title compounds. From Peak 1: (S)-3-(4-amino-6-(ethyl(methyl)amino)pyrido[3,4-d]pyrimidin-8-yl)-2,4-dimethylphenol (27-B): (6 mg, 5%). Chiral purity 94.7% (Chiralcel OJ-H (4.6×250 mm column), 15% ACN/MeOH (1:1) with 0.2% 7N NH₃ in MeOH); ¹H NMR (400 MHz, DMSO-d₆): δ ppm 8.67 (br. s, 1H), 8.01 (s, 1H), 7.73 (br. s, 2H), 7.00 (s, 1H), 6.85 (d, J=8.0 Hz, 1H), 6.73 (d, J=8.0 Hz, 1H), 3.66-3.58 (m, 2H), 3.03 (s, 3H), 1.72 (s, 3H), 1.63 (s, 3H), 1.05 (t, J=7.0 Hz, 3H); MS (ESI) m/z: 324.3 [M+H]⁺. From Peak 2: (R)-3-(4-amino-6-(ethyl(methyl)amino)pyrido[3,4-d]pyrimidin-8-yl)-2,4-dimethylphenol (27-C): (7 mg g, 5%). Chiral purity 85.7% (Chiralcel OJ-H (4.6×250 mm column), 15% ACN/MeOH (1:1) with 0.2% 7N NH₃ in MeOH); ¹H NMR (400 MHz, DMSO-d₆): δ ppm 8.73 (br. s, 1H), 8.01 (s, 1H), 7.73 (br. s, 2H), 7.00 (s, 1H), 6.85 (d, J=8.0 Hz, 1H), 6.73 (d, J=8.4 Hz, 1H), 3.64-3.59 (m, 2H), 3.03 (s, 3H), 1.72 (s, 3H), 1.63 (s, 3H), 1.06 (t, J=7.0 Hz, 3H); MS (ESI) m/z: 324.3 [M+H]⁺. Compounds 27-B and 27-C are atropisomers, and the shown stereochemistry may be the opposite of that depicted.

### Example 28

### (S)-3-(4-Amino-6-((2,2-difluoroethyl)(methyl)amino)pyrido[3,4-d]pyrimidin-8-yl)-2,4-dimethylphenol (28-B) and (R)-3-(4-amino-6-((2,2-difluoroethyl)(methyl)amino)pyrido[3,4-d]pyrimidin-8-yl)-2,4-dimethylphenol (28-C)

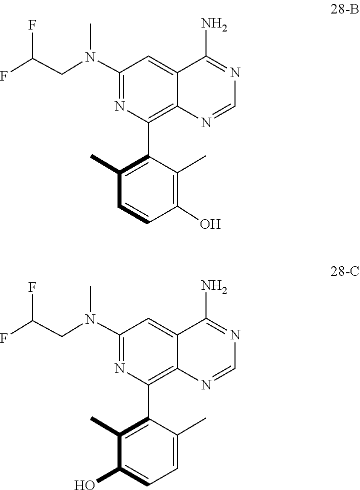

The racemic mixture of 3-(4-amino-6-((2,2-difluoroethyl)(methyl)amino)pyrido[3,4-d]pyrimidin-8-yl)-2,4-dimethylphenol (28-A) (0.10 g) was prepared following the procedure described in Example 2 (replacing Pd₂(dba)₃/Xphos with Pd-PEPPSI-iPent-Cl in 1,4-dioxane in the first step) and was subjected to chiral SFC purification (Chiralcel OJ-H, 30×250 mm column, 10% ACN/MeOH (1:1) with 0.2% 7N NH₃ in MeOH) to afford the title compounds. From Peak 1: (S)-3-(4-amino-6-((2,2-difluoroethyl)(methyl)amino)pyrido[3,4-d]pyrimidin-8-yl)-2,4-dimethylphenol (28-B) (32 mg, 32%). Chiral purity 99.8% (Chiralcel OJ-H (4.6×250 mm column), 10% ACN/MeOH (1:1) with 0.2% 7N NH₃ in MeOH); ¹H NMR (400 MHz, DMSO-d₆) δ ppm 9.05 (s, 1H), 8.07 (s, 1H), 7.82 (br. s 2H), 7.15 (s, 1H), 6.85 (d, J=8.0 Hz, 1H), 6.74 (d, J=8.0 Hz, 1H), 6.28-5.95 (m, 1H), 4.10-3.97 (m, 2H), 3.15 (s, 3H), 1.71 (s, 3H), 1.62 (s, 3H); MS (ESI) m/z: 360.3 [M+H]⁺. From Peak 2: (R)-3-(4-amino-6-((2,2-difluoroethyl)(methyl)amino)pyrido[3,4-d]pyrimidin-8-yl)-2,4-dimethylphenol (28-C) (35 mg, 35%). Chiral purity 97.4% (Chiralcel OJ-H (4.6×250 mm column), 10% ACN/MeOH (1:1) with 0.2% 7N NH₃ in MeOH); ¹H NMR (400 MHz, DMSO-d₆) δ ppm 9.05 (s, 1H), 8.07 (s, 1H), 7.82 (br. s 2H), 7.15 (s, 1H), 6.85 (d, J=8.0 Hz, 1H), 6.74 (d, J=8.0 Hz, 1H), 6.28-5.95 (m, 1H), 4.10-3.96 (m, 2H), 3.15 (s, 3H), 1.71 (s, 3H), 1.62 (s, 3H); MS (ESI) m/z: 360.3 [M+H]⁺. Compounds 28-B and 28-C are atropisomers, and the shown stereochemistry may be the opposite of that depicted.

### Example 29

### (S)-3-(4-Amino-6-(methyl(2,2,2-trifluoroethyl)amino)pyrido[3,4-d]pyrimidin-8-yl)-2,4-dimethylphenol (29-B) and (R)-3-(4-amino-6-(methyl(2,2,2-trifluoroethyl)amino)pyrido[3,4-d]pyrimidin-8-yl)-2,4-dimethylphenol (29-C)

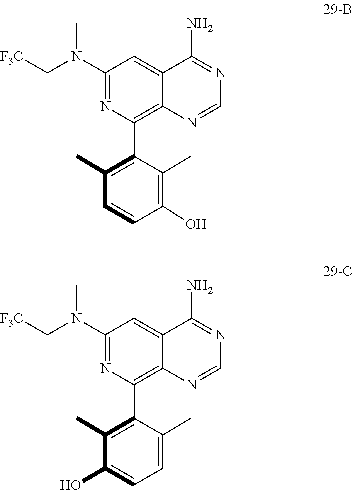

The racemic mixture of 3-(4-amino-6-(methyl(2,2,2-trifluoroethyl)amino)pyrido[3,4-d]pyrimidin-8-yl)-2,4-dimethylphenol (29-A) (0.19 g) was prepared following the procedure described in Example 28 (replacing Cs₂CO₃ with K₃PO₄ in the first step) and was subjected to chiral SFC purification (Chiracel OJ-H, 12% ACN:MeOH (1:1) with 0.3% 7N NH₃ in MeOH) to afford the title compounds. From Peak 1: (S)-3-(4-amino-6-(methyl(2,2,2-trifluoroethyl)amino)pyrido[3,4-d]pyrimidin-8-yl)-2,4-dimethylphenol (29-B) (79.99 mg, 42%). Chiral purity 99.6% (Chiracel OJ-H (4.6×250 mm column), 10% ACN/MeOH (1:1) with 0.2% 7N NH₃ in MeOH); ¹H NMR (400 MHz, DMSO-d₆): δ ppm 9.07 (s, 1H), 8.10 (s, 1H), 7.88 (br. s, 2H), 7.23 (s, 1H), 6.86 (d, J=8.0 Hz, 1H), 6.75 (d, J=8.0 Hz, 1H), 4.59-4.45 (m, 2H), 3.18 (s, 3H), 1.69 (s, 3H), 1.61 (s, 3H). MS (ESI) m/z: 378.3 [M+H]⁺. From Peak 2: (R)-3-(4-amino-6-(methyl(2,2,2-trifluoroethyl)amino)pyrido[3,4-d]pyrimidin-8-yl)-2,4-dimethylphenol (29-C) (75.48 mg, 40%). Chiral purity 97.3% (Chiracel OJ-H (4.6×250 mm column), 10% ACN/MeOH (1:1) with 0.2% 7N NH₃ in MeOH); ¹H NMR (400 MHz, DMSO-d₆): δ ppm 9.06 (s, 1H), 8.09 (s, 1H), 7.85 (br. s, 2H), 7.22 (s, 1H), 6.86 (d, J=8.4 Hz, 1H), 6.74 (d, J=8.0 Hz, 1H), 4.58-4.45 (m, 2H), 3.17 (s, 3H), 1.69 (s, 3H), 1.60 (s, 3H). MS (ESI) m/z: 378.2 [M+H]⁺. Compounds 29-B and 29-C are atropisomers, and the shown stereochemistry may be the opposite of that depicted.

### Example 30

### (S)-3-(4-amino-6-((cyclopropylmethyl)(methyl)amino)pyrido[3,4-d]pyrimidin-8-yl)-2,4-dimethylphenol (30-B) and (R)-3-(4-amino-6-((cyclopropylmethyl)(methyl)amino)pyrido[3,4-d]pyrimidin-8-yl)-2,4-dimethylphenol (30-C)

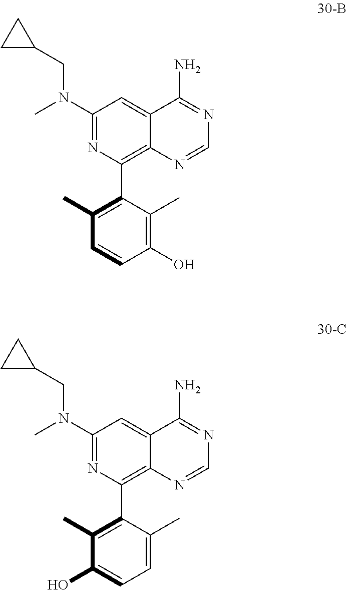

The racemic mixture of 3-(4-amino-6-((cyclopropylmethyl)(methyl)amino)pyrido[3,4-d]pyrimidin-8-yl)-2,4-dimethylphenol (30-A) (0.20 g) was prepared following the procedure described in Example 1 and was subjected to chiral SFC purification (Chiralpak-OJ-H, 25% ACN/IPA (1:1) with 0.1% 7N NH₃ in MeOH) to afford the tittle compounds. From Peak 1: (S)-3-(4-amino-6-((cyclopropylmethyl)(methyl)amino)pyrido[3,4-d]pyrimidin-8-yl)-2,4-dimethylphenol (30-B) (45 mg, 17%). Chiral purity 98.8% (Chiralcel OJ-H (4.6×250 mm column), 30% ACN/ⁱPrOH (1:1) with 0.2% DEA); ¹H NMR (400 MHz, DMSO-d₆) δ ppm 9.03 (s, 1H), 8.01 (s, 1H), 7.74 (br. s, 2H), 7.01 (s, 1H), 6.85 (d, J=8.0 Hz, 1H), 6.73 (d, J=8.0 Hz, 1H), 3.49 (d, J=6.4 Hz, 2H), 3.10 (s, 3H), 1.72 (s, 3H), 1.63 (s, 3H), 1.06-0.97 (m, 1H), 0.45-0.38 (m, 2H), 0.25-0.19 (m, 2H). MS (ESI) m/z: 350.4 [M+H]⁺. From Peak 2: (R)-3-(4-amino-6-((cyclopropylmethyl)(methyl)amino)pyrido[3,4-d]pyrimidin-8-yl)-2,4-dimethylphenol (30-C) (34.7 mg, 13%). Chiral purity 98.1% (Chiralcel OJ-H (4.6×250 mm column), 30% ACN/ⁱPrOH (1:1) with 0.2% DEA); ¹H NMR (400 MHz, DMSO-d₆) δ ppm 9.04 (s, 1H), 8.02 (s, 1H), 7.80 (br. s, 2H), 7.02 (s, 1H), 6.85 (d, J=8.0 Hz, 1H), 6.73 (d, J=8.0 Hz, 1H), 3.49 (d, J=6.8 Hz, 2H), 3.10 (s, 3H), 1.72 (s, 3H), 1.63 (s, 3H), 1.05-0.96 (m, 1H), 0.44-0.38 (m, 2H), 0.24-0.19 (m, 2H). MS (ESI) m/z: 350.4 [M+H]⁺. Compounds 30-B and 30-C are atropisomers, and the shown stereochemistry may be the opposite of that depicted.

### Example 31

### (S)-3-(4-amino-6-(2,4-difluorophenyl)pyrido[3,4-d]pyrimidin-8-yl)-2,4-dimethylphenol (31-B) and (R)-3-(4-amino-6-(2,4-difluorophenyl)pyrido[3,4-d]pyrimidin-8-yl)-2,4-dimethylphenol (31-C)

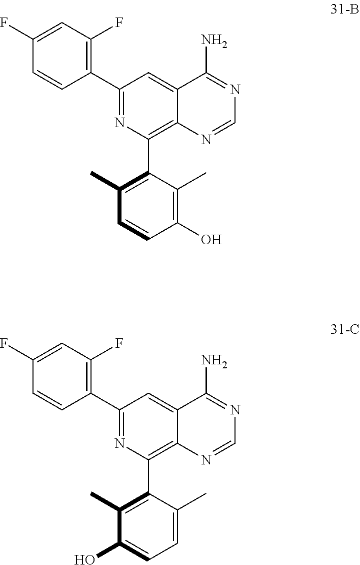

The racemic mixture of 3-(4-amino-6-(2,4-difluorophenyl)pyrido[3,4-d]pyrimidin-8-yl)-2,4-dimethylphenol (31-A) (0.16 g) was prepared following the procedure described in Example 19 and was subjected to chiral SFC (Lux Cellulose-4, 4.6×250 mm column, 20% MeOH) to afford the title compounds. From Peak 1: (S)-3-(4-amino-6-(2,4-difluorophenyl)pyrido[3,4-d]pyrimidin-8-yl)-2,4-dimethylphenol (31-B) (0.05 g, 25%). Chiral purity 99.54% (Lux Cellulose-4, 4.6×250 mm column, 20% MeOH); ¹H NMR (400 MHz, DMSO-d₆): δ ppm 9.11 (s, 1H), 8.49 (s, 1H), 8.39 (s, 1H), 8.38-8.10 (m, 2H), 7.98-7.92 (m, 1H), 7.25-7.22 (m, 1H), 7.20 (t, J=7.2 Hz, 1H), 6.99 (d, J=8 Hz, 1H), 6.78 (d, J=8.0 Hz, 1H), 1.74 (s, 3H), 1.66 (s, 3H). MS (ESI) m/z 379.39 [M+H]⁺. From Peak 2: (R)-3-(4-amino-6-(2,4-difluorophenyl)pyrido[3,4-d]pyrimidin-8-yl)-2,4-dimethylphenol (31-C) (0.055 g, 27%). Chiral purity 95.4% (Lux Cellulose-4, 4.6×250 mm column, 20% MeOH); ¹H NMR (400 MHz, DMSO-d₆): δ ppm 9.11 (s, 1H), 8.49 (s, 1H), 8.39 (s, 1H), 8.38-8.10 (m, 2H), 7.98.7.94 (m, 1H), 7.47-7.41 (m, 1H), 7.26-7.21 (m, 1H), 6.90 (d, J=8.0 Hz, 1H), 6.78 (d, J=8.0 Hz, 1H), 1.74 (s, 3H), 1.65 (s, 3H). MS (ESI) m/z 379.39 [M+H]⁺. Compounds 31-B and 31-C are atropisomers, and the shown stereochemistry may be the opposite of that depicted.

### Example 32

### (S)-3-(4-amino-6-(pyridin-4-yl)pyrido[3,4-d]pyrimidin-8-yl)-2,4-dimethylphenol (32-B) and (R)-3-(4-amino-6-(pyridin-4-yl)pyrido[3,4-d]pyrimidin-8-yl)-2,4-dimethylphenol (32-C)

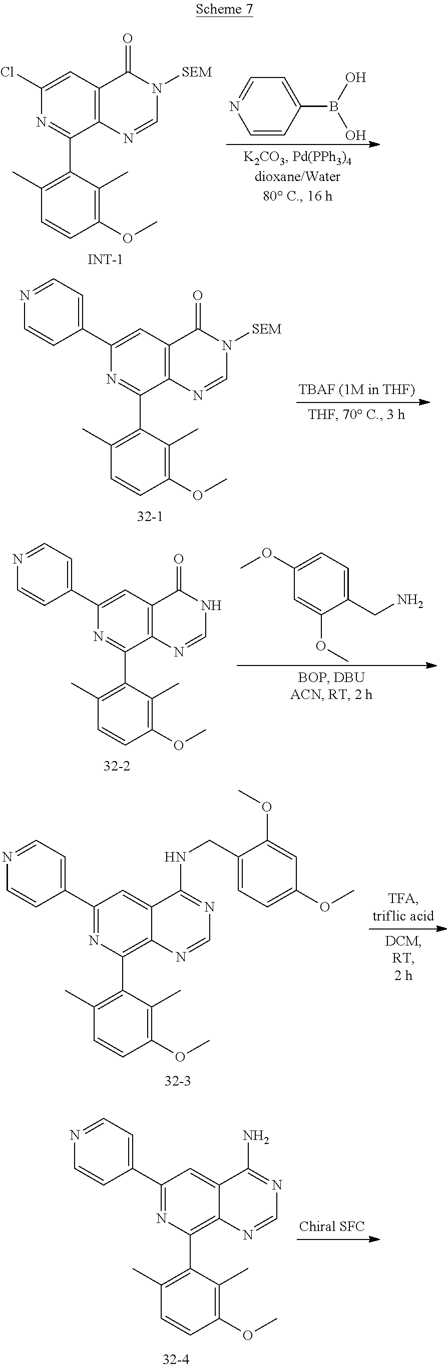

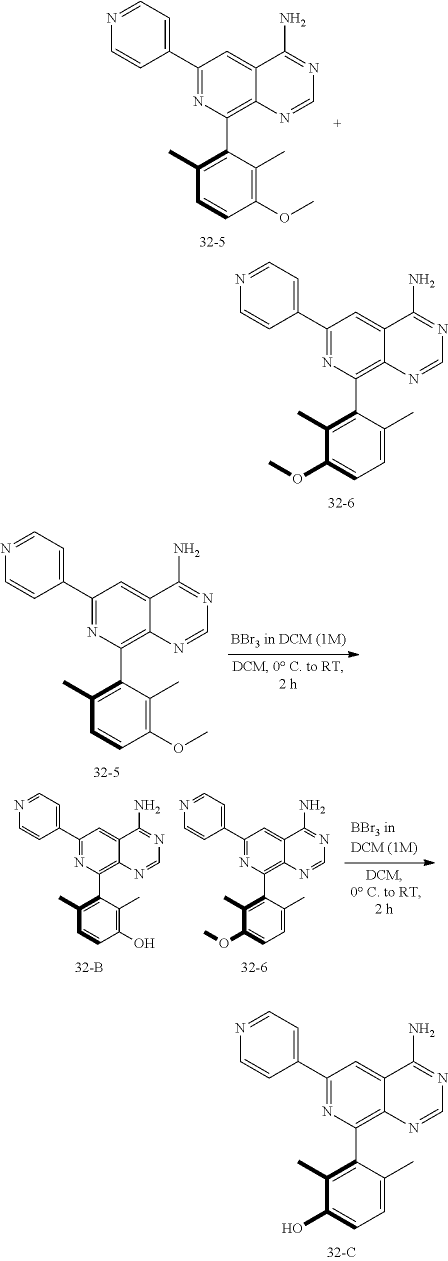

### 8-(3-methoxy-2,6-dimethylphenyl)-6-(1-methyl-1H-pyrazol-4-yl)-3-((2-(trimethylsilyl)ethoxy)methyl)pyrido[3,4-d]pyrimidin-4 (3H)-one (32-1)

To a solution of 6-chloro-8-(3-methoxy-2,6-dimethylphenyl)-3-((2-(trimethylsilyl)ethoxy)methyl)pyrido[3,4-d]pyrimidin-4 (3H)-one (INT-1) (0.8 g, 1.79 mmol) and pyridin-4-ylboronic acid (0.794 g, 6.46 mmol) in a mixture of 1,4-dioxane (26.4 mL) and water (5.6 mL) was added K₂CO₃ (0.744 g, 5.381 mmol), and the mixture was degassed the mixture with N₂ for 5 min. Tetrakis(triphenylphosphine)palladium(0) (0.207 g, 0.179 mmol) was added and the mixture was degassed again with N₂ for 3 minutes. The mixture was stirred at 80° C. for 16 h. After completion, the mixture was diluted with water (100 mL) and extracted with EtOAc (3×100 mL). The filtrate was concentrated under reduced pressure. The residue was purified by column chromatography (SiO₂, EtOAc/PET) to afford 8-(3-methoxy-2,6-dimethylphenyl)-6-(pyridin-4-yl)-3-((2-(trimethylsilyl)ethoxy)methyl)pyrido[3,4-d]pyrimidin-4 (3H)-one (32-1) (0.8 g, 90%). MS (ESI) m/z: 489.68 [M+H]⁺.

### 8-(3-methoxy-2,6-dimethylphenyl)-6-(pyridin-4-yl)pyrido[3,4-d]pyrimidin-4 (3H)-one (32-2)

To a solution of 8-(3-methoxy-2,6-dimethylphenyl)-6-(1-methyl-1H-pyrazol-4-yl)-3-((2-(trimethylsilyl)ethoxy)methyl)pyrido[3,4-d]pyrimidin-4 (3H)-one (32-1) (0.9 g, 1.842 mmol) in THF (18.0 mL) was added TBAF (1M in THF, 7.37 mL, 7.37 mmol). The mixture was stirred at 70° C. for 3 h. After completion, the mixture was diluted with water (10 mL) and extracted with EtOAc (2×10 mL). The filtrate was concentrated under reduced pressure. The residue was purified by column chromatography (SiO₂, EtOAc/PET) to afford 8-(3-methoxy-2,6-dimethylphenyl)-6-(pyridin-4-yl)pyrido[3,4-d]pyrimidin-4 (3H)-one (32-2) (0.75 g, 83%). MS (ESI) m/z: 359.61 [M+H]⁺.

### N-(2,4-dimethoxybenzyl)-8-(3-methoxy-2,6-dimethylphenyl)-6-(pyridin-4-yl)pyrido[3,4-d]pyrimidin-4-amine (32-3)

To a solution of 8-(3-methoxy-2,6-dimethylphenyl)-6-(pyridin-4-yl)pyrido[3,4-d]pyrimidin-4 (3H)-one (32-2) (0.75 g, 2.09 mmol) and (2,4-dimethoxyphenyl)methanamine (0.629 mL, 4.19 mmol) in ACN (15.0 mL) were added BOP (1.203 g, 2.720 mmol), DBU (0.468 mL, 3.139 mmol) at rt. The mixture was stirred at rt for 2 h. After completion, the mixture was diluted with water (50 mL) and extracted with EtOAc (2×50 mL). The filtrate was concentrated under reduced pressure to afford N-(2,4-dimethoxybenzyl)-8-(3-methoxy-2,6-dimethylphenyl)-6-(pyridin-4-yl)pyrido[3,4-d]pyrimidin-4-amine (32-3) (1.05 g, 98%). MS (ESI) m/z: 508.69 [M+H]⁺.

### 8-(3-methoxy-2,6-dimethylphenyl)-6-(pyridin-4-yl)pyrido[3,4-d]pyrimidin-4-amine (32-4)

To a solution of N-(2,4-dimethoxybenzyl)-8-(3-methoxy-2,6-dimethylphenyl)-6-(pyridin-4-yl)pyrido[3,4-d]pyrimidin-4-amine (32-3) (1.05 g, 2.069 mmol) in DCM (10.5 mL) were added TFA (1.583 mL, 20.69 mmol) and TfOH (2.74 mL, 31.03 mmol) at rt. The mixture was stirred at rt for 2 h. After completion, the reaction was quenched with NaHCO₃ (30 mL) and extracted with DCM (2×30 mL). The filtrate was concentrated under reduced pressure. The residue was purified by column chromatography (SiO₂, EtOAc/PET) to afford 8-(3-methoxy-2,6-dimethylphenyl)-6-(pyridin-4-yl)pyrido[3,4-d]pyrimidin-4-amine (32-4) (300 mg, 40%). MS (ESI) m/z: 358.54 [M+H]⁺.

### Isolation of (S)-8-(3-methoxy-2,6-dimethylphenyl)-6-(pyridin-4-yl)pyrido[3,4-d]pyrimidin-4-amine (32-5) and (R)-8-(3-methoxy-2,6-dimethylphenyl)-6-(pyridin-4-yl)pyrido[3,4-d]pyrimidin-4-amine (32-6)

The two isomers from racemic mixture of 8-(3-methoxy-2,6-dimethylphenyl)-6-(pyridin-4-yl)pyrido[3,4-d]pyrimidin-4-amine (300 mg) were separated by chiral SFC (Lux i-Amylose-3, 30×250 mm column, 25% MeOH) to afford (S)-8-(3-methoxy-2,6-dimethylphenyl)-6-(pyridin-4-yl)pyrido[3,4-d]pyrimidin-4-amine (32-5) (105 mg, 14%), MS (ESI) m/z: 358.58 [M+H]⁺ and (R)-8-(3-methoxy-2,6-dimethylphenyl)-6-(pyridin-4-yl)pyrido[3,4-d]pyrimidin-4-amine (32-6) (110 mg, 14%), MS (ESI) m/z: 358.54 [M+H]⁺.

### (S)-3-(4-amino-6-(pyridin-4-yl)pyrido[3,4-d]pyrimidin-8-yl)-2,4-dimethylphenol (32-B)

To a stirred solution of (S)-8-(3-methoxy-2,6-dimethylphenyl)-6-(pyridin-4-yl)pyrido[3,4-d]pyrimidin-4-amine (32-5) (0.1 g, 0.280 mmol) in DCM (1.0 mL) was added BBr₃ (1M in DCM, 1.4 mL, 1.4 mmol) at 0° C. After addition, the mixture was stirred at rt for 2 h. After completion, the mixture was concentrated under reduced pressure. To the residue, ice water (20 mL) was added and stirred for 15 min. The aqueous layer was extracted with EtOAc (3×20 mL). The filtrate was concentrated under reduced pressure. The residue was purified by prep-HPLC (GEMININX C18, 10×250 mm column, ACN/ABC in water) to afford (S)-3-(4-amino-6-(pyridin-4-yl)pyrido[3,4-d]pyrimidin-8-yl)-2,4-dimethylphenol (32-B) (0.017 g, 17%). ¹H NMR (400 MHz, DMSO-d₆): δ ppm 9.14 (bs, 1H), 8.92 (s, 1H), 8.72 (d, J=6.4 Hz, 2H), 8.42-8.20 (m, 3H), 8.11-8.09 (m, 2H), 6.92 (d, J=8.0 Hz, 1H), 6.79 (d, J=8.0 Hz, 1H), 1.74 (s, 3H), 1.66 (s, 3H). MS (ESI) m/z: 344.42 [M+H]⁺.

### (R)-3-(4-amino-6-(pyridin-4-yl)pyrido[3,4-d]pyrimidin-8-yl)-2,4-dimethylphenol (32-C)

To a stirred solution of (R)-8-(3-methoxy-2,6-dimethylphenyl)-6-(pyridin-4-yl)pyrido[3,4-d]pyrimidin-4-amine (32-6) (0.1 g, 0.280 mmol) in DCM (1.0 mL) was added BBr₃ (1M in DCM, 1.40 mL, 1.4 mmol) at 0° C. and stirred at rt for 2 h. After completion, the mixture was concentrated under reduced pressure. To the residue, ice water (20 mL) was added and the mixture stirred for 15 min. The aqueous layer was extracted with EtOAc (3×20 mL). The filtrate was concentrated under reduced pressure. The residue was purified by prep-HPLC (GEMINI NX C18, 19×150 mm column, ACN/0.2% FA in water) to afford (R)-3-(4-amino-6-(pyridin-4-yl)pyrido[3,4-d]pyrimidin-8-yl)-2,4-dimethylphenol (32-C) (0.028 g, 29). ¹H NMR (400 MHz, DMSO-d₆): δ ppm 9.12 (bs, 1H), 8.92 (s, 1H), 8.72 (d, J=5.6 Hz, 2H), 8.42-8.20 (m, 3H), 8.10 (d, J=6.0 Hz, 2H), 6.92 (d, J=8.0 Hz, 1H), 6.79 (d, J=8.0 Hz, 1H), 1.74 (s, 3H), 1.66 (s, 3H). MS (ESI) m/z: 344.38 [M+H]⁺. Compounds 32-B and 32-C are atropisomers and the shown stereochemistry may be the opposite of that depicted.

### Example 33

### (S)-3-(4-amino-6-ethynylpyrido[3,4-d]pyrimidin-8-yl)-2,4-dimethylphenol (33-B) and (R)-3-(4-amino-6-ethynylpyrido[3,4-d]pyrimidin-8-yl)-2,4-dimethylphenol (33-C)

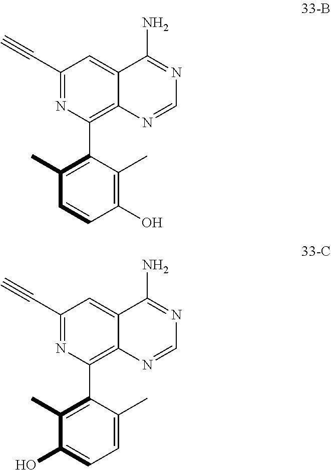

The racemic mixture of 3-(4-amino-6-ethynylpyrido[3,4-d]pyrimidin-8-yl)-2,4-dimethylphenol (33-A) was prepared following the procedure described in Example 19 with the following variations in the first step: to a solution of 6-chloro-8-(3-methoxy-2,6-dimethylphenyl)-3-((2-(trimethylsilyl) ethoxy) methyl) pyrido[3,4-d]pyrimidin-4 (3H)-one (1.0 g, 2.24 mmol), ethynyltrimethylsilane (0.661 g, 6.73 mmol) in THF (10 mL) was added triethylamine (0.92 mL, 6.73 mmol). The mixture was degassed with N₂ for 5 min followed by addition of bis(triphenylphosphine)palladium(II) dichloride (0.079 g, 0.112 mmol) and copper (I) iodide (0.043 g, 0.224 mmol). The resulting mixture was stirred at 120° C. for 16 h.

The racemic mixture of 3-(4-amino-6-ethynylpyrido[3,4-d]pyrimidin-8-yl)-2,4-dimethylphenol (33-A) (0.04 g) was subjected to chiral SFC purification (CHIRALCEL-OJ-H, 30×250 mm column, 25% MeOH) to afford the title compounds. From Peak 1: (S)-3-(4-amino-6-ethynylpyrido[3,4-d]pyrimidin-8-yl)-2,4-dimethylphenol (33-B) (5 mg, 13%). Chiral purity 99.54% (Chiralcel OJ-H, 4.6×250 mm column, 20% MeOH); ¹H NMR (400 MHz, DMSO-d₆): δ ppm 9.11 (s, 1H), 8.39 (s, 1H), 8.38 (s, 1H), 8.24 (s, 2H), 6.88 (d, J=8.4 Hz, 1H), 6.77 (d, J=8.0 Hz, 1H), 4.37 (s, 1H), 1.68 (s, 3H), 1.59 (s, 3H). MS (ESI) 291.28 [M+H]⁺. From Peak 2: (R)-3-(4-amino-6-ethynylpyrido[3,4-d]pyrimidin-8-yl)-2,4-dimethylphenol (33-C) (3 mg, 8%). Chiral purity 99.32% (Chiralcel OJ-H, 4.6×250 mm column, 20% MeOH); ¹H NMR (400 MHz, DMSO-d₆): δ ppm 9.11 (s, 1H), 8.39 (s, 1H), 8.38 (s, 1H), 8.24 (s, 2H), 6.88 (d, J=8.4 Hz, 1H), 6.76 (d, J=8.4 Hz, 1H), 4.37 (s, 1H), 1.68 (s, 3H), 1.60 (s, 3H). MS (ESI) 291.25 m/z: [M+H]⁺. Compounds 33-B and 33-C are atropisomers, and the shown stereochemistry may be the opposite of that depicted.

### Example 34

### (S)-(4-Amino-8-(3-hydroxy-2,6-dimethylphenyl)pyrido[3,4-d]pyrimidin-6-yl)(pyrrolidin-1-yl)methanone (34-B) and (R)-(4-amino-8-(3-hydroxy-2,6-dimethylphenyl)pyrido[3,4-d]pyrimidin-6-yl)(pyrrolidin-1-yl)methanone (34-C)

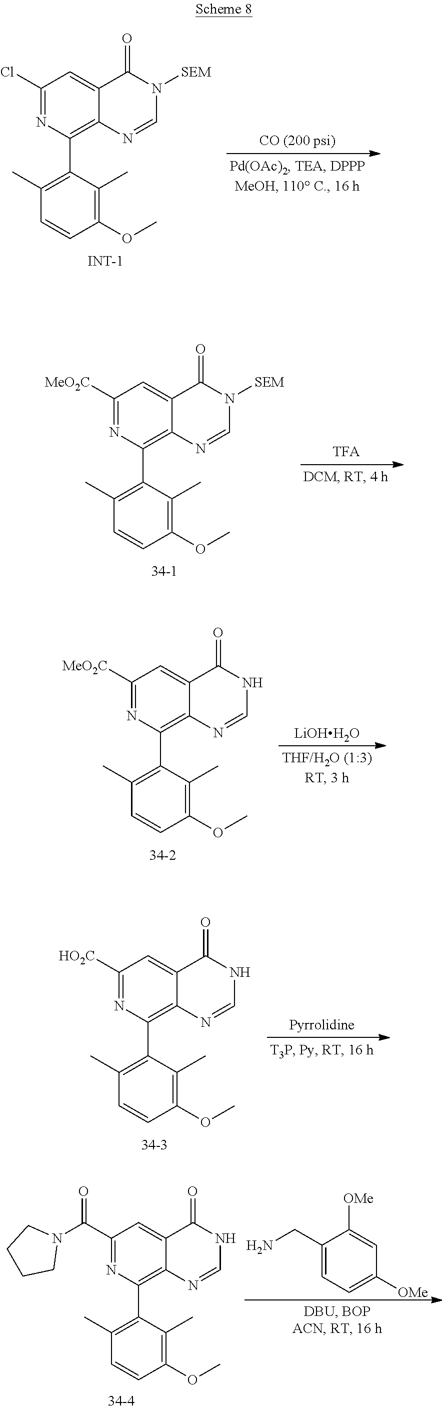

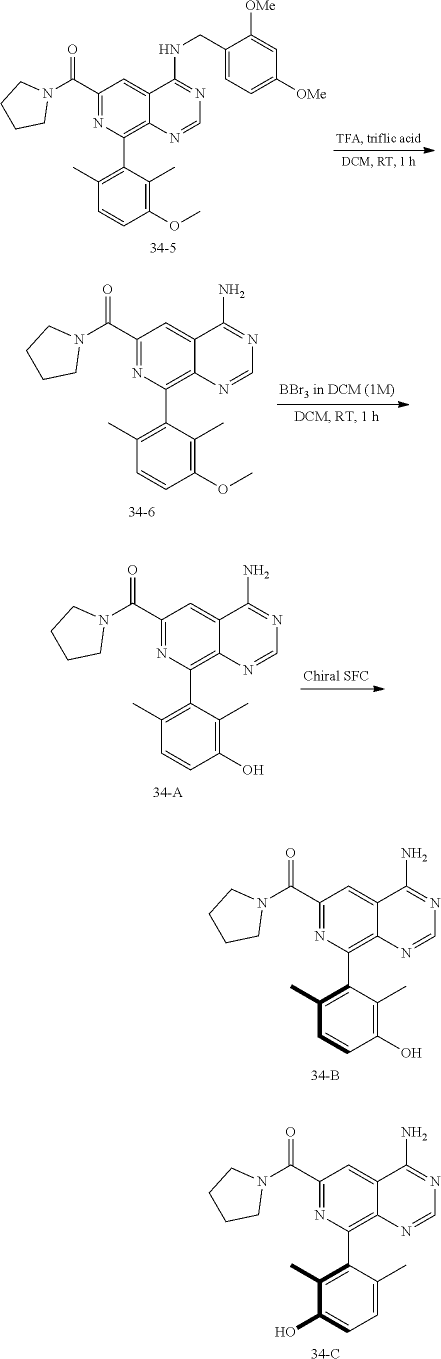

### methyl 8-(3-methoxy-2,6-dimethylphenyl)-4-oxo-3-((2-(trimethylsilyl)ethoxy)methyl)-3,4-dihydropyrido[3,4-d]pyrimidine-6-carboxylate (34-1)

To a solution of 6-chloro-8-(3-methoxy-2,6-dimethylphenyl)-3-((2-(trimethylsilyl)ethoxy)methyl)pyrido[3,4-d]pyrimidin-4 (3H)-one (INT-1) (2.0 g, 4.48 mmol) in MeOH (100 mL) was added Et₃N (3.75 mL, 26.90 mmol). The mixture was purged with N₂ for 10 min. To this mixture, DPPP (0.555 g, 1.34 mmol) and Pd(OAc)₂ (0.201 g, 0.89 mmol) were added and stirred the mixture in steel bomb under CO pressure (200 psi) at 110° C. for 16 h. The mixture was filtered through celite and washed with MeOH (80 mL). The filtrate was concentrated under reduced pressure. The residue was purified by column chromatography (SiO₂, EtOAc/PET) to afford methyl 8-(3-methoxy-2,6-dimethylphenyl)-4-oxo-3-((2-(trimethylsilyl)ethoxy)methyl)-3,4-dihydropyrido[3,4-d]pyrimidine-6-carboxylate (34-1) (1.84 g, 87% yield). MS (ESI) m/z: 470.80 [M+H]⁺.

### methyl 8-(3-methoxy-2,6-dimethylphenyl)-4-oxo-3,4-dihydropyrido[3,4-d]pyrimidine-6-carboxylate (34-2)

To a solution of methyl 8-(3-methoxy-2,6-dimethylphenyl)-4-oxo-3-((2-(trimethylsilyl)ethoxy)methyl)-3,4-dihydropyrido[3,4-d]pyrimidine-6-carboxylate (34-1) (1.4 g, 2.98 mmol) in DCM (14 mL) was added TFA (11.40 mL, 149.05 mmol) at 0° C. After addition, the mixture was stirred for 4 h. After completion, the reaction was quenched with saturated NaHCO₃ (30 mL) and extracted with EtOAc (2×30 mL). The combined organic layer was concentrated. The residue was purified by column chromatography (SiO₂, EtOAc/PET) to afford methyl 8-(3-methoxy-2,6-dimethylphenyl)-4-oxo-3,4-dihydropyrido[3,4-d]pyrimidine-6-carboxylate (34-2) (1 g, 98%). MS (ESI) m/z: 340.56 [M+H]⁺.

### 8-(3-methoxy-2,6-dimethylphenyl)-4-oxo-3,4-dihydropyrido[3,4-d]pyrimidine-6-carboxylic acid (34-3)

To a solution of methyl 8-(3-methoxy-2,6-dimethylphenyl)-4-oxo-3,4-dihydropyrido[3,4-d]pyrimidine-6-carboxylate (34-2) (0.7 g, 2.06 mmol) in a mixture of THF and H₂O (1:1, 14 mL), LiOH·H₂O (0.130 g, 3.09 mmol) was added. The mixture was stirred at rt for 3 h. After completion, the mixture was acidified with 1N HCl (~5 mL). The mixture was filtered. The product was washed with n-pentane (2×5 mL) to obtain 8-(3-methoxy-2,6-dimethylphenyl)-4-oxo-3,4-dihydropyrido[3,4-d]pyrimidine-6-carboxylic acid (34-3) (0.59 g, 87%). MS (ESI) 326.58 m/z: [M+H]⁺.

### 8-(3-methoxy-2,6-dimethylphenyl)-6-(pyrrolidine-1-carbonyl)pyrido[3,4-d]pyrimidin-4 (3H)-one (34-4)

To a stirred solution of 8-(3-methoxy-2,6-dimethylphenyl)-4-oxo-3,4-dihydropyrido[3,4-d]pyrimidine-6-carboxylic acid (34-3) (0.58 g, 1.78 mmol) in pyridine (7 mL) was added T₃P (3.18 mL, 5.34 mmol) and the mixture was stirred at rt for 5 min. To this mixture, pyrrolidine (0.74 mL, 8.90 mmol) was added. The mixture was stirred at rt for 16 h. After completion, the mixture was diluted with water (20 mL) and extracted with EtOAc (2×30 mL). The combined organic layer was washed with brine (20 mL), dried over MgSO₄, filtered, and concentrated under reduced pressure. The residue was purified by column chromatography (SiO₂, EtOAc/PET) to afford 8-(3-methoxy-2,6-dimethylphenyl)-6-(pyrrolidine-1-carbonyl)pyrido[3,4-d]pyrimidin-4 (3H)-one (34-4) (0.47 g, 69%). MS (ESI) 379.63 [M+H]⁺.

### (4-((2,4-dimethoxybenzyl)amino)-8-(3-methoxy-2,6-dimethylphenyl)pyrido[3,4-d]pyrimidin-6-yl)(pyrrolidin-1-yl)methanone (34-5)

To a solution of 8-(3-methoxy-2,6-dimethylphenyl)-6-(pyrrolidine-1-carbonyl)pyrido[3,4-d]pyrimidin-4 (3H)-one (34-4) (0.34 g, 0.89 mmol) in ACN (6.8 mL) were sequentially added DMB amine (0.150 g, 0.89 mmol), Et₃N (0.626 mL, 4.49 mmol), and BOP (0.517 g, 1.16 mmol). The mixture was stirred at rt for 16 h. After completion, the mixture was diluted with water (20 mL) and extracted with EtOAc (2×30 mL). The combined organic layer was washed with brine (20 mL), dried over MgSO₄, filtered, and concentrated. The residue was purified by column chromatography (SiO₂, EtOAc/PET) to afford (4-((2,4-dimethoxybenzyl)amino)-8-(3-methoxy-2,6-dimethylphenyl)pyrido[3,4-d]pyrimidin-6-yl)(pyrrolidin-1-yl)methanone (34-5) (0.05 g, 10%). MS (ESI) m/z: 528.58 [M+H]⁺.

### (4-amino-8-(3-methoxy-2,6-dimethylphenyl)pyrido[3,4-d]pyrimidin-6-yl)(pyrrolidin-1-yl)methanone (34-6)

To a solution of (4-((2,4-dimethoxybenzyl)amino)-8-(3-methoxy-2,6-dimethylphenyl)pyrido[3,4-d]pyrimidin-6-yl)(pyrrolidin-1-yl)methanone (34-5) (0.052 g, 0.09 mmol) in DCM (0.52 mL) were added TFA (0.015 mL, 0.19 mmol) and triflic acid (0.017 mL, 0.19 mmol) at 0° C. The mixture was stirred at rt for 1 h. After completion, the reaction was quenched with saturated NaHCO₃ (10 mL) and extracted with EtOAc (2×20 mL). The combined organic layers were concentrated. The residue was triturated with n-pentene (2×4 mL) to afford (4-amino-8-(3-methoxy-2,6-dimethylphenyl)pyrido[3,4-d]pyrimidin-6-yl)(pyrrolidin-1-yl)methanone (34-6) (0.04 g, crude). MS (ESI) m/z: 378.43 [M+H]⁺.

### (4-amino-8-(3-hydroxy-2,6-dimethylphenyl)pyrido[3,4-d]pyrimidin-6-yl)(pyrrolidin-1-yl)methanone (34-A)

To a solution of (4-amino-8-(3-methoxy-2,6-dimethylphenyl)pyrido[3,4-d]pyrimidin-6-yl)(pyrrolidin-1-yl)methanone (34-6) (0.04 g, 0.13 mmol) in DCM (0.5 mL) was added BBr₃ (1M in DCM, 0.06 mL, 0.06 mmol) at 0° C. The mixture was stirred at rt for 1 h. After completion, the reaction was quenched with saturated NaHCO₃ (10 mL) and extracted with 10% MeOH in DCM (30 mL). The organic layer was concentrated. The residue was purified by achiral SFC (DCPAK P4VP, 30×250 mm column, CO₂/30% MeOH) to afford racemic (4-amino-8-(3-hydroxy-2,6-dimethylphenyl)pyrido[3,4-d]pyrimidin-6-yl)(pyrrolidin-1-yl)methanone (34-A) (0.02 g, 41%). MS (ESI) m/z: 364.41 [M+H]⁺.

### Isolation of (S)-(4-amino-8-(3-hydroxy-2,6-dimethylphenyl)pyrido[3,4-d]pyrimidin-6-yl)(pyrrolidin-1-yl)methanone (34-B) and (R)-(4-amino-8-(3-hydroxy-2,6-dimethylphenyl)pyrido[3,4-d]pyrimidin-6-yl)(pyrrolidin-1-yl)methanone (34-C)

The racemic mixture of (4-amino-8-(3-hydroxy-2,6-dimethylphenyl)pyrido[3,4-d]pyrimidin-6-yl)(pyrrolidin-1-yl)methanone (0.02 g) was subjected to chiral SFC purification (CHIRALPAK-AS-H, 30×250 mm column, CO₂/30% ACN:IPA (1:1)) to afford the title compounds. From Peak 1: (S)-(4-amino-8-(3-hydroxy-2,6-dimethylphenyl)pyrido[3,4-d]pyrimidin-6-yl)(pyrrolidin-1-yl)methanone (34-B) (0.004 g, 8.31%). Chiral purity 99.60% (CHIRALPAK-AS-H, 30×250 mm column, CO₂/30% ACN:IPA (1:1); ¹H NMR (400 MHz, DMSO-d₆): δ ppm 9.13 (s, 1H), 8.57 (s, 1H), 8.40 (s, 1H), 8.39-8.20 (br s, 2H), 6.88 (d, J=8.40 Hz, 1H), 6.77 (d, J=8.00 Hz, 1H), 3.56-3.50 (m, 4H), 1.83-1.81 (m, 4H), 1.69 (s, 3H), 1.61 (s, 3H). MS (ESI) m/z: 364.35 [M+H]⁺. From Peak 2: (R)-(4-amino-8-(3-hydroxy-2,6-dimethylphenyl)pyrido[3,4-d]pyrimidin-6-yl)(pyrrolidin-1-yl)methanone (34-C) (0.005 g, 10.39%). Chiral purity 94.80% (CHIRALPAK-AS-H, 30×250 mm column, CO₂/30% ACN:IPA (1:1); ¹H NMR (400 MHz, DMSO-d₆): δ ppm 9.12 (s, 1H), 8.57 (s, 1H), 8.40 (s, 1H), 8.40-8.15 (br s, 2H), 6.88 (d, J=8.0 Hz, 1H), 6.77 (d, J=8.4 Hz, 1H), 3.56-3.50 (m, 4H), 1.88-1.81 (m, 4H), 1.69 (s, 3H), 1.61 (s, 3H). MS (ESI) 364.35 [M+H]⁺. Compounds 34-B and 34-C are atropisomers, and the shown stereochemistry may be the opposite of that depicted.

### Example 35

### (S)-2-(4-amino-8-(3-hydroxy-2,6-dimethylphenyl) pyrido[3,4-d]pyrimidin-6-yl)-1-morpholinoethan-1-one (35-B) and (R)-2-(4-amino-8-(3-hydroxy-2,6-dimethylphenyl) pyrido[3,4-d]pyrimidin-6-yl)-1-morpholinoethan-1-one (35-C)

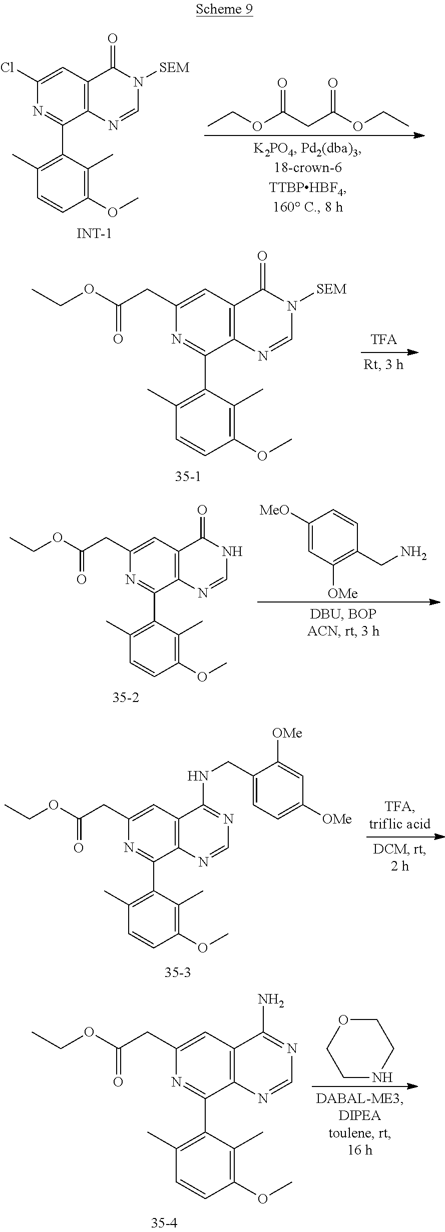

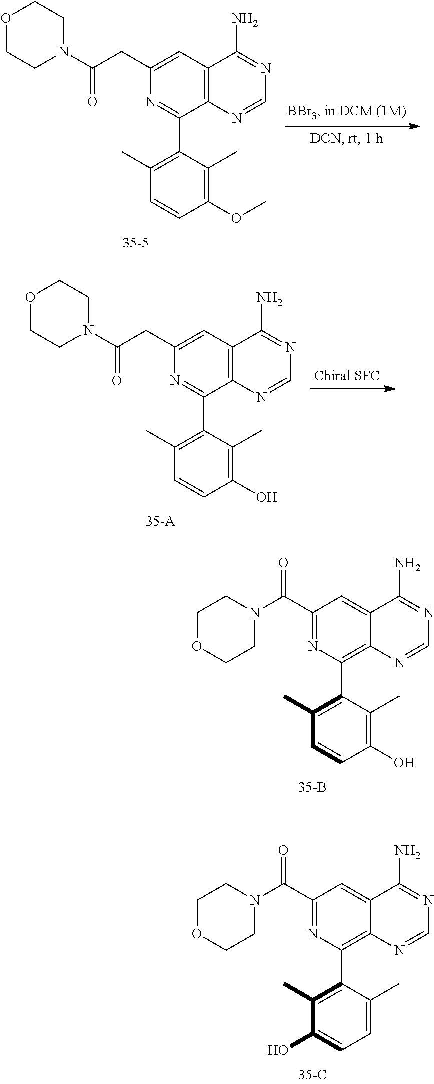

### ethyl 2-(8-(3-methoxy-2,6-dimethylphenyl)-4-oxo-3-((2-(trimethylsilyl) ethoxy) methyl)-3,4-dihydropyrido[3,4-d]pyrimidin-6-yl) acetate (35-1)

To a solution of 6-chloro-8-(3-methoxy-2,6-dimethylphenyl)-3-((2-(trimethylsilyl) ethoxy) methyl) pyrido[3,4-d]pyrimidin-4 (3H)-one (INT-1) (1.0 g, 2.25 mmol) in diethyl malonate (12 mL) were added K₃PO₄ (1.34 g, 6.29 mmol), 18-crown-6-ether (0.297 g, 1.123 mmol), and tri-tert-butylphosphonium tetra fluoroborate (0.065 g, 0.225 mmol) at rt. The mixture was degassed with N₂ for 2 min, then Pd₂(dba)₃ (0.065 g, 0.112 mmol) was added at rt. The mixture was stirred at 160° C. for 8 h. After completion, the crude was directly purified by column chromatography (SiO₂, EtOAc/PET) to afford ethyl 2-(8-(3-methoxy-2,6-dimethylphenyl)-4-oxo-3-((2-(trimethylsilyl) ethoxy) methyl)-3,4-dihydropyrido[3,4-d]pyrimidin-6-yl) acetate (35-1) (0.9 g, 80%). MS (ESI) m/z: 498.81 [M+H]⁺.

### ethyl 2-(8-(3-methoxy-2,6-dimethylphenyl)-4-oxo-3,4-dihydropyrido[3,4-d]pyrimidin-6-yl) acetate (35-2)

A solution of ethyl 2-(8-(3-methoxy-2,6-dimethylphenyl)-4-oxo-3-((2-(trimethylsilyl) ethoxy) methyl)-3,4-dihydropyrido[3,4-d]pyrimidin-6-yl) acetate (35-1) (2.0 g, 4.02 mmol) in TFA (20 mL) was stirred at rt for 2 h. After completion, the mixture was concentrated. The crude was carefully quenched with saturated NaHCO₃ (20 mL) and extracted with EtOAc (2×50 mL). The combined organic layer was washed with brine (30 mL), dried over Na₂SO₄, filtered, and concentrated to obtain ethyl 2-(8-(3-methoxy-2,6-dimethylphenyl)-4-oxo-3,4-dihydropyrido[3,4-d]pyrimidin-6-yl) acetate (35-2) (1.4 g), which was used for next step without further purification. MS (ESI) m/z: 368.38 [M+H]⁺.

### ethyl 2-(4-((2,4-dimethoxybenzyl) amino)-8-(3-methoxy-2,6-dimethylphenyl) pyrido[3,4-d]pyrimidin-6-yl) acetate (35-3)

To a solution of ethyl 2-(8-(3-methoxy-2,6-dimethylphenyl)-4-oxo-3,4-dihydropyrido[3,4-d]pyrimidin-6-yl) acetate (35-2) (1.5 g, 4.08 mmol) and (3,5-dimethoxyphenyl) methanamine (1.365 g, 8.165 mmol) in ACN (30 mL) was added TEA (1.7 mL, 12.25 mmol) and BOP (2.347 g, 5.307 mmol) at rt. The mixture was stirred at rt for 16 h. After completion, water (50 mL) was added to the mixture and extracted with EA (2×50 mL). The combined organic layer was washed with brine (30 mL), dried over Na₂SO₄, filtered, and concentrated. The residue was purified by column chromatography (SiO₂, EtOAc/PET) to afford ethyl 2-(4-((2,4-dimethoxybenzyl) amino)-8-(3-methoxy-2,6-dimethylphenyl) pyrido[3,4-d]pyrimidin-6-yl) acetate (35-3) (0.7 g, 33%). MS (ESI) m/z: 517.58 [M+H]⁺.

### ethyl 2-(4-amino-8-(3-methoxy-2,6-dimethylphenyl) pyrido[3,4-d]pyrimidin-6-yl) acetate (35-4)

To a solution of ethyl 2-(4-((2,4-dimethoxybenzyl) amino)-8-(3-methoxy-2,6-dimethylphenyl) pyrido[3,4-d]pyrimidin-6-yl) acetate (35-3) (0.550 g, 1.065 mmol) in DCM (16.5 mL) were added TFA (4.9 mL, 63.88 mmol), triflic acid (3.76 mL, 42.59 mmol) at rt. The mixture was stirred at rt for 2 h. After completion, the reaction was quenched with saturated NaHCO₃ solution. Water (50 mL) was added and extracted with EtOAc (2×50 mL). The combined organic layer was washed with brine (30 mL), dried over Na₂SO₄, filtered, and concentrated. The residue was triturated with diethyl ether to afford ethyl 2-(4-amino-8-(3-methoxy-2,6-dimethylphenyl) pyrido[3,4-d]pyrimidin-6-yl) acetate (35-4) (500 mg), which was used for next step without purification. MS (ESI) m/z: 367.66 [M+H]⁺.

### 2-(4-amino-8-(3-methoxy-2,6-dimethylphenyl) pyrido[3,4-d]pyrimidin-6-yl)-1-morpholinoethan-1-one (35-5)

To a solution of 2-(4-amino-8-(3-methoxy-2,6-dimethylphenyl) pyrido[3,4-d]pyrimidin-6-yl) acetate (35-4) (0.2 g, 0.591 mmol) in toluene (4.0 mL) were added morpholine (0.26 mL, 2.96 mmol), DABAL-Me₃ (227.3 mg, 0.887 mmol) and DIPEA (0.52 mL, 2.96 mmol) at rt. The mixture was stirred at rt for 16 h. After completion, the mixture was concentrated. Water (50 mL) was added and extracted with EA (2×50 mL). The combined organic layer was washed with brine (30 mL), dried over Na₂SO₄, filtered, and concentrated. The residue was purified by column chromatography (SiO₂, EtOAc/PET) to afford 2-(4-amino-8-(3-methoxy-2,6-dimethylphenyl) pyrido[3,4-d]pyrimidin-6-yl)-1-morpholinoethan-1-one (35-5) (0.108 g, 44%). MS (ESI) m/z: 408.74 [M+H]⁺.

### 2-(4-amino-8-(3-hydroxy-2,6-dimethylphenyl) pyrido[3,4-d]pyrimidin-6-yl)-1-morpholinoethan-1-one (35-A)

To a solution of 2-(4-amino-8-(3-methoxy-2,6-dimethylphenyl) pyrido[3,4-d]pyrimidin-6-yl)-1-morpholinoethan-1-one (35-5) (0.095 g, 0.233 mmol) in DCM (1.9 mL) was added BBr₃ (1M in DCM, 1.17 mL, 1.17 mmol) at 0° C. The mixture was stirred at rt for 1 h. After completion, the reaction was quenched with NaHCO₃ (25 mL) and extracted with 20% MeOH in DCM (3×25 mL), dried over Na₂SO₄, filtered, and concentrated. The residue was purified by preparative HPLC (Acquity UPLC BEH C18, 50×2.1 mm column, ACN/H₂O with 0.1% TFA) to obtain 2-(4-amino-8-(3-hydroxy-2,6-dimethylphenyl) pyrido[3,4-d]pyrimidin-6-yl)-1-morpholinoethan-1-one (35-A) (0.038 g). MS (ESI) m/z: 394.38 [M+H]⁺.

### Isolation of (S)-2-(4-amino-8-(3-hydroxy-2,6-dimethylphenyl) pyrido[3,4-d]pyrimidin-6-yl)-1-morpholinoethan-1-one (35-B) and (R)-2-(4-amino-8-(3-hydroxy-2,6-dimethylphenyl) pyrido[3,4-d]pyrimidin-6-yl)-1-morpholinoethan-1-one (35-C)

The racemic mixture of 2-(4-amino-8-(3-hydroxy-2,6-dimethylphenyl) pyrido[3,4-d]pyrimidin-6-yl)-1-morpholinoethan-1-one (35-A) (0.038 g) was subjected to chiral SFC purification (ethyl pyridine, 30×250 column, CO₂/20% MeOH) to afford the title compounds. From Peak 1: (S)-2-(4-amino-8-(3-hydroxy-2,6-dimethylphenyl) pyrido[3,4-d]pyrimidin-6-yl)-1-morpholinoethan-1-one (35-B) (0.014, 15.26%). Chiral purity 99.06% ethyl pyridine, 30×250 column, CO₂/20% MeOH); ¹H NMR (400 MHz, DMSO-d₆): δ ppm 9.07 (s, 1H), 8.33 (s, 1H), 8.09-8.00 (m, 2H), 7.99 (s, 1H), 6.87 (d, J=8.4 Hz, 1H), 6.75 (d, J=8.4 Hz, 1H), 4.00 (s, 2H), 3.55-3.45 (m, 8H), 1.67 (s, 3H), 1.59 (s, 3H). MS (ESI) m/z: 394.38 [M+H]⁺. From Peak 2: (R)-2-(4-amino-8-(3-hydroxy-2,6-dimethylphenyl) pyrido[3,4-d]pyrimidin-6-yl)-1-morpholinoethan-1-one (35-C) (0.014, 15.26%). Chiral purity 96.16% (ethyl pyridine, 30×250 column, CO₂/20% MeOH); ¹H NMR (400 MHz, DMSO-d₆): δ ppm 9.06 (s, 1H), 8.33 (s, 1H), 8.08-8.00 (m, 3H), 6.87 (d, J=8.4 Hz, 1H), 6.75 (d, J=8.0 Hz, 1H), 4.00 (s, 2H), 3.55-3.45 (m, 8H), 1.67 (s, 3H), 1.59 (s, 3H). MS (ESI) m/z: 394.38 [M+H]⁺. Compounds 35-7 and 35-8 are atropisomers, and the shown stereochemistry may be the opposite of that depicted.

### Example 36

### (S)-3-(4-Amino-6-(cyclopropyl(ethyl)amino)pyrido[3,4-d]pyrimidin-8-yl)-2,4-dimethylphenol (36-B) and (R)-3-(4-amino-6-(cyclopropyl(ethyl)amino)pyrido[3,4-d]pyrimidin-8-yl)-2,4-dimethylphenol (36-C)

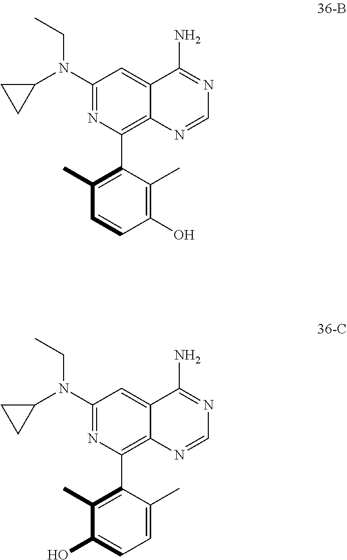

The racemic mixture of 3-(4-amino-6-(cyclopropyl(ethyl)amino)pyrido[3,4-d]pyrimidin-8-yl)-2,4-dimethylphenol (36-A) (0.09 g) was prepared following the procedure described in Example 1 and was subjected to chiral SFC purification (Chiralcel-OJ-H, 30×250 mm column, 17% ACN/ⁱPrOH (1:1) with 0.3% 7N NH₃ in MeOH) to afford the title compounds. From Peak 1: (S)-3-(4-amino-6-(cyclopropyl(ethyl)amino)pyrido[3,4-d]pyrimidin-8-yl)-2,4-dimethylphenol (36-B): (34.5 mg, 40%). Chiral purity 99.6% (Chiralcel OJ-H (4.6×250 mm column), 20% CAN/ⁱPrOH (1:1) with 0.2% DEA); ¹H NMR (400 MHz, DMSO-d₆): δ ppm 9.04 (s, 1H), 8.06 (s, 1H), 7.83 (br. S, 2H), 7.30 (s, 1H), 6.85 (d, J=8.0 Hz, 1H), 6.73 (d, J=8.0 Hz, 1H), 3.71-3.61 (m, 2H), 2.56-2.52 (m, 1H), 1.72 (s, 3H), 1.63 (s, 3H), 1.07-0.97 (m, 5H), 0.65-0.59 (m, 2H). MS (ESI) m/z: 350.4 [M+H]⁺. From Peak 2: (R)-3-(4-amino-6-(cyclopropyl(ethyl)amino)pyrido[3,4-d]pyrimidin-8-yl)-2,4-dimethylphenol (36-C) (29.7 mg, 34%). Chiral purity 93.2% (Chiralcel OJ-H (4.6×250 mm column), 20% ACN/ⁱPrOH (1:1) with 0.2% DEA); ¹H NMR (400 MHz, DMSO-d₆): δ ppm 9.05 (s, 1H), 8.07 (s, 1H), 7.84 (br. s, 2H), 7.31 (s, 1H), 6.86 (d, J=8.4 Hz, 1H), 6.74 (d, J=8.0 Hz, 1H), 3.71-3.60 (m, 2H), 2.56-2.52 (m, 1H), 1.72 (s, 3H), 1.63 (s, 3H), 1.08-0.98 (m, 5H), 0.66-0.59 (m, 2H). MS (ESI) m/z: 350.4 [M+H]⁺. Compounds 36-B and 36-C are atropisomers, and the shown stereochemistry may be the opposite of that depicted.

### Example 37

### (S)-3-(4-Amino-6-(cyclobutyl(methyl)amino)pyrido[3,4-d]pyrimidin-8-yl)-2,4-dimethylphenol (37-B) and (R)-3-(4-amino-6-(cyclobutyl(methyl)amino)pyrido[3,4-d]pyrimidin-8-yl)-2,4-dimethylphenol (37-C)

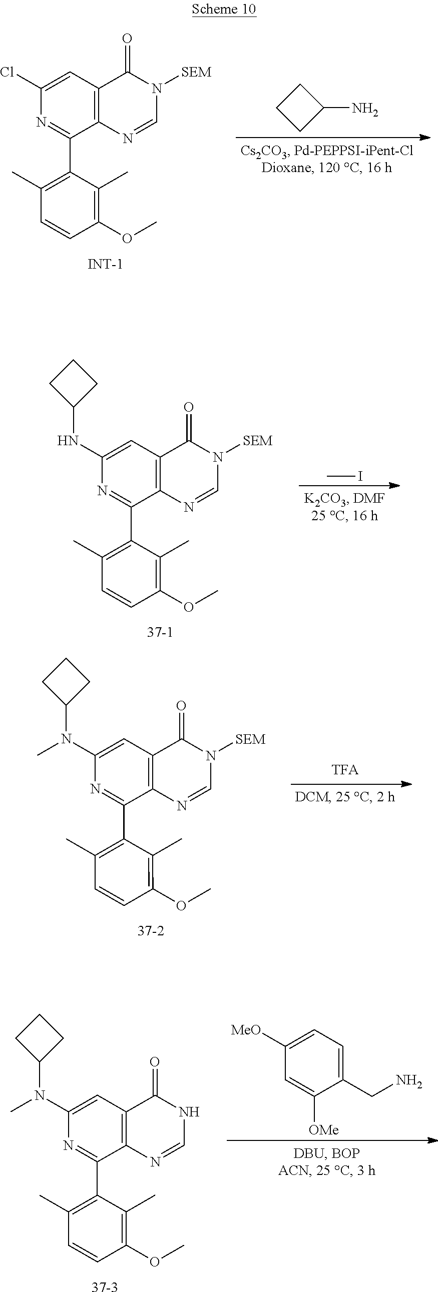

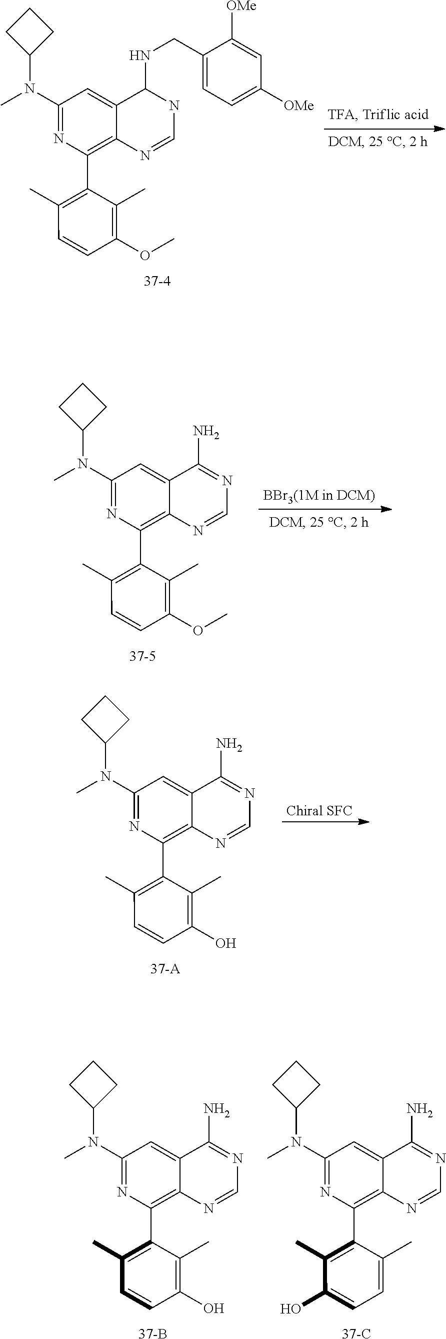

### 6-(cyclobutylamino)-8-(3-methoxy-2,6-dimethylphenyl)-3-((2-(trimethylsilyl)ethoxy)methyl)pyrido[3,4-d]pyrimidin-4 (3H)-one (37-1)

To a solution of 6-chloro-8-(3-methoxy-2,6-dimethylphenyl)-3-((2-(trimethylsilyl)ethoxy)methyl)pyrido[3,4-d]pyrimidin-4 (3H)-one (INT-1) (0.70 g, 1.57 mmol) and cyclobutanamine (0.56 g, 7.85 mmol) in 1,4-dioxane (21 mL) was added Cs₂CO₃ (1.53 g, 4.71 mmol) and degassed with N₂ for 5 min. followed by addition of Pd-PEPPSI-iPentCl (0.12 g, 0.16 mmol). The mixture was degassed again for 5 min. and stirred at 120° C. for 16 h. After completion, the reaction was quenched with water (100 mL) and extracted with EtOAc (2×100 mL). The combined organic layers were washed with brine (20 mL), dried over MgSO₄, filtered, and concentrated. The residue was purified by column chromatography (SiO₂, EtOAc/PET) to afford 6-(cyclobutylamino)-8-(3-methoxy-2,6-dimethylphenyl)-3-((2-(trimethylsilyl)ethoxy)methyl)pyrido[3,4-d]pyrimidin-4 (3H)-one (37-1) (0.70 g, 92%). MS (ESI) m/z: 481.8 [M+H]⁺.

### 6-(cyclobutyl(methyl)amino)-8-(3-methoxy-2,6-dimethylphenyl)-3-((2-(trimethylsilyl)ethoxy)methyl)pyrido[3,4-d]pyrimidin-4 (3H)-one (37-2)

To a solution of 6-(cyclobutylamino)-8-(3-methoxy-2,6-dimethylphenyl)-3-((2-(trimethylsilyl)ethoxy)methyl)pyrido[3,4-d]pyrimidin-4 (3H)-one (37-1) (0.70 g, 1.46 mmol) in DMF (21 mL) was added NaH (60% in mineral oil, 0.18 g, 4.37 mmol) at 0° C. The mixture was stirred at rt for 30 min. followed by addition of CH₃I (0.27 mL, 0.37 mmol). The mixture was stirred at rt for 16 h. After completion, the reaction mixture was quenched with ice cold water (50 mL) and extracted with EtOAc (2×50 mL). The combined organic layers were washed with brine (20 mL), dried over MgSO₄, filtered, and concentrated. The material was taken to the next step without purification (37-2) (0.67 g, quantitative). MS (ESI) m/z: 495.9 [M+H]⁺.

### 6-(cyclobutyl(methyl)amino)-8-(3-methoxy-2,6-dimethylphenyl)pyrido[3,4-d]pyrimidin-4 (3H)-one (37-3)

To a solution of 6-(cyclobutyl(methyl)amino)-8-(3-methoxy-2,6-dimethylphenyl)-3-((2-(trimethylsilyl)ethoxy)methyl)pyrido[3,4-d]pyrimidin-4 (3H)-one (37-2) (0.65 g, 1.31 mmol) in DCM (6.5 mL) were added TFA (3.25 mL) at 25° C., and the mixture was stirred for 16 h. After completion, the reaction was quenched with sat. NaHCO₃ (50 mL) and extracted with EtOAc (3×50 mL). The combined organic layers were washed with brine (50 mL), dried over MgSO₄, filtered, and concentrated to afford 6-(cyclobutyl(methyl)amino)-8-(3-methoxy-2,6-dimethylphenyl)pyrido[3,4-d]pyrimidin-4 (3H)-one (37-3) (0.43 g, 89%). The material was taken to the next step without further purification. MS (ESI) m/z: 365.7 [M+H]⁺.

### N⁶-cyclobutyl-N⁴-(2,4-dimethoxybenzyl)-8-(3-methoxy-2,6-dimethylphenyl)-N⁶-methylpyrido[3,4-d]pyrimidine-4,6-diamine (37-4)

To a solution of 6-(cyclobutyl(methyl)amino)-8-(3-methoxy-2,6-dimethylphenyl)pyrido[3,4-d]pyrimidin-4 (3H)-one (37-3) (0.42 g, 1.15 mmol) in ACN (4.2 mL) were added (2,4-dimethoxyphenyl)methanamine (0.39 g, 2.31 mmol), DBU (0.35 g, 2.31 mmol), and BOP (0.77 g, 1.73 mmol) at 0° C. The mixture was stirred at 25° C. for 16 h. After completion, the reaction was quenched with water (60 mL) and extracted with EtOAc (2×60 mL). The combined organic layers were washed with brine (60 mL), dried over MgSO₄, filtered, and concentrated. The residue was purified by column chromatography (SiO₂, MeOH/CH₂Cl₂) to afford N⁶-cyclobutyl-N⁴-(2,4-dimethoxybenzyl)-8-(3-methoxy-2,6-dimethylphenyl)-N⁶-methylpyrido[3,4-d]pyrimidine-4,6-diamine (37-4) (0.36 g, 60%). MS (ESI) m/z: 514.6 [M+H]⁺.

### N⁶-cyclobutyl-8-(3-methoxy-2,6-dimethylphenyl)-N⁶-methylpyrido[3,4-d]pyrimidine-4,6-diamine (37-5)

To a solution of N⁶-cyclobutyl-N⁴-(2,4-dimethoxybenzyl)-8-(3-methoxy-2,6-dimethylphenyl)-N⁶-methylpyrido[3,4-d]pyrimidine-4,6-diamine (37-4) (0.65 g, 1.27 mmol) in DCM (6.5 mL) were added TFA (3.25 mL) and TfOH (0.65 mL) at 0° C. The mixture was stirred at 25° C. for 2 h. After completion, the reaction was quenched with sat. NaHCO₃ (60 mL) and extracted with EtOAc (2×60 mL). The combined organic layers were washed with brine (50 mL), dried over MgSO₄, filtered, and concentrated to afford N⁶-cyclobutyl-8-(3-methoxy-2,6-dimethylphenyl)-M-methylpyrido[3,4-d]pyrimidine-4,6-diamine. The material was taken to the next step without further purification (37-5) (0.32 g, quantitative). MS (ESI) m/z: 364.7 [M+H]⁺.

### 3-(4-amino-6-(cyclobutyl(methyl)amino)pyrido[3,4-d]pyrimidin-8-yl)-2,4-dimethylphenol (37-A)

To a stirred solution of N⁶-cyclobutyl-8-(3-methoxy-2,6-dimethylphenyl)-N⁶-methylpyrido[3,4-d]pyrimidine-4,6-diamine (37-5) (0.30 g, 0.83 mmol) in DCM (3 mL) was added BBr₃ (1M in DCM, 2.48 mL, 2.48 mmol) at 0° C. The mixture was stirred at 25° C. for 2 h. After completion, the reaction was quenched with sat. NaHCO₃ (50 mL) and extracted with EtOAc (2×50 mL). The combined organic layers were washed with brine (50 mL), dried over MgSO₄, filtered, and concentrated. The residue was purified by column chromatography (SiO₂, MeOH/CH₂Cl₂) to afford a racemic 3-(4-amino-6-(cyclobutyl(methyl)amino)pyrido[3,4-d]pyrimidin-8-yl)-2,4-dimethylphenol (37-A) (0.19 g, 65%). MS (ESI) m/z: 350.6 [M+H]⁺.

### Isolation of (S)-3-(4-amino-6-(cyclobutyl(methyl)amino)pyrido[3,4-d]pyrimidin-8-yl)-2,4-dimethylphenol (37-B) and (R)-3-(4-amino-6-(cyclobutyl(methyl)amino)pyrido[3,4-d]pyrimidin-8-yl)-2,4-dimethylphenol (37-C)

The racemic mixture of 3-(4-amino-6-(cyclobutyl(methyl)amino)pyrido[3,4-d]pyrimidin-8-yl)-2,4-dimethylphenol (37-A) was subjected to chiral SFC purification (Chiralpak-IC, 30×250 mm column, 27% MeOH) to afford the title compounds. From Peak 1: (S)-3-(4-amino-6-(cyclobutyl(methyl)amino)pyrido[3,4-d]pyrimidin-8-yl)-2,4-dimethylphenol (37-B) (70 mg, 38%). Chiral purity 99.7% (Chiralpak IC (4.6×250 mm column), 30% IPA with 0.2% ⁱPrNH₂); ¹H NMR (400 MHz, DMSO-d₆): δ ppm 9.03 (s, 1H), 8.03 (s, 1H), 7.75 (br. s, 2H), 7.01 (s, 1H), 6.85 (d, J=8 Hz, 1H), 6.73 (d, J=8 Hz, 1H), 4.93-4.84 (m, 1H), 3.01 (s, 3H), 2.23-2.10 (m, 4H), 1.72 (s, 3H), 1.69-1.54 (m, 5H). MS (ESI) m/z: 350.4 [M+H]⁺. From Peak 2: (R)-3-(4-amino-6-(cyclobutyl(methyl)amino)pyrido[3,4-d]pyrimidin-8-yl)-2,4-dimethylphenol (37-C) (45 mg, 25%). Chiral purity 99.9% (Chiralpak IC (4.6×250 mm column), 30% IPA with 0.2% PrNH₂); ¹H NMR (400 MHz, DMSO-d₆): δ ppm 9.03 (s, 1H), 8.03 (s, 1H), 7.75 (br. s, 2H), 7.01 (s, 1H), 6.85 (d, J=8 Hz, 1H), 6.73 (d, J=8 Hz, 1H), 4.96-4.83 (m, 1H), 3.01 (s, 3H), 2.28-2.11 (m, 4H), 1.72 (s, 3H), 1.69-1.56 (m, 5H). MS (ESI) m/z: 350.6 [M+H]⁺. Compounds 37-B and 37-C are atropisomers, and the shown stereochemistry may be the opposite of that depicted.

### Example 38

### 3-((S)-4-Amino-6-((S)-1-oxa-6-azaspiro[3.4]octan-6-yl)pyrido[3,4-d]pyrimidin-8-yl)-2,4-dimethylphenol (38-B), 3-((R)-4-amino-6-((S)-1-oxa-6-azaspiro[3.4]octan-6-yl)pyrido[3,4-d]pyrimidin-8-yl)-2,4-dimethylphenol (38-C), 3-((S)-4-amino-6-((R)-1-oxa-6-azaspiro[3.4]octan-6-yl)pyrido[3,4-d]pyrimidin-8-yl)-2,4-dimethylphenol (38-D) and 3-((R)-4-amino-6-((R)-1-oxa-6-azaspiro[3.4]octan-6-yl) pyrido[3,4-d]pyrimidin-8-yl)-2,4-dimethylphenol (38-E)

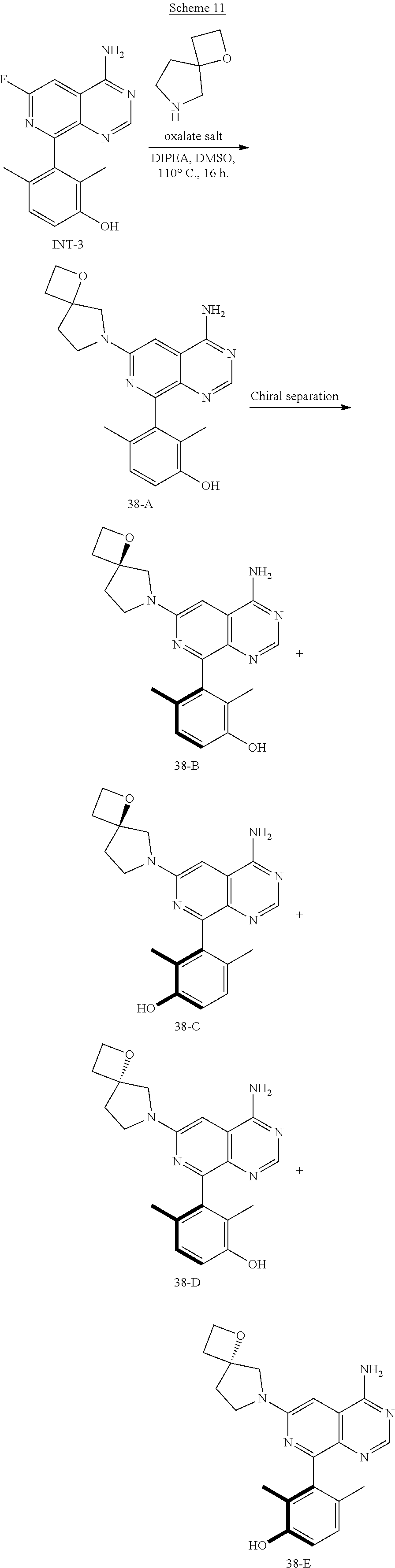

### 3-(4-amino-6-(1-oxa-6-azaspiro[3.4]octan-6-yl)pyrido[3,4-d]pyrimidin-8-yl)-2,4-dimethylphenol (38-1)

To a solution 3-(4-amino-6-fluoropyrido[3,4-d]pyrimidin-8-yl)-2,4-dimethylphenol (INT-3) (0.20 g, 0.70 mmol) in DMSO (10 mL) were added 1-oxa-6-azaspiro[3.4]octane (0.89 g, 2.81 mmol) and DIPEA (1.22 mL, 7.04 mmol) at rt. The mixture was heated at 110° C. for 16 h. After completion, the mixture was diluted with ice cold water (50 mL) and extracted with EtOAc (2×80 mL). The combined organic layers were washed with brine (60 mL), dried over Na₂SO₄, filtered, and concentrated. The residue was purified by prep-HPLC (X Bridge Phenyl C18, 19×250 mm column, ACN/H₂O/10 mM NH₄HCO₃) to afford a diastereomeric 3-(4-amino-6-(1-oxa-6-azaspiro[3.4]octan-6-yl)pyrido[3,4-d]pyrimidin-8-yl)-2,4-dimethylphenol (38-A) (0.036 g, 13%). MS (ESI) m/z: 378.6 [M+H]⁺.

### Isolation of 3-((S)-4-amino-6-((S)-1-oxa-6-azaspiro[3.4]octan-6-yl)pyrido[3,4-d]pyrimidin-8-yl)-2,4-dimethylphenol (38-B), 3-((R)-4-amino-6-((S)-1-oxa-6-azaspiro[3.4]octan-6-yl)pyrido[3,4-d]pyrimidin-8-yl)-2,4-dimethylphenol (38-C), 3-((S)-4-amino-6-((R)-1-oxa-6-azaspiro[3.4]octan-6-yl)pyrido[3,4-d]pyrimidin-8-yl)-2,4-dimethylphenol (38-D) and 3-((R)-4-amino-6-((R)-1-oxa-6-azaspiro[3.4]octan-6-yl)pyrido[3,4-d]pyrimidin-8-yl)-2,4-dimethylphenol (38-E)

The diastereomeric mixture of 3-(4-amino-6-(1-oxa-6-azaspiro[3.4]octan-6-yl)pyrido[3,4-d]pyrimidin-8-yl)-2,4-dimethylphenol (36 mg) was subjected to chiral SFC purification (Chiralcel OJ-H, 30×250 mm column, 20% ACN/MeOH (1:1) with 0.2% 7N NH₃ in MeOH) to afford the title compounds. From Peak 1: 3-((S)-4-amino-6-((S)-1-oxa-6-azaspiro[3.4]octan-6-yl)pyrido[3,4-d]pyrimidin-8-yl)-2,4-dimethylphenol (38-B): (5.24 mg, 15%). Chiral purity 99.9% (Chiralcel (4.6×150 mm column), 20% ACN/MeOH (1:1) with 0.2% 7N NH₃ in MeOH); ¹H NMR (400 MHz, DMSO-d₆): δ ppm 8.99 (br. s, 1H), 8.02 (s, 1H), 7.72 (br. s, 2H), 6.90 (s, 1H), 6.85 (d, J=8.0 Hz, 1H), 6.73 (d, J=8.0 Hz, 1H), 4.48-4.38 (m, 2H), 3.80 (d, J=11.6 Hz, 1H), 3.58-3.49 (m, 2H), 3.45-3.35 (m, 1H), 2.80-2.63 (m, 2H), 2.43-2.35 (m, 1H), 2.23-2.12 (m, 1H), 1.73 (s, 3H), 1.63 (s, 3H). MS (ESI) m/z: 378.3 [M+H]⁺. From Peak 2: 3-((R)-4-amino-6-((S)-1-oxa-6-azaspiro[3.4]octan-6-yl)pyrido[3,4-d]pyrimidin-8-yl)-2,4-dimethylphenol (38-C) (6.28 mg, 17%). Chiral purity 94.8% (Chiralcel OJ-H (4.6×150 mm column), 20% ACN/MeOH (1:1) with 0.2% 7N NH₃ in MeOH); ¹H NMR (400 MHz, DMSO-d₆): δ ppm 9.30-8.20 (m, 1H), 8.02 (s, 1H), 7.72 (br. s, 2H), 6.90 (s, 1H), 6.85 (d, J=8.0 Hz, 1H), 6.73 (d, J=8.0 Hz, 1H), 4.49-4.38 (m, 2H), 3.80 (d, J=11.6 Hz, 1H), 3.58-3.50 (m, 2H), 3.43-3.35 (m, 1H), 2.80-2.65 (m, 2H), 2.42-2.35 (m, 1H), 2.25-2.13 (m, 1H), 1.72 (s, 3H), 1.64 (s, 3H). MS (ESI) m/z: 378.3 [M+H]⁺. From Peak 3: 3-((S)-4-amino-6-((R)-1-oxa-6-azaspiro[3.4]octan-6-yl)pyrido[3,4-d]pyrimidin-8-yl)-2,4-dimethylphenol (38-D) (6.35 mg, 18%). Chiral purity 96.6% (Chiralcel OJ-H (4.6×150 mm column), 20% ACN/MeOH (1:1) with 0.2% 7N NH₃ in MeOH); ¹H NMR (400 MHz, DMSO-d₆): δ ppm 8.90 (br. s, 1H), 8.02 (s, 1H), 7.72 (br. s, 2H), 6.90 (s, 1H), 6.85 (d, J=8.0 Hz, 1H), 6.73 (d, J=8.0 Hz, 1H), 4.48-4.38 (m, 2H), 3.80 (d, J=12.0 Hz, 1H), 3.59-3.50 (m, 2H), 3.45-3.35 (m, 1H), 2.80-2.63 (m, 2H), 2.44-2.35 (m, 1H), 2.25-2.15 (m, 1H), 1.72 (s, 3H), 1.64 (s, 3H). MS (ESI) m/z: 378.4 [M+H]⁺. From Peak 4: 3-((R)-4-amino-6-((R)-1-oxa-6-azaspiro[3.4]octan-6-yl)pyrido[3,4-d]pyrimidin-8-yl)-2,4-dimethylphenol (38-E) (6.66 mg, 19%). Chiral purity 97.6% (Chiralcel OJ-H (4.6×150 mm column), 20% ACN/MeOH (1:1) with 0.2% 7N NH₃ in MeOH); ¹H NMR (400 MHz, DMSO-d₆): δ ppm 8.68 (br. s, 1H), 8.02 (s, 1H), 7.72 (br. s, 2H), 6.90 (s, 1H), 6.85 (d, J=8.0 Hz, 1H), 6.73 (d, J=8.4 Hz, 1H), 4.49-4.38 (m, 2H), 3.80 (d, J=11.6 Hz, 1H), 3.58-3.50 (m, 2H), 3.45-3.35 (m, 1H), 2.80-2.65 (m, 2H), 2.45-2.35 (m, 1H), 2.25-2.15 (m, 1H), 1.73 (s, 3H), 1.63 (s, 3H). MS (ESI) m/z: 378.3 [M+H]⁺. Compounds 38-B, 38-C, 38-D, and 38-E are diastereoisomers, and the shown stereochemistry was arbitrarily assigned.

### Example 39

### (S)-3-(4-amino-6-(trifluoromethyl) pyrido[3,4-d]pyrimidin-8-yl)-2,4-dimethylphenol (39-B) and (R)-3-(4-amino-6-(trifluoromethyl) pyrido[3,4-d]pyrimidin-8-yl)-2,4-dimethylphenol (39-C)

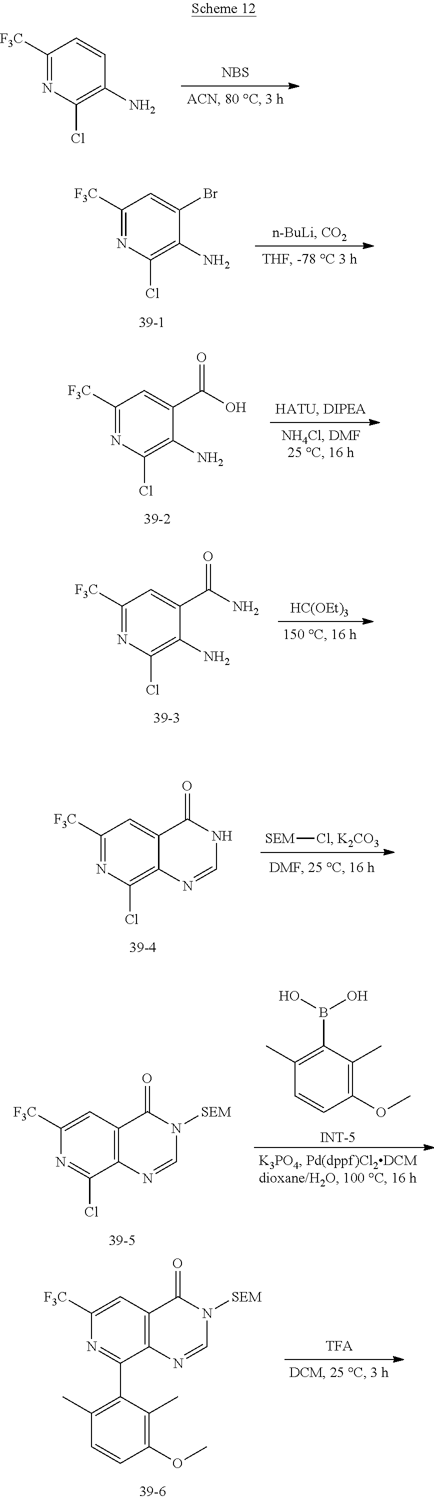

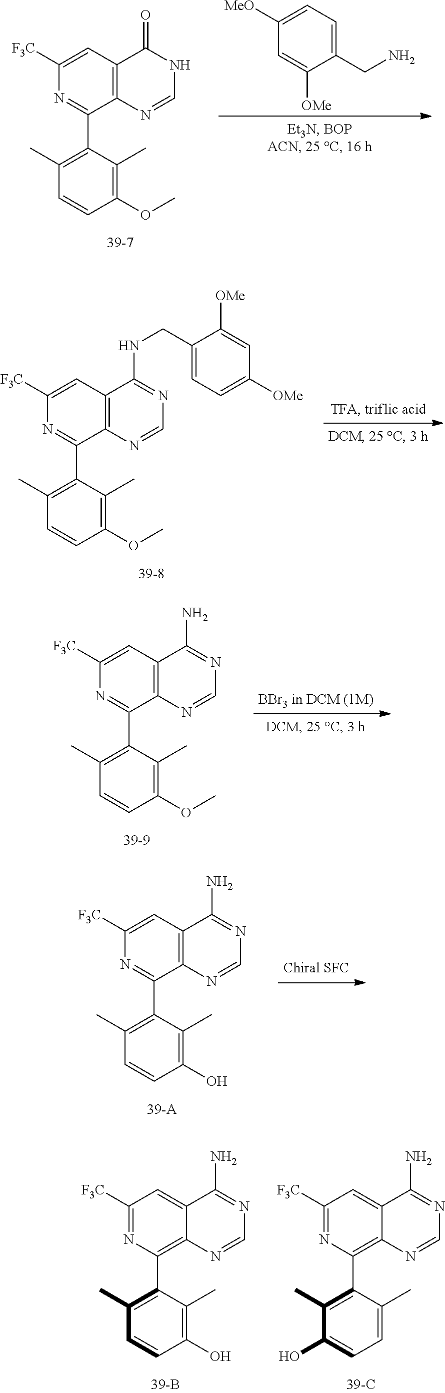

### 4-bromo-2-chloro-6-(trifluoromethyl) pyridin-3-amine (39-1)

To a solution of 2-chloro-6-(trifluoromethyl) pyridin-3-amine (20.0 g, 102 mmol) in ACN (200 mL) was added NBS (19.922 g, 111.93 mmol) at rt. The mixture was stirred 80° C. for 3 h. After completion, the mixture was concentrated. The residue was purified by column chromatography (SiO₂, EtOAc/PET) to afford 4-bromo-2-chloro-6-(trifluoromethyl)pyridin-3-amine (39-1) (22 g, 78%). MS (ESI) m/z: 277.29 [M+H]⁺.

### 3-amino-2-chloro-6-(trifluoromethyl)isonicotinic acid (39-2)

To a solution of 4-bromo-2-chloro-6-(trifluoromethyl) pyridin-3-amine (39-1) (10.0 g, 36.3 mmol) in THF (100 mL) was added n-BuLi (2 M in cyclohexane, 72.62 mL, 145.23 mmol) at −78° C. and the mixture was stirred for 30 min. Dry CO₂ gas was purged into the mixture and the mixture was stirred at −78° C. for additional 3 h. After completion, the reaction was quenched with saturated NH₄Cl solution (100 mL) and extracted with Et₂O (3×100 mL). A 2N HCl was added to adjust pH~5. Aqueous layer was extracted with EtOAc (3×250 mL). The combined organic layer was washed with water (100 mL), dried over MgSO₄, filtered, and concentrated to obtain 3-amino-2-chloro-6-(trifluoromethyl) isonicotinic acid (39-2) (1.0 g, 11%). The crude was used to the next step without further purification. MS (ESI) m/z: 241.30 [M+H]⁺.

### 3-amino-2-chloro-6-(trifluoromethyl)isonicotinamide (39-3)

To a solution of 3-amino-2-chloro-6-(trifluoromethyl)isonicotinic acid (39-2) (1.0 g, 4.16 mmol) in DMF (10 mL) were added HATU (1.739 g, 4.573 mmol) and NH₄Cl (2.224 g, 41.57 mmol). The mixture was stirred at rt for 10 min. DIPEA (7.46 mL, 41.6 mmol) was added and the mixture was stirred at rt for 16 h. After completion, water (100 mL) was added and extracted with EtOAc (2×150 mL). The combined organic layer was washed with brine (100 mL), dried over MgSO₄, filtered, and concentrated. The residue was purified by column chromatography (SiO₂, EtOAc/PET) to afford 3-amino-2-chloro-6-(trifluoromethyl) isonicotinamide (39-3) (0.6 g, 60%). MS (ESI) m/z: 240.31 [M+H]⁺.

### 8-chloro-6-(trifluoromethyl) pyrido[3,4-d]pyrimidin-4 (3H)-one (39-4)

A solution of 3-amino-2-chloro-6-(trifluoromethyl) isonicotinamide (39-3) (0.5 g, 2.09 mmol) in triethoxymethane (20 mL) was stirred at 150° C. for 16 h. After completion, the mixture was cooled to rt and concentrated to obtain 8-chloro-6-(trifluoromethyl) pyrido[3,4-d]pyrimidin-4 (3H)-one (39-4) (0.5 g). The crude was used to the next step without further purification. MS (ESI) m/z: 250.35 [M+H]⁺.

### 8-chloro-6-(trifluoromethyl)-3-((2-(trimethylsilyl) ethoxy) methyl) pyrido[3,4-d]pyrimidin-4 (3H)-one (39-5)

To a solution of 8-chloro-6-(trifluoromethyl) pyrido[3,4-d]pyrimidin-4 (3H)-one (39-4) (0.5 g, 2.003 mmol) in DMF (5 mL) was added K₂CO₃ (0.831 g, 6.01 mmol) and the mixture was stirred at rt for 30 min. The mixture was cooled to 0° C. and SEM-Cl (1.06 mL, 6.01 mmol) was added. The mixture was stirred at rt for 16 h. After completion, water (100 mL) was added and the mixture was extracted with EtOAc (2×150 mL). The combined organic layer was washed with brine (100 mL), dried over MgSO₄, filtered, and concentrated. The residue was purified by column chromatography (SiO₂, EtOAc/PET) to afford 8-chloro-6-(trifluoromethyl)-3-((2-(trimethylsilyl) ethoxy) methyl) pyrido[3,4-d]pyrimidin-4 (3H)-one (39-5) (0.5 g, 65%). ¹H NMR (400 MHz, CDCl₃): δ ppm 8.41 (s, 1H), 7.78 (d, J=8.40 Hz, 1H), 5.46 (s, 2H), 3.66-3.67 (m, 2H), 0.93-0.95 (m, 2H), 0.02 (s, 9H).

### 8-(3-methoxy-2,6-dimethylphenyl)-6-(trifluoromethyl)-3-((2-(trimethylsilyl) ethoxy) methyl) pyrido[3,4-d]pyrimidin-4 (3H)-one (39-6)

To a solution of 8-chloro-6-(trifluoromethyl)-3-((2-(trimethylsilyl) ethoxy) methyl) pyrido[3,4-d]pyrimidin-4 (3H)-one (39-5) (0.5 g, 1.32 mmol) in a mixture of 1,4 dioxane (4.0 mL) and water (1.0 mL) were added (3-methoxy-2,6-dimethylphenyl) boronic acid (0.474 g, 2.63 mmol), K₃PO₄ (0.838 g, 3.95 mmol), and Pd(dppf)Cl₂·DCM (0.161 g, 0.197 mmol) at rt. The mixture was stirred at 100° C. for 16 h. After completion, water (50 mL) was added and extracted with EtOAc (2×100 mL). The combined organic layer was washed with brine (50 mL), dried over MgSO₄, filtered, and concentrated under reduced pressure. The residue was purified by column chromatography (SiO₂, EtOAc/PET) to afford 8-(3-methoxy-2,6-dimethylphenyl)-6-(trifluoromethyl)-3-((2-(trimethylsilyl) ethoxy) methyl) pyrido[3,4-d]pyrimidin-4 (3H)-one (39-6) (0.5 g, 79%). MS (ESI) m/z: 480.48 [M+H]⁺.

### 8-(3-methoxy-2,6-dimethylphenyl)-6-(trifluoromethyl) pyrido[3,4-d]pyrimidin-4 (3H)-one (39-7)

To a solution of 8-(3-methoxy-2,6-dimethylphenyl)-6-(trifluoromethyl)-3-((2-(trimethylsilyl) ethoxy) methyl) pyrido[3,4-d]pyrimidin-4 (3H)-one (39-6) (0.5 g, 1.04 mmol) in DCM (5 ml) was added TFA (5 mL) at rt. The mixture was stirred at rt for 3 h. After completion, the reaction was quenched by NaHCO₃ solution (100 mL) and extracted with EtOAc (2×100 mL). The combined organic layer was washed with brine (20 mL), dried over MgSO₄, filtered, and concentrated to obtain 8-(3-methoxy-2,6-dimethylphenyl)-6-(trifluoromethyl) pyrido[3,4-d]pyrimidin-4 (3H)-one (39-7) (0.3 g, 82%). The crude was used to next step without further purification. MS (ESI) m/z: 350.60 [M+H]⁺.

### N-(2,4-dimethoxybenzyl)-8-(3-methoxy-2,6-dimethylphenyl)-6-(trifluoromethyl) pyrido[3,4-d]pyrimidin-4-amine (39-8)

To a solution of 8-(3-methoxy-2,6-dimethylphenyl)-6-(trifluoromethyl)pyrido[3,4-d]pyrimidin-4 (3H)-one (39-7) (0.30 g, 0.86 mmol) in ACN (5 mL) were added (2,4-dimethoxyphenyl)methanamine (0.479 g, 2.86 mmol), BOP (0.760 g, 1.718 mmol) and TEA (0.402 mL, 2.863 mmol) at rt. The mixture was stirred at rt for 16 h. After completion, water (50 mL) was added and the mixture was extracted with EtOAc (2×100 mL). The combined organic layer was washed with brine (50 mL), dried over MgSO₄, filtered, and concentrated. The residue was purified by column chromatography (SiO₂, EtOAc/PET) to afford N-(2,4-dimethoxybenzyl)-8-(3-methoxy-2,6-dimethylphenyl)-6-(trifluoromethyl)pyrido[3,4-d]pyrimidin-4-amine (39-8) (0.250 g, 58%). MS (ESI) m/z: 499.80 [M+H]⁺.

### 8-(3-methoxy-2,6-dimethylphenyl)-6-(trifluoromethyl) pyrido[3,4-d]pyrimidin-4-amine (39-9)

To a solution of N-(2,4-dimethoxybenzyl)-8-(3-methoxy-2,6-dimethylphenyl)-6-(trifluoromethyl)pyrido[3,4-d]pyrimidin-4-amine (39-8)(0.240 g, 0.481 mmol) in DCM (4.8 mL) were added TFA (4.8 mL) and triflic acid (2.4 mL) at 0° C. The mixture was stirred at rt for 3 h. After completion, the reaction was quenched with saturated NaHCO₃ (50 mL) and extracted with EtOAc (2×100 mL). The combined organic layer was washed with brine (50 mL), dried over MgSO₄, filtered, and concentrated to obtain 8-(3-methoxy-2,6-dimethylphenyl)-6-(trifluoromethyl)pyrido[3,4-d]pyrimidin-4-amine (39-9) (0.120 g, 71%). The crude was used to the next step without further purification. MS (ESI) m/z: 349.33 [M+H]⁺.

### 3-(4-amino-6-(trifluoromethyl) pyrido[3,4-d]pyrimidin-8-yl)-2,4-dimethylphenol (39-A)

To a solution of 8-(3-methoxy-2,6-dimethylphenyl)-6-(trifluoromethyl) pyrido[3,4-d]pyrimidin-4-amine (39-9) (0.110 g, 0.316 mmol) in DCM (1.1 mL) was added BBr₃ (1 M solution in DCM, 1.26 mL, 1.26 mmol) at 0° C. The mixture was stirred at rt for 3 h. After completion, the reaction was quenched with saturated NaHCO₃ solution (50 mL) and extracted with EtOAc (2×100 mL). The combined organic layer was washed with brine (50 mL), dried over MgSO₄, filtered, and concentrated under reduced pressure. The residue was purified by column chromatography (SiO₂, MeOH/DCM gradient) to afford 3-(4-amino-6-(trifluoromethyl)pyrido[3,4-d]pyrimidin-8-yl)-2,4-dimethylphenol (39-A) (0.049 g, 46%). MS (ESI) m/z: 335.29 [M+H]⁺.

### Isolation of (S)-3-(4-amino-6-(trifluoromethyl) pyrido[3,4-d]pyrimidin-8-yl)-2,4-dimethylphenol (39-B) and (R)-3-(4-amino-6-(trifluoromethyl) pyrido[3,4-d]pyrimidin-8-yl)-2,4-dimethylphenol (39-C)

The racemic mixture of 3-(4-amino-6-(trifluoromethyl)pyrido[3,4-d]pyrimidin-8-yl)-2,4-dimethylphenol (39-A) (0.049 g) was subjected to chiral SFC purification (Chiralcel OJ-H, 30×250 mm column, CO₂/15% methanol) to afford the title compounds. From Peak 1: (S)-3-(4-amino-6-(trifluoromethyl) pyrido[3,4-d]pyrimidin-8-yl)-2,4-dimethylphenol (39-B) (0.011 g, 16.36%). Chiral purity 99.46% (Chiralcel OJ-H, 30×250 mm column, CO₂/15% methanol); ¹H NMR (400 MHz, DMSO-d₆): δ ppm 9.19 (s, 1H), 8.78 (s, 1H), 8.56-8.40 (m, 3H), 6.91 (d, J=8.0 Hz, 1H), 6.80 (d, J=8.0 Hz, 1H), 1.70 (s, 3H), 1.61 (s, 3H). MS (ESI) m/z: 335.29 [M+H]⁺. From Peak 2: (R)-3-(4-amino-6-(trifluoromethyl) pyrido[3,4-d]pyrimidin-8-yl)-2,4-dimethylphenol (39-C) (0.016 g, 21.82%). Chiral purity 98.54% (Chiralcel OJ-H, 30×250 mm column, CO₂/15% methanol); ¹H NMR (400 MHz, DMSO-d₆): δ ppm 9.19 (s, 1H), 8.78 (s, 1H), 8.56-8.40 (m, 3H), 6.91 (d, J=8.00 Hz, 1H), 6.80 (d, J=8.0 Hz, 1H), 1.70 (s, 3H), 1.62 (s, 3H). MS (ESI) m/z: 335.29 [M+H]⁺. Compounds 39-B and 39-C are atropisomers, and the shown stereochemistry may be the opposite of that depicted.

### Example 40

### (S)-3-(4-amino-6-cyclobutylpyrido[3,4-d]pyrimidin-8-yl)-2,4-dimethylphenol (40-B) and (R)-3-(4-amino-6-cyclobutylpyrido[3,4-d]pyrimidin-8-yl)-2,4-dimethylphenol (40-C)

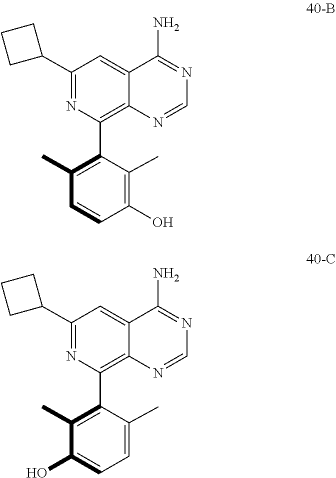

The racemic mixture of 3-(4-amino-6-cyclobutylpyrido[3,4-d]pyrimidin-8-yl)-2,4-dimethylphenol (40-A) (0.090 g) was prepared according to the procedure described in Example 19 and was subjected to chiral SFC purification (Chiralcel OJ-H, 4.6×250 mm column, CO₂/15% methanol) to afford the title compounds. From Peak 1: (S)-3-(4-amino-6-cyclobutylpyrido[3,4-d]pyrimidin-8-yl)-2,4-dimethylphenol (40-B) (0.033 g, 8.83%). Chiral purity 99.52% (Chiralcel OJ-H, 4.6×250 mm column, CO₂/15% methanol); ¹H NMR (400 MHz, DMSO-d₆): δ ppm 9.06 (s, 1H), 8.30 (s, 1H), 8.03 (brs, 2H), 7.94 (s, 1H), 6.87 (d, J=8.0 Hz, 1H), 6.75 (d, J=8.0 Hz, 1H), 3.80-3.71 (m, 1H), 2.35-2.29 (m, 4H), 2.08-1.99 (m, 1H), 1.88-1.82 (m, 1H), 1.68 (s, 3H), 1.60 (s, 3H). MS (ESI) m/z: 321.33 [M+H]⁺. From Peak 2: (R)-3-(4-amino-6-cyclobutylpyrido[3,4-d]pyrimidin-8-yl)-2,4-dimethylphenol (40-C) (0.035 g, 9.37%). Chiral purity 98.64% (Chiralcel OJ-H, 4.6×250 mm column, CO₂/15% methanol); ¹H NMR (400 MHz, DMSO-d₆): δ ppm 9.06 (s, 1H), 8.30 (s, 1H), 8.03 (brs, 2H), 7.94 (s, 1H), 6.87 (d, J=8.0 Hz, 1H), 6.75 (d, J=8.0 Hz, 1H), 3.80-3.71 (m, 1H), 2.35-2.29 (m, 4H), 2.08-2.04 (m, 1H), 1.90-1.82 (m, 1H), 1.68 (s, 3H), 1.60 (s, 3H). MS (ESI) m/z: 321.33 [M+H]⁺. Compounds 40-B and 40-C are atropisomers, and the shown stereochemistry may be the opposite of that depicted.

### Example 41

### (S)-3-((4-Amino-8-(3-hydroxy-2,6-dimethylphenyl)pyrido[3,4-d]pyrimidin-6-yl)(methyl)amino)propanenitrile (41-B) and (R)-3-((4-amino-8-(3-hydroxy-2,6-dimethylphenyl)pyrido[3,4-d]pyrimidin-6-yl)(methyl)amino)propanenitrile (41-C)

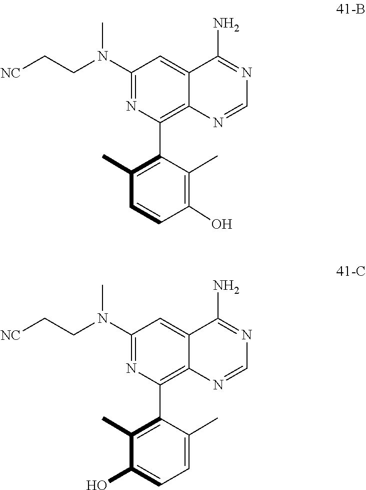

The racemic mixture of 3-((4-amino-8-(3-hydroxy-2,6-dimethylphenyl)pyrido[3,4-d]pyrimidin-6-yl)(methyl)amino)propanenitrile (41-A) (70 mg) was prepared according to the procedure described in Example 29 and was subjected to chiral SFC purification (Chiralcel-OJ-H, 30×250 mmm column, 15% ACN/MeOH (1:1) with 0.1% 7N NH₃ in MeOH) to afford the title compounds. From Peak 1: (S)-3-((4-amino-8-(3-hydroxy-2,6-dimethylphenyl)pyrido[3,4-d]pyrimidin-6-yl)(methyl)amino)propanenitrile (41-B) (13.72 mg, 20%). Chiral purity 99.2% (Chiralcel OJ-H (4.6×250 mm column), 15% ACN/MeOH (1:1) with 0.2% 7N NH₃ in MeOH); ¹H NMR (400 MHz, DMSO-d₆): δ ppm 9.05 (s, 1H), 8.06 (s, 1H), 7.82 (br. s, 2H), 7.11 (s, 1H), 6.86 (d, J=8.0 Hz, 1H), 6.74 (d, J=8.0 Hz, 1H), 3.90 (t, J=6.6 Hz, 2H), 3.13 (s, 3H), 2.75 (t, J=6.6 Hz, 2H), 1.72 (s, 3H), 1.64 (s, 3H); MS (ESI) m/z: 349.23 [M+H]⁺. From Peak 2: (R)-3-((4-amino-8-(3-hydroxy-2,6-dimethylphenyl)pyrido[3,4-d]pyrimidin-6-yl)(methyl)amino)propanenitrile (41-C) (12.69 mg, 19%). Chiral purity 97.65% (Chiralcel OJ-H (4.6×250 mm column), 15% ACN/MeOH (1:1) with 0.2% 7N NH₃ in MeOH); ¹H NMR (400 MHz, DMSO-d₆): δ ppm 9.03 (s, 1H), 8.05 (s, 1H), 7.80 (br. s, 2H), 7.10 (s, 1H), 6.85 (d, J=8.0 Hz, 1H), 6.74 (d, J=8.4 Hz, 1H), 3.90 (t, J=6.6 Hz, 2H), 3.13 (s, 3H), 2.75 (t, J=6.6 Hz, 2H), 1.72 (s, 3H), 1.63 (s, 3H). MS (ESI) m/z: 349.2 [M+H]⁺. Compounds 41-1 and 41-2 are atropisomers, and the shown stereochemistry may be the opposite of that depicted.

### Example 42

### (S)-3-(4-Amino-6-(methyl(1-(trifluoromethyl)cyclopropyl)amino)pyrido[3,4-d]pyrimidin-8-yl)-2,4-dimethylphenol (42-B) and (R)-3-(4-amino-6-(methyl(1-(trifluoromethyl)cyclopropyl)amino)pyrido[3,4-d]pyrimidin-8-yl)-2,4-dimethylphenol (42-C)

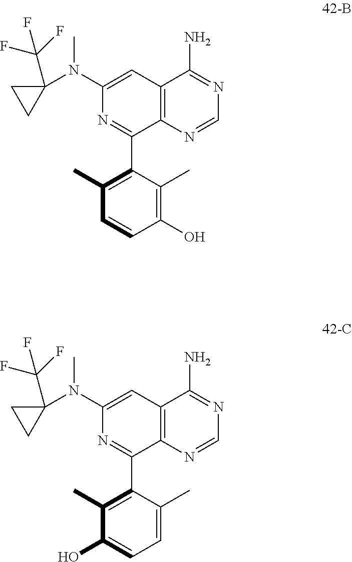

The racemic mixture of 3-(4-amino-6-(methyl(1-(trifluoromethyl)cyclopropyl)amino)pyrido[3,4-d]pyrimidin-8-yl)-2,4-dimethylphenol (42-A) (0.10 g) was prepared according to the procedure described in Example 28 and was subjected to chiral SFC purification (Chiralcel OJ-H, 30×250 mm column, 17% ACN/IPA (1:1) with 0.2% DEA) to afford the title compounds. From Peak 1: (S)-3-(4-amino-6-(methyl(1-(trifluoromethyl)cyclopropyl)amino)pyrido[3,4-d]pyrimidin-8-yl)-2,4-dimethylphenol (42-B) (35 mg, 35%). Chiral purity 99.7% (Chiralcel OJ-H (4.6×250 mm column), 20% ACN/IPA (1:1) with 0.2% DEA); ¹H NMR (400 MHz, DMSO-d₆): δ ppm 9.04 (s, 1H), 8.09 (s, 1H), 7.91 (br. s, 2H), 7.24 (s, 1H), 6.85 (d, J=8.0 Hz, 1H), 6.73 (d, J=8.0 Hz, 1H), 3.15 (s, 3H), 1.80-1.70 (m, 4H), 1.62 (s, 3H), 1.35-1.25 (m, 3H). MS (ESI) m/z: 404.3 [M+H]⁺. From Peak 2: (R)-3-(4-amino-6-(methyl(1-(trifluoromethyl)cyclopropyl)amino)pyrido[3,4-d]pyrimidin-8-yl)-2,4-dimethylphenol (42-C) (31 mg, 31%). Chiral purity 98.9% (Chiralcel OJ-H (4.6×250 mm column), 20% ACN/IPA with 0.2% DEA); ¹H NMR (400 MHz, DMSO-d₆): δ ppm 9.04 (s, 1H), 8.09 (s, 1H), 7.91 (br. s, 2H), 7.24 (s, 1H), 6.85 (d, J=8.0 Hz, 1H), 6.73 (d, J=8.4 Hz, 1H), 3.14 (s, 3H), 1.77-1.67 (m, 4H), 1.62 (s, 3H), 1.40-1.25 (m, 3H). MS (ESI) m/z: 404.2 [M+H]⁺. Compounds 42-B and 42-C are atropisomers, and the shown stereochemistry may be the opposite of that depicted.

### Example 43

### (S)-1-(4-amino-8-(3-hydroxy-2,6-dimethylphenyl)pyrido[3,4-d]pyrimidin-6-yl)pyrrolidin-2-one (43-B) and (R)-1-(4-amino-8-(3-hydroxy-2,6-dimethylphenyl)pyrido[3,4-d]pyrimidin-6-yl)pyrrolidin-2-one (43-C)

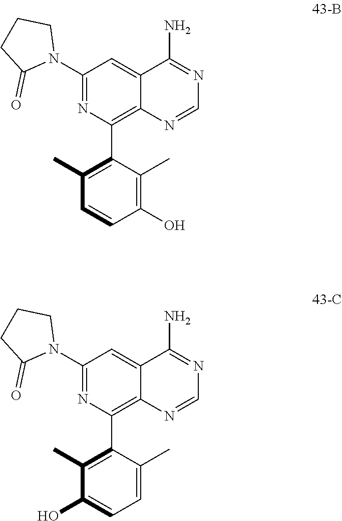

The racemic mixture of 1-(4-amino-8-(3-hydroxy-2,6-dimethylphenyl)pyrido[3,4-d]pyrimidin-6-yl)pyrrolidin-2-one (43-A) (0.05 g) was prepared according to the procedure described in Example 25 and was subjected to chiral SFC purification (Chiralcel OJ-H, 30×250 mm column, 15% ACN/MeOH (1:1) with 0.2% 7N NH₃ in MeOH) to afford the title compounds. From Peak 1: (S)-1-(4-amino-8-(3-hydroxy-2,6-dimethylphenyl)pyrido[3,4-d]pyrimidin-6-yl)pyrrolidin-2-one (43-B) (15 mg, 30%). Chiral purity 99.8% (Chiralcel OJ-H (4.6×250 mm column), 15% ACN/MeOH (1:1) with 0.2% 7N NH₃ in MeOH); ¹H NMR (400 MHz, DMSO-d₆): δ ppm 9.10 (s, 1H), 8.70 (s, 1H), 8.26 (s, 1H), 8.09 (br. s, 2H), 6.88 (d, J=8.4 Hz, 1H), 6.77 (d, J=8.0 Hz, 1H), 3.99 (t, J=7.0 Hz, 2H), 2.61 (t, J=8.0 Hz, 2H), 2.10-2.00 (m, 2H), 1.72 (s, 3H), 1.63 (s, 3H). MS (ESI) m/z: 350.3 [M+H]⁺. From Peak 2: (R)-1-(4-amino-8-(3-hydroxy-2,6-dimethylphenyl)pyrido[3,4-d]pyrimidin-6-yl)pyrrolidin-2-one (43-C) (12 mg, 24%). Chiral purity 98.4% (Chiralcel OJ-H (4.6×250 mm column), 15% ACN/MeOH (1:1) with 0.2% 7N NH₃ in MeOH); ¹H NMR (400 MHz, DMSO-d₆): δ ppm 9.11 (s, 1H), 8.70 (s, 1H), 8.26 (s, 1H), 8.08 (br. s, 2H), 6.88 (d, J=8.0 Hz, 1H), 6.77 (d, J=8.00 Hz, 1H), 3.99 (t, J=7.0 Hz, 2H), 2.61 (t, J=8.0 Hz, 2H), 2.11-2.01 (m, 2H), 1.72 (s, 3H), 1.63 (s, 3H). MS (ESI) m/z: 350.3 [M+H]⁺. Compounds 43-B and 43-C are atropisomers, and the shown stereochemistry may be the opposite of that depicted.

### Example 44

### (S)-3-(4-amino-6-(cyclopentyl(methyl)amino)pyrido[3,4-d]pyrimidin-8-yl)-2,4-dimethylphenol (44-B) and (R)-3-(4-amino-6-(cyclopentyl(methyl)amino)pyrido[3,4-d]pyrimidin-8-yl)-2,4-dimethylphenol (44-C)

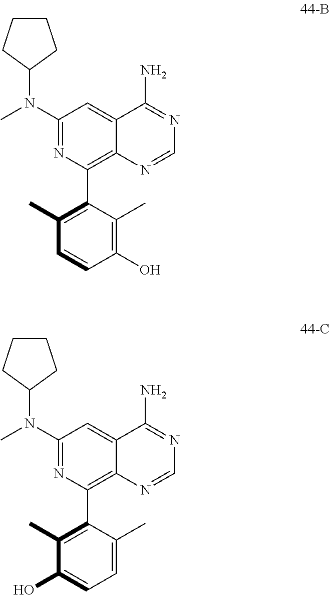

The racemic mixture of 3-(4-amino-6-(cyclopentyl(methyl)amino)pyrido[3,4-d]pyrimidin-8-yl)-2,4-dimethylphenol (44-A) (0.08 g) was prepared according to the procedure described in Example 37 and was subjected to chiral SFC purification (Chiralcel OJ-H, 30×250 mm column, 13% ACN/MeOH with 0.3% 7N NH₃ in MeOH) to afford to afford the title compounds. From Peak 1: (S)-3-(4-amino-6-(cyclopentyl(methyl)amino)pyrido[3,4-d]pyrimidin-8-yl)-2,4-dimethylphenol (44-B) (20 mg, 25%). Chiral purity 88.8% (Chiralcel OJ-H (4.6×250 mm column), 15% ACN/MeOH with 0.2% 7N NH₃ in MeOH); ¹H NMR (400 MHz, DMSO-d₆): δ ppm 9.03 (s, 1H), 8.01 (s, 1H), 7.73 (br. s, 2H), 7.01 (s, 1H), 6.85 (d, J=8.4 Hz, 1H), 6.73 (d, J=8.4 Hz, 1H), 5.07-4.96 (m, 1H), 2.89 (s, 3H), 1.84-1.65 (m, 7H), 1.65-1.50 (m, 7H); MS (ESI) m/z: 364.3 [M+H]⁺. From Peak 2: (R)-3-(4-amino-6-(cyclopentyl(methyl)amino)pyrido[3,4-d]pyrimidin-8-yl)-2,4-dimethylphenol (44-C) (12 mg, 15%). Chiral purity 77.4% (Chiralcel OJ-H (4.6×250 mm column), 15% ACN/MeOH (1:1) with 0.2% 7N NH₃ in MeOH); ¹H NMR (400 MHz, DMSO-d₆): δ ppm 9.03 (s, 1H), 8.01 (s, 1H), 7.73 (br. s, 2H), 7.01 (s, 1H), 6.85 (d, J=8.0 Hz, 1H), 6.73 (d, J=8.0 Hz, 1H), 5.08-4.97 (m, 1H), 2.89 (s, 3H), 1.85-1.65 (m, 7H), 1.65-1.50 (m, 7H); MS (ESI) m/z: 364.3 [M+H]⁺. Compounds 44-B and 44-C are atropisomers, and the shown stereochemistry may be the opposite of that depicted.

### Example 45

### 3-((S)-4-Amino-6-(((S)-sec-butyl)(methyl)amino)pyrido[3,4-d]pyrimidin-8-yl)-2,4-dimethylphenol (45-B) and 3-((R)-4-amino-6-(((S)-sec-butyl)(methyl)amino)pyrido[3,4-d]pyrimidin-8-yl)-2,4-dimethylphenol (45-C)

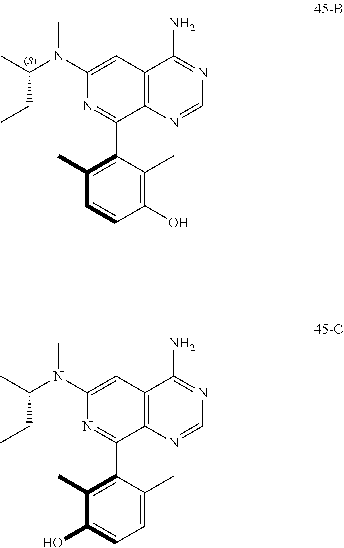

The racemic mixture of 3-(4-amino-6-(((S)-sec-butyl)(methyl)amino)pyrido[3,4-d]pyrimidin-8-yl)-2,4-dimethylphenol (45-A) (41 mg) was prepared according to the procedure described in Example 37 (replacing Pd-PEPPSI-iPent-Cl with Pd(OAc)₂/BINAP in the first step) and was subjected to chiral SFC purification (Chiralpak IH, 30×250 mm column, 22% IPA with 0.2% ⁱPrNH₂) to afford to afford the title compounds. From Peak 2: 3-((S)-4-amino-6-(((S)-sec-butyl)(methyl)amino)pyrido[3,4-d]pyrimidin-8-yl)-2,4-dimethylphenol (45-B) (10 mg, 24%). Chiral purity 98.1% (Chiralpak IH (4.6×250 mm column), 25% IPA with 0.2% PrNH₂); ¹H NMR (400 MHz, DMSO-d₆): δ ppm δ 9.02 (s, 1H), 8.00 (s, 1H), 7.71 (br. s, 2H), 6.97 (s, 1H), 6.85 (d, J=8.0 Hz, 1H), 6.73 (d, J=8.4 Hz, 1H), 4.88-4.76 (m, 1H), 2.82 (s, 3H), 1.72 (s, 3H), 1.64 (s, 3H), 1.60-1.42 (m, 2H), 1.08 (d, J=6.4 Hz, 3H), 0.74 (t, J=7.2 Hz, 3H). MS (ESI) m/z: 352.3 [M+H]⁺. From Peak 2: 3-((R)-4-amino-6-(((S)-sec-butyl)(methyl)amino)pyrido[3,4-d]pyrimidin-8-yl)-2,4-dimethylphenol (45-C) (11 mg, 26%). Chiral purity 97.2% (Chiralpak IH (4.6×250 mm column), 25% IPA with 0.2% PrNH₂); ¹H NMR (400 MHz, DMSO-d₆): δ ppm δ 9.03 (s, 1H), 8.00 (s, 1H), 7.71 (br. s, 2H), 6.97 (s, 1H), 6.85 (d, J=8.0 Hz, 1H), 6.73 (d, J=8.4 Hz, 1H), 4.87-4.75 (m, 1H), 2.83 (s, 3H), 1.73 (s, 3H), 1.63 (s, 3H), 1.60-1.41 (m, 2H), 1.08 (d, J=6.4 Hz, 3H), 0.74 (t, J=7.2 Hz, 3H). MS (ESI) m/z: 352.3 [M+H]⁺. Compounds 45-B and 45-C are atropisomers, and the shown stereochemistry may be the opposite of that depicted.

### Example 46

### (S)-3-(4-Amino-6-(methyl(1-methyl-1H-pyrazol-4-yl)amino)pyrido[3,4-d]pyrimidin-8-yl)-2,4-dimethylphenol (46-B) and (R)-3-(4-amino-6-(methyl(1-methyl-1H-pyrazol-4-yl)amino)pyrido[3,4-d]pyrimidin-8-yl)-2,4-dimethylphenol (46-C)

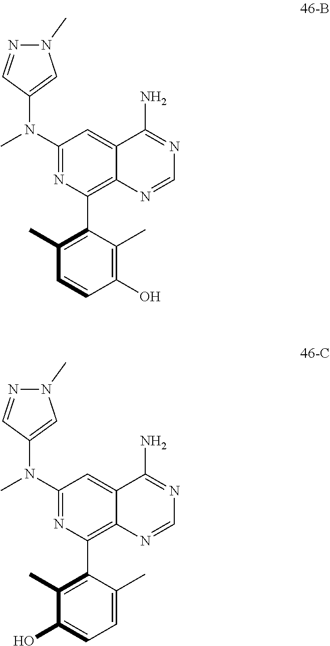

The racemic mixture of 3-(4-amino-6-(methyl(1-methyl-1H-pyrazol-4-yl)amino)pyrido[3,4-d]pyrimidin-8-yl)-2,4-dimethylphenol (46-A) (0.09 g) was prepared according to the procedure described in Example 37 (replacing Cs₂CO₃ with K₃PO₄, and Pd-PEPPSI-iPent-Cl with Pd₂dba₃/Xantphos in the first step) and was subjected to chiral SFC purification (Chiralpak IH, 27% IPA with 0.1% ⁱPrNH₂) to afford the title compounds. From Peak 1: (S)-3-(4-amino-6-(methyl(1-methyl-1H-pyrazol-4-yl)amino)pyrido[3,4-d]pyrimidin-8-yl)-2,4-dimethylphenol (46-B) (28 mg, 33%). Chiral purity 98.6% (Chiralpak IH (4.6×250 mm column), 30% IPA with 0.2% PrNH₂); ¹H NMR (400 MHz, DMSO-d₆): δ ppm 9.05 (s, 1H), 8.07 (s, 1H), 7.85-7.75 (m, 3H), 7.54 (s, 1H), 7.22 (s, 1H), 6.86 (d, J=8.0 Hz, 1H), 6.74 (d, J=8.0 Hz, 1H), 3.80 (s, 3H), 3.35 (s, 3H), 1.74 (s, 3H), 1.65 (s, 3H). MS (ESI) m/z: 376.4 [M+H]⁺. From Peak 2: (R)-3-(4-amino-6-(methyl(1-methyl-1H-pyrazol-4-yl)amino)pyrido[3,4-d]pyrimidin-8-yl)-2,4-dimethylphenol, (46-C) (31 mg, 36%). Chiral purity 96.0% (Chiralpak IH (4.6×250 mm column), 30% IPA with 00.2% ⁱPrNH₂); ¹H NMR (400 MHz, DMSO-d₆): δ ppm 9.05 (s, 1H), 8.07 (s, 1H), 7.85-7.75 (m, 3H), 7.54 (s, 1H), 7.22 (s, 1H), 6.86 (d, J=8.4 Hz, 1H), 6.74 (d, J=8.0 Hz, 1H), 3.80 (s, 3H), 3.35 (s, 3H), 1.74 (s, 3H), 1.65 (s, 3H). MS (ESI) m/z: 376.4 [M+H]⁺. Compounds 46-B and 46-C are atropisomers, and the shown stereochemistry may be the opposite of that depicted.

### Example 47

### (S)-3-(4-Amino-6-((2-hydroxyethyl)(methyl)amino)pyrido[3,4-d]pyrimidin-8-yl)-2,4-dimethylphenol (47-B) and (R)-3-(4-Amino-6-((2-hydroxyethyl)(methyl)amino)pyrido[3,4-d]pyrimidin-8-yl)-2,4-dimethylphenol (47-C)

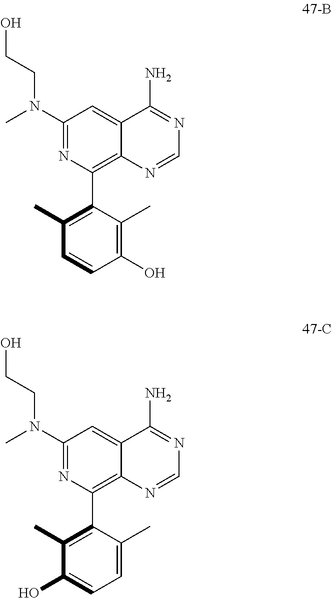

The racemic mixture of 3-(4-amino-6-((2-hydroxyethyl)(methyl)amino)pyrido[3,4-d]pyrimidin-8-yl)-2,4-dimethylphenol (47-A) was prepared following the procedure described in Example 1 and was purified by prep-HPLC (X bridge C18, 19×150 mm, ACN/H₂O/10 mM NH₄HCO₃) to afford racemic 3-(4-amino-6-((2-hydroxyethyl)(methyl)amino)pyrido[3,4-d]pyrimidin-8-yl)-2,4-dimethylphenol (47-A) (6.1 mg, 3%). ¹H NMR (400 MHz, DMSO-d₆): δ ppm 9.02 (br. s, 1H), 8.00 (s, 1H), 7.72 (br. s, 2H), 6.98 (s, 1H), 6.85 (d, J=8.0 Hz, 1H), 6.73 (d, J=8.4 Hz, 1H), 4.68-4.60 (m, 1H), 3.69-3.52 (m, 4H), 3.10 (s, 3H), 1.72 (s, 3H), 1.63 (s, 3H). MS (ESI) m/z: 340.4 [M+H]⁺. The racemic mixture of 3-(4-amino-6-((2-hydroxyethyl)(methyl)amino)pyrido[3,4-d]pyrimidin-8-yl)-2,4-dimethylphenol (47-A) can be subjected to chiral SFC purification (Chiralpak IH, 30×250 mm column, 22% IPA with 0.2% ⁱPrNH₂) to afford to afford the title compounds.

### Example 48

### (S)-3-(4-amino-6-(difluoromethyl)pyrido[3,4-d]pyrimidin-8-yl)-2,4-dimethylphenol (48-B) and (R)-3-(4-amino-6-(difluoromethyl)pyrido[3,4-d]pyrimidin-8-yl)-2,4-dimethylphenol (48-C)

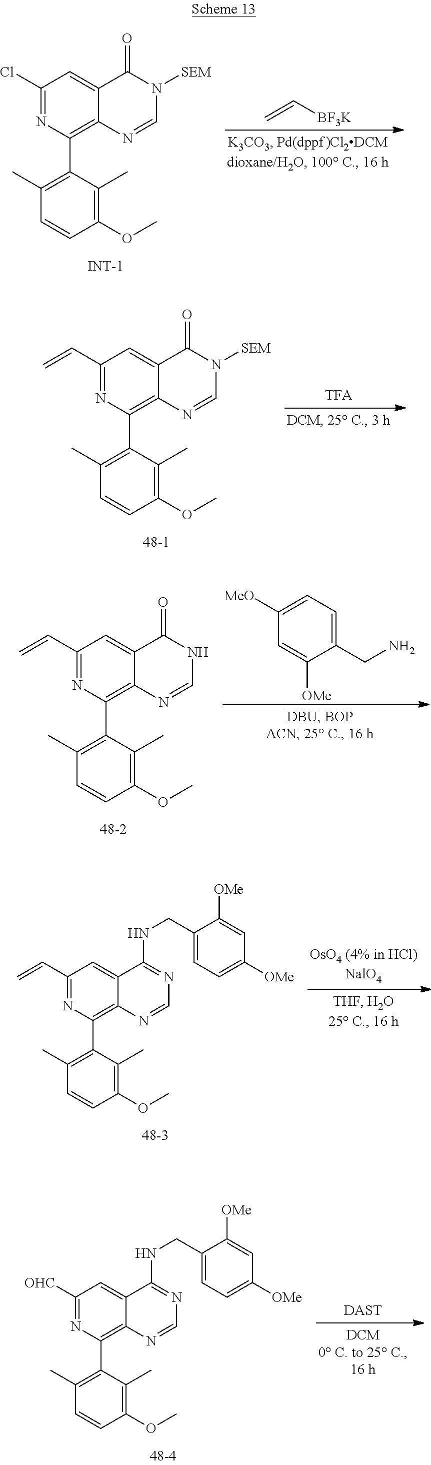

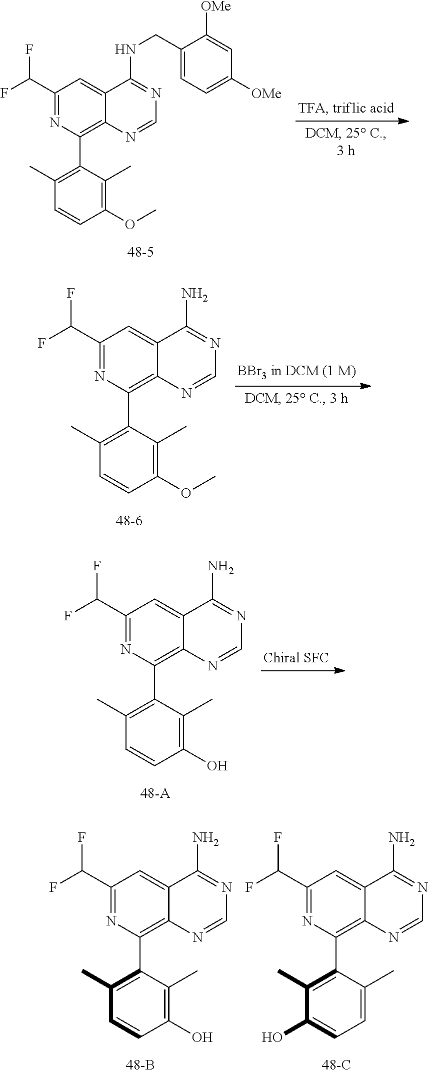

### 8-(3-methoxy-2,6-dimethylphenyl)-3-((2-(trimethylsilyl)ethoxy)methyl)-6-vinylpyrido[3,4-d]pyrimidin-4 (3H)-one (48-1)

To a solution of 6-chloro-8-(3-methoxy-2,6-dimethylphenyl)-3-((2-(trimethylsilyl)ethoxy)methyl)pyrido[3,4-d]pyrimidin-4 (3H)-one (INT-1) (2.0 g, 4.48 mmol) and potassium trifluoro(vinyl)borate (0.721 g, 5.381 mmol) in a mixture of 1,4-dioxane (40 mL) and water (10 mL) was added K₂CO₃ (0.930 g, 6.726 mmol) at 25° C. The mixture was degassed with N₂ for 15 min followed by addition of (1,1′-bis(diphenylphosphino)ferrocene)dichloropalladium-dichloromethane (0.183 g, 0.224 mmol). The mixture was degassed again with N₂ for another 10 min and stirred at 100° C. for 16 h. After completion, the reaction was quenched with water (60 mL) and extracted with EtOAc (2×60 mL). The combined organic layer was washed with brine (60 mL), dried over MgSO₄, filtered, and concentrated. The residue was purified by column chromatography (SiO₂, EtOAc/PET) to afford 8-(3-methoxy-2,6-dimethylphenyl)-3-((2-(trimethylsilyl)ethoxy)methyl)-6-vinylpyrido[3,4-d]pyrimidin-4 (3H)-one (48-1) (1.0 g, 50%). MS (ESI) m/z: 438.76 [M+H]⁺.

### 8-(3-methoxy-2,6-dimethylphenyl)-6-vinylpyrido[3,4-d]pyrimidin-4 (3H)-one (48-2)

To a solution of 8-(3-methoxy-2,6-dimethylphenyl)-3-((2-(trimethylsilyl)ethoxy)methyl)-6-vinylpyrido[3,4-d]pyrimidin-4 (3H)-one (48-1) (1.0 g, 2.285 mmol) in DCM (10.0 mL) was added TFA (5.0 mL) at 25° C. The mixture was stirred at 25° C. for 3 h. After completion, the reaction was quenched with saturated NaHCO₃ (80 mL) and extracted with EtOAc (2×80 mL). The combined organic layer was washed with brine (80 mL), dried over MgSO₄, filtered, and concentrated. The residue was purified by column chromatography (SiO₂, EtOAc/PET) to afford 8-(3-methoxy-2,6-dimethylphenyl)-6-vinylpyrido[3,4-d]pyrimidin-4 (3H)-one (48-2) (0.57 g, 81%). MS (ESI) m/z: 308.57 [M+H]⁺.

### N-(2,4-dimethoxybenzyl)-8-(3-methoxy-2,6-dimethylphenyl)-6-vinylpyrido[3,4-d]pyrimidin-4-amine (48-3)

To a solution of 8-(3-methoxy-2,6-dimethylphenyl)-6-vinylpyrido[3,4-d]pyrimidin-4 (3H)-one (48-2) (0.565 g, 1.838 mmol) in ACN (5.7 mL) were added (2,4-dimethoxyphenyl)methanamine (0.615 g, 3.677 mmol), DBU (0.55 mL, 3.677 mmol), and BOP (1.22 g, 2.757 mmol) at 25° C. The mixture was stirred at 25° C. for 16 h. After completion, the reaction was quenched with water (60 mL) and extracted with EtOAc (3×60 mL). The combined organic layer was washed with brine (30 mL), dried over MgSO₄, filtered, and concentrated. The residue was purified by column chromatography (SiO₂, EtOAc/PET) to afford N-(2,4-dimethoxybenzyl)-8-(3-methoxy-2,6-dimethylphenyl)-6-vinylpyrido[3,4-d]pyrimidin-4-amine (48-3) (0.540 g, 64%). MS (ESI) m/z: 457.80 [M+H]⁺.

### 4-((2,4-dimethoxybenzyl)amino)-8-(3-methoxy-2,6-dimethylphenyl)pyrido[3,4-d]pyrimidine-6-carbaldehyde (48-4)

To a solution of N-(2,4-dimethoxybenzyl)-8-(3-methoxy-2,6-dimethylphenyl)-6-vinylpyrido[3,4-d]pyrimidin-4-amine (48-3) (0.5 g, 1.095 mmol) in THF (6.0 mL) and water (1.2 mL) was added OsO₄ (4 wt. % solution in water, 27.839 mg, 0.110 mmol) at 25° C. The mixture was stirred at 25° C. for 5 min and NaIO₄ (0.703 g, 3.286 mmol) was added. The mixture was stirred at 25° C. for 16 h. After completion, the reaction was quenched with saturated NaHCO₃ (60 mL) and extracted with EtOAc (2×60 mL). The combined organic layer was washed with brine (50 mL), dried over MgSO₄, filtered, and concentrated. The residue was purified by column chromatography (SiO₂, EtOAc/PET) to afford 4-((2,4-dimethoxybenzyl)amino)-8-(3-methoxy-2,6-dimethylphenyl)pyrido[3,4-d]pyrimidine-6-carbaldehyde (48-4) (0.240 g, 47.83%). MS (ESI) m/z: 459.50 [M+H]⁺.

### 6-(difluoromethyl)-N-(2,4-dimethoxybenzyl)-8-(3-methoxy-2,6-dimethylphenyl)pyrido[3,4-d]pyrimidin-4-amine (48-5)

To a solution of 4-((2,4-dimethoxybenzyl)amino)-8-(3-methoxy-2,6-dimethylphenyl)pyrido[3,4-d]pyrimidine-6-carbaldehyde (48-4) (0.46 g, 1.003 mmol) in DCM (4.6 mL) was added DAST (0.331 mL, 2.508 mmol) at 0° C. The mixture was stirred at 25° C. for 16 h. After completion, the reaction was quenched with saturated NaHCO₃ (60 mL) and extracted with EtOAc (2×60 mL). The combined organic layer was washed with brine (50 mL), dried over MgSO₄, filtered, and concentrated. The residue was purified by column chromatography (SiO₂, EtOAc/PET) to afford 6-(difluoromethyl)-N-(2,4-dimethoxybenzyl)-8-(3-methoxy-2,6-dimethylphenyl)pyrido[3,4-d]pyrimidin-4-amine (48-5) (0.24 g, 48%). MS (ESI) m/z: 481.50 [M+H]⁺.

### 6-(difluoromethyl)-8-(3-methoxy-2,6-dimethylphenyl)pyrido[3,4-d]pyrimidin-4-amine (48-6)

To a solution of 6-(difluoromethyl)-N-(2,4-dimethoxybenzyl)-8-(3-methoxy-2,6-dimethylphenyl)pyrido[3,4-d]pyrimidin-4-amine (48-5) (0.12 g, 0.25 mmol) in DCM (1.2 mL) were added TFA (0.6 mL) and triflic acid (0.120 mL) at 0° C. The mixture was stirred at 25° C. for 3 h. After completion, the reaction was quenched with saturated NaHCO₃ (50 mL) and extracted with EtOAc (2×50 mL). The combined organic layer was washed with brine (50 mL), dried over MgSO₄, filtered, and concentrated to afford 6-(difluoromethyl)-8-(3-methoxy-2,6-dimethylphenyl)pyrido[3,4-d]pyrimidin-4-amine (48-6) (0.08 g). MS (ESI) m/z: 331.56 [M+H]⁺. The crude was used for the next step without purification.

### 3-(4-amino-6-(difluoromethyl)pyrido[3,4-d]pyrimidin-8-yl)-2,4-dimethylphenol (48-A)

To a solution of 6-(difluoromethyl)-8-(3-methoxy-2,6-dimethylphenyl)pyrido[3,4-d]pyrimidin-4-amine (48-6) (0.08 g, 0.242 mmol) in DCM (0.8 mL) was added BBr₃ (1M in DCM, 0.097 mL, 0.097 mmol) at 0° C. The mixture was stirred at 25° C. for 3 h. After completion, the reaction was quenched with saturated NaHCO₃ (50 mL) and extracted with EtOAc (2×50 mL). The combined organic layer was washed with brine (50 mL), dried over MgSO₄, filtered, and concentrated. The residue was purified by column chromatography (SiO₂, MeOH/DCM) to afford racemic 3-(4-amino-6-(difluoromethyl)pyrido[3,4-d]pyrimidin-8-yl)-2,4-dimethylphenol (48-A) (0.024 g, 31%). MS (ESI) m/z: 317.53 [M+H]⁺.

### Isolation of (S)-3-(4-amino-6-(difluoromethyl)pyrido[3,4-d]pyrimidin-8-yl)-2,4-dimethylphenol (48-B) and (R)-3-(4-amino-6-(difluoromethyl)pyrido[3,4-d]pyrimidin-8-yl)-2,4-dimethylphenol (48-C)

The racemic mixture of 3-(4-amino-6-(difluoromethyl)pyrido[3,4-d]pyrimidin-8-yl)-2,4-dimethylphenol (0.024 g) was subjected to chiral SFC purification (CHIRALPAK-IH, 30×250 mm column, CO₂/20% methanol) to afford the title compounds. From Peak 1: (S)-3-(4-amino-6-(difluoromethyl)pyrido[3,4-d]pyrimidin-8-yl)-2,4-dimethylphenol (48-B) (0.01 g, 40%). Chiral purity 99.92% (CHIRALPAK-IH, 30×250 mm column, CO₂/20% methanol); ¹H NMR (400 MHz, DMSO-d₆): δ ppm 9.13 (s, 1H), 8.53-8.28 (m, 4H), 7.075 (t, J=5.52 Hz, 1H), 6.89 (d, J=8.4 Hz, 1H), 6.78 (d, J=8.0 Hz, 1H), 1.68 (s, 3H), 1.60 (s, 3H). MS (ESI) m/z: 317.19 [M+H]⁺. From Peak 2: (R)-3-(4-amino-6-(difluoromethyl)pyrido[3,4-d]pyrimidin-8-yl)-2,4-dimethylphenol (48-C) (0.008 g, 32%). Chiral purity 97.4% (CHIRALPAK-IH, 30×250 mm column, CO₂/20% methanol); ¹H NMR (400 MHz, DMSO-d₆): δ ppm 9.13 (s, 1H), 8.53-8.27 (m, 4H), 7.075 (t, J=5.52 Hz, 1H), 6.89 (d, J=8.4 Hz, 1H), 6.78 (d, J=8.4 Hz, 1H), 1.68 (s, 3H), 1.60 (s, 3H). MS (ESI) m/z: 317.19 [M+H]⁺. Compounds 48-B and 48-C are atropisomers, and the shown stereochemistry may be the opposite of that depicted.

### Example 49

### (S)-3-(4-amino-6-methoxypyrido[3,4-d]pyrimidin-8-yl)-2,4-dimethylphenol (49-B) and (R)-3-(4-amino-6-methoxypyrido[3,4-d]pyrimidin-8-yl)-2,4-dimethylphenol (49-C)

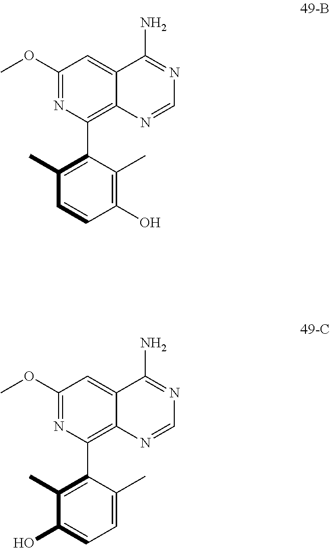

The racemic mixture of 3-(4-amino-6-methoxypyrido[3,4-d]pyrimidin-8-yl)-2,4-dimethylphenol (49-A) (44 mg) was prepared according to the procedure described in Example 6 (replacing DIPEA with NaOMe in MeOH) and was subjected to chiral SFC purification (Lux-i-AMYLOSE-3, 30×250 mm column, CO₂/27% methanol) to afford the title compounds. From Peak 1: (S)-3-(4-amino-6-methoxypyrido[3,4-d]pyrimidin-8-yl)-2,4-dimethylphenol (49-B) (0.0112 g, 5.8%). Chiral purity 99.84% (Lux-i-AMYLOSE-3, 30×250 mm column, CO₂/27% methanol); ¹H NMR (400 MHz, DMSO-d₆): δ ppm 9.10 (s, 1H), 8.19 (s, 1H), 7.99 (brs, 2H), 7.53 (s, 1H), 6.87 (d, J=8.4 Hz, 1H), 6.76 (d, J=8.0 Hz, 1H), 3.89 (s, 3H), 1.71 (s, 3H), 1.62 (s, 3H). MS (ESI) 297.30 [M+H]⁺. From Peak 2: (R)-3-(4-amino-6-methoxypyrido[3,4-d]pyrimidin-8-yl)-2,4-dimethylphenol (49-C) (0.0135 g, 9.4%). Chiral purity 98.58% (Lux-i-AMYLOSE-3, 30×250 mm column, CO₂/27% methanol); ¹H NMR (400 MHz, DMSO-d₆): δ ppm 9.10 (s, 1H), 8.19 (s, 1H), 7.98 (brs, 2H), 7.53 (s, 1H), 6.87 (d, J=8.0 Hz, 1H), 6.76 (d, J=8.0 Hz, 1H), 3.89 (s, 3H), 1.71 (s, 3H), 1.62 (s, 3H). MS (ESI) 297.30 [M+H]⁺. Compounds 49-B and 49-C are atropisomers, and the shown stereochemistry may be the opposite of that depicted.

### Example 50

### (S)-3-(4-amino-6-isopropylpyrido[3,4-d]pyrimidin-8-yl)-2,4-dimethylphenol (50-B) and (R)-3-(4-amino-6-isopropylpyrido[3,4-d]pyrimidin-8-yl)-2,4-dimethylphenol (50-C)

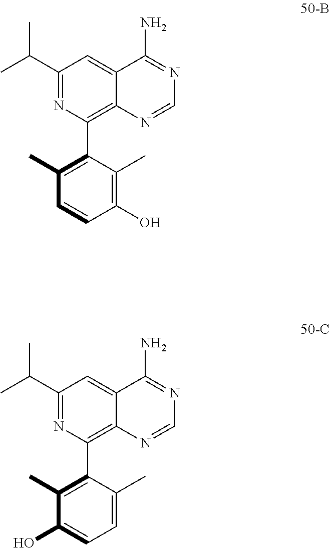

The racemic mixture of 3-(4-amino-6-isopropylpyrido[3,4-d]pyrimidin-8-yl)-2,4-dimethylphenol (50-A) (36 mg) was prepared according to the procedure described in Example 48 and was subjected to chiral SFC purification (Chiralcel OJ-H, 30×250 mm column, CO₂/13% methanol) to afford the title compounds. From Peak 1: (S)-3-(4-amino-6-isopropylpyrido[3,4-d]pyrimidin-8-yl)-2,4-dimethylphenol (50-B) (0.015 g, 41.6%). Chiral purity 99.50% (Chiralcel OJ-H, 30×250 mm column, CO₂/13% methanol); ¹H NMR (400 MHz, DMSO-d₆): δ ppm 9.06 (s, 1H), 8.30 (s, 1H), 8.03-7.95 (m, 3H), 6.87 (d, J=8.4 Hz, 1H), 6.75 (d, J=8.0 Hz, 1H), 3.14-3.09 (m, 1H), 1.68 (s, 3H), 1.60 (s, 3H), 1.32-1.30 (m, 6H). MS (ESI) m/z: 309.32 [M+H]⁺. From Peak 2: (R)-3-(4-amino-6-isopropylpyrido[3,4-d]pyrimidin-8-yl)-2,4-dimethylphenol (50-C) (0.018 g, 50%). Chiral purity 96.74% (Chiralcel OJ-H, 30×250 mm column, CO₂/13% methanol); ¹H NMR (400 MHz, DMSO-d₆): δ ppm 9.07 (s, 1H), 8.31 (s, 1H), 7.96 (br s, 3H), 6.87 (d, J=8.0 Hz, 1H), 6.75 (d, J=8.0 Hz, 1H), 3.17-3.09 (m, 1H), 1.68 (s, 3H), 1.60 (s, 3H), 1.32-1.23 (m, 6H). MS (ESI) m/z: 309.33 [M+H]⁺. Compounds 50-1 and 50-2 are atropisomers, and the shown stereochemistry may be the opposite of that depicted.

### Example 51

### (R)-3-(4-amino-6-(dimethylamino)-7-methylquinazolin-8-yl)-2,4-dimethylphenol (51-B) and (S)-3-(4-amino-6-(dimethylamino)-7-methylquinazolin-8-yl)-2,4-dimethylphenol

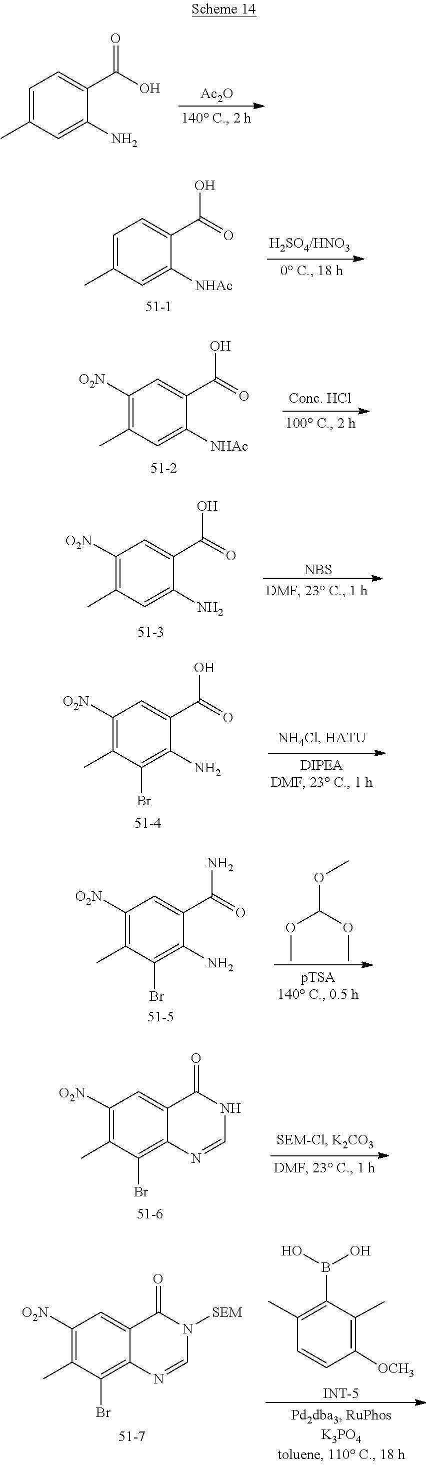

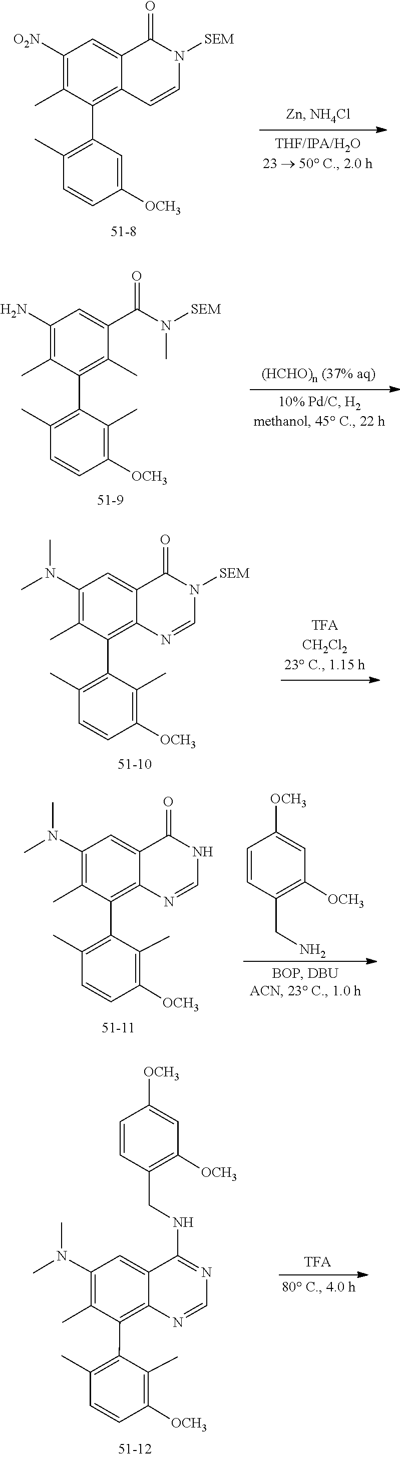 [SMILES: COc1ccc(C)c(-c2c(C)c([N+](=O)[O-])cc3c(=O)n(COCC[Si](C)(C)C)ccc23)c1 ; COc1ccc(C)c(-c2c(C)c(N)cc(C(=O)N(C)COCC[Si](C)(C)C)c2C)c1C ; COc1ccc(C)c(-c2c(C)c(N(C)C)cc3c(=O)n(COCC[Si](C)(C)C)cnc23)c1C ; COc1ccc(C)c(-c2c(C)c(N(C)C)cc3c(=O)[nH]cnc23)c1C ; COc1ccc(CN)c(OC)c1 ; COc1ccc(CNc2ncnc3c(-c4c(C)ccc(OC)c4C)c(C)c(N(C)C)cc23)c(OC)c1]

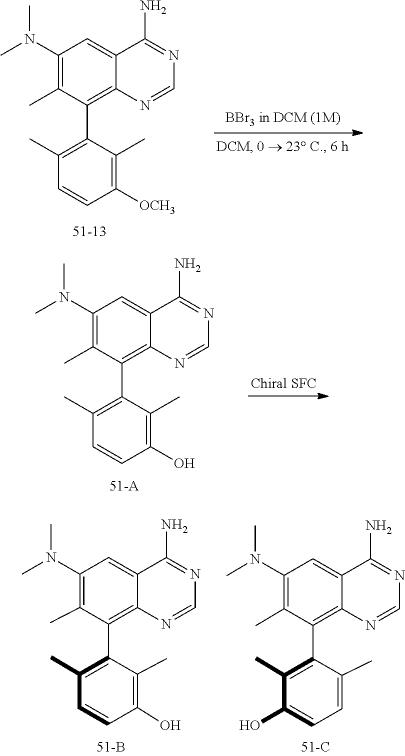

### 2-acetamido-4-methylbenzoic acid (51-1)

A solution of 2-amino-4-methylbenzoic acid (3.4 g, 22.49 mmol) in Ac₂O (6.3 mL) was stirred at 140° C. for 2 h. After completion, the mixture was cooled to rt, and cold water (50 mL) was added. The suspension was stirred at rt for an additional 2 h. The product was filtered and washed with water (300 mL), dissolved in acetone (300 mL), dried over Na₂SO₄, filtered, and concentrated to obtain 2-acetamido-4-methylbenzoic acid (51-1) (4.31 g, 99%), which was used for the next step without further purification. MS (APCI) m/z: 194.10 [M+H]⁺.

### 2-acetamido-4-methyl-5-nitrobenzoic acid (51-2)

A solution of 2-acetamido-4-methylbenzoic acid (51-1) (6.0 g, 31.1 mmol) in concentrated H₂SO₄ (79.8 mL) was cooled to 0° C. and stirred for 10 min. Concentrated HNO₃ (8.69 mL) was slowly added, and the mixture was stirred at 0° C. for 18 h. After completion, cold water (500 mL) was added to the mixture. The suspension was stirred at rt for an additional 2 h. The product was filtered and washed with water (500 mL), dissolved in acetone (300 mL), dried over Na₂SO₄, filtered, and concentrated to obtain 2-acetamido-4-methyl-5-nitrobenzoic acid (51-2) (5.9 g, 80%), which was used for the next step without further purification. MS (APCI) m/z: 237.10 [M−H]

### 2-amino-4-methyl-5-nitrobenzoic acid (51-3)

A solution of 2-acetamido-4-methyl-5-nitrobenzoic acid (51-2) (5.6 g, 23.5 mmol) in concentrated HCl (110 mL) was stirred at 100° C. for 4 h. After completion, the mixture was cooled to rt, and cold water (500 mL) was added. The suspension was stirred at rt for an additional 2 h. The product was filtered and washed with water (500 mL), dissolved in acetone (300 mL), dried over Na₂SO₄, filtered, and concentrated to obtain 2-amino-4-methyl-5-nitrobenzoic acid (51-3) (4.5 g, 98%), which was used for the next step without further purification. MS (APCI) m/z: 195.10 [M−H]⁻.

### 2-amino-3-bromo-4-methyl-5-nitrobenzoic acid (51-4)

To a solution of 2-amino-4-methyl-5-nitrobenzoic acid (51-3) (4.8 g, 24.47 mmol) in DMF (95.0 mL) was added NBS (4.79 g, 26.9 mmol) at 0° C. The mixture was stirred at rt for 1 h. After completion, crushed ice was added to the mixture and stirred at rt for 2 h. The product was filtered and washed with water (200 mL), dissolved in acetone (300 mL), dried over Na₂SO₄, filtered, and concentrated to obtain 2-amino-3-bromo-4-methyl-5-nitrobenzoic acid (51-4) (5.2 g, 77%), which was used for the next step without further purification. MS (APCI) m/z: 274.9 [M−H]⁻.

### 2-amino-3-bromo-4-methyl-5-nitrobenzamide (51-5)

To a solution of 2-amino-3-bromo-4-methyl-5-nitrobenzoic acid (51-4) (1.5 g, 5.45 mmol) in DMF (20.0 mL) were added HATU (3.11 g, 8.18 mmol), NH₄Cl (1.75 g, 32.7 mmol), and DIPEA (4.8 mL, 27.3 mmol) at rt. The mixture was stirred at rt for 1 h. After completion, water (100 mL) was added to the mixture and stirred at rt for 1 h. The product was filtered and washed with water (200 mL), dissolved in acetone (200 mL), dried over Na₂SO₄, filtered, and concentrated to obtain 2-amino-3-bromo-4-methyl-5-nitrobenzamide (51-5) (1.48 g, 99%), which was used for the next step without further purification. MS (APCI) m/z: 273.9 [M−H]⁻.

### 8-bromo-7-methyl-6-nitroquinazolin-4 (3H)-one (51-6)

To a solution of 2-amino-3-bromo-4-methyl-5-nitrobenzamide (51-5) (0.4 g, 1.46 mmol) in trimethoxymethane (6.0 mL) was added pTSA (14 mg, 0.073 mmol) at rt. The mixture was stirred at 140° C. for 30 min. After completion, the mixture was concentrated. Pentane (50 mL) was added to the crude mixture and stirred at rt for 1 h. The product was filtered and washed with pentane (60 mL), and dried under high vacuum to obtain 8-bromo-7-methyl-6-nitroquinazolin-4 (3H)-one (51-6) (0.28 g, 68%), which was used for the next step without further purification. MS (APCI) m/z: 283.9 [M−H]⁻.

### 8-bromo-7-methyl-6-nitro-3-((2-(trimethylsilyl)ethoxy)methyl)quinazolin-4 (3H)-one (51-7)

To a solution of 8-bromo-7-methyl-6-nitroquinazolin-4 (3H)-one (51-6) (0.5 g, 1.76 mmol) in DMF (5.5 mL) was added K₂CO₃ (1.23 g, 8.80 mmol) at rt. The mixture was stirred at rt for 1 h followed by slow addition of (2-chloromethoxyethyl)trimethylsilane (1.23 mL, 7.04 mmol). The mixture was stirred at rt for 1 h. After completion, the reaction was quenched with water (20 mL) and extracted with EtOAc (3×50 mL). The combined organic layer was washed with brine (30 mL), dried over Na₂SO₄, filtered, and concentrated. The residue was purified by column chromatography (SiO₂, EtOAc/hexane) to obtain 8-bromo-7-methyl-6-nitro-3-((2-(trimethylsilyl)ethoxy)methyl)quinazolin-4 (3H)-one (51-7) (0.60 g, 82%). MS (APCI) m/z: 415.90 [M+H]⁺.

### 8-(3-methoxy-2,6-dimethylphenyl)-7-methyl-6-nitro-3-((2-(trimethylsilyl)ethoxy)methyl)quinazolin-4 (3H)-one (51-8)

To a micro-wave vial, 8-bromo-7-methyl-6-nitro-3-((2-(trimethylsilyl)ethoxy)methyl)quinazolin-4 (3H)-one (51-7) (0.3 g, 0.724 mmol), (3-methoxy-2,6-dimethylphenyl)boronic acid (INT-5) (261 mg, 1.448 mmol), K₃PO₄ (461 mg, 2.172 mmol), Pd₂(dba)₃ (66 mg, 0.072 mmol), RuPhos (51 mg, 0.109 mmol), and 4 Å molecular sieves were sequentially added. The vial was capped with a rubber septum, evacuated and backfilled with N₂ for three times. Anhydrous degassed toluene (3.6 mL) was added through the septum. The mixture was stirred at rt for 10 min and then at 110° C. for 18 h. After completion, the mixture was concentrated. The residue was purified by column chromatography (SiO₂, EtOAc/hexane) to obtain 8-(3-methoxy-2,6-dimethylphenyl)-7-methyl-6-nitro-3-((2-(trimethylsilyl)ethoxy)methyl)quinazolin-4 (3H)-one (51-8) (0.254 g, 75%). MS (APCI) m/z: 470.10 [M+H]⁺.

### 6-amino-8-(3-methoxy-2,6-dimethylphenyl)-7-methyl-3-((2-(trimethylsilyl)ethoxy)methyl)quinazolin-4 (3H)-one (51-9)

To a solution of 8-(3-methoxy-2,6-dimethylphenyl)-7-methyl-6-nitro-3-((2-(trimethylsilyl)ethoxy)methyl)quinazolin-4 (3H)-one (51-8) (0.25 g, 0.532 mmol) in a mixture of THF (2.0 mL) and IPA (9.5 mL) was added a solution of NH₄Cl (342 mg, 6.39 mmol) in water (4.1 mL). The mixture was stirred at rt for 10 mins. Zinc powder (209 mg, 3.19 mmol) was added in one fraction and the mixture was stirred at 50° C. for 4.0 h. After completion, the mixture was filtered through celite and washed with MeOH (50 mL). Concentrated. DCM (40 mL) was added to the mixture and the product was filtered. The eluent was concentrated to obtain 6-amino-8-(3-methoxy-2,6-dimethylphenyl)-7-methyl-3-((2-(trimethylsilyl)ethoxy)methyl)quinazolin-4 (3H)-one (51-9) (0.197 g, 84%), which was used for the next step without further purification. MS (APCI) m/z: 440.10 [M+H]⁺.

### 6-(dimethylamino)-8-(3-methoxy-2,6-dimethylphenyl)-7-methyl-3-((2-(trimethylsilyl)ethoxy)methyl)quinazolin-4 (3H)-one (51-10)

A mixture of 6-amino-8-(3-methoxy-2,6-dimethylphenyl)-7-methyl-3-((2-(trimethylsilyl)ethoxy)methyl)quinazolin-4 (3H)-one (51-9) (190 mg, 0.432 mmol), 37% aqueous formaldehyde solution (0.6 mL, 21.61 mmol), and 10% Pd/C (38 mg, 50% by wet) in MeOH (3.0 mL) was hydrogenated at 45° C. for 22 h. After completion, the mixture was filtered through celite, washed with MeOH (40 mL). Concentrated. DCM (20 mL) was added and the product was filtered. The eluent was concentrated to obtain 6-(dimethylamino)-8-(3-methoxy-2,6-dimethylphenyl)-7-methyl-3-((2-(trimethylsilyl)ethoxy)methyl)quinazolin-4 (3H)-one (51-10) (0.20 g, 99%), which was used for the next step without further purification. MS (APCI) m/z: 468.20 [M+H]⁺.

### 6-(dimethylamino)-8-(3-methoxy-2,6-dimethylphenyl)-7-methylquinazolin-4 (3H)-one (51-11)

To a solution of 6-(dimethylamino)-8-(3-methoxy-2,6-dimethylphenyl)-7-methyl-3-((2-(trimethylsilyl)ethoxy)methyl)quinazolin-4 (3H)-one (51-10) (0.19 g, 0.406 mmol) in DCM (3.8 mL) was added TFA (1.9 mL) at 0° C. The mixture was stirred at rt for 1 h 15 min. After completion, the reaction was quenched with saturated NaHCO₃ (15 mL) and extracted with DCM (3×50 mL). The combined organic layer was washed with brine (30 mL), dried over Na₂SO₄, filtered, and concentrated to obtain 6-(dimethylamino)-8-(3-methoxy-2,6-dimethylphenyl)-7-methylquinazolin-4 (3H)-one (51-11) (0.136 g, 99%), which was used for the next step without further purification. MS (APCI) m/z: 338.20 [M+H]⁺.

### N⁴-(2,4-dimethoxybenzyl)-8-(3-methoxy-2,6-dimethylphenyl)-N⁶,N⁶,7-trimethylquinazoline-4,6-diamine (51-12)

To a solution of 6-(dimethylamino)-8-(3-methoxy-2,6-dimethylphenyl)-7-methylquinazolin-4 (3H)-one (51-11) (0.130 g, 0.385 mmol) and 2,4-dimethoxyphenyl)methanamine (0.087 mL, 0.578 mmol) in ACN (2.6 mL) were added BOP (0.273 g, 0.616 mmol) and DBU (0.16 mL, 1.156 mmol) at rt. The mixture was stirred at rt for 1 h. After completion, the mixture was concentrated. The residue was purified by column chromatography (SiO₂, EtOAc/hexane) to afford N⁴-(2,4-dimethoxybenzyl)-8-(3-methoxy-2,6-dimethylphenyl)-N⁶,N⁶,7-trimethylquinazoline-4,6-diamine (51-12) (0.18 g, 96%). MS (APCI) m/z: 487.20 [M+H]⁺.

### 8-(3-methoxy-2,6-dimethylphenyl)-N⁶,N⁶, 7-trimethylquinazoline-4,6-diamine (51-13)

A solution of N⁴-(2,4-dimethoxybenzyl)-8-(3-methoxy-2,6-dimethylphenyl)-N⁶,N⁶,7-trimethylquinazoline-4,6-diamine (51-12) (0.12 g, 0.247 mmol) in TFA (3.0 mL) was stirred at 80° C. for 4 h. After completion, the reaction was quenched with saturated NaHCO₃ (30 mL). The mixture was extracted with DCM (3×70 mL). The combined organic layer was dried over Na₂SO₄, filtered, and concentrated to obtain 8-(3-methoxy-2,6-dimethylphenyl)-N⁶,N⁶,7-trimethylquinazoline-4,6-diamine (51-13) (0.082 g, 99%). The crude was used for the next step without further purification. MS (APCI) m/z: 337.10 [M+H]⁺.

### 3-(4-amino-6-(dimethylamino)-7-methylquinazolin-8-yl)-2,4-dimethylphenol (51-A)

To a solution of 8-(3-methoxy-2,6-dimethylphenyl)-N⁶,N⁶,7-trimethylquinazoline-4,6-diamine (51-13) (0.08 g, 0.238 mmol) in DCM (1.5 mL) was added BBr₃ (1M in DCM, 0.71 mL, 0.71 mmol) at 0° C. The mixture was stirred at rt for 6 h. After completion, the reaction was quenched with saturated NaHCO₃ (20 mL) and extracted with DCM (3×90 mL). The combined organic layer was dried over Na₂SO₄, filtered, and concentrated. The residue was purified by column chromatography (SiO₂, MeOH/DCM) to afford 3-(4-amino-6-(dimethylamino)-7-methylquinazolin-8-yl)-2,4-dimethylphenol (51-A) (0.047 g, 61%). MS (APCI) m/z: 323.10 [M+H]⁺.

### Isolation of (R)-3-(4-amino-6-(dimethylamino)-7-methylquinazolin-8-yl)-2,4-dimethylphenol (51-B) and (S)-3-(4-amino-6-(dimethylamino)-7-methylquinazolin-8-yl)-2,4-dimethylphenol (51-C)

The racemic mixture of 3-(4-amino-6-(dimethylamino)-7-methylquinazolin-8-yl)-2,4-dimethylphenol (41 mg) was subjected to chiral SFC purification (LUX-CELLULOSE-4, 30×250 mm column, 30% MeOH with 0.1% 7N NH₃) to afford the title compounds. From Peak 1: (R)-3-(4-amino-6-(dimethylamino)-7-methylquinazolin-8-yl)-2,4-dimethylphenol (51-B) (15.7 mg, 38%). Chiral purity 99.6% (LUX-CELLULOSE-4, 30×250 mm column, 30% MeOH with 0.1% 7N NH₃); ¹H NMR (400 MHz, DMSO-d₆): δ ppm 8.97 (br. s, 1H), 8.09 (s, 1H), 7.72 (s, 1H), 7.53 (bs, 2H), 6.88 (d, J=8.0 Hz, 1H), 6.70 (d, J=8.0 Hz, 1H), 2.73 (s, 6H), 1.95 (s, 3H), 1.59 (s, 3H), 1.54 (s, 3H). MS (ESI) 323.37 [M+H]⁺. From Peak 2: (S)-3-(4-amino-6-(dimethylamino)-7-methylquinazolin-8-yl)-2,4-dimethylphenol (51-C) (15.5 mg, 37%). Chiral purity 97.7% (LUX-CELLULOSE-4, 30×250 mm column, 30% MeOH with 0.1% 7N NH₃); ¹H NMR (400 MHz, DMSO-d₆): δ ppm 8.96 (br. s, 1H), 8.09 (s, 1H), 7.72 (s, 1H), 7.53 (br. s, 2H), 6.88 (d, J=8.0 Hz, 1H), 6.70 (d, J=8.0 Hz, 1H), 2.73 (s, 6H), 1.95 (s, 3H), 1.59 (s, 3H), 1.54 (s, 3H). MS (ESI) 323.37 [M+H]⁺. Compounds 51-B and 51-C are atropisomers, and the shown stereochemistry may be the opposite of that depicted.

### Example 52

### (R)-3-(4-Amino-6,7-dimethylquinazolin-8-yl)-2,4-dimethylphenol (52-B) and (S)-3-(4-amino-6,7-dimethylquinazolin-8-yl)-2,4-dimethylphenol (52-C)

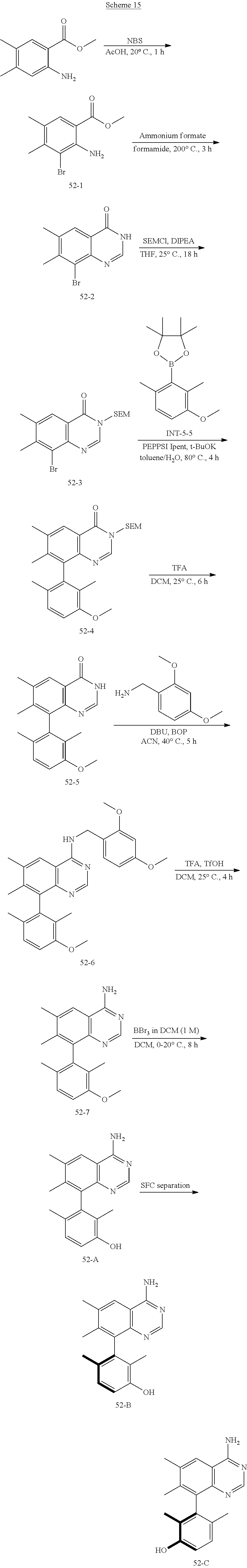

### methyl 2-amino-3-bromo-4,5-dimethylbenzoate (52-1)

To a solution of methyl 2-amino-4,5-dimethylbenzoate (8.0 g, 44.6 mmol) in AcOH (44 mL) was added NBS (8.74 g, 49.1 mmol) in portions at 20° C. The mixture was stirred at 20° C. for 1 h. After completion, the reaction was quenched by water (20 mL) and adjusted the pH to 8.0 with saturated NaHCO₃. The mixture was extracted with EtOAc (3×200 mL). The combined organic layers were washed with brine (200 mL), dried over Na₂SO₄, filtered, and concentrated. The residue was purified by column chromatography (SiO₂, 9% EtOAc/PET) to give methyl 2-amino-3-bromo-4,5-dimethylbenzoate (52-1) (9.20 g, 79%). ¹H NMR (400 MHz, DMSO-d₆): δ ppm 7.57 (s, 1H), 6.56 (br s, 2H), 3.80 (s, 3H), 2.31 (s, 3H), 2.18 (s, 3H). MS (ESI) m/z: 258.0 [M+H]⁺.

### 8-bromo-6,7-dimethylquinazolin-4 (3H)-one (52-2)

A mixture of methyl 2-amino-3-bromo-4,5-dimethylbenzoate (52-1) (1.00 g, 3.87 mmol) and ammonium formate (488 mg, 7.75 mmol) in formamide (3.5 mL) was stirred at 200° C. for 3 h. 9 Additional batches of 1 g each were set up. After completion, all ten reaction mixtures were combined. The combined mixture was cooled to 20° C., diluted with water (200 mL). The suspension was sonicated for 30 min. The product was filtered. The filter cake was washed with water (200 mL) and PET (200 mL). The product was dried to give 8-bromo-6,7-dimethylquinazolin-4 (3H)-one (52-2) (7.45 g, 76%). ¹H NMR (400 MHz, DMSO-d₆): δ ppm 12.34 (br s, 1H), 8.14 (s, 1H), 7.89 (s, 1H), 2.51 (s, 3H), 2.44 (s, 3H). MS (ESI) m/z: 253.0 [M+H]⁺.

### 8-bromo-6,7-dimethyl-3-((2-(trimethylsilyl)ethoxy)methyl)quinazolin-4 (3H)-one (52-3)

To a solution of 8-bromo-6,7-dimethylquinazolin-4 (3H)-one (52-2) (7.0 g, 27.6 mmol) in THF (70 mL) were added DIPEA (21.4 g, 165 mmol) and SEM-Cl (11.5 g, 69.1 mmol) at 0° C. After addition, the mixture was stirred at 25° C. for 18 h. After completion, the reaction was quenched with water (100 mL), extracted with EtOAc (3×100 mL). The combined organic layers were dried over Na₂SO₄, filtered, and concentrated. The residue was purified by column chromatography (SiO₂, 35% EtOAc/PET) to give 8-bromo-6,7-dimethyl-3-((2-(trimethylsilyl)ethoxy)methyl)quinazolin-4 (3H)-one (52-3) (6.84 g, 64%). ¹H NMR (400 MHz, DMSO-d₆): δ ppm 8.47 (s, 1H), 7.97 (s, 1H), 5.36 (s, 2H), 3.62 (t, J=7.6 Hz, 2H), 2.53 (s, 3H), 2.46 (s, 3H), 0.94-0.83 (m, 2H), −0.03-−0.15 (m, 9H). MS (ESI) m/z: 383.2 [M+H]⁺.

### 8-(3-methoxy-2,6-dimethylphenyl)-6,7-dimethyl-3-((2-(trimethylsilyl)ethoxy)methyl)quinazolin-4 (3H)-one (52-4)

To a solution of 8-bromo-6,7-dimethyl-3-((2-(trimethylsilyl)ethoxy)methyl)quinazolin-4 (3H)-one (52-3) (500 mg, 1.30 mmol) and 2-(3-methoxy-2,6-dimethylphenyl)-4,4,5,5-tetramethyl-1,3,2-dioxaborolane (410 mg, 1.57 mmol) in a mixture of H₂O (1.0 mL), t-BuOH (4.0 mL) and toluene (4.0 mL) were added t-BuOK (292 mg, 2.61 mmol) and PEPPSI Ipent (103 mg, 0.13 mmol) at rt. The mixture was stirred at 80° C. for 4 h. Additional three equal batch of reactions were set up as described above. All four reaction mixtures were combined. The combined reaction mixture was diluted with EtOAc (50 mL) and washed with brine (20 mL). The organic layer was dried over Na₂SO₄, filtered, and concentrated. The residue was purified by prep-HPLC (C18, ACN/H₂O with 0.075% TFA) to give 8-(3-methoxy-2,6-dimethylphenyl)-6,7-dimethyl-3-((2-(trimethylsilyl)ethoxy)methyl)quinazolin-4 (3H)-one (52-4) (380 mg, 16%) as a light yellow gel. ¹H NMR (400 MHz, CDCl₃): δ ppm 8.24-8.09 (m, 2H), 7.12 (d, J=8.4 Hz, 1H), 6.85 (d, J=8.4 Hz, 1H), 5.42 (s, 2H), 3.86 (s, 3H), 3.76-3.65 (m, 2H), 2.49 (s, 3H), 2.01 (s, 3H), 1.76 (d, J=10.8 Hz, 6H), 1.00-0.92 (m, 2H), −0.01 (s, 9H). MS (ESI) m/z: 439.3 [M+H]⁺.

### 8-(3-methoxy-2,6-dimethylphenyl)-6,7-dimethylquinazolin-4 (3H)-one (52-5)

To a solution of 8-(3-methoxy-2,6-dimethylphenyl)-6,7-dimethyl-3-((2-(trimethylsilyl)ethoxy)methyl)quinazolin-4 (3H)-one (52-4) (380 mg, 0.86 mmol) in DCM (3.8 mL) was added TFA (3.8 mL) dropwise at 25° C. The mixture was stirred at 25° C. for 6 h. After completion, the mixture was concentrated. The residue was purified by column chromatography (SiO₂, 15% EtOAC/PET) to give 8-(3-methoxy-2,6-dimethylphenyl)-6,7-dimethylquinazolin-4 (3H)-one (52-5) (260 mg, 92%). ¹H NMR (400 MHz, CDCl₃): δ ppm 8.98 (s, 1H), 8.18 (s, 1H), 7.17 (d, J=8.4 Hz, 1H), 6.91 (d, J=8.4 Hz, 1H), 3.86 (s, 3H), 2.54 (s, 3H), 2.09 (s, 3H), 1.75 (d, J=14.0 Hz, 6H). MS (ESI) m/z: 309.2 [M+H]⁺.

### N-(2,4-dimethoxybenzyl)-8-(3-methoxy-2,6-dimethylphenyl)-6,7-dimethylquinazolin-4-amine (52-6)

To a solution of 8-(3-methoxy-2,6-dimethylphenyl)-6,7-dimethylquinazolin-4 (3H)-one (52-5) (190 mg, 0.61 mmol), DBU (187 mg, 1.23 mmol) and BOP (545 mg, 1.23 mmol) in ACN (1.9 mL) was added DMBNH₂ (309 mg, 1.85 mmol) at 25° C. The mixture was stirred at 40° C. for 5 h. After completion, the mixture was diluted with EtOAc (50 mL), washed with brine (20 mL). The organic layer was dried over Na₂SO₄, filtered, and concentrated. The residue was purified by column chromatography (SiO₂, 50% EtOAc/PET) to give N-(2,4-dimethoxybenzyl)-8-(3-methoxy-2,6-dimethylphenyl)-6,7-dimethylquinazolin-4-amine (52-6) (396 mg, 84% yield). ¹H NMR (400 MHz, DMSO-d₆): δ ppm 8.33 (br s, 2H), 7.26 (d, J=8.4 Hz, 2H), 6.57 (d, J=2.4 Hz, 1H), 6.55 (d, J=2.4 Hz, 1H), 6.49 (dd, J=8.4, 2.0 Hz, 1H), 4.74 (br s, 2H), 3.78 (s, 6H), 3.75 (s, 3H), 2.47 (s, 3H), 1.97 (s, 3H), 1.68 (s, 3H), 1.62 (s, 3H). MS (ESI) m/z: 458.3 [M+H]⁺.

### 8-(3-methoxy-2,6-dimethylphenyl)-6,7-dimethylquinazolin-4-amine (52-7)

To a solution of N-(2,4-dimethoxybenzyl)-8-(3-methoxy-2,6-dimethylphenyl)-6,7-dimethylquinazolin-4-amine (52-6) (400 mg, 0.52 mmol) in DCM (4.0 mL) were added TFA (1.20 g, 10.4 mmol) and TfOH (787 mg, 5.25 mmol) at 25° C. The mixture was stirred at 25° C. for 4 h. After completion, the reaction was quenched with saturated NaHCO₃ (30 mL) to adjust the pH ~7. The mixture was extracted with EtOAc (3×20 mL). The combined organic layers were washed with brine (10 mL), dried over Na₂SO₄, filtered, and concentrated. The residue was purified by column chromatography (SiO₂, 80% EtOAc/PET) to give 8-(3-methoxy-2,6-dimethylphenyl)-6,7-dimethylquinazolin-4-amine (52-7) (120 mg, 70%). ¹H NMR (400 MHz, DMSO-d₆): δ ppm 8.13 (s, 1H), 8.03 (s, 1H), 7.55 (br d, J=2.4 Hz, 2H), 7.07 (d, J=8.4 Hz, 1H), 6.85 (d, J=8.4 Hz, 1H), 3.80 (s, 3H), 2.43 (s, 3H), 1.93 (s, 3H), 1.64 (s, 3H), 1.56 (s, 3H). MS (ESI) m/z: 308.2 [M+H]⁺.

### 3-(4-amino-6,7-dimethylquinazolin-8-yl)-2,4-dimethylphenol (52-A)

To a solution of 8-(3-methoxy-2,6-dimethylphenyl)-6,7-dimethylquinazolin-4-amine (52-7) (150 mg, 0.48 mmol) in DCM (3.0 mL) was added BBr₃ (1M in DCM, 0.97 mL, 0.97 mmol) dropwise at 0° C. The mixture was stirred at 20° C. for 8 h. After completion, the reaction was quenched with saturated NaHCO₃ (3.0 mL) to adjust the pH ~7. The mixture was extracted with EtOAc (3×20 mL). The combined organic layers were washed with brine (10 mL), dried over Na₂SO₄, filtered, and concentrated. The residue was triturated with DCM (1 mL). The product was filtered and washed with MTBE (10 mL). The product was dried to give 3-(4-amino-6,7-dimethylquinazolin-8-yl)-2,4-dimethylphenol (52-A) (90.0 mg, 61%). ¹H NMR (400 MHz, DMSO-d₆): δ ppm 8.98 (s, 1H), 8.14 (s, 1H), 8.03 (s, 1H), 7.56 (br s, 2H), 6.89 (d, J=8.0 Hz, 1H), 6.71 (d, J=8.0 Hz, 1H), 2.43 (s, 3H), 1.94 (s, 3H), 1.59 (s, 3H), 1.53 (s, 3H). MS (ESI) m/z: 294.3 [M+H]⁺.

### Isolation of (R)-3-(4-amino-6,7-dimethylquinazolin-8-yl)-2,4-dimethylphenol (52-B) and (S)-3-(4-amino-6,7-dimethylquinazolin-8-yl)-2,4-dimethylphenol (52-C)

The racemic mixture of 3-(4-amino-6,7-dimethylquinazolin-8-yl)-2,4-dimethylphenol (52-A) (70 mg) was subjected to chiral SFC purification (DAICEL CHIRALCEL OJ, 30×250 mm column, CO₂/25% MeOH with 0.1% NH₄OH) to afford the title compounds. From Peak 1: (R)-3-(4-amino-6,7-dimethylquinazolin-8-yl)-2,4-dimethylphenol (52-B) (15.30 mg, 21%). Chiral purity 99.24% (CHIRALCEL OJ-3, 4.6×150 mm column, CO₂/10% MeOH with 0.5% isopropylamine); ¹H NMR (400 MHz, DMSO-d₆): δ ppm 8.91 (br s, 1H), 8.13 (s, 1H), 8.01 (s, 1H), 7.52 (br s, 2H), 6.88 (d, J=8.0 Hz, 1H), 6.71 (d, J=8.0 Hz, 1H), 2.42 (s, 3H), 1.94 (s, 3H), 1.59 (s, 3H), 1.53 (s, 3H). MS (ESI) m/z: 294.2 [M+H]⁺. From Peak 2: (S)-3-(4-amino-6,7-dimethylquinazolin-8-yl)-2,4-dimethylphenol (52-C) (14.75 mg, 20%). Chiral purity 93.46% (CHIRAlCEL OJ-3, 4.6×150 mm column, CO₂/10% MeOH with 0.5% isopropylamine); ¹H NMR (400 MHz, DMSO-d₆): δ ppm 8.98 (br s, 1H), 8.13 (s, 1H), 8.01 (s, 1H), 7.53 (br s, 2H), 6.88 (d, J=8.4 Hz, 1H), 6.70 (d, J=8.0 Hz, 1H), 2.42 (s, 3H), 1.94 (s, 3H), 1.58 (s, 3H), 1.53 (s, 3H). MS (ESI) m/z: 294.3 [M+H]⁺. Compounds 52-B and 52-C are atropisomers, and the shown stereochemistry may be the opposite of that depicted.

### Example 53

### (R)-3-(4-Amino-6-methylquinazolin-8-yl)-2,4-dimethylphenol (53-B) and (S)-3-(4-amino-6-methylquinazolin-8-yl)-2,4-dimethylphenol (53-C)

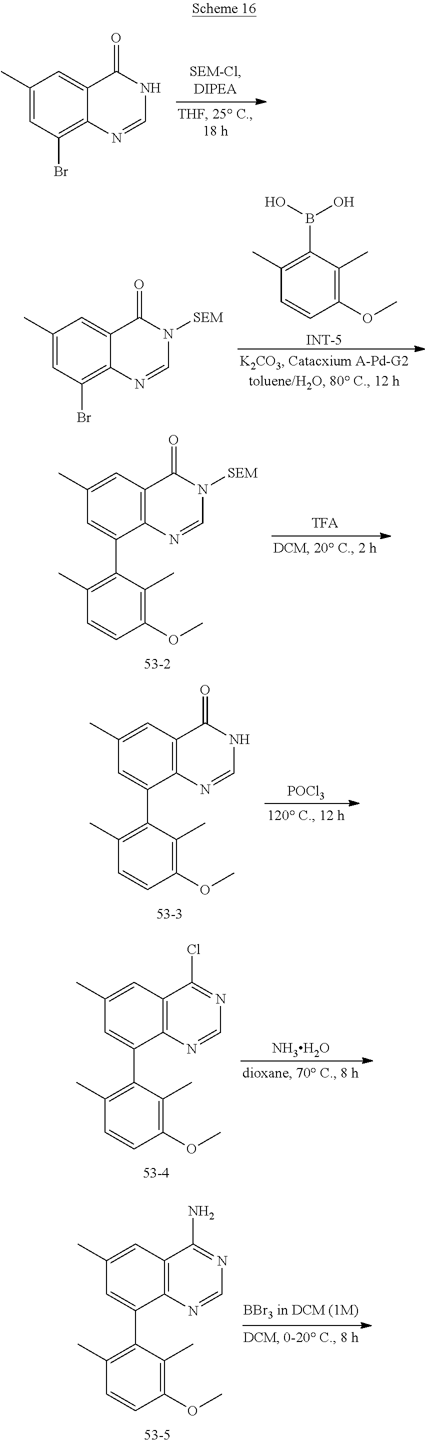

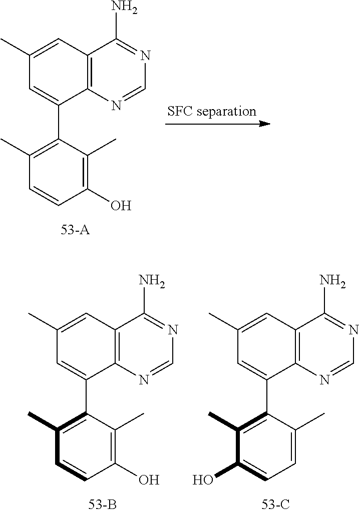

### 8-bromo-6-methyl-3-((2-(trimethylsilyl)ethoxy)methyl)quinazolin-4 (3H)-one (53-1)

To a solution of 8-bromo-6-methylquinazolin-4 (3H)-one (3.50 g, 14.6 mmol) in THF (35 mL) were added DIPEA (11.3 g, 87.8 mmol) and SEM-Cl (6.10 g, 36.6 mmol) at 0° C. After addition, the mixture was stirred at 25° C. for 18 h. After completion, the reaction was quenched with water (50 mL), extracted with EtOAc (3×100 mL). The combined organic layers were dried over Na₂SO₄, filtered, and concentrated. The residue was purified by column chromatography (SiO₂, 35% EtOAc/PET) to give 8-bromo-6-methyl-3-((2-(trimethylsilyl)ethoxy)methyl)quinazolin-4 (3H)-one (53-1) (5.01 g, 92%). ¹H NMR (400 MHz, DMSO-d₆): δ ppm 8.48 (s, 1H), 8.08-7.93 (m, 2H), 5.45-5.26 (m, 2H), 3.67-3.57 (m, 2H), 2.45 (s, 3H), 0.91-0.84 (m, 2H), −0.07 (s, 9H). MS (ESI) m/z: 371.1 [M+H]⁺.

### 8-(3-methoxy-2,6-dimethylphenyl)-6-methyl-3-((2-(trimethylsilyl)ethoxy)methyl)quinazolin-4 (3H)-one (53-2)

To a solution of (3-methoxy-2,6-dimethylphenyl)boronic acid (INT-5) (350 mg, 1.95 mmol) and 8-bromo-6-methyl-3-((2-(trimethylsilyl)ethoxy)methyl)quinazolin-4 (3H)-one (53-1) (600 mg, 1.62 mmol) in a mixture of toluene (6 mL) and H₂O (1.5 mL) were added Catacxium A-Pd-G₂ (108 mg, 0.16 mmol) and K₂CO₃ (673 mg, 4.87 mmol). The mixture was purged with N₂ for three times and stirred at 80° C. for 12 h. After completion, the reaction was quenched with water (5 mL) and extracted with EtOAc (3×10 mL). The combined organic layers were washed with brine (10 mL), dried over Na₂SO₄, filtered, and concentrated. The residue was purified by column chromatography (SiO₂, 30% EtOAc/PET) to give 8-(3-methoxy-2,6-dimethylphenyl)-6-methyl-3-((2-(trimethylsilyl)ethoxy)methyl)quinazolin-4 (3H)-one (53-2) (580 mg, 84%). ¹H NMR (400 MHz, DMSO-d₆): δ ppm 8.39 (s, 1H), 8.17 (s, 1H), 7.48 (d, J=2.0 Hz, 1H), 7.14 (d, J=8.4 Hz, 1H), 6.96 (d, J=8.4 Hz, 1H), 5.42 (s, 2H), 3.87 (s, 3H), 3.69 (t, J=8.0 Hz, 2H), 3.41-3.37 (m, 3H), 1.85 (s, 3H), 1.76 (s, 3H), 0.97-0.89 (m, 2H), 0.08-−0.05 (m, 9H). MS (ESI) m/z: 425.3 [M+H]⁺.

### 8-(3-methoxy-2,6-dimethylphenyl)-6-methylquinazolin-4 (3H)-one (53-3)

To a solution of 8-(3-methoxy-2,6-dimethylphenyl)-6-methyl-3-((2-(trimethylsilyl)ethoxy)methyl)quinazolin-4 (3H)-one (53-2) (500 mg, 1.18 mmol) in DCM (5 mL) was added TFA (5 mL) at 20° C. The mixture was stirred at this temperature for 2 h. After completion, the reaction was quenched with water (3 mL) and extracted with DCM (3×5 mL). The combined organic layers were dried over Na₂SO₄, filtered, and concentrated. The residue was purified by column chromatography (SiO₂, 5% MeOH/DCM) to give 8-(3-methoxy-2,6-dimethylphenyl)-6-methylquinazolin-4 (3H)-one (53-3) (340 mg, 98%). ¹H NMR (400 MHz, DMSO-d₆): δ ppm 7.97 (d, J=1.2 Hz, 1H), 7.91 (s, 1H), 7.42-7.32 (m, 1H), 7.12-7.02 (m, 1H), 6.92-6.83 (m, 1H), 3.82-3.77 (m, 3H), 2.47 (s, 3H), 1.78 (s, 3H), 1.69 (s, 3H). MS (ESI) m/z: 295.2 [M+H]⁺.

### 4-chloro-8-(3-methoxy-2,6-dimethylphenyl)-6-methylquinazoline (53-4)

A solution of 8-(3-methoxy-2,6-dimethylphenyl)-6-methylquinazolin-4 (3H)-one (53-3) (380 mg, 1.29 mmol) in POCl₃ (11.4 mL) was stirred at 120° C. for 12 h. After completion, the mixture was cooled to rt and concentrated under N₂ blowing. The residue was triturated with MTBE (1 mL). The product was filtered and dried to give 4-chloro-8-(3-methoxy-2,6-dimethylphenyl)-6-methylquinazoline (53-4) (400 mg, 99%). The crude product was used to the next step without further purification. MS (ESI) m/z: 313.1 [M+H]⁺.

### 8-(3-methoxy-2,6-dimethylphenyl)-6-methylquinazolin-4-amine (53-5)

To a solution of 4-chloro-8-(3-methoxy-2,6-dimethylphenyl)-6-methylquinazoline (53-4) (400 mg, 1.28 mmol) in dioxane (1 mL) was added NH₃ in H₂O (30 wt %, 4.48 g, 38.3 mmol) dropwise at 20° C. The mixture was stirred at 70° C. for 8 h. After completion, the mixture was diluted with H₂O (20 mL) and extracted with EtOAc (3×20 mL). The combined organic layers were washed with brine (10 mL), dried over Na₂SO₄, filtered, and concentrated. The residue was purified by column chromatography (SiO₂, 60% EtOAc/PET) to give 8-(3-methoxy-2,6-dimethylphenyl)-6-methylquinazolin-4-amine (53-5) (180 mg, 46%). ¹H NMR (400 MHz, DMSO-d₆): δ ppm 8.18 (s, 1H), 8.04 (s, 1H), 7.65 (br s, 2H), 7.31 (d, J=1.6 Hz, 1H), 7.04 (d, J=8.4 Hz, 1H), 6.86 (d, J=8.4 Hz, 1H), 3.79 (s, 3H), 2.47 (s, 3H), 1.74 (s, 3H), 1.65 (s, 3H). MS (ESI) m/z: 294.3 [M+H]⁺.

### 3-(4-amino-6-methylquinazolin-8-yl)-2,4-dimethylphenol (53-A)

To a solution of 8-(3-methoxy-2,6-dimethylphenyl)-6-methylquinazolin-4-amine (53-5) (170 mg, 0.58 mmol) in DCM (3.4 mL) was added BBr₃ (1M in DCM, 1.16 mL, 1.16 mmol) dropwise at 0° C. The mixture was stirred at 20° C. for 8 h. After completion, the reaction was quenched with saturated NaHCO₃ (20 mL) to adjust pH ~7. The mixture was extracted with EtOAc (3×20 mL). The combined organic layers were washed with brine (10 mL), dried over Na₂SO₄, filtered, and concentrated to give racemic 3-(4-amino-6-methylquinazolin-8-yl)-2,4-dimethylphenol (53-A) (160 mg, 98%). ¹H NMR (400 MHz, DMSO-d₆): δ ppm 8.99 (s, 1H), 8.18 (s, 1H), 8.02 (d, J=0.8 Hz, 1H), 7.63 (br s, 2H), 7.30 (d, J=1.6 Hz, 1H), 6.86 (d, J=8.4 Hz, 1H), 6.71 (d, J=8.0 Hz, 1H), 2.47 (s, 3H), 1.69 (s, 3H), 1.62 (s, 3H). MS (ESI) m/z: 280.0 [M+H]⁺.

### Isolation of (R)-3-(4-amino-6-methylquinazolin-8-yl)-2,4-dimethylphenol (53-B) and (S)-3-(4-amino-6-methylquinazolin-8-yl)-2,4-dimethylphenol (53-C)

The racemic mixture of 3-(4-amino-6-methylquinazolin-8-yl)-2,4-dimethylphenol (53-A) (160 mg) was subjected to chiral SFC purification (DAICEL CHIRALCEL OJ, 30×250 mm column, CO₂/20% EtOH with 0.1% NH₄OH) to afford the title compounds. From Peak 1: (R)-3-(4-amino-6-methylquinazolin-8-yl)-2,4-dimethylphenol (53-B) (50.76 mg, 31%). Chiral purity 100% [CHIRAlCEL OJ-3, 4.6×150 mm column, CO₂/10% EtOH with 0.2% NH₃ in MeOH (7M)]; ¹H NMR (400 MHz, DMSO-d₆): δ ppm 8.99 (s, 1H), 8.18 (s, 1H), 8.02 (d, J=0.8 Hz, 1H), 7.63 (br s, 2H), 7.30 (d, J=1.6 Hz, 1H), 6.86 (d, J=8.4 Hz, 1H), 6.71 (d, J=8.0 Hz, 1H), 2.47 (s, 3H), 1.69 (s, 3H), 1.62 (s, 3H). MS (ESI) m/z: 280.2 [M+H]⁺. From Peak 2: (S)-3-(4-amino-6-methylquinazolin-8-yl)-2,4-dimethylphenol (53-C) (54.51 mg, 34%). Chiral purity 99.30% [CHIRAlCEL OJ-3, 4.6×150 mm column, CO₂/10% EtOH with 0.2% NH₃ in MeOH (7M)]; ¹H NMR (400 MHz, DMSO-d₆): δ ppm 8.99 (s, 1H), 8.18 (s, 1H), 8.02 (d, J=0.8 Hz, 1H), 7.63 (br s, 2H), 7.30 (d, J=1.6 Hz, 1H), 6.86 (d, J=8.4 Hz, 1H), 6.71 (d, J=8.0 Hz, 1H), 2.47 (s, 3H), 1.69 (s, 3H), 1.62 (s, 3H). MS (ESI) m/z: 280.3 [M+H]⁺. Compounds 53-B and 53-C are atropisomers, and the shown stereochemistry may be the opposite of that depicted.

### Example 54

### 3-((S)-4-amino-6-((R)-3-fluoro-3-methylpyrrolidin-1-yl)pyrido[3,4-d]pyrimidin-8-yl)-2,4-dimethylphenol (54-B) and 3-((R)-4-amino-6-((R)-3-fluoro-3-methylpyrrolidin-1-yl)pyrido[3,4-d]pyrimidin-8-yl)-2,4-dimethylphenol (54-C)

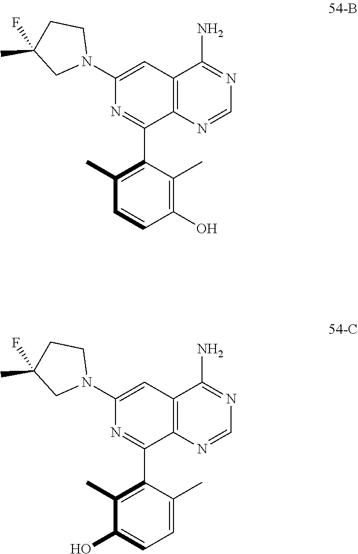

The racemic mixture of 3-(4-amino-6-((R)-3-fluoro-3-methylpyrrolidin-1-yl)pyrido[3,4-d]pyrimidin-8-yl)-2,4-dimethylphenol (100 mg) was prepared according to the procedure described in Example 38 and was subjected to chiral SFC purification (CHIRALCEL OJ-H, 30×250 mm column, CO₂/77% IPA/ACN (1:1) with 0.1% isopropylamine) to afford the title compounds. From Peak 1: 3-((S)-4-amino-6-((R)-3-fluoro-3-methylpyrrolidin-1-yl) pyrido[3,4-d]pyrimidin-8-yl)-2,4-dimethylphenol (54-B) (28.3 mg, 28%). Chiral purity 98.56% (CHIRALCEL OJ-H, 4.6×250 mm column, CO₂/25% IPA/ACN (1:1); ¹H NMR (400 MHz, DMSO-d₆): δ ppm 9.02 (s, 1H), 8.02 (s, 1H), 7.72 (brs, 2H), 6.92 (s, 1H), 6.85 (d, J=8.0 Hz, 1H), 6.73 (d, J=8.0 Hz, 1H), 3.73-3.64 (m, 2H), 3.55-3.43 (m, 2H), 2.28-2.10 (m, 2H), 1.72 (s, 3H), 1.63 (s, 3H), 1.58 (d, J=20.8 Hz, 3H); MS (ESI) m/z: 368.29 [M+H]⁺. From Peak 2: 3-((R)-4-amino-6-((R)-3-fluoro-3-methylpyrrolidin-1-yl) pyrido[3,4-d]pyrimidin-8-yl)-2,4-dimethylphenol (54-C) (23.2 mg, 23%). Chiral purity 98.64% (CHIRALCEL OJ-H, 4.6×250 mm column, CO₂/25% IPA/ACN (1:1); ¹H NMR (400 MHz, DMSO-d₆): δ ppm 9.03 (s, 1H), 8.02 (s, 1H), 7.72 (brs, 2H), 6.92 (s, 1H), 6.85 (d, J=8.0 Hz, 1H), 6.73 (d, J=8.4 Hz, 1H), 3.76-3.64 (m, 2H), 3.56-3.44 (m, 2H), 2.28-2.07 (m, 2H), 1.72 (s, 3H), 1.63 (s, 3H), 1.58 (d, J=20.8 Hz, 3H); MS (ESI) m/z: 368.29 [M+H]⁺. Compounds 54-B (Peak 1) and 54-C (Peak 2) are atropisomers, and the shown stereochemistry may be the opposite of that depicted.

### Example 55

### 3-((S)-4-amino-6-((S)-3-fluoro-3-methylpyrrolidin-1-yl)pyrido[3,4-d]pyrimidin-8-yl)-2,4-dimethylphenol (55-B) and 3-((R)-4-amino-6-((S)-3-fluoro-3-methylpyrrolidin-1-yl)pyrido[3,4-d]pyrimidin-8-yl)-2,4-dimethylphenol (55-C)

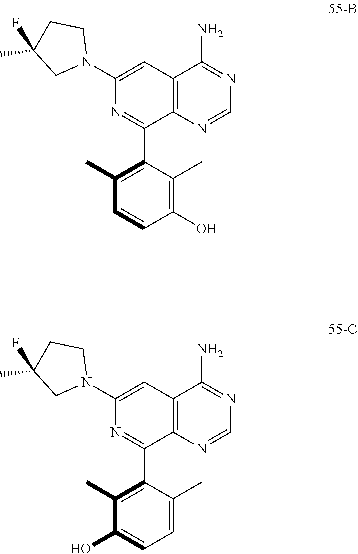

The racemic mixture of 3-(4-amino-6-((S)-3-fluoro-3-methylpyrrolidin-1-yl)pyrido[3,4-d]pyrimidin-8-yl)-2,4-dimethylphenol (59 mg) was prepared according to the procedure described in Example 38 and was subjected to chiral SFC purification [CHIRALCEL OJ-H, 4.6×250 mm column, CO₂/20% IPA/ACN (2:8) with 0.2% isopropylamine] to afford the title compounds. From Peak 1: 3-((S)-4-amino-6-((S)-3-fluoro-3-methylpyrrolidin-1-yl) pyrido[3,4-d]pyrimidin-8-yl)-2,4-dimethylphenol (55-B) (21 mg, 8%). Chiral purity 99.85% [CHIRALCEL OJ-H, 4.6×250 mm column, CO₂/25% IPA/ACN (1:1) with 0.2% diethylamine]; ¹H NMR (400 MHz, DMSO-d₆): δ ppm 9.00 (s, 1H), 8.03 (s, 1H), 7.73 (br s, 2H), 6.93 (s, 1H), 6.85 (d, J=8.4 Hz, 1H), 6.73 (d, J=8.0 Hz, 1H), 3.80-3.60 (m, 2H), 3.60-3.41 (m, 2H), 2.30-2.10 (m, 2H), 1.72 (s, 3H), 1.63 (s, 3H), 1.58 (d, J=20.8 Hz, 3H); MS (ESI) m/z: 368.27 [M+H]⁺. From Peak 2: 3-((R)-4-amino-6-((S)-3-fluoro-3-methylpyrrolidin-1-yl) pyrido[3,4-d]pyrimidin-8-yl)-2,4-dimethylphenol (55-C) (19.5 mg, 7%). Chiral purity 99.35% [CHIRALCEL OJ-H, 4.6×250 mm column, CO₂/25% IPA/ACN (1:1) with 0.2% diethylamine]; ¹H NMR (400 MHz, DMSO-d₆): δ ppm 9.03 (s, 1H), 8.03 (s, 1H), 7.73 (br s, 2H), 6.93 (s, 1H), 6.85 (d, J=8.0 Hz, 1H), 6.73 (d, J=8.0 Hz, 1H), 3.82-3.60 (m, 2H), 3.59-3.40 (m, 2H), 2.31-2.10 (m, 2H), 1.73 (s, 3H), 1.63 (s, 3H), 1.58 (d, J=20.8 Hz, 3H); MS (ESI) m/z: 368.27 [M+H]⁺. Compounds 55-B (Peak 1) and 55-C (Peak 2) are atropisomers, and the shown stereochemistry may be the opposite of that depicted.

### Example 56

### (S)-3-(4-amino-6-(3,6-dihydro-2H-pyran-4-yl)pyrido[3,4-d]pyrimidin-8-yl)-2,4-dimethylphenol (56-B) and (R)-3-(4-amino-6-(3,6-dihydro-2H-pyran-4-yl)pyrido[3,4-d]pyrimidin-8-yl)-2,4-dimethylphenol (56-C)

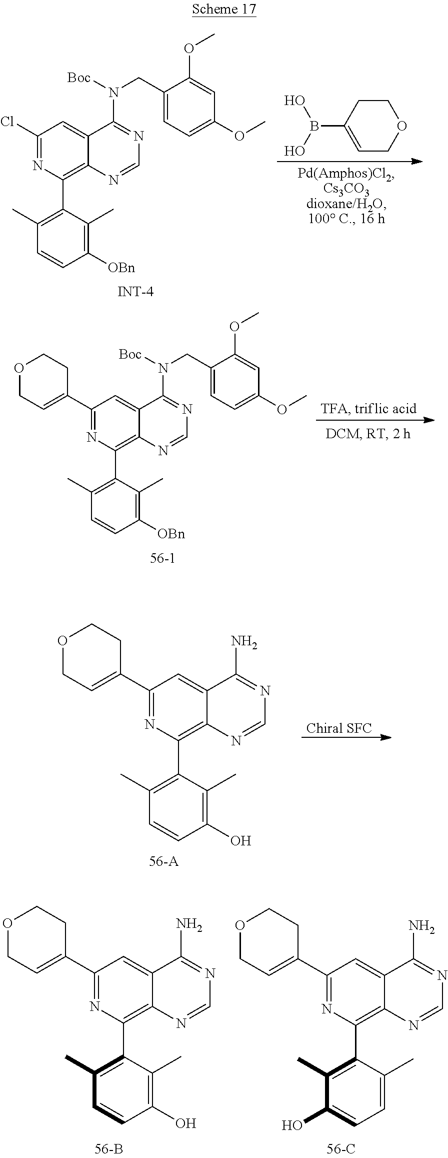

### tert-butyl (8-(3-(benzyloxy)-2,6-dimethylphenyl)-6-(3,6-dihydro-2H-pyran-4-yl)pyrido[3,4-d]pyrimidin-4-yl)(2,4-dimethoxybenzyl)carbamate (56-1)

To a solution of tert-butyl (8-(3-(benzyloxy)-2,6-dimethylphenyl)-6-chloropyrido[3,4-d]pyrimidin-4-yl)(2,4-dimethoxybenzyl)carbamate (INT-4) (300 mg, 0.468 mmol) and (3,6-dihydro-2H-pyran-4-yl) boronic acid (71.83 mg, 0.561 mmol) in 1,4-dioxane (4.8 mL) and water (1.2 mL) was added Cs₂CO₃ (457.4 mg, 1.404 mmol) at rt. The mixture was purged with N₂ for 10 min. To this mixture, Pd(Amphos)Cl₂ (49.69 mg, 0.070 mmol) was added, and the reaction mixture was stirred at 100° C. for 16 h. After completion, the reaction mixture was carefully quenched with water (20 mL) and extracted with EtOAc (3×10 mL). The combined organic layer was washed with brine (10 mL), dried over Na₂SO₄, filtered, and concentrated. The residue was purified by column chromatography (SiO₂, EtOAc/pet ether) to afford tert-butyl (8-(3-(benzyloxy)-2,6-dimethylphenyl)-6-(3,6-dihydro-2H-pyran-4-yl) pyrido[3,4-d]pyrimidin-4-yl)(2,4-dimethoxybenzyl)carbamate (56-1) (170 mg, 53%). MS (ESI) m/z: 690.5 [M+H]⁺.

### 3-(4-amino-6-(3,6-dihydro-2H-pyran-4-yl)pyrido[3,4-d]pyrimidin-8-yl)-2,4-dimethylphenol (56-A)

To a solution of tert-butyl (8-(3-(benzyloxy)-2,6-dimethylphenyl)-6-(3,6-dihydro-2H-pyran-4-yl)pyrido[3,4-d]pyrimidin (3-1) (170 mg, 0.247 mmol) in DCM (1.7 mL) were added TFA (0.38 mL, 4.94 mmol) and triflic acid (0.22 mL, 2.47 mmol) at rt. The reaction mixture was stirred at rt for 2 h. After completion, the reaction mixture was concentrated, carefully quenched with saturated NaHCO₃ (50 mL) and extracted with EtOAc (2×50 mL). The combined organic layer was washed with brine (60 mL), dried over Na₂SO₄, filtered, and concentrated. The residue was purified by column chromatography (SiO₂, MeOH/DCM) to afford 3-(4-amino-6-(3,6-dihydro-2H-pyran-4-yl) pyrido[3,4-d]pyrimidin-8-yl)-2,4-dimethylphenol (56-A) (85 mg, 98% yield). MS (ESI) m/z: 349.3 [M+H]⁺.

### Isolation of (S)-3-(4-amino-6-(3,6-dihydro-2H-pyran-4-yl)pyrido[3,4-d]pyrimidin-8-yl)-2,4-dimethylphenol (56-B) and (R)-3-(4-amino-6-(3,6-dihydro-2H-pyran-4-yl)pyrido[3,4-d]pyrimidin-8-yl)-2,4-dimethylphenol (56-C)

The racemic mixture of 3-(4-amino-6-(3,6-dihydro-2H-pyran-4-yl) pyrido[3,4-d]pyrimidin-8-yl)-2,4-dimethylphenol (56-A) (90 mg) was subjected to chiral SFC purification [CHIRALPAK IH, 30×250 mm column, CO₂/27% IPA/ACN (1:1) with 0.1% isopropylamine] to afford the title compounds. From Peak 1: (S)-3-(4-amino-6-(3,6-dihydro-2H-pyran-4-yl)pyrido[3,4-d]pyrimidin-8-yl)-2,4-dimethylphenol (56-B) (11.5 mg, 12.78%). Chiral purity 95.29% [CHIRALPAK IH, 4.6×250 mm column, CO₂/30% IPA/ACN (1:1) with 0.2% isopropylamine]; ¹H NMR (400 MHz, DMSO-d₆): δ ppm 9.07 (s, 1H), 8.31 (s, 1H), 8.25-8.06 (m, 3H), 6.89-6.86 (m, 2H), 6.75 (d, J=8.0 Hz, 1H), 4.29 (d, J=2.4 Hz, 2H), 3.87 (t, J=5.2 Hz, 2H), 2.60 (brs, 2H), 1.69 (s, 3H), 1.61 (s, 3H); MS (ESI) m/z: 349.36 [M+H]⁺. From Peak 2: (R)-3-(4-amino-6-(3,6-dihydro-2H-pyran-4-yl)pyrido[3,4-d]pyrimidin-8-yl)-2,4-dimethylphenol (56-C) (16.9 mg, 18.78%). Chiral purity 85.59% [CHIRALPAK IH, 4.6×250 mm column, CO₂/30% IPA/ACN (1:1) with 0.2% isopropylamine]; ¹H NMR (400 MHz, DMSO-d₆): δ ppm 8.99 (s, 1H), 8.31 (s, 1H), 8.25-8.00 (m, 3H), 6.89-6.86 (m, 2H), 6.76 (d, J=8.4 Hz, 1H), 4.29 (d, J=2.4 Hz, 2H), 3.87 (t, J=5.2 Hz, 2H), 2.60 (brs, 2H), 1.69 (s, 3H), 1.61 (s, 3H); MS (ESI) m/z: 349.36 [M+H]⁺; Compounds 56-B (Peak 1) and 56-C (Peak 2) are atropisomers, and the shown stereochemistry may be the opposite of that depicted.

### Example 57

### (S)-3-(4-amino-6-(prop-1-en-2-yl)pyrido[3,4-d]pyrimidin-8-yl)-2,4-dimethylphenol (57-B) and (R)-3-(4-amino-6-(prop-1-en-2-yl)pyrido[3,4-d]pyrimidin-8-yl)-2,4-dimethylphenol (57-C)

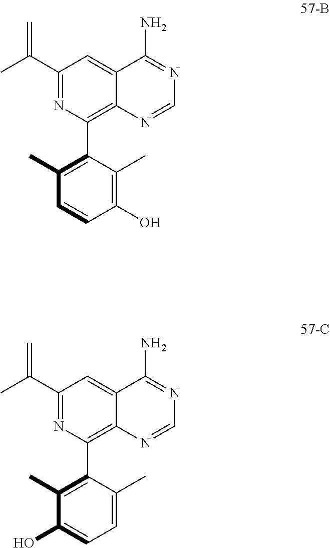

The racemic mixture of 3-(4-amino-6-(prop-1-en-2-yl)pyrido[3,4-d]pyrimidin-8-yl)-2,4-dimethylphenol (300 mg) was prepared following the procedure described in Example 19 and was subjected to chiral SFC purification (Chiralcel OJ-H, 30×250 mm column, 20% MeOH with 0.1% 7N NH₃ in MeOH) then both isomers were repurified by prep-HPLC (C18, ACN/H₂O with 10 mM NH₄HCO₃) to afford the title compounds. From Peak 1: (S)-3-(4-amino-6-(prop-1-en-2-yl)pyrido[3,4-d]pyrimidin-8-yl)-2,4-dimethylphenol (57-B): (78 mg, 24%). Chiral purity 99.8% (Chiralpak OJ-H (4.6×250 mm column), 25% MeOH with 0.5% DEA). ¹H NMR (400 MHz, DMSO-d₆): δ ppm 9.07 (s, 1H), 8.33-8.00 (m, 4H), 6.88 (d, J=8.0 Hz, 1H), 6.76 (d, J=8.4 Hz, 1H), 6.04 (d, J=1.2 Hz, 1H), 5.31 (s, 1H), 2.23 (s, 3H), 1.70 (s, 3H), 1.62 (s, 3H). MS (ESI) m/z: 307.4 [M+H]⁺. From Peak 2: (R)-3-(4-amino-6-(prop-1-en-2-yl)pyrido[3,4-d]pyrimidin-8-yl)-2,4-dimethylphenol (57-C): (75 mg, 23%). Chiral purity 99.5% (Chiralpak OJ-H (4.6×250 mm column), 25% MeOH with 0.5% DEA). ¹H NMR (400 MHz, DMSO-d₆): δ ppm 9.08 (s, 1H), 8.38-7.95 (m, 4H), 6.88 (d, J=8.4 Hz, 1H), 6.76 (d, J=8.0 Hz, 1H), 6.04 (d, J=1.2 Hz, 1H), 5.31 (s, 1H), 2.23 (s, 3H), 1.70 (s, 3H), 1.62 (s, 3H); MS (ESI) m/z: 307.4 [M+H]⁺. Compounds 57-B (Peak 1) and 57-C (Peak 2) are atropisomers, and the shown stereochemistry may be the opposite of that depicted.

### Example 58

### 3-((S)-4-amino-6-((R)-3-fluoropyrrolidin-1-yl)pyrido[3,4-d]pyrimidin-8-yl)-2,4-dimethylphenol (58-B) and 3-((R)-4-amino-6-((R)-3-fluoropyrrolidin-1-yl)pyrido[3,4-d]pyrimidin-8-yl)-2,4-dimethylphenol (58-C)

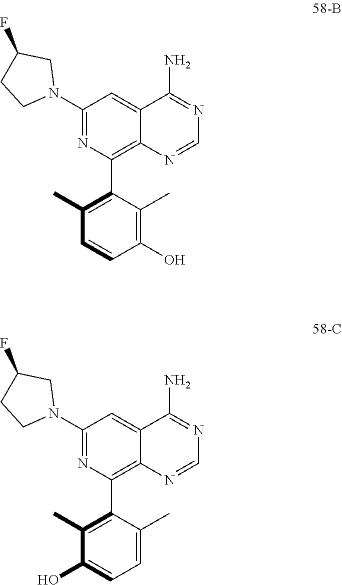

The racemic mixture of 3-(4-amino-6((R)-3-fluoropyrrolidin-1-yl)pyrido[3,4-d]pyrimidin-8-yl)-2,4-dimethylphenol (70 mg) was prepared according to the procedure described in Example 38 and was subjected to chiral SFC purification (ChiralCEL OJ-H, 30×250 mm column, ACN:ⁱPrOH (1:1) with 0.1% ⁱPrNH₂) to afford the title compounds. From Peak 1: 3-((S)-4-amino-6-((R)-3-fluoropyrrolidin-1-yl)pyrido[3,4-d]pyrimidin-8-yl)-2,4-dimethylphenol (58-B): (16.6 mg, 24%). Chiral purity 99.6% (Chiralpak OX—H (4.6×250 mm column), 25% ACN:ⁱPrOH with 0.1% PrNH₂. ¹H NMR (400 MHz, DMSO-d₆): δ ppm 9.03 (s, 1H), 8.03 (s, 1H), 7.74 (br. s, 2H), 6.95 (s, 1H), 6.85 (d, J=8.0 Hz, 1H), 6.73 (d, J=8.0 Hz, 1H), 5.48 (d, J=54.00 Hz, 1H), 3.76-3.56 (m, 3H), 3.49-3.42 (m, 1H), 2.31-2.15 (m, 2H), 1.72 (s, 3H), 1.63 (s, 3H); MS (ESI) m/z: 354.2 [M+H]⁺. From Peak 2: 3-((R)-4-amino-6-((R)-3-fluoropyrrolidin-1-yl)pyrido[3,4-d]pyrimidin-8-yl)-2,4-dimethylphenol (58-C): (22 mg, 31%). Chiral purity 99.7% (Chiralpak OX—H (4.6×250 mm column), 25% ACN:ⁱPrOH with 0.2% DEA. ¹H NMR (400 MHz, DMSO-d₆): δ ppm 9.03 (s, 1H), 8.03 (s, 1H), 7.74 (br. s, 2H), 6.96 (s, 1H), 6.85 (d, J=8.0 Hz, 1H), 6.73 (d, J=8.0 Hz, 1H), 5.48 (d, J=54.00 Hz, 1H), 3.76-3.57 (m, 3H), 3.49-3.42 (m, 1H), 2.26-2.15 (m, 2H), 1.73 (s, 3H), 1.63 (s, 3H); MS (ESI) m/z: 354.2 [M+H]⁺; Compounds 58-B (Peak 1) and 58—C (Peak 2) are atropisomers, and the shown stereochemistry may be the opposite of that depicted.

### Example 59

### 3-((S)-4-Amino-6-((S)-3-fluoropyrrolidin-1-yl)pyrido[3,4-d]pyrimidin-8-yl)-2,4-dimethylphenol (59-B) and 3-((R)-4-Amino-6-((S)-3-fluoropyrrolidin-1-yl)pyrido[3,4-d]pyrimidin-8-yl)-2,4-dimethylphenol (59-C)

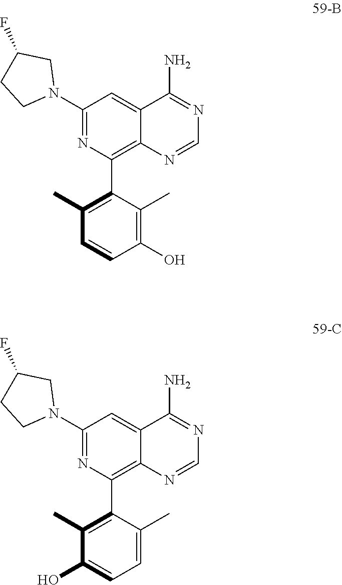

The racemic mixture of 3-(4-amino-6-((S)-3-fluoropyrrolidin-1-yl)pyrido[3,4-d]pyrimidin-8-yl)-2,4-dimethylphenol (38 mg) was prepared according to the procedure described in Example 38 and was subjected to chiral SFC purification (Chiralcel OJ-H, 30×250 mm column, ACN:MeOH (1:1) with 0.1% 7N NH₃ in MeOH) to afford the title compounds. From Peak 1: 3-((S)-4-amino-6-((S)-3-fluoropyrrolidin-1-yl)pyrido[3,4-d]pyrimidin-8-yl)-2,4-dimethylphenol (59-B): (8.2 mg, 3%). Chiral purity 99.9% (Chiralpak OJ-H (4.6×250 mm column), 25% ACN:MeOH with 0.2% 7N NH₃ in MeOH. ¹H NMR (400 MHz, DMSO-d₆): δ ppm 9.05 (s, 1H), 8.03 (s, 1H), 7.74 (br. s, 2H), 6.96 (s, 1H), 6.85 (d, J=8.0 Hz, 1H), 6.74 (d, J=8.4 Hz, 1H), 5.48 (d, J=54.0 Hz, 1H), 3.76-3.55 (m, 3H), 3.53-3.40 (m, 1H), 2.34-2.13 (m, 2H), 1.73 (s, 3H), 1.64 (s, 3H). MS (ESI) m/z: 354.7 [M+H]⁺. From Peak 2: 3-((R)-4-amino-6-((S)-3-fluoropyrrolidin-1-yl)pyrido[3,4-d]pyrimidin-8-yl)-2,4-dimethylphenol (59-C): (8.7 mg, 3.5%). Chiral purity 99.0% (Chiralpak OX—H (4.6×250 mm column), 25% ACN:MeOH with 0.2% 7N NH₃ in MeOH. ¹H NMR (400 MHz, DMSO-d₆): δ ppm 9.04 (s, 1H), 8.03 (s, 1H), 7.74 (br. s, 2H), 6.96 (s, 1H), 6.85 (d, J=8.0 Hz, 1H), 6.74 (d, J=8.4 Hz, 1H), 5.48 (d, J=54.0 Hz, 1H), 3.76-3.55 (m, 3H), 3.53-3.40 (m, 1H), 2.34-2.13 (m, 2H), 1.73 (s, 3H), 1.64 (s, 3H); MS (ESI) m/z: 354.3 [M+H]⁺; Compounds 59-B (Peak 1) and 59-C (Peak 2) are atropisomers, and the shown stereochemistry may be the opposite of that depicted.

### Example 60

### (S)-3-(4-amino-6-(cyclopropyl(methyl-d3)amino)pyrido[3,4-d]pyrimidin-8-yl)-2,4-dimethylphenol (60-B) and (R)-3-(4-amino-6-(cyclopropyl(methyl-d3)amino)pyrido[3,4-d]pyrimidin-8-yl)-2,4-dimethylphenol (60-C)

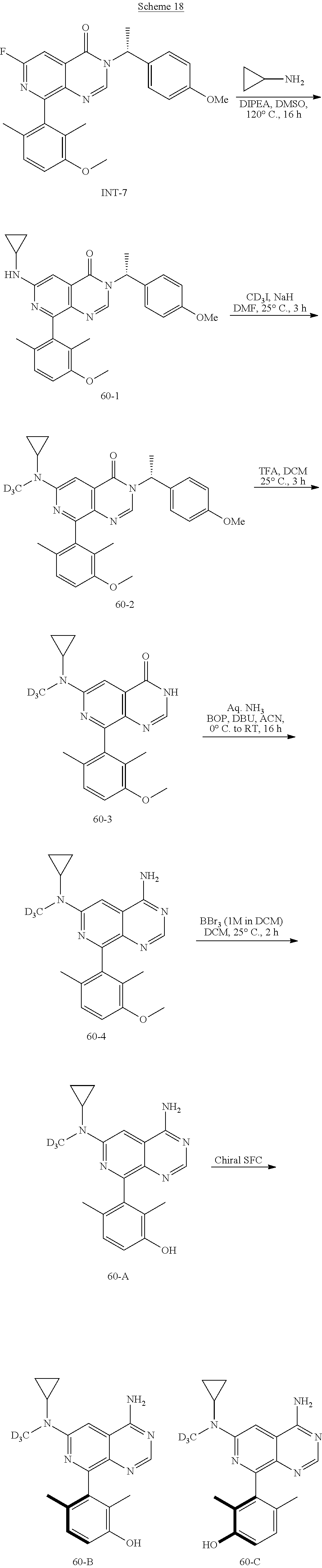

### 6-(cyclopropylamino)-8-(3-methoxy-2,6-dimethylphenyl)-3-((R)-1-(4-methoxyphenyl)ethyl)pyrido[3,4-d]pyrimidin-4 (3H)-one (60-1)

To a stirred solution of 6-fluoro-8-(3-methoxy-2,6-dimethylphenyl)-3-((R)-1-(4-methoxyphenyl)ethyl)pyrido[3,4-d]pyrimidin-4 (3H)-one (INT-7) (0.82, g, 1.89 mmol) in DMSO (8.2 mL) were added cyclopropylamine (1.08 mL, 18.9 mmol) and N,N-Diisopropylethylamine (3.29 mL, 18.9 mmol) at 25° C. After stirring at 120° C. for 16 h, the reaction was quenched with water (100 mL) and extracted with EA (2×200 mL). The combined organic layers washed with brine (100 mL), dried over magnesium sulfate, filtered and concentrated. The residue purified by flash column chromatography (SiO₂, EA/PET gradient) to afford 6-(cyclopropylamino)-8-(3-methoxy-2,6-dimethylphenyl)-3-((R)-1-(4-methoxyphenyl)ethyl)pyrido[3,4-d]pyrimidin-4 (3H)-one (60-1) (0.53 g, 60%). MS (ESI) 471.40 [M+H]⁺.

### 6-(cyclopropyl(methyl-d3)amino)-8-(3-methoxy-2,6-dimethylphenyl)-3-((R)-1-(4-methoxyphenyl)ethyl)pyrido[3,4-d]pyrimidin-4 (3H)-one (60-2)

To a stirred solution of 6-(cyclopropylamino)-8-(3-methoxy-2,6-dimethylphenyl)-3-((R)-1-(4-methoxyphenyl)ethyl)pyrido[3,4-d]pyrimidin-4 (3H)-one (60-1) (0.50 g, 1.06 mmol) in dimethylformamide (5.0 mL) was added sodium hydride (60% dispersion in mineral oil, 0.1 g, 3.19 mmol) at 0° C., stirred at 25° C. for 30 min, followed by addition of iodomethane-d3 (0.20 mL, 3.19 mmol). After stirring at 25° C. for 3 h, the reaction was quenched with ice cold water (100 mL) and extracted with EA (2×100 mL). The combined organic layers washed with brine (50 mL), dried over magnesium sulfate, filtered and concentrated. The residue purified by flash column chromatography (SiO₂, EA/PET gradient) to afford 6-(cyclopropyl(methyl-d3)amino)-8-(3-methoxy-2,6-dimethylphenyl)-3-((R)-1-(4-methoxyphenyl)ethyl)pyrido[3,4-d]pyrimidin-4 (3H)-one (60-2) (0.34 g, 66%). MS (ESI) 488.35 [M+H]⁺.

### 6-(cyclopropyl(methyl-d3)amino)-8-(3-methoxy-2,6-dimethylphenyl)pyrido[3,4-d]pyrimidin-4 (3H)-one (60-3)

To a stirred solution of 6-(cyclopropyl(methyl-d3)amino)-8-(3-methoxy-2,6-dimethylphenyl)-3-((R)-1-(4-methoxyphenyl)ethyl)pyrido[3,4-d]pyrimidin-4 (3H)-one (60-2) (0.509 g, 1.02 mmol) in dichloromethane (5.0 mL) was added trifluoroacetic acid (2.5 mL) at 25° C. After stirring at 25° C. for 3 h, the reaction was quenched with aqueous sodium bicarbonate solution (100 mL) and extracted with EA (3×100 mL). The combined organic layers washed with brine (100 mL), dried over sodium sulfate, filtered and concentrated. The residue purified by flash column chromatography (SiO₂, EA/PET gradient) to afford 6-(cyclopropyl(methyl-d3)amino)-8-(3-methoxy-2,6-dimethylphenyl)pyrido[3,4-d]pyrimidin-4 (3H)-one (60-3) (0.34 g, 94%). MS (ESI) 354.35 [M+H]⁺.

### N⁶-cyclopropyl-8-(3-methoxy-2,6-dimethylphenyl)-N⁶-(methyl-d3)pyrido[3,4-d]pyrimidine-4,6-diamine (60-4)

To a stirred solution of 6-(cyclopropyl(methyl-d3)amino)-8-(3-methoxy-2,6-dimethylphenyl)pyrido[3,4-d]pyrimidin-4 (3H)-one (60-3) (0.329 g, 0.905 mmol) and BOP reagent (0.80-4 g, 1.81 mmol) in acetonitrile (6.4 mL) was added DBU (0.3, 2.72 mmol) dropwise at 0° C., stirred 0° C. for 2 h, followed by addition of aqueous ammonia (3.2 mL). After stirring at 25° C. for 16 h, the reaction was quenched with water (100 mL) and extracted with EA (2×100 mL). The combined organic layers washed with brine (50 mL), dried over magnesium sulfate, filtered and concentrated. The residue purified by flash column chromatography (SiO₂, EA/PET gradient) to afford N⁶-cyclopropyl-8-(3-methoxy-2,6-dimethylphenyl)-N⁶-(methyl-d3)pyrido[3,4-d]pyrimidine-4,6-diamine (60-4) (0.25 g, 78%). MS (ESI) 353.28 [M+H]⁺.

### 3-(4-amino-6-(cyclopropyl(methyl-d3)amino)pyrido[3,4-d]pyrimidin-8-yl)-2,4-dimethylphenol (60-A)

To a stirred solution of N⁶-cyclopropyl-8-(3-methoxy-2,6-dimethylphenyl)-N⁶-(methyl-d3)pyrido[3,4-d]pyrimidine-4,6-diamine (60-4) (0.200 g, 0.567 mmol) in dichloromethane (10.0 mL) was added BBr₃ (1M in DCM) (2.27 mL, 2.27 mmol) at 0° C. After stirring at 25° C. for 2 h, the reaction was quenched with water (100 mL) and extracted with EA (2×100 mL). The combined organic layers washed with brine (50 mL), dried over magnesium sulfate, filtered and concentrated. The residue purified by flash column chromatography (SiO₂, methanol/dichloromethane gradient) to afford 3-(4-amino-6-(cyclopropyl(methyl-d3)amino)pyrido[3,4-d]pyrimidin-8-yl)-2,4-dimethylphenol (60-A) (0.12 g, 62%). MS (ESI) 339.25 [M+H]⁺.

### (S)-3-(4-amino-6-(cyclopropyl(methyl-d3)amino)pyrido[3,4-d]pyrimidin-8-yl)-2,4-dimethylphenol (60-B) and (R)-3-(4-amino-6-(cyclopropyl(methyl-d3)amino)pyrido[3,4-d]pyrimidin-8-yl)-2,4-dimethylphenol (60-C)

3-(4-amino-6-(cyclopropyl(methyl-d3)amino)pyrido[3,4-d]pyrimidin-8-yl)-2,4-dimethylphenol (60-A) (120 mg) was separated by chiral SFC (Chiralpak AD-H (30×250) mm, 5p, CO₂/0.2% isopropylamine in IPA) to afford (S)-3-(4-amino-6-(cyclopropyl(methyl-d3)amino)pyrido[3,4-d]pyrimidin-8-yl)-2,4-dimethylphenol (60-B) (30 mg, 25% yield, 97.82% ee) and (R)-3-(4-amino-6-(cyclopropyl(methyl-d3)amino)pyrido[3,4-d]pyrimidin-8-yl)-2,4-dimethylphenol (60-C) (35 mg, 29% yield, 90.35% ee). Compounds 60-B and 60-C are atropisomers, and the shown stereochemistry may be the opposite of that depicted. 60-B: ¹H NMR (400 MHz, DMSO-d₆): δ ppm 9.02 (s, 1H), 8.06 (s, 1H), 7.81 (brs, 2H), 7.31 (s, 1H), 6.85 (d, J=8.00 Hz, 1H), 6.73 (d, J=8.00 Hz, 1H), 2.52-2.51 (m, 1H), 1.72 (s, 3H), 1.63 (s, 3H), 0.97-0.95 (m, 2H), 0.60-0.61 (m, 2H); MS (ESI) 339.33 [M+H]⁺; HPLC: 99.05%, 60-C: ¹H NMR (400 MHz, DMSO-d₆): δ ppm 9.02 (s, 1H), 8.06 (s, 1H), 7.81 (s, 2H), 7.31 (brs, 1H), 6.85 (d, J=8.00 Hz, 1H), 6.73 (d, J=8.00 Hz, 1H), 2.50-2.49 (m, 1H), 1.72 (s, 3H), 1.63 (s, 3H), 0.97-0.96 (m, 2H), 0.62-0.61 (m, 2H); MS (ESI) 339.34 [M+H]⁺; HPLC: 99.48%.

### Example 61

### 3-((S)-6-(((1R,5S,6r)-3-oxabicyclo[3.1.0]hexan-6-yl)(methyl)amino)-4-aminopyrido[3,4-d]pyrimidin-8-yl)-2,4-dimethylphenol (61-B) and 3-((R)-6-(((1R,5S,6r)-3-oxabicyclo[3.1.0]hexan-6-yl)(methyl)amino)-4-aminopyrido[3,4-d]pyrimidin-8-yl)-2,4-dimethylphenol (61-C)

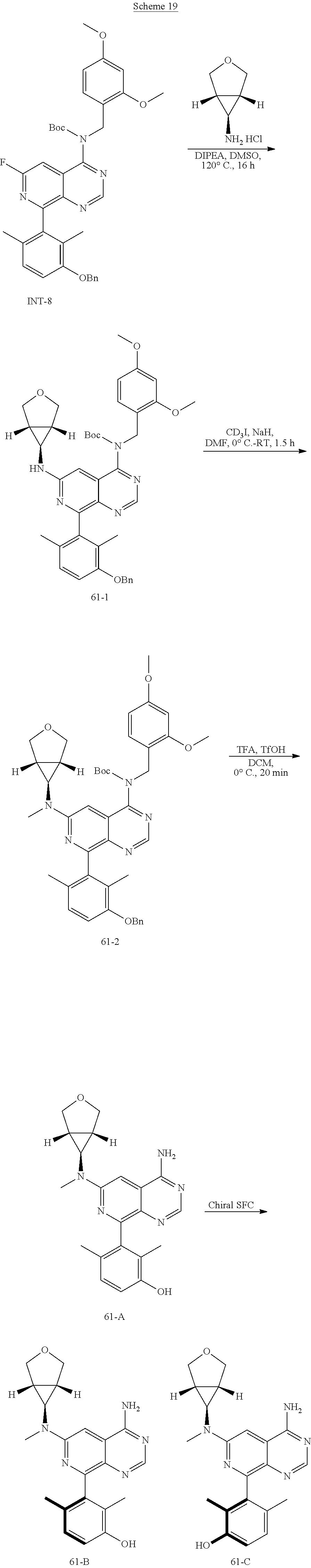

### tert-butyl (6-(((1R,5S,6r)-3-oxabicyclo[3.1.0]hexan-6-yl)amino)-8-(3-(benzyloxy)-2,6-dimethylphenyl)pyrido[3,4-d]pyrimidin-4-yl)(2,4-dimethoxybenzyl)carbamate (61-1)

To a stirred solution of tert-butyl (8-(3-(benzyloxy)-2,6-dimethylphenyl)-6-fluoropyrido[3,4-d]pyrimidin-4-yl)(2,4-dimethoxybenzyl)carbamate (INT-8) (0.300 g, 0.48 mmol) in DMSO (3.0 mL) were added N,N-Diisopropylethylamine (1.67 mL, 9.60 mmol) and (1R,5S,6r)-3-oxabicyclo[3.1.0]hexan-6-amine hydrochloride (0.324 g, 2.40 mmol) at rt. After stirring at 120° C. for 16 h, the mixture was diluted with ice cold water (30 mL) and extracted with EtOAc (2×30 mL). The combined organic layer washed with brine, dried over MgSO₄, filtered and concentrated. The residue purified by flash column chromatography (SiO₂, EA/PET) to afford tert-butyl (6-(((1R,5S,6r)-3-oxabicyclo[3.1.0]hexan-6-yl)amino)-8-(3-(benzyloxy)-2,6-dimethylphenyl)pyrido[3,4-d]pyrimidin-4-yl)(2,4-dimethoxybenzyl)carbamate (61-1) (0.20 g, 59%). MS (ESI) 704.63 [M+H]⁺.

### tert-butyl (6-(((1R,5S,6r)-3-oxabicyclo[3.1.0]hexan-6-yl)(methyl)amino)-8-(3-(benzyloxy)-2,6-dimethylphenyl)pyrido[3,4-d]pyrimidin-4-yl)(2,4-dimethoxybenzyl)carbamate (61-2)

To a stirred solution of tert-butyl (6-(((1R,5S,6r)-3-oxabicyclo[3.1.0]hexan-6-yl)amino)-8-(3-(benzyloxy)-2,6-dimethylphenyl)pyrido[3,4-d]pyrimidin-4-yl)(2,4-dimethoxybenzyl)carbamate (61-1) (0.20 g, 0.284 mmol) in DMF (2.0 mL) was added sodium hydride (60% dispersion in Paraffin Liquid, 1.14 mmol) at 0° C. The mixture stirred at 0° C. for 30 min and then iodomethane (53 μL, 0.852 mmol) was added. After stirring at rt for 1.5 h, the reaction was quenched with ice cold water (20 mL) and extracted with EtOAc (2×20 mL). The combined organic layers washed with brine, dried over MgSO₄, filtered and concentrated. The residue purified by flash column chromatography (SiO₂, EA/PET gradient) to afford tert-butyl (6-(((1R,5S,6r)-3-oxabicyclo[3.1.0]hexan-6-yl)(methyl)amino)-8-(3-(benzyloxy)-2,6-dimethylphenyl)pyrido[3,4-d]pyrimidin-4-yl)(2,4-dimethoxybenzyl)carbamate (61-2) (0.115 g, 56%). MS (ESI) 718.68 [M+H]⁺.

### 3-(6-(((1R,5S,6r)-3-oxabicyclo[3.1.0]hexan-6-yl)(methyl)amino)-4-aminopyrido[3,4-d]pyrimidin-8-yl)-2,4-dimethylphenol (61-A)

To a stirred solution of tert-butyl (6-(((1R,5S,6r)-3-oxabicyclo[3.1.0]hexan-6-yl)(methyl)amino)-8-(3-(benzyloxy)-2,6-dimethylphenyl)pyrido[3,4-d]pyrimidin-4-yl)(2,4-dimethoxybenzyl)carbamate (0.14 g, 0.195 mmol) in dichloromethane (4.2 mL) were added trifluoroacetic acid (0.28 mL) and triflic acid (0.14 mL) at 0° C. After stirring at 0° C. for 20 min, the mixture was diluted with EtOAc (30 mL), washed with saturated sodium bicarbonate solution (20 mL), water (2×10 mL), brine, dried over magnesium sulfate, filtered, and concentrated. The residue purified by prep-HPLC to afford 3-(6-(((1R,5S,6r)-3-oxabicyclo[3.1.0]hexan-6-yl)(methyl)amino)-4-aminopyrido[3,4-d]pyrimidin-8-yl)-2,4-dimethylphenol (61-A) (40 mg, 54%).

### 3-((S)-6-(((1R,5S,6r)-3-oxabicyclo[3.1.0]hexan-6-yl)(methyl)amino)-4-aminopyrido[3,4-d]pyrimidin-8-yl)-2,4-dimethylphenol (61-B) and 3-((R)-6-(((1R,5S,6r)-3-oxabicyclo[3.1.0]hexan-6-yl)(methyl)amino)-4-aminopyrido[3,4-d]pyrimidin-8-yl)-2,4-dimethylphenol (61-C)

3-(6-(((1R,5S,6r)-3-oxabicyclo[3.1.0]hexan-6-yl)(methyl)amino)-4-aminopyrido[3,4-d]pyrimidin-8-yl)-2,4-dimethylphenol (61-A) (40 mg, 0.106 mmol) was subjected to chiral SFC (LUX CELLULOSE-4 (30×250) mm, 5μ, CO₂/0.1% isopropylamine in ACN:IPA (8:2)), concentrated and lyophilized to afford 3-((S)-6-(((1R,5S,6r)-3-oxabicyclo[3.1.0]hexan-6-yl)(methyl)amino)-4-aminopyrido[3,4-d]pyrimidin-8-yl)-2,4-dimethylphenol (61-B) (12.8 mg, 31.9%, 99.27% ee) and 3-((R)-6-(((1R,5S,6r)-3-oxabicyclo[3.1.0]hexan-6-yl)(methyl)amino)-4-aminopyrido[3,4-d]pyrimidin-8-yl)-2,4-dimethylphenol (61-C) (12.9 mg, 32.3%, 97.39% ee). Compounds 61-B and 61-C are atropisomers, and the shown stereochemistry may be the opposite of that depicted. 61-B: ¹H NMR (400 MHz, DMSO-d₆): δ ppm 9.03 (s, 1H), 8.07 (s, 1H), 7.78 (brs, 2H), 7.11 (s, 1H), 6.85 (d, J=8.0 Hz, 1H), 6.73 (d, J=8.0 Hz, 1H), 4.17-4.14 (m, 2H), 3.68-3.66 (m, 2H), 3.07 (s, 3H), 2.40 (s, 1H), 1.98 (s, 2H), 1.72 (s, 3H), 1.63 (s, 3H); MS (ESI) 378.33 [M+H]⁺; HPLC: 99.68%. 61-C: ¹H NMR (400 MHz, DMSO-d₆): δ ppm 9.03 (s, 1H), 8.07 (s, 1H), 7.78 (brs, 2H), 7.11 (s, 1H), 6.85 (d, J=8.0 Hz, 1H), 6.73 (d, J=8.0 Hz, 1H), 4.17-4.13 (m, 2H), 3.68-3.66 (m, 2H), 3.07 (s, 3H), 2.40 (s, 1H), 1.98 (s, 2H), 1.72 (s, 3H), 1.63 (s, 3H); MS (ESI) 378.32 [M+H]⁺; HPLC: 99.70%.

### Example 62

### 3-((S)-4-amino-6-(((1s,3R)-3-methoxycyclobutyl)(methyl)amino)pyrido[3,4-d]pyrimidin-8-yl)-2,4-dimethylphenol (62-B), 3-((R)-4-amino-6-(((1s,3S)-3-methoxycyclobutyl)(methyl)amino)pyrido[3,4-d]pyrimidin-8-yl)-2,4-dimethylphenol (62-C), 3-((S)-4-amino-6-(((1r,3S)-3-methoxycyclobutyl)(methyl)amino)pyrido[3,4-d]pyrimidin-8-yl)-2,4-dimethylphenol (62-D) and 3-((R)-4-amino-6-(((1r,3R)-3-methoxycyclobutyl)(methyl)amino)pyrido[3,4-d]pyrimidin-8-yl)-2,4-dimethylphenol (62-E)

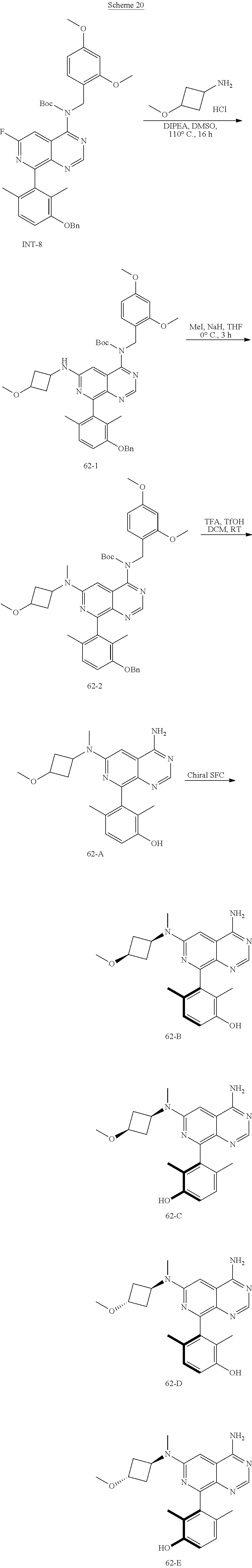

### tert-butyl (8-(3-(benzyloxy)-2,6-dimethylphenyl)-6-((3-methoxycyclobutyl)amino)pyrido[3,4-d]pyrimidin-4-yl)(2,4-dimethoxybenzyl)carbamate (62-1)

To a stirred solution of tert-butyl (8-(3-(benzyloxy)-2,6-dimethylphenyl)-6-fluoropyrido[3,4-d]pyrimidin-4-yl)(2,4-dimethoxybenzyl)carbamate (61-4) (0.200 g, 0.320 mmol) in DMSO (2 mL) were added N,N-Diisopropylethylamine (0.558 mL, 3.20 mmol) and 3-methoxycyclobutan-1-amine hydrochloride (176 mg, 1.28 mmol). After stirring at 80° C. for 16 h, it was diluted with water (50 mL), extracted with EA (2×50 mL), combined organic layers washed with brine (30×2 mL), dried over sodium sulphate, filtered, concentrated, and the residue purified by flash column chromatography (SiO₂, EA/PET) to yield tert-butyl (8-(3-(benzyloxy)-2,6-dimethylphenyl)-6-((3-methoxycyclobutyl)amino)pyrido[3,4-d]pyrimidin-4-yl)(2,4-dimethoxybenzyl)carbamate (62-1) (80 mg, 35%). MS (ESI) 706.46 [M+H]⁺.

### tert-butyl (8-(3-(benzyloxy)-2,6-dimethylphenyl)-6-((3-methoxycyclobutyl)(methyl)amino)pyrido[3,4-d]pyrimidin-4-yl)(2,4-dimethoxybenzyl)carbamate (62-2)

To a stirred solution of tert-butyl (8-(3-(benzyloxy)-2,6-dimethylphenyl)-6-((3-methoxycyclobutyl)amino)pyrido[3,4-d]pyrimidin-4-yl)(2,4-dimethoxybenzyl)carbamate (62-1) (0.350 g, 0.496 mmol) in DM (3.5 mL), was added sodium hydride (0.079 g, 1.98 mmol) at 0° C., stirred at 0° C. for 10 min, then added iodomethane (0.093 mL, 1.49 mmol). After stirring at RT for 2 h, it was quenched with ice cold water (20 mL), extracted with EA (2×20 mL), combined organic layers washed with brine (20 mL), dried over magnesium sulphate, filtered and concentrated to afford tert-butyl (8-(3-(benzyloxy)-2,6-dimethylphenyl)-6-((3-methoxycyclobutyl)(methyl)amino)pyrido[3,4-d]pyrimidin-4-yl)(2,4-dimethoxybenzyl)carbamate (62-2) (crude 400 mg), which was used directly for next step without further purification. MS (ESI) 720.49 [M+H]⁺.

### tert-butyl (8-(3-(benzyloxy)-2,6-dimethylphenyl)-6-((3-methoxycyclobutyl)(methyl)amino)pyrido[3,4-d]pyrimidin-4-yl)(2,4-dimethoxybenzyl)carbamate (62-A)

To a stirred solution of tert-butyl (8-(3-(benzyloxy)-2,6-dimethylphenyl)-6-((3-methoxycyclobutyl)(methyl)amino)pyrido[3,4-d]pyrimidin-4-yl)(2,4-dimethoxybenzyl)carbamate (62-2) (0.400 g, 0.556 mmol) (crude) in DCM (12 mL) were added trifluoroacetic acid (0.80 mL) and triflic acid (0.40 mL) at 0° C. After stirring at 0° C. for 20 min, it was cooled, carefully neutralized with saturated sodium bicarbonate solution (5 mL), extracted with EA (2×10 ml), combined organic layers washed with brine, dried over MgSO₄, filtered, concentrated, and the residue purified by flash column chromatography (SiO₂, methanol/dichloromethane) to 3-(4-amino-6-((3-methoxycyclobutyl)(methyl)amino)pyrido[3,4-d]pyrimidin-8-yl)-2,4-dimethylphenol (62-A) (115 mg, 54% yield). MS (ESI) 380.29 [M+H]⁺.

### 3-((S)-4-amino-6-(((1s,3R)-3-methoxycyclobutyl)(methyl)amino)pyrido[3,4-d]pyrimidin-8-yl)-2,4-dimethylphenol (62-B), 3-((R)-4-amino-6-(((1s,3S)-3-methoxycyclobutyl)(methyl)amino)pyrido[3,4-d]pyrimidin-8-yl)-2,4-dimethylphenol (62-C), 3-((S)-4-amino-6-(((1r,3S)-3-methoxycyclobutyl)(methyl)amino)pyrido[3,4-d]pyrimidin-8-yl)-2,4-dimethylphenol (62-D) and 3-((R)-4-amino-6-(((1r,3R)-3-methoxycyclobutyl)(methyl)amino)pyrido[3,4-d]pyrimidin-8-yl)-2,4-dimethylphenol (62-E)

3-(4-amino-6-((3-methoxycyclobutyl)(methyl)amino)pyrido[3,4-d]pyrimidin-8-yl)-2,4-dimethylphenol (62-A) (115 mg) were separated by the first chiral SFC (Chiralpak IC (30×250) mm, 5p, CO₂ (0.1% DEA in MeOH) to give two sets of isomers. Each set of isomers were separated by the second chiral SFC (Chiralcel OX—H (30×250) mm, 5p, CO₂ (0.2% DEA in MeOH), to afford 3-((S)-4-amino-6-(((1s,3R)-3-methoxycyclobutyl)(methyl)amino)pyrido[3,4-d]pyrimidin-8-yl)-2,4-dimethylphenol (62-B) (17 mg, 15%, 99.92% ee), 3-((R)-4-amino-6-(((1s,3S)-3-methoxycyclobutyl)(methyl)amino)pyrido[3,4-d]pyrimidin-8-yl)-2,4-dimethylphenol (62-C) (15 mg, 13%, 95.91% ee), 3-((S)-4-amino-6-(((1r,3S)-3-methoxycyclobutyl)(methyl)amino)pyrido[3,4-d]pyrimidin-8-yl)-2,4-dimethylphenol (62-D) (4.7 mg, 4%, 99.34% ee), and 3-((R)-4-amino-6-(((1r,3R)-3-methoxycyclobutyl)(methyl)amino)pyrido[3,4-d]pyrimidin-8-yl)-2,4-dimethylphenol (62-E) (7.7 mg, 7%, 93.14% ee). Compounds 62-B, 62-C, 62-D and 62-E are atropisomers, and the stereochemistry may be the opposite of that depicted. 62-B: ¹H NMR (400 MHz, DMSO-d₆): δ 9.03 (brs, 2H), 8.03 (s, 1H), 7.76 (brs, 2H), 7.03 (s, 1H), 6.85 (d, J=8.4 Hz, 1H), 6.73 (d, J=8.0 Hz, 1H), 4.53-4.45 (m, 1H), 3.68-3.58 (m, 1H), 3.15 (s, 3H), 2.99 (s, 3H), 2.58-2.54 (m, 2H), 2.02-1.95 (m, 2H), 1.71 (s, 3H), 1.62 (s, 3H); MS (ESI) 380.34 [M+H]⁺; Purity by HPLC: 99.69%. 62-C: ¹H NMR (400 MHz, DMSO-d₆): δ 9.03 (br s, 1H), 8.03 (s, 1H), 7.76 (brs, 2H), 7.03 (s, 1H), 6.85 (d, J=8.0 Hz, 1H), 6.73 (d, J=8.0 Hz, 1H), 4.53-4.45 (m, 1H), 3.65-3.58 (m, 1H), 3.15 (s, 3H), 2.99 (s, 3H), 2.59-2.49 (m, 2H), 2.02-1.95 (m, 2H), 1.71 (s, 3H), 1.62 (s, 3H); MS (ESI) 380.34 [M+H]⁺; Purity by HPLC: 99.64%. 62-D: ¹H NMR (400 MHz, DMSO-d₆): δ 9.03 (s, 1H), 8.03 (s, 1H), 7.77 (brs, 2H), 6.99 (s, 1H), 6.85 (d, J=8.4 Hz, 1H), 6.73 (d, J=8.0 Hz, 1H), 5.16-5.08 (m, 1H), 3.92-3.88 (m, 1H), 3.16 (s, 3H), 3.01 (s, 3H), 2.39-2.32 (m, 2H), 2.27-2.21 (m, 2H), 1.71 (s, 3H), 1.62 (s, 3H); MS (ESI) 380.34 [M+H]⁺; Purity by HPLC: 99.60%. 62-E: ¹H NMR (400 MHz, DMSO-d₆): δ 9.03 (s, 1H), 8.03 (s, 1H), 7.77 (brs, 2H), 6.99 (s, 1H), 6.85 (d, J=8.4 Hz, 1H), 6.73 (d, J=8.0 Hz, 1H), 5.14-5.08 (m, 1H), 3.92-3.89 (m, 1H), 3.16 (s, 3H), 3.01 (s, 3H), 2.36-2.32 (m, 2H), 2.27-2.21 (m, 2H), 1.71 (s, 3H), 1.62 (s, 3H); MS (ESI) 380.34 [M+H]⁺; Purity by HPLC: 99.81%.

### Example 63

### 3-((S)-6-(((1R,5S,6r)-3-oxabicyclo[3.1.0]hexan-6-yl)(methyl-d₃)amino)-4-aminopyrido[3,4-d]pyrimidin-8-yl)-2,4-dimethylphenol (63-B) and 3-((R)-6-(((1R,5S,6r)-3-oxabicyclo[3.1.0]hexan-6-yl)(methyl-d₃)amino)-4-aminopyrido[3,4-d]pyrimidin-8-yl)-2,4-dimethylphenol (63-C)

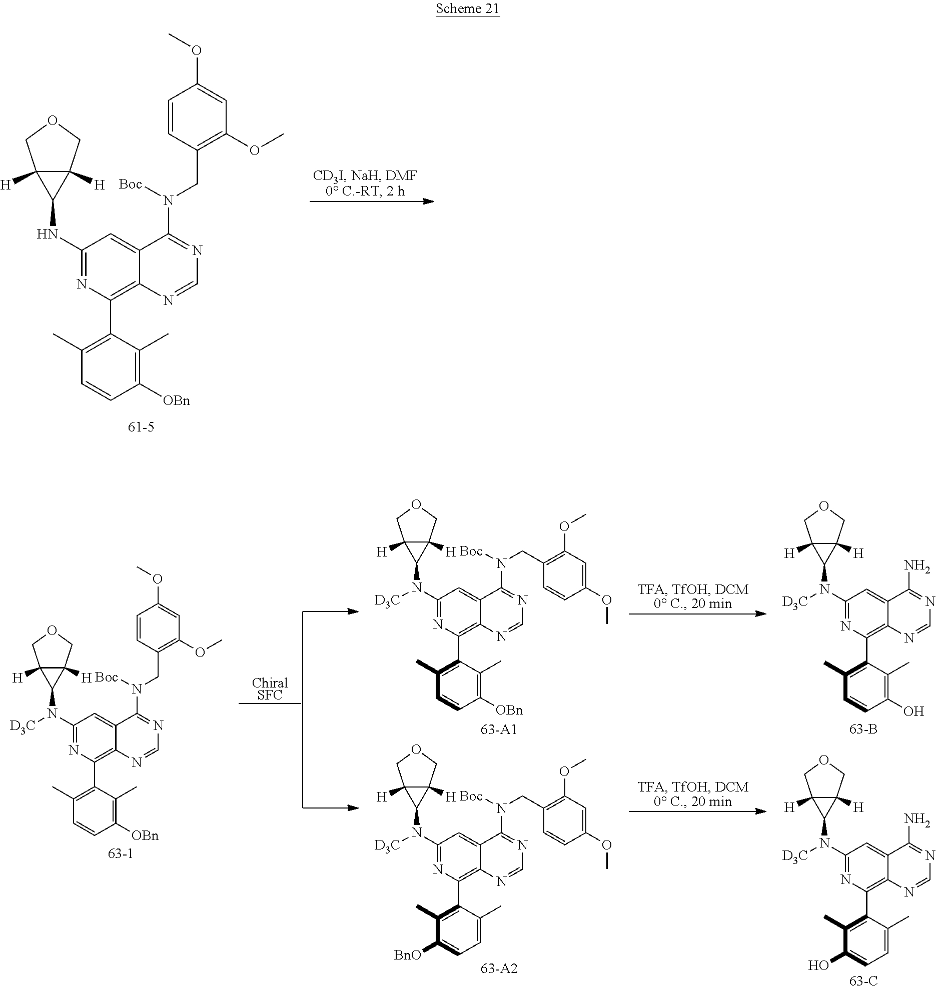

### tert-butyl (6-(((1R,5S,6r)-3-oxabicyclo[3.1.0]hexan-6-yl)(methyl-d₃)amino)-8-(3-(benzyloxy)-2,6-dimethylphenyl)pyrido[3,4-d]pyrimidin-4-yl)(2,4-dimethoxybenzyl)carbamate (63-1)

To a stirred solution of tert-butyl (6-(((1R,5S,6r)-3-oxabicyclo[3.1.0]hexan-6-yl)amino)-8-(3-(benzyloxy)-2,6-dimethylphenyl)pyrido[3,4-d]pyrimidin-4-yl)(2,4-dimethoxybenzyl)carbamate (61-5) (1.00 g, 1.42 mmol) in dimethylformamide (9 mL), was added sodium hydride (60%, dispersion in paraffin liquid, 0.170 g, 4.262 mmol) at 0° C., stirred at 0° C. for 30 min, then iodomethane-d₃ (0.265 mL, 4.26 mmol) added to at 0° C. After stirring at RT for 1.5 h, it was quenched with ice cold water (50 mL), extracted with EA (2×5 mL), combined organic layers washed with brine, dried over MgSO₄, filtered, concentrated, and the residue purified by flash column chromatography (SiO₂, EA/PET) to afford tert-butyl (6-(((1R,5S,6r)-3-oxabicyclo[3.1.0]hexan-6-yl)(methyl-d₃)amino)-8-(3-(benzyloxy)-2,6-dimethylphenyl)pyrido[3,4-d]pyrimidin-4-yl)(2,4-dimethoxybenzyl)carbamate (63-1) (0.91 g, 88%). MS (ESI) 721.74 [M+H]⁺. tert-butyl ((S)-6-(((1R,5S,6r)-3-oxabicyclo[3.1.0]hexan-6-yl)(methyl-d₃)amino)-8-(3-(benzyloxy)-2,6-dimethylphenyl)pyrido[3,4-d]pyrimidin-4-yl)(2,4-dimethoxybenzyl)carbamate (63-A1) and tert-butyl ((R)-6-(((1R,5S,6r)-3-oxabicyclo[3.1.0]hexan-6-yl)(methyl-d₃)amino)-8-(3-(benzyloxy)-2,6-dimethylphenyl)pyrido[3,4-d]pyrimidin-4-yl)(2,4-dimethoxybenzyl)carbamate (63-A2)

tert-butyl (6-(((1R,5S,6r)-3-oxabicyclo[3.1.0]hexan-6-yl)(methyl-d₃)amino)-8-(3-(benzyloxy)-2,6-dimethylphenyl)pyrido[3,4-d]pyrimidin-4-yl)(2,4-dimethoxybenzyl)carbamate (63-1) (0.90 g) was subjected to chiral SFC (CHIRALPAK AD-H (30×250) mm, 5p, CO₂/0.1% isopropylamine in IPA) to afford tert-butyl ((S)-6-(((1R,5S,6r)-3-oxabicyclo[3.1.0]hexan-6-yl)(methyl-d₃)amino)-8-(3-(benzyloxy)-2,6-dimethylphenyl)pyrido[3,4-d]pyrimidin-4-yl)(2,4-dimethoxybenzyl)carbamate (63-A1) (390 mg, 43%), and tert-butyl ((R)-6-(((1R,5S,6r)-3-oxabicyclo[3.1.0]hexan-6-yl)(methyl-d₃)amino)-8-(3-(benzyloxy)-2,6-dimethylphenyl)pyrido[3,4-d]pyrimidin-4-yl)(2,4-dimethoxybenzyl)carbamate (63-A2) (380 mg, 42%). 63-A1: MS (ESI) 721.55 [M+H]⁺; chiral purity: 99.1% ee. 63-A2: MS (ESI) 721.55 [M+H]⁺; chiral purity: 97.4% ee.

### 3-((S)-6-(((1R,5S,6r)-3-oxabicyclo[3.1.0]hexan-6-yl)(methyl-d₃)amino)-4-aminopyrido[3,4-d]pyrimidin-8-yl)-2,4-dimethylphenol (63-B)

To a stirred solution of tert-butyl ((S)-6-(((1R,5S,6r)-3-oxabicyclo[3.1.0]hexan-6-yl)(methyl-d₃)amino)-8-(3-(benzyloxy)-2,6-dimethylphenyl)pyrido[3,4-d]pyrimidin-4-yl)(2,4-dimethoxybenzyl)carbamate (63-A1) (0.350 g, 0.486 mmol) in dichloromethane (7 mL) were added trifluoroacetic acid (0.70 mL) and trifluoromethanesulfonic acid (0.35 mL) at 0° C. After stirring at 0° C. for 15 min, it was quenched with saturated sodium bicarbonate solution (100 mL), extracted with 20% methanol in dichloromethane (3×100 mL), combined organic layers dried over magnesium sulfate, filtered, concentrated, and the residue purified by flash column chromatography (Davisil silica, methanol/dichloromethane) to afford 3-((S)-6-(((1R,5S,6r)-3-oxabicyclo[3.1.0]hexan-6-yl)(methyl-d₃)amino)-4-aminopyrido[3,4-d]pyrimidin-8-yl)-2,4-dimethylphenol (63-B) (120 mg, 64%, 99.2% ee). Compounds 63-B and 63-C are atropisomers, and the stereochemistry may be the opposite of that depicted. ¹H NMR (400 MHz, DMSO-d₆): δ ppm 9.03 (s, 1H), 8.07 (s, 1H), 7.78 (bs, 2H), 7.10 (s, 1H), 6.85 (d, J=8.0 Hz, 1H), 6.73 (d, J=8.0 Hz, 1H), 4.17-4.14 (m, 2H), 3.68-3.66 (m, 2H), 2.42-2.38 (m, 1H), 1.98 (s, 2H), 1.72 (s, 3H), 1.63 (s, 3H); MS (ESI) 381.39 [M+H]⁺; HPLC: 99.20%.

### 3-((R)-6-(((1R,5S,6r)-3-oxabicyclo[3.1.0]hexan-6-yl)(methyl-d₃)amino)-4-aminopyrido[3,4-d]pyrimidin-8-yl)-2,4-dimethylphenol (63-C)

To a stirred solution of tert-butyl ((R)-6-(((1R,5S,6r)-3-oxabicyclo[3.1.0]hexan-6-yl)(methyl-d₃)amino)-8-(3-(benzyloxy)-2,6-dimethylphenyl)pyrido[3,4-d]pyrimidin-4-yl)(2,4-dimethoxybenzyl)carbamate (63-A2) (0.370 g, 0.513 mmol) in dichloromethane (7.4 mL), were added trifluoroacetic acid (0.74 mL) and trifluoromethanesulfonic acid (0.37 mL) at 0° C. After stirring at 0° C., it was quenched with saturated sodium bicarbonate solution (125 mL), extracted with 20% methanol in dichloromethane (3×125 mL), combined organic layers dried over magnesium sulfate, filtered, concentrated, and the residue purified by flash column chromatography (Davisil silica, methanol: dichloromethane) to afford 3-((R)-6-(((1R,5S,6r)-3-oxabicyclo[3.1.0]hexan-6-yl)(methyl-d₃)amino)-4-aminopyrido[3,4-d]pyrimidin-8-yl)-2,4-dimethylphenol (63-C) (140 mg, 71%, 98.2% ee). Compounds 63-B and 63-C are atropisomers, and the stereochemistry may be the opposite of that depicted. ¹H NMR (400 MHz, DMSO-d₆): δ ppm 9.03 (s, 1H), 8.07 (s, 1H), 7.78 (bs, 2H), 7.10 (s, 1H), 6.85 (d, J=8.0 Hz, 1H), 6.73 (d, J=8.0 Hz, 1H), 4.17-4.14 (m, 2H), 3.68-3.66 (m, 2H), 2.42-2.38 (m, 1H), 1.98 (m, 2H), 1.72 (s, 3H), 1.63 (s, 3H); MS (ESI) 381.37 [M+H]⁺; HPLC: 99.11%.

### Example 64

### (S)-3-(4-amino-6-((2-methoxyethyl)amino)pyrido[3,4-d]pyrimidin-8-yl)-2,4-dimethylphenol (64-B) and (R)-3-(4-amino-6-((2-methoxyethyl)amino)pyrido[3,4-d]pyrimidin-8-yl)-2,4-dimethylphenol (64-C)

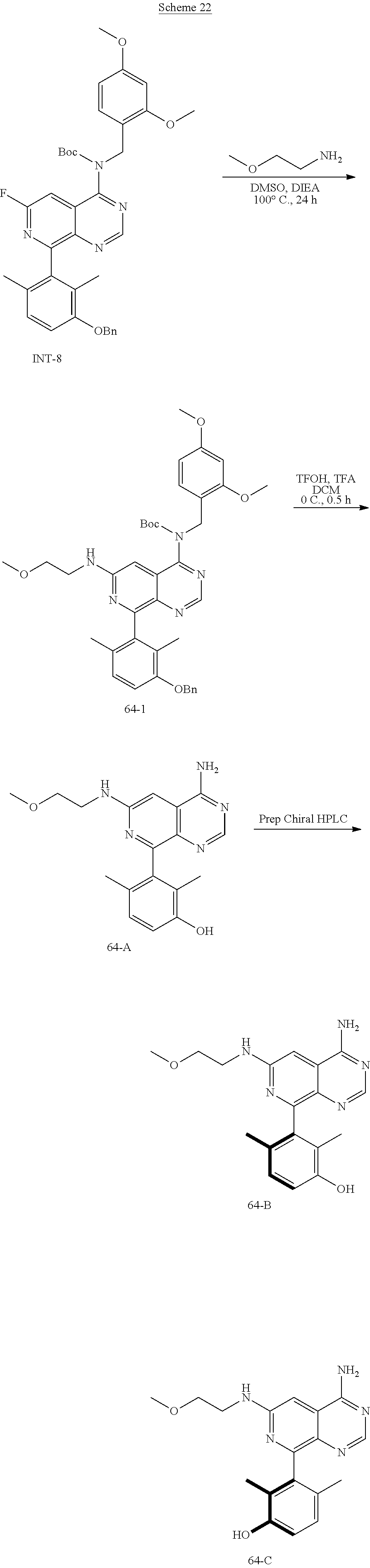

### tert-butyl (8-(3-(benzyloxy)-2,6-dimethylphenyl)-6-((2-methoxyethyl)amino)pyrido[3,4-d]pyrimidin-4-yl)(2,4-dimethoxybenzyl)carbamate (64-1)

To a stirred solution of tert-butyl (8-(3-(benzyloxy)-2,6-dimethylphenyl)-6-fluoropyrido[3,4-d]pyrimidin-4-yl)(2,4-dimethoxybenzyl)carbamate (INT-8) (1.00 g, 1.60 mmol) in DMSO (20 mL), were added DIEA (0.84 mL, 4.8 mmol) and 2-methoxyethan-1-amine (0.60 g, 8.0 mmol) at RT. After stirring at 100° C. for 24 h, the mixture was cooled down to RT, diluted with water (20 mL) and extracted with ethyl acetate (3×20 mL). The combined organic layers were washed with brine (3×), dried over anhydrous sodium sulfate, filtered, concentrated, and residue purified by flash column chromatography (SiO₂, 50% EtOAc/PET) to afford tert-butyl (8-(3-(benzyloxy)-2,6-dimethylphenyl)-6-((2-methoxyethyl)amino)pyrido[3,4-d]pyrimidin-4-yl)(2,4-dimethoxybenzyl)carbamate (64-1) (1.00 g, 91%). LCMS (MS, ESI): 680.65 [M+H]⁺.

### 3-(4-amino-6-((2-methoxyethyl)amino)pyrido[3,4-d]pyrimidin-8-yl)-2,4-dimethylphenol (64-A)

To a stirred mixture of tert-butyl (8-(3-(benzyloxy)-2,6-dimethylphenyl)-6-((2-methoxyethyl)amino)pyrido[3,4-d]pyrimidin-4-yl)(2,4-dimethoxybenzyl)carbamate (64-1) (1.00 g, 1.47 mmol) in DCM (20 mL), were added trifluoroacetic acid (2 mL) and trifluoromethanesulfonic acid (1 mL) at 0° C. After stirring at 0° C. for 0.5 h, the reaction mixture was quenched with sat. NaHCO₃ and extracted with ethyl acetate (3×30 mL). The combined organic layers were washed with water (30 mL), brine (30 mL), dried over sodium sulfate, filtered, concentrated, and residue purified by preparative-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 6% B to 36% B in 10 min; Wave Length: 254 nm/220 nm), followed by lyophilization, to give 3-(4-amino-6-((2-methoxyethyl)amino)pyrido[3,4-d]pyrimidin-8-yl)-2,4-dimethylphenol (64-A) (200 mg, 40%). LCMS (MS, ESI): 340.10 [M+H]⁺. ¹H NMR (400 MHz, DMSO-d₆) δ 9.01 (s, 1H), 8.01 (s, 1H), 7.67 (s, 2H), 6.89 (s, 1H), 6.86-6.84 (m, 1H), 6.73 (d, J=8.0 Hz, 1H), 6.51 (t, J=5.6 Hz, 1H), 3.55-3.52 (m, 2H), 3.41-3.34 (m, 2H), 3.29 (s, 3H), 1.73 (s, 3H), 1.65 (s, 3H).

### (S)-3-(4-amino-6-((2-methoxyethyl)amino)pyrido[3,4-d]pyrimidin-8-yl)-2,4-dimethylphenol (64-B) and (R)-3-(4-amino-6-((2-methoxyethyl)amino)pyrido[3,4-d]pyrimidin-8-yl)-2,4-dimethylphenol (64-C)

3-(4-Amino-6-((2-methoxyethyl)amino)pyrido[3,4-d]pyrimidin-8-yl)-2,4-dimethylphenol (64-A) (120 mg) was subjected to prep-chiral-HPLC (Column: CHIRALPAK AD-H, 20*250 mm, 5 μm; Mobile Phase A: Hex—HPLC, Mobile Phase B: MeOH:DCM=1: 1-HPLC; Flow rate: 20 mL/min; Gradient: isocratic 20; Wave Length: 254 nm/220 nm), to afford (S)-3-(4-amino-6-((2-methoxyethyl)amino)pyrido[3,4-d]pyrimidin-8-yl)-2,4-dimethylphenol (64-B) (89.2 mg, 45%), and (R)-3-(4-amino-6-((2-methoxyethyl)amino)pyrido[3,4-d]pyrimidin-8-yl)-2,4-dimethylphenol (64-C) (85.6 mg, 43%). Compounds 64-B and 64-C are atropisomers, and the shown stereochemistry may be the opposite of that depicted. 64-B: LCMS (MS, ESI): 340.15 [M+H]⁺. ¹H NMR (400 MHz, DMSO-d₆) δ 9.01 (s, 1H), 8.01 (s, 1H), 7.67 (brs, 2H), 6.89 (s, 1H), 6.84 (d, J=8.0 Hz, 1H), 6.73 (d, J=8.0 Hz, 1H), 6.51 (t, J=5.6 Hz, 1H), 3.55-3.52 (m, 2H), 3.41-3.34 (m, 2H), 3.29 (s, 3H), 1.73 (s, 3H), 1.65 (s, 3H). Chiral purity: 100% ee. 64-C: LCMS (MS, ESI): 340.15 [M+H]⁺. ¹H NMR (400 MHz, DMSO-d₆) δ 9.01 (s, 1H), 8.01 (s, 1H), 7.67 (brs, 2H), 6.89 (s, 1H), 6.84 (d, J=8.0 Hz, 1H), 6.73 (d, J=8.0 Hz, 1H), 6.51 (t, J=5.6 Hz, 1H), 3.55-3.52 (m, 2H), 3.41-3.34 (m, 2H), 3.29 (s, 3H), 1.73 (s, 3H), 1.65 (s, 3H). Chiral purity: 99.31% ee.

### Example 65

### (S)-3-(4-amino-6-(cyclopropylamino)pyrido[3,4-d]pyrimidin-8-yl)-2,4-dimethylphenol (65-B) and (R)-3-(4-amino-6-(cyclopropylamino)pyrido[3,4-d]pyrimidin-8-yl)-2,4-dimethylphenol (65-C)

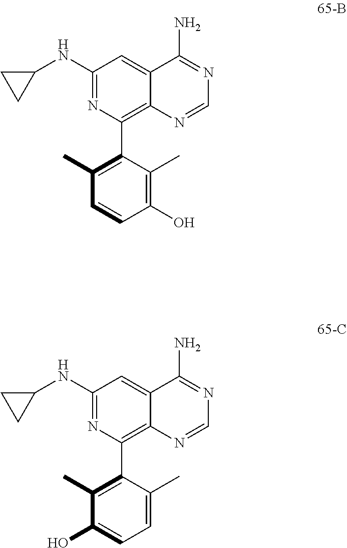

3-(4-amino-6-(cyclopropylamino)pyrido[3,4-d]pyrimidin-8-yl)-2,4-dimethylphenol (65-A) (200 mg) was prepared according to the procedure described in Example 64, and subjected to prep-SFC (Column: CHIRAL ART Cellulose-SJ 3*25 cm, 5 μm; Mobile Phase A: CO₂, Mobile Phase B: MeOH (0.3% 7M NH₃-MEOH); Flow rate: 85 mL/min; Gradient (B %): isocratic 16% B; Column Temperature (° C.): 35; Back Pressure(bar): 100; Wave Length: 220 nm; RT1(min)), to afford (S)-3-(4-amino-6-(cyclopropylamino)pyrido[3,4-d]pyrimidin-8-yl)-2,4-dimethylphenol (65-B) (48.6 mg, 24%), and (R)-3-(4-amino-6-(cyclopropylamino)pyrido[3,4-d]pyrimidin-8-yl)-2,4-dimethylphenol (65-C) (38.7 mg, 19%). Compounds 65-B and 65-C are atropisomers, and the shown stereochemistry may be the opposite of that depicted. 65-B: LCMS (MS, ESI): 322.20 [M+H]⁺. ¹H NMR (400 MHz, DMSO-d₆) δ 9.00 (s, 1H), 8.03 (s, 1H), 7.74 (brs, 2H), 7.05 (s, 1H), 6.98 (d, J=2.0 Hz, 1H), 6.84 (d, J=8.4 Hz, 1H), 6.72 (d, J=8.4 Hz, 1H), 2.49-2.42 (m, 1H), 1.72 (s, 3H), 1.63 (s, 3H), 0.90-0.72 (m, 2H), 0.61-0.42 (m, 2H). Chiral purity: 100% ee. 65-C: LCMS (MS, ESI): 322.15 [M+H]⁺. ¹H NMR (400 MHz, DMSO-d₆) δ 8.99 (s, 1H), 8.03 (s, 1H), 7.74 (brs, 2H), 7.05 (s, 1H), 6.98 (d, J=2.0 Hz, 1H), 6.84 (d, J=8.4 Hz, 1H), 6.72 (d, J=8.0 Hz, 1H), 2.49-2.42 (m, 1H), 1.72 (s, 3H), 1.63 (s, 3H), 0.92-0.78 (m, 2H), 0.62-0.48 (m, 2H). Chiral purity: 97.40% ee.

### Example 66

### (S)-3-{4-amino-6-[(2,2-difluoroethyl)amino]pyrido[3,4-d]pyrimidin-8-yl}-2,4-dimethylphenol (66-B) and (R)-3-{4-amino-6-[(2,2-difluoroethyl)amino]pyrido[3,4-d]pyrimidin-8-yl}-2,4-dimethylphenol (66-C)

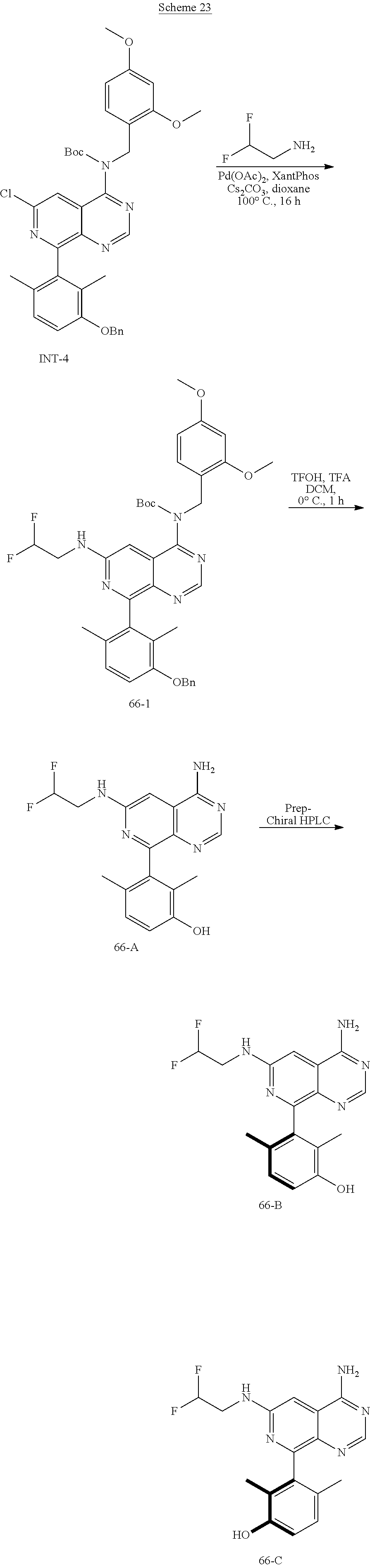

### tert-butyl N-{8-[3-(benzyloxy)-2,6-dimethylphenyl]-6-[(2,2-difluoroethyl)amino]pyrido[3,4-d]pyrimidin-4-yl}-N-[(2,4-dimethoxyphenyl)methyl]carbamate (66-1)

A mixture of tert-butyl (8-(3-(benzyloxy)-2,6-dimethylphenyl)-6-chloropyrido[3,4-d]pyrimidin-4-yl)(2,4-dimethoxybenzyl)carbamate (INT-4) (1.00 g, 1.56 mmol), 2,2-difluoroethanamine (0.38 g, 4.7 mmol), Pd(OAc)₂ (0.040 g, 0.16 mmol), XantPhos (0.18 g, 0.31 mmol) and Cs₂CO₃ (1.52 g, 4.66 mmol) in dioxane (20 mL) were degassed and backfilled with nitrogen five times. After stirring at 100° C. for 16 h, the reaction mixture was cooled down to RT, quenched with water (50 mL) and extracted with ethyl acetate (3×50 mL). The combined organic layers were washed with water (50 mL), brine (50 mL), dried over anhydrous sodium sulfate, filtered, concentrated, and residue purified by flash column chromatography (SiO₂, 30% EtOAc/PET), to afford tert-butyl N-{8-[3-(benzyloxy)-2,6-dimethylphenyl]-6-[(2,2-difluoroethyl)amino]pyrido[3,4-d]pyrimidin-4-yl}-N-[(2,4-dimethoxyphenyl)methyl]carbamate (66-1) (0.70 g, 65%). LCMS (MS, ESI): 686.30 [M+H]⁺.

### 3-{4-amino-6-[(2,2-difluoroethyl)amino]pyrido[3,4-d]pyrimidin-8-yl}-2,4-dimethylphenol (66-A)

To a stirred mixture of tert-butyl N-{8-[3-(benzyloxy)-2,6-dimethylphenyl]-6-[(2,2-difluoroethyl)amino]pyrido[3,4-d]pyrimidin-4-yl}-N-[(2,4-dimethoxyphenyl)methyl]carbamate (66-1) (0.600 g, 0.875 mmol) in DCM (12 mL), were added trifluoroacetic acid (1.2 mL) and trifluoromethanesulfonic acid (0.6 mL) dropwise at 0° C. After stirring at 0° C. for 1 h, the reaction mixture was basified to pH 8 with aq. saturated NaHCO₃ and extracted with ethyl acetate (3×30 mL). The combined organic layers were dried over anhydrous sodium sulfate, filtered, concentrated, and residue purified by preparative-HPLC (Column: WelFlash C18, Regular C18 20-40 μm, 120 g; Mobile Phase A: Water (10 mmol/L NH₄HCO₃+0.05% NH₃), Mobile Phase B: ACN; Flow rate: 60 mL/min; Gradient (B %): 10% B to 40% B in 30 min; Wave Length: 254 nm/220 nm), to afford 3-{4-amino-6-[(2,2-difluoroethyl)amino]pyrido[3,4-d]pyrimidin-8-yl}-2,4-dimethylphenol (66-A) (140 mg, 46%). LCMS (MS, ESI): 346.10 [M+H]⁺. ¹H NMR (400 MHz, DMSO-d₆) δ 9.03 (s, 1H), 8.04 (s, 1H), 7.73 (s, 2H), 7.04 (s, 1H), 6.96 (t, J=6.4 Hz, 1H), 6.85 (d, J=8.0 Hz, 1H), 6.73 (d, J=8.0 Hz, 1H), 6.27-5.99 (m, 1H), 3.72-3.62 (m, 2H), 1.72 (s, 3H), 1.64 (s, 3H).

### (S)-3-{4-amino-6-[(2,2-difluoroethyl)amino]pyrido[3,4-d]pyrimidin-8-yl}-2,4-dimethylphenol (66-B) and (R)-3-{4-amino-6-[(2,2-difluoroethyl)amino]pyrido[3,4-d]pyrimidin-8-yl}-2,4-dimethylphenol (66-C)

3-{4-Amino-6-[(2,2-difluoroethyl)amino]pyrido[3,4-d]pyrimidin-8-yl}-2,4-dimethylphenol (66-A) (140 mg, 0.371 mmol) was subjected to prep-chiral-HPLC (Column: CHIRALPAK IJ 250*20 mm, 5 μm; Mobile Phase A: Hex (0.5% 2M NH₃-MeOH), Mobile Phase B: MeOH:DCM=1:1-HPLC; Flow rate: 20 mL/min; Gradient (B %): isocratic 20; Wave Length: 254/220 nm), to afford (S)-3-{4-amino-6-[(2,2-difluoroethyl)amino]pyrido[3,4-d]pyrimidin-8-yl}-2,4-dimethylphenol (66-B) (43.5 mg, 31%), and (R)-3-{4-amino-6-[(2,2-difluoroethyl)amino]pyrido[3,4-d]pyrimidin-8-yl}-2,4-dimethylphenol (66-C) (42.8 mg, 30%). Compounds 66-B and 66-C are atropisomers, and the shown stereochemistry may be the opposite of that depicted. 66-B: LCMS (MS, ESI): 346.05 [M+H]⁺. ¹H NMR (400 MHz, DMSO-d₆) δ 9.04 (brs, 1H), 8.03 (s, 1H), 7.72 (s, 2H), 7.03 (s, 1H), 6.95 (t, J=6.0 Hz, 1H), 6.85 (d, J=8.4 Hz, 1H), 6.73 (d, J=8.0 Hz, 1H), 6.27-5.97 (m, 1H), 3.72-3.62 (m, 2H), 1.72 (s, 3H), 1.63 (s, 3H). ¹⁹F NMR (376 MHz, DMSO-d₆) δ −121.50. Chiral purity: 99.75% ee. 66-C: LCMS (MS, ESI): 346.00 [M+H]⁺. ¹H NMR (400 MHz, DMSO-d₆) δ 9.03 (s, 1H), 8.03 (s, 1H), 7.72 (s, 2H), 7.03 (s, 1H), 6.95 (t, J=6.4 Hz, 1H), 6.85 (d, J=8.4 Hz, 1H), 6.73 (d, J=8.0 Hz, 1H), 6.26-5.98 (m, 1H), 3.72-3.62 (m, 2H), 1.72 (s, 3H), 1.63 (s, 3H). ¹⁹F NMR (376 MHz, DMSO-d₆) δ −121.50. Chiral purity: 99.40% ee.

### Example 67

### (S)-3-(4-amino-6-((2,2,2-trifluoroethyl)amino)pyrido[3,4-d]pyrimidin-8-yl)-2,4-dimethylphenol (67-B) and (R)-3-(4-amino-6-((2,2,2-trifluoroethyl)amino)pyrido[3,4-d]pyrimidin-8-yl)-2,4-dimethylphenol (67-C)

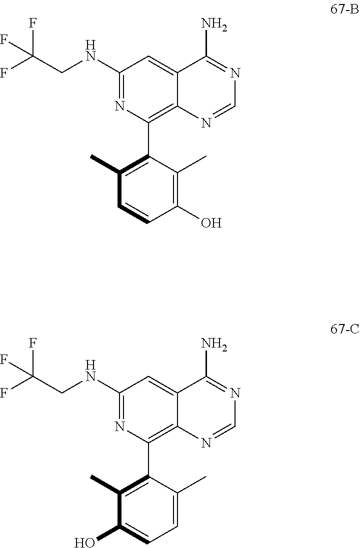

3-(4-amino-6-((2,2,2-trifluoroethyl)amino)pyrido[3,4-d]pyrimidin-8-yl)-2,4-dimethylphenol (67-A) (200 mg) was prepared according to the procedure described in Example 66, and subjected to prep-chiral-HPLC (CHIRALPAK IJ, 20*250 mm, 5 μm; Mobile Phase A: Hex(0.5% 2M NH₃-MeOH), Mobile Phase B: MeOH:DCM=1:1; Flow rate: 20 mL/min; Gradient: isocratic 15; Wave Length: 254 nm/220 nm), to afford (S)-3-(4-amino-6-((2,2,2-trifluoroethyl)amino)pyrido[3,4-d]pyrimidin-8-yl)-2,4-dimethylphenol (67-B) (75.5 mg, 38%), and (R)-3-(4-amino-6-((2,2,2-trifluoroethyl)amino)pyrido[3,4-d]pyrimidin-8-yl)-2,4-dimethylphenol (67-C) (72.7 mg, 36%). Compounds 67-B and 67-C are atropisomers, and the shown stereochemistry may be the opposite of that depicted. 67-B: LCMS (MS, ESI): 364.10 [M+H]⁺. ¹H NMR (400 MHz, DMSO-d₆) δ 9.04 (s, 1H), 8.06 (s, 1H), 7.75 (s, 2H), 7.23 (t, J=6.4 Hz, 1H), 7.11 (s, 1H), 6.86 (d, J=8.4 Hz, 1H), 6.74 (d, J=8.0 Hz, 1H), 4.15-4.06 (m, 2H), 1.71 (s, 3H), 1.63 (s, 3H). ¹⁹F NMR (376 MHz, DMSO-d₆) δ −70.78. Chiral purity: 100% ee. 67-C: LCMS (MS, ESI): 364.20 [M+H]⁺. ¹H NMR (400 MHz, DMSO-d₆) δ 9.04 (s, 1H), 8.06 (s, 1H), 7.75 (s, 2H), 7.23 (t, J=6.8 Hz, 1H), 7.11 (s, 1H), 6.86 (d, J=8.0 Hz, 1H), 6.74 (d, J=8.4 Hz, 1H), 4.15-4.06 (m, 2H), 1.71 (s, 3H), 1.63 (s, 3H). ¹⁹F NMR (376 MHz, DMSO-d₆) δ −70.78. Chiral purity: 99.49% ee.

### Example 68

### (S)-3-(4-amino-6-((methyl-d₃)amino)pyrido[3,4-d]pyrimidin-8-yl)-2,4-dimethylphenol (68-B) and (R)-3-(4-amino-6-((methyl-d₃)amino)pyrido[3,4-d]pyrimidin-8-yl)-2,4-dimethylphenol (68-C)

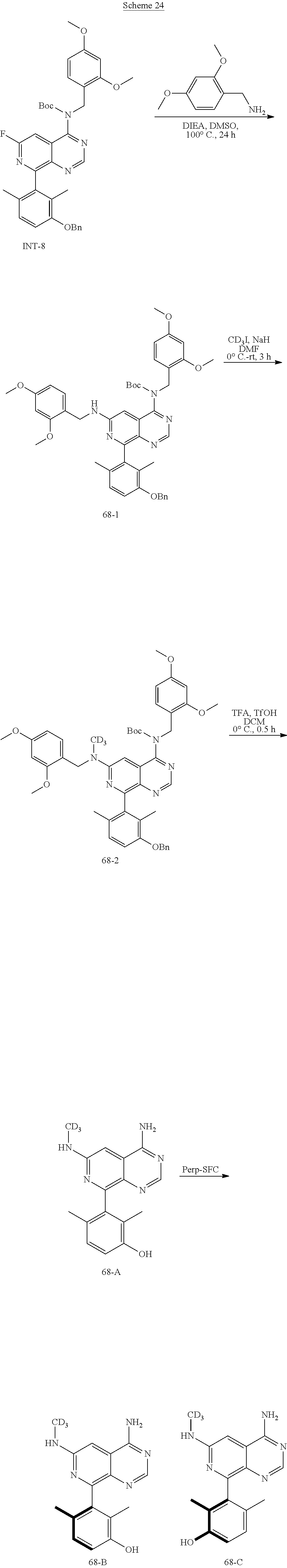

### tert-butyl (8-(3-(benzyloxy)-2,6-dimethylphenyl)-6-((2,4-dimethoxybenzyl)amino)pyrido[3,4-d]pyrimidin-4-yl)(2,4-dimethoxybenzyl)carbamate (68-1)

To a stirred mixture of tert-butyl (8-(3-(benzyloxy)-2,6-dimethylphenyl)-6-fluoropyrido[3,4-d]pyrimidin-4-yl)(2,4-dimethoxybenzyl)carbamate (INT-8) (1.00 g, 1.60 mmol) in DMSO (16 mL), were added DIEA (0.83 mL, 4.8 mmol) and 1-(2,4-dimethoxyphenyl)methanamine (1.34 g, 8.00 mmol) at RT. After stirring at 100° C. for 24 h, the reaction mixture was cooled down to RT, quenched with water (30 mL) and extracted with ethyl acetate (3×30 mL). The combined organic layers were washed with water (30 mL), brine (30 mL), dried over anhydrous sodium sulfate, filtered, concentrated, and residue purified by flash column chromatography (SiO₂, 23% EtOAc/PET), to afford tert-butyl (8-(3-(benzyloxy)-2,6-dimethylphenyl)-6-((2,4-dimethoxybenzyl)amino)pyrido[3,4-d]pyrimidin-4-yl)(2,4-dimethoxybenzyl)carbamate (68-1) (1.00 g, 84%). LCMS (MS, ESI): 772.30 [M+H]⁺.

### tert-butyl (8-(3-(benzyloxy)-2,6-dimethylphenyl)-6-((2,4-dimethoxybenzyl)(methyl-d₃)amino)pyrido[3,4-d]pyrimidin-4-yl)(2,4-dimethoxybenzyl)carbamate (68-2)

To a stirred mixture of tert-butyl (8-(3-(benzyloxy)-2,6-dimethylphenyl)-6-((2,4-dimethoxybenzyl)amino)pyrido[3,4-d]pyrimidin-4-yl)(2,4-dimethoxybenzyl)carbamate (68-1) (0.950 g, 1.28 mmol) in DMF (13 mL), was added NaH (0.100 g, 2.57 mmol, 60% purity) at 0° C. After stirring for 30 min at RT, it was cooled to 0° C. and added CD₃I (0.56 g, 3.8 mmol), stirred at RT for 2.5 h, quenched with water (20 mL) and extracted with ethyl acetate (3×30 mL). The combined organic layers were washed with water (30 mL), brine (30 mL), dried over anhydrous sodium sulfate, filtered, concentrated, and residue purified by reverse-phase flash chromatography (mobile phase, MeCN in Water, 10% to 75% gradient in 40 min; detector, UV 254 nm), to afford tert-butyl (8-(3-(benzyloxy)-2,6-dimethylphenyl)-6-((2,4-dimethoxybenzyl)(methyl-d₃)amino)pyrido[3,4-d]pyrimidin-4-yl)(2,4-dimethoxybenzyl)carbamate (68-2) (0.56 g, 57%). LCMS (MS, ESI): 789.35 [M+H]⁺.

### 3-(4-amino-6-((methyl-d₃)amino)pyrido[3,4-d]pyrimidin-8-yl)-2,4-dimethylphenol (68-A)

To a stirred mixture of tert-butyl (8-(3-(benzyloxy)-2,6-dimethylphenyl)-6-((2,4-dimethoxybenzyl)(methyl-d₃)amino)pyrido[3,4-d]pyrimidin-4-yl)(2,4-dimethoxybenzyl)carbamate (68-2) (0.500 g, 0.661 mmol) in DCM (10 mL), were added TFA (1.0 mL) and TfOH (0.5 mL) at 0° C. After stirred at 0° C. for 30 min, the reaction mixture was basified to pH 8 with aq. saturated NaHCO₃ and extracted with ethyl acetate (3×20 mL). Combined organic layers were washed with water (20 mL), brine (20 mL), dried over anhydrous sodium sulfate, filtered, concentrated, and residue purified by preparative-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 4% B to 34% B in 10 min; Wave Length: 254 nm/220 nm), followed by lyophilization, to give 3-(4-amino-6-((methyl-d₃)amino)pyrido[3,4-d]pyrimidin-8-yl)-2,4-dimethylphenol (68-A) (90 mg, 45%). LCMS (MS, ESI): 299.35 [M+H]⁺. ¹H NMR (400 MHz, DMSO-d₆) δ 9.00 (s, 1H), 8.00 (s, 1H), 7.67 (brs, 2H), 6.84 (d, J=8.0 Hz, 1H), 6.78 (s, 1H), 6.72 (d, J=8.0 Hz, 1H), 6.54 (s, 1H), 1.72 (s, 3H), 1.64 (s, 3H).

### (S)-3-(4-amino-6-((methyl-d₃)amino)pyrido[3,4-d]pyrimidin-8-yl)-2,4-dimethylphenol (68-B) and (R)-3-(4-amino-6-((methyl-d₃)amino)pyrido[3,4-d]pyrimidin-8-yl)-2,4-dimethylphenol (68-C)

3-(4-Amino-6-((methyl-d₃)amino)pyrido[3,4-d]pyrimidin-8-yl)-2,4-dimethylphenol (68-A) (90 mg) was subjected to prep-SFC (Column: CHIRAL ART Cellulose-SJ, 3*25 cm, 5 μm; Mobile Phase A: CO₂, Mobile Phase B: MeOH (0.1% 2M NH₃-MeOH); Flow rate: 85 mL/min; Gradient (B %): isocratic 24% B; Column Temperature(° C.): 35; Back Pressure(bar): 100; Wave Length: 220 nm), to afford (S)-3-(4-amino-6-((methyl-d₃)amino)pyrido[3,4-d]pyrimidin-8-yl)-2,4-dimethylphenol (68-B) (29.1 mg, 32%), and (R)-3-(4-amino-6-((methyl-d₃)amino)pyrido[3,4-d]pyrimidin-8-yl)-2,4-dimethylphenol (68-C) (26.1 mg, 29%). Compounds 68-B and 68-C are atropisomers, and the shown stereochemistry may be the opposite of that depicted. 68-B: LCMS (MS, ESI): 299.30 [M+H]⁺. ¹H NMR (400 MHz, DMSO-d₆) δ 9.00 (brs, 1H), 8.01 (s, 1H), 7.67 (brs, 2H), 6.84 (d, J=8.4 Hz, 1H), 6.79 (s, 1H), 6.72 (d, J=8.0 Hz, 1H), 6.54 (s, 1H), 1.72 (s, 3H), 1.64 (s, 3H). Chiral purity: 100% ee. 68-C: LCMS (MS, ESI): 299.25 [M+H]⁺. ¹H NMR (400 MHz, DMSO-d₆) δ 9.00 (s, 1H), 8.00 (s, 1H), 7.67 (brs, 2H), 6.84 (d, J=8.4 Hz, 1H), 6.78 (s, 1H), 6.72 (d, J=8.0 Hz, 1H), 6.54 (s, 1H), 1.72 (s, 3H), 1.64 (s, 3H). Chiral purity: 100% ee.

### Example 69

### (S)-3-[4-amino-6-(isopropylamino)pyrido[3,4-d]pyrimidin-8-yl]-2,4-dimethylphenol (69-B) and (R)-3-[4-amino-6-(isopropylamino)pyrido[3,4-d]pyrimidin-8-yl]-2,4-dimethylphenol (69-C)

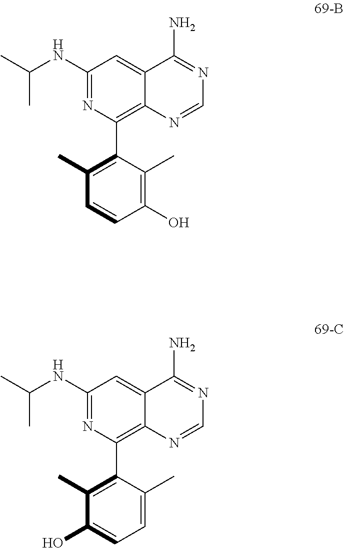

3-[4-Amino-6-(isopropylamino)pyrido[3,4-d]pyrimidin-8-yl]-2,4-dimethylphenol (69-A) (104 mg) was prepared according to the procedure described in Example 64, and subjected to prep-chiral-HPLC (CHIRALPAK IJ 250*20 mm, 5 μm; Mobile Phase A: Hex (0.5% 2M NH₃-MeOH), Mobile Phase B: EtOH; Flow rate: 20 mL/min; Gradient (B %): isocratic 15; Wave Length: 254/220 nm), to afford (S)-3-[4-amino-6-(isopropylamino)pyrido[3,4-d]pyrimidin-8-yl]-2,4-dimethylphenol (69-B) (34.0 mg, 23%), and (R)-3-[4-amino-6-(isopropylamino)pyrido[3,4-d]pyrimidin-8-yl]-2,4-dimethylphenol (69-C) (34.2 mg, 23%). Compounds 69-B and 69-C are atropisomers, and the shown stereochemistry may be the opposite of that depicted. 69-B: LCMS (MS, ESI): 324.05 [M+H]⁺. ¹H NMR (400 MHz, DMSO-d₆) δ 8.99 (s, 1H), 7.98 (s, 1H), 7.65 (brs, 2H), 6.85-6.81 (m, 2H), 6.71 (d, J=8.0 Hz, 1H), 6.39 (d, J=8.0 Hz, 1H), 3.85-3.78 (m, 1H), 1.72 (s, 3H), 1.64 (s, 3H), 1.19 (d, J=6.4 Hz, 6H). Chiral purity: 99.92% ee. 69-C: LCMS (MS, ESI): 324.10 [M+H]⁺. ¹H NMR (400 MHz, DMSO-d₆) δ 8.99 (s, 1H), 7.98 (s, 1H), 7.64 (brs, 2H), 6.85-6.81 (m, 2H), 6.71 (d, J=8.4 Hz, 1H), 6.39 (d, J=8.4 Hz, 1H), 3.83-3.78 (m, 1H), 1.72 (s, 3H), 1.64 (s, 3H), 1.19 (d, J=6.4 Hz, 6H). Chiral purity: 99.25% ee.

### Example 70

### (S)-3-(4-amino-6-((cyclopropylmethyl)amino)pyrido[3,4-d]pyrimidin-8-yl)-2,4-dimethylphenol (70-B) and (R)-3-(4-amino-6-((cyclopropylmethyl)amino)pyrido[3,4-d]pyrimidin-8-yl)-2,4-dimethylphenol (70-C)

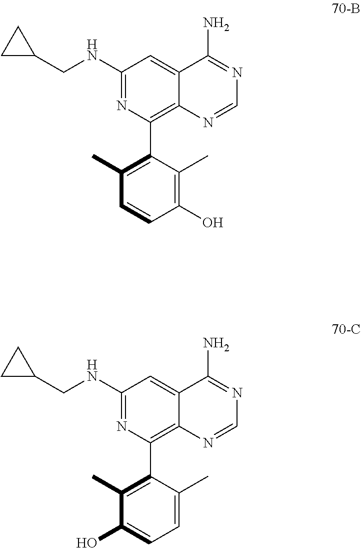

3-(4-Amino-6-((cyclopropylmethyl)amino)pyrido[3,4-d]pyrimidin-8-yl)-2,4-dimethylphenol (70-A) (193 mg) was prepared according to the procedure described in Example 64, and subjected to prep-SFC (Column: Lux 5 m Cellulose-3 30*250 mm, 5.0 μm; Mobile Phase A: CO₂, Mobile Phase B: MeOH (1%-2M-NH₃-MeOH); Flow rate: 90 mL/min; Gradient (B %): isocratic 20% B; Column Temperature(° C.): 35; Back Pressure(bar): 100; Wave Length: 220 nm), to afford (S)-3-(4-amino-6-((cyclopropylmethyl)amino)pyrido[3,4-d]pyrimidin-8-yl)-2,4-dimethylphenol (70-B) (83.2 mg, 43%), and (R)-3-(4-amino-6-((cyclopropylmethyl)amino)pyrido[3,4-d]pyrimidin-8-yl)-2,4-dimethylphenol (70-C) (81.5 mg, 42%). Compounds 70-B and 70-C are atropisomers, and the shown stereochemistry may be the opposite of that depicted. 70-B: LCMS (MS, ESI): 336.30 [M+H]⁺. ¹H NMR (400 MHz, DMSO-d₆) δ 9.01 (s, 1H), 7.99 (s, 1H), 7.65 (brs, 2H), 6.92-6.79 (m, 2H), 6.72 (d, J=8.0 Hz, 1H), 6.64 (t, J=6.0 Hz, 1H), 3.10 (t, J=6.4 Hz, 2H), 1.73 (s, 3H), 1.65 (s, 3H), 1.21-1.09 (m, 1H), 0.52-0.40 (m, 2H), 0.28-0.19 (m, 2H). Chiral purity: 100% ee. 70-C: LCMS (MS, ESI): 336.35 [M+H]⁺. ¹H NMR (400 MHz, DMSO-d₆) δ 9.01 (s, 1H), 7.99 (s, 1H), 7.65 (brs, 2H), 6.96-6.81 (m, 2H), 6.72 (d, J=8.0 Hz, 1H), 6.64 (t, J=6.0 Hz, 1H), 3.10 (t, J=6.4 Hz, 2H), 1.73 (s, 3H), 1.65 (s, 3H), 1.20-1.09 (m, 1H), 0.53-0.40 (m, 2H), 0.32-0.19 (m, 2H). Chiral purity: 100% ee.

### Example 71

### (R)-3-(4-amino-6-(3,6-dihydro-2H-pyran-4-yl)pyrido[3,2-d]pyrimidin-8-yl)-2,4-dimethylphenol (71-B) and (S)-3-(4-amino-6-(3,6-dihydro-2H-pyran-4-yl)pyrido[3,2-d]pyrimidin-8-yl)-2,4-dimethylphenol (71-C)

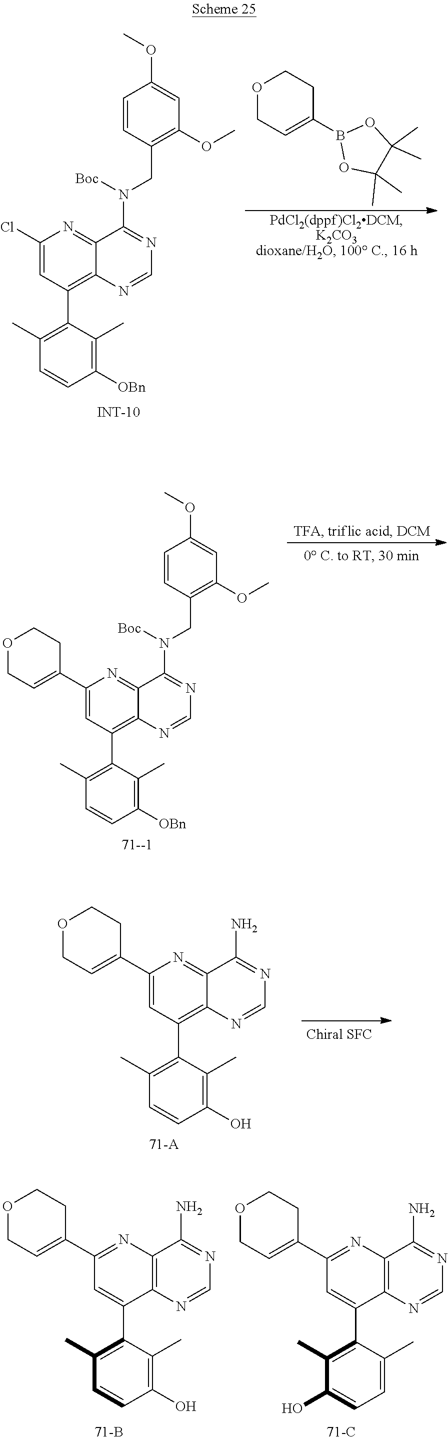

### tert-butyl (8-(3-(benzyloxy)-2,6-dimethylphenyl)-6-(3,6-dihydro-2H-pyran-4-yl)pyrido[3,2-d]pyrimidin-4-yl)(2,4-dimethoxybenzyl)carbamate (71-1)

To a stirred solution of tert-butyl (8-(3-(benzyloxy)-2,6-dimethylphenyl)-6-chloropyrido[3,2-d]pyrimidin-4-yl)(2,4-dimethoxybenzyl)carbamate (INT-10) (0.4 g, 0.7 mmol) in dioxane/water (9.0 mL, 1: 1) were added 2-(3,6-dihydro-2H-pyran-4-yl)-4,4,5,5-tetramethyl-1,3,2-dioxaborolane (1.5 g, 7.0 mmol) and K₂CO₃ (0.3 g, 2.1 mmol). The resulting mixture was purged with nitrogen for 15 min followed by the addition of [1,1′-bis(diphenylphosphino)ferrocene]dichloropalladium(II) DCM complex (0.06 g, 0.07 mmol). The resulting reaction mixture was stirred at 100° C. for 16 h before being diluted with EtOAc (40 mL), washed with water (2×15 mL) and brine (2×15 mL). The organic layers were dried over Na₂SO₄, filtered, and concentrated. The residue was purified by column chromatography (SiO₂, EtOAc/pet ether) to afford tert-butyl (8-(3-(benzyloxy)-2,6-dimethylphenyl)-6-(3,6-dihydro-2H-pyran-4-yl)pyrido[3,2-d]pyrimidin-4-yl)(2,4-dimethoxybenzyl)carbamate (71-C) (0.39 g, 80%). ¹H NMR (400 MHz, CDCl₃) δ ppm 9.05 (s, 1H), 7.75 (s, 1H), 7.64 (d, J=8.4 Hz, 1H), 7.46 (d, J=7.6 Hz, 2H), 7.40-7.31 (m, 3H), 7.13 (d, J=8.4 Hz, 1H), 6.95 (d, J=8.4 Hz, 1H), 6.73 (s, 1H), 6.41 (dd, J=8.4, 2.4 Hz, 1H), 6.29 (d, J=2.0 Hz, 1H), 5.20 (s, 2H), 5.11 (s, 2H), 4.39 (d, J=2.4 Hz, 2H), 3.90 (t, J=5.2 Hz, 2H), 3.76 (s, 3H), 3.40 (s, 3H), 2.69 (s, 2H), 1.87 (s, 3H), 1.86 (s, 3H), 1.25 (s, 9H). MS (ESI) m/z: 689.36 [M+H]⁺.

### 3-(4-amino-6-(3,6-dihydro-2H-pyran-4-yl)pyrido[3,2-d]pyrimidin-8-yl)-2,4-dimethylphenol (71-A)

At 0° C., to a stirred solution of tert-butyl (8-(3-(benzyloxy)-2,6-dimethylphenyl)-6-(3,6-dihydro-2H-pyran-4-yl)pyrido[3,2-d]pyrimidin-4-yl)(2,4-dimethoxybenzyl)carbamate (71-C) (0.38 g, 0.55 mmol) in DCM (15.2 mL) were added TFA (3.8 mL) and triflic acid (1.9 mL). The resulting reaction mixture was stirred at rt for 30 min before being diluted with EtOAc (40 mL), washed with saturated NaHCO₃ (2×15 mL) and brine (2×15 mL). The organic layers were dried over MgSO₄, filtered, and concentrated. The residue was purified by achiral SFC purification [(DCPAK P4VP, 30×250 mm column, CO₂/27% MeOH with 0.1% methanolic NH₃ (7N)] to afford 3-(4-amino-6-(3,6-dihydro-2H-pyran-4-yl)pyrido[3,2-d]pyrimidin-8-yl)-2,4-dimethylphenol (71-A) (0.06 g, 31%). MS (ESI) m/z: 349.23 [M+H]⁺.

### Isolation of (R)-3-(4-amino-6-(3,6-dihydro-2H-pyran-4-yl)pyrido[3,2-d]pyrimidin-8-yl)-2,4-dimethylphenol (71-B) & (S)-3-(4-amino-6-(3,6-dihydro-2H-pyran-4-yl)pyrido[3,2-d]pyrimidin-8-yl)-2,4-dimethylphenol (71-C)

The racemic mixture of 3-(4-amino-6-(3,6-dihydro-2H-pyran-4-yl)pyrido[3,2-d]pyrimidin-8-yl)-2,4-dimethylphenol (71-A) (0.06 g) was subjected to chiral SFC purification [CHIRALCEL OX—H, 30×250 mm column, CO₂/40% MeOH with 0.1% methanolic NH₃ (7N)] to afford (R)-3-(4-Amino-6-(3,6-dihydro-2H-pyran-4-yl)pyrido[3,2-d]pyrimidin-8-yl)-2,4-dimethylphenol (71-B) (24 mg, 40%) and (S)-3-(4-amino-6-(3,6-dihydro-2H-pyran-4-yl)pyrido[3,2-d]pyrimidin-8-yl)-2,4-dimethylphenol (71-C) (26 mg, 43%). 71-B: ¹H NMR (400 MHz, DMSO-d₆): δ ppm 9.15 (s, 1H), 8.24 (s, 1H), 7.90 (br s, 2H), 7.80 (s, 1H), 7.03 (s, 1H), 6.91 (d, J=8.4 Hz, 1H), 6.77 (d, J=8.0 Hz, 1H), 4.30 (d, J=2.8 Hz, 2H), 3.86 (t, J=5.2 Hz, 2H), 2.77 (s, 2H), 1.74 (s, 3H), 1.67 (s, 3H); MS (ESI) m/z: 349.36 [M+H]⁺; Chiral purity 99.9% (Lux Cellulose-4, 4.6×150 mm column, CO₂/30% MeOH with 0.5% DEA). 71-C: ¹H NMR (400 MHz, DMSO-d₆): δ ppm 9.15 (s, 1H), 8.24 (s, 1H), 7.90 (br. s, 2H), 7.80 (s, 1H), 7.03 (br. s, 1H), 6.91 (d, J=8.4 Hz, 1H), 6.77 (d, J=8.0 Hz, 1H), 4.30 (d, J=2.8 Hz, 2H), 3.86 (t, J=5.4 Hz, 2H), 2.80-2.72 (m, 2H), 1.74 (s, 3H), 1.67 s, 3H); MS (ESI) m/z: 349.35 [M+H]⁺; Chiral purity 99.7% (Lux Cellulose-4, 4.6×150 mm column, CO₂/30% MeOH with 0.5% DEA). Compounds (71-B) and (71-C) are atropisomers, and the stereochemistry may be the opposite of that depicted.

### Example 72

### (R)-3-(4-amino-6-(cyclopropylamino)pyrido[3,2-d]pyrimidin-8-yl)-2,4-dimethylphenol (72-B) and (S)-3-(4-amino-6-(cyclopropylamino)pyrido[3,2-d]pyrimidin-8-yl)-2,4-dimethylphenol (72-C)

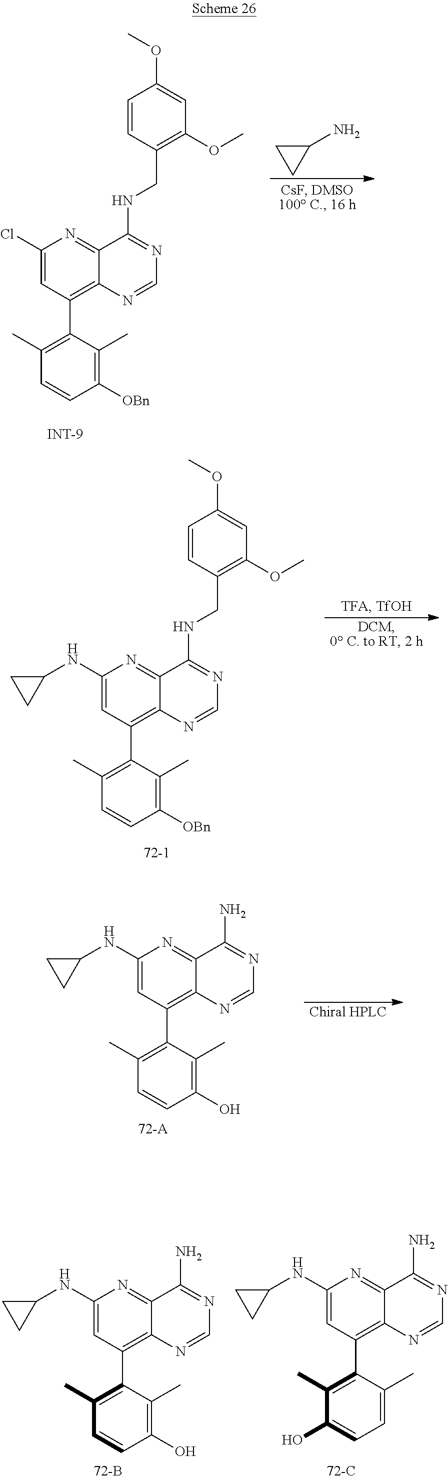

### 8-(3-(benzyloxy)-2,6-dimethylphenyl)-N⁶-cyclopropyl-N⁴-(2,4-dimethoxybenzyl)pyrido[3,2-d]pyrimidine-4,6-diamine (72-1)

At rt, to a stirred solution of 8-(3-(benzyloxy)-2,6-dimethylphenyl)-6-chloro-N-(2,4-dimethoxybenzyl)pyrido[3,2-d]pyrimidin-4-amine (INT-9) (0.7 g, 1.3 mmol) and CsF (0.6 g, 3.9 mmol) in DMSO (7 mL) was added cyclopropylamine (0.9 mL, 12.9 mmol). The resulting reaction mixture was stirred at 100° C. for 16 h, cooled to rt and diluted with water (20 mL). The mixture was extracted with EtOAc (3×20 mL). The combined organic layers were washed with brine (2×10 mL), dried over Na₂SO₄, filtered, and concentrated. The residue was purified by column chromatography (SiO₂, EtOAc/pet ether) to afford 8-(3-(benzyloxy)-2,6-dimethylphenyl)-N⁶-cyclopropyl-N⁴-(2,4-dimethoxybenzyl)pyrido[3,2-d]pyrimidine-4,6-diamine (72-1) (0.60 g, 82%). MS (ESI) m/z: 562.35 [M+H]⁺.

### 3-(4-amino-6-(cyclopropylamino)pyrido[3,2-d]pyrimidin-8-yl)-2,4-dimethylphenol (72-A)

At 0° C., to a stirred solution of 8-(3-(benzyloxy)-2,6-dimethylphenyl)-N⁶-cyclopropyl-N⁴-(2,4-dimethoxybenzyl)pyrido[3,2-d]pyrimidine-4,6-diamine (72-1) (0.5 g, 0.9 mmol) in DCM (20 mL) were added TFA (5 mL) and triflic acid (1.5 mL). The resulting reaction mixture was stirred at rt for 2 h, diluted with EtOAc (50 mL) and washed with saturated NaHCO₃ solution (2×15 mL). The organic layers were dried over Na₂SO₄, filtered, and concentrated. The residue was purified by preparative-HPLC (XBridge Prep OBD C18, 30×150 mm column, ACN/water with 10 mmol NH₄HCO₃) to afford 3-(4-amino-6-(cyclopropylamino)pyrido[3,2-d]pyrimidin-8-yl)-2,4-dimethylphenol (72-A) (0.11 g, 32%). ¹H NMR (400 MHz, DMSO-d₆): δ ppm 9.12 (s, 1H), 8.03 (s, 1H), 7.65-7.40 (m, 1H), 7.39-7.34 (m, 1H), 7.02-6.80 (m, 2H), 6.78-6.70 (m, 2H), 2.95-2.80 (m, 1H), 1.78 (s, 3H), 1.70 (s, 3H), 0.84-0.73 (m, 2H), 0.55-0.43 (m, 2H); MS (ESI) m/z: 322.0 [M+H]⁺.

### Isolation of (R)-3-(4-amino-6-(cyclopropylamino)pyrido[3,2-d]pyrimidin-8-yl)-2,4-dimethylphenol (72-B) and (S)-3-(4-amino-6-(cyclopropylamino)pyrido[3,2-d]pyrimidin-8-yl)-2,4-dimethylphenol (72-C)

The racemic mixture of 3-(4-amino-6-(cyclopropylamino)pyrido[3,2-d]pyrimidin-8-yl)-2,4-dimethylphenol (72-A) (0.11 g) was subjected to Prep-chiral HPLC purification [CHIRAL ART Cellulose-SB, 20×250 mm column, hexane with 0.3% IPA-amine/15% MeOH/DCM (1:1)] to afford (R)-3-(4-amino-6-(cyclopropylamino)pyrido[3,2-d]pyrimidin-8-yl)-2,4-dimethylphenol (72-B) (29.3 mg, 26%) and (S)-3-(4-amino-6-(cyclopropylamino)pyrido[3,2-d]pyrimidin-8-yl)-2,4-dimethylphenol (72-C) (25.9 mg, 23%). 72-B: ¹H NMR (400 MHz, DMSO-d₆): δ ppm 9.12 (s, 1H), 8.03 (s, 1H), 7.48-7.28 (m, 2H), 6.95-6.89 (m, 1H), 6.78-6.70 (m, 2H), 2.98-2.78 (m, 1H), 1.78 (s, 3H), 1.70 (s, 3H), 0.84-0.73 (m, 2H), 0.55-0.43 (m, 2H); MS (ESI) m/z: 322.15 [M+H]⁺; Chiral purity 98.9% (CHIRAL ART Cellulose-SB, 4.6×50 mm column, hexane with 0.3% IPA-amine/15% MeOH/DCM (1:1)). 72-C: ¹H NMR (400 MHz, DMSO-d₆): δ ppm 9.12 (s, 1H), 8.03 (s, 1H), 7.52-7.20 (m, 2H), 6.95-6.79 (m, 2H), 6.77-6.66 (m, 2H), 2.96-2.84 (m, 1H), 1.78 (s, 3H), 1.71 (s, 3H), 0.84-0.74 (m, 2H), 0.56-0.44 (m, 2H); MS (ESI) m/z: 322.25 [M+H]⁺;]⁺; Chiral purity 99.8% (CHIRAL ART Cellulose-SB, 4.6×50 mm column, hexane with 0.3% IPA-amine/15% MeOH/DCM (1:1)). Compounds (72-B) and (72-C) are atropisomers, and the stereochemistry may be the opposite of that depicted.

### Example 73

### (R)-3-(4-Amino-6-(dimethylamino)pyrido[3,2-d]pyrimidin-8-yl)-2,4-dimethylphenol (73-B) and (S)-3-(4-Amino-6-(dimethylamino)pyrido[3,2-d]pyrimidin-8-yl)-2,4-dimethylphenol (73-C)

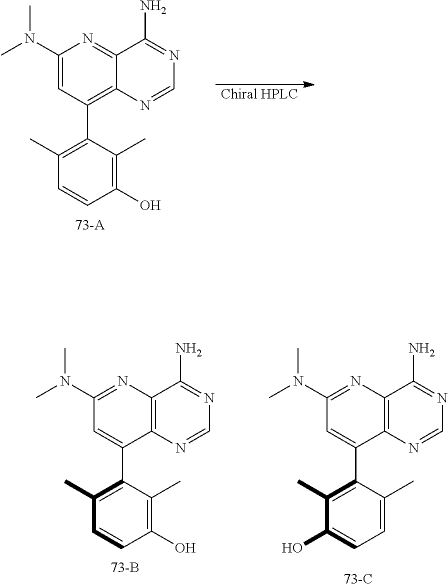

### Isolation of (R)-3-(4-amino-6-(dimethylamino)pyrido[3,2-d]pyrimidin-8-yl)-2,4-dimethylphenol (73-B) and (S)-3-(4-amino-6-(dimethylamino)pyrido[3,2-d]pyrimidin-8-yl)-2,4-dimethylphenol (73-C)

The racemic mixture of 3-(4-amino-6-(dimethylamino)pyrido[3,2-d]pyrimidin-8-yl)-2,4-dimethylphenol (73-A) (80 mg) was prepared according to the procedure described in Example 72 and subjected to chiral SFC purification (CHIRALPAK AD-H, 30×250 mm column, CO₂/30% IPA with 0.1% isopropylamine) to afford (R)-3-(4-amino-6-methylpyrido[3,2-d]pyrimidin-8-yl)-2,4-dimethylphenol (73-B) (30.3 mg, 38%) and (S)-3-(4-amino-6-(dimethylamino)pyrido[3,2-d]pyrimidin-8-yl)-2,4-dimethylphenol (73-C) (22.9 mg, 27%). 73-B: ¹H NMR (400 MHz, DMSO-d₆): δ ppm 9.10 (s, 1H), 8.00 (s, 1H), 7.54-7.12 (m, 2H), 6.97 (s, 1H), 6.89 (d, J=8.4 Hz, 1H), 6.74 (d, J=8.0 Hz, 1H), 3.16 (s, 6H), 1.77 (s, 3H), 1.70 (s, 3H); MS (ESI) m/z: 310.31 [M+H]⁺; Chiral purity 98.4% (CHIRALPAK AD-H, 4.6×250 mm column, CO₂/30% IPA with 0.2% isopropylamine). 73-C: ¹H NMR (400 MHz, DMSO-d₆): δ ppm 9.08 (s, 1H), 8.00 (s, 1H), 7.52-7.12 (m, 2H), 6.96 (s, 1H), 7.88 (d, J=8.4 Hz, 1H), 6.74 (d, J=8.0 Hz, 1H), 3.16 (s, 6H), 1.77 (s, 3H), 1.70 (s, 3H); MS (ESI) m/z: 310.31 [M+H]⁺; Chiral purity 96.3% (CHIRALPAK AD-H, 4.6×250 mm column, CO₂/30% IPA with 0.2% isopropylamine). Compounds (73-B) and (73-C) are atropisomers, and the stereochemistry may be the opposite of that depicted.

### Example 74

### (R)-3-(4-amino-6-methylpyrido[3,2-d]pyrimidin-8-yl)-2,4-dimethylphenol (74-B) and (S)-3-(4-amino-6-methylpyrido[3,2-d]pyrimidin-8-yl)-2,4-dimethylphenol (74-C)

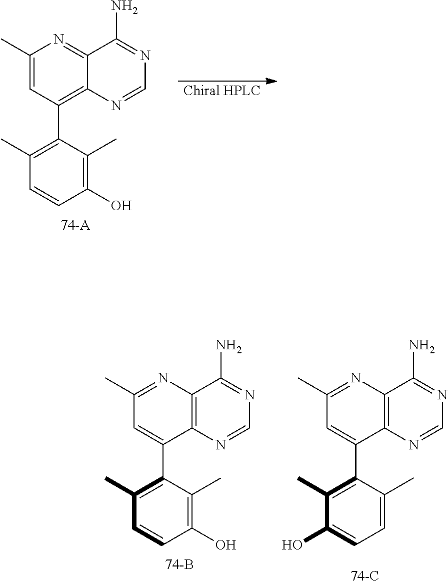

### Isolation of (R)-3-(4-amino-6-methylpyrido[3,2-d]pyrimidin-8-yl)-2,4-dimethylphenol (74-B) and (S)-3-(4-amino-6-methylpyrido[3,2-d]pyrimidin-8-yl)-2,4-dimethylphenol (74-C)

The racemic mixture of 3-(4-amino-6-methylpyrido[3,2-d]pyrimidin-8-yl)-2,4-dimethylphenol (74-A) (75 mg) was prepared according to the procedure described in Example 71 and was subjected to chiral SFC purification [Lux Cellulose-4, 30×250 mm column, CO₂/305% MeOH with 0.1% methanolic ammonia (7N)] to afford (R)-3-(4-amino-6-methylpyrido[3,2-d]pyrimidin-8-yl)-2,4-dimethylphenol (74-B) (23.3 mg, 31%) and (S)-3-(4-amino-6-methylpyrido[3,2-d]pyrimidin-8-yl)-2,4-dimethylphenol (74-C) (23.5 mg, 31%). 74-B: ¹H NMR (400 MHz, DMSO-d₆): δ ppm 9.15 (s, 1H), 8.23 (s, 1H), 7.80 (br s, 1H), 7.67 (br s, 1H), 7.44 (s, 1H), 6.90 (d, J=8.4 Hz, 1H), 6.76 (d, J=8.0 Hz, 1H), 2.67 (s, 3H), 1.73 (s, 3H), 1.65 (s, 3H); MS (ESI) m/z: 281.28 [M+H]⁺; Chiral purity 99.9% (Lux Cellulose-4, 4.6×150 mm column, CO2/35% MeOH with 0.5% DEA). 74-C: ¹H NMR (400 MHz, DMSO-d₆): δ ppm 9.15 (s, 1H), 8.23 (s, 1H), 7.80 (br s, 1H), 7.67 (br s, 1H), 7.44 (s, 1H), 6.90 (d, J=8.4 Hz, 1H), 6.76 (d, J=8.0 Hz, 1H), 2.67 (s, 3H), 1.72 (s, 3H), 1.65 (s, 3H); MS (ESI) m/z: 281.28 [M+H]⁺; Chiral purity 99.8%. Compounds (74-B) and (74-C) are atropisomers, and the stereochemistry may be the opposite of that depicted.

### Example 75

### (R)-3-(4-amino-6-(cyclopropyl(methyl)amino)pyrido[3,2-d]pyrimidin-8-yl)-2,4-dimethylphenol (75-B) and (S)-3-(4-amino-6-(cyclopropyl(methyl)amino)pyrido[3,2-d]pyrimidin-8-yl)-2,4-dimethylphenol

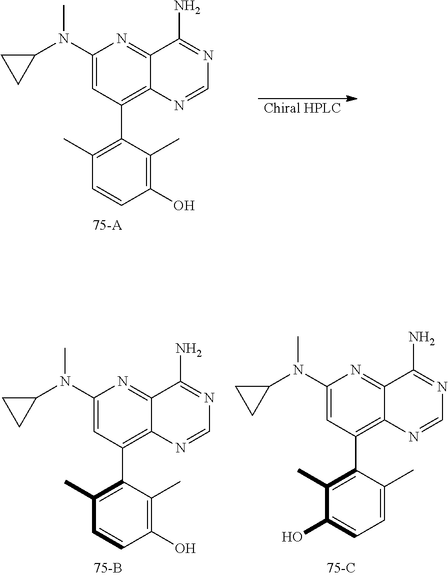

### Isolation of (R)-3-(4-amino-6-(cyclopropyl(methyl)amino)pyrido[3,2-d]pyrimidin-8-yl)-2,4-dimethylphenol (75-B) and (S)-3-(4-amino-6-(cyclopropyl(methyl)amino)pyrido[3,2-d]pyrimidin-8-yl)-2,4-dimethylphenol (75-C)

The racemic mixture of 3-(4-amino-6-(cyclopropyl(methyl)amino)pyrido[3,2-d]pyrimidin-8-yl)-2,4-dimethylphenol (75-A) (0.11 g, 58%) was prepared following the procedure described in Example 72 and was subjected to chiral SFC purification [CHIRALCEL-OX—H, 30×250 mm column, CO₂/40% MeOH with 0.1% methanolic ammonia (7N)] to afford (R)-3-(4-amino-6-(cyclopropyl(methyl)amino)pyrido[3,2-d]pyrimidin-8-yl)-2,4-dimethylphenol (75-B) (28 mg, 25%) and (S)-3-(4-amino-6-(cyclopropyl(methyl)amino)pyrido[3,2-d]pyrimidin-8-yl)-2,4-dimethylphenol (75-C) (33 mg, 30%). 75-B: ¹H NMR (400 MHz, DMSO-d₆): δ ppm 9.12 (s, 1H), 8.03 (s, 1H), 7.42 (br s, 1H), 7.23 (s, 2H), 6.90 (d, J=8.0 Hz, 1H), 6.75 (d, J=8.0 Hz, 1H), 3.22 (s, 3H), 2.72-2.64 (m, 1H), 1.79 (s, 3H), 1.71 (s, 3H), 0.91-0.87 (m, 2H), 0.67-0.62 (m, 2H); MS (ESI) m/z: 336.34 [M+H]⁺; Chiral purity 99.5% (CHIRALCEL-OX—H, 4.6×250 mm column, CO₂/40% MeOH with 0.5% DEA). 75-C: ¹H NMR (400 MHz, DMSO-d₆): δ ppm 9.13 (s, 1H), 8.03 (s, 1H), 7.44 (br s, 1H), 7.23 (s, 2H), 6.90 (d, J=8.0 Hz, 1H), 6.75 (d, J=8.0 Hz, 1H), 3.22 (s, 3H), 2.72-2.64 (m, 1H), 1.79 (s, 3H), 1.71 (s, 3H), 0.91-0.87 (m, 2H), 0.67-0.62 (m, 2H); MS (ESI) m/z: 336.34 [M+H]⁺; Chiral purity 97.03% (CHIRALCEL-OX—H, 4.6×250 mm column, CO₂/40% MeOH with 0.5% DEA). Compounds (75-B) and (75-C) are atropisomers, and the stereochemistry may be the opposite of that depicted.

### Example 76

### (R)-3-(4-amino-6-cyclopropylpyrido[3,2-d]pyrimidin-8-yl)-2,4-dimethylphenol (76-B) and (S)-3-(4-amino-6-cyclopropylpyrido[3,2-d]pyrimidin-8-yl)-2,4-dimethylphenol (76-C)

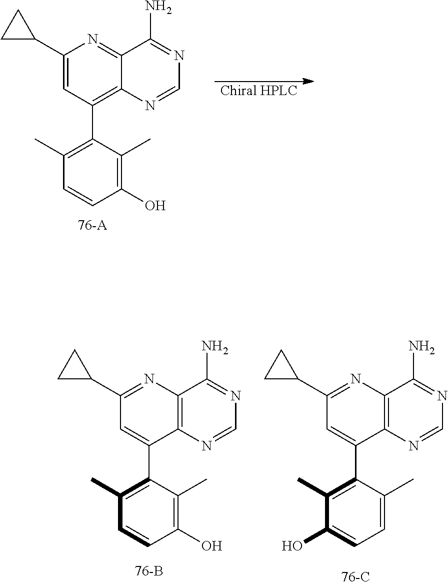

### Isolation of (R)-3-(4-amino-6-cyclopropylpyrido[3,2-d]pyrimidin-8-yl)-2,4-dimethylphenol (76-B) and (S)-3-(4-amino-6-cyclopropylpyrido[3,2-d]pyrimidin-8-yl)-2,4-dimethylphenol (76-B)

The racemic mixture of 3-(4-amino-6-cyclopropylpyrido[3,2-d]pyrimidin-8-yl)-2,4-dimethylphenol (76-A) was prepared according to the procedure described in Example 71 and was subjected to chiral SFC purification [CHIRALPAK IH, 30×250 mm column, CO₂/30% MeOH with 1% methanolic ammonia (2M)] to afford (R)-3-(4-amino-6-cyclopropylpyrido[3,2-d]pyrimidin-8-yl)-2,4-dimethylphenol (76-B) (37.3 mg, 38%) and (S)-3-(4-amino-6-cyclopropylpyrido[3,2-d]pyrimidin-8-yl)-2,4-dimethylphenol (76-C) (40.5 mg, 41%). 76-B: IH NMR (400 MHz, DMSO-d₆): δ ppm 9.15 (s, 1H), 8.20 (s, 1H), 7.92-7.55 (m, 2H), 7.49 (s, 1H), 6.91 (d, J=8.4 Hz, 1H), 6.77 (d, J=8.4 Hz, 1H), 2.35-2.26 (m, 1H), 1.75 (s, 3H), 1.67 (s, 3H), 1.30-1.17 (m, 2H), 1.10-1.02 (m, 2H); MS (ESI) m/z: 307.25 [M+H]⁺; Chiral purity 100% (CHIRALPAK AS-3, 3.0×50 mm column, CO₂/10-50% MeOH with 0.1% DEA). 76-C: IH NMR (400 MHz, DMSO-d₆): δ ppm 9.14 (s, 1H), 8.20 (s, 1H), 7.95-7.55 (m, 2H), 7.49 (s, 1H), 6.91 (d, J=8.0 Hz, 1H), 6.77 (d, J=8.0 Hz, 1H), 2.36-2.23 (m, 1H), 1.74 (s, 3H), 1.67 (s, 3H), 1.25-1.18 (m, 2H), 1.10-1.02 (m, 2H); MS (ESI) m/z: 307.30 [M+H]⁺; Chiral purity 98.8% (CHIRALPAK AS-3, 3.0×50 mm column, CO₂/10-50% MeOH with 0.1% DEA). Compounds (76-B) and 76-C) are atropisomers, and the stereochemistry may be the opposite of that depicted.

### Example 77

### Kinase Binding Assay

Test compound IC₅₀ values for PKMYT1 and WEE1 were determined by LanthaScreen® Eu Kinase Binding Assay (Thermo Fisher Scientific), based on the binding and displacement of a proprietary, AlexaFluor 647-labeled ATP-competitive kinase inhibitor scaffold (kinase tracer) to the kinase of interest. Binding of the tracer to the kinase is detected using a europium-labeled anti-GST antibody, which binds to the fused tag on the kinase protein. A high degree of FRET (Forster Resonance Energy Transfer) from the europium (Eu) donor fluorophore to the Alexa Fluor® 647 acceptor fluorophore on the kinase tracer results from concurrent binding of both the tracer and antibody to the kinase. Inhibitor binding to the kinase competes with tracer binding, resulting in loss of FRET from the donor/acceptor pairs.

Test compounds and positive control RP-6306 (Lunresertib) were screened in 1% DMSO (final concentration) in each well typically in 10-point titrations by 3-fold serial dilutions conducted from starting/top concentration of 10 mM. LanthaScreen® assay conditions for both PKMYT1 and WEE1 were conducted similarly in buffer comprised of 50 mM HEPES pH 7.5, 0.01% BRIJ-35, 10 mM MgCl₂, and 1 mM EGTA using AlexaFluor Tracer 178. Kinase/Antibody Mixtures and Tracer are diluted to a 2× and 4× working concentration, respectively in this same buffer. The final concentrations for the kinase, antibody, and tracer for PKMYT1 LanthaScreen® assay were 2.5, 2, and 1 nM, respectively and for the WEE1 LanthaScreen® assay these values were 5, 2, and 50 nM, respectively. Test compounds, kinase buffer, kinase/antibody mixtures, and tracer were added to individual wells of a bar-coded, low volume, white 384-well plates (Greiner Cat. #784297 or Corning Cat. #4513) to a final volume of 16 uL, shaken for 30 seconds, and incubated for 60 minutes at rt prior to reading on a suitable fluorescent plate reader and subsequent data analysis. 0% displacement (no known inhibitor) and 100% displacement (highest concentration of known inhibitor) controls were included on each plate for maximum and minimum Emission Ratio determination, respectively. Dasatinib was used for both PKMYT1 and WEE1 as the known inhibitor.

IC₅₀ determination was performed using a non-linear regression, variable slope (four parameter), inhibitor v. response equation (Prism 9.0). IC₅₀ values are provided in Table 2. In Table 2: A=0 nM<IC₅₀<10 nM; B=10 nM<IC₅₀≤100 nM; C═100 nM<IC₅₀≤1000 nM; D=1000 nM<IC₅₀; ND=Not Determined.

Based on the kinase activity data provided in Table 2, compounds of Formula (I), (Ia), (Ib), (Ic), (II), (II-1), (IIIa), (IIIb), (IIIc), (IIId), (IIIa-1), (IIIb-1), (IIIc-1), (IIId-1), (IVa), (IVa-1), (Va), (Va-1), (I′), (I′a), (I′b), (I′c), (II′), (II′-1), (III′a), (III′b), (III′c), (III′d), (III′a-1), (III′b-1), (III′c-1), (III′d-1), (IV′a), (IV′a-1), (V′a), (V′a-1), or Compound Nos. 1-A to 76-C provided herein are expected to exhibit excellent kinome selectivity which is a highly desirable property for a DNA damage response (DDR) kinase inhibitor to ensure a favorable in vivo safety profile.

TABLE 2
PKMYT1 and WEE1 IC₅₀ values.

| No. | PKMYT1 IC₅₀ (nM) | WEE1 IC₅₀ (nM) |
|---|---|---|
| RP-6306 | A | B |
| 1-B | B | D |
| 2-B | A | C |
| 3-B | A | D |
| 4-B | B | D |
| 5-B | B | D |
| 6-B | A | D |
| 7-B | A | D |
| 8-B | B | D |
| 9-B | B | D |
| 10-B | A | D |
| 11-B | A | D |
| 12-B | A | D |
| 13-E | B | D |
| 15-B | B | D |
| 16-B | B | D |
| 17-B | B | D |
| 18-B | A | D |
| 19-B | A | C |
| 20-B | A | C |
| 21-B | A | C |
| 22-B | B | D |
| 23-B | A | D |
| 24-B | B | D |
| 25-B | B | D |
| 26-B | B | D |
| 27-B | A | D |
| 28-B | A | D |
| 29-B | A | D |
| 30-B | A | D |
| 31-B | A | C |
| 32-B | A | C |
| 33-B | A | D |
| 34-B | C | D |
| 35-B | C | D |
| 36-B | A | C |
| 37-B | A | C |
| 38-B | A | D |
| 39-B | B | ND |
| 40-B | A | D |
| 41-B | A | D |
| 42-B | A | C |
| 43-B | A | D |
| 44-B | A | C |
| 45-B | B | D |
| 46-B | A | D |
| 47-A | B | D |
| 48-B | B | D |
| 49-B | B | D |
| 50-B | B | D |
| 51-B | B | D |
| 52-B | B | D |
| 53-B | B | D |
| 54-B | A | C |
| 55-B | A | C |
| 56-B | A | C |
| 57-B | A | D |
| 58-B | A | C |
| 59-B | A | C |
| 60-B | A | C |
| 61-B | A | D |
| 62-B | A | D |
| 62-D | A | D |
| 63-B | A | D |
| 71-B | A | C |
| 72-B | A | D |
| 73-B | B | D |
| 74-B | B | D |
| 75-B | B | D |
| 76-B | A | D |

All patents, published patent applications, and other non-patent documents referenced herein are each incorporated herein by reference in their entirety. Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. A compound represented by the following Formula (I): 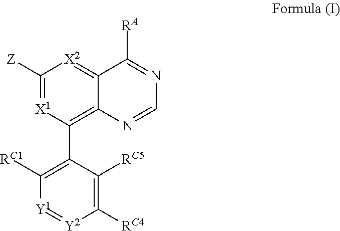
or a pharmaceutically acceptable salt thereof, wherein:
X¹ is —CR^{B1} or N;
X² is —CR^{B2} or N;
Y¹ is —CR^{C2} or N;
Y² is —CR^{C3} or N, provided that Y¹ and Y² are not both N;
Z is 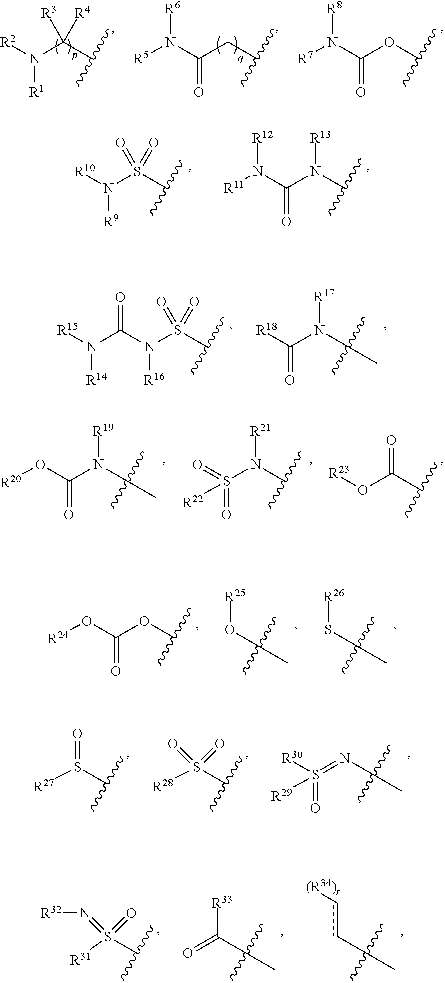
halogen, —CN, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl, wherein: [symbol]
is a double bond or a triple bond;
p and q are each independently 0 or 1;
r is 1 or 2;
R¹, R², R⁵, R⁶, R⁷, R⁸, R⁹, R¹⁰, R¹¹, R¹², R¹⁴, and R¹⁵ are each independently hydrogen, a substituted or an unsubstituted C₁-C₆ alkyl, a substituted or an unsubstituted C₁-C₆ heteroalkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 4- to 10-membered heterocyclyl, or a substituted or an unsubstituted 5- to 10-membered heteroaryl; or alternatively
R¹ and R², R⁵ and R⁶, R⁷ and R⁸, R⁹ and R¹⁰, R¹¹ and R¹², or R¹⁴ and R¹⁵, together with the respective nitrogen atom connected thereto, each independently form a substituted or an unsubstituted 4- to 10-membered heterocyclyl or a substituted or an unsubstituted 5- to 10-membered heteroaryl;
R³ and R⁴ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; or alternatively
R³ and R⁴, together with the carbon atom connected thereto, each independently form a substituted or an unsubstituted C₃-C₆ cycloalkyl or a substituted or an unsubstituted 4- to 6-membered heterocyclyl;
R¹⁵, R¹⁶, R⁷, R¹⁹, and R²¹ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, or a substituted or an unsubstituted C₃-C₆ cycloalkyl;
R¹⁸, R²⁰, R²³, R²⁴, R²⁵, and R²⁶ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl;
R²², R²⁷, R²⁸, R²⁹, R³⁰, and R³¹ are each independently a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl;
R³² is hydrogen, —CN, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl;
R³³ and R³⁴ are each independently hydrogen, a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₁-C₆ alkoxy, a substituted or an unsubstituted C₃-C₆ cycloalkyl, a substituted or an unsubstituted 6- or 10-membered aryl, a substituted or an unsubstituted 4- to 10-membered heterocyclyl, or a substituted or an unsubstituted 5- to 10-membered heteroaryl; and
R^{A} is —NR³⁵R³⁶, wherein:
R³⁵ and R³⁶ are each independently hydrogen, a substituted or an unsubstituted C₁-C₆ alkyl, a substituted or an unsubstituted C₃-C₆ cycloalkyl, or a substituted or an unsubstituted C₄-C₆ heterocyclyl; or alternatively
one of R³⁵ and R³⁶ is hydrogen or a substituted or an unsubstituted C₁-C₆ alkyl, and the other is a substituted or an unsubstituted 6- or 10-membered aryl or a substituted or an unsubstituted 5- to 10-membered heteroaryl;
R^{B1} and R^{B2} are each independently hydrogen, halogen, —CN, —OH, —OR³⁷, —SR³⁸, a substituted or an unsubstituted C₁-C₆ alkyl, a substituted or an unsubstituted C₁-C₆ heteroalkyl, a substituted or an unsubstituted C₂-C₆ alkenyl, a substituted or an unsubstituted C₂-C₆ alkynyl, a substituted or an unsubstituted C₃-C₆ cycloalkyl, or a substituted or an unsubstituted 5- to 10-membered heteroaryl, wherein:
R³⁷ and R³⁸, for each instance, are each independently a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, or a substituted or an unsubstituted C₄-C₆ heterocyclyl;
R^{C1}, R^{C2}, and R^{C3} are each independently hydrogen, halogen, —CN, —OH, —OR³⁹, —SR⁴⁰, a substituted or an unsubstituted C₁-C₆ alkyl, a substituted or an unsubstituted C₂-C₆ alkenyl, a substituted or an unsubstituted C₂-C₆ alkynyl, or a substituted or an unsubstituted C₃-C₆ cycloalkyl;
R^{C4} and R^{C5} are each independently hydrogen, halogen, —CN, —OH, —OR³⁹, —SR⁴⁰, a substituted or an unsubstituted C₁-C₆ alkyl, a substituted or an unsubstituted C₂-C₆ alkenyl, a substituted or an unsubstituted C₂-C₆ alkynyl, or a substituted or an unsubstituted C₁-C₆ alkoxy, a substituted or an unsubstituted C₃-C₆ cycloalkyl; or alternatively
R^{C4} and R^{C5}, together with the carbon atom that each of R^{C4} and R^{C5} is respectively connected thereto, form a substituted or an unsubstituted 5-membered heteroaryl, wherein:
R³⁹ and R⁴⁰, for each instance, are each independently a substituted or an unsubstituted C₁-C₆ alkyl, a substituted or an unsubstituted C₂-C₆ alkenyl, a substituted or an unsubstituted C₂-C₆ alkynyl, a substituted or an unsubstituted C₃-C₆ cycloalkyl, or a substituted or an unsubstituted C₄-C₆ heterocyclyl;
provided that R^{C1}, R^{C2}, R^{C3}, R^{C4} and R^{C5} are not all hydrogen; and
with the proviso that the compound represented by Formula (I) is not N-butyl-6-chloro-N-ethyl-8-(2,4,6-trimethylphenyl)-4-quinazolinamine.

2. The compound of claim 1, or a pharmaceutically acceptable salt thereof, wherein R³⁵ and R³⁶ are each independently hydrogen or a substituted or an unsubstituted C₁-C₆ alkyl.

3. The compound of claim 1 or 2, wherein the compound is represented by the following Formula (II): 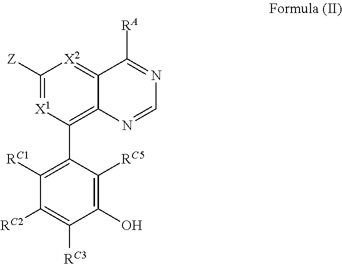
or a pharmaceutically acceptable salt thereof.

4. The compound of any one of claims 1 to 3, or a pharmaceutically acceptable salt thereof, wherein R^{C1}, R^{C2}, R^{C3}, and R^{C5} are each independently hydrogen, F, Cl, Br, —CN, or a substituted or an unsubstituted C₁-C₆ alkyl.

5. The compound of any one of claims 1 to 4, or a pharmaceutically acceptable salt thereof, wherein R^{C1}, R^{C2}, R^{C3}, and R^{C5} are each independently hydrogen, Cl, methyl, —CF₃, or —CHF₂.

6. The compound of any one of claims 1 to 5, wherein the compound is represented by the following Formula (IIIa) or Formula (IIIb): 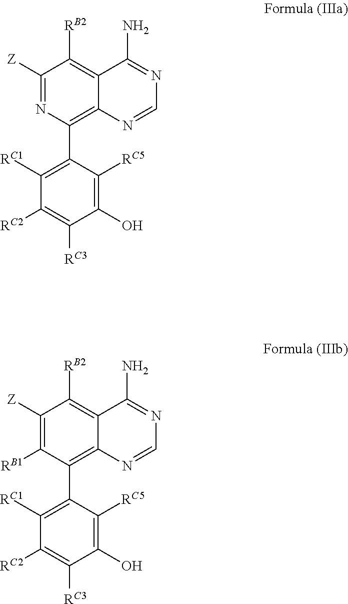
or a pharmaceutically acceptable salt thereof.

7. The compound of any one of claims 1 to 6, or a pharmaceutically acceptable salt thereof, wherein R^{B1} and R^{B2} are each independently hydrogen, F, Cl, Br, —CN, —OR³⁷, or a substituted or an unsubstituted C₁-C₆ alkyl, wherein:
R³⁷, for each instance, is independently a substituted or an unsubstituted C₁-C₆ alkyl.

8. The compound of any one of claims 1 to 7, or a pharmaceutically acceptable salt thereof, wherein R^{B1} is F, Cl, —CN, —OR³⁷, or a substituted or an unsubstituted C₁-C₄ alkyl, wherein:
R³⁷, for each instance, is independently a substituted or an unsubstituted C₁-C₄ alkyl.

9. The compound of any one of claims 1 to 8, or a pharmaceutically acceptable salt thereof, wherein R^{B2} is hydrogen, F, methyl, or methoxy.

10. The compound of any one of claims 1 to 9, or a pharmaceutically acceptable salt thereof, wherein Z is 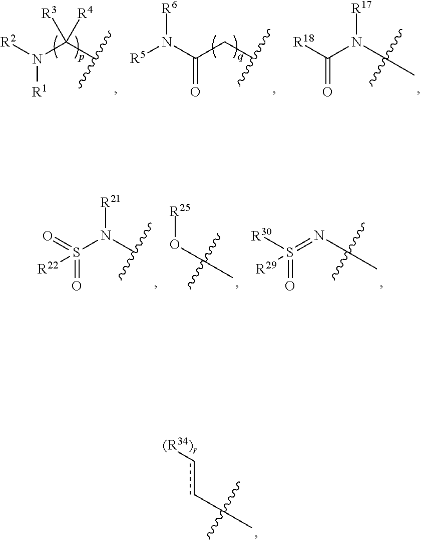
—CN, a substituted or an unsubstituted C₁-C₆ alkyl, a substituted or an unsubstituted C₂-C₆ alkenyl, a substituted or an unsubstituted C₃-C₆ cycloalkyl, a substituted or an unsubstituted phenyl, a substituted or an unsubstituted 4- to 10-membered heterocyclyl, or a substituted or an unsubstituted 5- to 10-membered heteroaryl.

11. The compound of any one of claims 1 to 10, or a pharmaceutically acceptable salt thereof, wherein Z is 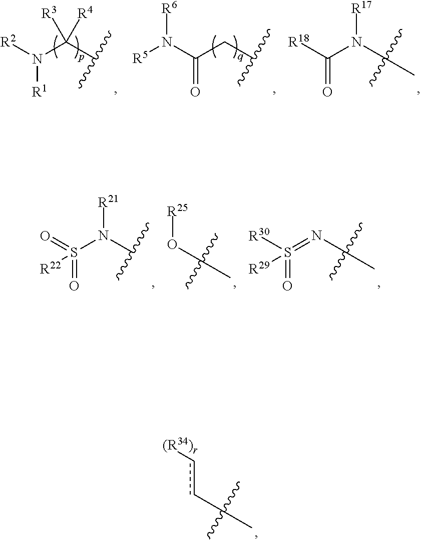
—CN, a substituted or an unsubstituted C₁-C₆ alkyl, a substituted or an unsubstituted C₃-C₆ cycloalkyl, a substituted or an unsubstituted phenyl, or a substituted or an unsubstituted 5- to 10-membered heteroaryl.

12. The compound of any one of claims 1 to 11, wherein the compound is represented by the following Formula (IVa): 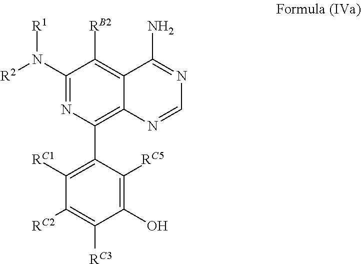
or a pharmaceutically acceptable salt thereof.

13. The compound of any one of claims 1 to 12, or a pharmaceutically acceptable salt thereof, wherein:
R¹ and R² are each independently hydrogen, a substituted or an unsubstituted C₁-C₆ alkyl, a substituted or an unsubstituted C₁-C₆ heteroalkyl, a substituted or an unsubstituted C₃-C₆ cycloalkyl, a substituted or an unsubstituted 4- to 10-membered heterocyclyl, or a substituted or an unsubstituted 5- to 10-membered heteroaryl, wherein:
when a C₁-C₆ alkyl or a C₁-C₆ heteroalkyl represented by R or R² is substituted, the C₁-C₆ alkyl or the C₁-C₆ heteroalkyl is substituted with one or more groups each independently selected from halogen, —CN, —OH, C₁-C₄ alkyl, C₁-C₄ haloalkyl, C₁-C₄ alkoxy, and C₃-C₆ cycloalkyl;
when a C₃-C₆ cycloalkyl, a 4- to 10-membered heterocyclyl, or a 5- to 10-membered heteroaryl represented by R¹ or R² is substituted, the C₃-C₆ cycloalkyl, the 4- to 10-membered heterocyclyl, or the 5- to 10-membered heteroaryl is substituted with one or more groups each independently selected from halogen, —CN, —OH, C₁-C₄ alkyl, C₁-C₄ alkyl substituted with one or more groups each independently selected from C₁-C₂ alkoxy, C₁-C₄ haloalkyl, and C₁-C₄ alkoxy; or alternatively
R¹ and R², together with the nitrogen atom connected thereto, form a substituted or an unsubstituted 4- to 8-membered heterocyclyl or a substituted or an unsubstituted 5- to 8-membered heteroaryl, wherein:
when a 4- to 8-membered heterocyclyl or a 5- to 8-membered heteroaryl represented by R¹ or R² is substituted, the 4- to 8-membered heterocyclyl or the 5- to 8-membered heteroaryl is substituted with one or more groups each independently selected from halogen, —CN, —OH, C₁-C₄ alkyl, C₁-C₄ alkyl substituted with one or more groups each independently selected from —OH, C₁-C₂ alkoxy, C₁-C₄ haloalkyl, and C₁-C₄ alkoxy.

14. The compound of any one of claims 1 to 13, or a pharmaceutically acceptable salt thereof, wherein:
R¹ and R² are each independently hydrogen, a substituted or an unsubstituted C₁-C₄ alkyl, a substituted or an unsubstituted C₃-C₅ cycloalkyl, a substituted or an unsubstituted 4- to 8-membered heterocyclyl, or a substituted or an unsubstituted 5- to 8-membered heteroaryl, wherein:
when a C₁-C₄ alkyl represented by R¹ or R² is substituted, the C₁-C₄ alkyl is substituted with one or more groups each independently selected from F, Cl, —CN, —OH, C₁-C₂ alkyl, C₁-C₂ haloalkyl, C₁-C₂ alkoxy, and C₃-C₄ cycloalkyl;
when a C₃-C₅ cycloalkyl, a 4- to 8-membered heterocyclyl, or a 5- to 8-membered heteroaryl represented by R¹ or R² is substituted, the C₃-C₅ cycloalkyl, the 4- to 8-membered heterocyclyl, or the 5- to 8-membered heteroaryl is substituted with one or more groups each independently selected from F, Cl, —CN, —OH, C₁-C₂ alkyl, C₁-C₂ alkyl substituted with one or more groups each independently selected from C₁-C₂ alkoxy, C₁-C₂ haloalkyl, and C₁-C₂ alkoxy; or alternatively
R¹ and R², together with the nitrogen atom connected thereto, form a substituted or an unsubstituted 4- to 8-membered heterocyclyl or a substituted or an unsubstituted 5- or 6-membered heteroaryl, wherein:
when a 4- to 8-membered heterocyclyl or a 5- or 6-membered heteroaryl formed by R¹ and R², together with the nitrogen atom connected thereto, is substituted, the 4- to 8-membered heterocyclyl or the 5- or 6-membered heteroaryl is substituted with one or more groups each independently selected from F, Cl, —CN, —OH, C₁-C₂ alkyl substituted with one or more groups each independently selected from —OH, C₁-C₂ alkoxy, C₁-C₂ haloalkyl, and C₁-C₂ alkoxy.

15. The compound of any one of claims 1 to 14, wherein the compound is represented by the following Formula (Va): 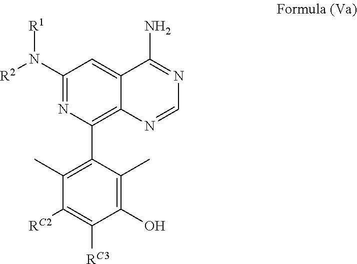
or a pharmaceutically acceptable salt thereof.

16. The compound of any one of claims 1 to 11, or a pharmaceutically acceptable salt thereof, wherein Z is

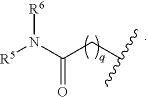

17. The compound of claim 16, or a pharmaceutically acceptable salt thereof, wherein:
R⁵ and R⁶ are each independently hydrogen or a substituted or an unsubstituted C₁-C₆ alkyl, wherein:
when a C₁-C₆ alkyl represented by R⁵ or R⁶ is substituted, the C₁-C₆ alkyl is substituted with one or more groups each independently selected from halogen, —CN, —OH, and C₁-C₄ alkoxy; or alternatively
R⁵ and R⁶, together with the nitrogen atom connected thereto, form a substituted or an unsubstituted 4- to 8-membered heterocyclyl, wherein:
when a 4- to 8-membered heterocyclyl represented by R¹ or R² is substituted, the 4- to 8-membered heterocyclyl is substituted with one or more groups each independently selected from halogen, —CN, —OH, C₁-C₄ alkyl substituted with one or more groups each independently selected from C₁-C₂ alkoxy, C₁-C₄ haloalkyl, and C₁-C₄ alkoxy.

18. The compound of any one of claims 1 to 11, or a pharmaceutically acceptable salt thereof, wherein Z is

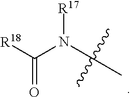

19. The compound of claim 18, or a pharmaceutically acceptable salt thereof, wherein:
R¹⁷ is hydrogen or a substituted or an unsubstituted C₁-C₆ alkyl; and
R¹⁸ is a substituted or an unsubstituted C₁-C₆ alkyl, wherein:
when a C₁-C₆ alkyl represented by R¹⁷ or R¹⁸ is substituted, the C₁-C₆ alkyl is substituted with one or more groups each independently selected from halogen, —CN, —OH, and C₁-C₄ alkoxy.

20. The compound of any one of claims 1 to 11, or a pharmaceutically acceptable salt thereof, wherein Z is

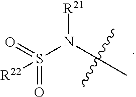

21. The compound of claim 20, or a pharmaceutically acceptable salt thereof, wherein:
R²¹ is hydrogen or a substituted or an unsubstituted C₁-C₆ alkyl; and
R²² is a substituted or an unsubstituted C₁-C₆ alkyl, wherein:
when a C₁-C₆ alkyl represented by R²¹ or R²² is substituted, the C₁-C₆ alkyl is substituted with one or more groups each independently selected from halogen, —CN, —OH, and C₁-C₄ alkoxy.

22. The compound of any one of claims 1 to 11, or a pharmaceutically acceptable salt thereof, wherein Z is

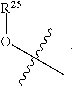

23. The compound of claim 22, or a pharmaceutically acceptable salt thereof, wherein:
R²⁵ is a substituted or an unsubstituted C₁-C₆ alkyl, wherein:
when a C₁-C₆ alkyl represented by R²⁵ is substituted, the C₁-C₆ alkyl is substituted with one or more groups each independently selected from halogen, —CN, —OH, and C₁-C₄ alkoxy.

24. The compound of any one of claims 1 to 11, or a pharmaceutically acceptable salt thereof, wherein Z is

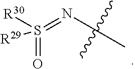

25. The compound of claim 24, or a pharmaceutically acceptable salt thereof, wherein:
R²⁹ and R³⁰ are each a substituted or an unsubstituted C₁-C₆ alkyl, wherein:
when a C₁-C₆ alkyl represented by R²⁹ or R³⁰ is substituted, the C₁-C₆ alkyl is substituted with one or more groups each independently selected from halogen, —CN, —OH, and C₁-C₄ alkoxy.

26. The compound of any one of claims 1 to 11, or a pharmaceutically acceptable salt thereof, wherein Z is

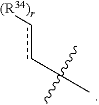

27. The compound of any one of claims 1 to 11, or a pharmaceutically acceptable salt thereof, wherein Z is

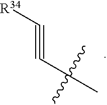

28. The compound of claim 26 or claim 27, or a pharmaceutically acceptable salt thereof, wherein:
R³⁴ is hydrogen, a substituted or an unsubstituted C₁-C₆ alkyl, or C₁-C₆ alkoxy, wherein:
when a C₁-C₆ alkyl represented by R³⁴ is substituted, the C₁-C₆ alkyl is substituted with one or more groups each independently selected from halogen, —CN, —OH, and C₁-C₄ alkoxy.

29. The compound of any one of claims 1 to 11, or a pharmaceutically acceptable salt thereof, wherein Z is —CN.

30. The compound of any one of claims 1 to 11, or a pharmaceutically acceptable salt thereof, wherein Z is a substituted or an unsubstituted C₁-C₆ alkyl, wherein:
when a C₁-C₆ alkyl represented by Z is substituted, the C₁-C₆ alkyl is substituted with one or more groups each independently selected from halogen, —CN, —OH, C₁-C₄ alkoxy, a C₃-C₆ cycloalkyl, and a 4- to 8-membered heterocyclyl.

31. The compound of any one of claims 1 to 11, or a pharmaceutically acceptable salt thereof, wherein Z is a substituted or an unsubstituted C₂-C₆ alkenyl, wherein:
when a C₂-C₆ alkenyl represented by Z is substituted, the C₂-C₆ alkenyl is substituted with one or more groups each independently selected from halogen, —CN, —OH, C₁-C₄ alkoxy, a C₃-C₆ cycloalkyl, and a 4- to 8-membered heterocyclyl.

32. The compound of any one of claims 1 to 11, or a pharmaceutically acceptable salt thereof, wherein Z is a substituted or an unsubstituted C₃-C₆ cycloalkyl; wherein:
when a C₃-C₆ cycloalkyl represented by Z is substituted, the C₃-C₆ cycloalkyl is substituted with one or more groups each independently selected from halogen, —CN, —OH, C₁-C₆ alkyl, C₁-C₆ alkyl substituted with one or more groups each independently selected from C₁-C₄ alkoxy, C₁-C₆ haloalkyl, and C₁-C₆ alkoxy.

33. The compound of any one of claims 1 to 11, or a pharmaceutically acceptable salt thereof, wherein Z is a substituted or an unsubstituted phenyl; wherein:
when a phenyl represented by Z is substituted, the phenyl is substituted with one or more groups each independently selected from halogen, —CN, —OH, C₁-C₄ alkyl, C₁-C₄ alkyl substituted with one or more groups each independently selected from C₁-C₂ alkoxy, C₁-C₄ haloalkyl, and C₁-C₄ alkoxy.

34. The compound of any one of claims 1 to 11, or a pharmaceutically acceptable salt thereof, wherein Z is a substituted or an unsubstituted 4- to 10-membered heterocyclyl; wherein:
when a 4- to 10-membered heterocyclyl represented by Z is substituted, the 4- to 10-membered heterocyclyl is substituted with one or more groups each independently selected from halogen, —CN, —OH, C₁-C₄ alkyl, C₁-C₄ alkyl substituted with one or more groups each independently selected from —OH, C₁-C₂ alkoxy, C₁-C₄ haloalkyl, and C₁-C₄ alkoxy.

35. The compound of any one of claims 1 to 11, or a pharmaceutically acceptable salt thereof, wherein Z is a substituted or an unsubstituted 5- to 10-membered heteroaryl; wherein:
when a 5- to 10-membered heteroaryl represented by Z is substituted, the 5- to 10-membered heteroaryl is substituted with one or more groups each independently selected from halogen, —CN, —OH, C₁-C₄ alkyl, C₁-C₄ alkyl substituted with one or more groups each independently selected from C₁-C₂ alkoxy, C₁-C₄ haloalkyl, and C₁-C₄ alkoxy.

36. The compound of claim 1, wherein the compound is selected from any one of Compound Nos. 1-A to 76-C in Table 1, or a pharmaceutically acceptable salt thereof.

37. A compound selected from any one of Compound Nos. 3A, 6A, 7A, 10A, 11A, 12A, 18A, 23A, 27A, 29A, 30A, 33A, 38A, 40A, 41A, 43A, 46A, 57A, 60A, 61A, 62A, 63A, 65A, 68A and 70A in Table 1, or a pharmaceutically acceptable salt thereof.

38. A compound selected from any one of Compound Nos. 3B, 6B, 7B, 10B, 11B, 12B, 18B, 23B, 27B, 29B, 30B, 33B, 38B, 40B, 41B, 43B, 46B, 57B, 60B, 61B, 62D, 63B, 65B, 68B and 70B in Table 1, or a pharmaceutically acceptable salt thereof.

39. The compound of any one of claims 1 to 38, or a pharmaceutically acceptable salt thereof, wherein one or more hydrogen atoms of the compound is substituted with deuterium.

40. A pharmaceutical composition comprising the compound of any one of claims 1 to 39, or a pharmaceutically acceptable salt thereof, and a pharmaceutically acceptable carrier, diluent, or a combination thereof.

41. A method for ameliorating or treating a cancer in a subject, comprising administering to the subject an effective amount of the compound of any one of claims 1 to 39, or a pharmaceutically acceptable salt thereof, or the pharmaceutical composition of claim 40.

42. A method for inhibiting replication of a malignant growth or tumor in a subject, comprising administering to the subject an effective amount of the compound of any one of claims 1 to 39, or a pharmaceutically acceptable salt thereof, or the pharmaceutical composition of claim 40; wherein the malignant growth or tumor is due to a cancer.

43. Use of the compound of any one of claims 1 to 39, or a pharmaceutically acceptable salt thereof, or the pharmaceutical composition of claim 40 for the manufacture of a medicament for ameliorating or treating a cancer.

44. Use of the compound of any one of claims 1 to 39, or a pharmaceutically acceptable salt thereof, or the pharmaceutical composition of claim 40 for the manufacture of a medicament for inhibiting replication of a malignant growth or tumor, wherein the malignant growth or tumor is due to a cancer.

45. The compound of any one of claims 1 to 39, or a pharmaceutically acceptable salt thereof, or the pharmaceutical composition of claim 40 for use in ameliorating or treating a cancer.

46. The compound of any one of claims 1 to 39, or a pharmaceutically acceptable salt thereof, or the pharmaceutical composition of claim 40 for use in inhibiting replication of a malignant growth or tumor, wherein the malignant growth or tumor is due to a cancer.

47. The method of claim 41 or 42, or the use of claim 43 or 44, or the compound of claim 45 or 46, wherein the cancer is selected from a brain cancer, a cervicocerebral cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.
